(19) United States
(12) Patent Application Publication
(10) Pub. No.: US 2022/0109109 A1
(43) Pub. Date: Apr. 7, 2022

(54) ORGANIC ELECTROLUMINESCENT COMPOUND, A PLURALITY OF HOST MATERIALS AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Chi-Sik KIM, Gyeonggi-do (KR); Kyung-Hoon CHOI, Gyeonggi-do (KR); Hyo-Nim SHIN, Gyeonggi-do (KR); Kyoung-Jin PARK, Gyeonggi-do (KR); Hyun-Woo KANG, Gyeonggi-do (KR); Soo-Yong LEE, Gyeonggi-do (KR); Seung-Hoon YOO, Gyeonggi-do (KR); Mi-Ja LEE, Gyeonggi-do (KR); Samuel KIM, Gyeonggi-do (KR)

(21) Appl. No.: 17/405,987

(22) Filed: Aug. 18, 2021

(30) Foreign Application Priority Data
Aug. 21, 2020 (KR) 10-2020-0105131
Jun. 29, 2021 (KR) 10-2021-0084629

(51) Int. Cl. H01L 51/00

(52) U.S. Cl. CPC H01L 51/0067; H01L 51/0072; H01L 51/5012; H01L 51/0074; H01L 2251/5384; H01L 51/0073

(57) ABSTRACT

The present disclosure relates to an organic electroluminescent compound represented by formula 1′ or 2′, a plurality of host materials comprising at least one first host compound and at least one second host compound, and an organic electroluminescent device comprising the same. By comprising the organic electroluminescent compound or the specific combination of compounds according to the present disclosure as a host material(s), it is possible to provide an organic electroluminescent device having improved driving voltage, luminous efficiency, power efficiency and/or lifetime properties.

### TECHNICAL FIELD

The present disclosure relates to an organic electroluminescent compound, a plurality of host materials, and an organic electroluminescent device comprising the same.

### BACKGROUND ART

A small molecular green organic electroluminescent device (OLED) was first developed by Tang, et al., of Eastman Kodak in 1987 by using TPD/Alq3 bi-layer consisting of a light-emitting layer and a charge transport layer. Thereafter, the development of OLEDs was rapidly effected and OLEDs have been commercialized. At present, OLEDs primarily use phosphorescent materials having excellent luminous efficiency in panel implementation. An OLED having high luminous efficiency and/or long lifetime is required for long time use and high resolution of a display.

In order to improve luminous efficiency, driving voltage and/or lifetime, various materials or concepts for an organic layer of an organic electroluminescent device have been proposed, but they have not been satisfactory in practical use. Thus, there is a continuous need to develop an organic electroluminescent device having improved performance, for example, improved driving voltage, luminous efficiency, power efficiency, and/or lifetime properties, compared to the conventional organic electroluminescent device.

Meanwhile, Chinese Patent No. 103467450 and Korean Patent Application Laid-Open No. 2011-0122051 disclose a compound in which a nitrogen-containing heteroaryl is bonded to a biscarbazole moiety, but fail to specifically disclose the specific combination of host materials claimed herein.

### DISCLOSURE OF INVENTION

### Technical Problem

The objective of the present disclosure is to provide an organic electroluminescent compound having a new structure suitable for applying it to an organic electroluminescent device. Another objective of the present disclosure is to provide an organic electroluminescent device having lower driving voltage, higher luminous efficiency, higher power efficiency and/or improved lifetime properties by comprising a specific combination of compounds according to the present disclosure as a plurality of host materials.

### Solution to Problem

As a result of intensive studies to solve the technical problems, the present inventors found that the above objective can be achieved by an organic electroluminescent compound represented by the following formula 1′ or 2′. In addition, the present inventors found that the above objective can be achieved by a plurality of host materials comprising at least one first host compound and at least one second host compound, wherein the first host compound is represented by the following formula 1, and the second host compound is represented by the following formula 2, at least one of formula 1 and formula 2 contains deuterium, and the first host compound and the second host compound are different from each other.

[A]Dⁿ¹-[B]Dⁿ² (1)

In formula 1,

A represents *-L₁-HAr;

L₁ represents a single bond, a substituted or unsubstituted (C1-C30)alkylene, a substituted or unsubstituted (C6-C30)arylene, or a substituted or unsubstituted (3- to 30-membered)heteroarylene;

HAr represents a substituted or unsubstituted nitrogen-containing (3- to 30-membered)heteroaryl;

B is represented by the following formula 1-a:

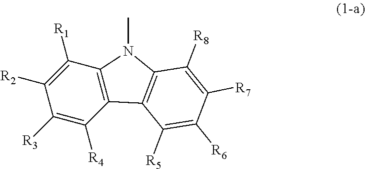

in formula 1-a,

R₁ to R₈, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl, with the proviso that at least one of R₁ to R₈ is represented by the following formula 1-b:

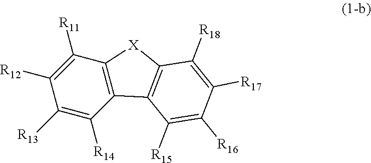

in formula 1-b,

X represents O, S, CR₂₁R₂₂, SiR₂₃R₂₄, or NR₂₅;

R₁₁ to R₁₈, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl, with the proviso that at least one of R₁₁ to R₁₈ is linked to formula 1-a;

R₂₁ to R₂₅, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl, or may be linked to an adjacent substituent(s) to form a ring(s);

[A]Dⁿ¹ and [B]Dⁿ² represent that A is substituted with n1 deuterium, and B is substituted with n2 deuterium, respectively; n1 and n2, each independently, represent an integer of 0 to 50, with the proviso that when formula 1 contains deuterium, at least one of n1 and n2 is an integer of 5 or more; and

A and B are linked to each other at the position of *.

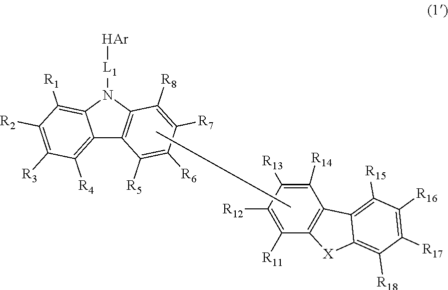

In formula 1′,

R₁ to R₈, and R₁₁ to R₁₈, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl,

any one of R₅ to R₈ is linked to any one of R₁₁ to R₁₄ to form a single bond,

with the proviso that at least five of R₁ to R₈ and R₁₁ to R₁₈ are deuterium; and

L₁, HAr, and X are as defined in formula 1 above.

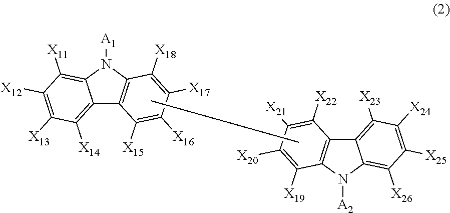

In formula 2,

A₁ and A₂, each independently, represent a substituted or unsubstituted (C6-C30)aryl, a substituted or unsubstituted dibenzofuranyl, a substituted or unsubstituted dibenzothiophenyl, or a substituted or unsubstituted carbazolyl;

X₁₁ to X₂₆, each independently, represent hydrogen, deuterium, a substituted or unsubstituted (C6-C30)aryl, or a substituted or unsubstituted (3- to 30-membered)heteroaryl, or may be linked to an adjacent substituent(s) to form a ring(s); and

any one of X₁₁ to X₁₈ is linked to any one of X₁₉ to X₂₆ to form a single bond;

with the proviso that when formula 2 contains deuterium, at least four of X₁₁ to X₂₆ are deuterium, and at least one of X₁₁, X₁₈, X₁₉, and X₂₆ is deuterium.

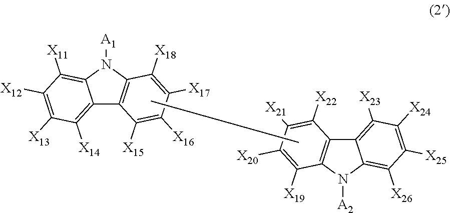

In formula 2′,

A₁ and A₂, each independently, represent a substituted or unsubstituted (C6-C30)aryl, a substituted or unsubstituted dibenzofuranyl, a substituted or unsubstituted dibenzothiophenyl, or a substituted or unsubstituted carbazolyl;

X₁₁ to X₂₆, each independently, represent hydrogen, deuterium, a substituted or unsubstituted (C6-C30)aryl, or a substituted or unsubstituted (3- to 30-membered)heteroaryl; and

any one of X₁₁ to X₁₈ is linked to any one of X₁₉ to X₂₆ to form a single bond;

with the proviso that at least four of X₁₁ to X₂₆ are deuterium, and at least one of X₁₁, X₁₈, X₁₉, and X₂₆ is deuterium.

### Advantageous Effects of Invention

The organic electroluminescent compound according to the present disclosure exhibits the performances suitable for using it in an organic electroluminescent device. In addition, by comprising a plurality of host materials according to the present disclosure, an organic electroluminescent device having lower driving voltage, higher luminous efficiency, higher power efficiency, and/or improved lifetime properties compared to conventional organic electroluminescent devices can be provided, and it is possible to produce a display system or a lighting system using the same.

### BRIEF DESCRIPTION OF DRAWINGS

FIG. 1 illustrates a graph showing the increase in bond dissociation energy according to deuteration.

### MODE FOR THE INVENTION

Hereinafter, the present disclosure will be described in detail. However, the following description is intended to explain the invention and is not meant in any way to restrict the scope of the present disclosure.

The term “organic electroluminescent compound” in the present disclosure means a compound that may be used in an organic electroluminescent device, and may be comprised in any layer constituting an organic electroluminescent device, as necessary.

The term “an organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of organic electroluminescent materials” in the present disclosure means an organic electroluminescent material comprising a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials of the present disclosure may be a combination of at least two compounds, which may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. The at least two compounds may be comprised in the same layer or different layers, and may be mixture-evaporated or co-evaporated, or may be individually evaporated.

The term “a plurality of host materials” in the present disclosure means an organic electroluminescent material comprising a combination of at least two host materials. It may mean both a material before being comprised in an organic electroluminescent device (for example, before vapor deposition) and a material after being comprised in an organic electroluminescent device (for example, after vapor deposition). The plurality of host materials of the present disclosure may be comprised in any light-emitting layer constituting an organic electroluminescent device. At least two compounds comprised in the plurality of host materials of the present disclosure may be comprised together in one light-emitting layer or may respectively be comprised in different light-emitting layers. When at least two host materials are comprised in one layer, for example, they may be mixture-evaporated to form a layer or may be separately co-evaporated simultaneously to form a layer.

Herein, the term “(C1-C30)alkyl(ene)” is meant to be a linear or branched alkyl(ene) having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. The term “(C2-C30)alkenyl” is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cycloheptylmethyl, cyclohexylmethyl, etc. The term “(3- to 7-membered)heterocycloalkyl” is meant to be a cycloalkyl having 3 to 7, preferably 5 to 7, ring backbone atoms, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc. The term “(C6-C30)aryl(ene)” is meant to be a monocyclic or fused ring radical derived from an aromatic hydrocarbon having 6 to 30 ring backbone carbon atoms, in which the number of the ring backbone carbon atoms is preferably 6 to 25, and more preferably 6 to 18. The above aryl(ene) may be partially saturated, and may comprise a spiro structure. The above aryl may include phenyl, biphenyl, terphenyl, naphthyl, binaphthyl, phenylnaphthyl, naphthylphenyl, phenylterphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, azulenyl, tetramethyldihydrophenanthrenyl, etc. More specifically, the above aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a] fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl(ene)” is meant to be an aryl(ene) having 3 to 30 ring backbone atoms, and including at least one, preferably 1 to 4 heteroatoms selected from the group consisting of B, N, O, S, Si, and P. The above heteroaryl(ene) may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, and pyridazinyl, and a fused ring-type heteroaryl such as benzofuranyl, benzothiophenyl, isobenzofuranyl, dibenzofuranyl, dibenzothiophenyl, dibenzoselenophenyl, naphthobenzofuranyl, naphthobenzothiophenyl, benzofuroquinolyl, benzofuroquinazolinyl, benzofuronaphthyridinyl, benzofuropyrimidinyl, naphthofuropyrimidinyl, benzothienoquinolyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzimidazolyl, benzothiazolyl, benzoisothiazolyl, benzoisoxazolyl, benzoxazolyl, isoindolyl, indolyl, benzoindolyl, indazolyl, benzothiadiazolyl, quinolyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, dihydroacridinyl, benzotriazolephenazinyl, imidazopyridyl, chromenoquinazolinyl, thiochromenoquinazolinyl, dimethylbenzoperimidinyl, indolocarbazolyl, indenocarbazolyl, etc. More specifically, the above heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolydinyl, 2-indolydinyl, 3-indolydinyl, 5-indolydinyl, 6-indolydinyl, 7-indolydinyl, 8-indolydinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Furthermore, “halogen” includes F, Cl, Br, and I.

In addition, “ortho (o-),” “meta (m-),” and “para (p-)” are prefixes, which represent the relative positions of substituents respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

Herein, “substituted” in the expression “substituted or unsubstituted” means that a hydrogen atom in a certain functional group is replaced with another atom or another functional group, i.e., a substituent, and also includes that the hydrogen atom is replaced with a group formed by a linkage of two or more substituents of the above substituents. For example, the “group formed by a linkage of two or more substituents” may be pyridine-triazine. That is, pyridine-triazine may be interpreted as one heteroaryl substituent, or as substituents in which two heteroaryl substituents are linked. Herein, the substituent(s) of the substituted alkyl(ene), the substituted aryl(ene), the substituted heteroaryl(ene), the substituted nitrogen-containing heteroaryl, the substituted cycloalkyl, the substituted cycloalkenyl, the substituted heterocycloalkyl, the substituted dibenzofuranyl, the substituted dibenzothiophenyl, and the substituted carbazolyl, each independently, are at least one selected from the group consisting of deuterium; a halogen; a cyano; a carboxyl; a nitro; a hydroxyl; a phosphineoxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of deuterium and a (C6-C30)aryl(s); a (C6-C30)aryl unsubstituted or substituted with at least one of deuterium and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldiyl(C6-C30)arylsilyl; a fused ring group of a (C3-C30) aliphatic ring(s) and a (C6-C30) aromatic ring(s); an amino; a mono- or di-(C1-C30)alkylamino; a mono- or di-(C2-C30)alkenylamino; a mono- or di-(C6-C30)arylamino unsubstituted or substituted with a (C1-C30)alkyl(s); a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituent(s), each independently, are at least one selected from the group consisting of deuterium; a (C1-C20)alkyl; a (5- to 25-membered)heteroaryl unsubstituted or substituted with a (C6-C25)aryl(s); a (C6-C25)aryl; and tri(C1-C30)arylsilyl. According to another embodiment of the present disclosure, the substituent(s), each independently, are at least one selected from the group consisting of deuterium; a (C1-C10)alkyl; a (5- to 20-membered)heteroaryl unsubstituted or substituted with a (C6-C18)aryl(s); a (C6-C25)aryl; and a tri(C1-C18)arylsilyl. Specifically, the substituent(s), each independently, may be at least one selected from the group consisting of deuterium; a methyl; a phenyl; a naphthyl; a biphenyl; a triphenylenyl; a pyridyl unsubstituted or substituted with a phenyl(s); a dibenzofuranyl; a dibenzothiophenyl; a carbazolyl unsubstituted or substituted with a phenyl(s); and a triphenylsilyl.

In the formulas of the present disclosure, when adjacent substituents are linked to each other to form a ring, the ring may be a substituted or unsubstituted mono- or polycyclic (3- to 30-membered) alicyclic or aromatic ring, or the combination thereof, to which two or more adjacent substituents are linked or fused. In addition, the formed ring may contain at least one heteroatom selected from B, N, O, S, Si, and P, and preferably at least one heteroatom selected from N, O, and S. According to one embodiment of the present disclosure, the number of ring backbone atoms is 5 to 20. According to another embodiment of the present disclosure, the number of ring backbone atoms is 5 to 15.

In the formulas of the present disclosure, heteroaryl, heteroarylene, and heterocycloalkyl, each independently, may contain at least one heteroatom selected from B, N, O, S, Si, and P. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldiyl(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-C30)arylamino.

The present disclosure provides an organic electroluminescent compound represented by formula 1′ or 2′. The organic electroluminescent compound of formula 2′ may be used in a light-emitting layer, a hole transport zone (comprising a hole transport layer, a hole auxiliary layer, and/or a light-emitting auxiliary layer), or an electron buffer layer, but is not limited thereto.

A plurality of host materials according to one embodiment of the present disclosure comprise a first host material including the compound represented by formula 1, and a second host material including a compound represented by formula 2, and the host materials may be comprised in a light-emitting layer of the organic electroluminescent device according to one embodiment of the present disclosure.

Hereinafter, the compound represented by formula 1 or 1′ will be described in more detail.

In formula 1, A represents *-L₁-HAr.

L₁ represents a single bond, a substituted or unsubstituted (C1-C30)alkylene, a substituted or unsubstituted (C6-C30)arylene, or a substituted or unsubstituted (3- to 30-membered)heteroarylene. According to one embodiment of the present disclosure, L₁ represents a single bond, a substituted or unsubstituted (C6-C18)arylene, or a substituted or unsubstituted (5- to 20-membered)heteroarylene. According to another embodiment of the present disclosure. L₁ represents a single bond, a (C6-C12)arylene unsubstituted or substituted with a (C6-C12)aryl(s), or an unsubstituted (5- to 15-membered)heteroarylene. For example, L₁ may represent a single bond, a phenylene unsubstituted or substituted with a phenyl(s), a naphthylene, a biphenylene, or a pyridylene. According to one embodiment of the present disclosure, L₁ may represent a single bond or may be represented by any one selected from the group consisting of the followings:

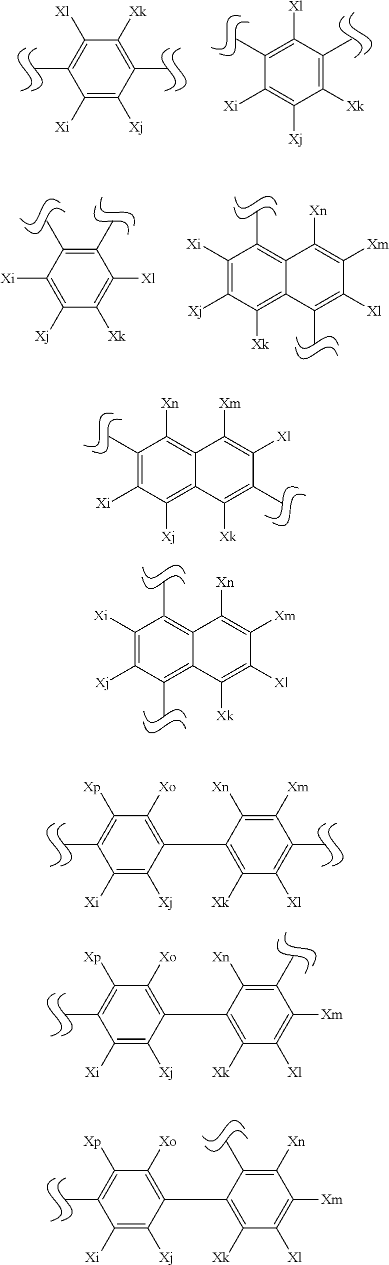

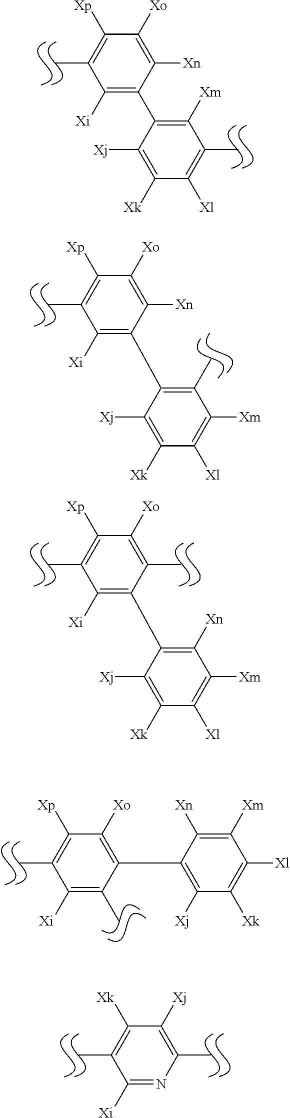

wherein, Xi to Xp, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C30)aryl, a substituted or unsubstituted (3- to 30-membered)heteroaryl, —NR₂₆R₂₇, or —SiR₂₈R₂₉R₃₀; or may be linked to an adjacent substituent(s) to form a ring(s); and R₂₆ to R₃₀, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl, or may be linked to an adjacent substituent(s) to form a ring(s).

HAr represents a substituted or unsubstituted nitrogen-containing (3- to 30-membered)heteroaryl. According to one embodiment of the present disclosure, HAr represents a substituted or unsubstituted nitrogen-containing (5- to 20-membered)heteroaryl. According to another embodiment of the present disclosure, HAr represents a nitrogen-containing (5- to 15-membered)heteroaryl unsubstituted or substituted with at least one selected from the group consisting of a (C6-C12)aryl and a (5- to 20-membered)heteroaryl unsubstituted or substituted with a (C6-C12)aryl(s). According to another embodiment of the present disclosure. HAr represents a substituted or unsubstituted triazinyl, a substituted or unsubstituted pyridyl, a substituted or unsubstituted pyrimidinyl, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted quinolyl, a substituted or unsubstituted benzoquinolyl, a substituted or unsubstituted isoquinolyl, a substituted or unsubstituted benzoisoquinolyl, a substituted or unsubstituted triazolyl, a substituted or unsubstituted pyrazolyl, a substituted or unsubstituted naphthyridinyl, a substituted or unsubstituted triazanaphthyl, a substituted or unsubstituted benzofuropyrimidinyl, or a substituted or unsubstituted benzothienopyrimidinyl. For example, HAr may be a substituted triazinyl, wherein the substituent may be at least one of a phenyl(s), a biphenyl(s), a dibenzofuranyl(s), a dibenzothiophenyl(s), and a phenylcarbazolyl(s), which may be further substituted with deuterium.

In formula 1, B is represented by the following formula 1-a.

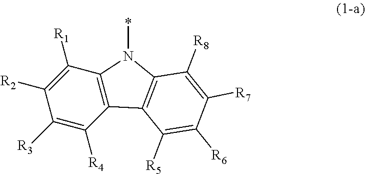

In formula 1-a. R₁ to R₈, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl, with the proviso that at least one of R₁ to R₈ is represented by the following formula 1-b. According to one embodiment of the present disclosure, R₁ to R₈, each independently, represent hydrogen, deuterium, or a substituted or unsubstituted (C6-C12)aryl, or are represented by the following formula 1-b. According to another embodiment of the present disclosure, R₁ to R₈, each independently, represent hydrogen, deuterium, or a (C6-C12)aryl unsubstituted or substituted with deuterium, or are represented by the following formula 1-b. For example, R₁ to R₈, each independently, may be hydrogen, deuterium, or a phenyl unsubstituted or substituted with deuterium, or may be represented by the following formula 1-b.

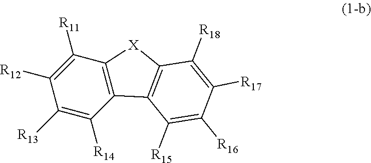

In formula 1-b, X represents O, S, CR₂₁R₂₂, SiR₂₃R₂₄, or NR₂₅. According to one embodiment of the present disclosure, X represents O or S.

In formula 1-b, R₂₁ to R₂₅, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl, or may be linked to an adjacent substituent(s) to form a ring(s).

In formula 1-b, R₁₁ to R₁₈, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl, with the proviso that at least one of R₁₁ to R₁₈ is linked to formula 1-a. According to one embodiment of the present disclosure, R₁₁ to R₁₆, each independently, represent hydrogen, deuterium, or a substituted or unsubstituted (C6-C12)aryl. According to another embodiment of the present disclosure, R₁₁ to R₁₈, each independently, represent hydrogen, deuterium, or a (C6-C12)aryl unsubstituted or substituted with deuterium. For example, R₁₁ to R₁₈, each independently, represent hydrogen, deuterium, or a phenyl unsubstituted or substituted with deuterium.

In formula 1, A and B are linked to each other at the position of *.

In formula 1, [A]Dⁿ¹ and [B]Dⁿ² represent that A is substituted with deuterium, in which the number of deuterium is n1, and B is substituted with deuterium, in which the number of deuterium is n2, respectively. According to one embodiment of the present disclosure, n1 and n2, each independently, represent an integer of 0 to 50. According to another embodiment of the present disclosure, the sum of n1 and n2 is an integer of 5 to 50. According to another embodiment of the present disclosure, at least one of n1 and n2 is an integer of 5 or more.

According to one embodiment of the present disclosure, B may be represented by at least one of the following formulas B-1 to B-16.

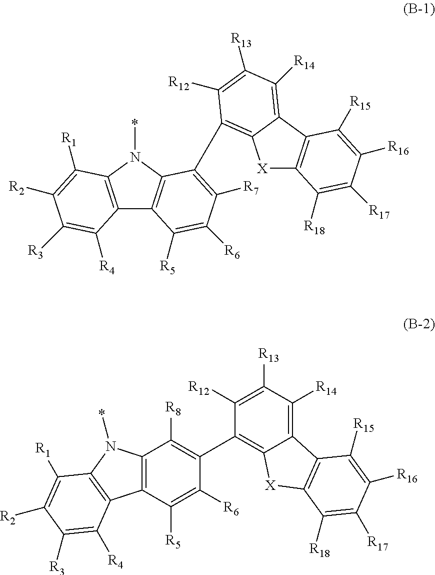

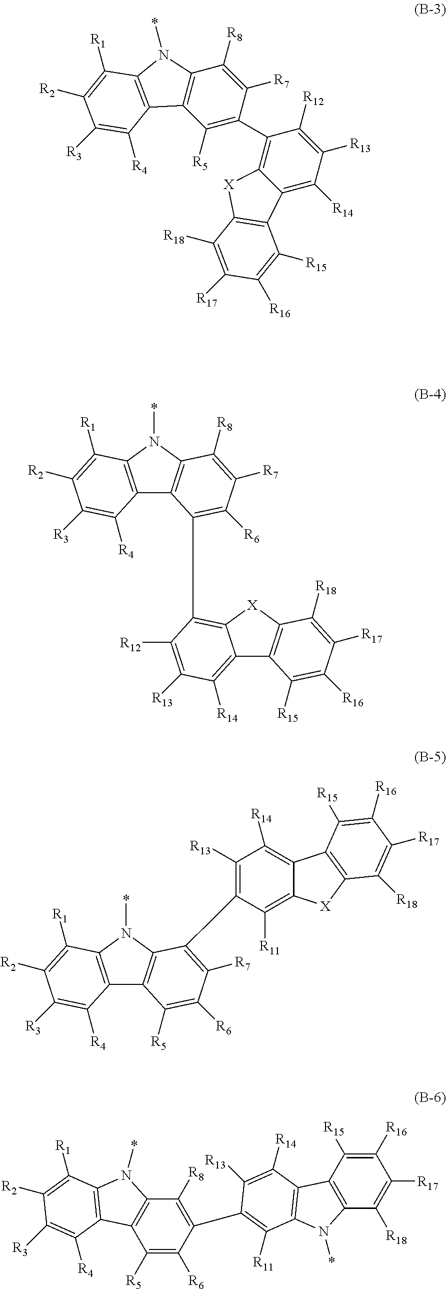

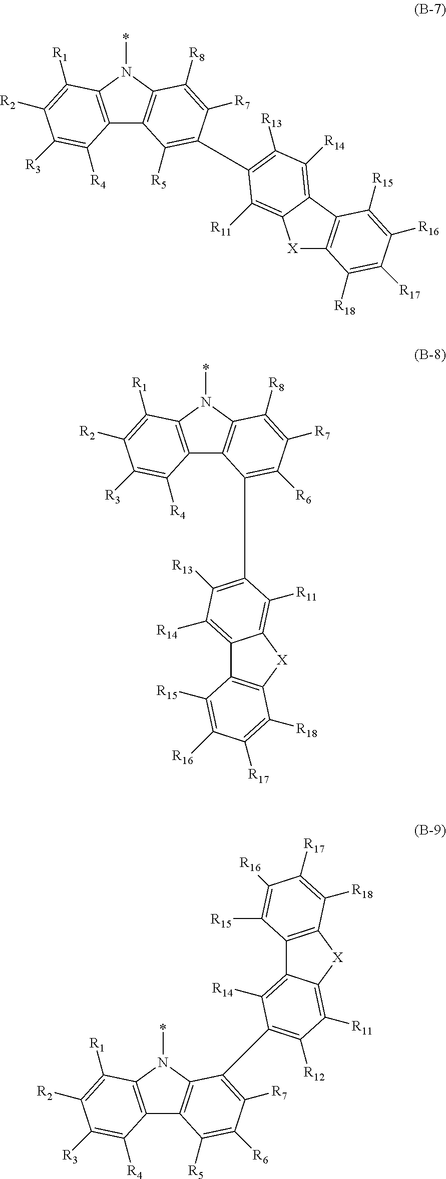

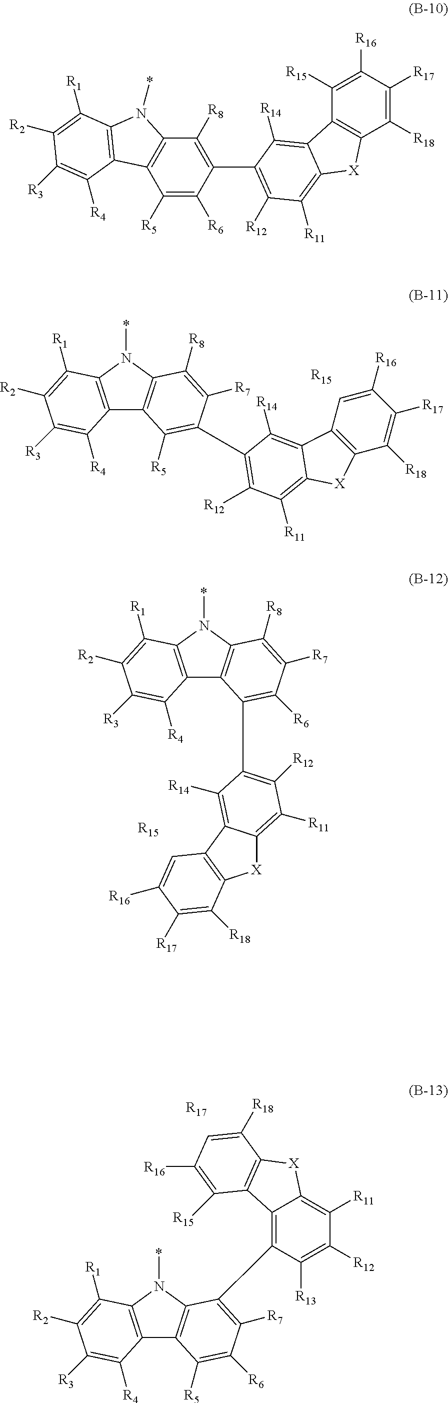

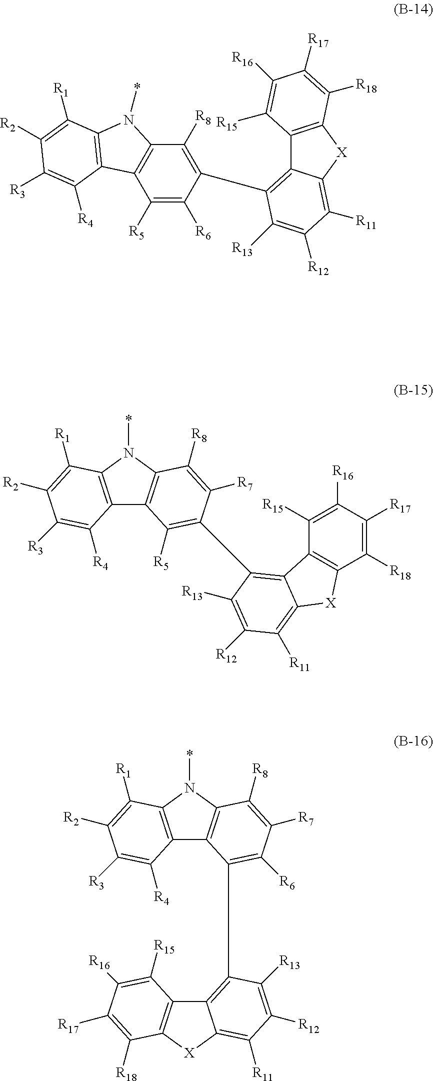

In formulas B-1 to B-16, R₁ to R₈, R₁₁ to R₁₈, and X are as defined in formula 1 above.

In formula 1′, the definitions and preferred embodiments of L₁, HAr, and X are as described in formula 1 above.

In formula 1′, R₁ to R₈, and R₁₁ to R₁₈, each independently, represent hydrogen, deuterium a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl. According to one embodiment of the present disclosure, R₁ to R₈, and R₁₁ to R₁₈, each independently, represent hydrogen, deuterium, or a substituted or unsubstituted (C6-C12)aryl. According to another embodiment of the present disclosure, R₁ to R₈, and R₁₁ to R₁₈, each independently, represent hydrogen, deuterium, or a (C6-C12)aryl unsubstituted or substituted with deuterium. For example, R₁ to R₈, and R₁₁ to R₁₈, each independently, represent hydrogen, deuterium, or a phenyl unsubstituted or substituted with deuterium.

In formula 1′, any one of R₅ to R₈ is linked to any one of R₁₁ to R₁₄ to form a single bond, with the proviso that at least five of R₁ to R₈ and R₁₁ to R₁₈ are deuterium.

Hereinafter, the compound represented by formula 2 or 2′ will be described in more detail.

In formulas 2 and 2′, A₁ and A₂, each independently, represent a substituted or unsubstituted (C6-C30)aryl, a substituted or unsubstituted dibenzofuranyl, a substituted or unsubstituted dibenzothiophenyl, or a substituted or unsubstituted carbazolyl. According to one embodiment of the present disclosure, A₁ and A₂, each independently, represent a substituted or unsubstituted (C6-C25)aryl, a substituted or unsubstituted dibenzofuranyl, a substituted or unsubstituted dibenzothiophenyl, or a substituted or unsubstituted carbazolyl. The substituent of the substituted (C6-C25)aryl may be at least one of a (C1-C6)alkyl(s); a (C6-C20)aryl(s); a (5- to 15-membered)heteroaryl(s) unsubstituted or substituted with a (C6-C20)aryl(s); and a tri(C6-C12)arylsilyl(s). The substituent of the substituted dibenzofuranyl, the substituted dibenzothiophenyl, and the substituted carbazolyl, each independently, may be a (C6-C12)aryl(s). According to another embodiment of the present disclosure, A₁ and A₂, each independently, represent a substituted or unsubstituted, phenyl, biphenyl, terphenyl, naphthyl, fluorenyl, benzofluorenyl, triphenylenyl, fluoranthenyl, phenanthrenyl, dibenzofuranyl, carbazolyl or dibenzothiophenyl. For example, A₁ and A₂, each independently, may be a phenyl, a naphthyl, a biphenyl, a terphenyl, a triphenylenyl, a naphthylphenyl, a phenylnaphthyl, a phenyl substituted with a triphenylenyl(s), a naphthylphenyl, a phenyl substituted with a methyl(s), a phenyl substituted with a pyridyl(s), a phenyl substituted with a phenylpyridyl(s), a phenyl substituted with a dibenzofuranyl(s), a phenyl substituted with a dibenzothiophenyl(s), a phenyl substituted with a triphenylsilyl(s), a diphenylfluorenyl, a dimethylfluorenyl, a dimethylbenzofluorenyl, a dibenzofuranyl, a dibenzothiophenyl, a dibenzofuranyl substituted with a phenyl(s), a dibenzothiophenyl substituted with a phenyl(s), a carbazolyl substituted with a phenyl(s), or a carbazolyl substituted with a naphthyl(s), which may be further substituted with deuterium.

In formulas 2 and 2′. X₁₁ to X₂₆, each independently, represent hydrogen, deuterium, a substituted or unsubstituted (C6-C30)aryl, or a substituted or unsubstituted (3- to 30-membered)heteroaryl, or may be linked to an adjacent substituent(s) to form a ring(s). According to one embodiment of the present disclosure, X₁₁ to X₂₆, each independently, represent hydrogen, deuterium, a substituted or unsubstituted (C6-C12)aryl, or a substituted or unsubstituted (5- to 15-membered)heteroaryl, or may be linked to an adjacent substituent(s) to form a substituted or unsubstituted mono- or polycyclic (3- to 30-membered) alicyclic or aromatic ring, or the combination thereof. According to another embodiment of the present disclosure, X₁₁ to X₂₆, each independently, represent hydrogen, deuterium, a (C6-C12)aryl unsubstituted or substituted with deuterium, or a (5- to 15-membered)heteroaryl unsubstituted or substituted with deuterium, or may be linked to an adjacent substituent(s) to form a substituted or unsubstituted monocyclic (3- to 10-membered) aromatic ring. For example, X₁₁ to X₂₆, each independently, may be hydrogen, deuterium, a phenyl unsubstituted or substituted with deuterium, a dibenzofuranyl unsubstituted or substituted with deuterium, or a dibenzothiophenyl unsubstituted or substituted with deuterium, or any two adjacent ones of X₁₁ to X₂₆ may be linked to each other to form a benzene ring. In formulas 2 and 2′, any one of X₁₁ to X₁₈ is linked to any one of X₁₉ to X₂₆ to form a single bond.

In formulas 2 and 2′, at least four of X₁₁ to X₂₆ are deuterium, and at least one of X₁₁, X₁₈, X₁₉, and X₂₆ is deuterium.

According to one embodiment of the present disclosure, formula 2 or 2′ is represented by at least one of the following formulas 2-1 to 2-8.

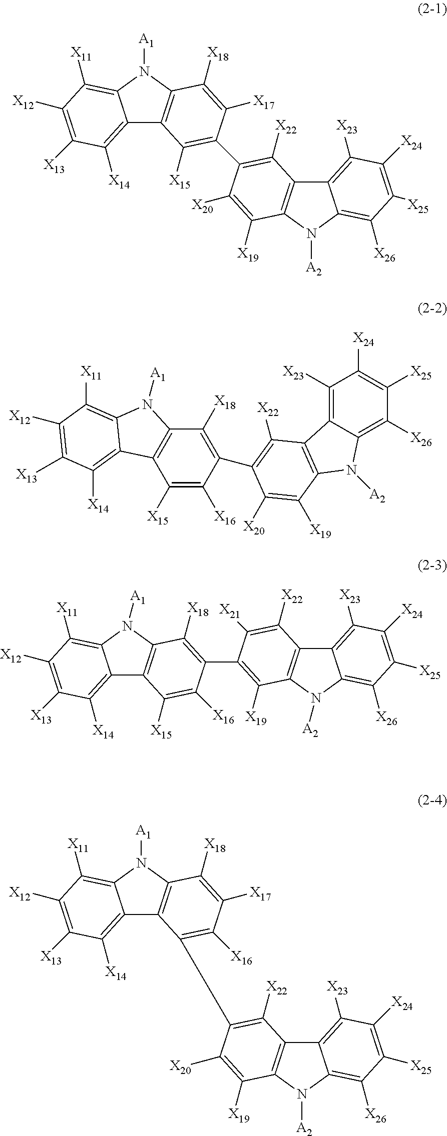

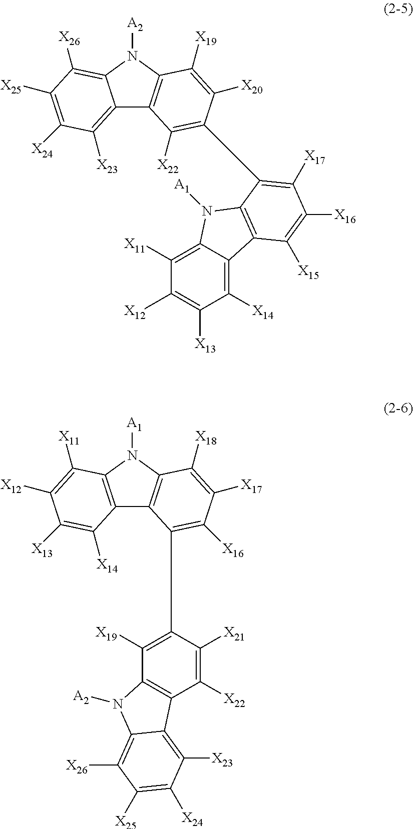

In formulas 2-1 to 2-8, A₁, A₂, and X₁₁ to X₂₆ are as defined in formula 2 or 2′.

The compound represented by formula 1 may be at least one selected from the following compounds, but is not limited thereto.

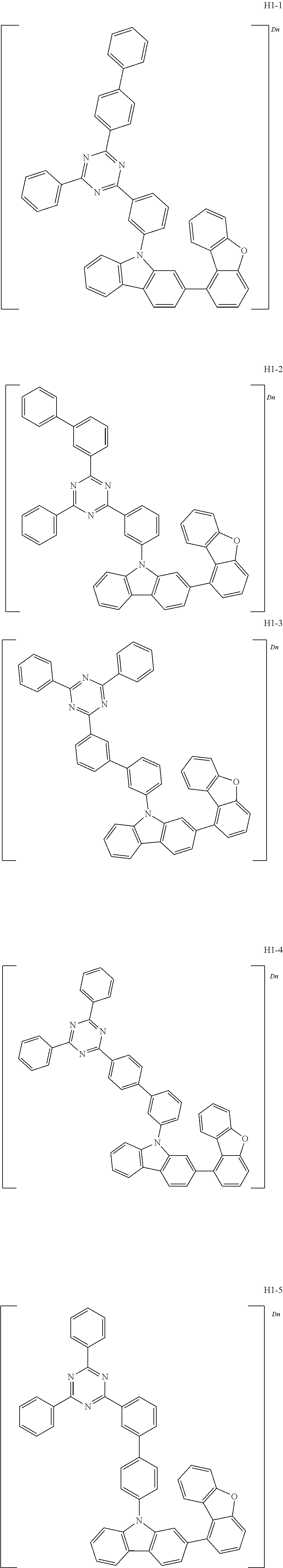

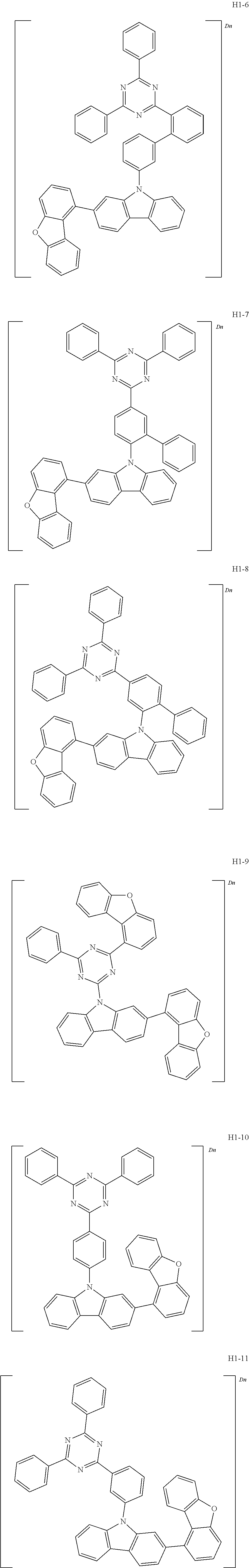

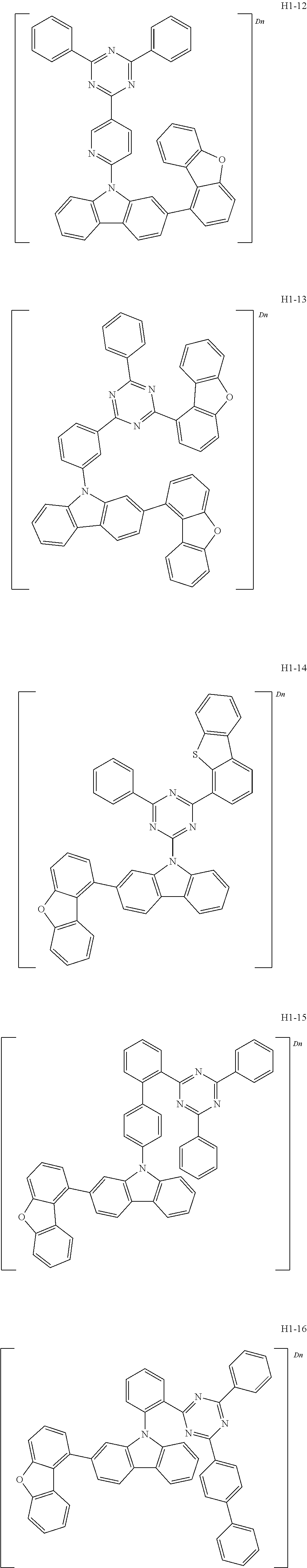

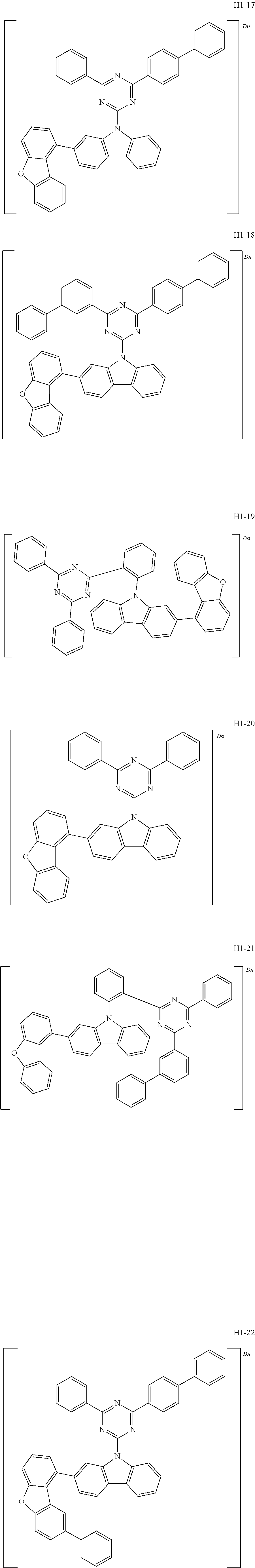

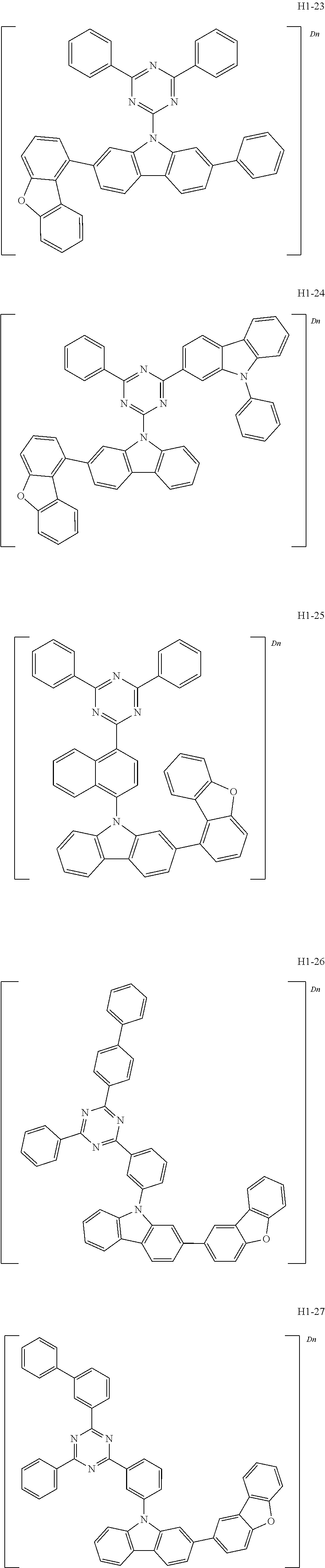

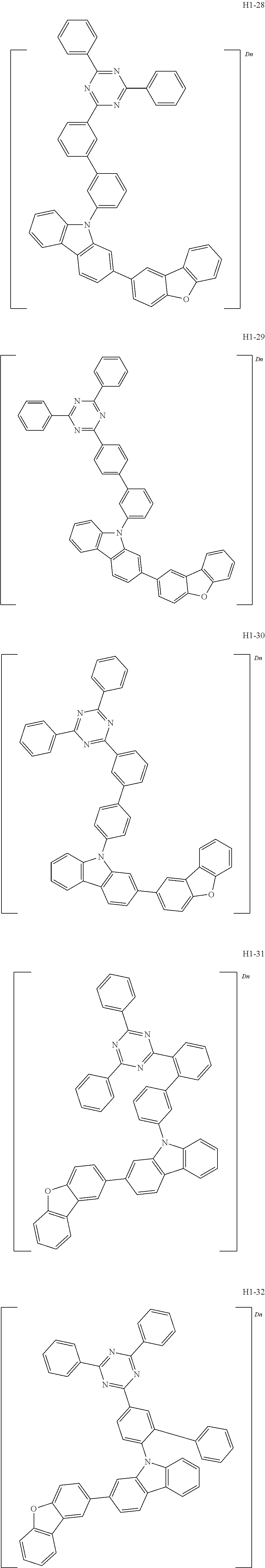

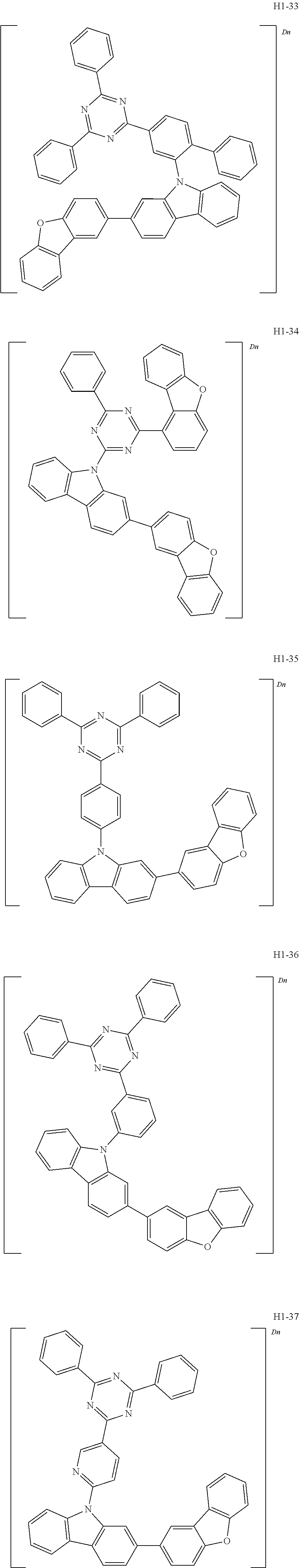

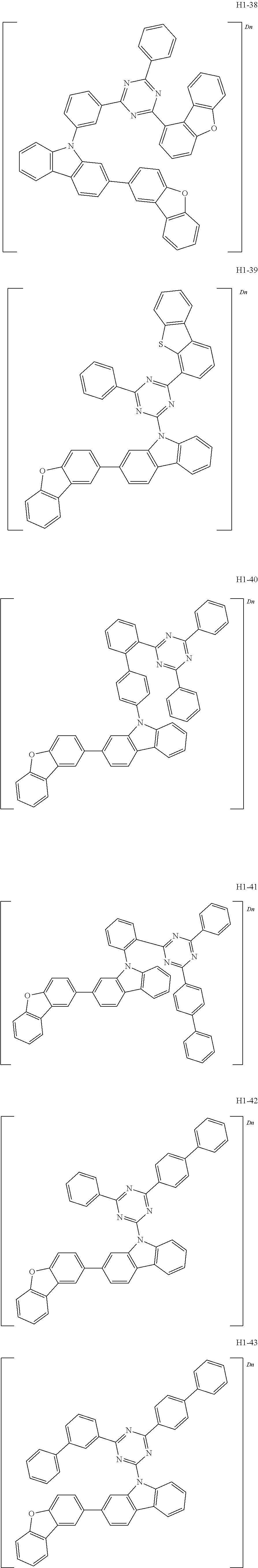

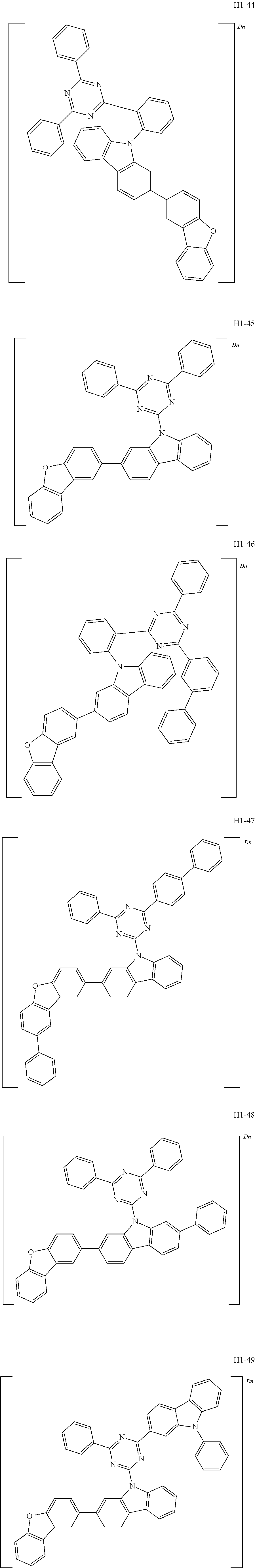

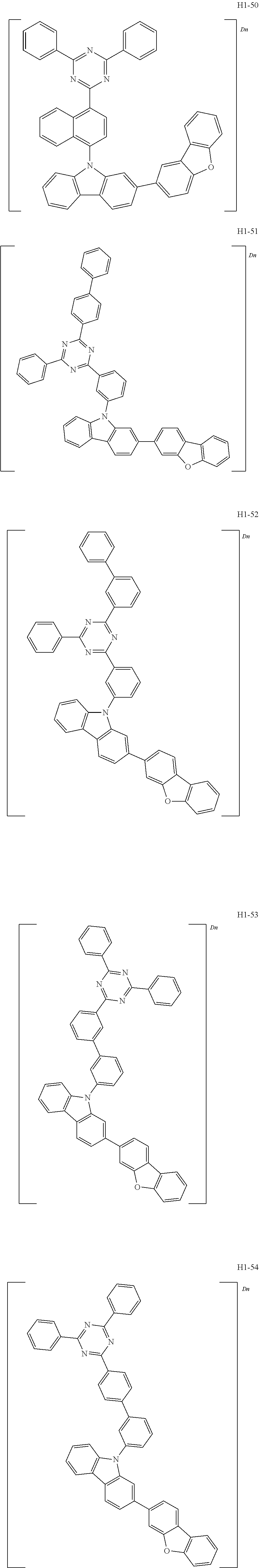

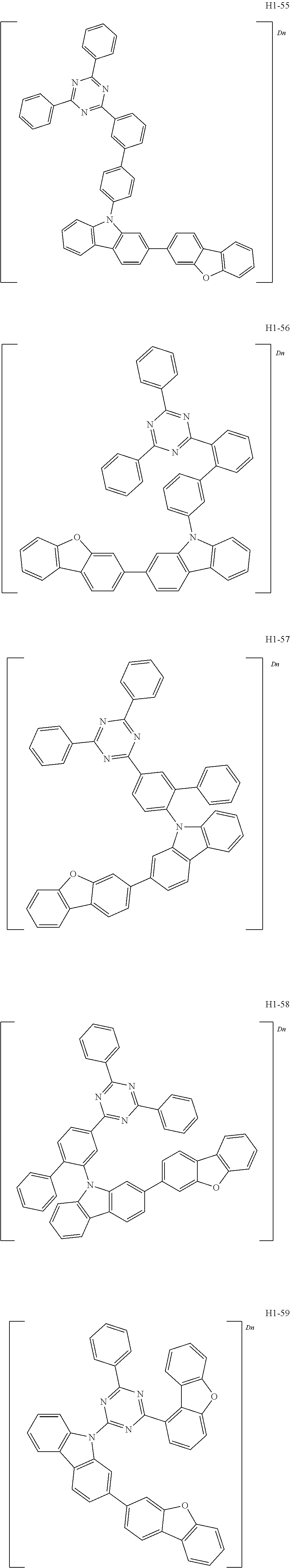

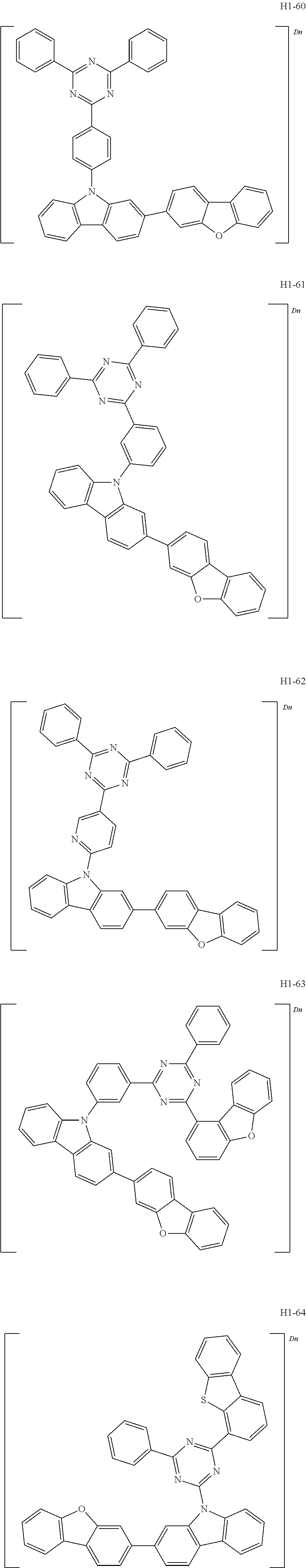

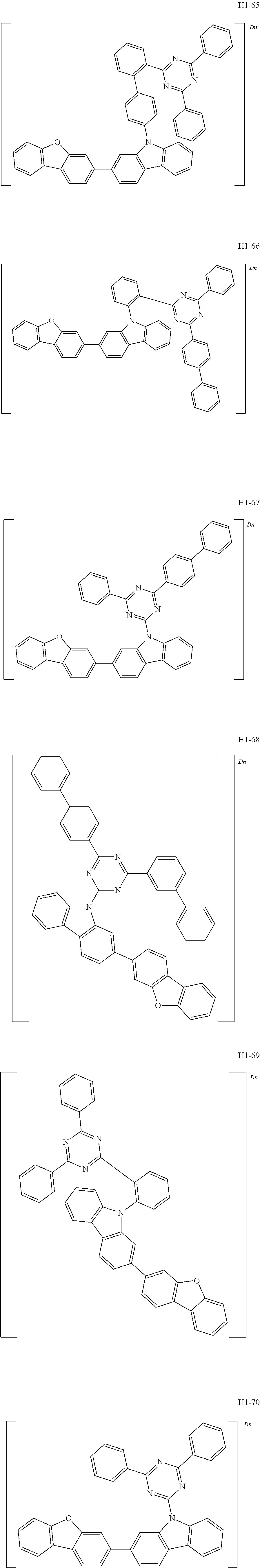

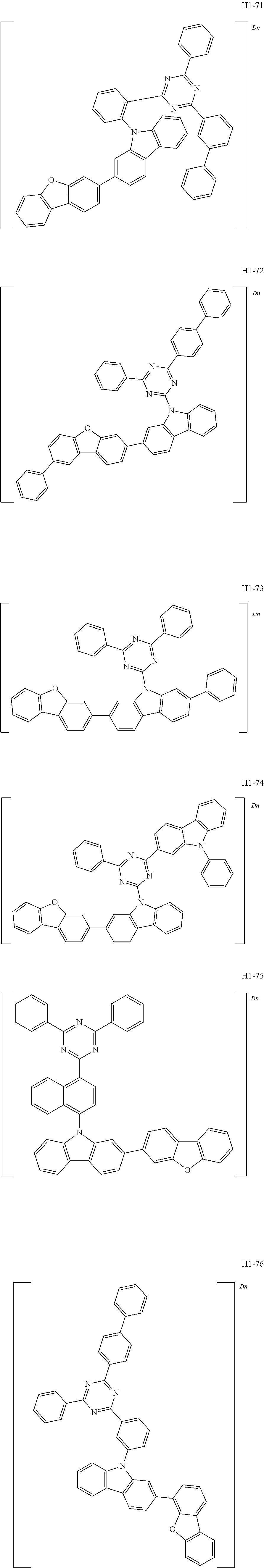

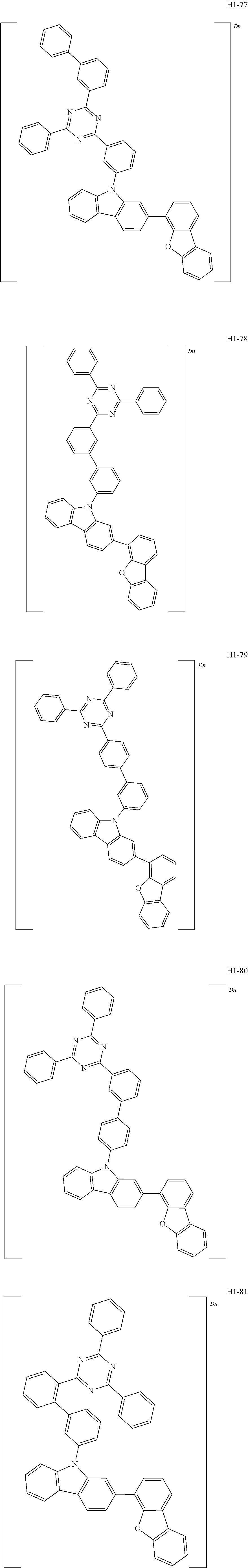

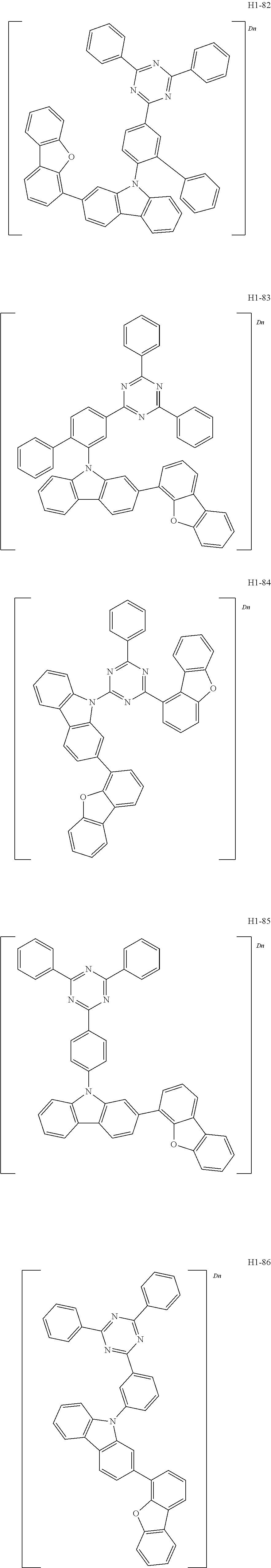

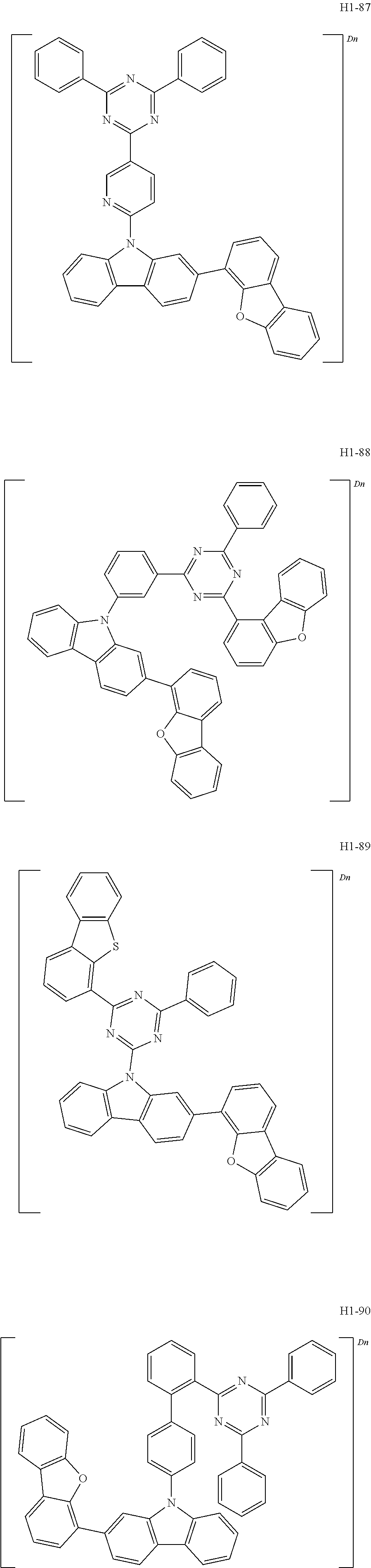

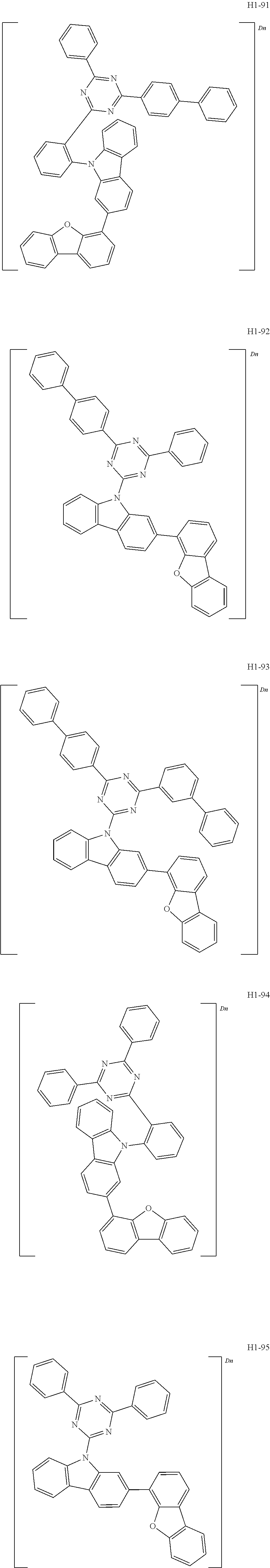

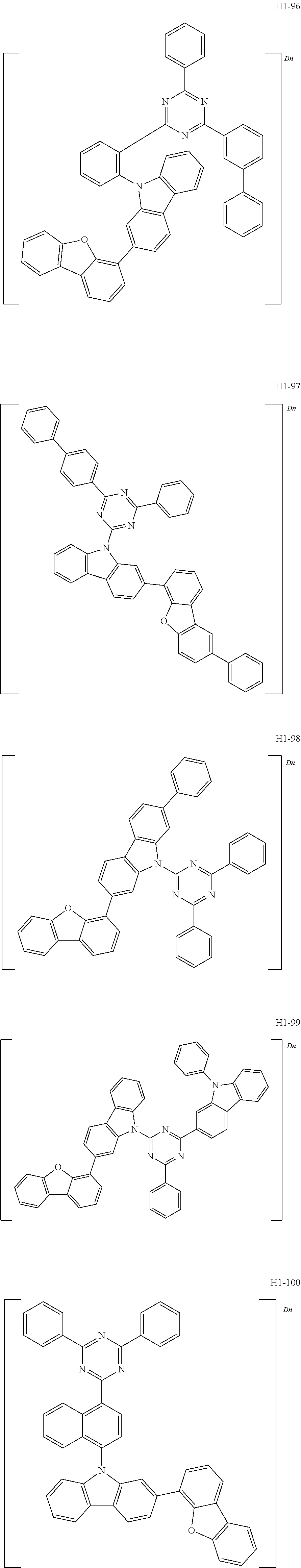

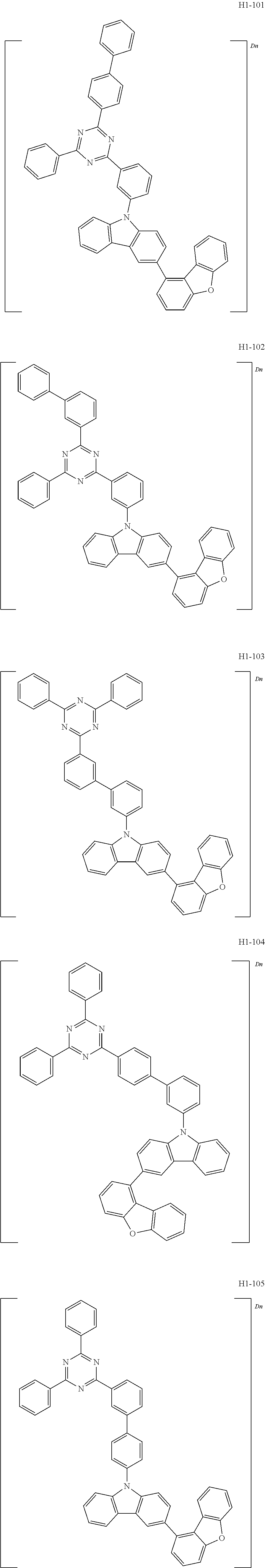

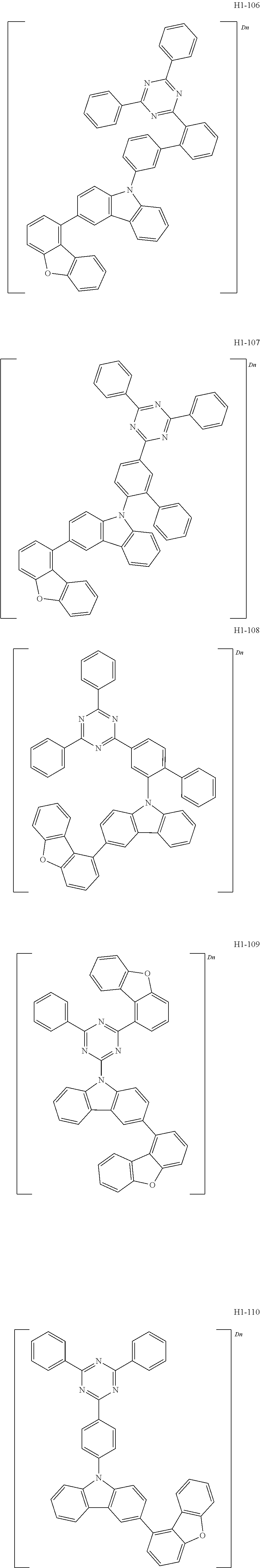

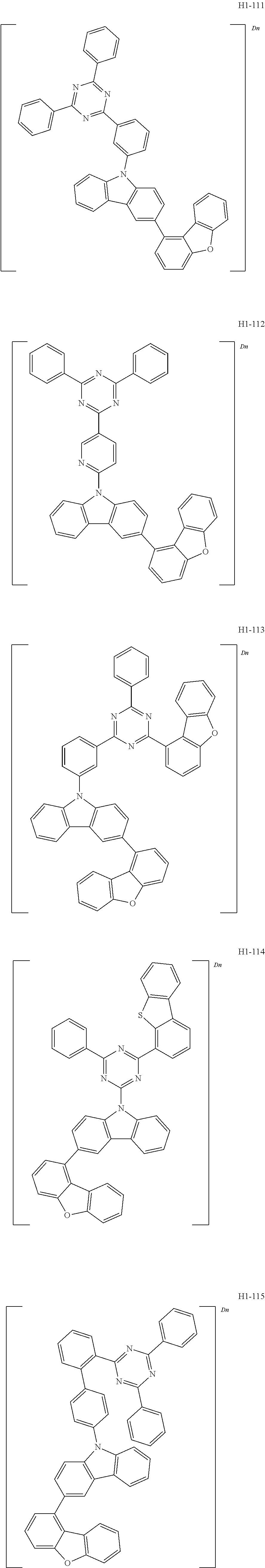

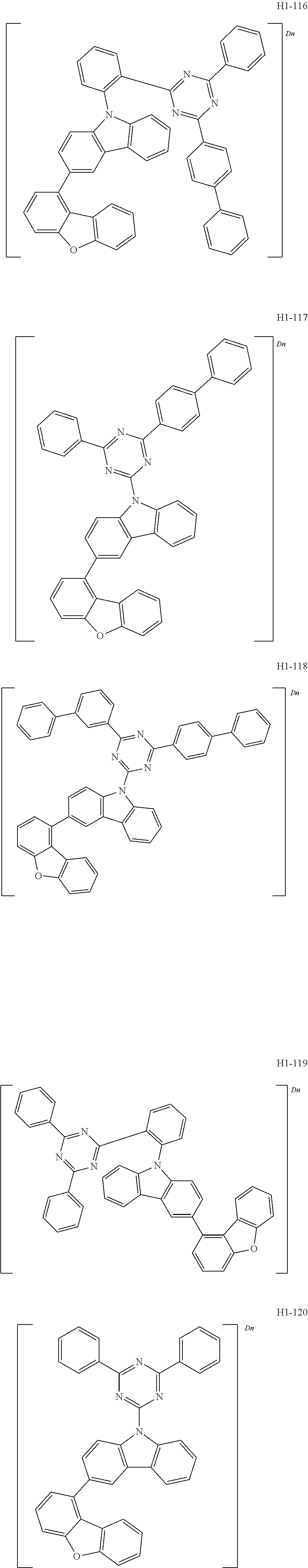

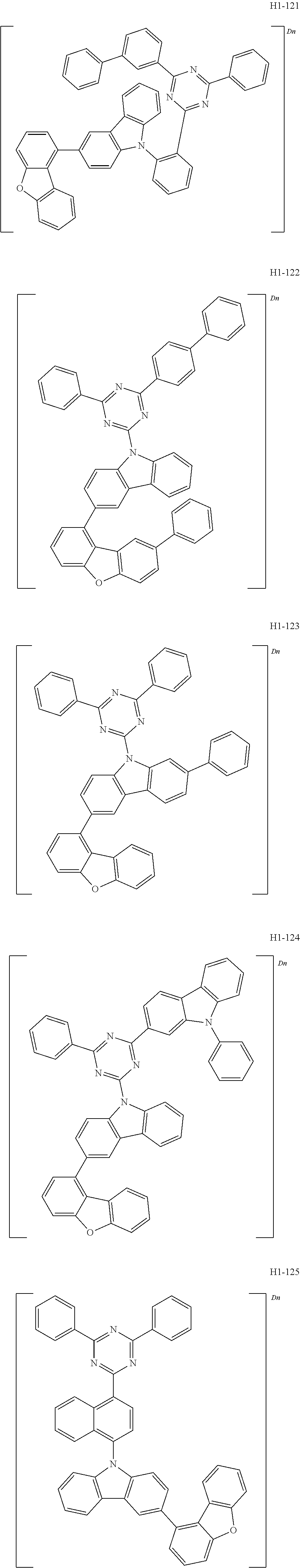

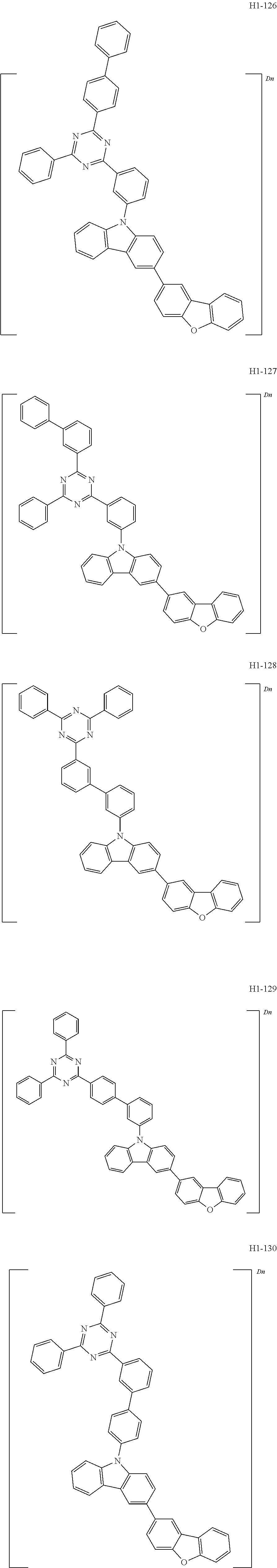

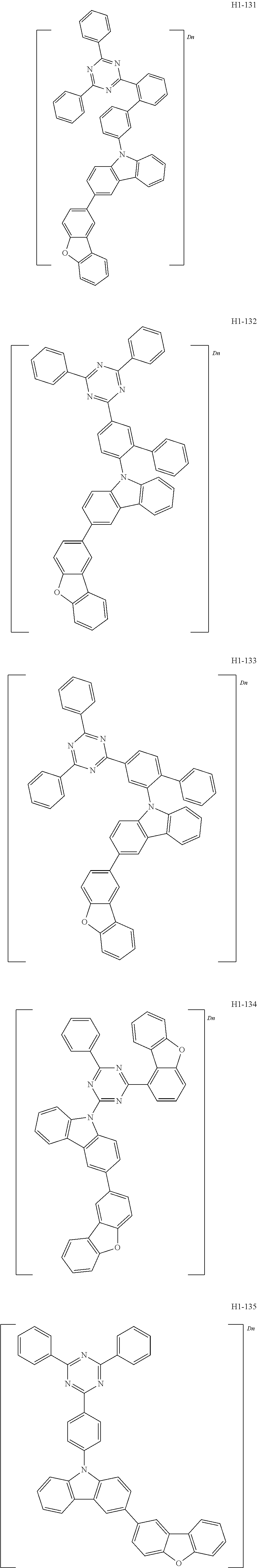

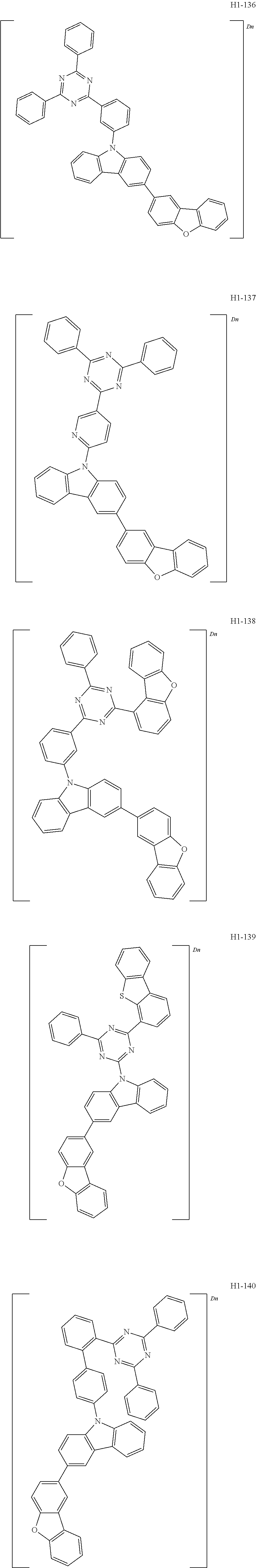

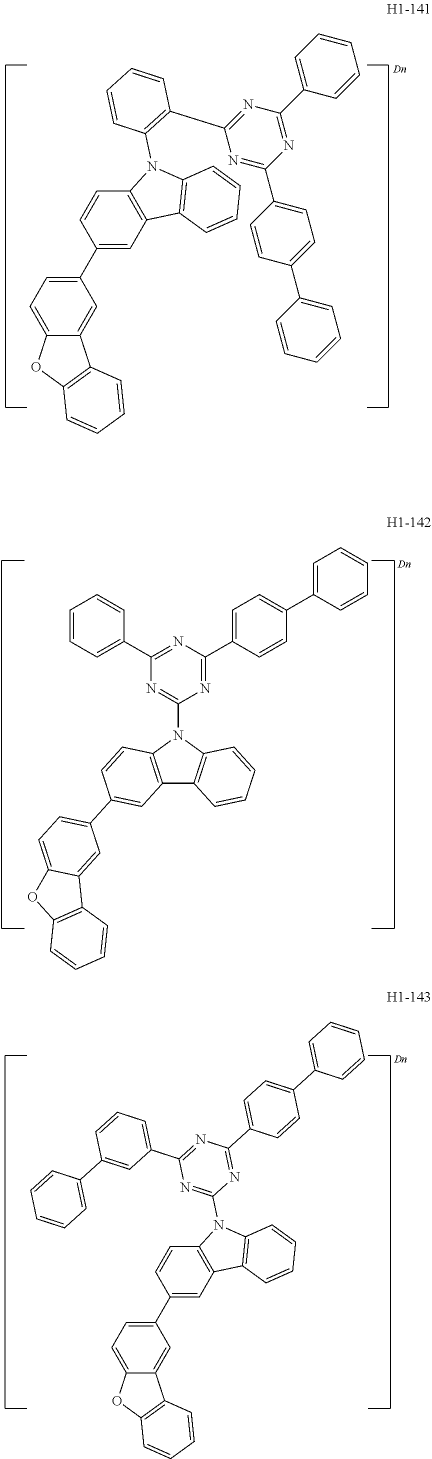

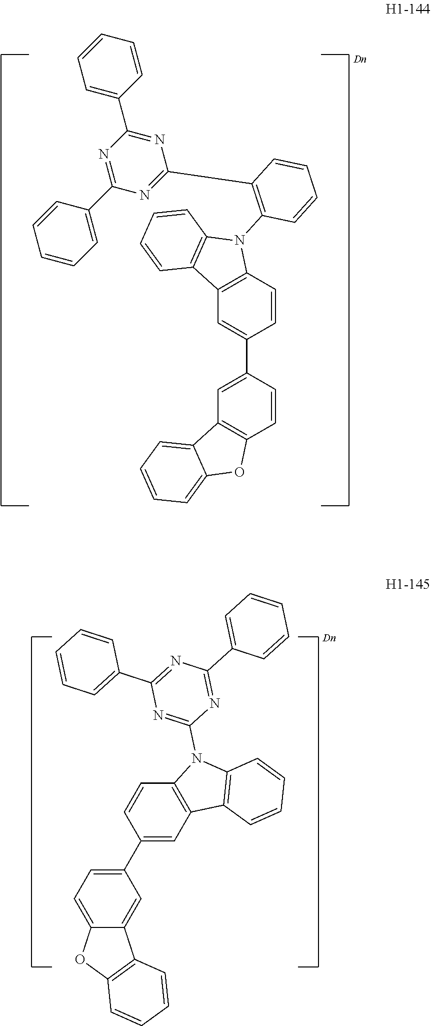

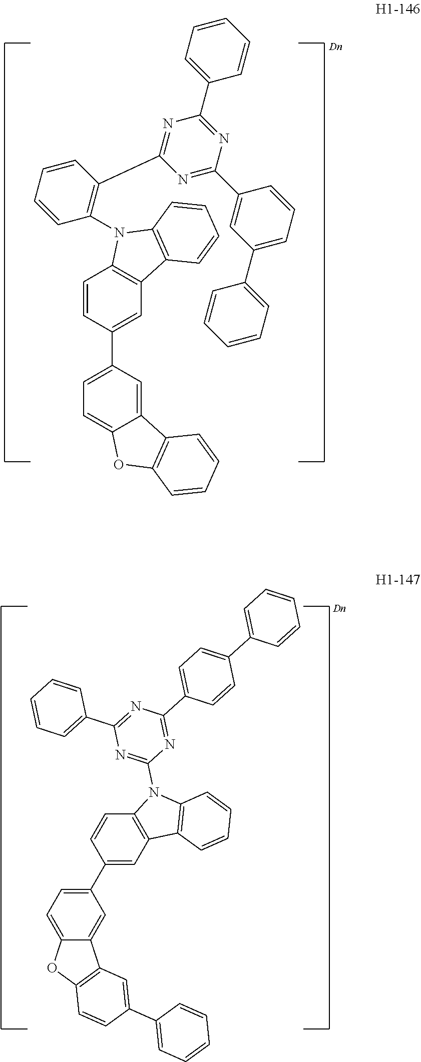

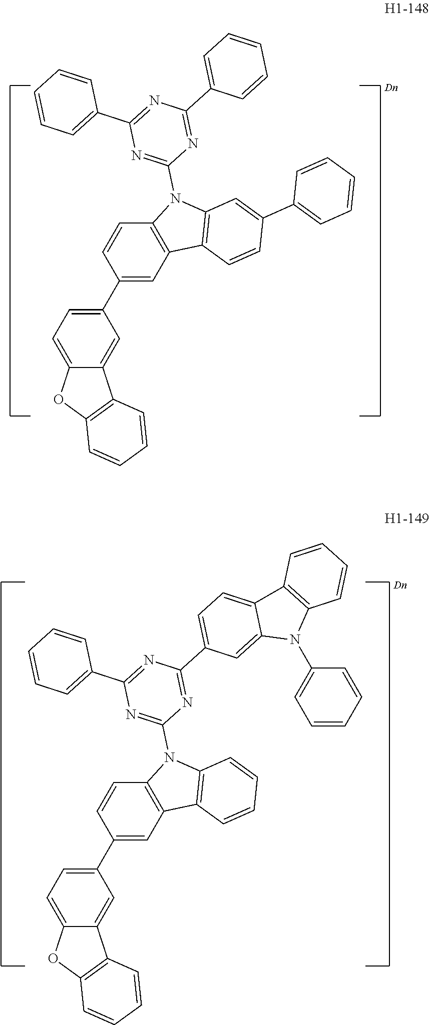

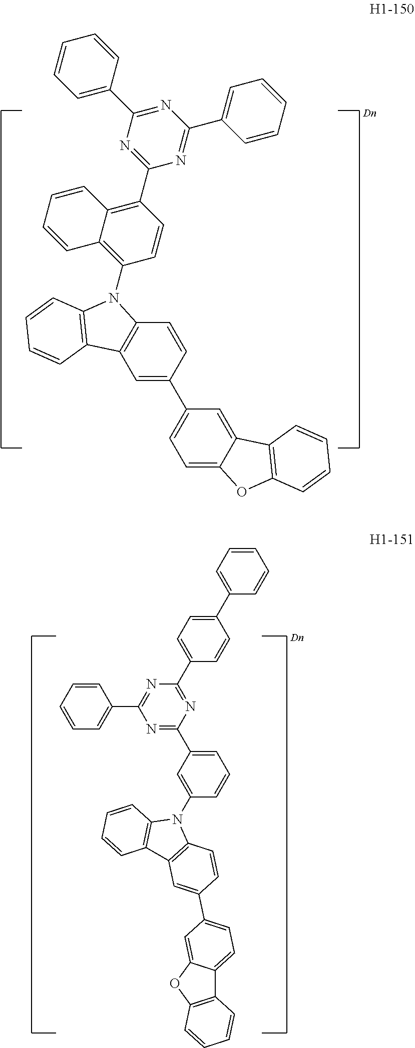

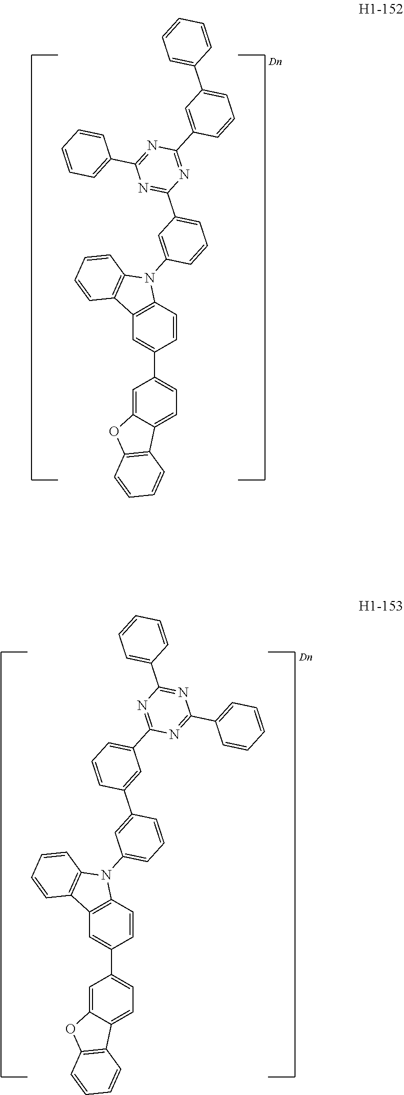

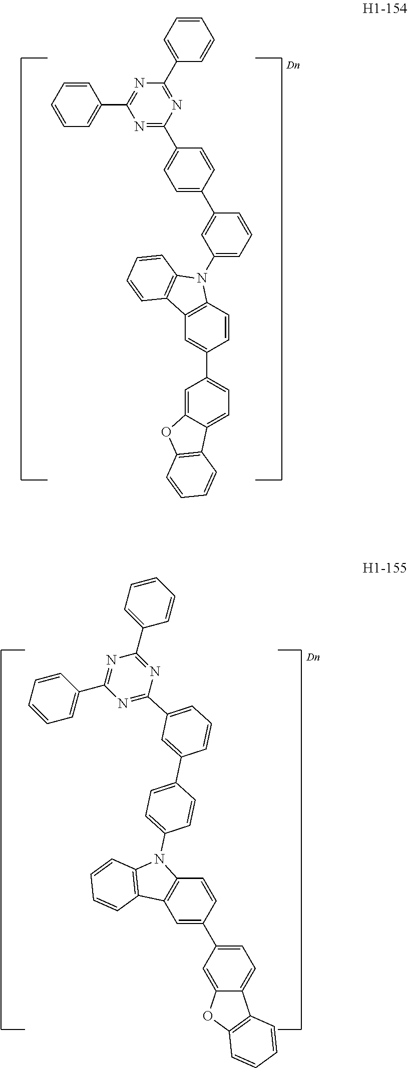

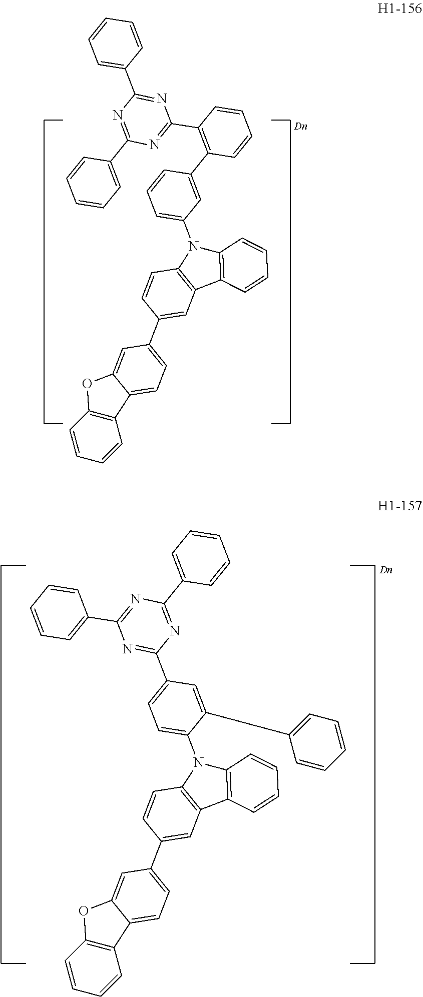

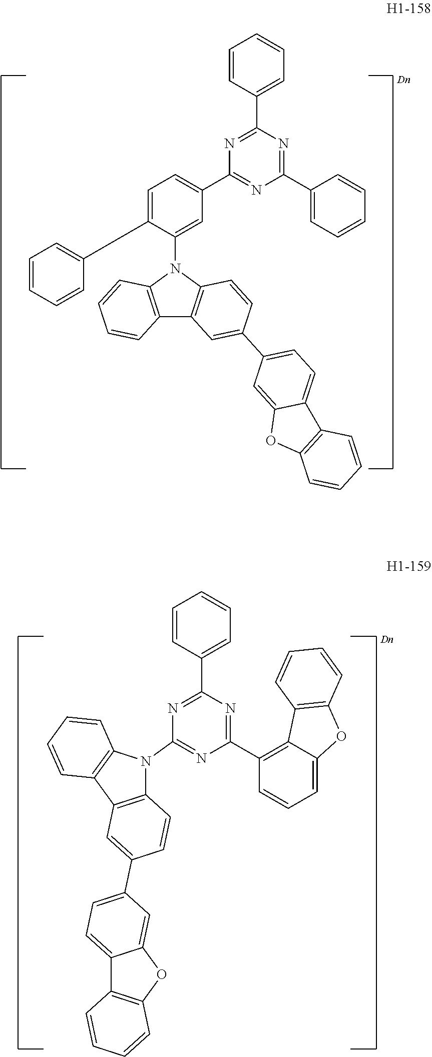

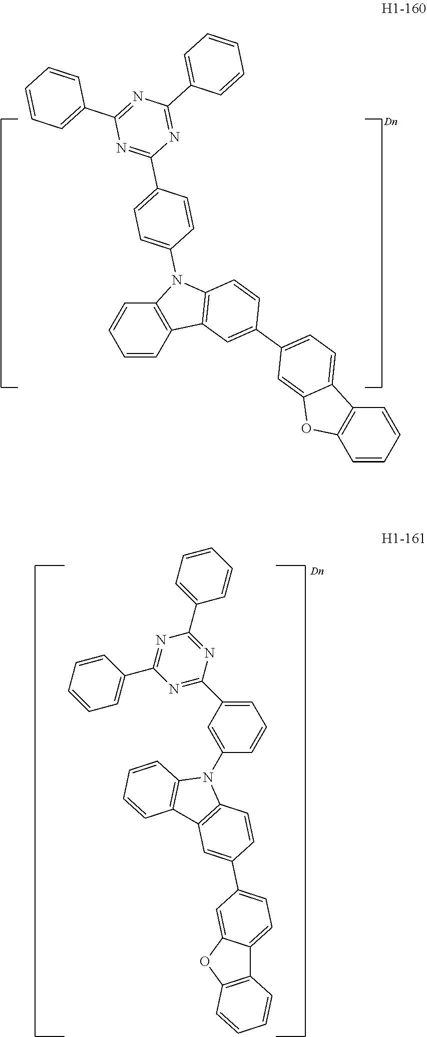

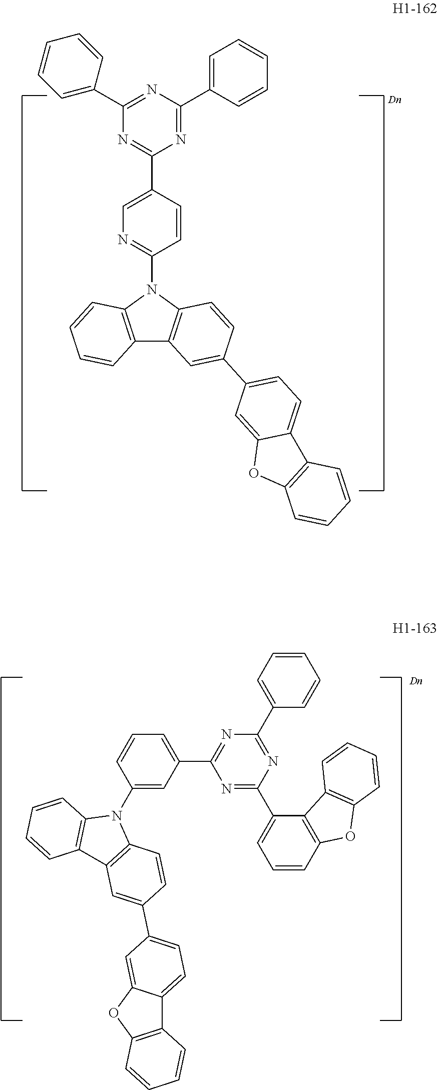

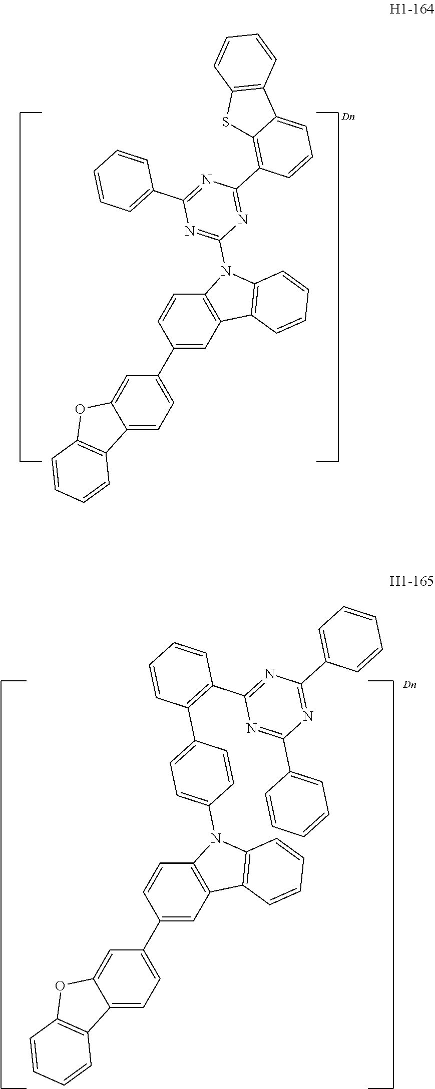

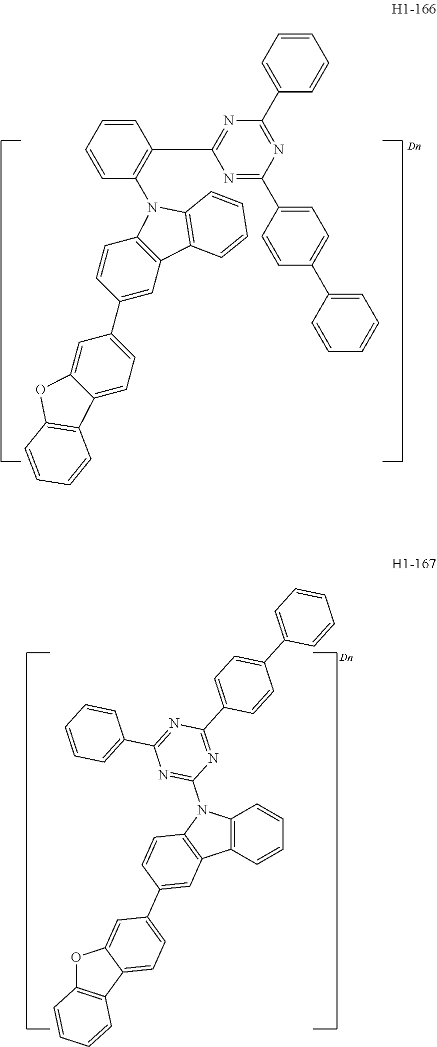

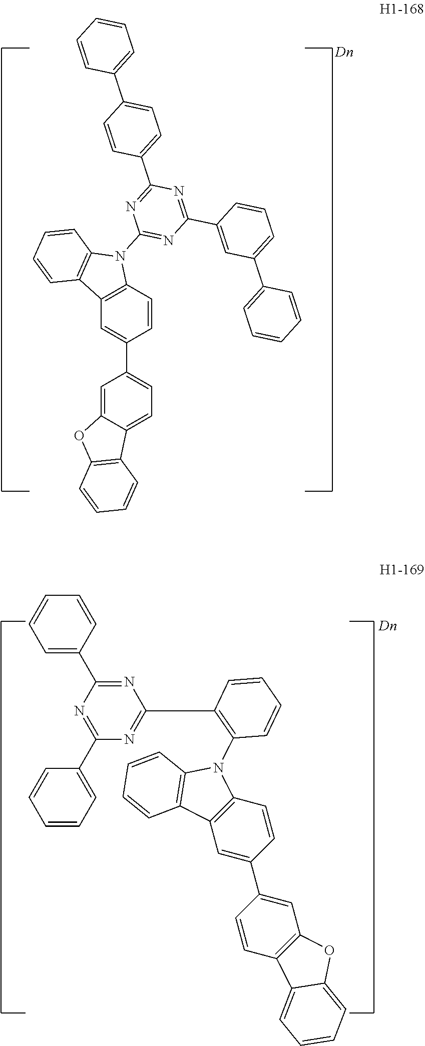

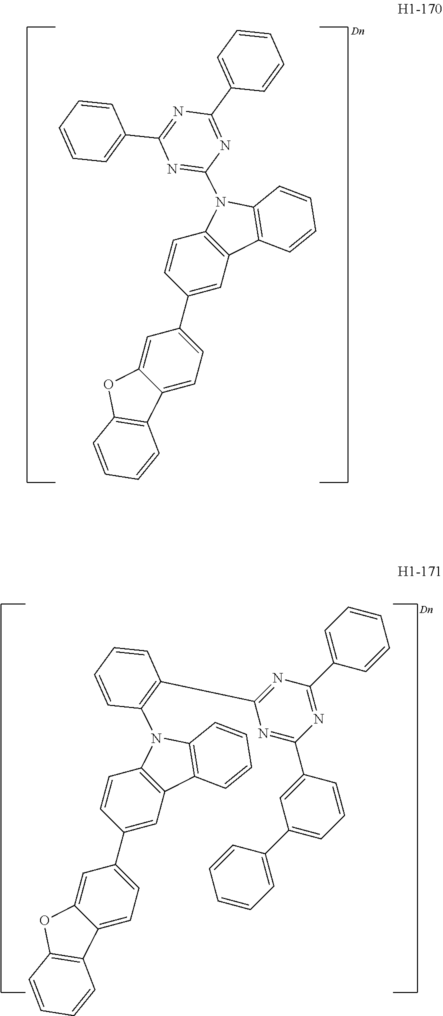

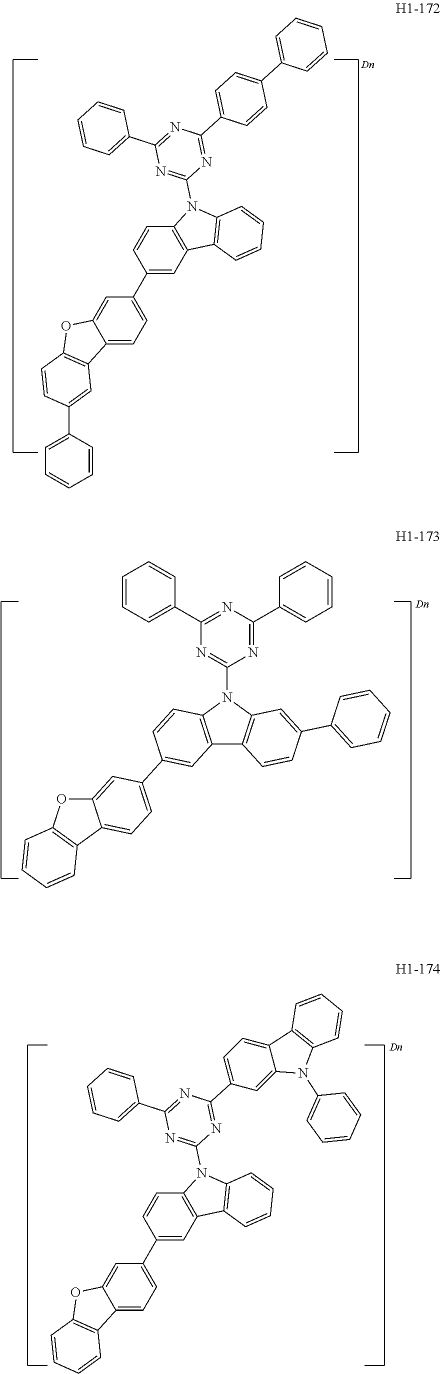

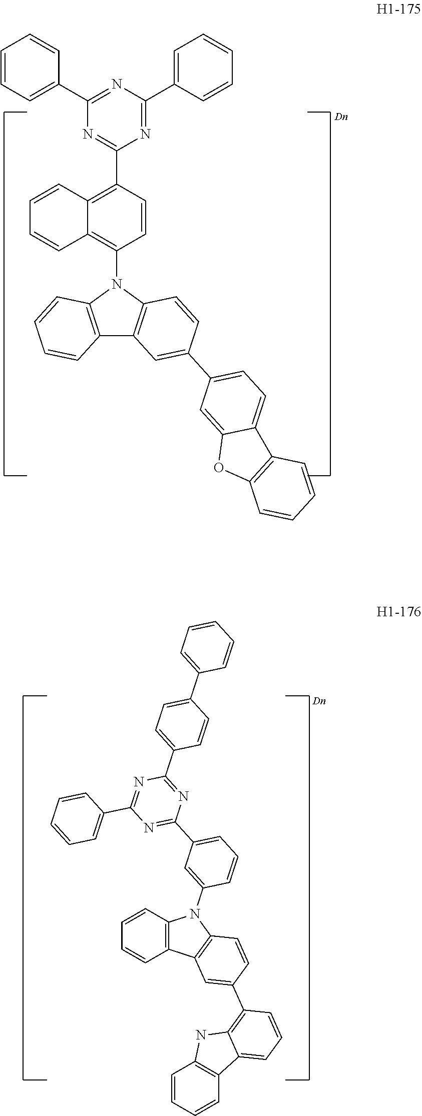

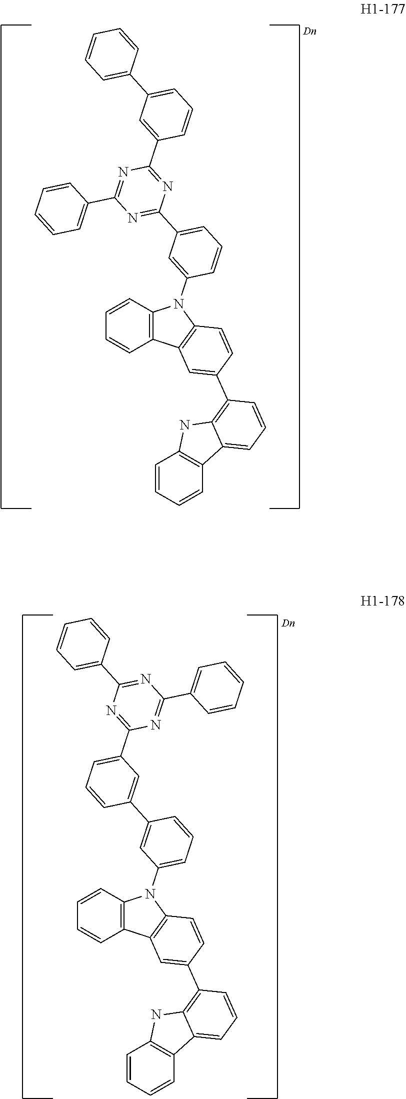

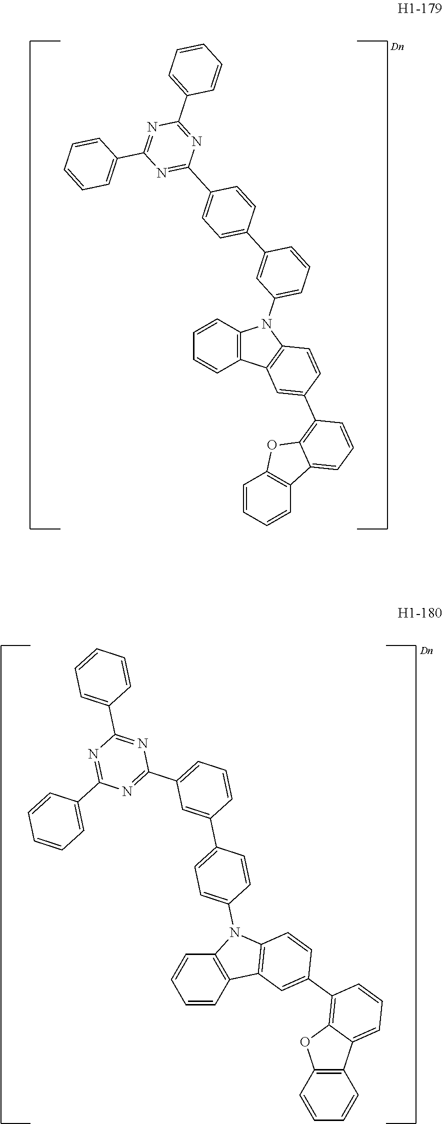

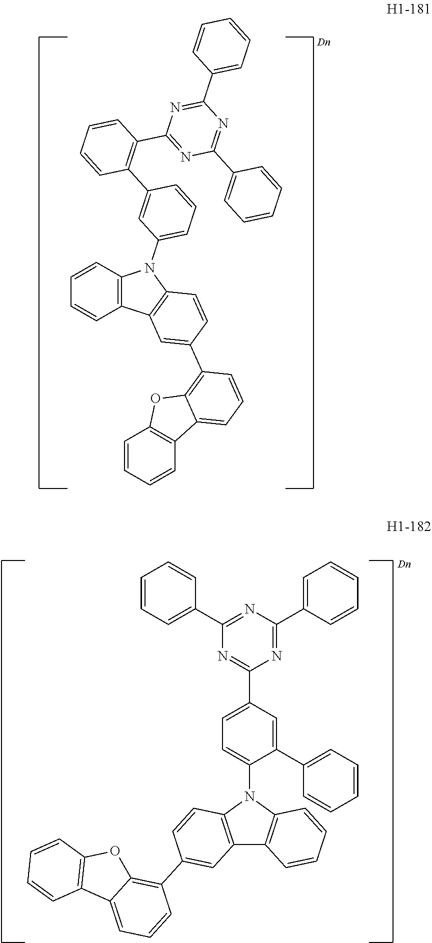

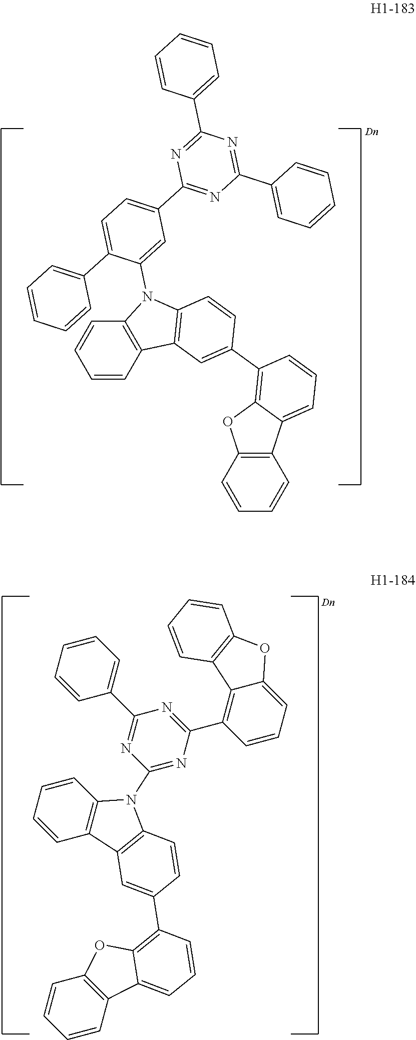

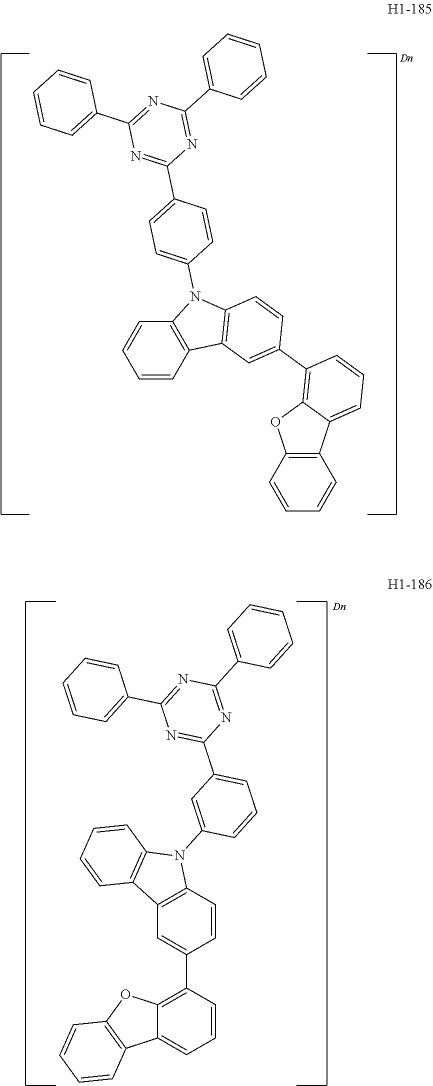

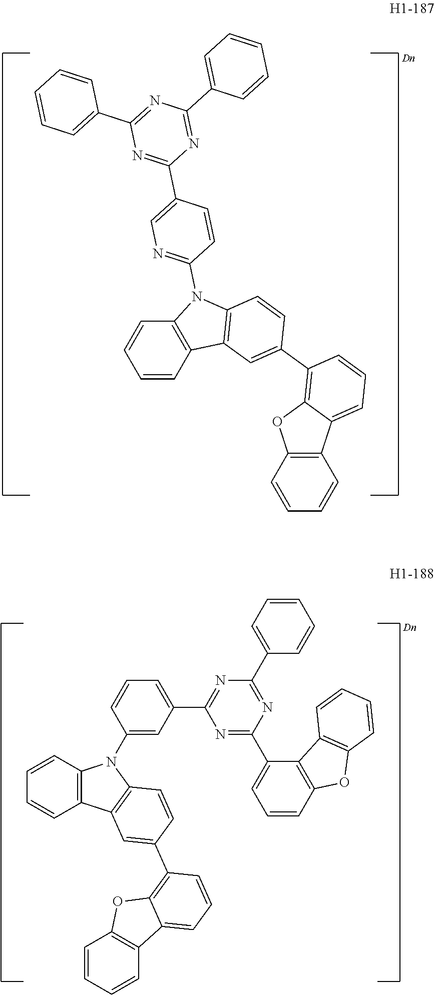

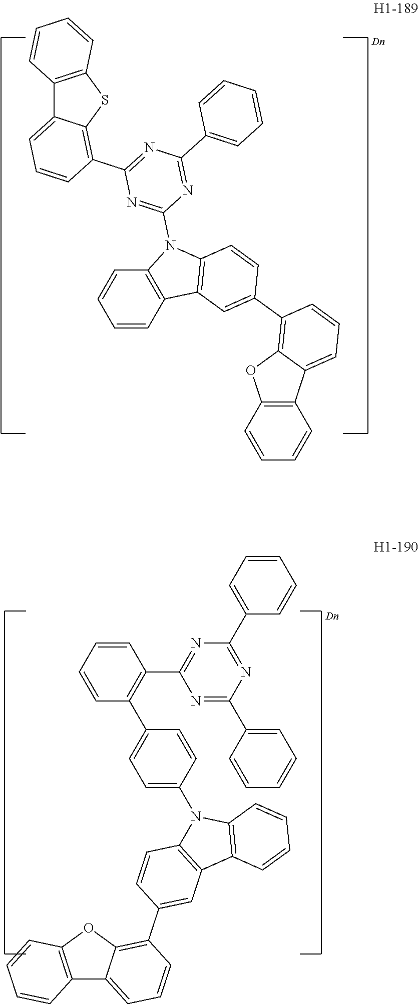

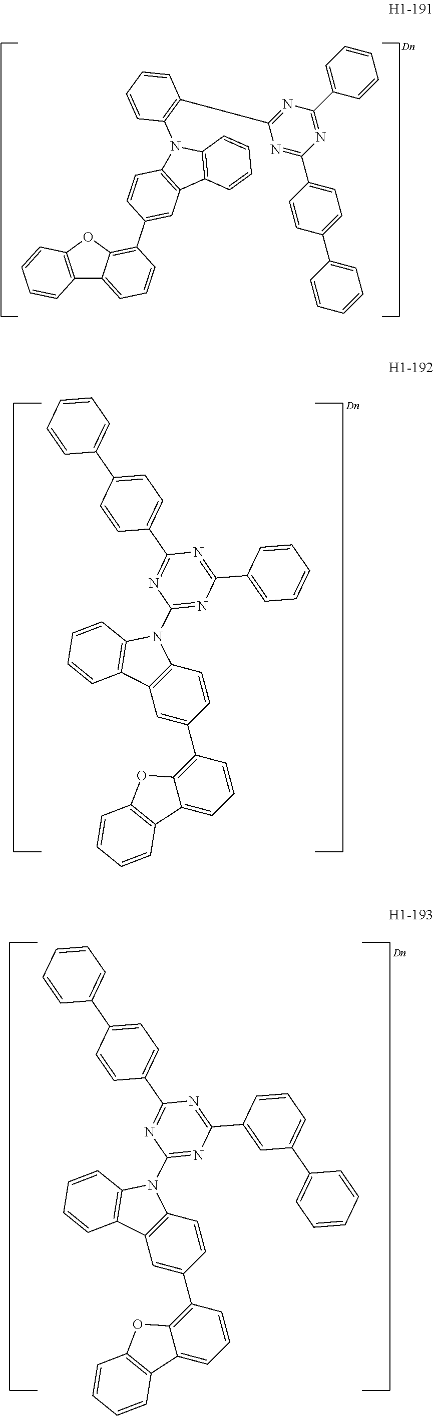

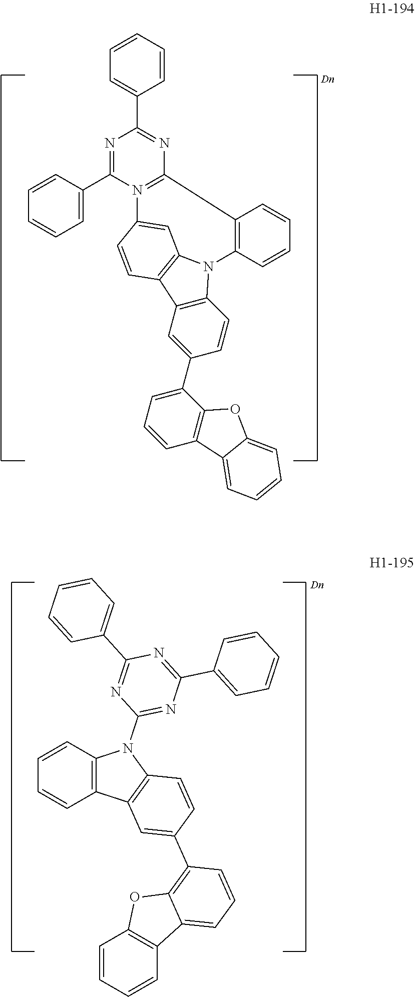

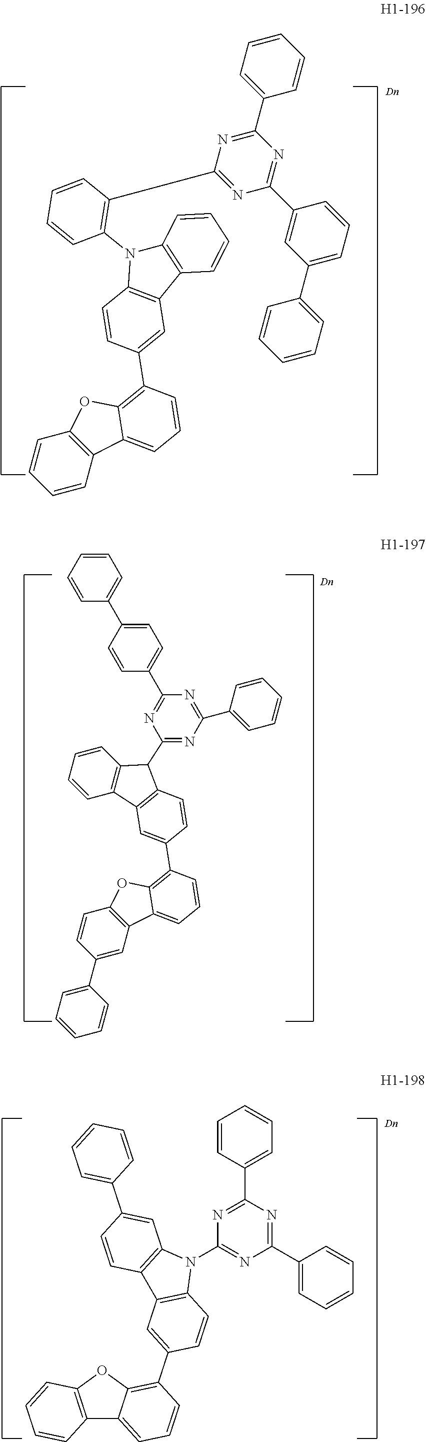

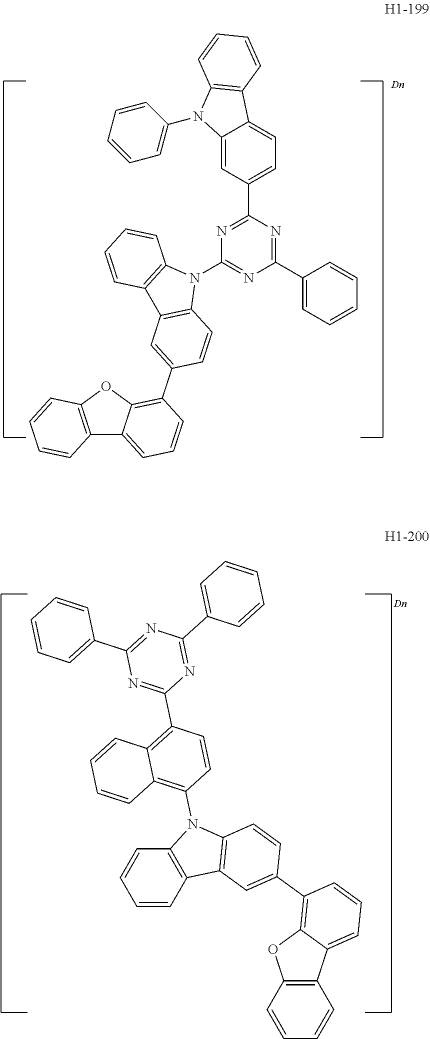

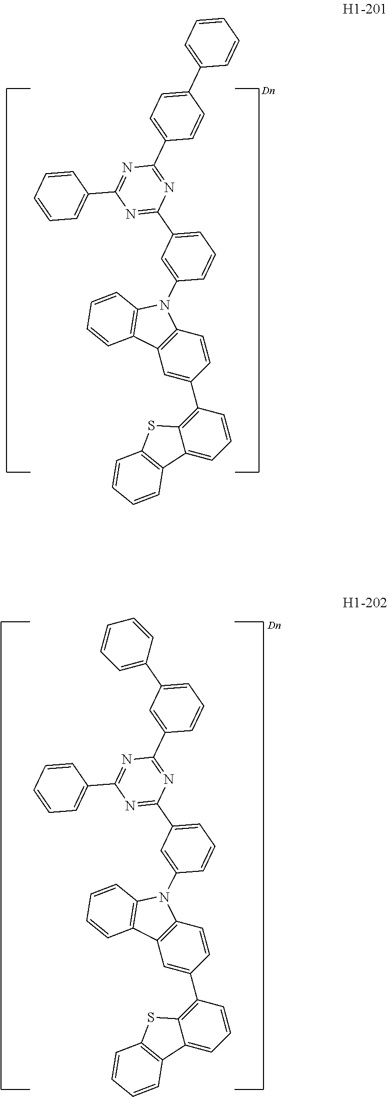

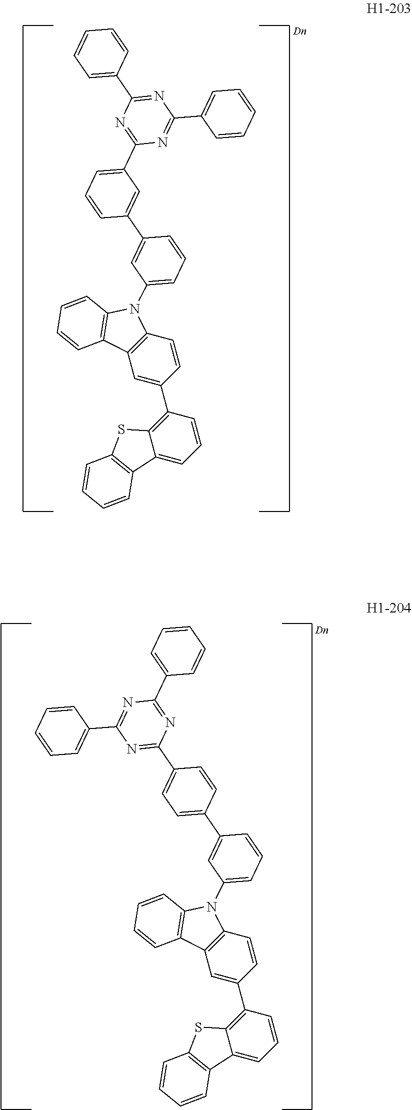

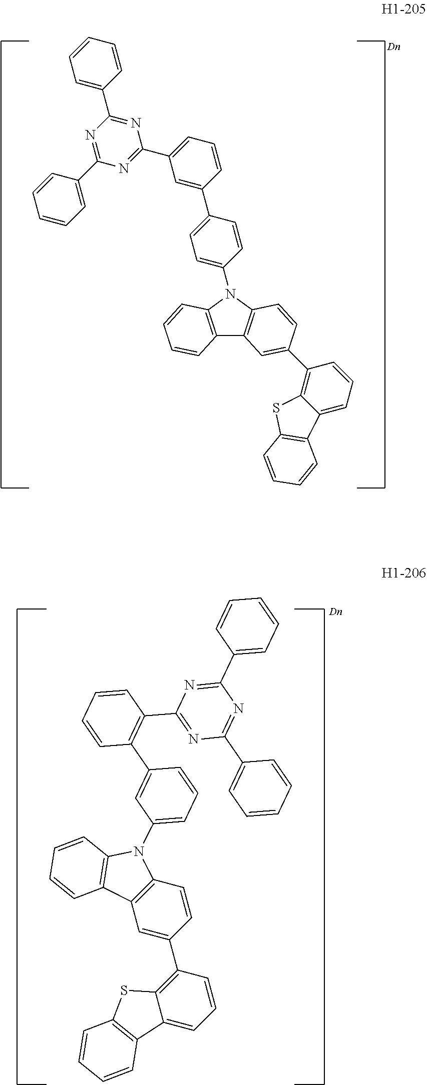

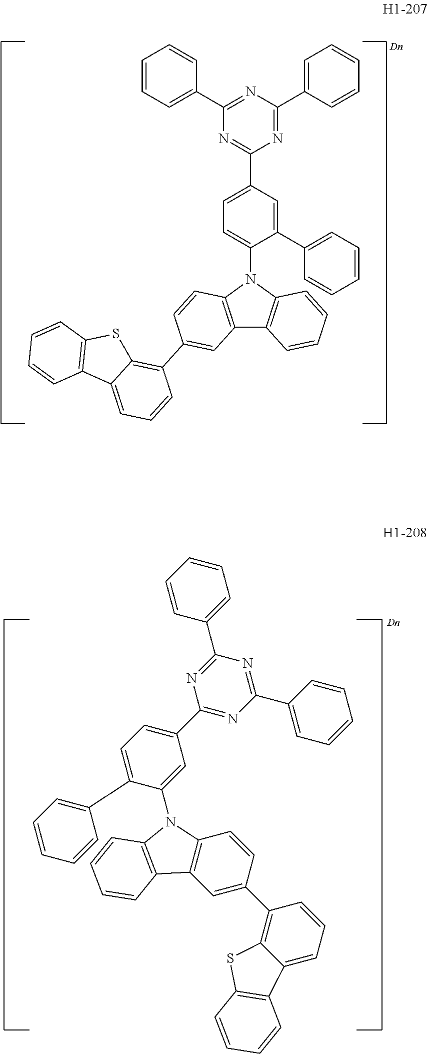

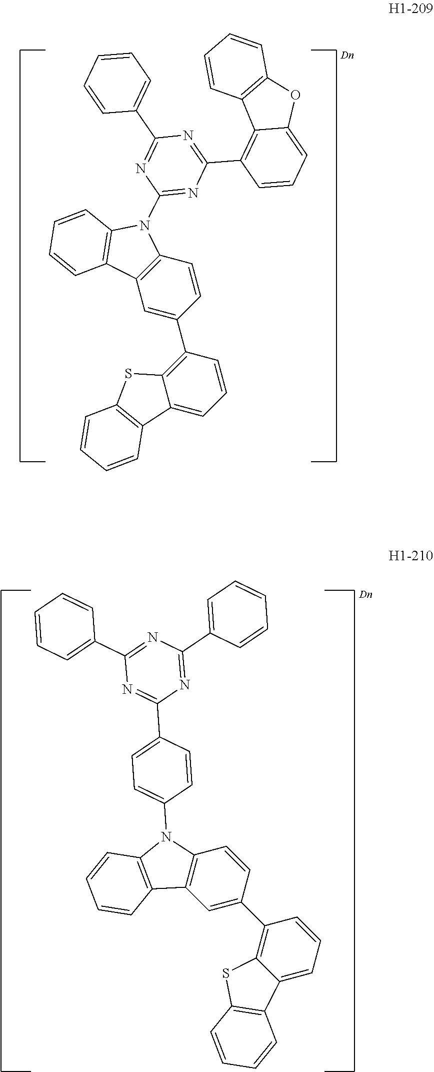

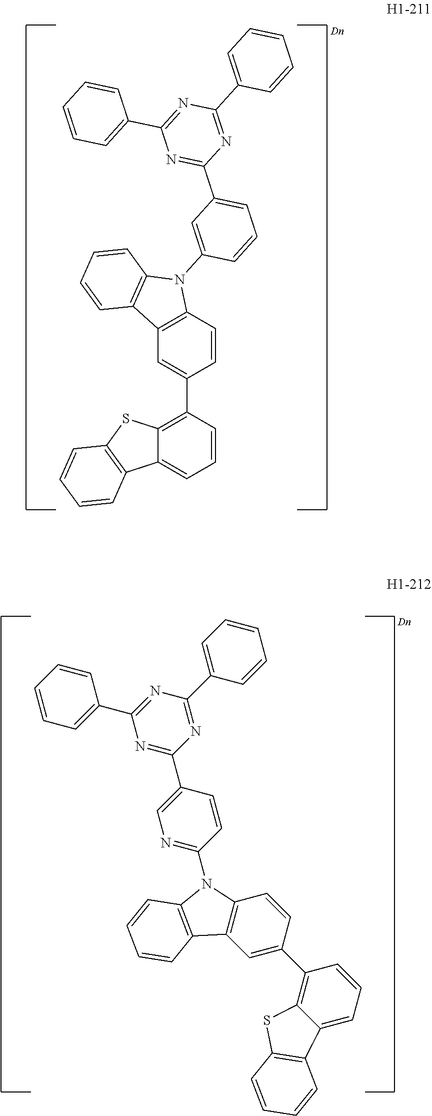

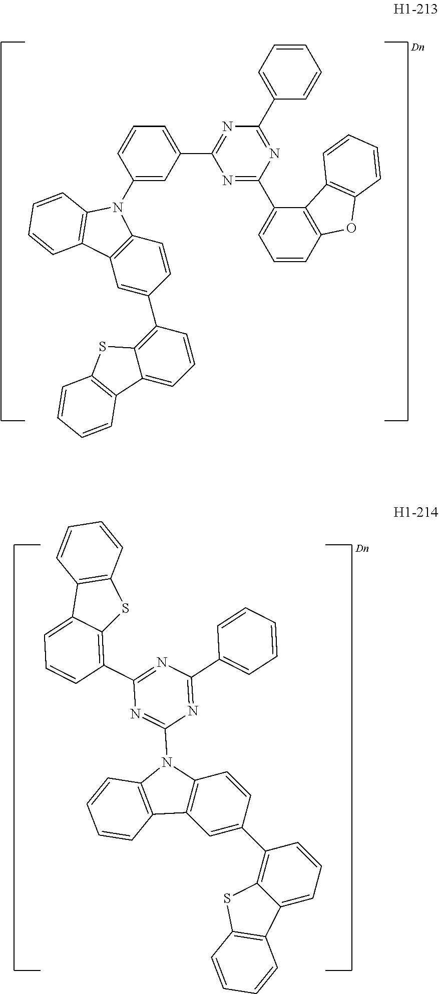

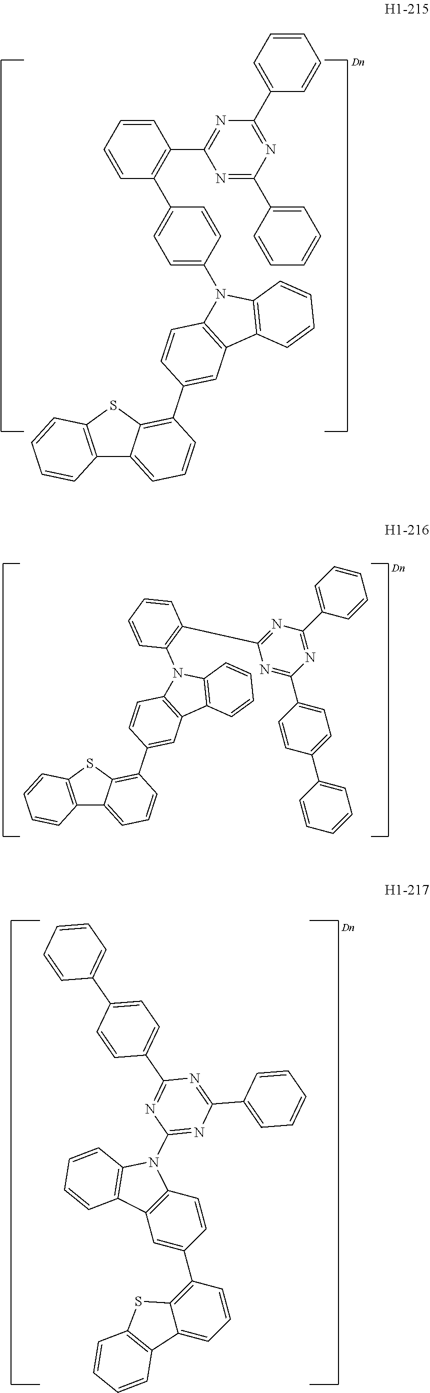

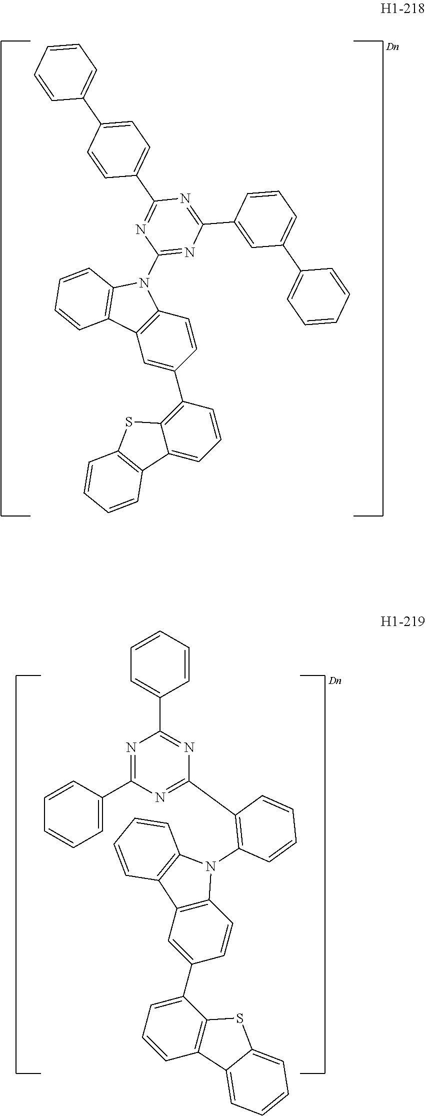

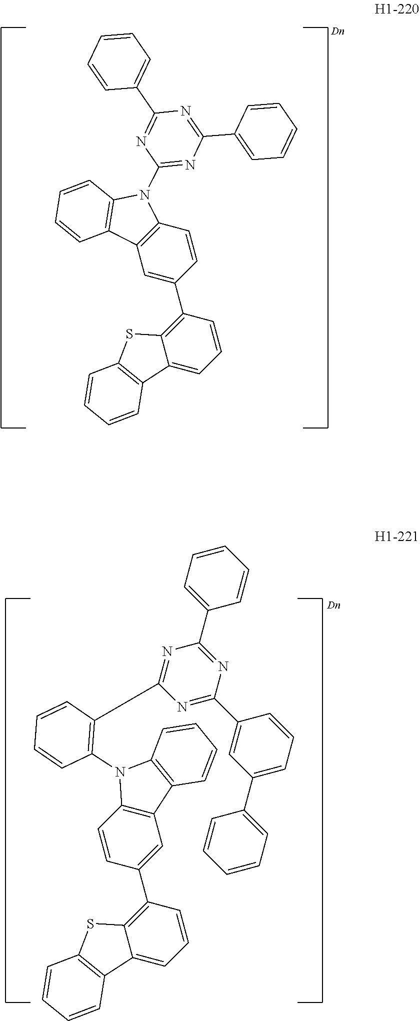

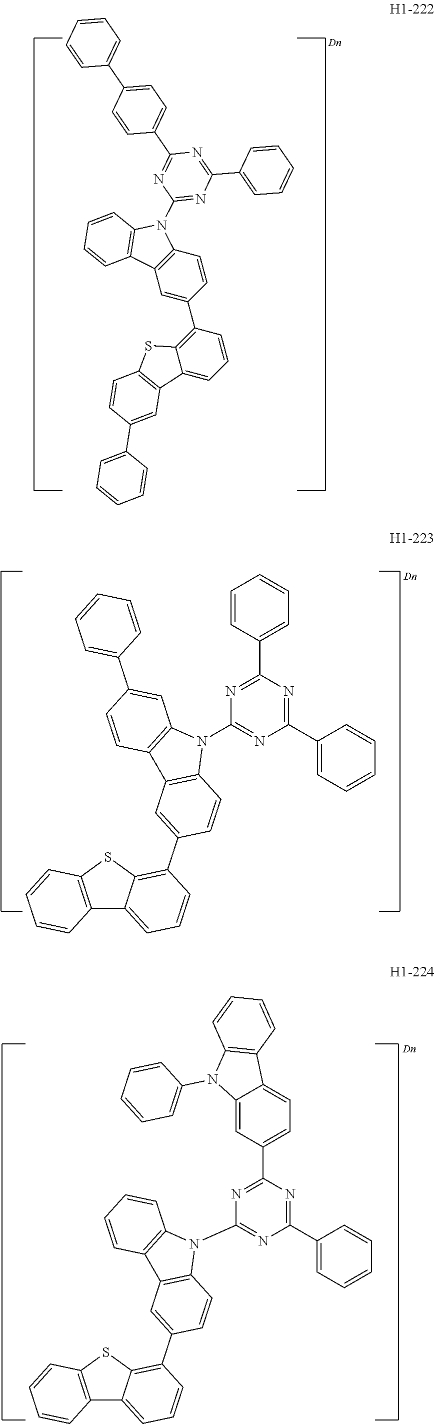

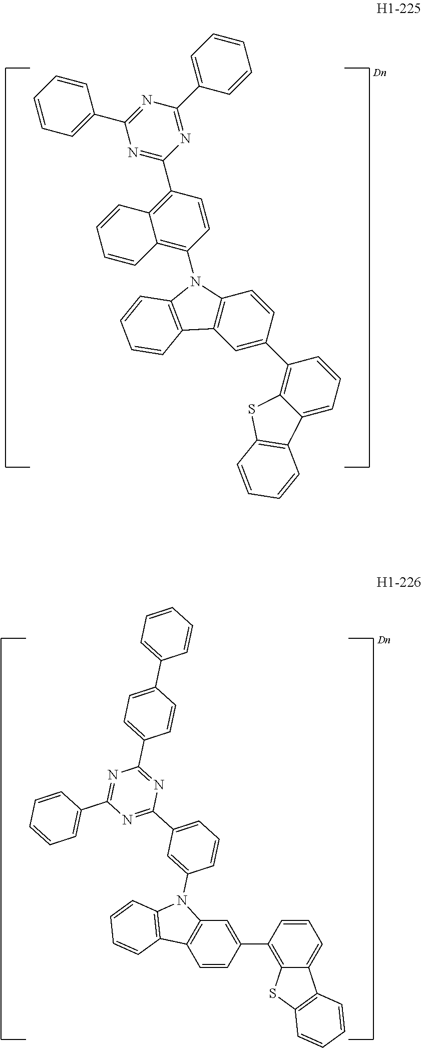

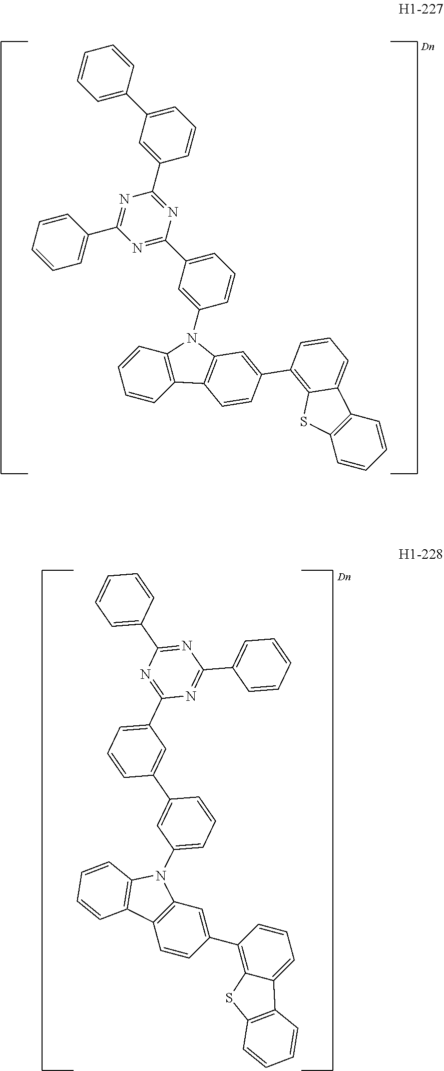

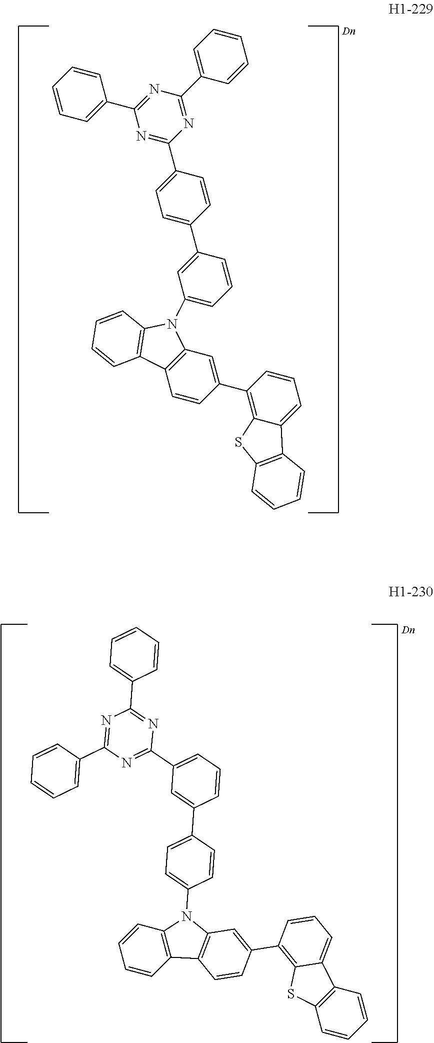

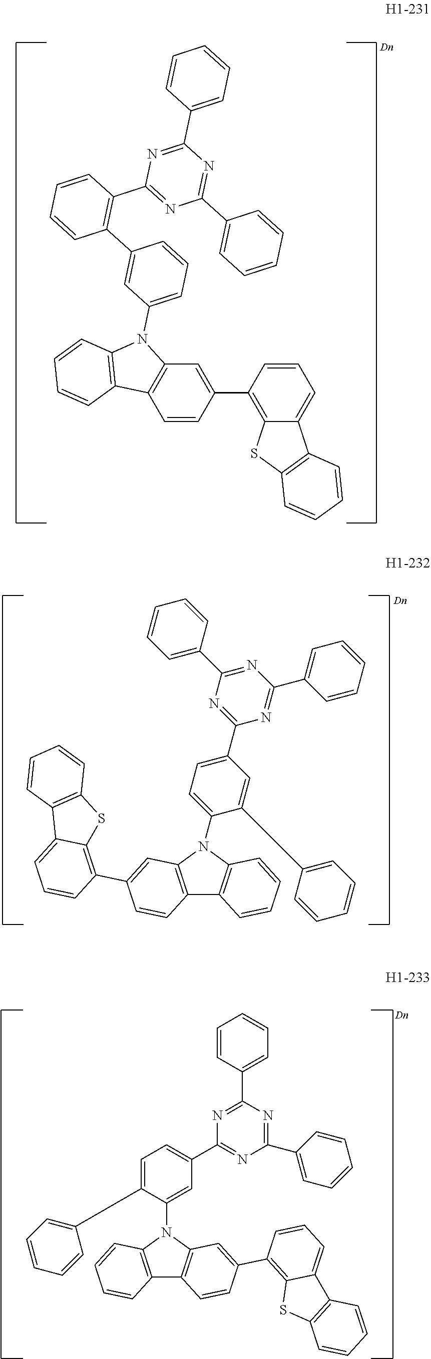

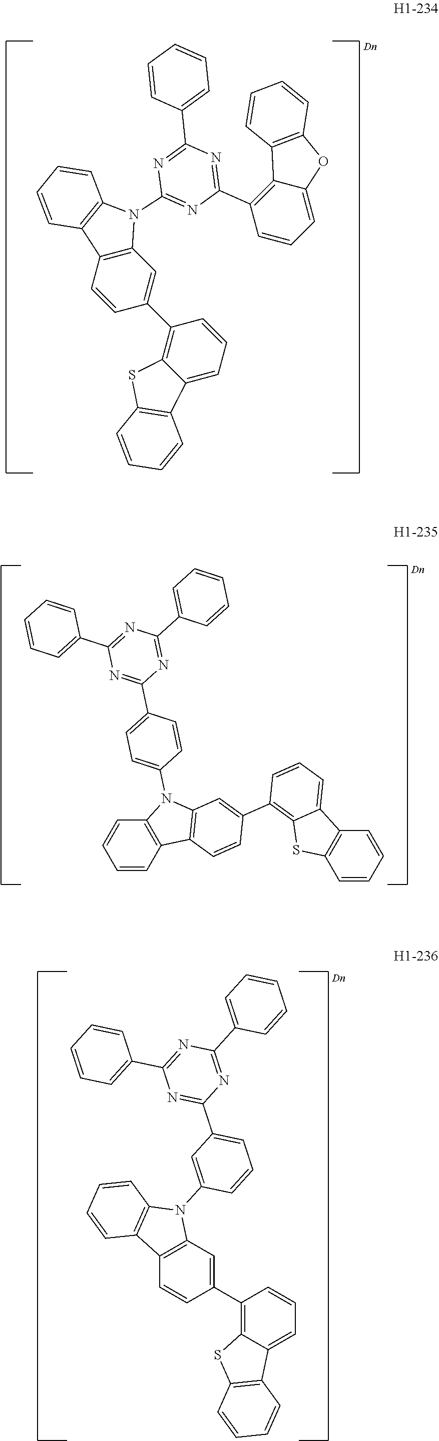

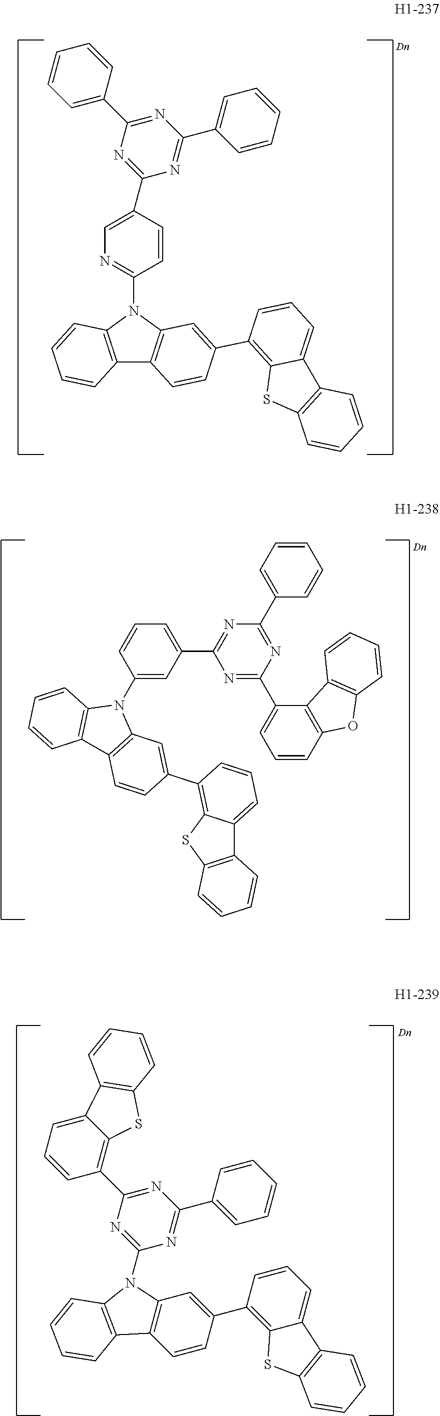

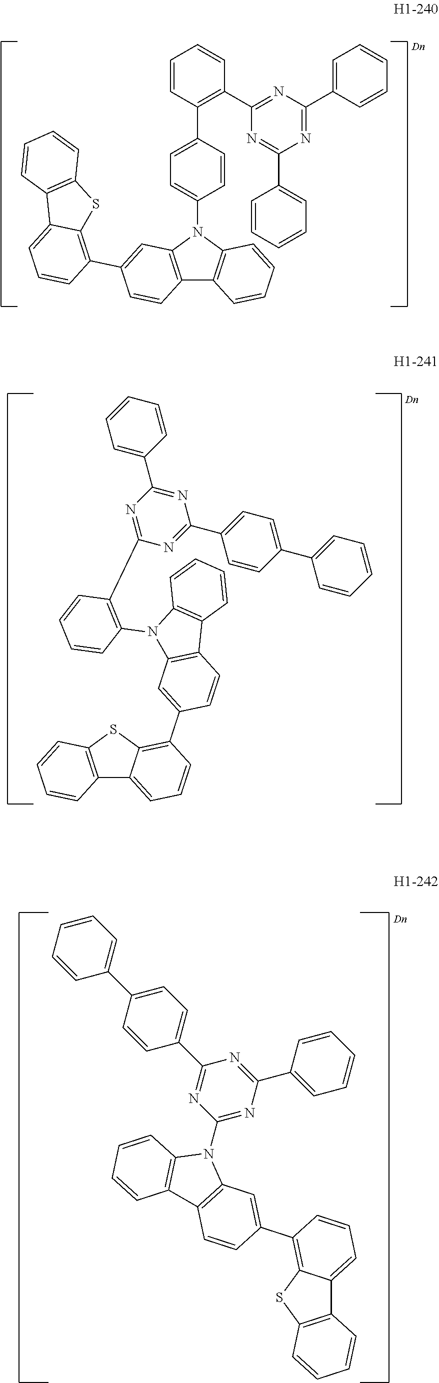

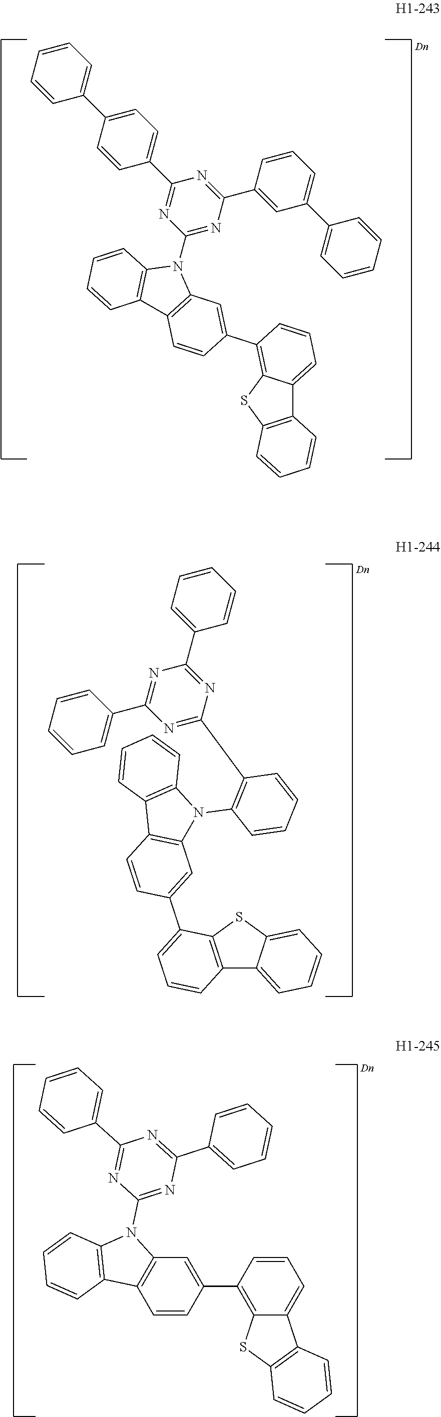

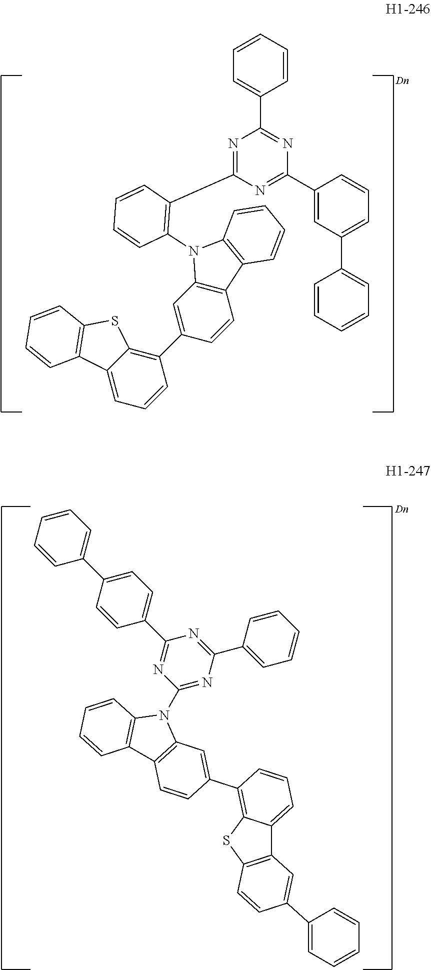

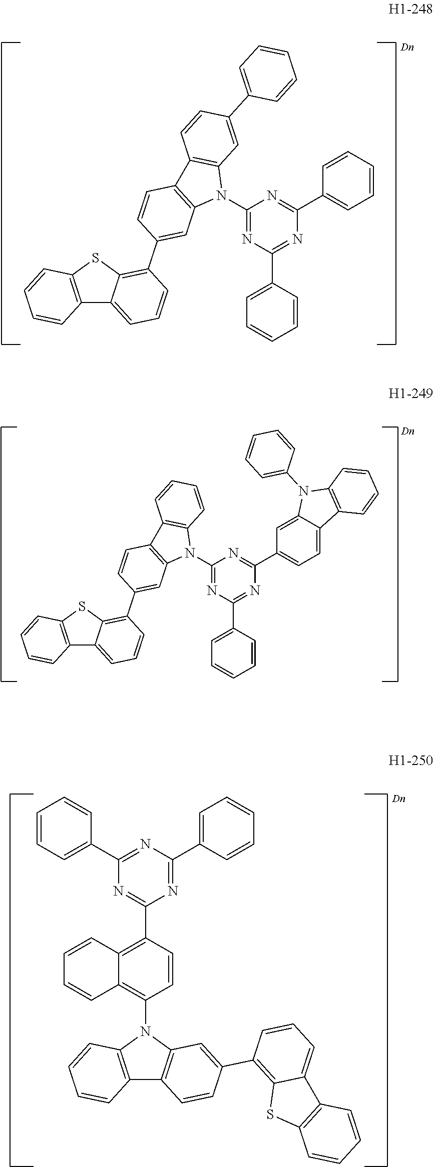

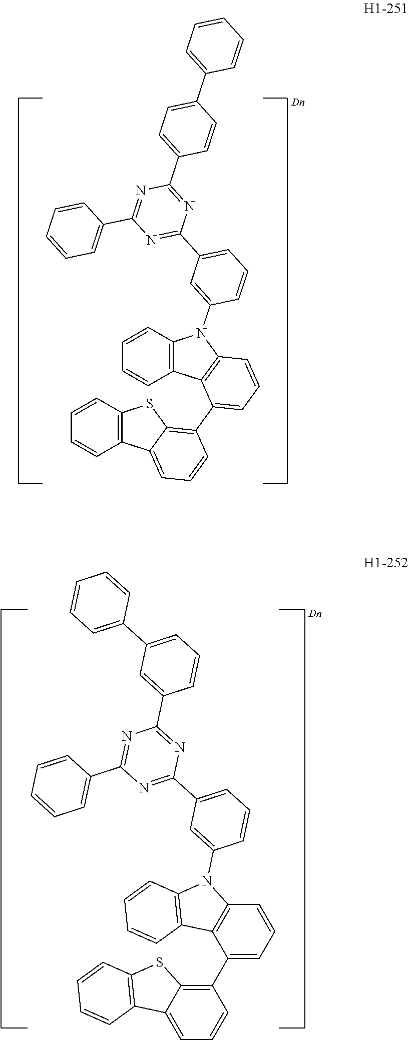

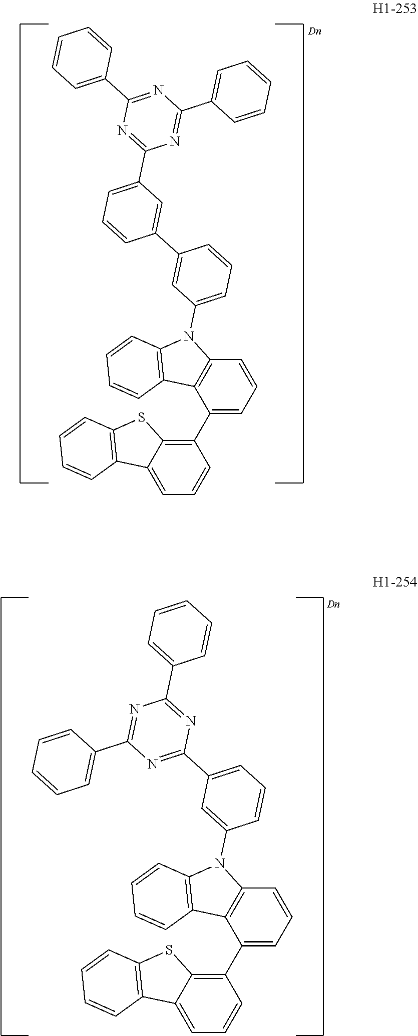

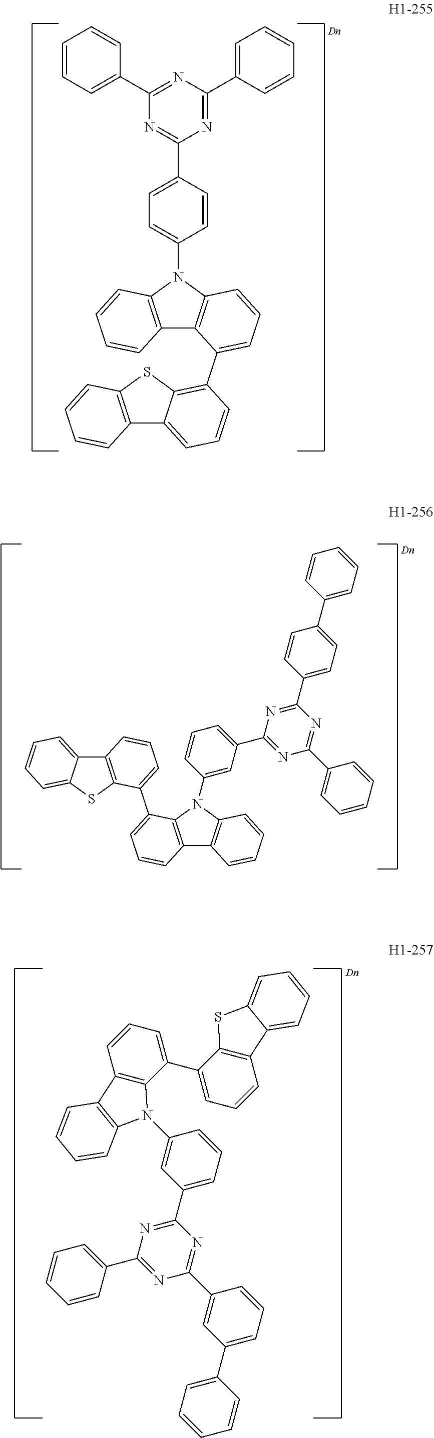

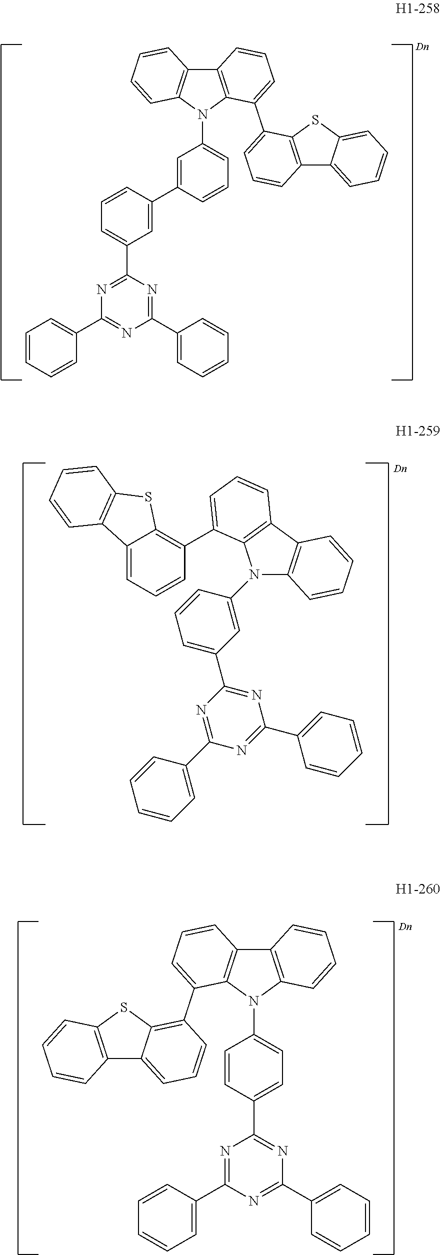

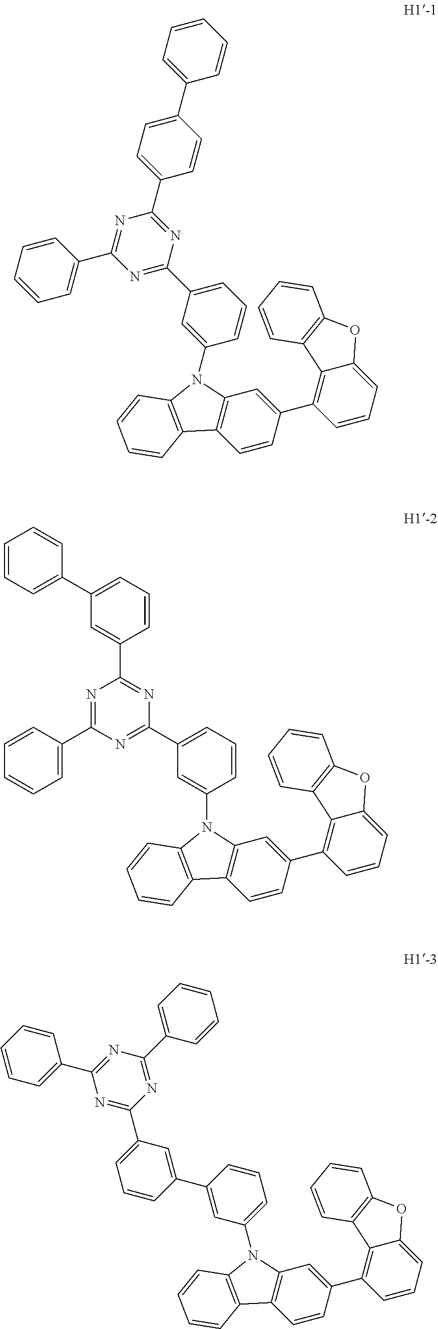 [SMILES: c1ccc(-c2ccc(-c3nc(-c4ccccc4)nc(-c4cccc(-n5c6ccccc6c6ccc(-c7cccc8oc9ccccc9c78)cc65)c4)n3)cc2)cc1 ; c1ccc(-c2cccc(-c3nc(-c4ccccc4)nc(-c4cccc(-n5c6ccccc6c6ccc(-c7cccc8oc9ccccc9c78)cc65)c4)n3)c2)cc1 ; c1ccc(-c2nc(-c3ccccc3)nc(-c3cccc(-c4cccc(-n5c6ccccc6c6ccc(-c7cccc8oc9ccccc9c78)cc65)c4)c3)n2)cc1]

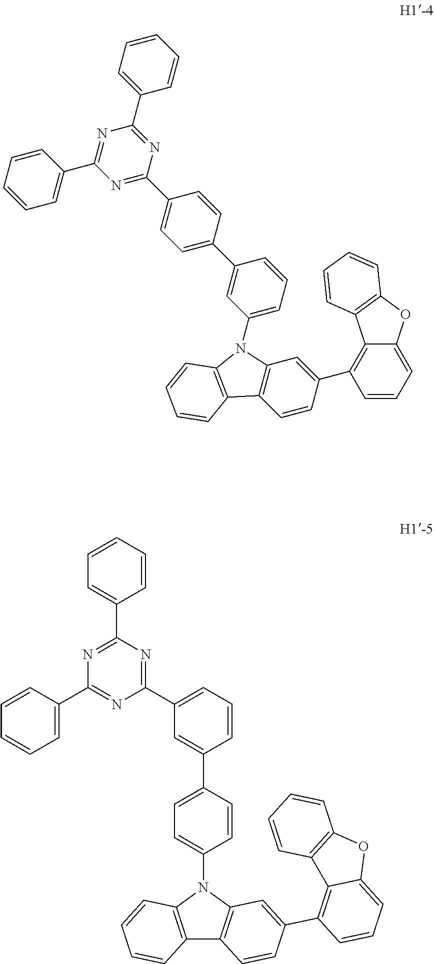 [SMILES: c1ccc(-c2nc(-c3ccccc3)nc(-c3ccc(-c4cccc(-n5c6ccccc6c6ccc(-c7cccc8oc9ccccc9c78)cc65)c4)cc3)n2)cc1 ; c1ccc(-c2nc(-c3ccccc3)nc(-c3cccc(-c4ccc(-n5c6ccccc6c6ccc(-c7cccc8oc9ccccc9c78)cc65)cc4)c3)n2)cc1]

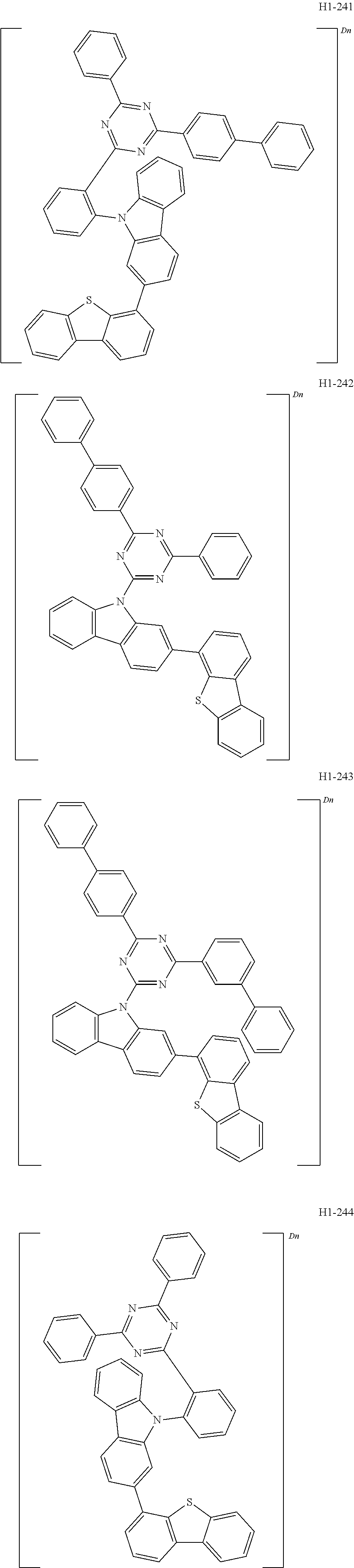

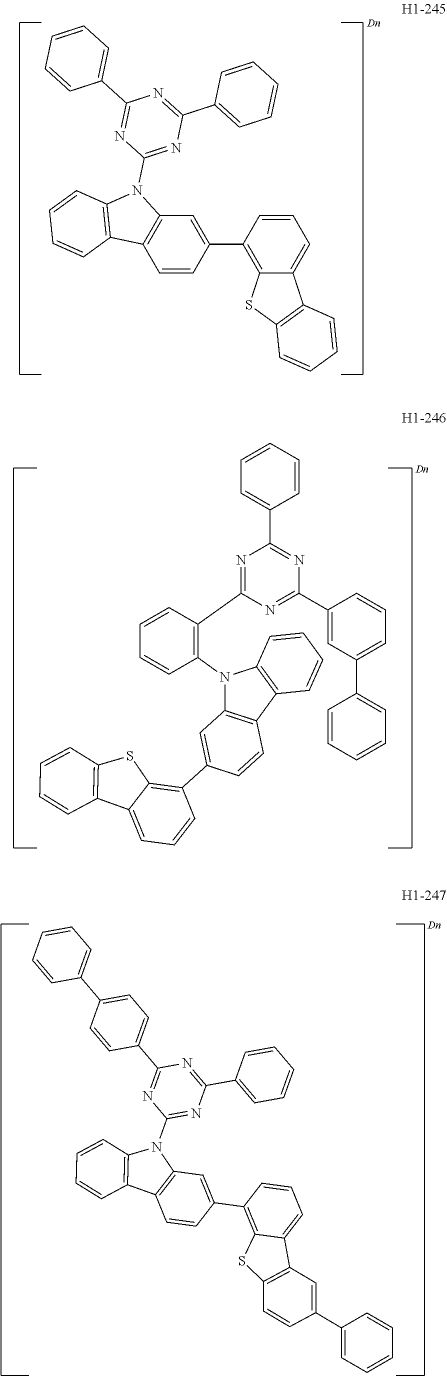

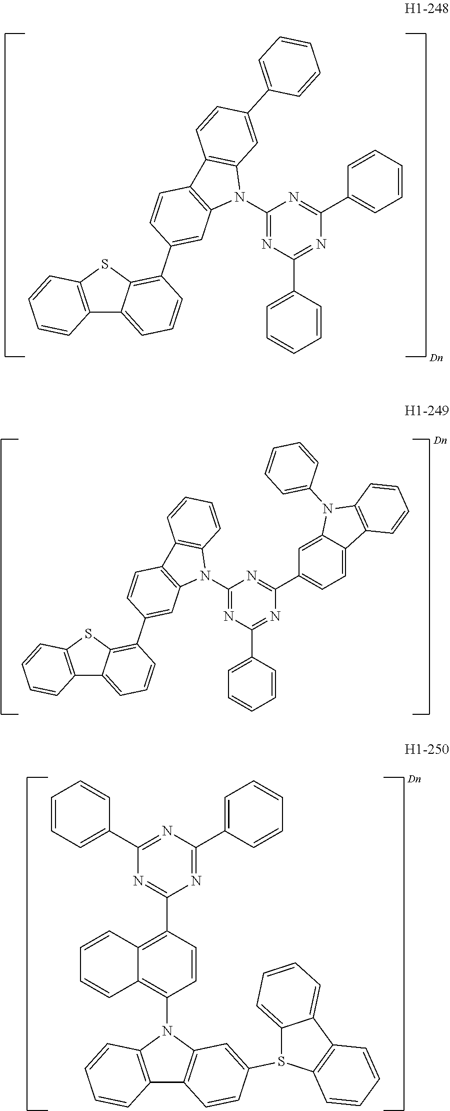

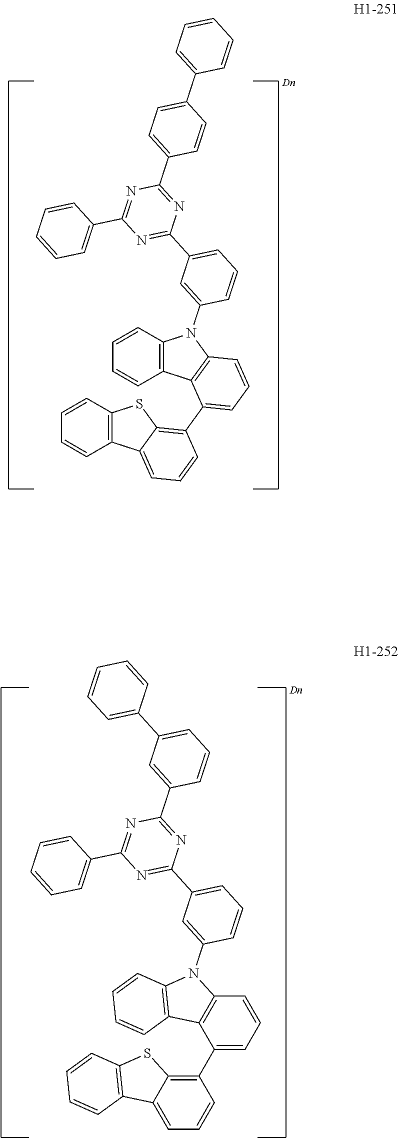

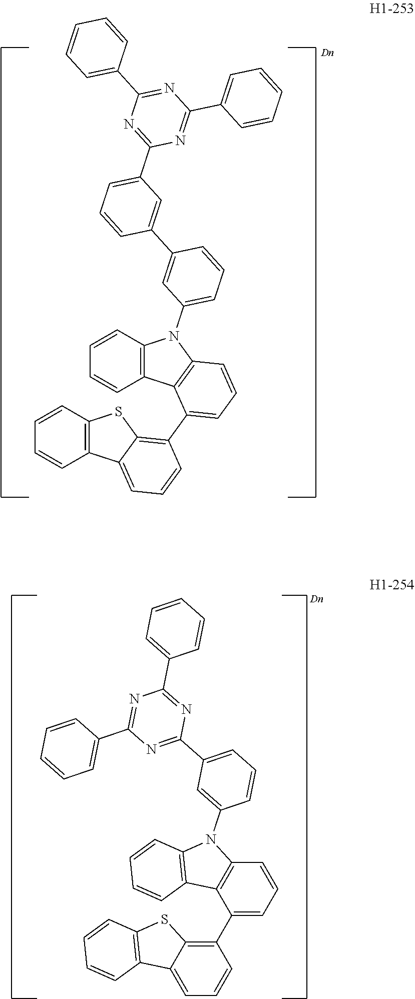

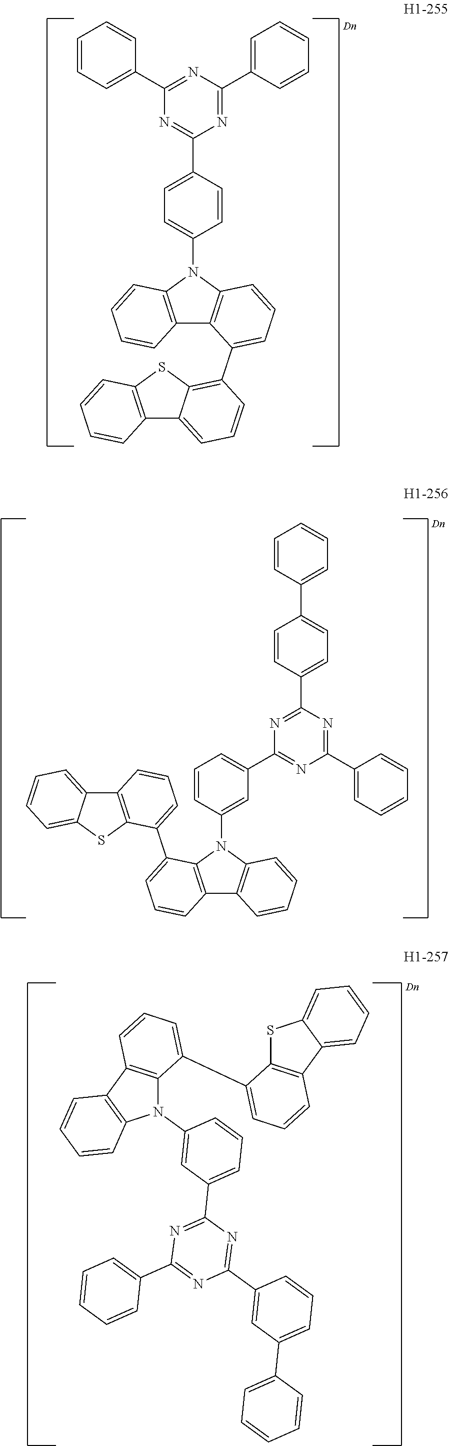

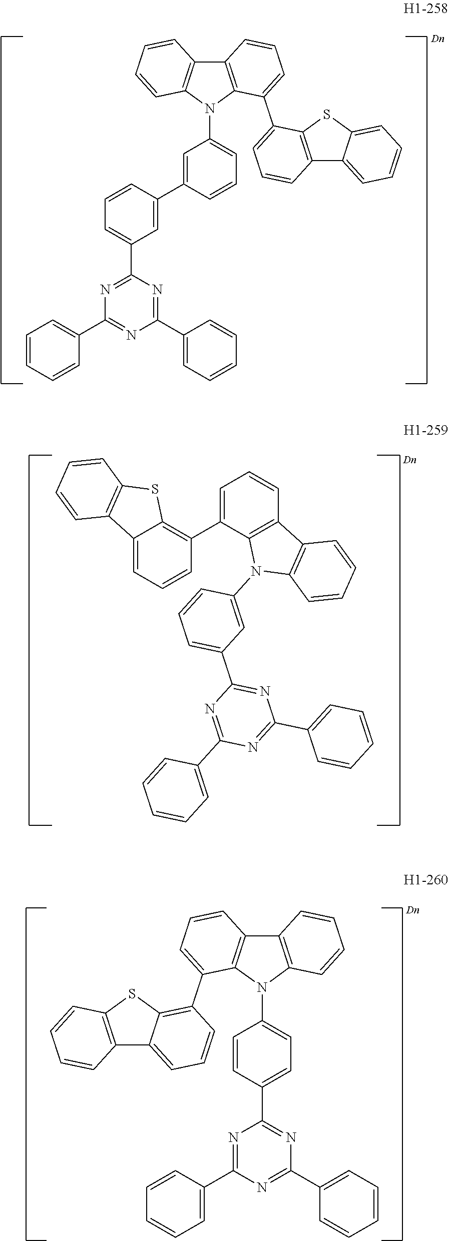

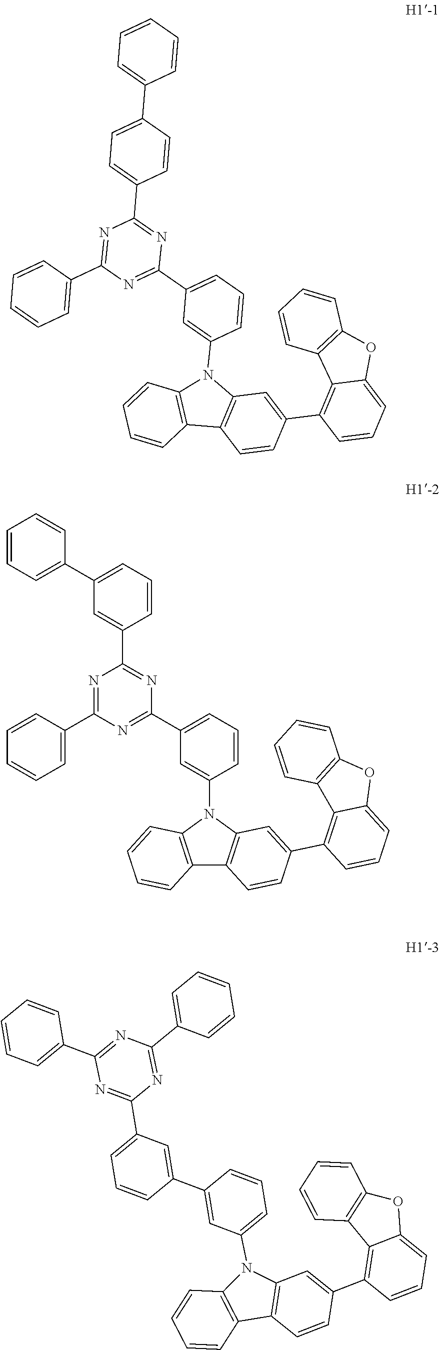 [SMILES: c1ccc(-c2ccc(-c3nc(-c4ccccc4)nc(-c4cccc(-n5c6ccccc6c6ccc(-c7cccc8oc9ccccc9c78)cc65)c4)n3)cc2)cc1 ; c1ccc(-c2cccc(-c3nc(-c4ccccc4)nc(-c4cccc(-n5c6ccccc6c6ccc(-c7cccc8oc9ccccc9c78)cc65)c4)n3)c2)cc1 ; c1ccc(-c2nc(-c3ccccc3)nc(-c3cccc(-c4cccc(-n5c6ccccc6c6ccc(-c7cccc8oc9ccccc9c78)cc65)c4)c3)n2)cc1]

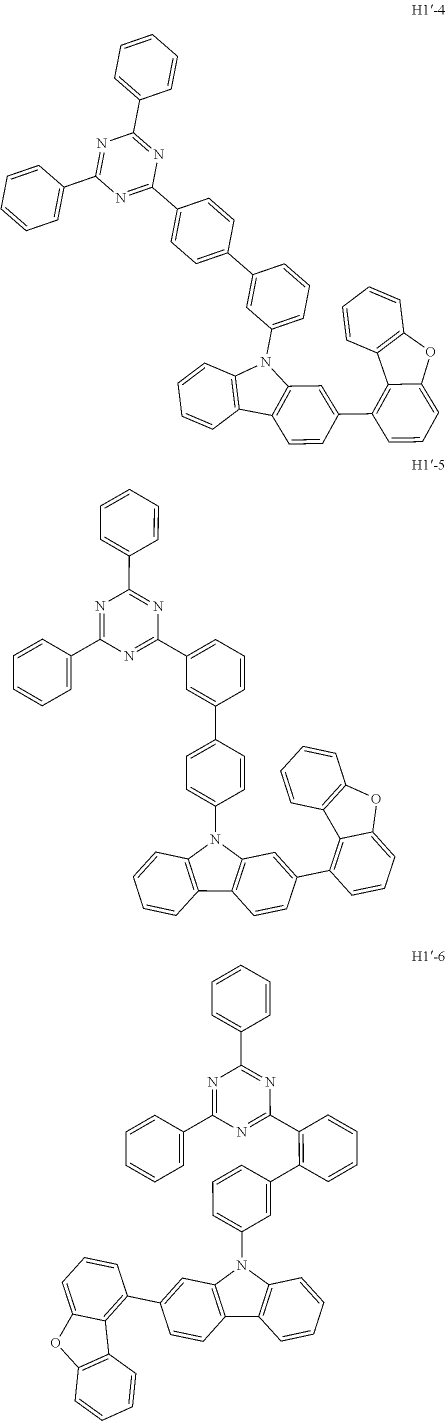 [SMILES: c1ccc(-c2nc(-c3ccccc3)nc(-c3ccc(-c4cccc(-n5c6ccccc6c6ccc(-c7cccc8oc9ccccc9c78)cc65)c4)cc3)n2)cc1 ; c1ccc(-c2nc(-c3ccccc3)nc(-c3cccc(-c4ccc(-n5c6ccccc6c6ccc(-c7cccc8oc9ccccc9c78)cc65)cc4)c3)n2)cc1 ; c1ccc(-c2nc(-c3ccccc3)nc(-c3ccccc3-c3cccc(-n4c5ccccc5c5ccc(-c6cccc7oc8ccccc8c67)cc54)c3)n2)cc1]

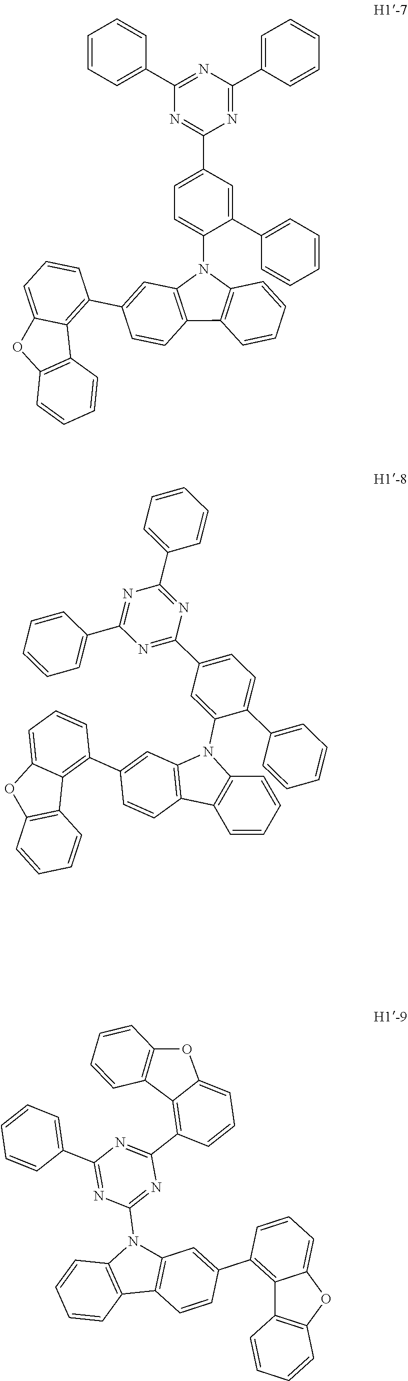 [SMILES: c1ccc(-c2nc(-c3ccccc3)nc(-c3ccc(-n4c5ccccc5c5ccc(-c6cccc7oc8ccccc8c67)cc54)c(-c4ccccc4)c3)n2)cc1 ; c1ccc(-c2nc(-c3ccccc3)nc(-c3ccc(-c4ccccc4)c(-n4c5ccccc5c5ccc(-c6cccc7oc8ccccc8c67)cc54)c3)n2)cc1 ; c1ccc(-c2nc(-c3cccc4oc5ccccc5c34)nc(-n3c4ccccc4c4ccc(-c5cccc6oc7ccccc7c56)cc43)n2)cc1]

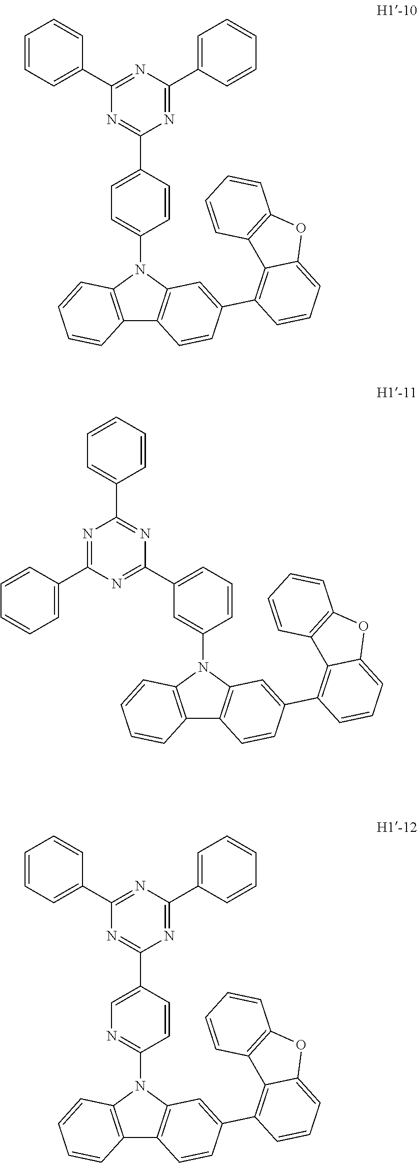 [SMILES: c1ccc(-c2nc(-c3ccccc3)nc(-c3ccc(-n4c5ccccc5c5ccc(-c6cccc7oc8ccccc8c67)cc54)cc3)n2)cc1 ; c1ccc(-c2nc(-c3ccccc3)nc(-c3cccc(-n4c5ccccc5c5ccc(-c6cccc7oc8ccccc8c67)cc54)c3)n2)cc1 ; c1ccc(-c2nc(-c3ccccc3)nc(-c3ccc(-n4c5ccccc5c5ccc(-c6cccc7oc8ccccc8c67)cc54)nc3)n2)cc1]

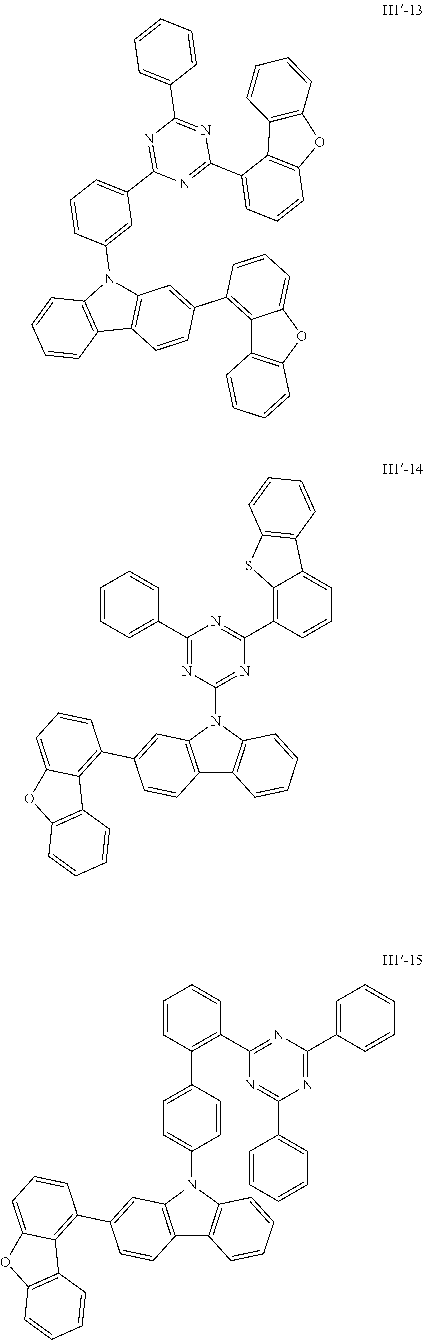 [SMILES: c1ccc(-c2nc(-c3cccc(-n4c5ccccc5c5ccc(-c6cccc7oc8ccccc8c67)cc54)c3)nc(-c3cccc4oc5ccccc5c34)n2)cc1 ; c1ccc(-c2nc(-c3cccc4c3sc3ccccc34)nc(-n3c4ccccc4c4ccc(-c5cccc6oc7ccccc7c56)cc43)n2)cc1 ; c1ccc(-c2nc(-c3ccccc3)nc(-c3ccccc3-c3ccc(-n4c5ccccc5c5ccc(-c6cccc7oc8ccccc8c67)cc54)cc3)n2)cc1]

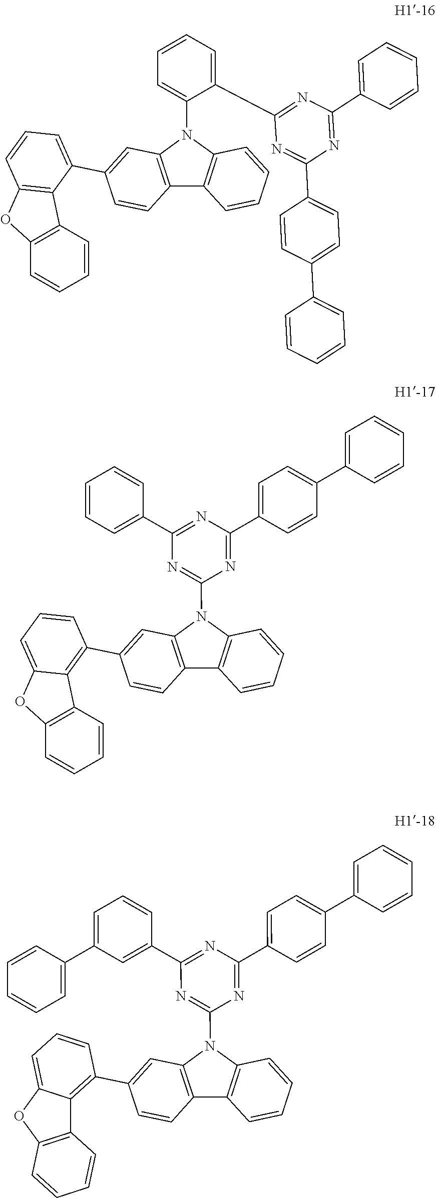 [SMILES: c1ccc(-c2ccc(-c3nc(-c4ccccc4)nc(-c4ccccc4-n4c5ccccc5c5ccc(-c6cccc7oc8ccccc8c67)cc54)n3)cc2)cc1 ; c1ccc(-c2ccc(-c3nc(-c4ccccc4)nc(-n4c5ccccc5c5ccc(-c6cccc7oc8ccccc8c67)cc54)n3)cc2)cc1 ; c1ccc(-c2ccc(-c3nc(-c4cccc(-c5ccccc5)c4)nc(-n4c5ccccc5c5ccc(-c6cccc7oc8ccccc8c67)cc54)n3)cc2)cc1]

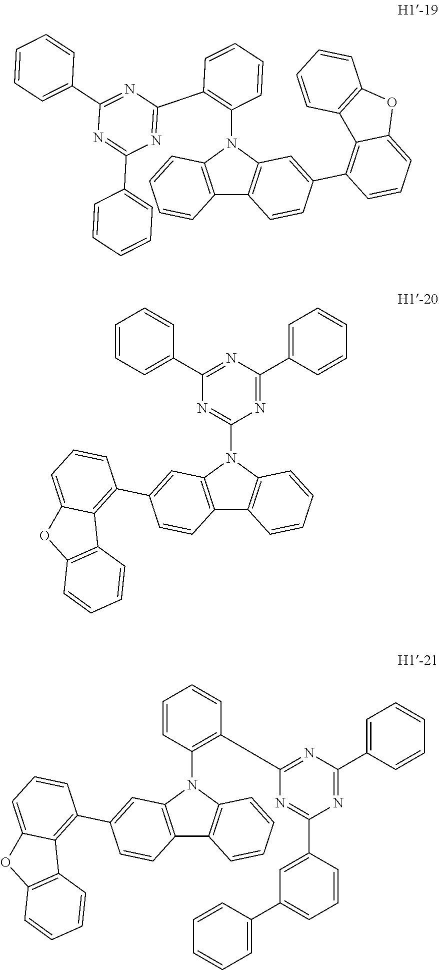 [SMILES: c1ccc(-c2nc(-c3ccccc3)nc(-c3ccccc3-n3c4ccccc4c4ccc(-c5cccc6oc7ccccc7c56)cc43)n2)cc1 ; c1ccc(-c2nc(-c3ccccc3)nc(-n3c4ccccc4c4ccc(-c5cccc6oc7ccccc7c56)cc43)n2)cc1 ; c1ccc(-c2cccc(-c3nc(-c4ccccc4)nc(-c4ccccc4-n4c5ccccc5c5ccc(-c6cccc7oc8ccccc8c67)cc54)n3)c2)cc1]

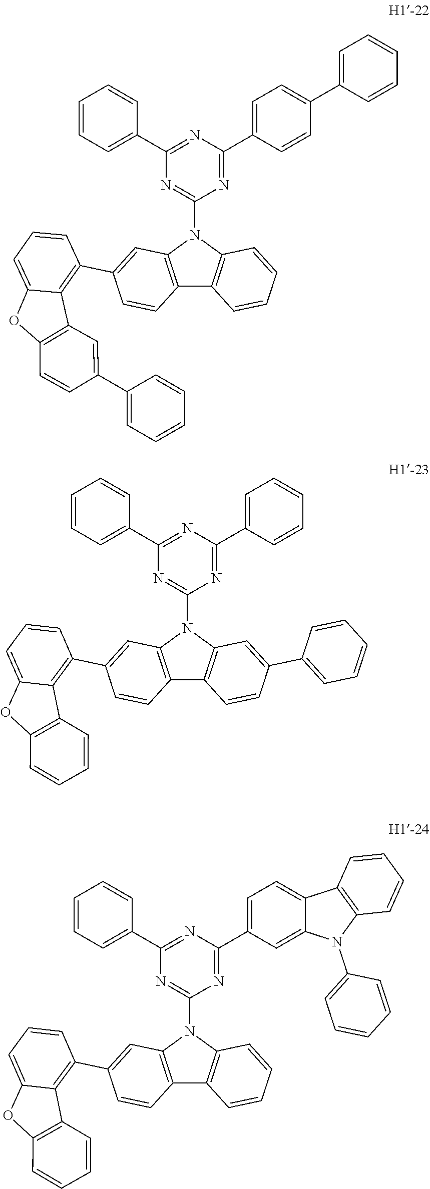 [SMILES: c1ccc(-c2ccc(-c3nc(-c4ccccc4)nc(-n4c5ccccc5c5ccc(-c6cccc7oc8ccc(-c9ccccc9)cc8c67)cc54)n3)cc2)cc1 ; c1ccc(-c2ccc3c4ccc(-c5cccc6oc7ccccc7c56)cc4n(-c4nc(-c5ccccc5)nc(-c5ccccc5)n4)c3c2)cc1 ; c1ccc(-c2nc(-c3ccc4c5ccccc5n(-c5ccccc5)c4c3)nc(-n3c4ccccc4c4ccc(-c5cccc6oc7ccccc7c56)cc43)n2)cc1]

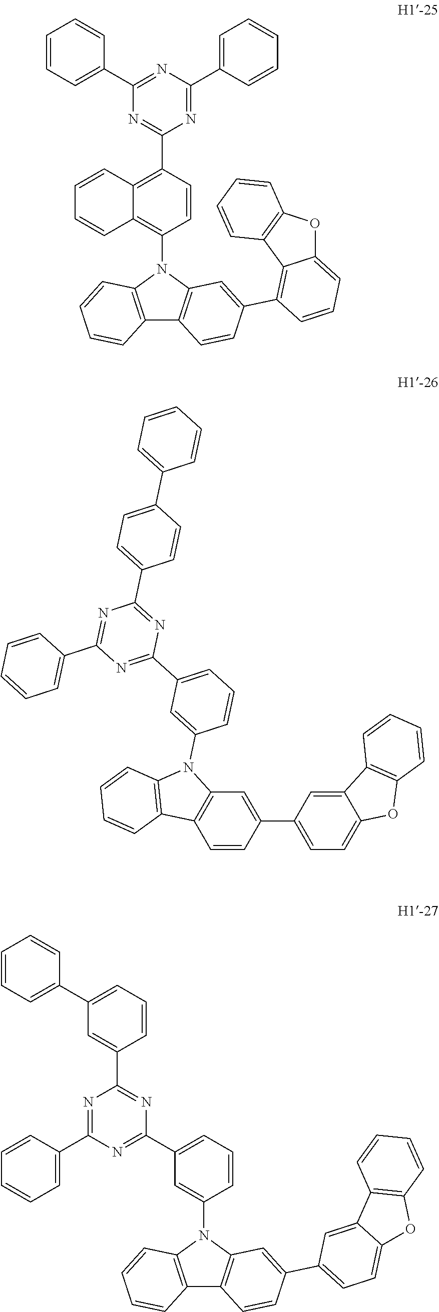 [SMILES: c1ccc(-c2nc(-c3ccccc3)nc(-c3ccc(-n4c5ccccc5c5ccc(-c6cccc7oc8ccccc8c67)cc54)c4ccccc34)n2)cc1 ; c1ccc(-c2ccc(-c3nc(-c4ccccc4)nc(-c4cccc(-n5c6ccccc6c6ccc(-c7ccc8oc9ccccc9c8c7)cc65)c4)n3)cc2)cc1 ; c1ccc(-c2cccc(-c3nc(-c4ccccc4)nc(-c4cccc(-n5c6ccccc6c6ccc(-c7ccc8oc9ccccc9c8c7)cc65)c4)n3)c2)cc1]

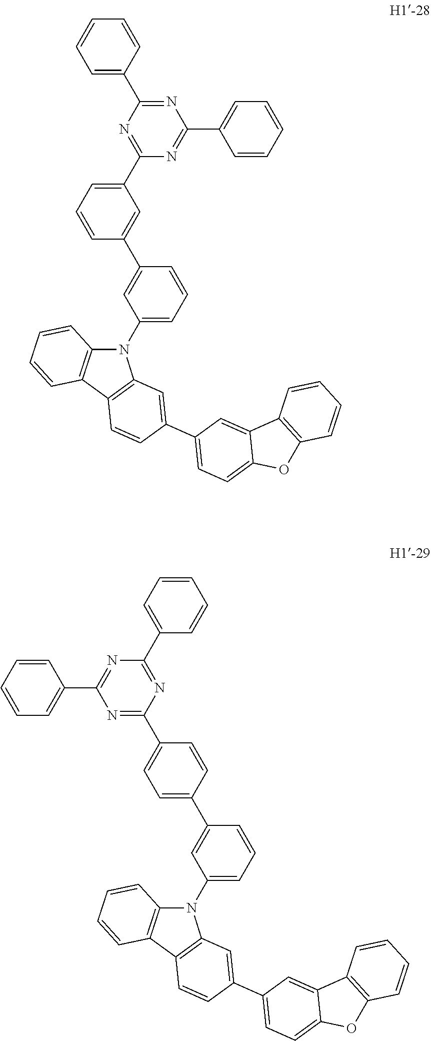 [SMILES: c1ccc(-c2nc(-c3ccccc3)nc(-c3cccc(-c4cccc(-n5c6ccccc6c6ccc(-c7ccc8oc9ccccc9c8c7)cc65)c4)c3)n2)cc1 ; c1ccc(-c2nc(-c3ccccc3)nc(-c3ccc(-c4cccc(-n5c6ccccc6c6ccc(-c7ccc8oc9ccccc9c8c7)cc65)c4)cc3)n2)cc1]

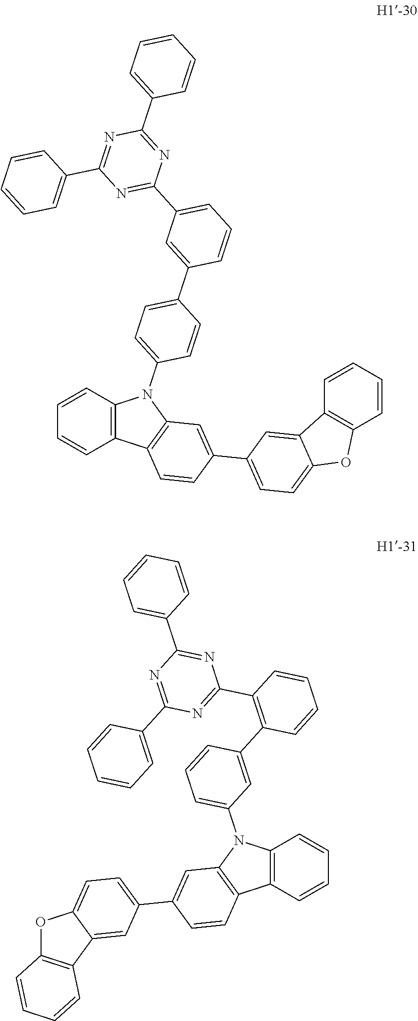 [SMILES: c1ccc(-c2nc(-c3ccccc3)nc(-c3cccc(-c4ccc(-n5c6ccccc6c6ccc(-c7ccc8oc9ccccc9c8c7)cc65)cc4)c3)n2)cc1 ; c1ccc(-c2nc(-c3ccccc3)nc(-c3ccccc3-c3cccc(-n4c5ccccc5c5ccc(-c6ccc7oc8ccccc8c7c6)cc54)c3)n2)cc1]

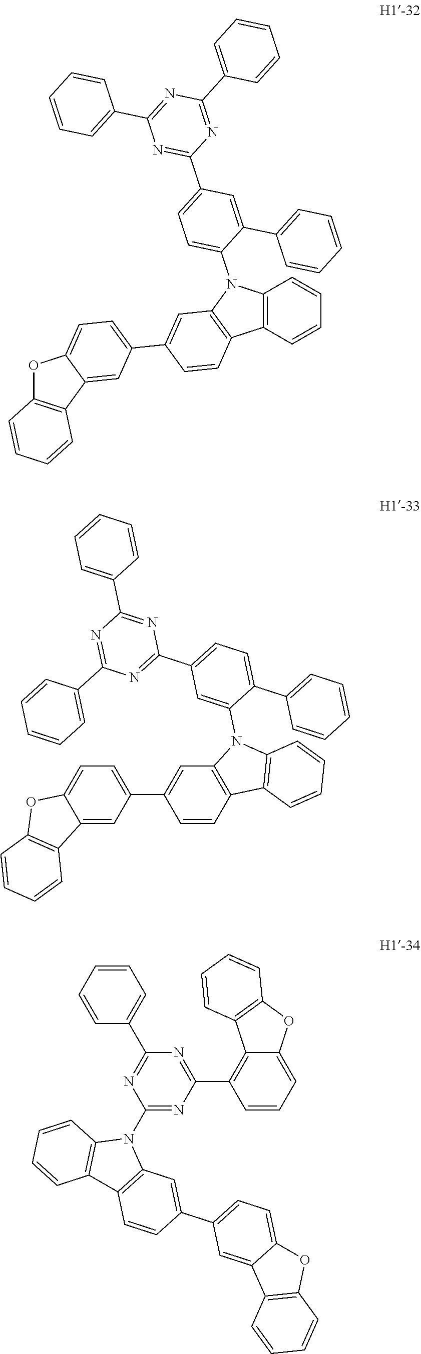 [SMILES: c1ccc(-c2nc(-c3ccccc3)nc(-c3ccc(-n4c5ccccc5c5ccc(-c6ccc7oc8ccccc8c7c6)cc54)c(-c4ccccc4)c3)n2)cc1 ; c1ccc(-c2nc(-c3ccccc3)nc(-c3ccc(-c4ccccc4)c(-n4c5ccccc5c5ccc(-c6ccc7oc8ccccc8c7c6)cc54)c3)n2)cc1 ; c1ccc(-c2nc(-c3cccc4oc5ccccc5c34)nc(-n3c4ccccc4c4ccc(-c5ccc6oc7ccccc7c6c5)cc43)n2)cc1]

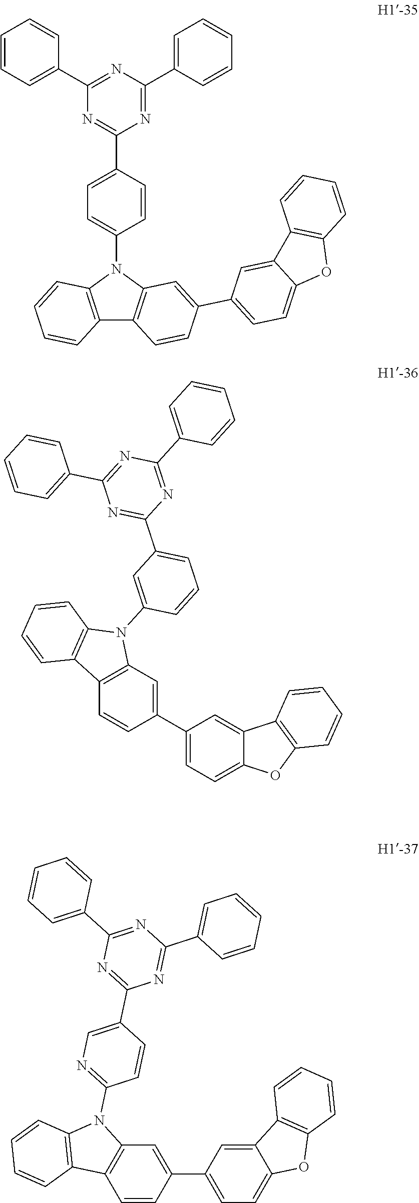

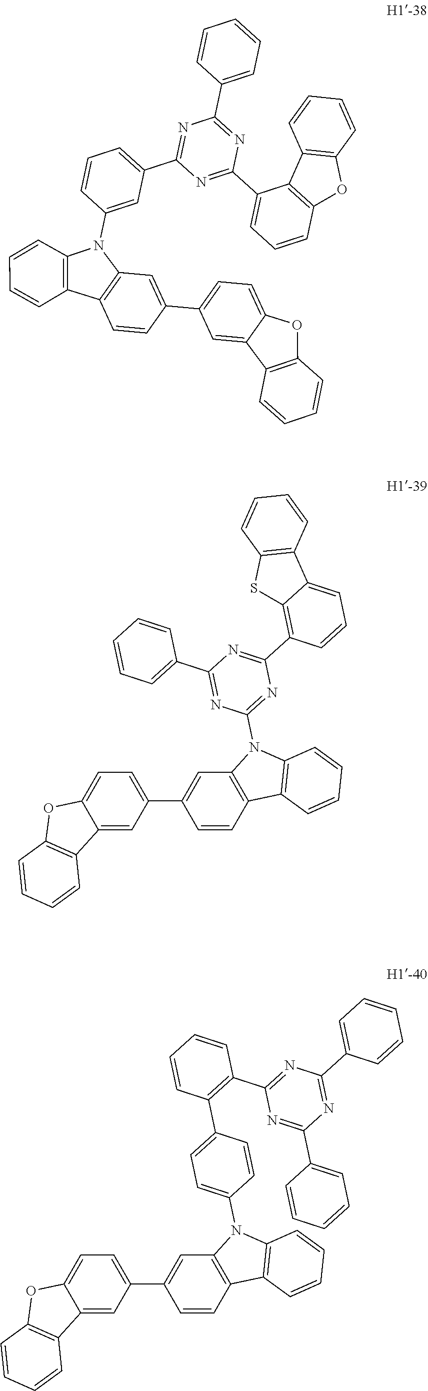 [SMILES: c1ccc(-c2nc(-c3cccc(-n4c5ccccc5c5ccc(-c6ccc7oc8ccccc8c7c6)cc54)c3)nc(-c3cccc4oc5ccccc5c34)n2)cc1 ; c1ccc(-c2nc(-c3cccc4c3sc3ccccc34)nc(-n3c4ccccc4c4ccc(-c5ccc6oc7ccccc7c6c5)cc43)n2)cc1 ; c1ccc(-c2nc(-c3ccccc3)nc(-c3ccccc3-c3ccc(-n4c5ccccc5c5ccc(-c6ccc7oc8ccccc8c7c6)cc54)cc3)n2)cc1]

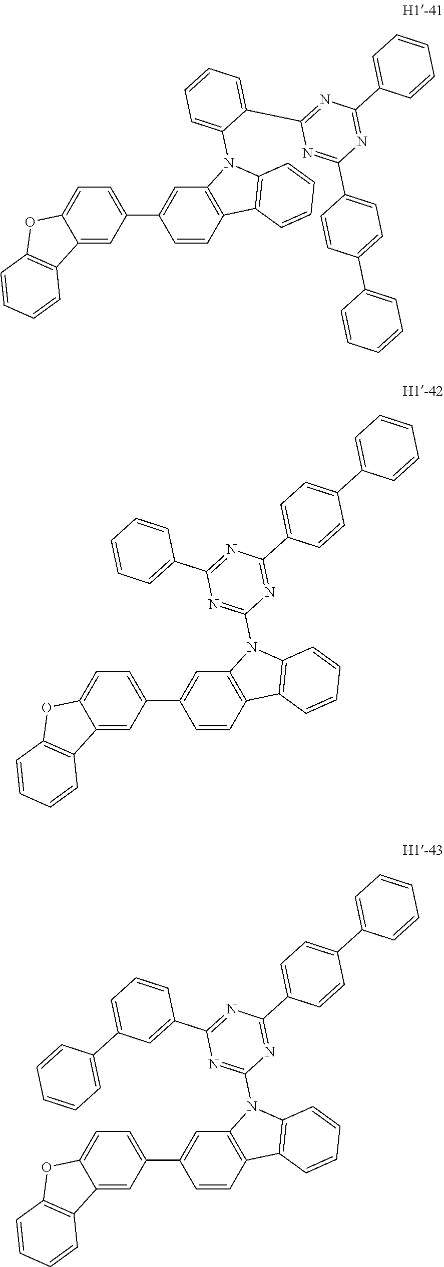 [SMILES: c1ccc(-c2ccc(-c3nc(-c4ccccc4)nc(-c4ccccc4-n4c5ccccc5c5ccc(-c6ccc7oc8ccccc8c7c6)cc54)n3)cc2)cc1 ; c1ccc(-c2ccc(-c3nc(-c4ccccc4)nc(-n4c5ccccc5c5ccc(-c6ccc7oc8ccccc8c7c6)cc54)n3)cc2)cc1 ; c1ccc(-c2ccc(-c3nc(-c4cccc(-c5ccccc5)c4)nc(-n4c5ccccc5c5ccc(-c6ccc7oc8ccccc8c7c6)cc54)n3)cc2)cc1]

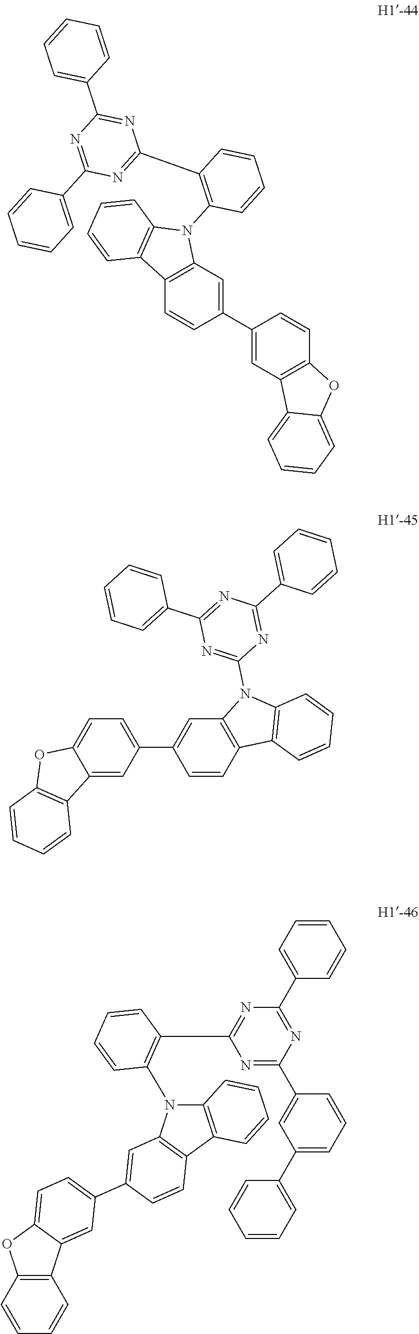 [SMILES: c1ccc(-c2nc(-c3ccccc3)nc(-c3ccccc3-n3c4ccccc4c4ccc(-c5ccc6oc7ccccc7c6c5)cc43)n2)cc1 ; c1ccc(-c2nc(-c3ccccc3)nc(-n3c4ccccc4c4ccc(-c5ccc6oc7ccccc7c6c5)cc43)n2)cc1 ; c1ccc(-c2cccc(-c3nc(-c4ccccc4)nc(-c4ccccc4-n4c5ccccc5c5ccc(-c6ccc7oc8ccccc8c7c6)cc54)n3)c2)cc1]

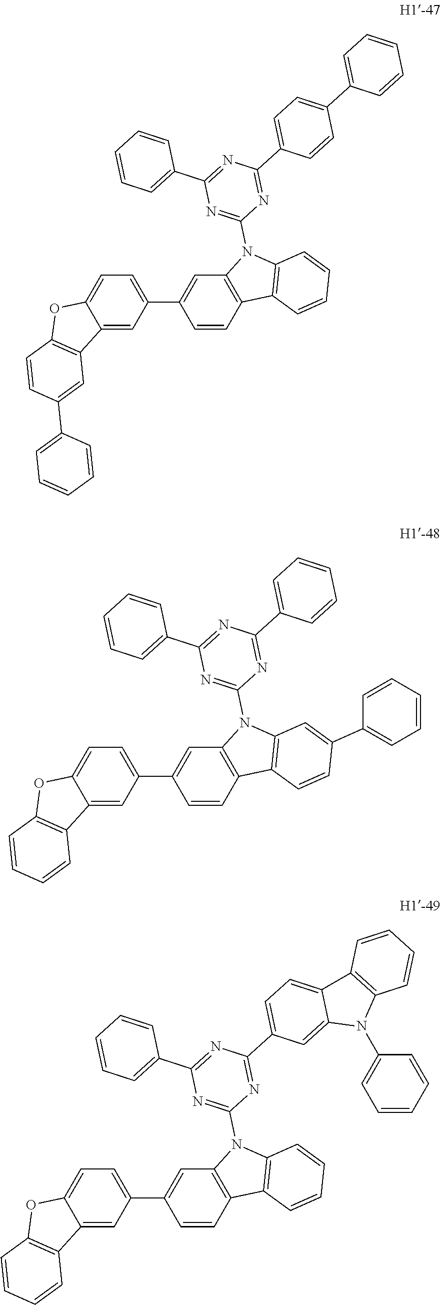 [SMILES: c1ccc(-c2ccc(-c3nc(-c4ccccc4)nc(-n4c5ccccc5c5ccc(-c6ccc7oc8ccc(-c9ccccc9)cc8c7c6)cc54)n3)cc2)cc1 ; c1ccc(-c2ccc3c4ccc(-c5ccc6oc7ccccc7c6c5)cc4n(-c4nc(-c5ccccc5)nc(-c5ccccc5)n4)c3c2)cc1 ; c1ccc(-c2nc(-c3ccc4c5ccccc5n(-c5ccccc5)c4c3)nc(-n3c4ccccc4c4ccc(-c5ccc6oc7ccccc7c6c5)cc43)n2)cc1]

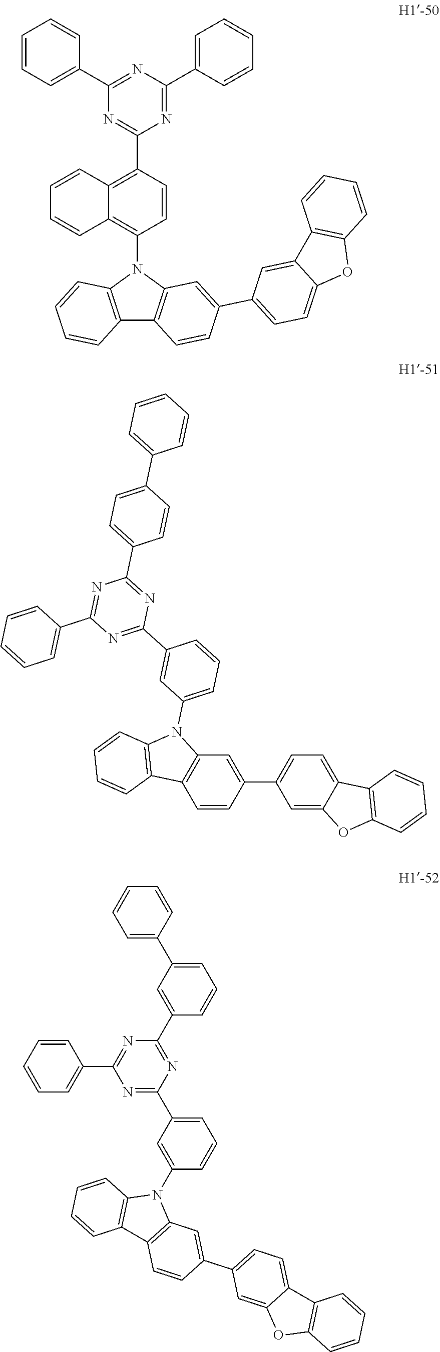 [SMILES: c1ccc(-c2nc(-c3ccccc3)nc(-c3ccc(-n4c5ccccc5c5ccc(-c6ccc7oc8ccccc8c7c6)cc54)c4ccccc34)n2)cc1 ; c1ccc(-c2ccc(-c3nc(-c4ccccc4)nc(-c4cccc(-n5c6ccccc6c6ccc(-c7ccc8c(c7)oc7ccccc78)cc65)c4)n3)cc2)cc1 ; c1ccc(-c2cccc(-c3nc(-c4ccccc4)nc(-c4cccc(-n5c6ccccc6c6ccc(-c7ccc8c(c7)oc7ccccc78)cc65)c4)n3)c2)cc1]

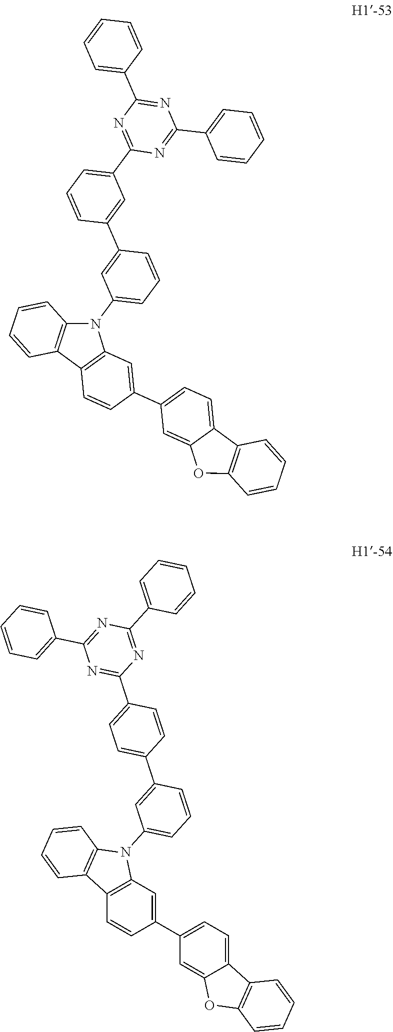 [SMILES: c1ccc(-c2nc(-c3ccccc3)nc(-c3cccc(-c4cccc(-n5c6ccccc6c6ccc(-c7ccc8c(c7)oc7ccccc78)cc65)c4)c3)n2)cc1 ; c1ccc(-c2nc(-c3ccccc3)nc(-c3ccc(-c4cccc(-n5c6ccccc6c6ccc(-c7ccc8c(c7)oc7ccccc78)cc65)c4)cc3)n2)cc1]

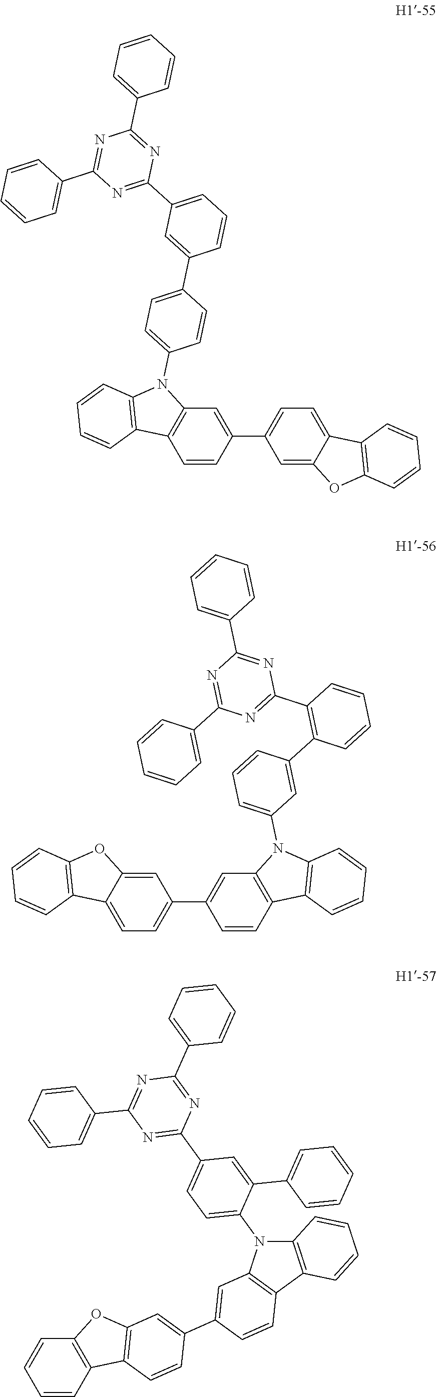 [SMILES: c1ccc(-c2nc(-c3ccccc3)nc(-c3cccc(-c4ccc(-n5c6ccccc6c6ccc(-c7ccc8c(c7)oc7ccccc78)cc65)cc4)c3)n2)cc1 ; c1ccc(-c2nc(-c3ccccc3)nc(-c3ccccc3-c3cccc(-n4c5ccccc5c5ccc(-c6ccc7c(c6)oc6ccccc67)cc54)c3)n2)cc1 ; c1ccc(-c2nc(-c3ccccc3)nc(-c3ccc(-n4c5ccccc5c5ccc(-c6ccc7c(c6)oc6ccccc67)cc54)c(-c4ccccc4)c3)n2)cc1]

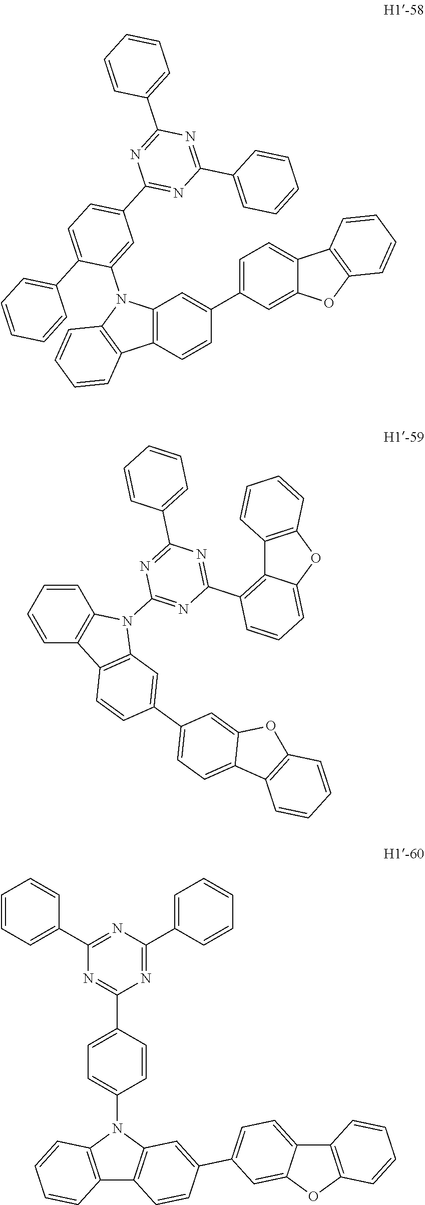 [SMILES: c1ccc(-c2nc(-c3ccccc3)nc(-c3ccc(-c4ccccc4)c(-n4c5ccccc5c5ccc(-c6ccc7c(c6)oc6ccccc67)cc54)c3)n2)cc1 ; c1ccc(-c2nc(-c3cccc4oc5ccccc5c34)nc(-n3c4ccccc4c4ccc(-c5ccc6c(c5)oc5ccccc56)cc43)n2)cc1 ; c1ccc(-c2nc(-c3ccccc3)nc(-c3ccc(-n4c5ccccc5c5ccc(-c6ccc7c(c6)oc6ccccc67)cc54)cc3)n2)cc1]

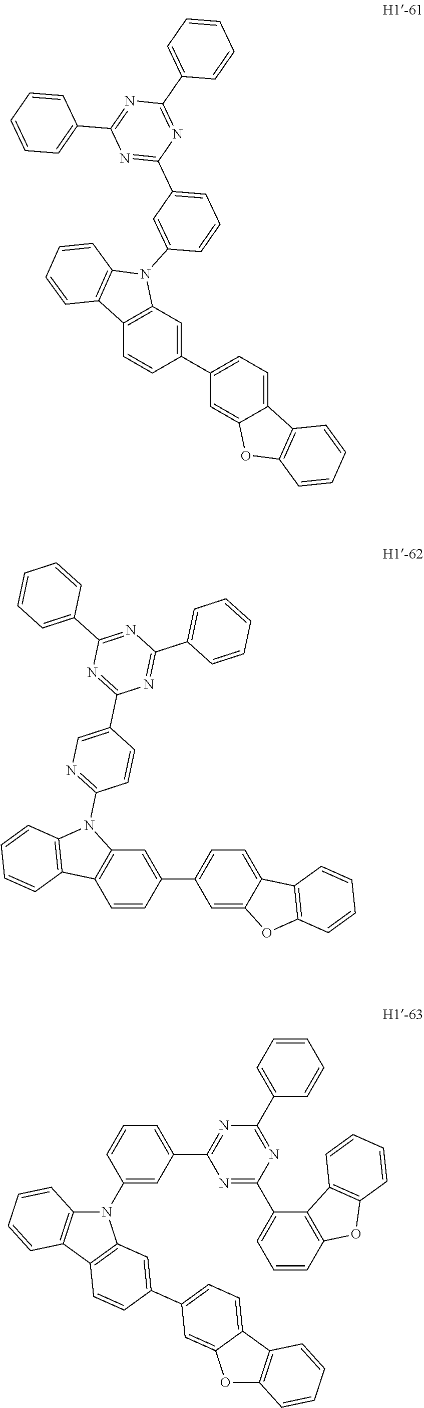 [SMILES: c1ccc(-c2nc(-c3ccccc3)nc(-c3cccc(-n4c5ccccc5c5ccc(-c6ccc7c(c6)oc6ccccc67)cc54)c3)n2)cc1 ; c1ccc(-c2nc(-c3ccccc3)nc(-c3ccc(-n4c5ccccc5c5ccc(-c6ccc7c(c6)oc6ccccc67)cc54)nc3)n2)cc1 ; c1ccc(-c2nc(-c3cccc(-n4c5ccccc5c5ccc(-c6ccc7c(c6)oc6ccccc67)cc54)c3)nc(-c3cccc4oc5ccccc5c34)n2)cc1]

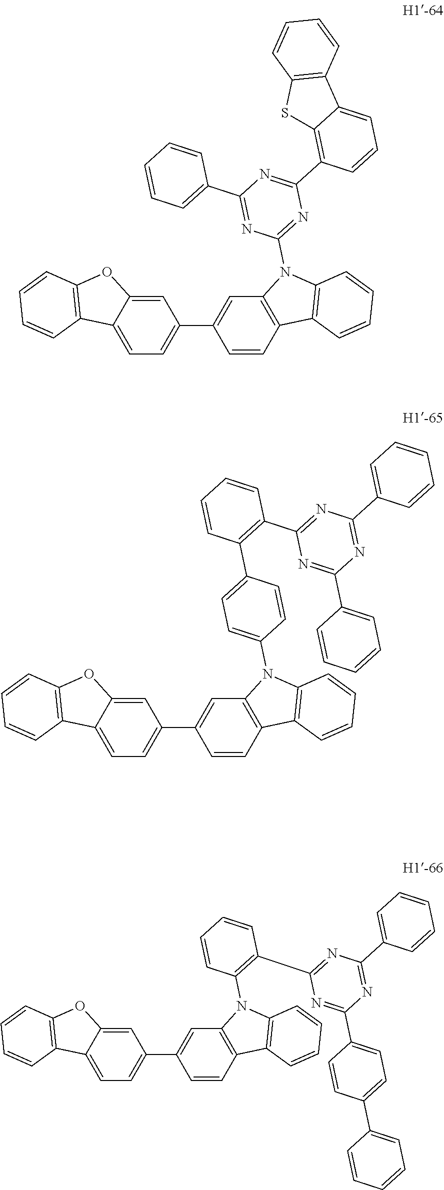 [SMILES: c1ccc(-c2nc(-c3cccc4c3sc3ccccc34)nc(-n3c4ccccc4c4ccc(-c5ccc6c(c5)oc5ccccc56)cc43)n2)cc1 ; c1ccc(-c2nc(-c3ccccc3)nc(-c3ccccc3-c3ccc(-n4c5ccccc5c5ccc(-c6ccc7c(c6)oc6ccccc67)cc54)cc3)n2)cc1 ; c1ccc(-c2ccc(-c3nc(-c4ccccc4)nc(-c4ccccc4-n4c5ccccc5c5ccc(-c6ccc7c(c6)oc6ccccc67)cc54)n3)cc2)cc1]

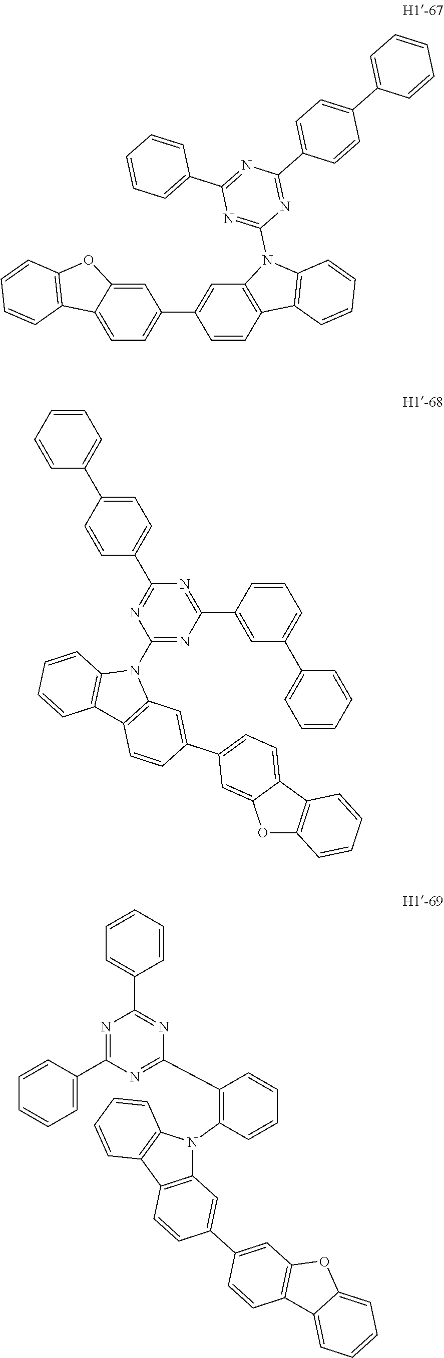 [SMILES: c1ccc(-c2ccc(-c3nc(-c4ccccc4)nc(-n4c5ccccc5c5ccc(-c6ccc7c(c6)oc6ccccc67)cc54)n3)cc2)cc1 ; c1ccc(-c2ccc(-c3nc(-c4cccc(-c5ccccc5)c4)nc(-n4c5ccccc5c5ccc(-c6ccc7c(c6)oc6ccccc67)cc54)n3)cc2)cc1 ; c1ccc(-c2nc(-c3ccccc3)nc(-c3ccccc3-n3c4ccccc4c4ccc(-c5ccc6c(c5)oc5ccccc56)cc43)n2)cc1]

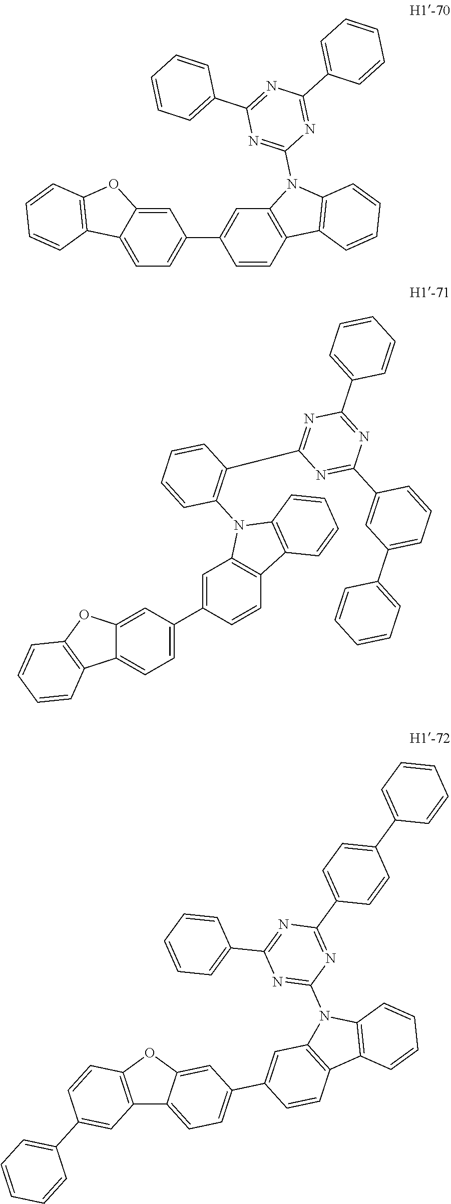 [SMILES: c1ccc(-c2nc(-c3ccccc3)nc(-n3c4ccccc4c4ccc(-c5ccc6c(c5)oc5ccccc56)cc43)n2)cc1 ; c1ccc(-c2cccc(-c3nc(-c4ccccc4)nc(-c4ccccc4-n4c5ccccc5c5ccc(-c6ccc7c(c6)oc6ccccc67)cc54)n3)c2)cc1 ; c1ccc(-c2ccc(-c3nc(-c4ccccc4)nc(-n4c5ccccc5c5ccc(-c6ccc7c(c6)oc6ccc(-c8ccccc8)cc67)cc54)n3)cc2)cc1]

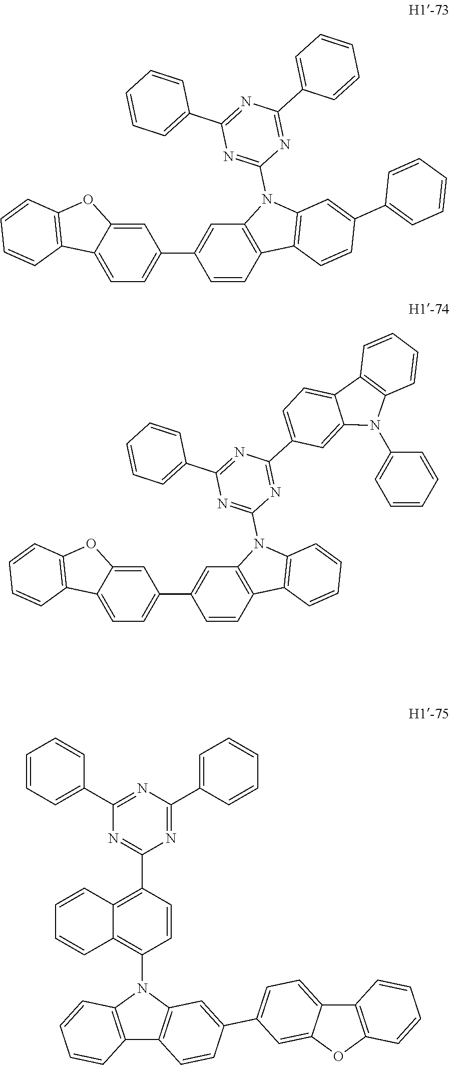 [SMILES: c1ccc(-c2ccc3c4ccc(-c5ccc6c(c5)oc5ccccc56)cc4n(-c4nc(-c5ccccc5)nc(-c5ccccc5)n4)c3c2)cc1 ; c1ccc(-c2nc(-c3ccc4c5ccccc5n(-c5ccccc5)c4c3)nc(-n3c4ccccc4c4ccc(-c5ccc6c(c5)oc5ccccc56)cc43)n2)cc1 ; c1ccc(-c2nc(-c3ccccc3)nc(-c3ccc(-n4c5ccccc5c5ccc(-c6ccc7c(c6)oc6ccccc67)cc54)c4ccccc34)n2)cc1]

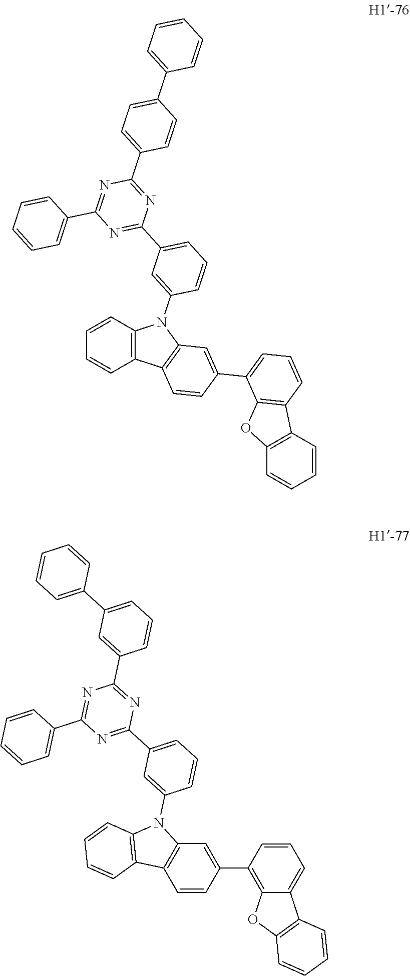 [SMILES: c1ccc(-c2ccc(-c3nc(-c4ccccc4)nc(-c4cccc(-n5c6ccccc6c6ccc(-c7cccc8c7oc7ccccc78)cc65)c4)n3)cc2)cc1 ; c1ccc(-c2cccc(-c3nc(-c4ccccc4)nc(-c4cccc(-n5c6ccccc6c6ccc(-c7cccc8c7oc7ccccc78)cc65)c4)n3)c2)cc1]

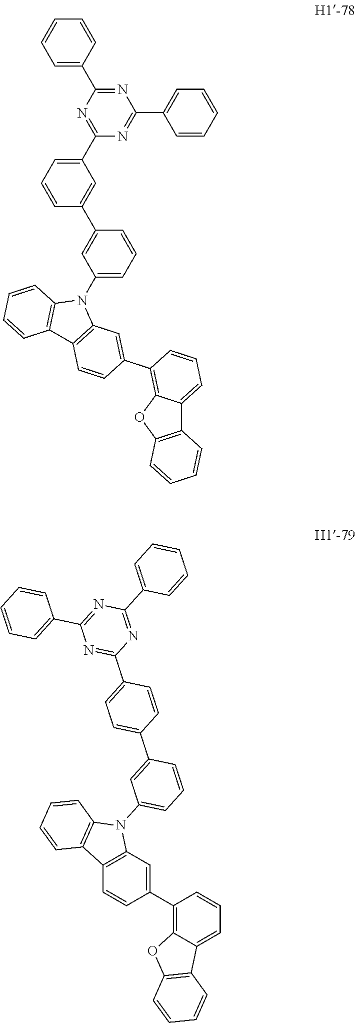 [SMILES: c1ccc(-c2nc(-c3ccccc3)nc(-c3cccc(-c4cccc(-n5c6ccccc6c6ccc(-c7cccc8c7oc7ccccc78)cc65)c4)c3)n2)cc1 ; c1ccc(-c2nc(-c3ccccc3)nc(-c3ccc(-c4cccc(-n5c6ccccc6c6ccc(-c7cccc8c7oc7ccccc78)cc65)c4)cc3)n2)cc1]

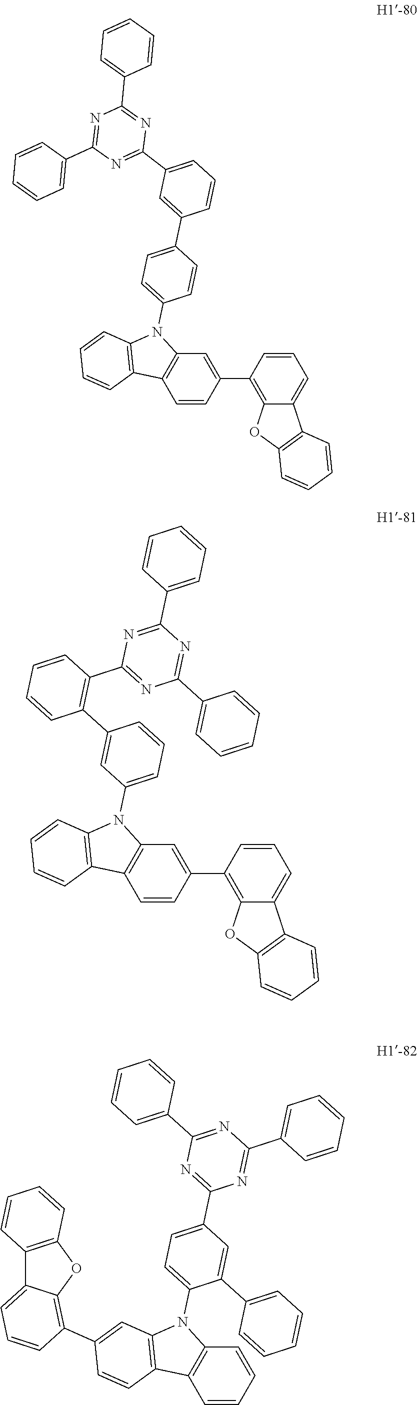 [SMILES: c1ccc(-c2nc(-c3ccccc3)nc(-c3cccc(-c4ccc(-n5c6ccccc6c6ccc(-c7cccc8c7oc7ccccc78)cc65)cc4)c3)n2)cc1 ; c1ccc(-c2nc(-c3ccccc3)nc(-c3ccccc3-c3cccc(-n4c5ccccc5c5ccc(-c6cccc7c6oc6ccccc67)cc54)c3)n2)cc1 ; c1ccc(-c2nc(-c3ccccc3)nc(-c3ccc(-n4c5ccccc5c5ccc(-c6cccc7c6oc6ccccc67)cc54)c(-c4ccccc4)c3)n2)cc1]

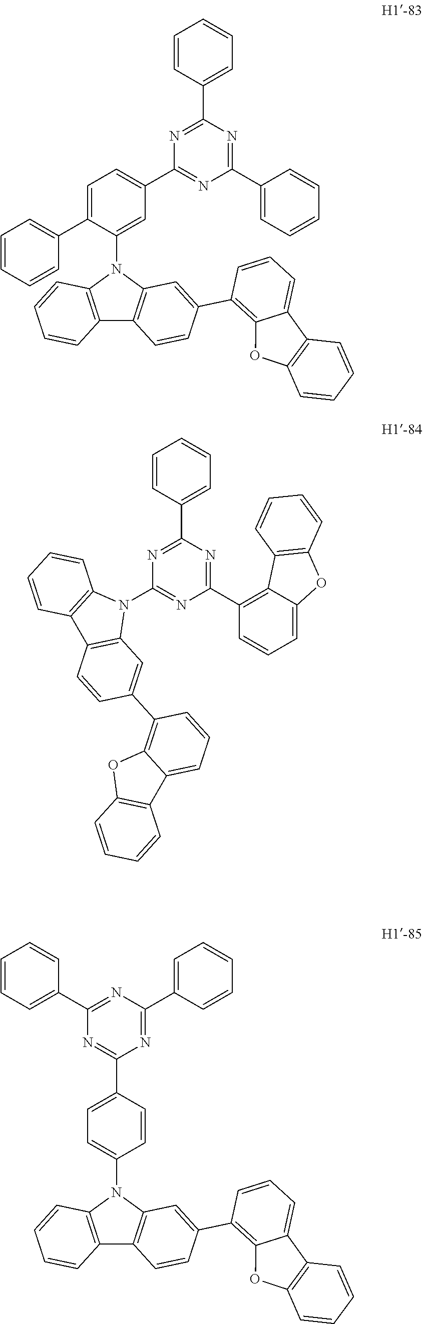 [SMILES: c1ccc(-c2nc(-c3ccccc3)nc(-c3ccc(-c4ccccc4)c(-n4c5ccccc5c5ccc(-c6cccc7c6oc6ccccc67)cc54)c3)n2)cc1 ; c1ccc(-c2nc(-c3cccc4oc5ccccc5c34)nc(-n3c4ccccc4c4ccc(-c5cccc6c5oc5ccccc56)cc43)n2)cc1 ; c1ccc(-c2nc(-c3ccccc3)nc(-c3ccc(-n4c5ccccc5c5ccc(-c6cccc7c6oc6ccccc67)cc54)cc3)n2)cc1]

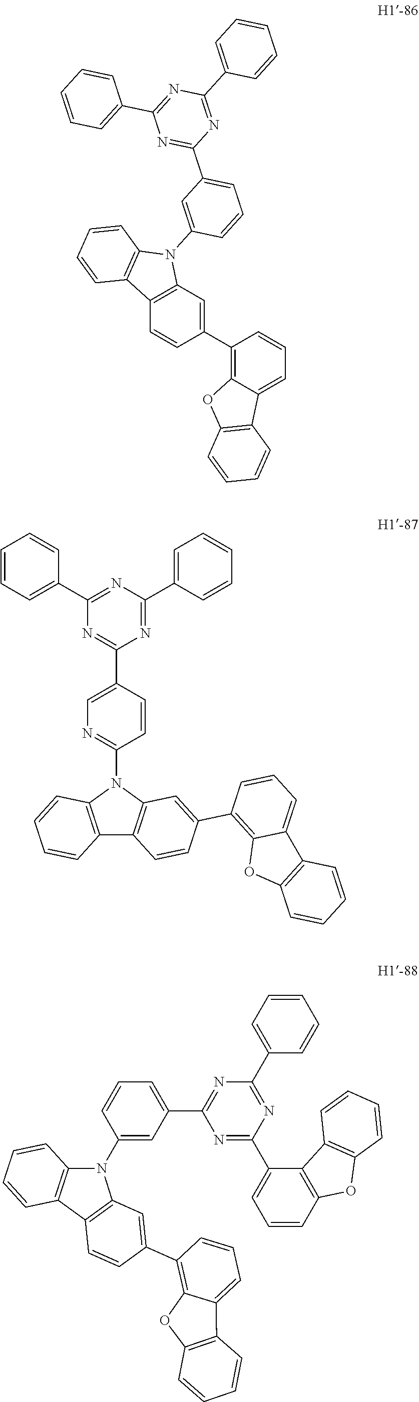 [SMILES: c1ccc(-c2nc(-c3ccccc3)nc(-c3cccc(-n4c5ccccc5c5ccc(-c6cccc7c6oc6ccccc67)cc54)c3)n2)cc1 ; c1ccc(-c2nc(-c3ccccc3)nc(-c3ccc(-n4c5ccccc5c5ccc(-c6cccc7c6oc6ccccc67)cc54)nc3)n2)cc1 ; c1ccc(-c2nc(-c3cccc(-n4c5ccccc5c5ccc(-c6cccc7c6oc6ccccc67)cc54)c3)nc(-c3cccc4oc5ccccc5c34)n2)cc1]

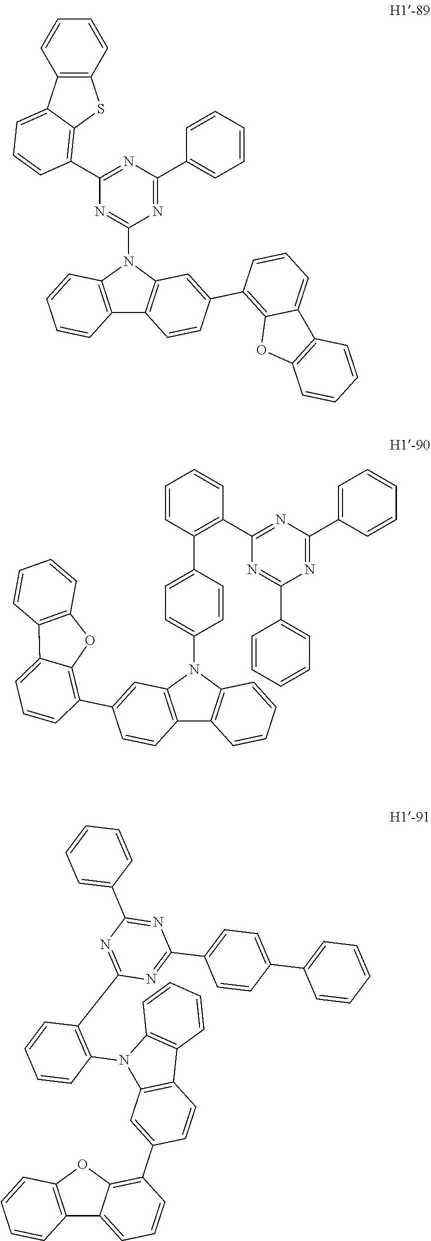 [SMILES: c1ccc(-c2nc(-c3cccc4c3sc3ccccc34)nc(-n3c4ccccc4c4ccc(-c5cccc6c5oc5ccccc56)cc43)n2)cc1 ; c1ccc(-c2nc(-c3ccccc3)nc(-c3ccccc3-c3ccc(-n4c5ccccc5c5ccc(-c6cccc7c6oc6ccccc67)cc54)cc3)n2)cc1 ; c1ccc(-c2ccc(-c3nc(-c4ccccc4)nc(-c4ccccc4-n4c5ccccc5c5ccc(-c6cccc7c6oc6ccccc67)cc54)n3)cc2)cc1]

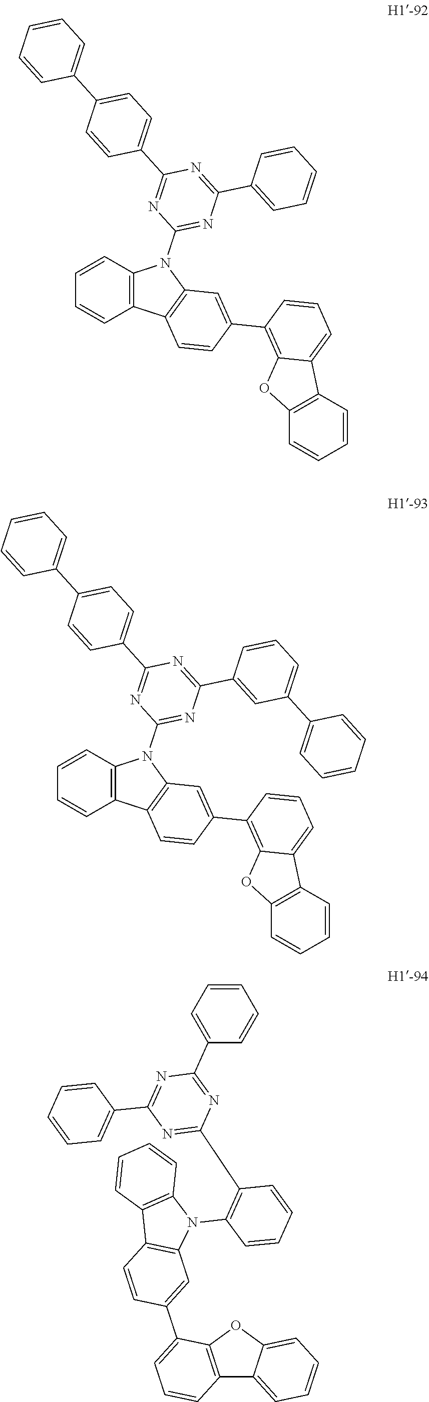 [SMILES: c1ccc(-c2ccc(-c3nc(-c4ccccc4)nc(-n4c5ccccc5c5ccc(-c6cccc7c6oc6ccccc67)cc54)n3)cc2)cc1 ; c1ccc(-c2ccc(-c3nc(-c4cccc(-c5ccccc5)c4)nc(-n4c5ccccc5c5ccc(-c6cccc7c6oc6ccccc67)cc54)n3)cc2)cc1 ; c1ccc(-c2nc(-c3ccccc3)nc(-c3ccccc3-n3c4ccccc4c4ccc(-c5cccc6c5oc5ccccc56)cc43)n2)cc1]

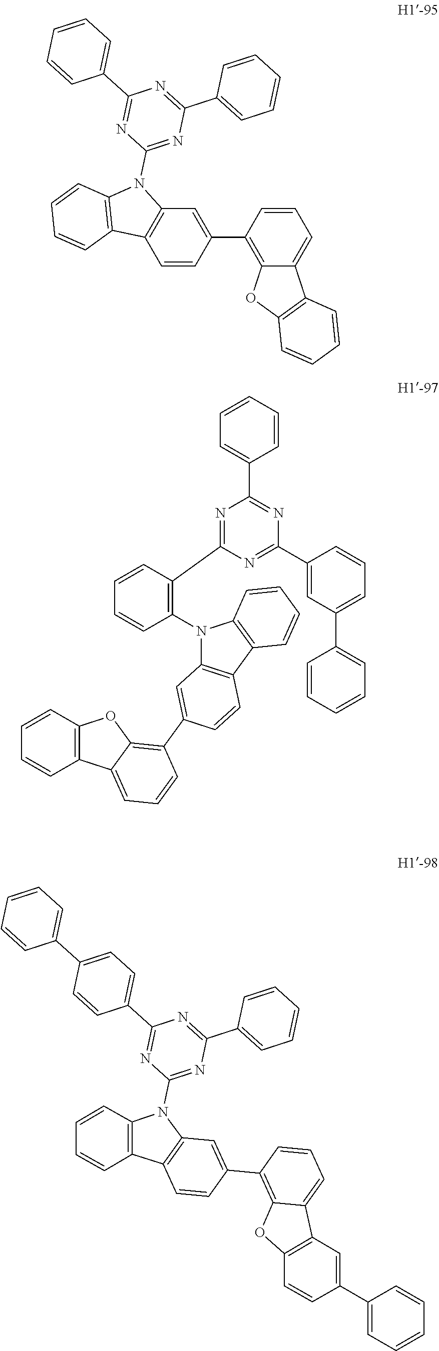 [SMILES: c1ccc(-c2nc(-c3ccccc3)nc(-n3c4ccccc4c4ccc(-c5cccc6c5oc5ccccc56)cc43)n2)cc1 ; c1ccc(-c2cccc(-c3nc(-c4ccccc4)nc(-c4ccccc4-n4c5ccccc5c5ccc(-c6cccc7c6oc6ccccc67)cc54)n3)c2)cc1 ; c1ccc(-c2ccc(-c3nc(-c4ccccc4)nc(-n4c5ccccc5c5ccc(-c6cccc7c6oc6ccc(-c8ccccc8)cc67)cc54)n3)cc2)cc1]

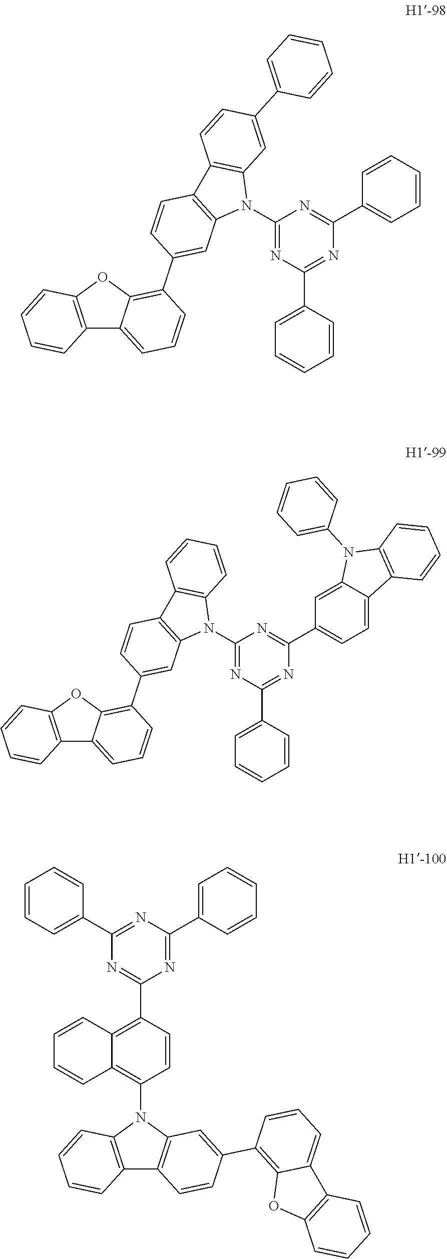 [SMILES: c1ccc(-c2ccc3c4ccc(-c5cccc6c5oc5ccccc56)cc4n(-c4nc(-c5ccccc5)nc(-c5ccccc5)n4)c3c2)cc1 ; c1ccc(-c2nc(-c3ccc4c5ccccc5n(-c5ccccc5)c4c3)nc(-n3c4ccccc4c4ccc(-c5cccc6c5oc5ccccc56)cc43)n2)cc1 ; c1ccc(-c2nc(-c3ccccc3)nc(-c3ccc(-n4c5ccccc5c5ccc(-c6cccc7c6oc6ccccc67)cc54)c4ccccc34)n2)cc1]

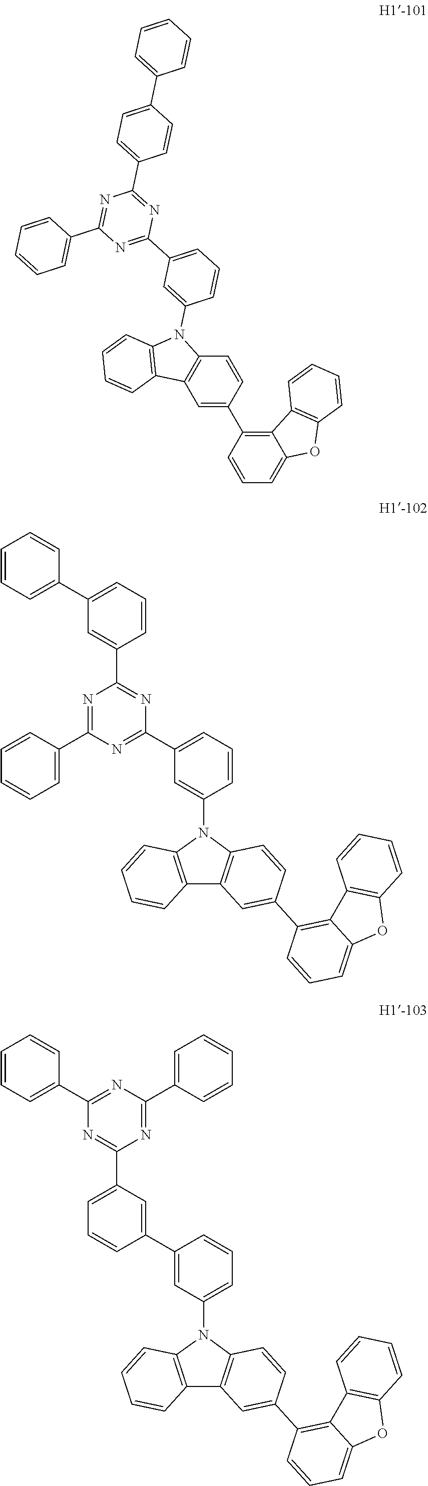 [SMILES: c1ccc(-c2ccc(-c3nc(-c4ccccc4)nc(-c4cccc(-n5c6ccccc6c6cc(-c7cccc8oc9ccccc9c78)ccc65)c4)n3)cc2)cc1 ; c1ccc(-c2cccc(-c3nc(-c4ccccc4)nc(-c4cccc(-n5c6ccccc6c6cc(-c7cccc8oc9ccccc9c78)ccc65)c4)n3)c2)cc1 ; c1ccc(-c2nc(-c3ccccc3)nc(-c3cccc(-c4cccc(-n5c6ccccc6c6cc(-c7cccc8oc9ccccc9c78)ccc65)c4)c3)n2)cc1]

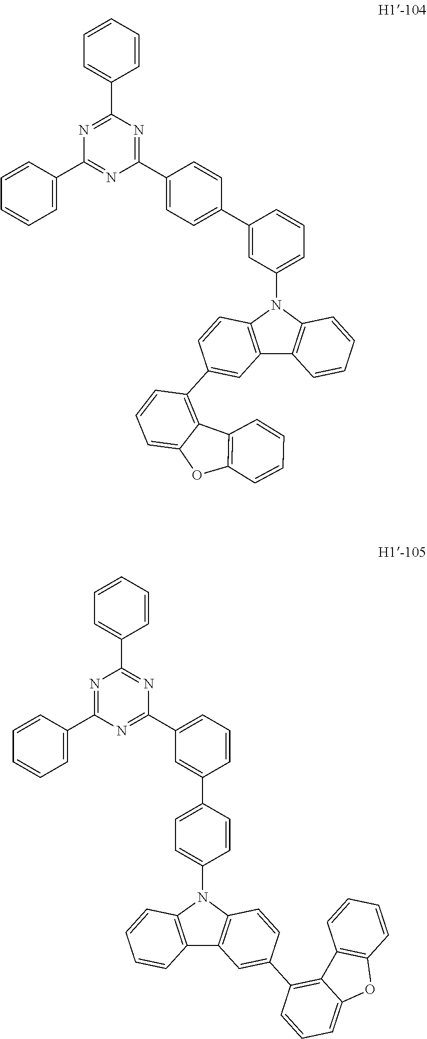 [SMILES: c1ccc(-c2nc(-c3ccccc3)nc(-c3ccc(-c4cccc(-n5c6ccccc6c6cc(-c7cccc8oc9ccccc9c78)ccc65)c4)cc3)n2)cc1 ; c1ccc(-c2nc(-c3ccccc3)nc(-c3cccc(-c4ccc(-n5c6ccccc6c6cc(-c7cccc8oc9ccccc9c78)ccc65)cc4)c3)n2)cc1]

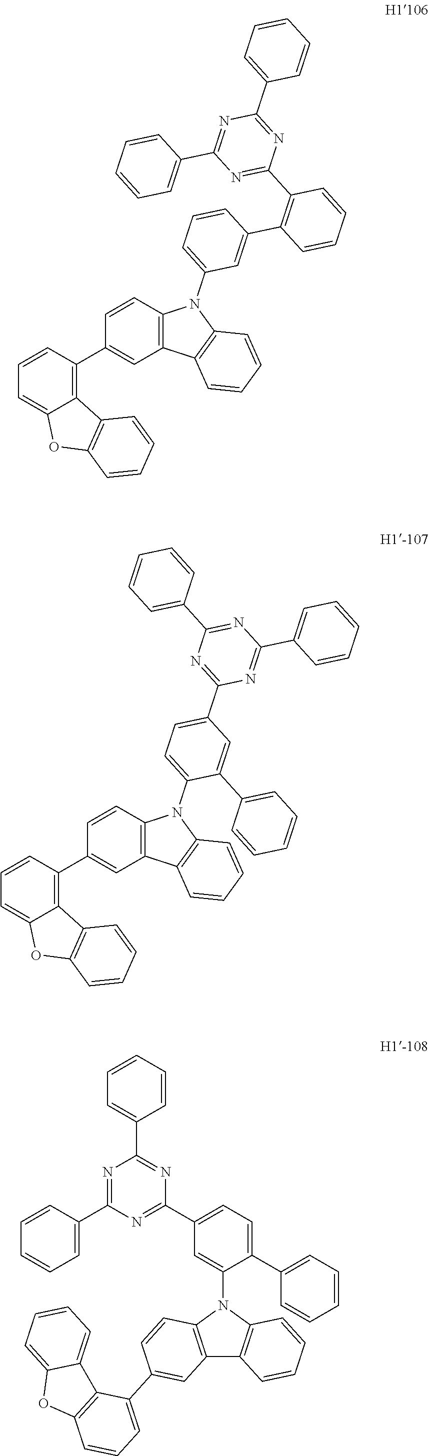 [SMILES: c1ccc(-c2nc(-c3ccccc3)nc(-c3ccccc3-c3cccc(-n4c5ccccc5c5cc(-c6cccc7oc8ccccc8c67)ccc54)c3)n2)cc1 ; c1ccc(-c2nc(-c3ccccc3)nc(-c3ccc(-n4c5ccccc5c5cc(-c6cccc7oc8ccccc8c67)ccc54)c(-c4ccccc4)c3)n2)cc1 ; c1ccc(-c2nc(-c3ccccc3)nc(-c3ccc(-c4ccccc4)c(-n4c5ccccc5c5cc(-c6cccc7oc8ccccc8c67)ccc54)c3)n2)cc1]

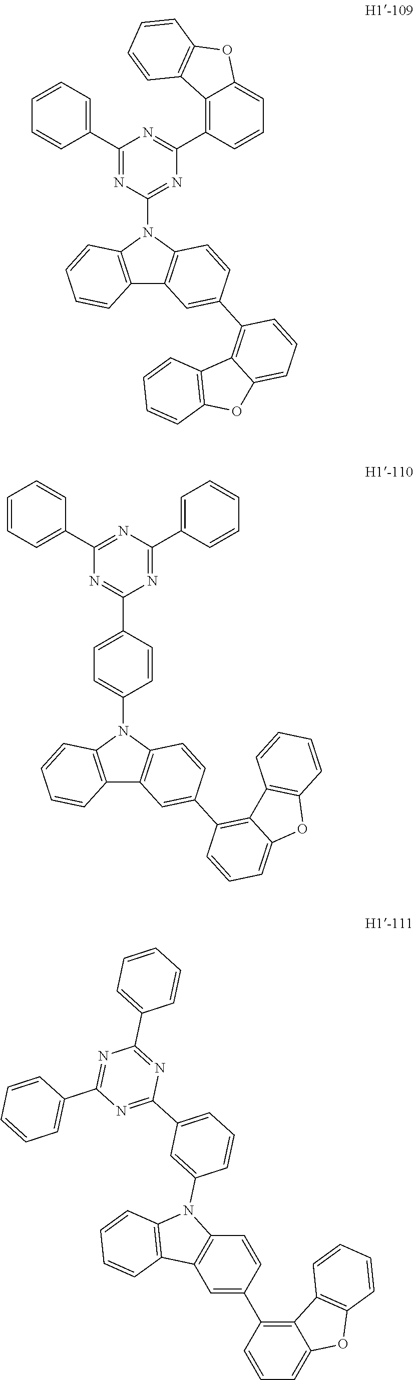 [SMILES: c1ccc(-c2nc(-c3cccc4oc5ccccc5c34)nc(-n3c4ccccc4c4cc(-c5cccc6oc7ccccc7c56)ccc43)n2)cc1 ; c1ccc(-c2nc(-c3ccccc3)nc(-c3ccc(-n4c5ccccc5c5cc(-c6cccc7oc8ccccc8c67)ccc54)cc3)n2)cc1 ; c1ccc(-c2nc(-c3ccccc3)nc(-c3cccc(-n4c5ccccc5c5cc(-c6cccc7oc8ccccc8c67)ccc54)c3)n2)cc1]

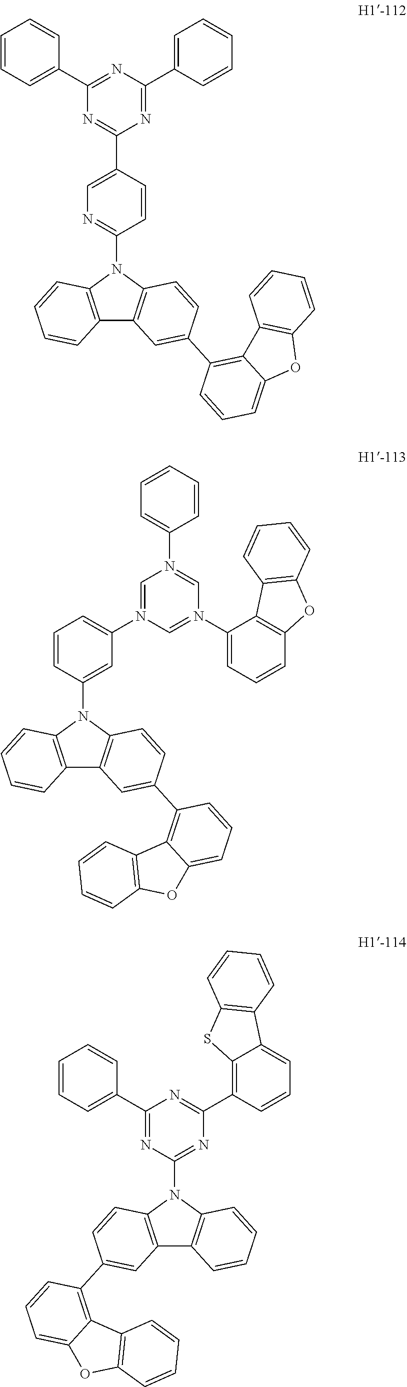

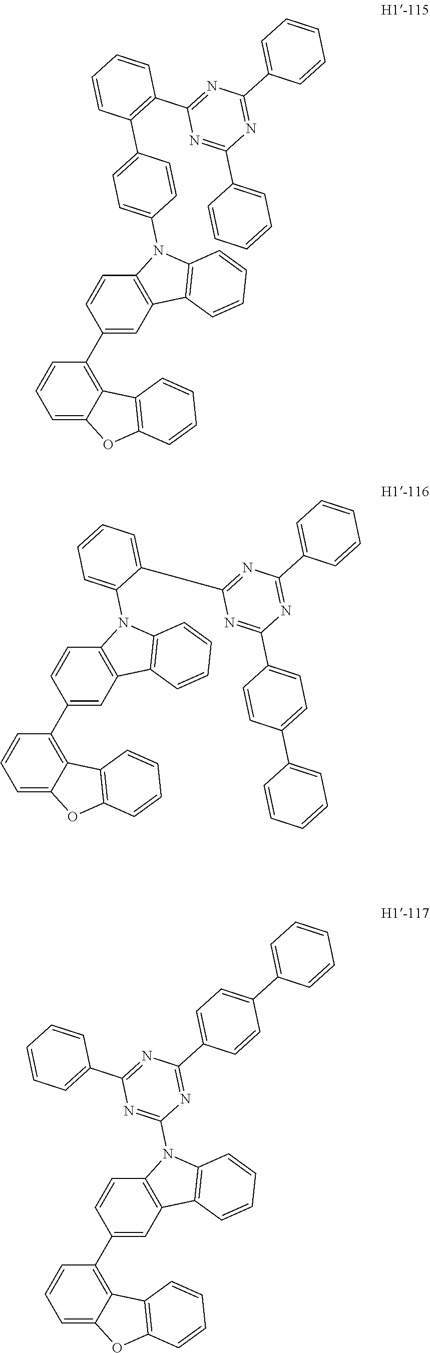 [SMILES: c1ccc(-c2nc(-c3ccccc3)nc(-c3ccccc3-c3ccc(-n4c5ccccc5c5cc(-c6cccc7oc8ccccc8c67)ccc54)cc3)n2)cc1 ; c1ccc(-c2ccc(-c3nc(-c4ccccc4)nc(-c4ccccc4-n4c5ccccc5c5cc(-c6cccc7oc8ccccc8c67)ccc54)n3)cc2)cc1 ; c1ccc(-c2ccc(-c3nc(-c4ccccc4)nc(-n4c5ccccc5c5cc(-c6cccc7oc8ccccc8c67)ccc54)n3)cc2)cc1]

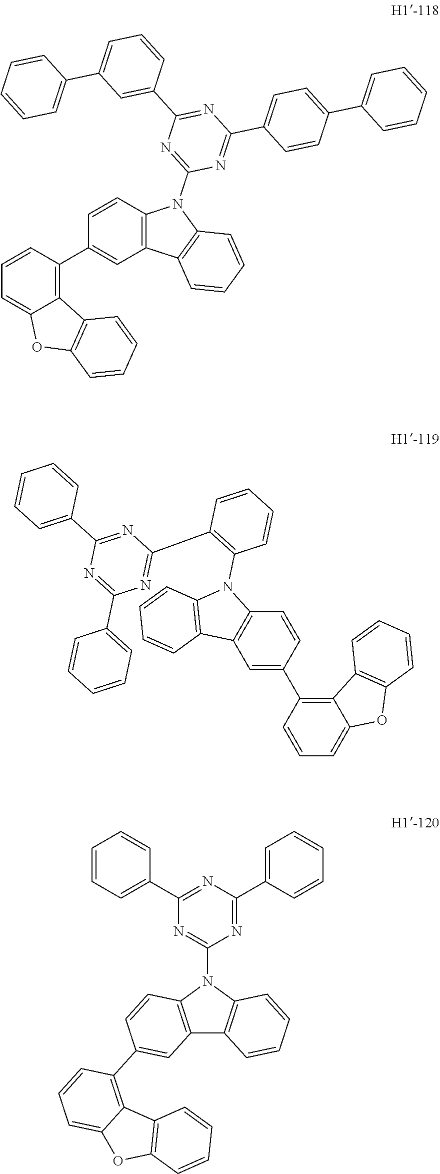 [SMILES: c1ccc(-c2ccc(-c3nc(-c4cccc(-c5ccccc5)c4)nc(-n4c5ccccc5c5cc(-c6cccc7oc8ccccc8c67)ccc54)n3)cc2)cc1 ; c1ccc(-c2nc(-c3ccccc3)nc(-c3ccccc3-n3c4ccccc4c4cc(-c5cccc6oc7ccccc7c56)ccc43)n2)cc1 ; c1ccc(-c2nc(-c3ccccc3)nc(-n3c4ccccc4c4cc(-c5cccc6oc7ccccc7c56)ccc43)n2)cc1]

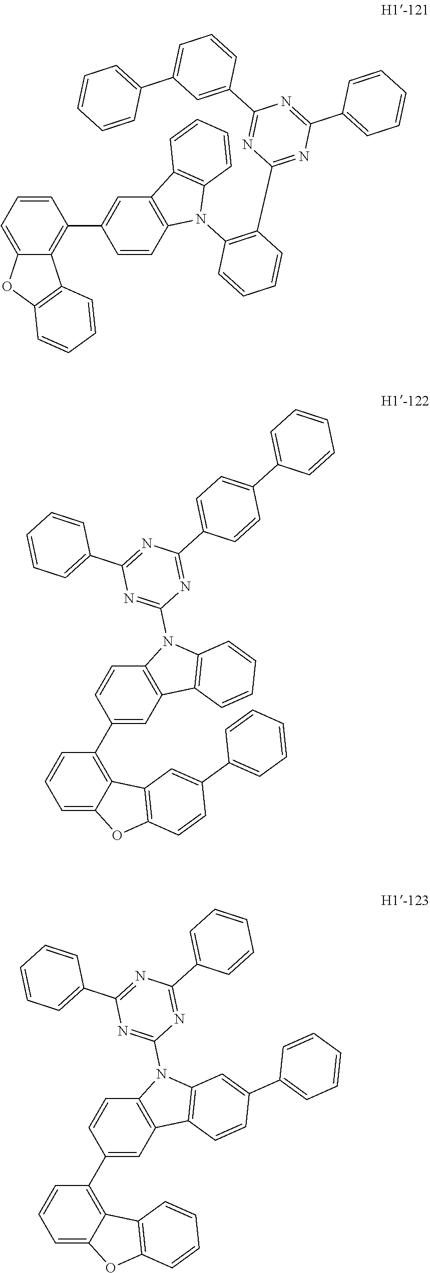 [SMILES: c1ccc(-c2cccc(-c3nc(-c4ccccc4)nc(-c4ccccc4-n4c5ccccc5c5cc(-c6cccc7oc8ccccc8c67)ccc54)n3)c2)cc1 ; c1ccc(-c2ccc(-c3nc(-c4ccccc4)nc(-n4c5ccccc5c5cc(-c6cccc7oc8ccc(-c9ccccc9)cc8c67)ccc54)n3)cc2)cc1 ; c1ccc(-c2ccc3c4cc(-c5cccc6oc7ccccc7c56)ccc4n(-c4nc(-c5ccccc5)nc(-c5ccccc5)n4)c3c2)cc1]

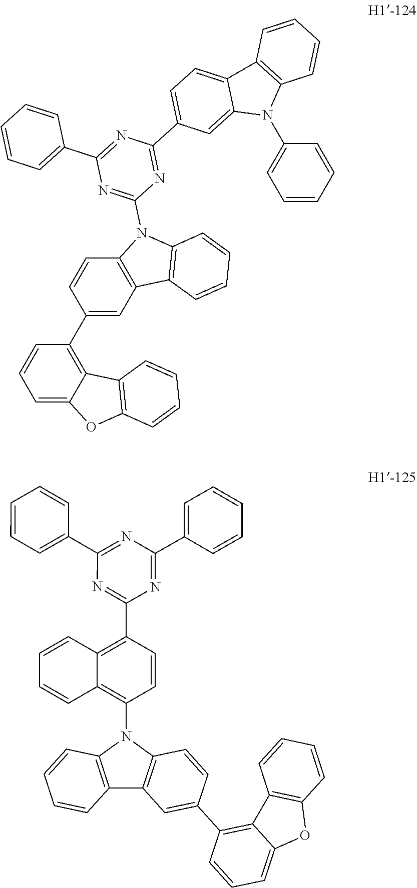 [SMILES: c1ccc(-c2nc(-c3ccc4c5ccccc5n(-c5ccccc5)c4c3)nc(-n3c4ccccc4c4cc(-c5cccc6oc7ccccc7c56)ccc43)n2)cc1 ; c1ccc(-c2nc(-c3ccccc3)nc(-c3ccc(-n4c5ccccc5c5cc(-c6cccc7oc8ccccc8c67)ccc54)c4ccccc34)n2)cc1]

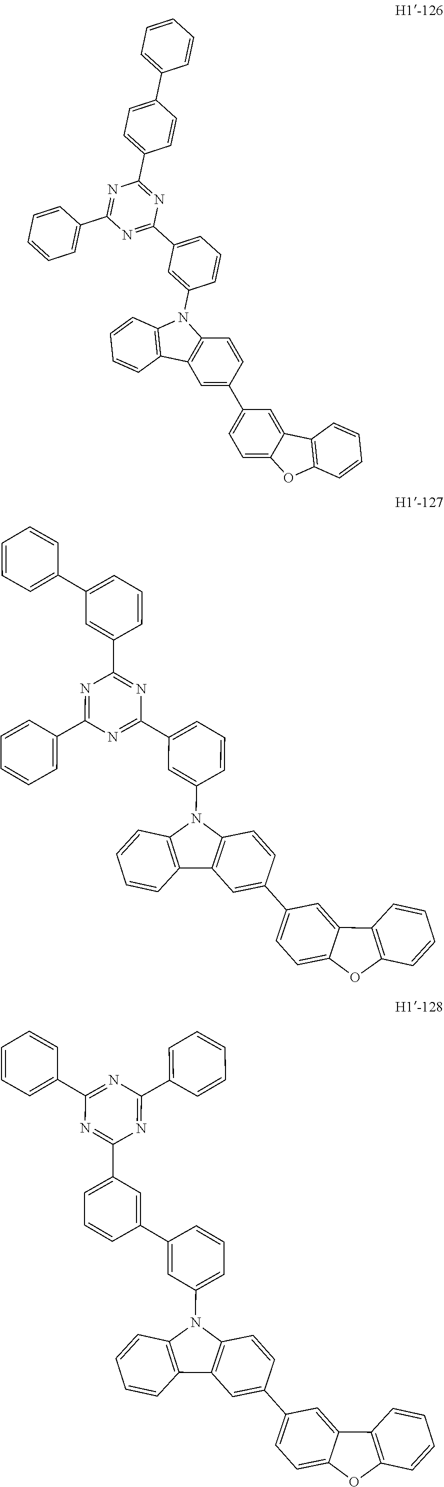 [SMILES: c1ccc(-c2ccc(-c3nc(-c4ccccc4)nc(-c4cccc(-n5c6ccccc6c6cc(-c7ccc8oc9ccccc9c8c7)ccc65)c4)n3)cc2)cc1 ; c1ccc(-c2cccc(-c3nc(-c4ccccc4)nc(-c4cccc(-n5c6ccccc6c6cc(-c7ccc8oc9ccccc9c8c7)ccc65)c4)n3)c2)cc1 ; c1ccc(-c2nc(-c3ccccc3)nc(-c3cccc(-c4cccc(-n5c6ccccc6c6cc(-c7ccc8oc9ccccc9c8c7)ccc65)c4)c3)n2)cc1]

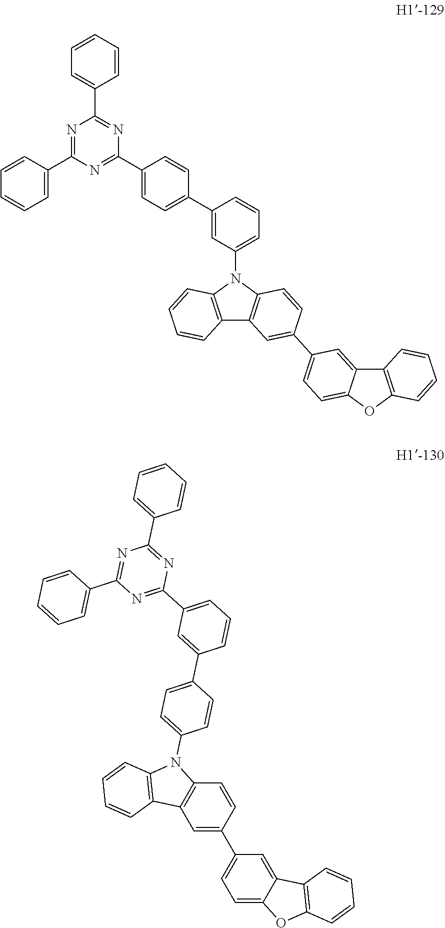 [SMILES: c1ccc(-c2nc(-c3ccccc3)nc(-c3ccc(-c4cccc(-n5c6ccccc6c6cc(-c7ccc8oc9ccccc9c8c7)ccc65)c4)cc3)n2)cc1 ; c1ccc(-c2nc(-c3ccccc3)nc(-c3cccc(-c4ccc(-n5c6ccccc6c6cc(-c7ccc8oc9ccccc9c8c7)ccc65)cc4)c3)n2)cc1]

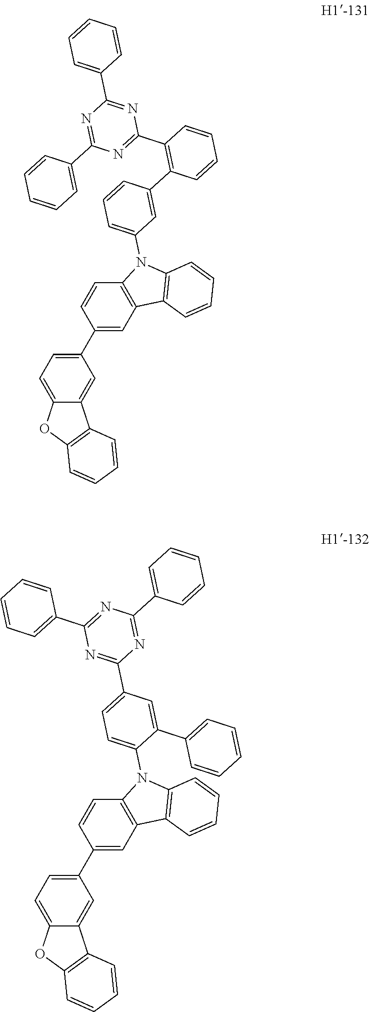 [SMILES: c1ccc(-c2nc(-c3ccccc3)nc(-c3ccccc3-c3cccc(-n4c5ccccc5c5cc(-c6ccc7oc8ccccc8c7c6)ccc54)c3)n2)cc1 ; c1ccc(-c2nc(-c3ccccc3)nc(-c3ccc(-n4c5ccccc5c5cc(-c6ccc7oc8ccccc8c7c6)ccc54)c(-c4ccccc4)c3)n2)cc1]

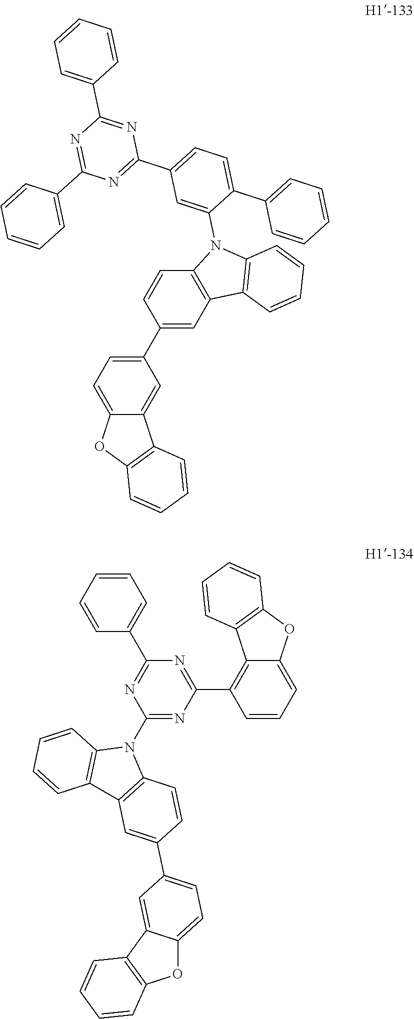 [SMILES: c1ccc(-c2nc(-c3ccccc3)nc(-c3ccc(-c4ccccc4)c(-n4c5ccccc5c5cc(-c6ccc7oc8ccccc8c7c6)ccc54)c3)n2)cc1 ; c1ccc(-c2nc(-c3cccc4oc5ccccc5c34)nc(-n3c4ccccc4c4cc(-c5ccc6oc7ccccc7c6c5)ccc43)n2)cc1]

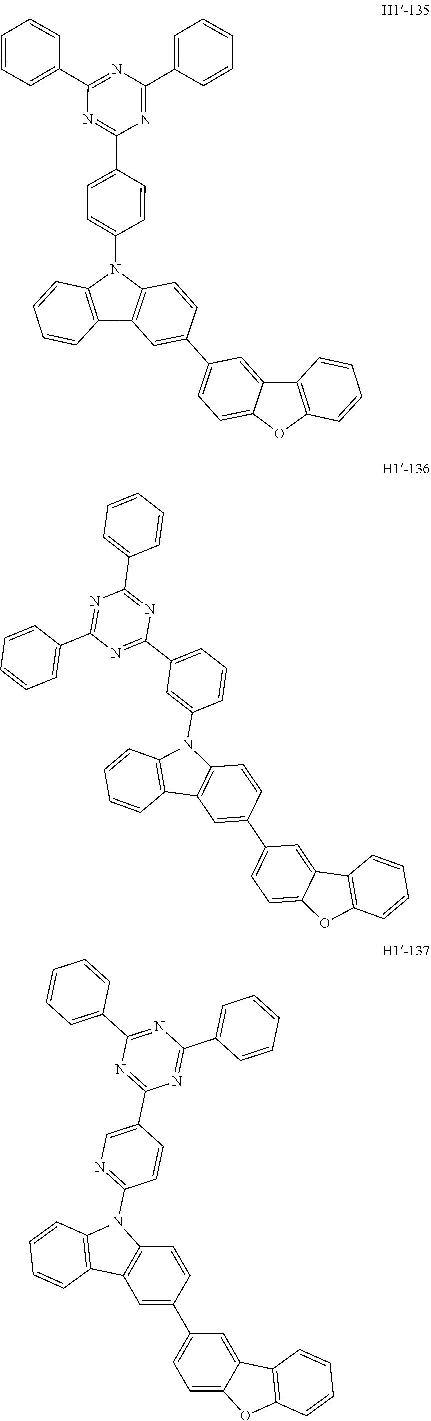 [SMILES: c1ccc(-c2nc(-c3ccccc3)nc(-c3ccc(-n4c5ccccc5c5cc(-c6ccc7oc8ccccc8c7c6)ccc54)cc3)n2)cc1 ; c1ccc(-c2nc(-c3ccccc3)nc(-c3cccc(-n4c5ccccc5c5cc(-c6ccc7oc8ccccc8c7c6)ccc54)c3)n2)cc1 ; c1ccc(-c2nc(-c3ccccc3)nc(-c3ccc(-n4c5ccccc5c5cc(-c6ccc7oc8ccccc8c7c6)ccc54)nc3)n2)cc1]

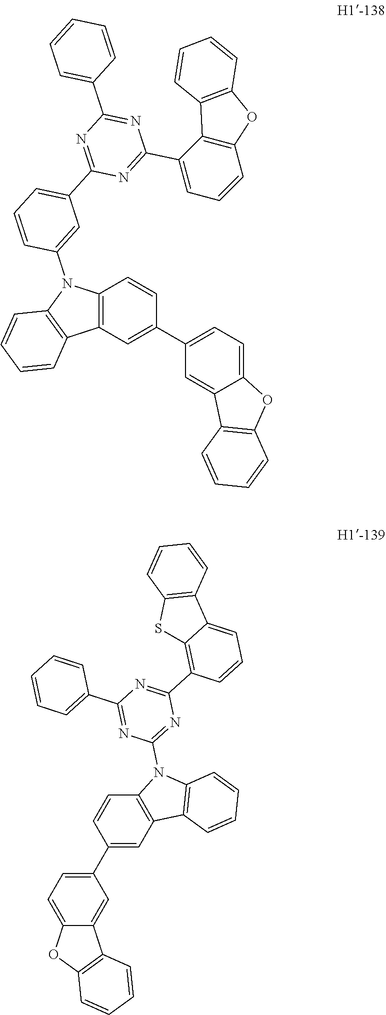 [SMILES: c1ccc(-c2nc(-c3cccc(-n4c5ccccc5c5cc(-c6ccc7oc8ccccc8c7c6)ccc54)c3)nc(-c3cccc4oc5ccccc5c34)n2)cc1 ; c1ccc(-c2nc(-c3cccc4c3sc3ccccc34)nc(-n3c4ccccc4c4cc(-c5ccc6oc7ccccc7c6c5)ccc43)n2)cc1]

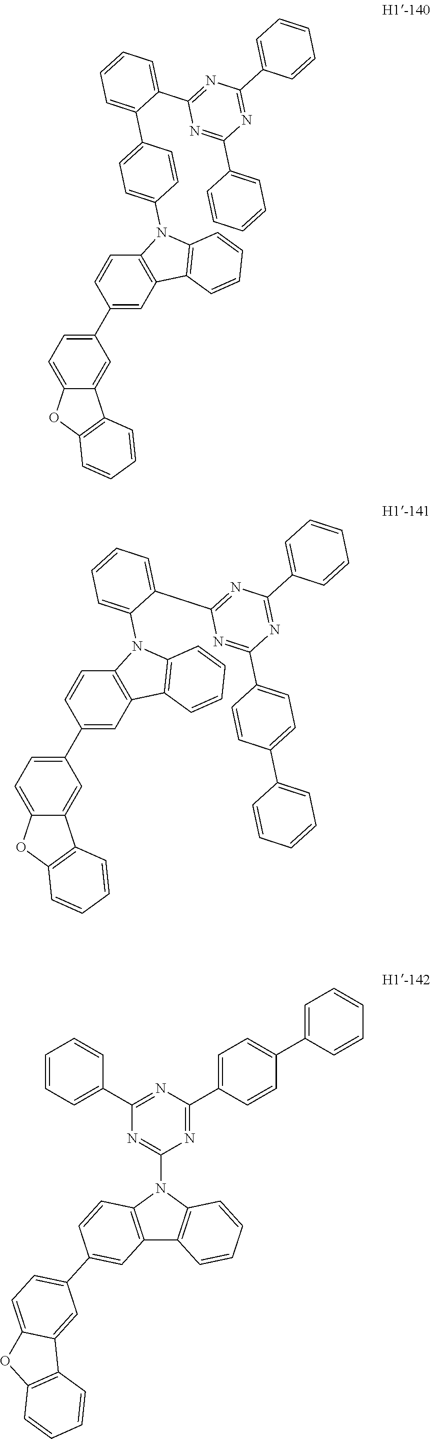 [SMILES: c1ccc(-c2nc(-c3ccccc3)nc(-c3ccccc3-c3ccc(-n4c5ccccc5c5cc(-c6ccc7oc8ccccc8c7c6)ccc54)cc3)n2)cc1 ; c1ccc(-c2ccc(-c3nc(-c4ccccc4)nc(-c4ccccc4-n4c5ccccc5c5cc(-c6ccc7oc8ccccc8c7c6)ccc54)n3)cc2)cc1 ; c1ccc(-c2ccc(-c3nc(-c4ccccc4)nc(-n4c5ccccc5c5cc(-c6ccc7oc8ccccc8c7c6)ccc54)n3)cc2)cc1]

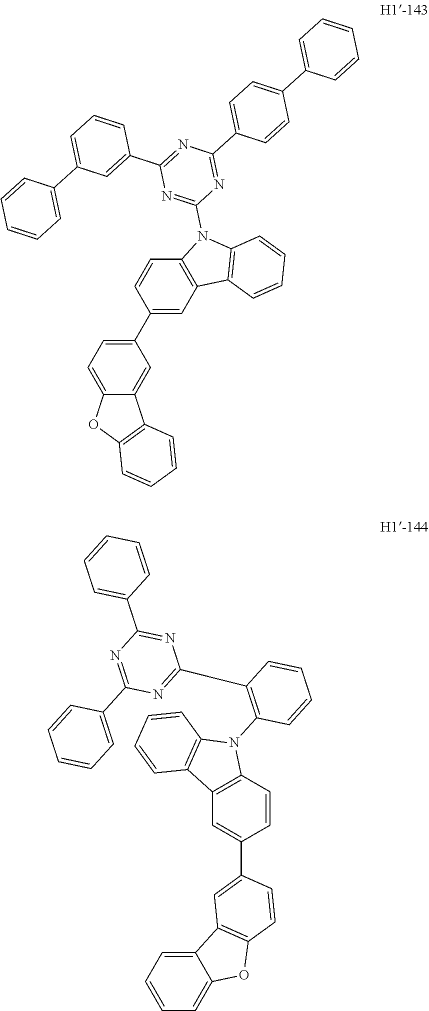 [SMILES: c1ccc(-c2ccc(-c3nc(-c4cccc(-c5ccccc5)c4)nc(-n4c5ccccc5c5cc(-c6ccc7oc8ccccc8c7c6)ccc54)n3)cc2)cc1 ; c1ccc(-c2nc(-c3ccccc3)nc(-c3ccccc3-n3c4ccccc4c4cc(-c5ccc6oc7ccccc7c6c5)ccc43)n2)cc1]

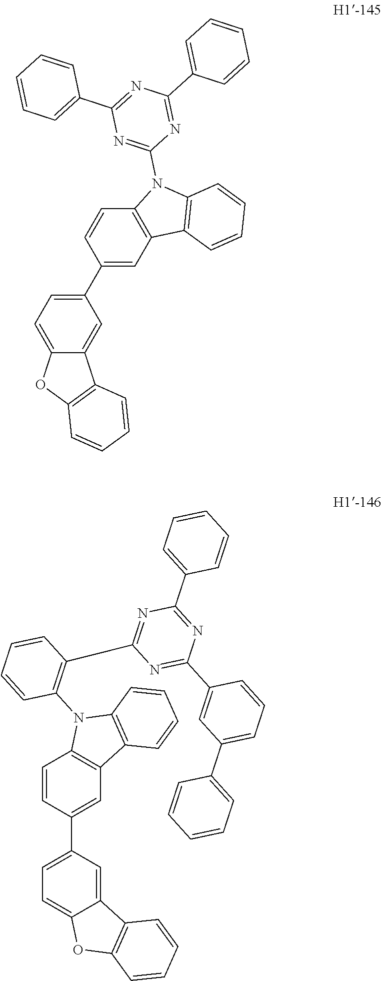 [SMILES: c1ccc(-c2nc(-c3ccccc3)nc(-n3c4ccccc4c4cc(-c5ccc6oc7ccccc7c6c5)ccc43)n2)cc1 ; c1ccc(-c2cccc(-c3nc(-c4ccccc4)nc(-c4ccccc4-n4c5ccccc5c5cc(-c6ccc7oc8ccccc8c7c6)ccc54)n3)c2)cc1]

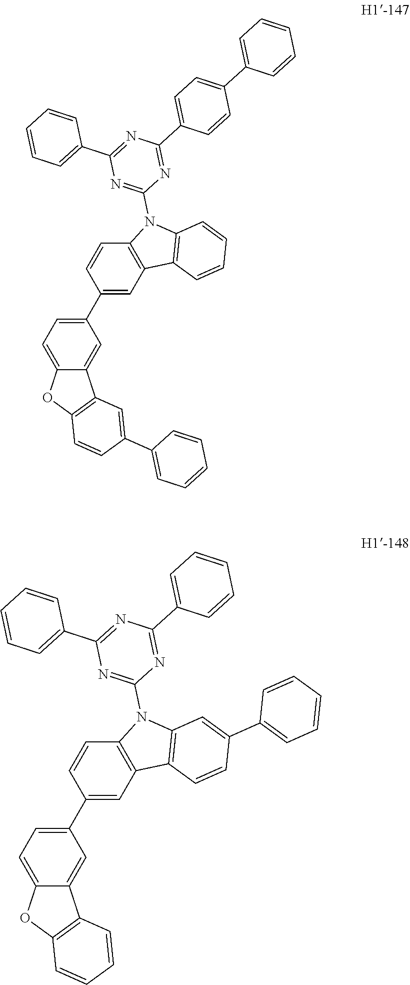 [SMILES: c1ccc(-c2ccc(-c3nc(-c4ccccc4)nc(-n4c5ccccc5c5cc(-c6ccc7oc8ccc(-c9ccccc9)cc8c7c6)ccc54)n3)cc2)cc1 ; c1ccc(-c2ccc3c4cc(-c5ccc6oc7ccccc7c6c5)ccc4n(-c4nc(-c5ccccc5)nc(-c5ccccc5)n4)c3c2)cc1]

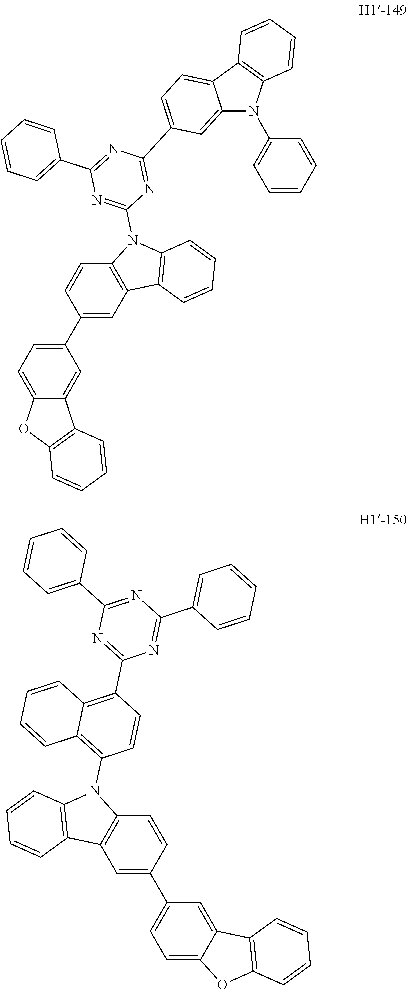

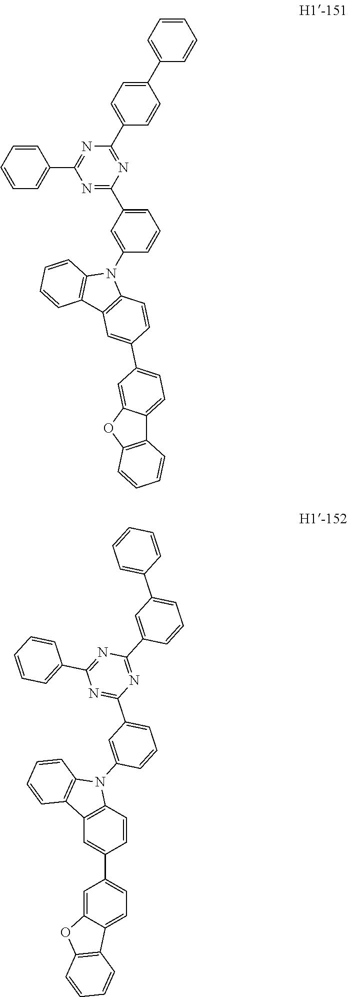 [SMILES: c1ccc(-c2ccc(-c3nc(-c4ccccc4)nc(-c4cccc(-n5c6ccccc6c6cc(-c7ccc8c(c7)oc7ccccc78)ccc65)c4)n3)cc2)cc1 ; c1ccc(-c2cccc(-c3nc(-c4ccccc4)nc(-c4cccc(-n5c6ccccc6c6cc(-c7ccc8c(c7)oc7ccccc78)ccc65)c4)n3)c2)cc1]

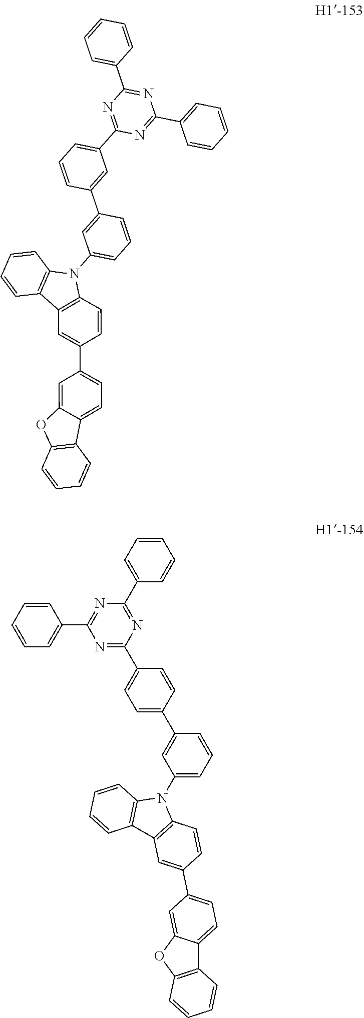 [SMILES: c1ccc(-c2nc(-c3ccccc3)nc(-c3cccc(-c4cccc(-n5c6ccccc6c6cc(-c7ccc8c(c7)oc7ccccc78)ccc65)c4)c3)n2)cc1 ; c1ccc(-c2nc(-c3ccccc3)nc(-c3ccc(-c4cccc(-n5c6ccccc6c6cc(-c7ccc8c(c7)oc7ccccc78)ccc65)c4)cc3)n2)cc1]

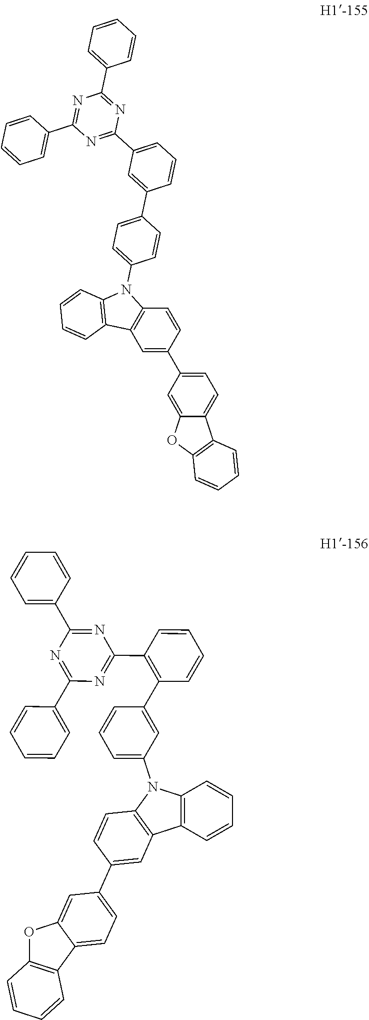 [SMILES: c1ccc(-c2nc(-c3ccccc3)nc(-c3cccc(-c4ccc(-n5c6ccccc6c6cc(-c7ccc8c(c7)oc7ccccc78)ccc65)cc4)c3)n2)cc1 ; c1ccc(-c2nc(-c3ccccc3)nc(-c3ccccc3-c3cccc(-n4c5ccccc5c5cc(-c6ccc7c(c6)oc6ccccc67)ccc54)c3)n2)cc1]

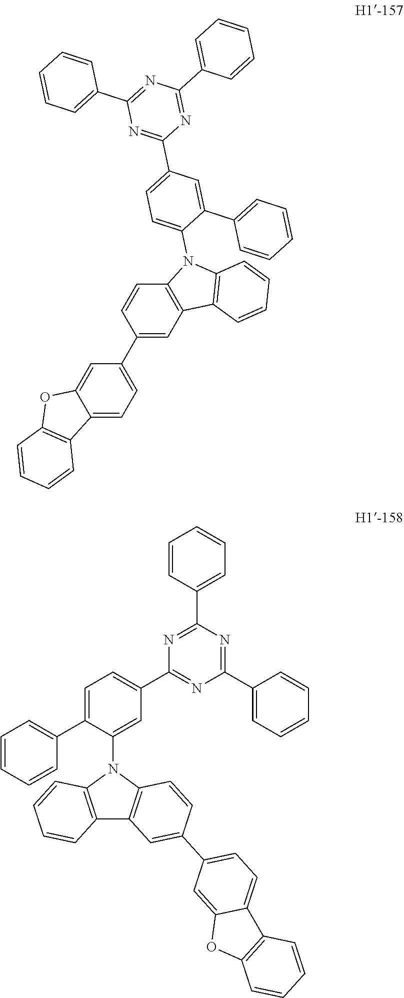 [SMILES: c1ccc(-c2nc(-c3ccccc3)nc(-c3ccc(-n4c5ccccc5c5cc(-c6ccc7c(c6)oc6ccccc67)ccc54)c(-c4ccccc4)c3)n2)cc1 ; c1ccc(-c2nc(-c3ccccc3)nc(-c3ccc(-c4ccccc4)c(-n4c5ccccc5c5cc(-c6ccc7c(c6)oc6ccccc67)ccc54)c3)n2)cc1]

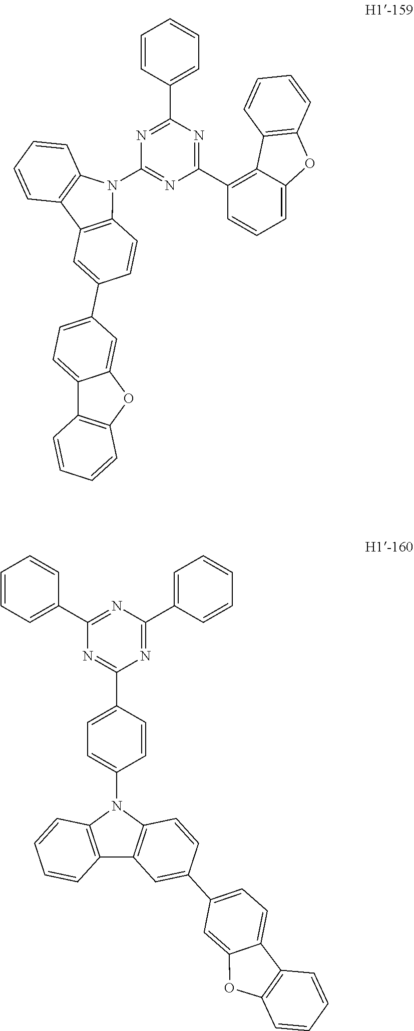 [SMILES: c1ccc(-c2nc(-c3cccc4oc5ccccc5c34)nc(-n3c4ccccc4c4cc(-c5ccc6c(c5)oc5ccccc56)ccc43)n2)cc1 ; c1ccc(-c2nc(-c3ccccc3)nc(-c3ccc(-n4c5ccccc5c5cc(-c6ccc7c(c6)oc6ccccc67)ccc54)cc3)n2)cc1]

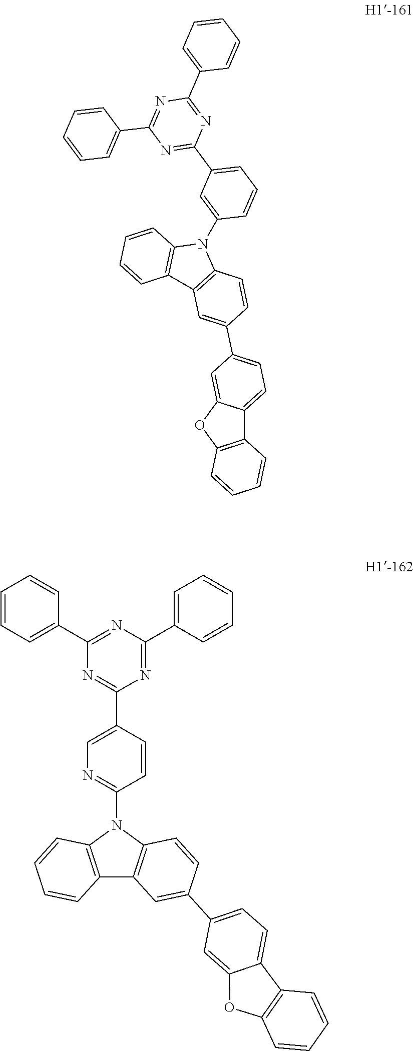 [SMILES: c1ccc(-c2nc(-c3ccccc3)nc(-c3cccc(-n4c5ccccc5c5cc(-c6ccc7c(c6)oc6ccccc67)ccc54)c3)n2)cc1 ; c1ccc(-c2nc(-c3ccccc3)nc(-c3ccc(-n4c5ccccc5c5cc(-c6ccc7c(c6)oc6ccccc67)ccc54)nc3)n2)cc1]

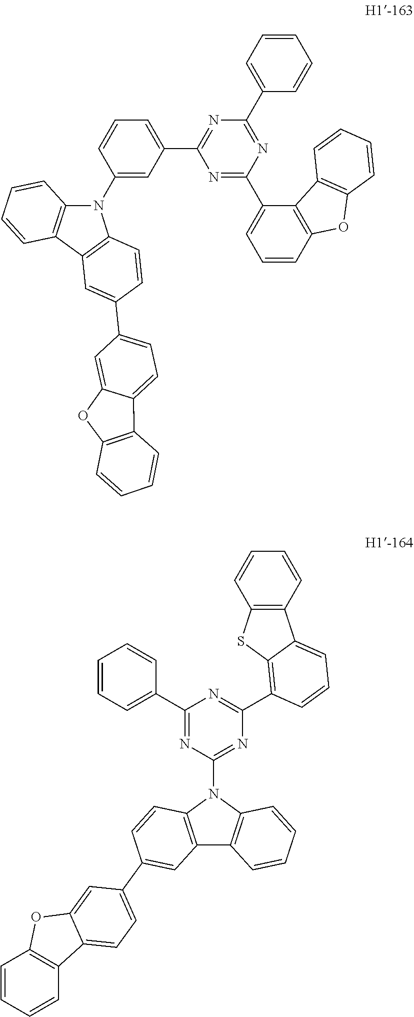 [SMILES: c1ccc(-c2nc(-c3cccc(-n4c5ccccc5c5cc(-c6ccc7c(c6)oc6ccccc67)ccc54)c3)nc(-c3cccc4oc5ccccc5c34)n2)cc1 ; c1ccc(-c2nc(-c3cccc4c3sc3ccccc34)nc(-n3c4ccccc4c4cc(-c5ccc6c(c5)oc5ccccc56)ccc43)n2)cc1]

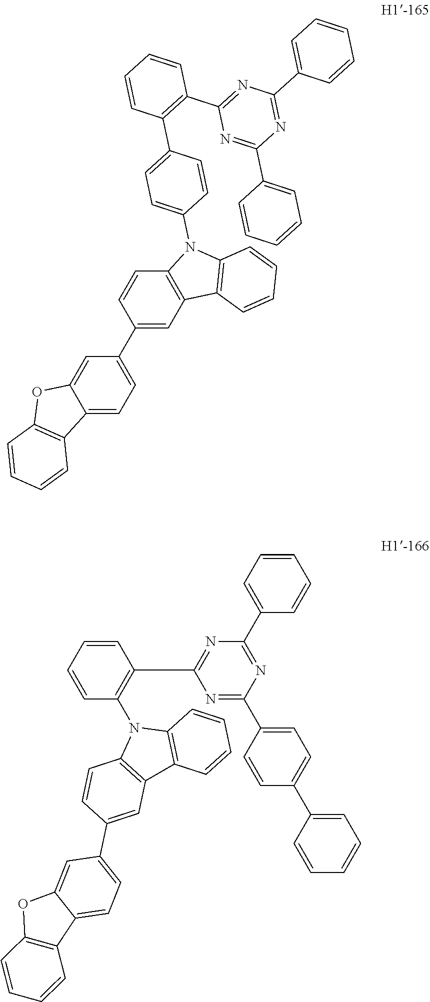 [SMILES: c1ccc(-c2nc(-c3ccccc3)nc(-c3ccccc3-c3ccc(-n4c5ccccc5c5cc(-c6ccc7c(c6)oc6ccccc67)ccc54)cc3)n2)cc1 ; c1ccc(-c2ccc(-c3nc(-c4ccccc4)nc(-c4ccccc4-n4c5ccccc5c5cc(-c6ccc7c(c6)oc6ccccc67)ccc54)n3)cc2)cc1]

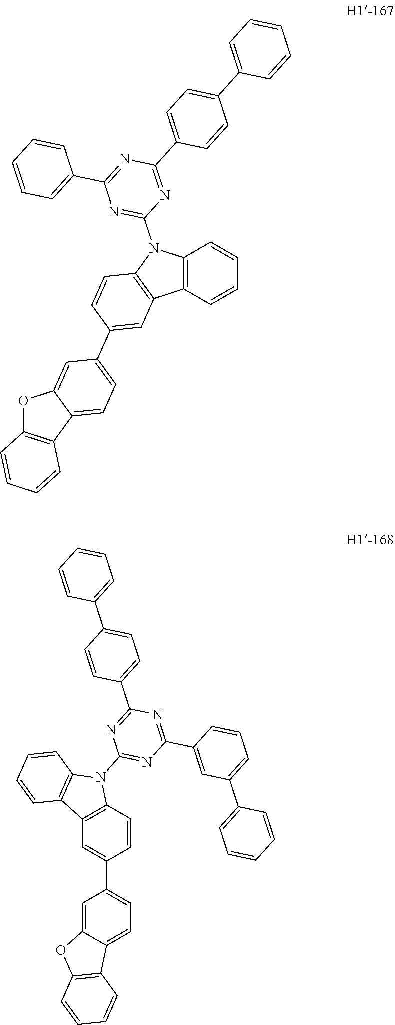 [SMILES: c1ccc(-c2ccc(-c3nc(-c4ccccc4)nc(-n4c5ccccc5c5cc(-c6ccc7c(c6)oc6ccccc67)ccc54)n3)cc2)cc1 ; c1ccc(-c2ccc(-c3nc(-c4cccc(-c5ccccc5)c4)nc(-n4c5ccccc5c5cc(-c6ccc7c(c6)oc6ccccc67)ccc54)n3)cc2)cc1]

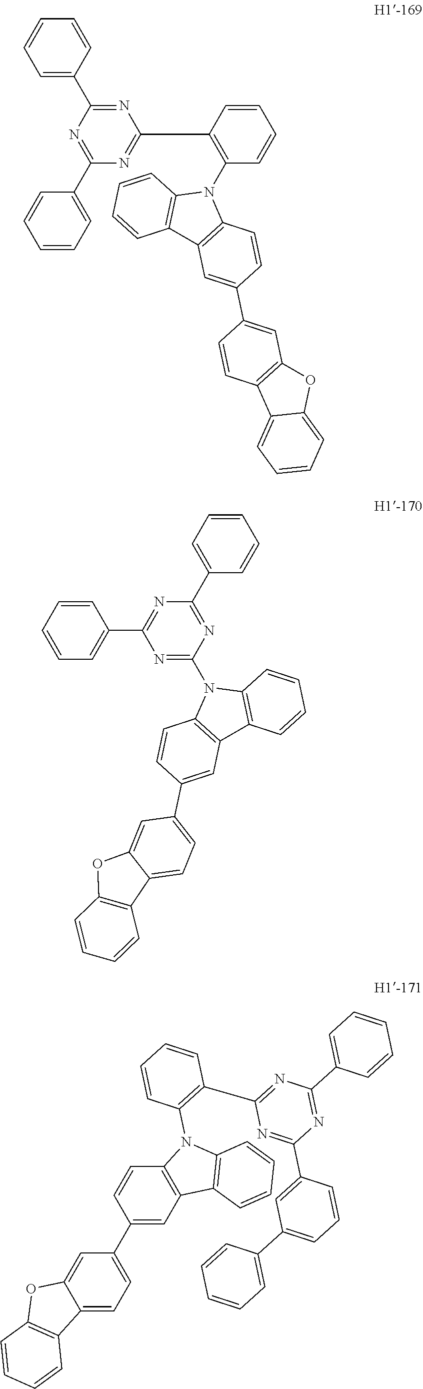 [SMILES: c1ccc(-c2nc(-c3ccccc3)nc(-c3ccccc3-n3c4ccccc4c4cc(-c5ccc6c(c5)oc5ccccc56)ccc43)n2)cc1 ; c1ccc(-c2nc(-c3ccccc3)nc(-n3c4ccccc4c4cc(-c5ccc6c(c5)oc5ccccc56)ccc43)n2)cc1 ; c1ccc(-c2cccc(-c3nc(-c4ccccc4)nc(-c4ccccc4-n4c5ccccc5c5cc(-c6ccc7c(c6)oc6ccccc67)ccc54)n3)c2)cc1]

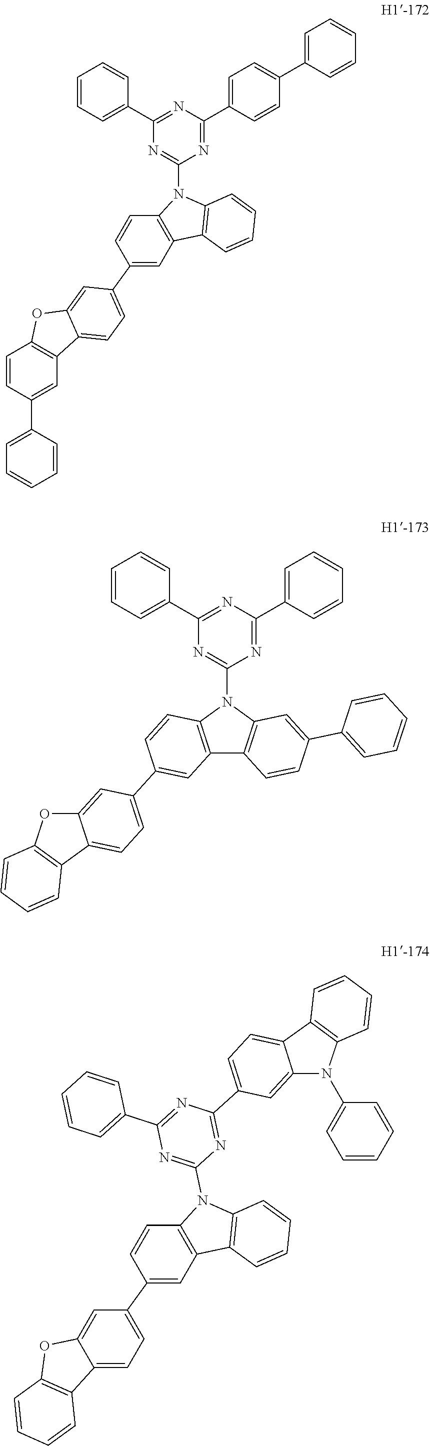 [SMILES: c1ccc(-c2ccc(-c3nc(-c4ccccc4)nc(-n4c5ccccc5c5cc(-c6ccc7c(c6)oc6ccc(-c8ccccc8)cc67)ccc54)n3)cc2)cc1 ; c1ccc(-c2ccc3c4cc(-c5ccc6c(c5)oc5ccccc56)ccc4n(-c4nc(-c5ccccc5)nc(-c5ccccc5)n4)c3c2)cc1 ; c1ccc(-c2nc(-c3ccc4c5ccccc5n(-c5ccccc5)c4c3)nc(-n3c4ccccc4c4cc(-c5ccc6c(c5)oc5ccccc56)ccc43)n2)cc1]

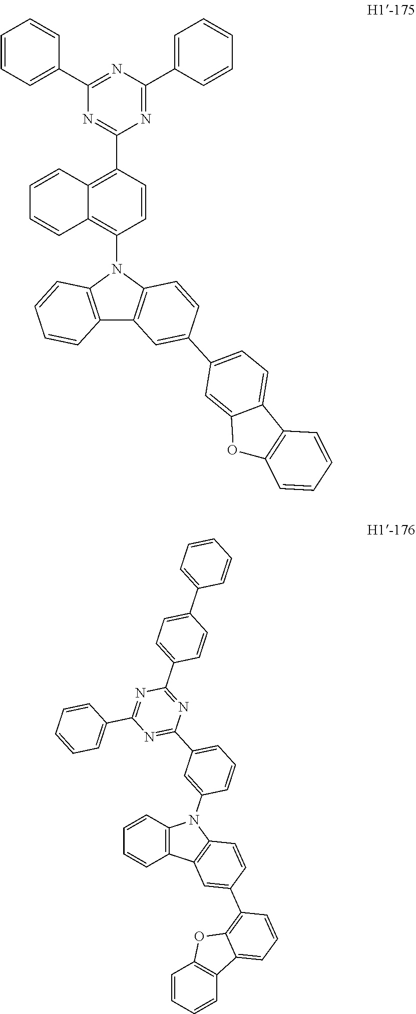 [SMILES: c1ccc(-c2nc(-c3ccccc3)nc(-c3ccc(-n4c5ccccc5c5cc(-c6ccc7c(c6)oc6ccccc67)ccc54)c4ccccc34)n2)cc1 ; c1ccc(-c2ccc(-c3nc(-c4ccccc4)nc(-c4cccc(-n5c6ccccc6c6cc(-c7cccc8c7oc7ccccc78)ccc65)c4)n3)cc2)cc1]

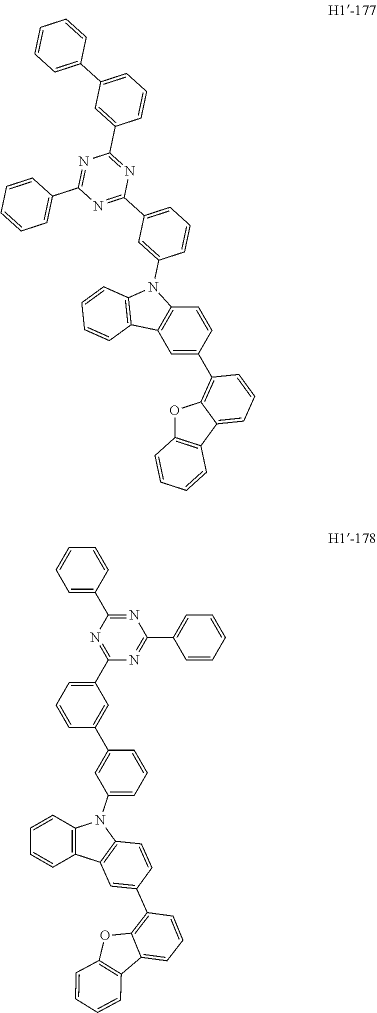 [SMILES: c1ccc(-c2cccc(-c3nc(-c4ccccc4)nc(-c4cccc(-n5c6ccccc6c6cc(-c7cccc8c7oc7ccccc78)ccc65)c4)n3)c2)cc1 ; c1ccc(-c2nc(-c3ccccc3)nc(-c3cccc(-c4cccc(-n5c6ccccc6c6cc(-c7cccc8c7oc7ccccc78)ccc65)c4)c3)n2)cc1]

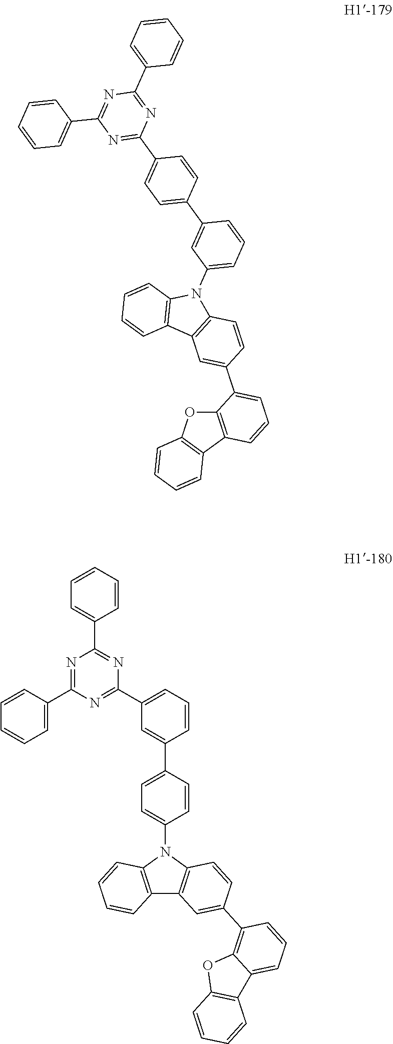 [SMILES: c1ccc(-c2nc(-c3ccccc3)nc(-c3ccc(-c4cccc(-n5c6ccccc6c6cc(-c7cccc8c7oc7ccccc78)ccc65)c4)cc3)n2)cc1 ; c1ccc(-c2nc(-c3ccccc3)nc(-c3cccc(-c4ccc(-n5c6ccccc6c6cc(-c7cccc8c7oc7ccccc78)ccc65)cc4)c3)n2)cc1]

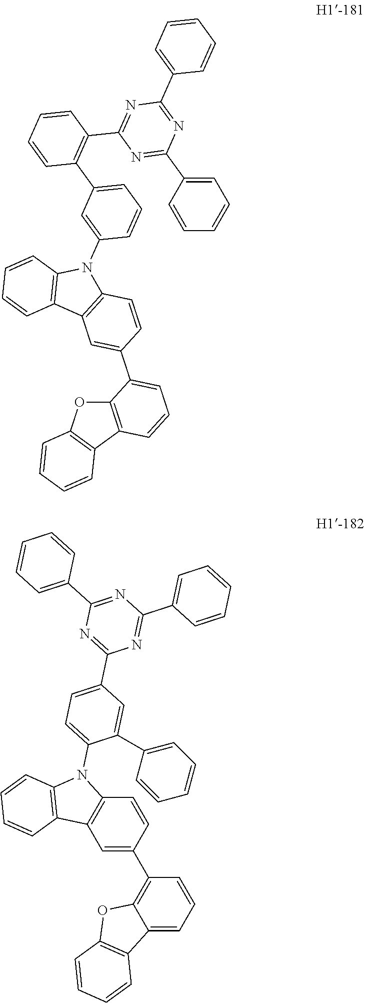 [SMILES: c1ccc(-c2nc(-c3ccccc3)nc(-c3ccccc3-c3cccc(-n4c5ccccc5c5cc(-c6cccc7c6oc6ccccc67)ccc54)c3)n2)cc1 ; c1ccc(-c2nc(-c3ccccc3)nc(-c3ccc(-n4c5ccccc5c5cc(-c6cccc7c6oc6ccccc67)ccc54)c(-c4ccccc4)c3)n2)cc1]

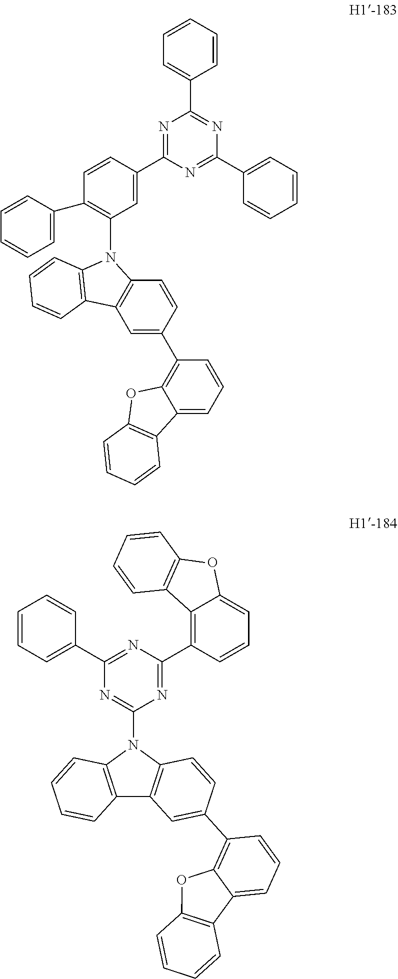 [SMILES: c1ccc(-c2nc(-c3ccccc3)nc(-c3ccc(-c4ccccc4)c(-n4c5ccccc5c5cc(-c6cccc7c6oc6ccccc67)ccc54)c3)n2)cc1 ; c1ccc(-c2nc(-c3cccc4oc5ccccc5c34)nc(-n3c4ccccc4c4cc(-c5cccc6c5oc5ccccc56)ccc43)n2)cc1]

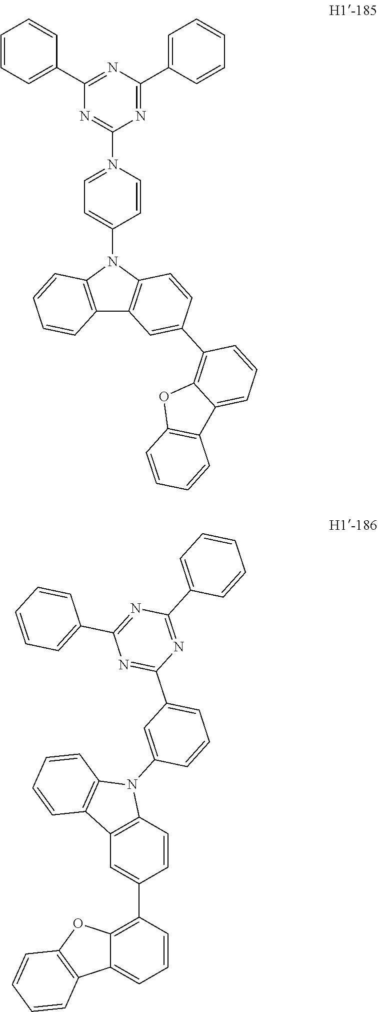

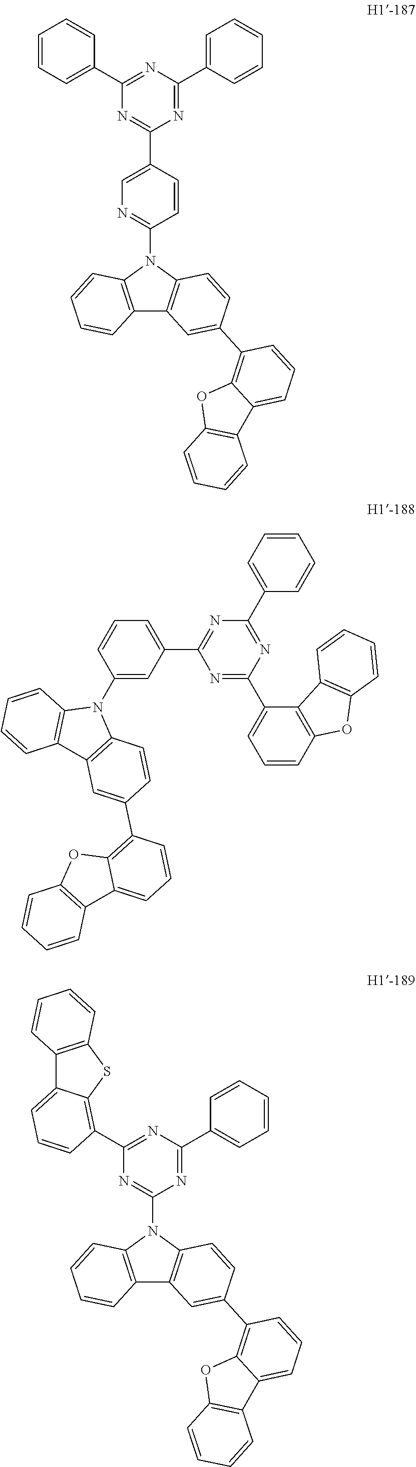

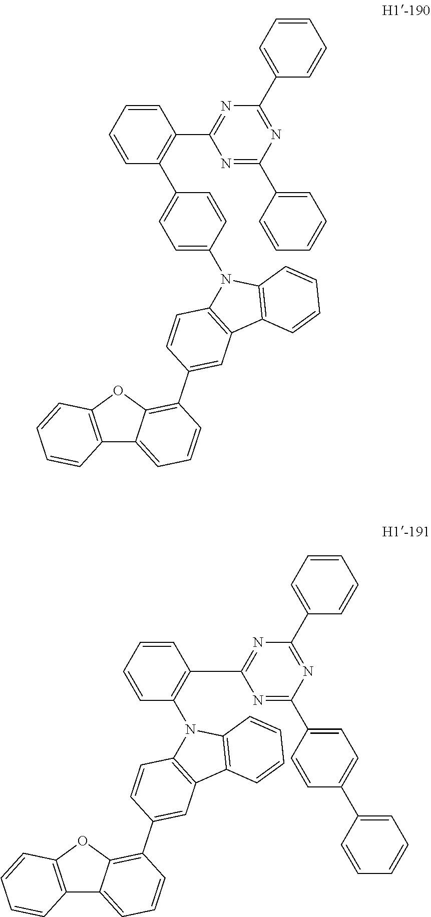 [SMILES: c1ccc(-c2nc(-c3ccccc3)nc(-c3ccccc3-c3ccc(-n4c5ccccc5c5cc(-c6cccc7c6oc6ccccc67)ccc54)cc3)n2)cc1 ; c1ccc(-c2ccc(-c3nc(-c4ccccc4)nc(-c4ccccc4-n4c5ccccc5c5cc(-c6cccc7c6oc6ccccc67)ccc54)n3)cc2)cc1]

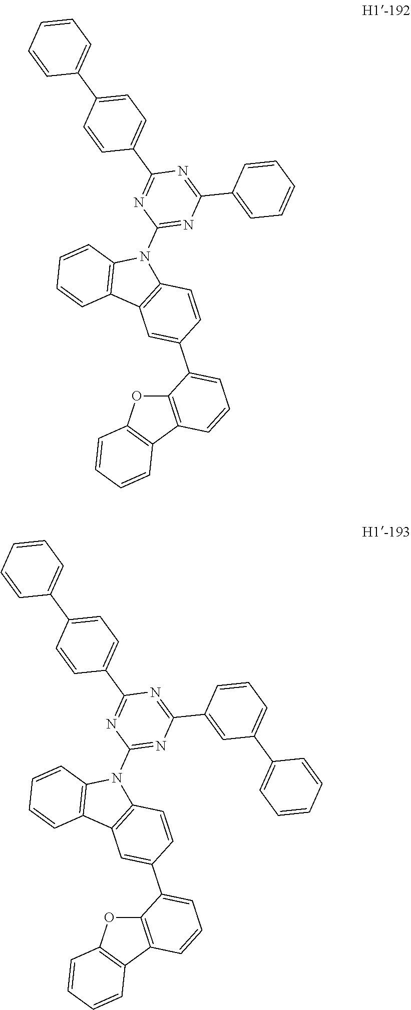 [SMILES: c1ccc(-c2ccc(-c3nc(-c4ccccc4)nc(-n4c5ccccc5c5cc(-c6cccc7c6oc6ccccc67)ccc54)n3)cc2)cc1 ; c1ccc(-c2ccc(-c3nc(-c4cccc(-c5ccccc5)c4)nc(-n4c5ccccc5c5cc(-c6cccc7c6oc6ccccc67)ccc54)n3)cc2)cc1]

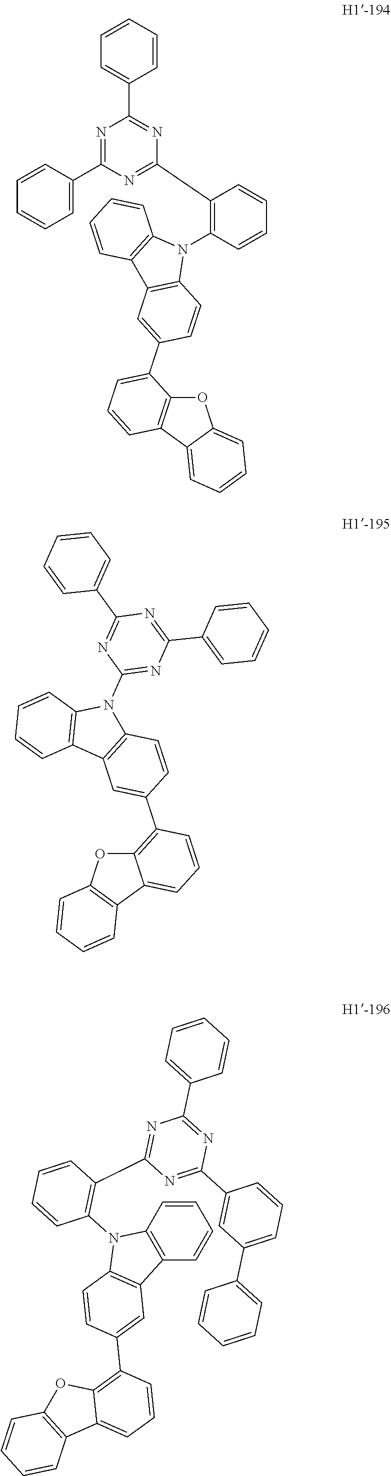 [SMILES: c1ccc(-c2nc(-c3ccccc3)nc(-c3ccccc3-n3c4ccccc4c4cc(-c5cccc6c5oc5ccccc56)ccc43)n2)cc1 ; c1ccc(-c2nc(-c3ccccc3)nc(-n3c4ccccc4c4cc(-c5cccc6c5oc5ccccc56)ccc43)n2)cc1 ; c1ccc(-c2cccc(-c3nc(-c4ccccc4)nc(-c4ccccc4-n4c5ccccc5c5cc(-c6cccc7c6oc6ccccc67)ccc54)n3)c2)cc1]

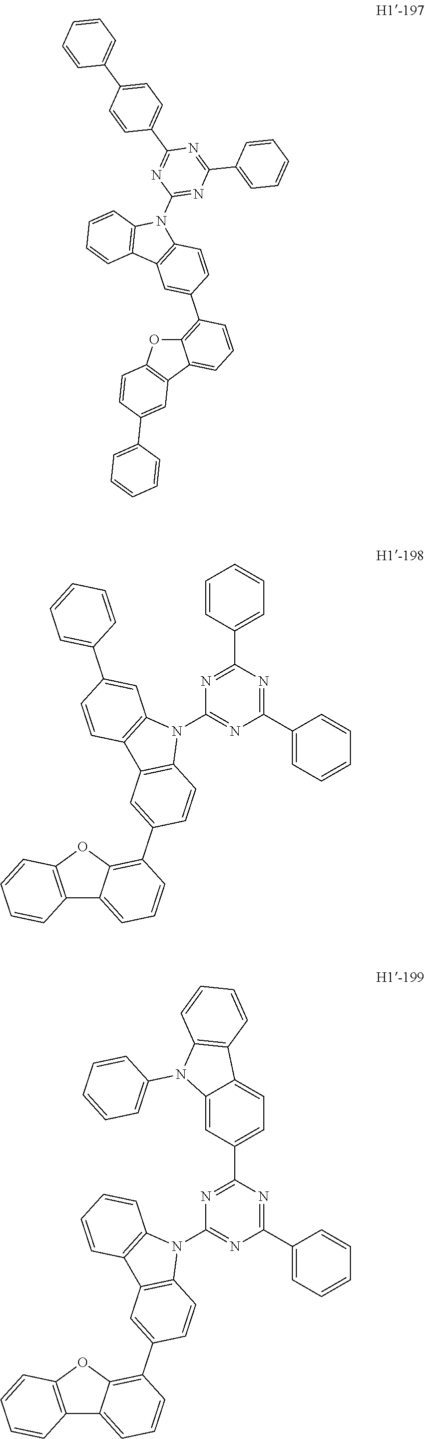 [SMILES: c1ccc(-c2ccc(-c3nc(-c4ccccc4)nc(-n4c5ccccc5c5cc(-c6cccc7c6oc6ccc(-c8ccccc8)cc67)ccc54)n3)cc2)cc1 ; c1ccc(-c2ccc3c4cc(-c5cccc6c5oc5ccccc56)ccc4n(-c4nc(-c5ccccc5)nc(-c5ccccc5)n4)c3c2)cc1 ; c1ccc(-c2nc(-c3ccc4c5ccccc5n(-c5ccccc5)c4c3)nc(-n3c4ccccc4c4cc(-c5cccc6c5oc5ccccc56)ccc43)n2)cc1]

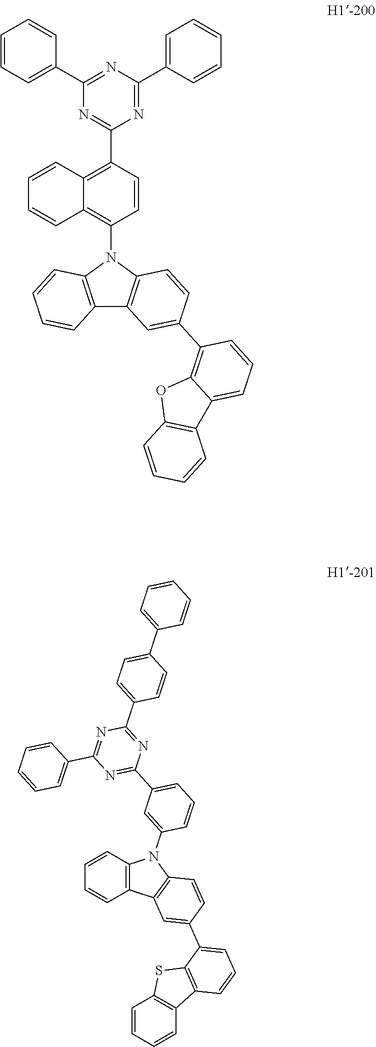 [SMILES: c1ccc(-c2nc(-c3ccccc3)nc(-c3ccc(-n4c5ccccc5c5cc(-c6cccc7c6oc6ccccc67)ccc54)c4ccccc34)n2)cc1 ; c1ccc(-c2ccc(-c3nc(-c4ccccc4)nc(-c4cccc(-n5c6ccccc6c6cc(-c7cccc8c7sc7ccccc78)ccc65)c4)n3)cc2)cc1]

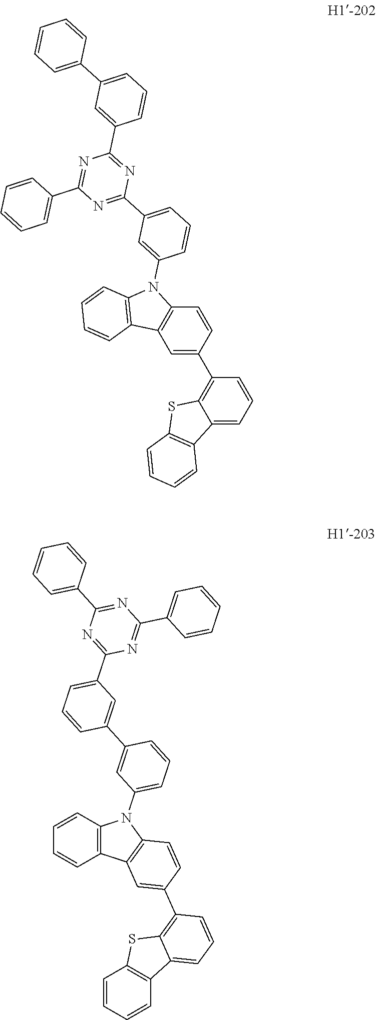 [SMILES: c1ccc(-c2cccc(-c3nc(-c4ccccc4)nc(-c4cccc(-n5c6ccccc6c6cc(-c7cccc8c7sc7ccccc78)ccc65)c4)n3)c2)cc1 ; c1ccc(-c2nc(-c3ccccc3)nc(-c3cccc(-c4cccc(-n5c6ccccc6c6cc(-c7cccc8c7sc7ccccc78)ccc65)c4)c3)n2)cc1]

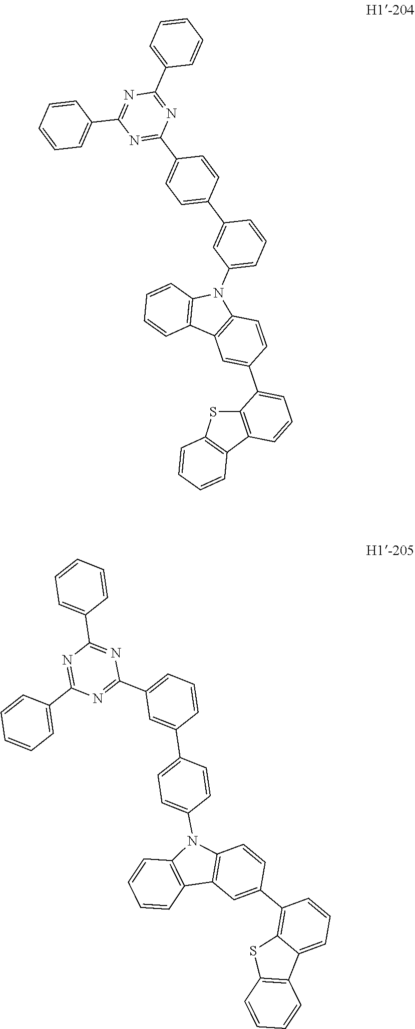 [SMILES: c1ccc(-c2nc(-c3ccccc3)nc(-c3ccc(-c4cccc(-n5c6ccccc6c6cc(-c7cccc8c7sc7ccccc78)ccc65)c4)cc3)n2)cc1 ; c1ccc(-c2nc(-c3ccccc3)nc(-c3cccc(-c4ccc(-n5c6ccccc6c6cc(-c7cccc8c7sc7ccccc78)ccc65)cc4)c3)n2)cc1]

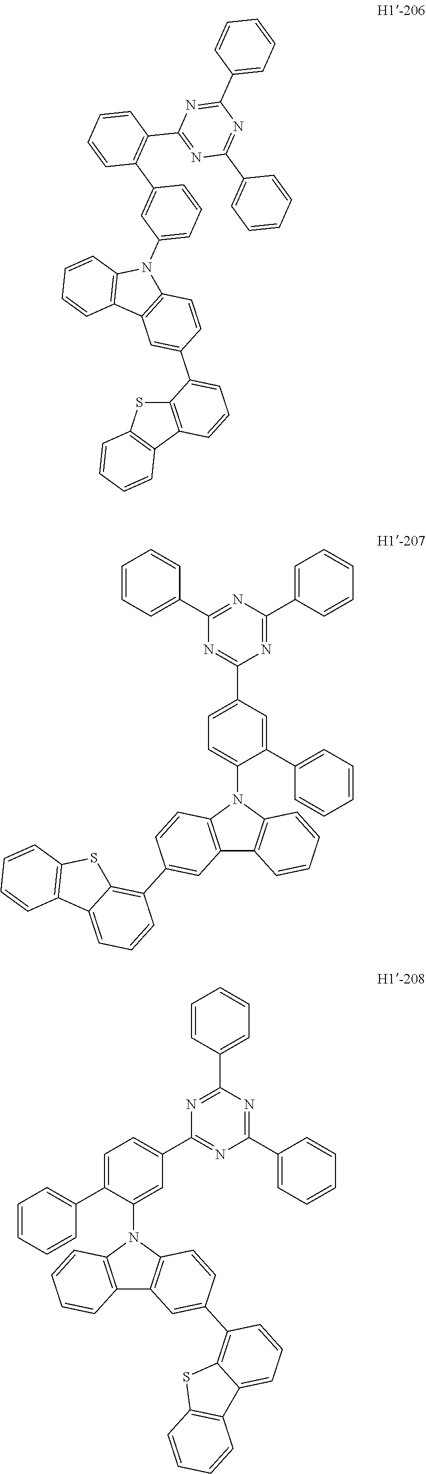 [SMILES: c1ccc(-c2nc(-c3ccccc3)nc(-c3ccccc3-c3cccc(-n4c5ccccc5c5cc(-c6cccc7c6sc6ccccc67)ccc54)c3)n2)cc1 ; c1ccc(-c2nc(-c3ccccc3)nc(-c3ccc(-n4c5ccccc5c5cc(-c6cccc7c6sc6ccccc67)ccc54)c(-c4ccccc4)c3)n2)cc1 ; c1ccc(-c2nc(-c3ccccc3)nc(-c3ccc(-c4ccccc4)c(-n4c5ccccc5c5cc(-c6cccc7c6sc6ccccc67)ccc54)c3)n2)cc1]

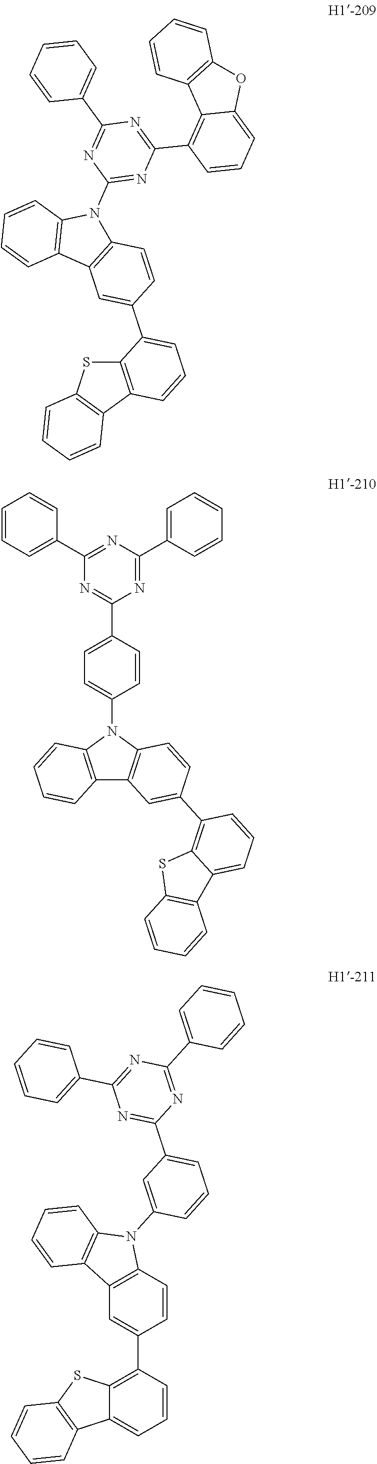

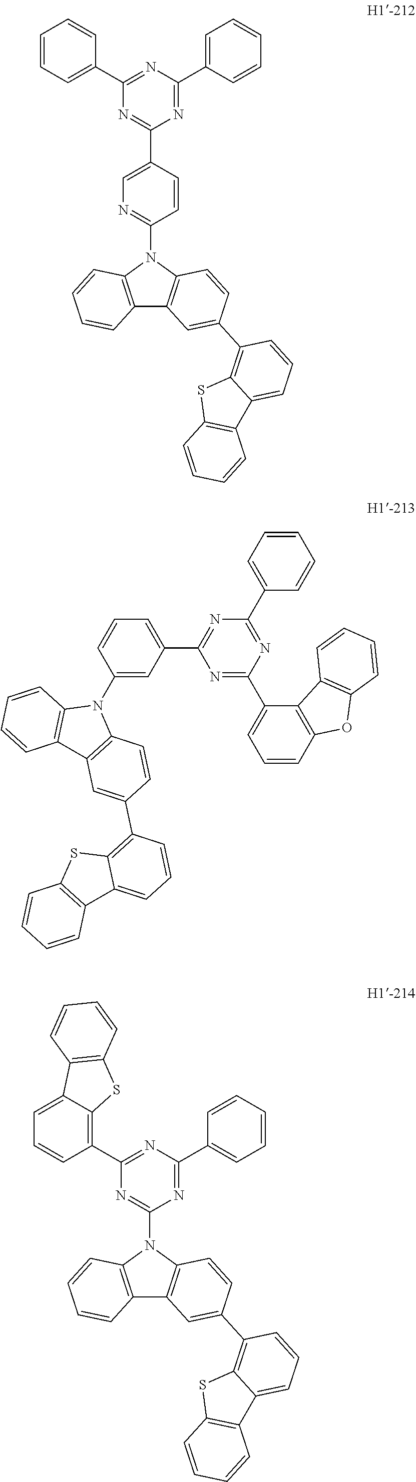 [SMILES: c1ccc(-c2nc(-c3ccccc3)nc(-c3ccc(-n4c5ccccc5c5cc(-c6cccc7c6sc6ccccc67)ccc54)nc3)n2)cc1 ; c1ccc(-c2nc(-c3cccc(-n4c5ccccc5c5cc(-c6cccc7c6sc6ccccc67)ccc54)c3)nc(-c3cccc4oc5ccccc5c34)n2)cc1 ; c1ccc(-c2nc(-c3cccc4c3sc3ccccc34)nc(-n3c4ccccc4c4cc(-c5cccc6c5sc5ccccc56)ccc43)n2)cc1]

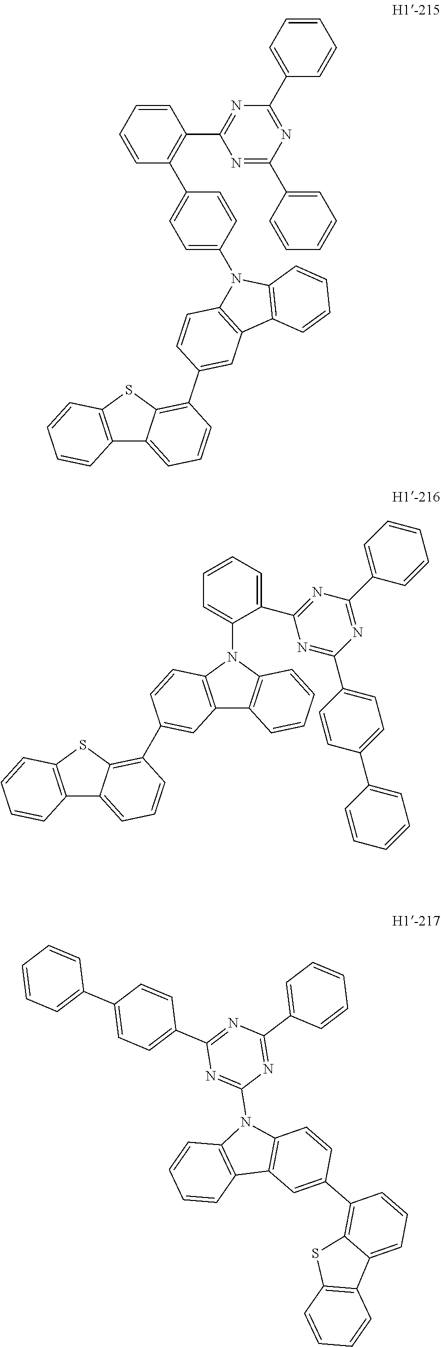 [SMILES: c1ccc(-c2nc(-c3ccccc3)nc(-c3ccccc3-c3ccc(-n4c5ccccc5c5cc(-c6cccc7c6sc6ccccc67)ccc54)cc3)n2)cc1 ; c1ccc(-c2ccc(-c3nc(-c4ccccc4)nc(-c4ccccc4-n4c5ccccc5c5cc(-c6cccc7c6sc6ccccc67)ccc54)n3)cc2)cc1 ; c1ccc(-c2ccc(-c3nc(-c4ccccc4)nc(-n4c5ccccc5c5cc(-c6cccc7c6sc6ccccc67)ccc54)n3)cc2)cc1]

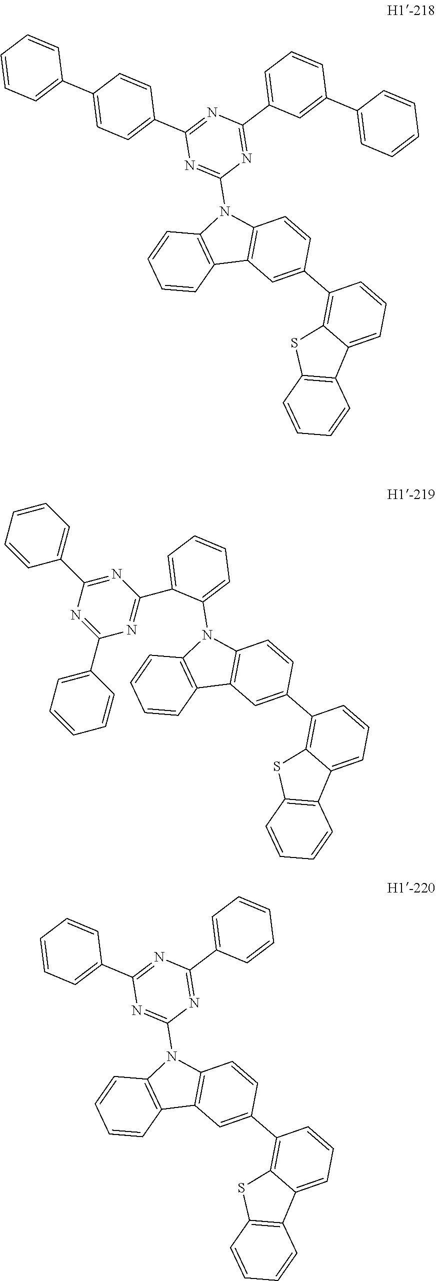 [SMILES: c1ccc(-c2ccc(-c3nc(-c4cccc(-c5ccccc5)c4)nc(-n4c5ccccc5c5cc(-c6cccc7c6sc6ccccc67)ccc54)n3)cc2)cc1 ; c1ccc(-c2nc(-c3ccccc3)nc(-c3ccccc3-n3c4ccccc4c4cc(-c5cccc6c5sc5ccccc56)ccc43)n2)cc1 ; c1ccc(-c2nc(-c3ccccc3)nc(-n3c4ccccc4c4cc(-c5cccc6c5sc5ccccc56)ccc43)n2)cc1]

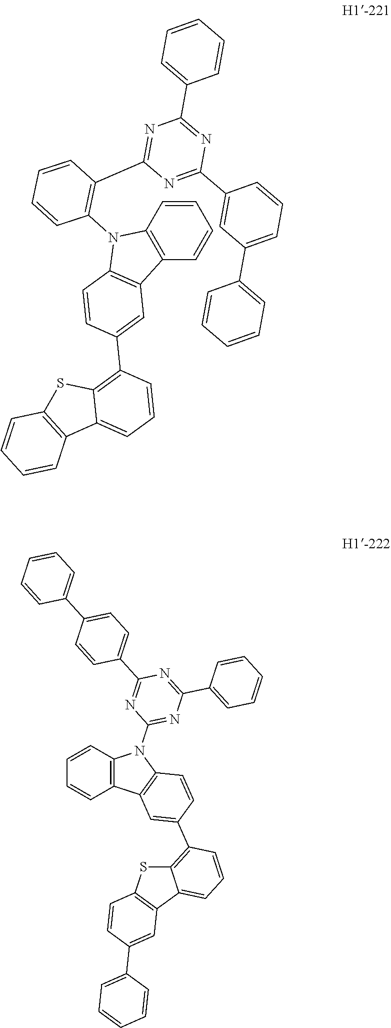 [SMILES: c1ccc(-c2cccc(-c3nc(-c4ccccc4)nc(-c4ccccc4-n4c5ccccc5c5cc(-c6cccc7c6sc6ccccc67)ccc54)n3)c2)cc1 ; c1ccc(-c2ccc(-c3nc(-c4ccccc4)nc(-n4c5ccccc5c5cc(-c6cccc7c6sc6ccc(-c8ccccc8)cc67)ccc54)n3)cc2)cc1]

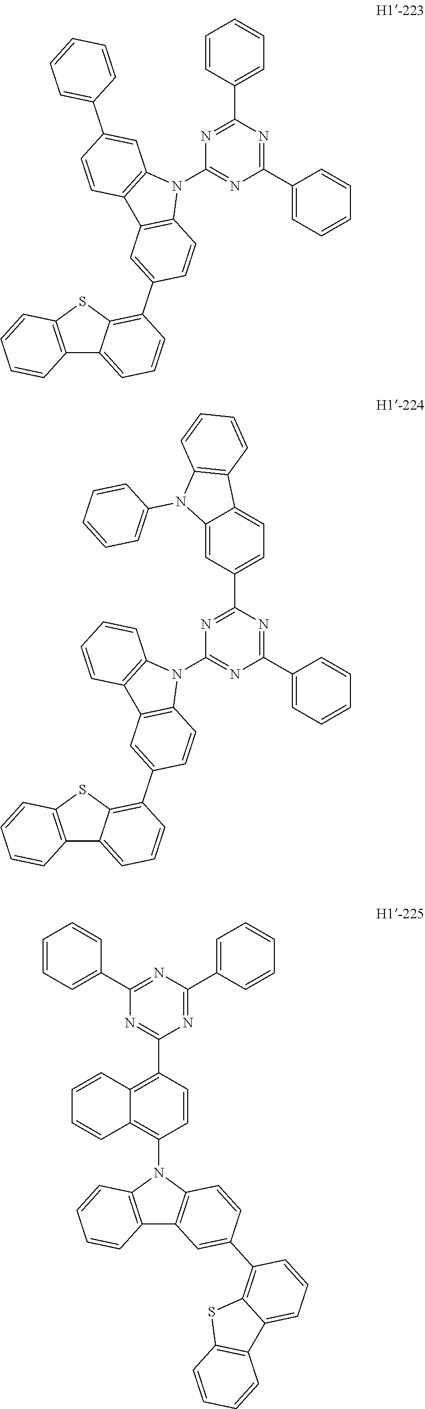 [SMILES: c1ccc(-c2ccc3c4cc(-c5cccc6c5sc5ccccc56)ccc4n(-c4nc(-c5ccccc5)nc(-c5ccccc5)n4)c3c2)cc1 ; c1ccc(-c2nc(-c3ccc4c5ccccc5n(-c5ccccc5)c4c3)nc(-n3c4ccccc4c4cc(-c5cccc6c5sc5ccccc56)ccc43)n2)cc1 ; c1ccc(-c2nc(-c3ccccc3)nc(-c3ccc(-n4c5ccccc5c5cc(-c6cccc7c6sc6ccccc67)ccc54)c4ccccc34)n2)cc1]

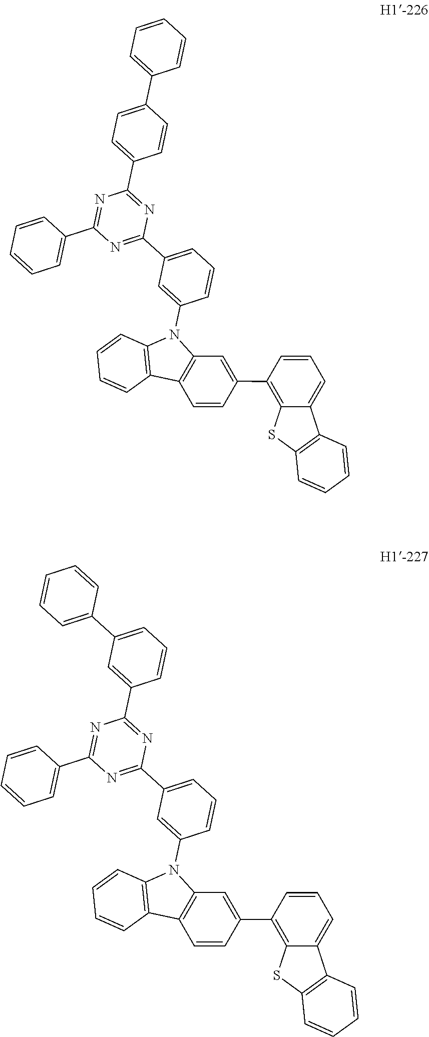 [SMILES: c1ccc(-c2ccc(-c3nc(-c4ccccc4)nc(-c4cccc(-n5c6ccccc6c6ccc(-c7cccc8c7sc7ccccc78)cc65)c4)n3)cc2)cc1 ; c1ccc(-c2cccc(-c3nc(-c4ccccc4)nc(-c4cccc(-n5c6ccccc6c6ccc(-c7cccc8c7sc7ccccc78)cc65)c4)n3)c2)cc1]

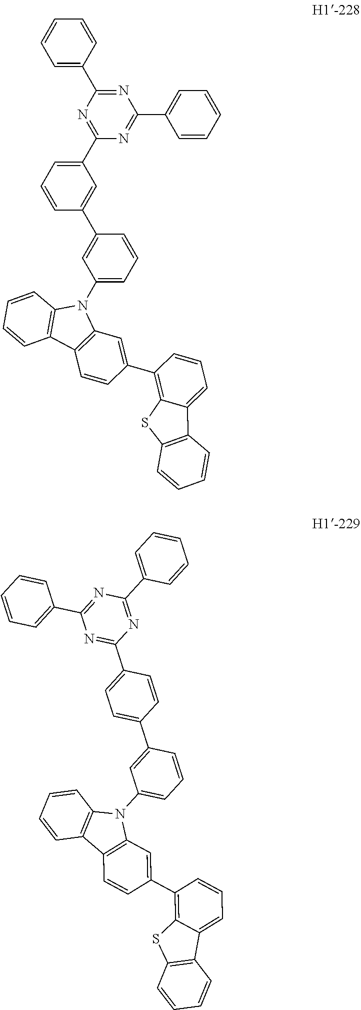 [SMILES: c1ccc(-c2nc(-c3ccccc3)nc(-c3cccc(-c4cccc(-n5c6ccccc6c6ccc(-c7cccc8c7sc7ccccc78)cc65)c4)c3)n2)cc1 ; c1ccc(-c2nc(-c3ccccc3)nc(-c3ccc(-c4cccc(-n5c6ccccc6c6ccc(-c7cccc8c7sc7ccccc78)cc65)c4)cc3)n2)cc1]

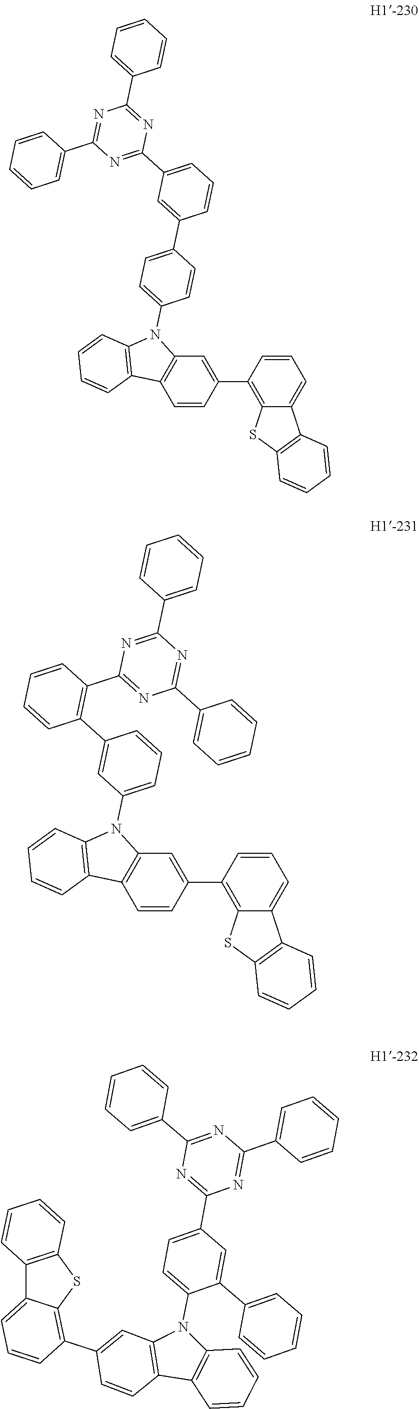 [SMILES: c1ccc(-c2nc(-c3ccccc3)nc(-c3cccc(-c4ccc(-n5c6ccccc6c6ccc(-c7cccc8c7sc7ccccc78)cc65)cc4)c3)n2)cc1 ; c1ccc(-c2nc(-c3ccccc3)nc(-c3ccccc3-c3cccc(-n4c5ccccc5c5ccc(-c6cccc7c6sc6ccccc67)cc54)c3)n2)cc1 ; c1ccc(-c2nc(-c3ccccc3)nc(-c3ccc(-n4c5ccccc5c5ccc(-c6cccc7c6sc6ccccc67)cc54)c(-c4ccccc4)c3)n2)cc1]

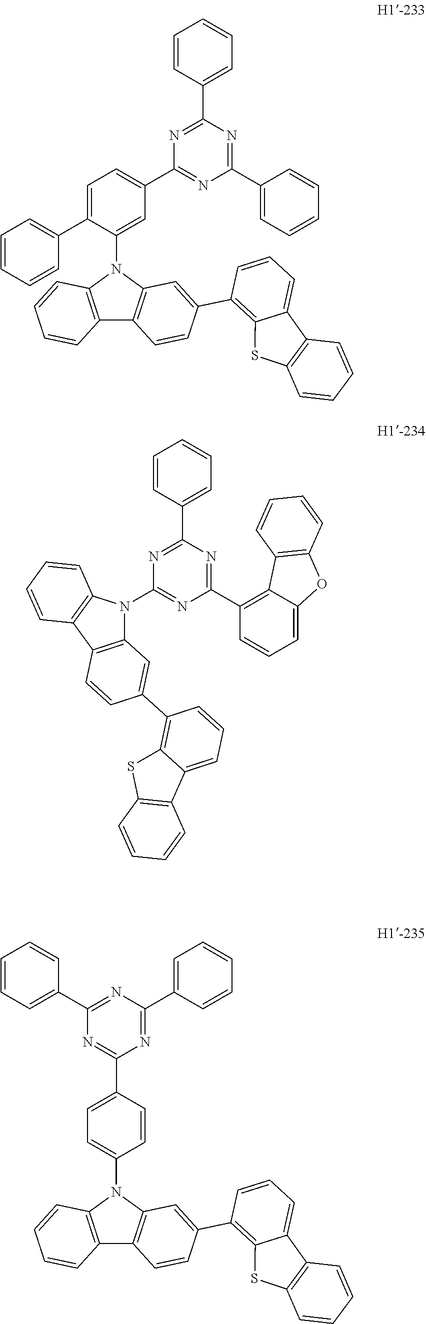 [SMILES: c1ccc(-c2nc(-c3ccccc3)nc(-c3ccc(-c4ccccc4)c(-n4c5ccccc5c5ccc(-c6cccc7c6sc6ccccc67)cc54)c3)n2)cc1 ; c1ccc(-c2nc(-c3cccc4oc5ccccc5c34)nc(-n3c4ccccc4c4ccc(-c5cccc6c5sc5ccccc56)cc43)n2)cc1 ; c1ccc(-c2nc(-c3ccccc3)nc(-c3ccc(-n4c5ccccc5c5ccc(-c6cccc7c6sc6ccccc67)cc54)cc3)n2)cc1]

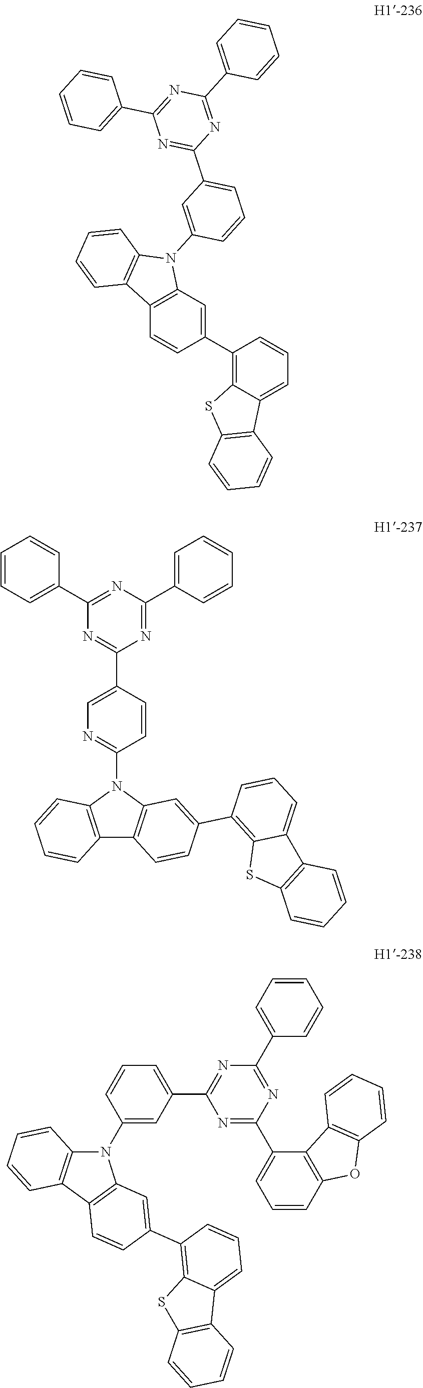 [SMILES: c1ccc(-c2nc(-c3ccccc3)nc(-c3cccc(-n4c5ccccc5c5ccc(-c6cccc7c6sc6ccccc67)cc54)c3)n2)cc1 ; c1ccc(-c2nc(-c3ccccc3)nc(-c3ccc(-n4c5ccccc5c5ccc(-c6cccc7c6sc6ccccc67)cc54)nc3)n2)cc1 ; c1ccc(-c2nc(-c3cccc(-n4c5ccccc5c5ccc(-c6cccc7c6sc6ccccc67)cc54)c3)nc(-c3cccc4oc5ccccc5c34)n2)cc1]

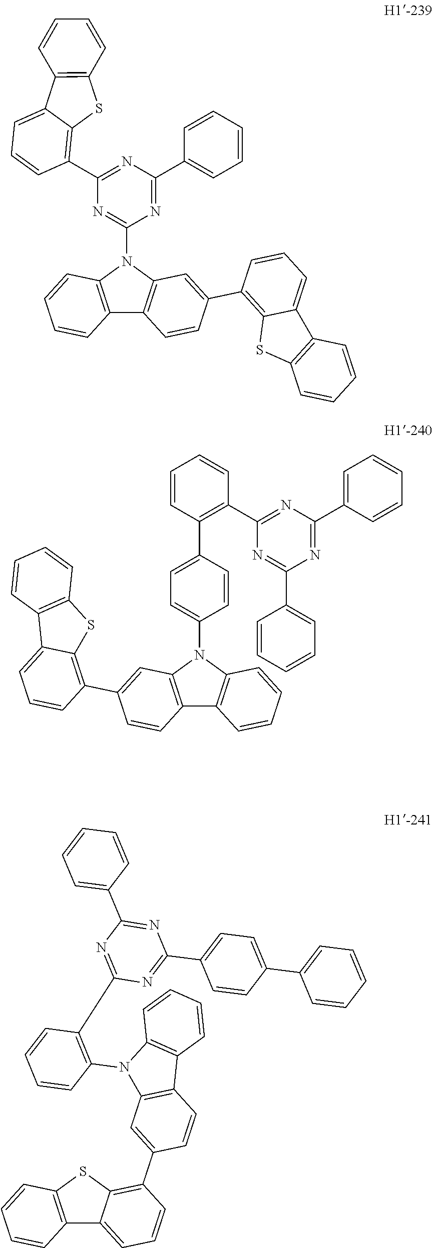 [SMILES: c1ccc(-c2nc(-c3cccc4c3sc3ccccc34)nc(-n3c4ccccc4c4ccc(-c5cccc6c5sc5ccccc56)cc43)n2)cc1 ; c1ccc(-c2nc(-c3ccccc3)nc(-c3ccccc3-c3ccc(-n4c5ccccc5c5ccc(-c6cccc7c6sc6ccccc67)cc54)cc3)n2)cc1 ; c1ccc(-c2ccc(-c3nc(-c4ccccc4)nc(-c4ccccc4-n4c5ccccc5c5ccc(-c6cccc7c6sc6ccccc67)cc54)n3)cc2)cc1]

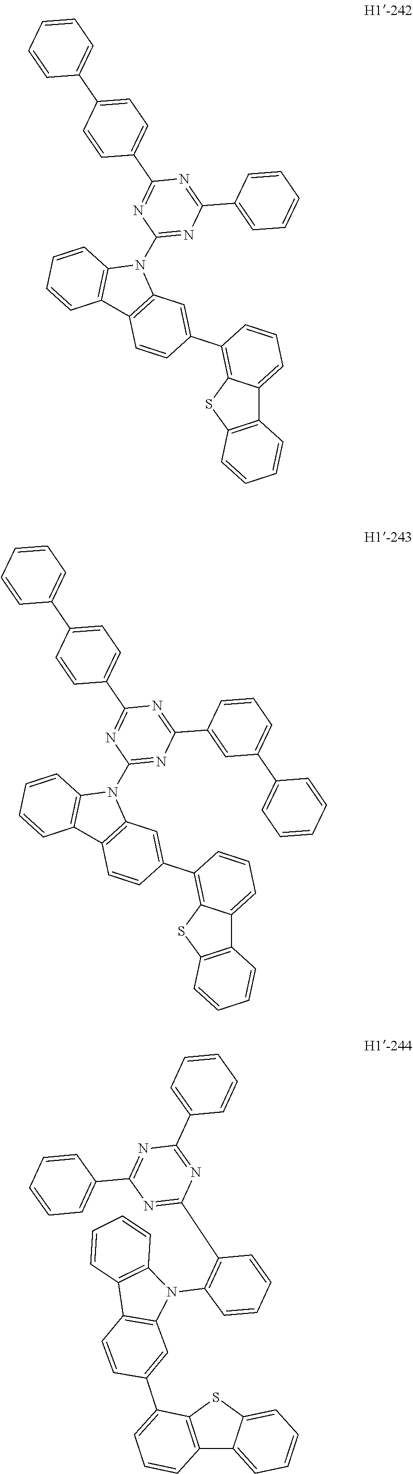

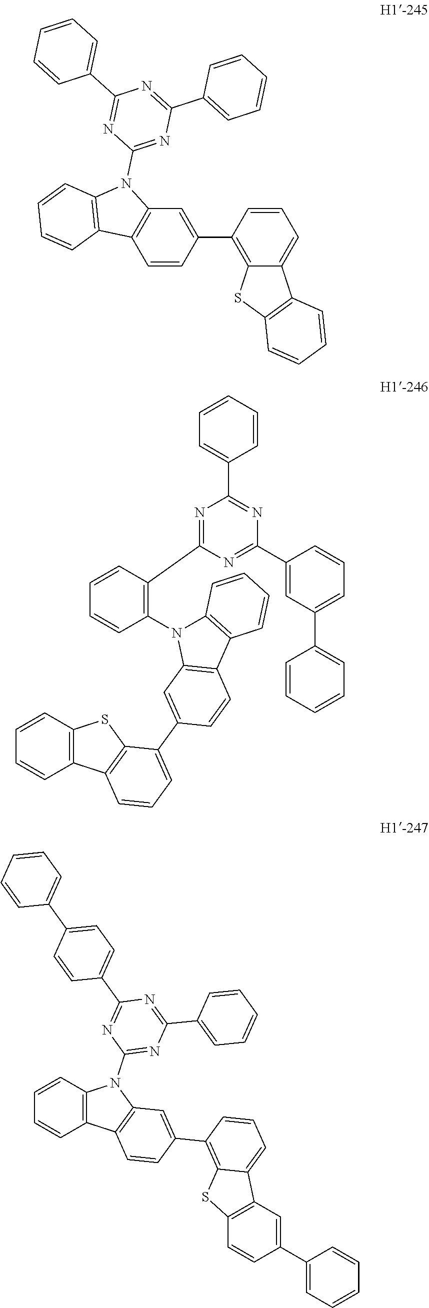 [SMILES: c1ccc(-c2nc(-c3ccccc3)nc(-n3c4ccccc4c4ccc(-c5cccc6c5sc5ccccc56)cc43)n2)cc1 ; c1ccc(-c2cccc(-c3nc(-c4ccccc4)nc(-c4ccccc4-n4c5ccccc5c5ccc(-c6cccc7c6sc6ccccc67)cc54)n3)c2)cc1 ; c1ccc(-c2ccc(-c3nc(-c4ccccc4)nc(-n4c5ccccc5c5ccc(-c6cccc7c6sc6ccc(-c8ccccc8)cc67)cc54)n3)cc2)cc1]

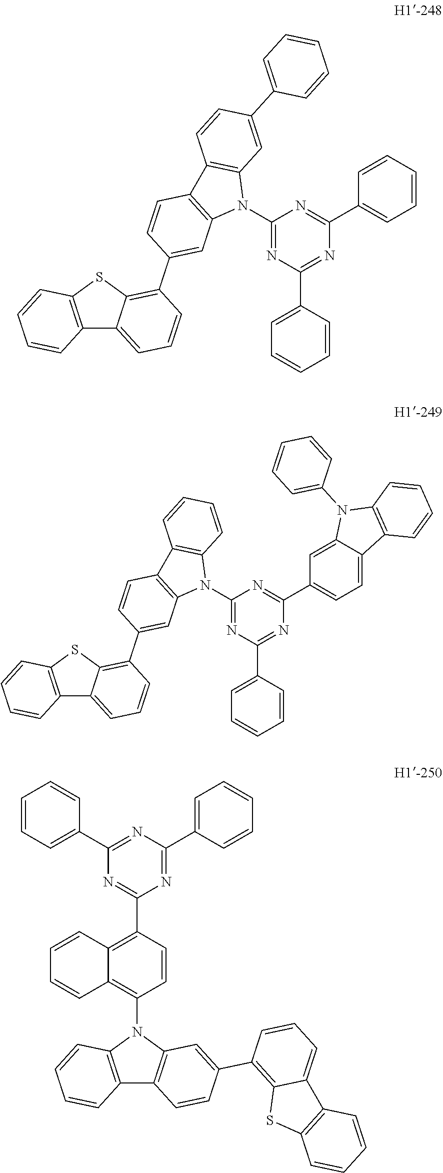 [SMILES: c1ccc(-c2ccc3c4ccc(-c5cccc6c5sc5ccccc56)cc4n(-c4nc(-c5ccccc5)nc(-c5ccccc5)n4)c3c2)cc1 ; c1ccc(-c2nc(-c3ccc4c5ccccc5n(-c5ccccc5)c4c3)nc(-n3c4ccccc4c4ccc(-c5cccc6c5sc5ccccc56)cc43)n2)cc1 ; c1ccc(-c2nc(-c3ccccc3)nc(-c3ccc(-n4c5ccccc5c5ccc(-c6cccc7c6sc6ccccc67)cc54)c4ccccc34)n2)cc1]

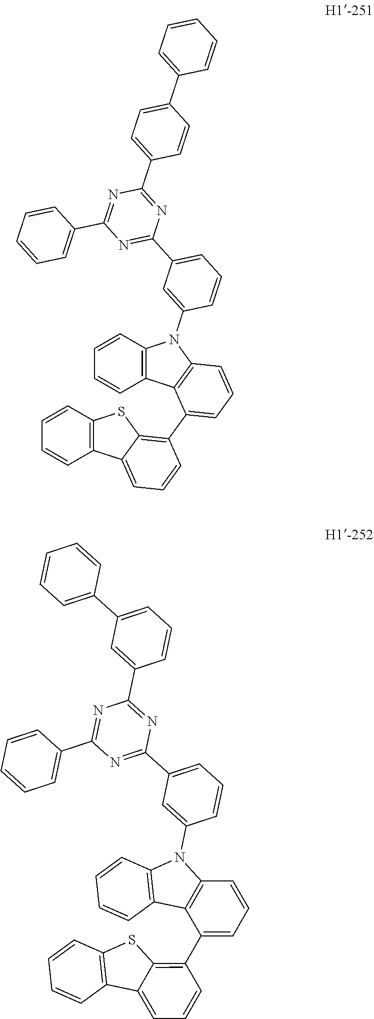 [SMILES: c1ccc(-c2ccc(-c3nc(-c4ccccc4)nc(-c4cccc(-n5c6ccccc6c6c(-c7cccc8c7sc7ccccc78)cccc65)c4)n3)cc2)cc1 ; c1ccc(-c2cccc(-c3nc(-c4ccccc4)nc(-c4cccc(-n5c6ccccc6c6c(-c7cccc8c7sc7ccccc78)cccc65)c4)n3)c2)cc1]

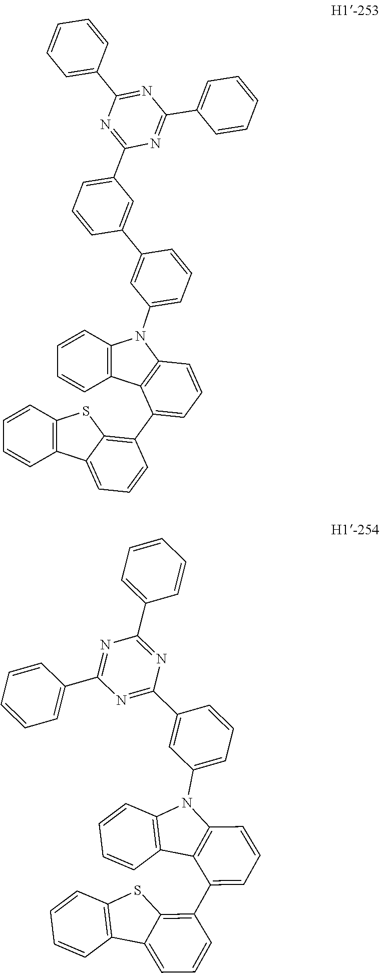 [SMILES: c1ccc(-c2nc(-c3ccccc3)nc(-c3cccc(-c4cccc(-n5c6ccccc6c6c(-c7cccc8c7sc7ccccc78)cccc65)c4)c3)n2)cc1 ; c1ccc(-c2nc(-c3ccccc3)nc(-c3cccc(-n4c5ccccc5c5c(-c6cccc7c6sc6ccccc67)cccc54)c3)n2)cc1]

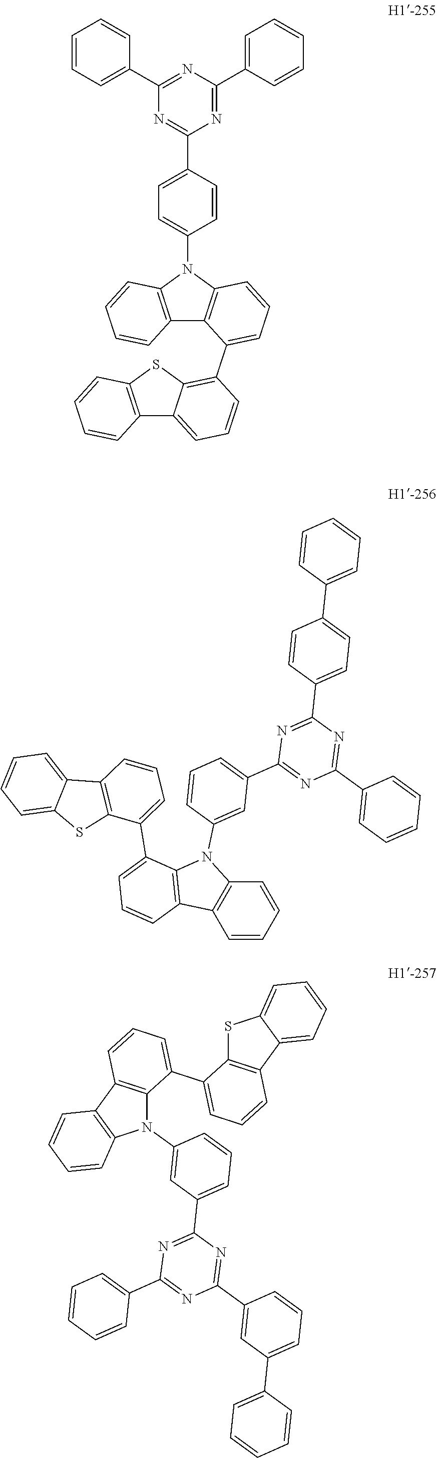 [SMILES: c1ccc(-c2nc(-c3ccccc3)nc(-c3ccc(-n4c5ccccc5c5c(-c6cccc7c6sc6ccccc67)cccc54)cc3)n2)cc1 ; c1ccc(-c2ccc(-c3nc(-c4ccccc4)nc(-c4cccc(-n5c6ccccc6c6cccc(-c7cccc8c7sc7ccccc78)c65)c4)n3)cc2)cc1 ; c1ccc(-c2cccc(-c3nc(-c4ccccc4)nc(-c4cccc(-n5c6ccccc6c6cccc(-c7cccc8c7sc7ccccc78)c65)c4)n3)c2)cc1]

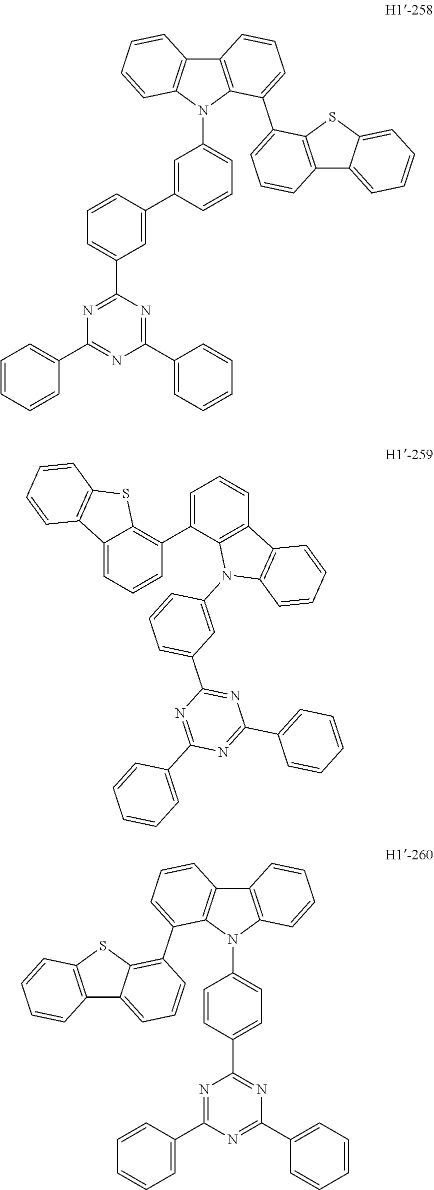

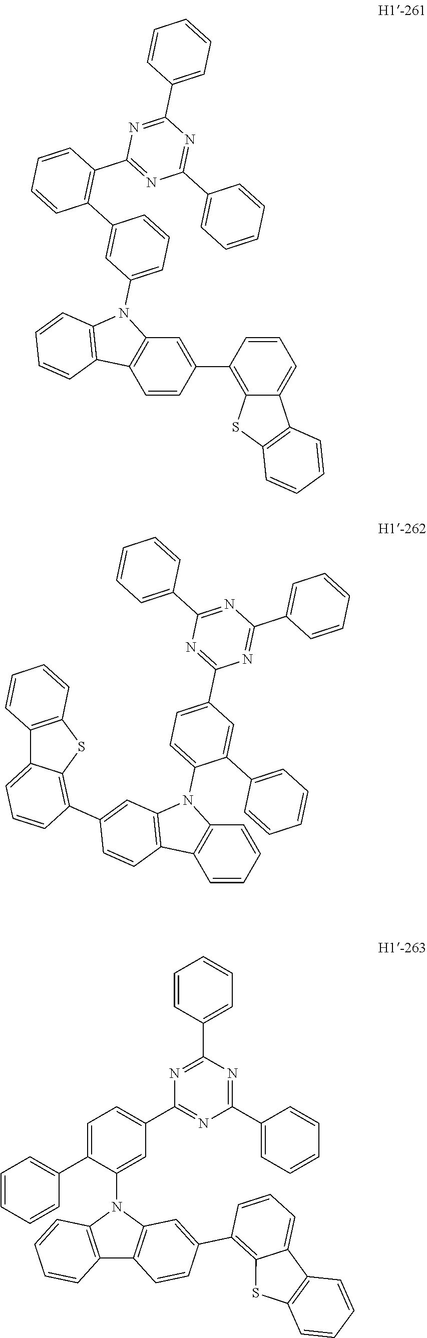 [SMILES: c1ccc(-c2nc(-c3ccccc3)nc(-c3ccccc3-c3cccc(-n4c5ccccc5c5ccc(-c6cccc7c6sc6ccccc67)cc54)c3)n2)cc1 ; c1ccc(-c2nc(-c3ccccc3)nc(-c3ccc(-n4c5ccccc5c5ccc(-c6cccc7c6sc6ccccc67)cc54)c(-c4ccccc4)c3)n2)cc1 ; c1ccc(-c2nc(-c3ccccc3)nc(-c3ccc(-c4ccccc4)c(-n4c5ccccc5c5ccc(-c6cccc7c6sc6ccccc67)cc54)c3)n2)cc1]

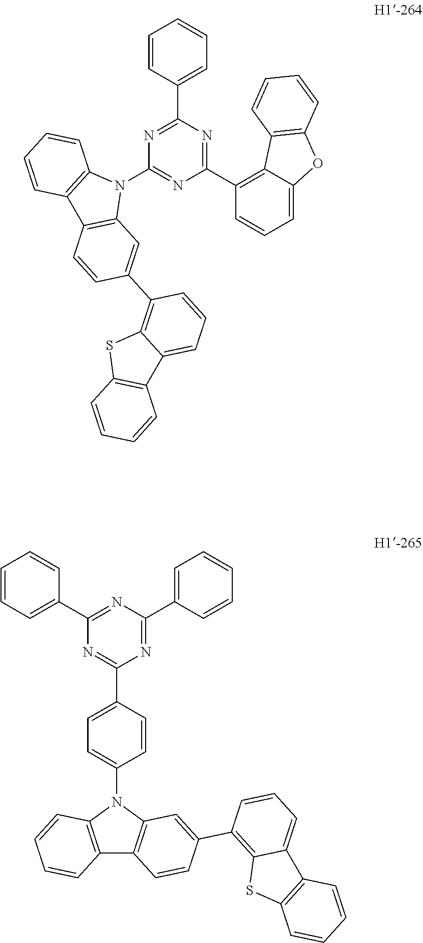 [SMILES: c1ccc(-c2nc(-c3cccc4oc5ccccc5c34)nc(-n3c4ccccc4c4ccc(-c5cccc6c5sc5ccccc56)cc43)n2)cc1 ; c1ccc(-c2nc(-c3ccccc3)nc(-c3ccc(-n4c5ccccc5c5ccc(-c6cccc7c6sc6ccccc67)cc54)cc3)n2)cc1]

The compound represented by formula 2 may be at least one selected from the following compounds, but is not limited thereto.

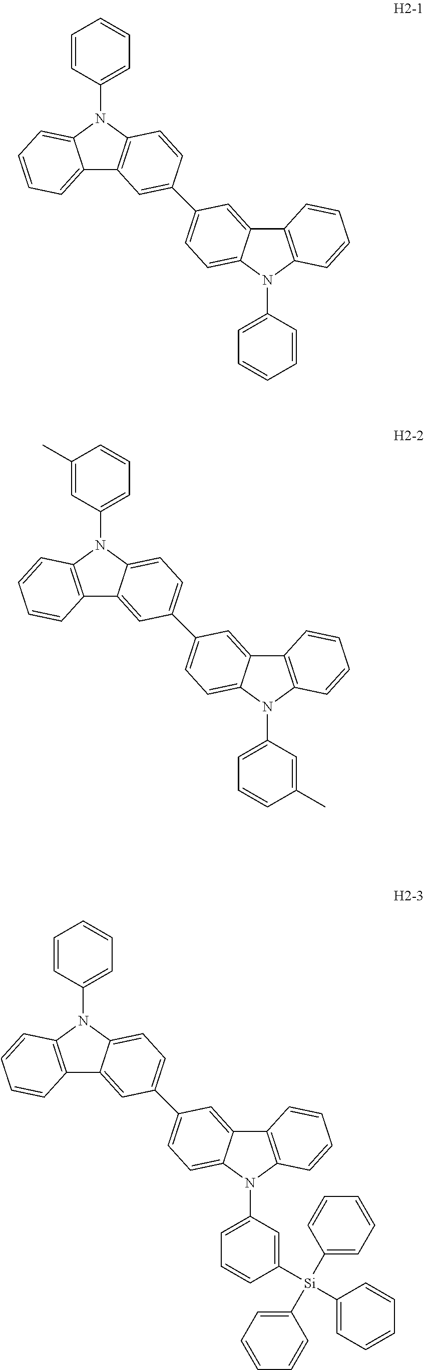 [SMILES: c1ccc(-n2c3ccccc3c3cc(-c4ccc5c(c4)c4ccccc4n5-c4ccccc4)ccc32)cc1 ; Cc1cccc(-n2c3ccccc3c3cc(-c4ccc5c(c4)c4ccccc4n5-c4cccc(C)c4)ccc32)c1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1]

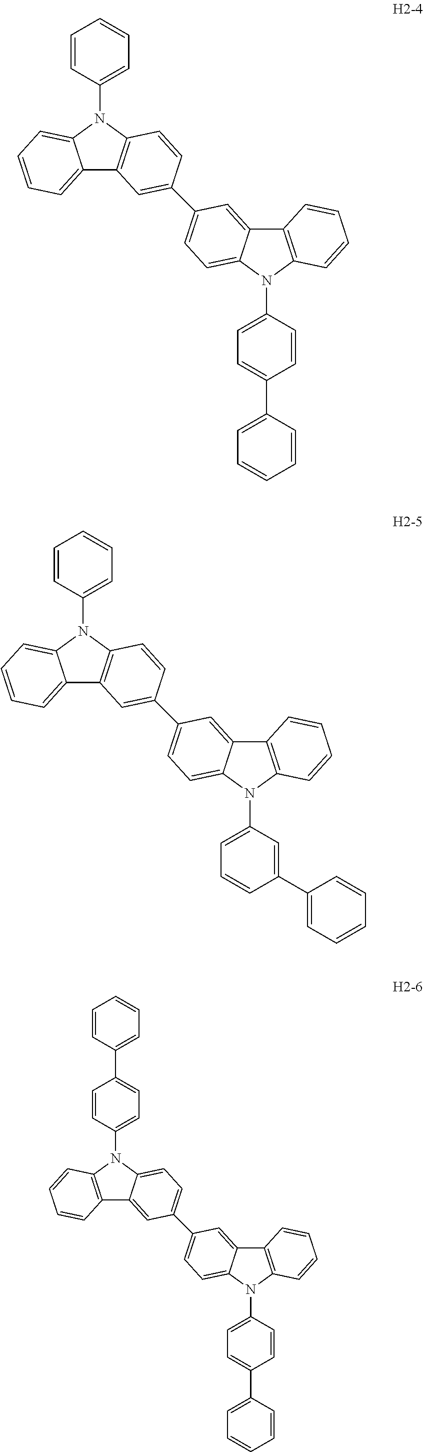 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

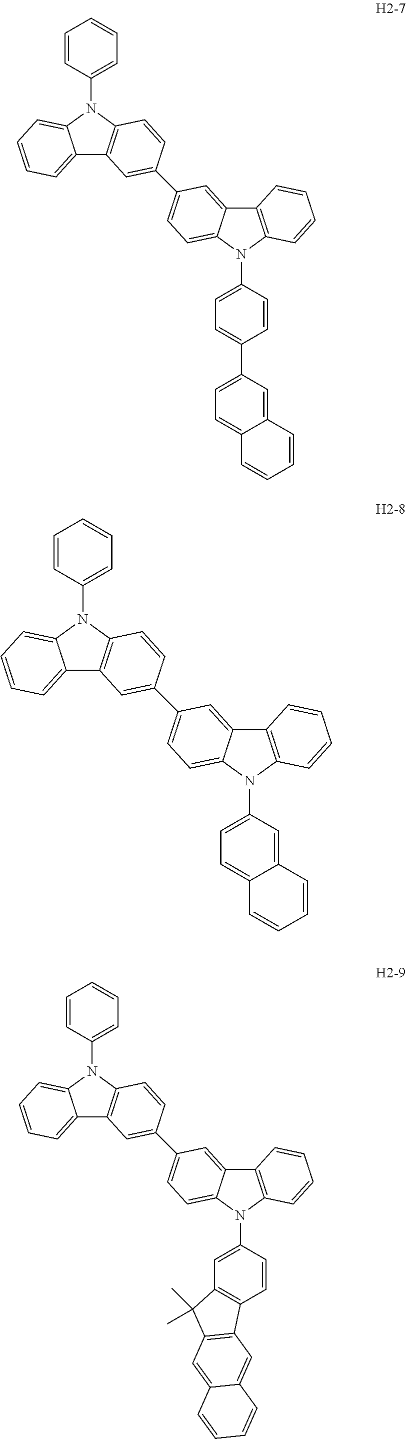 [SMILES: c1ccc(-n2c3ccccc3c3cc(-c4ccc5c(c4)c4ccccc4n5-c4ccc(-c5ccc6ccccc6c5)cc4)ccc32)cc1 ; c1ccc(-n2c3ccccc3c3cc(-c4ccc5c(c4)c4ccccc4n5-c4ccc5ccccc5c4)ccc32)cc1 ; CC1(C)c2cc(-n3c4ccccc4c4cc(-c5ccc6c(c5)c5ccccc5n6-c5ccccc5)ccc43)ccc2-c2cc3ccccc3cc21]

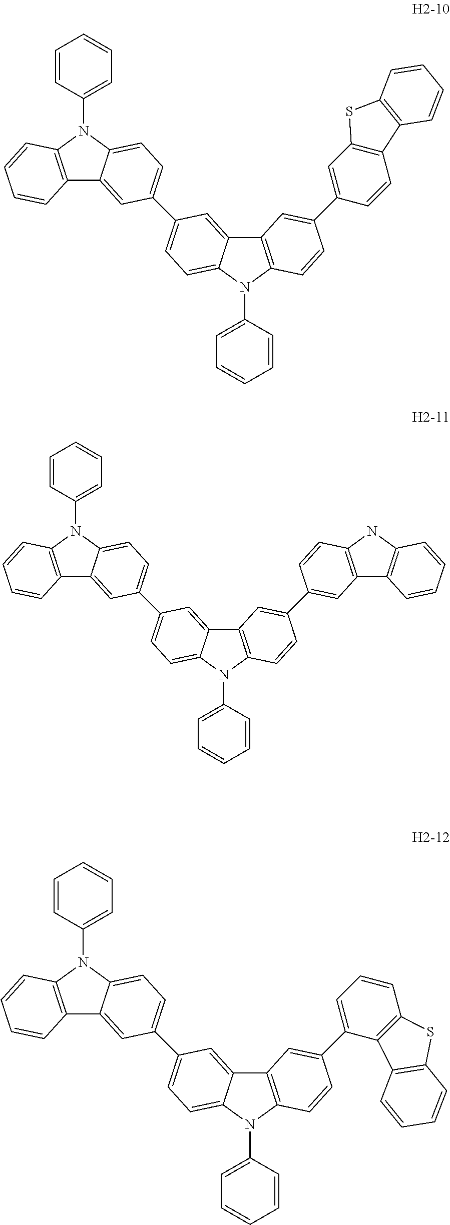 [SMILES: c1ccc(-n2c3ccccc3c3cc(-c4ccc5c(c4)c4cc(-c6ccc7c(c6)sc6ccccc67)ccc4n5-c4ccccc4)ccc32)cc1 ; c1ccc(-n2c3ccccc3c3cc(-c4ccc5c(c4)c4cc(-c6ccc7[nH]c8ccccc8c7c6)ccc4n5-c4ccccc4)ccc32)cc1 ; c1ccc(-n2c3ccccc3c3cc(-c4ccc5c(c4)c4cc(-c6cccc7sc8ccccc8c67)ccc4n5-c4ccccc4)ccc32)cc1]

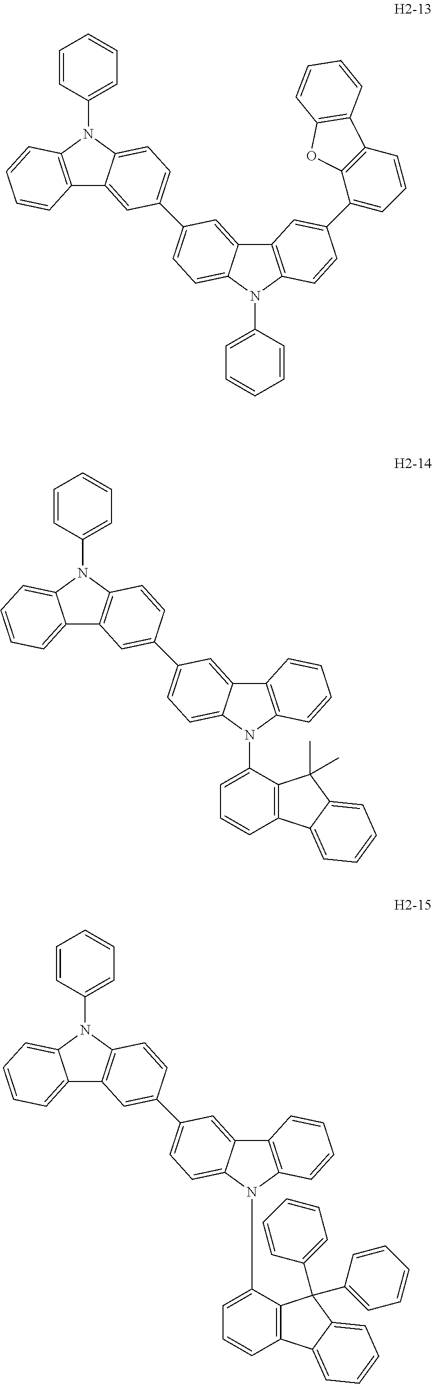 [SMILES: c1ccc(-n2c3ccccc3c3cc(-c4ccc5c(c4)c4cc(-c6cccc7c6oc6ccccc67)ccc4n5-c4ccccc4)ccc32)cc1 ; CC1(C)c2ccccc2-c2cccc(-n3c4ccccc4c4cc(-c5ccc6c(c5)c5ccccc5n6-c5ccccc5)ccc43)c21 ; c1ccc(-n2c3ccccc3c3cc(-c4ccc5c(c4)c4ccccc4n5-c4cccc5c4C(c4ccccc4)(c4ccccc4)c4ccccc4-5)ccc32)cc1]

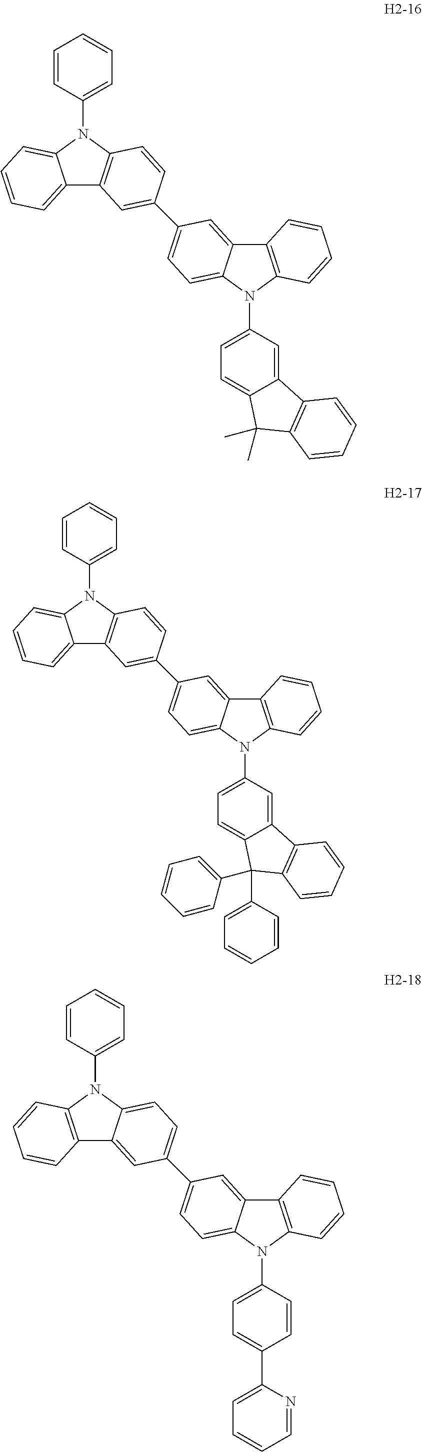 [SMILES: CC1(C)c2ccccc2-c2cc(-n3c4ccccc4c4cc(-c5ccc6c(c5)c5ccccc5n6-c5ccccc5)ccc43)ccc21 ; c1ccc(-n2c3ccccc3c3cc(-c4ccc5c(c4)c4ccccc4n5-c4ccc5c(c4)-c4ccccc4C5(c4ccccc4)c4ccccc4)ccc32)cc1 ; c1ccc(-n2c3ccccc3c3cc(-c4ccc5c(c4)c4ccccc4n5-c4ccc(-c5ccccn5)cc4)ccc32)cc1]

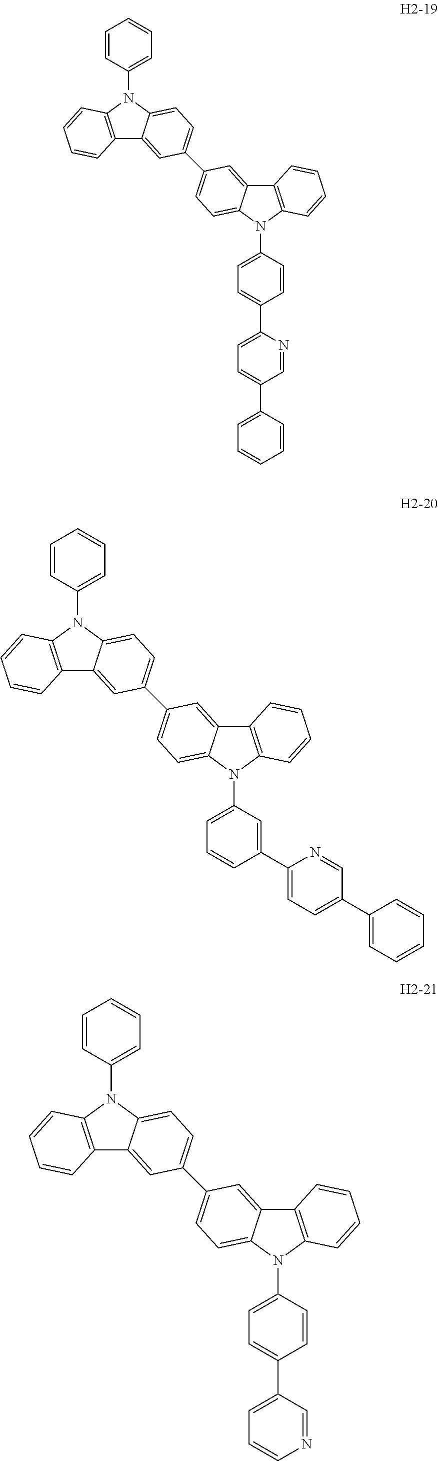 [SMILES: c1ccc(-c2ccc(-c3ccc(-n4c5ccccc5c5cc(-c6ccc7c(c6)c6ccccc6n7-c6ccccc6)ccc54)cc3)nc2)cc1 ; c1ccc(-c2ccc(-c3cccc(-n4c5ccccc5c5cc(-c6ccc7c(c6)c6ccccc6n7-c6ccccc6)ccc54)c3)nc2)cc1 ; c1ccc(-n2c3ccccc3c3cc(-c4ccc5c(c4)c4ccccc4n5-c4ccc(-c5cccnc5)cc4)ccc32)cc1]

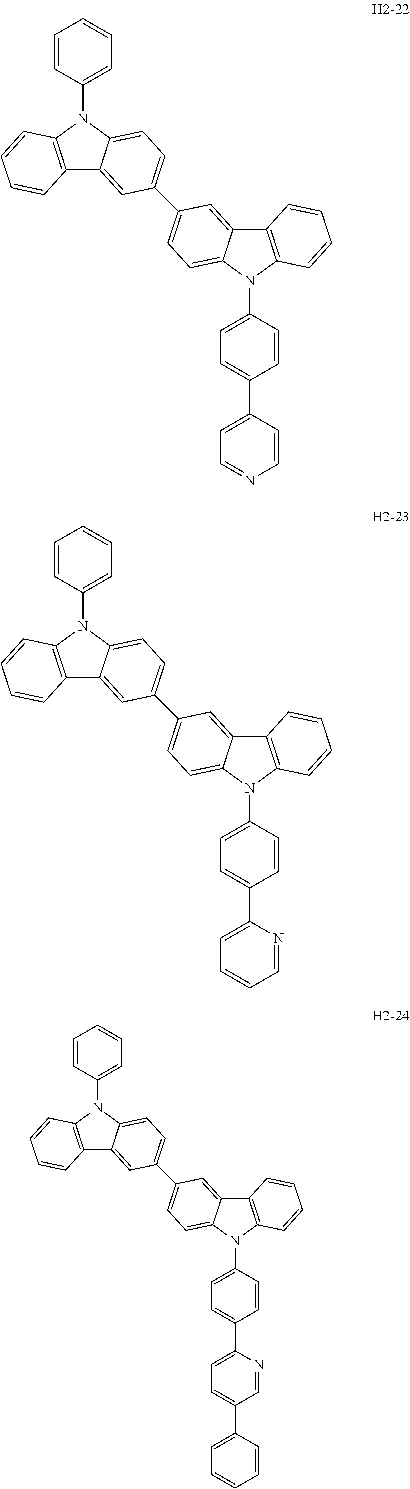 [SMILES: c1ccc(-n2c3ccccc3c3cc(-c4ccc5c(c4)c4ccccc4n5-c4ccc(-c5ccncc5)cc4)ccc32)cc1 ; c1ccc(-n2c3ccccc3c3cc(-c4ccc5c(c4)c4ccccc4n5-c4ccc(-c5ccccn5)cc4)ccc32)cc1 ; c1ccc(-c2ccc(-c3ccc(-n4c5ccccc5c5cc(-c6ccc7c(c6)c6ccccc6n7-c6ccccc6)ccc54)cc3)nc2)cc1]

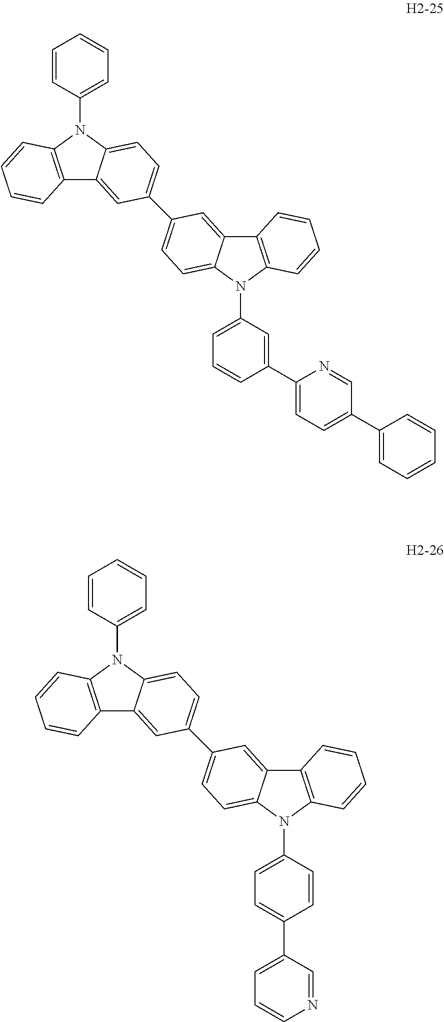 [SMILES: c1ccc(-c2ccc(-c3cccc(-n4c5ccccc5c5cc(-c6ccc7c(c6)c6ccccc6n7-c6ccccc6)ccc54)c3)nc2)cc1 ; c1ccc(-n2c3ccccc3c3cc(-c4ccc5c(c4)c4ccccc4n5-c4ccc(-c5cccnc5)cc4)ccc32)cc1]

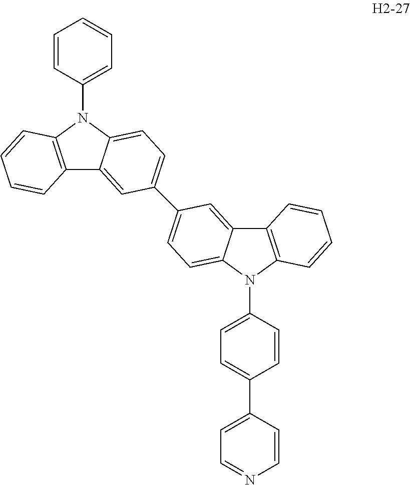 [SMILES: c1ccc(-n2c3ccccc3c3cc(-c4ccc5c(c4)c4ccccc4n5-c4ccc(-c5ccncc5)cc4)ccc32)cc1]

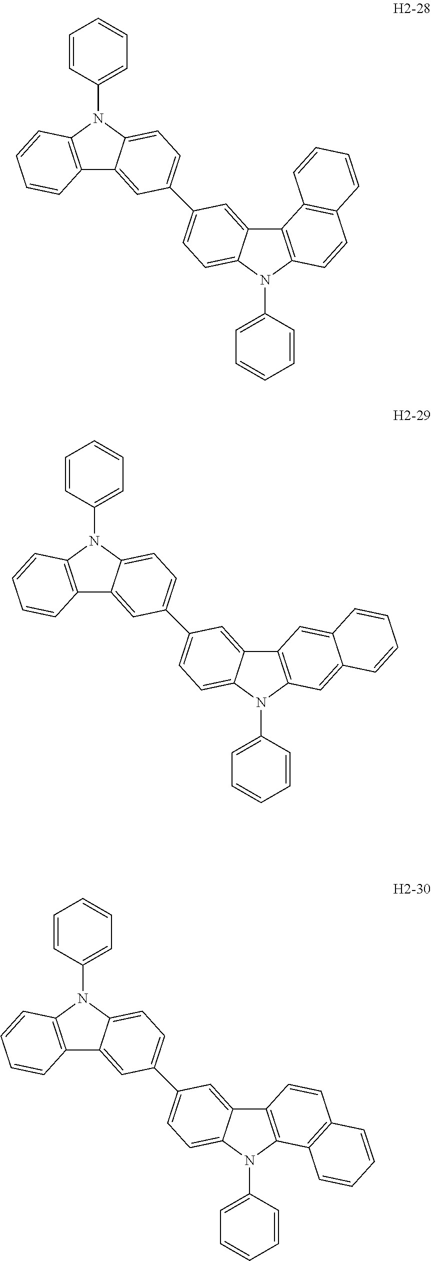 [SMILES: c1ccc(-n2c3ccccc3c3cc(-c4ccc5c(c4)c4c6ccccc6ccc4n5-c4ccccc4)ccc32)cc1 ; c1ccc(-n2c3ccccc3c3cc(-c4ccc5c(c4)c4cc6ccccc6cc4n5-c4ccccc4)ccc32)cc1 ; c1ccc(-n2c3ccccc3c3cc(-c4ccc5c(c4)c4ccc6ccccc6c4n5-c4ccccc4)ccc32)cc1]

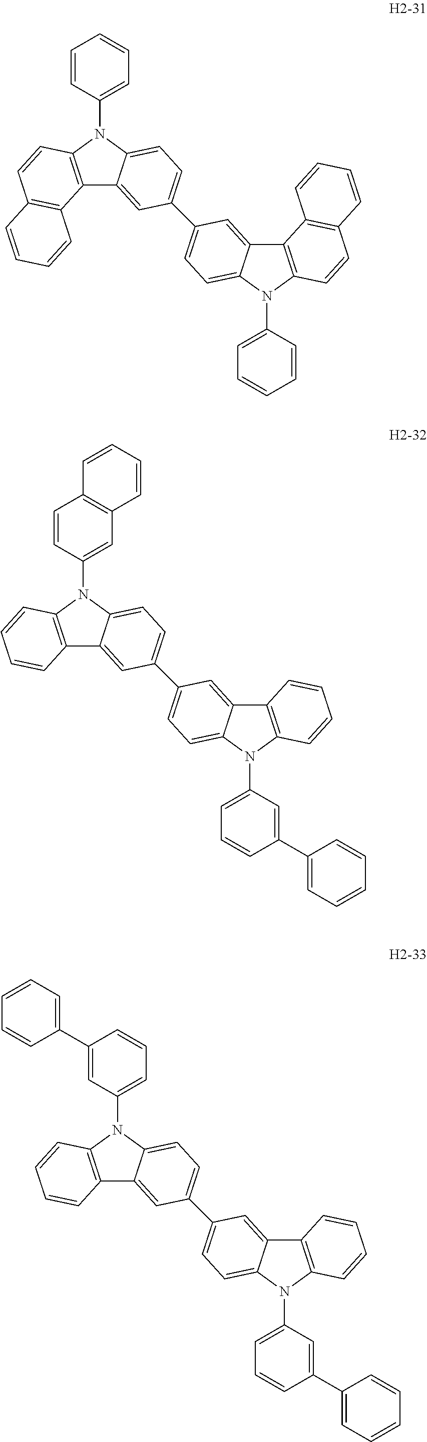 [SMILES: c1ccc(-n2c3ccc(-c4ccc5c(c4)c4c6ccccc6ccc4n5-c4ccccc4)cc3c3c4ccccc4ccc32)cc1 ; c1ccc(-c2cccc(-n3c4ccccc4c4cc(-c5ccc6c(c5)c5ccccc5n6-c5ccc6ccccc6c5)ccc43)c2)cc1 ; c1ccc(-c2cccc(-n3c4ccccc4c4cc(-c5ccc6c(c5)c5ccccc5n6-c5cccc(-c6ccccc6)c5)ccc43)c2)cc1]

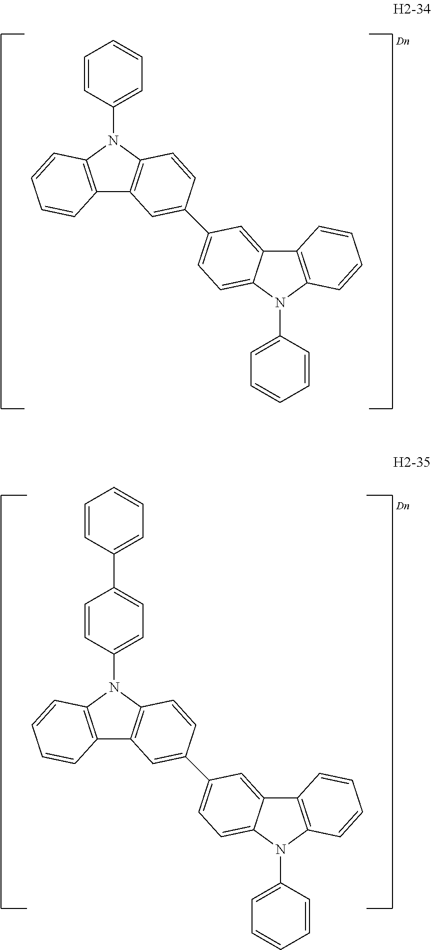

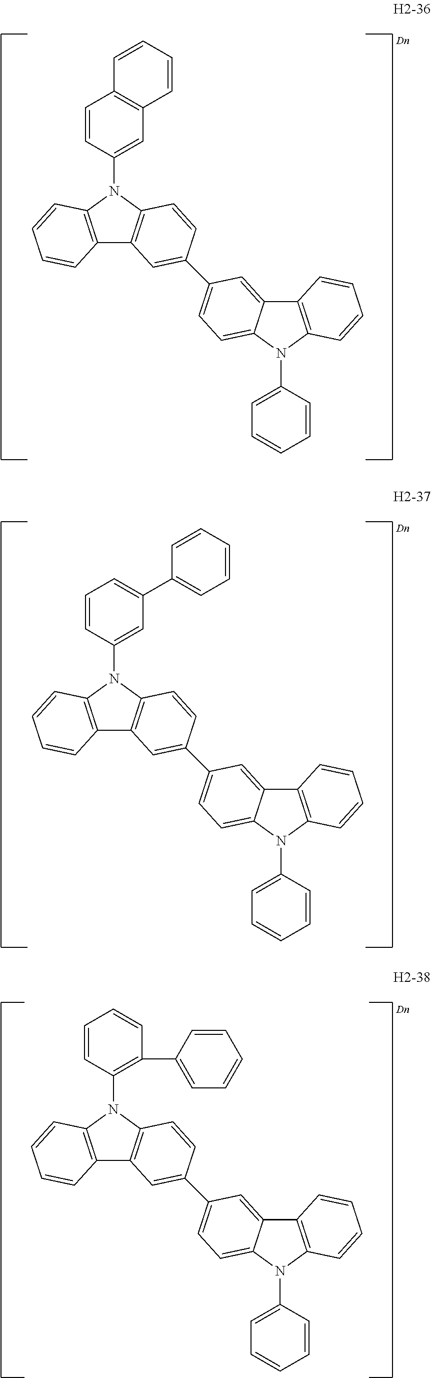

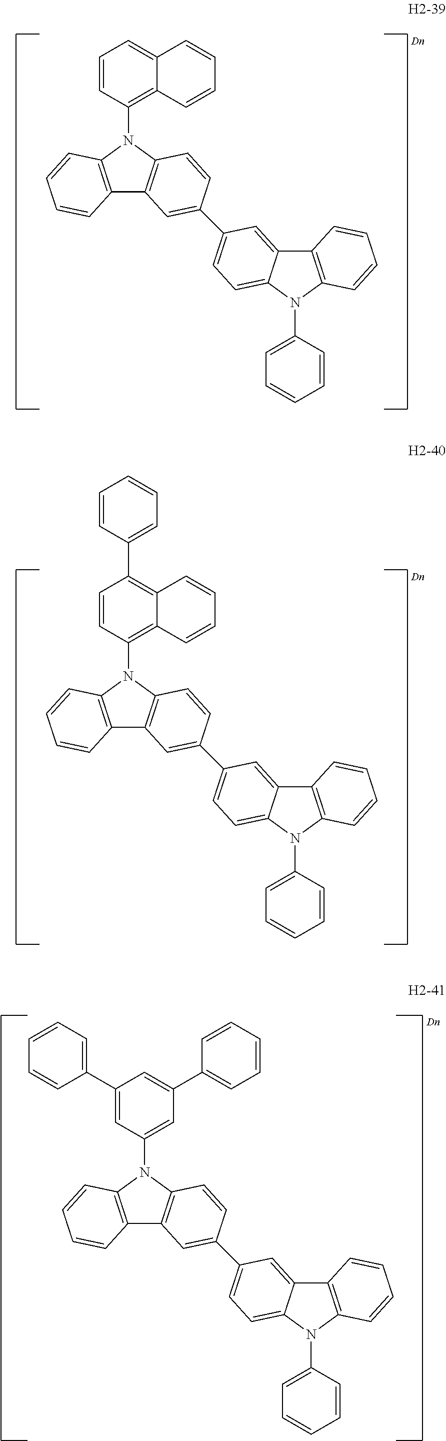

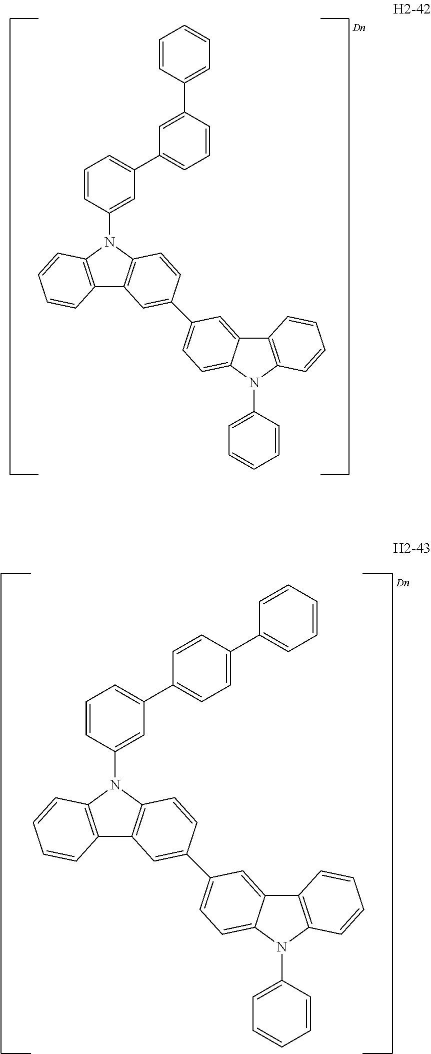

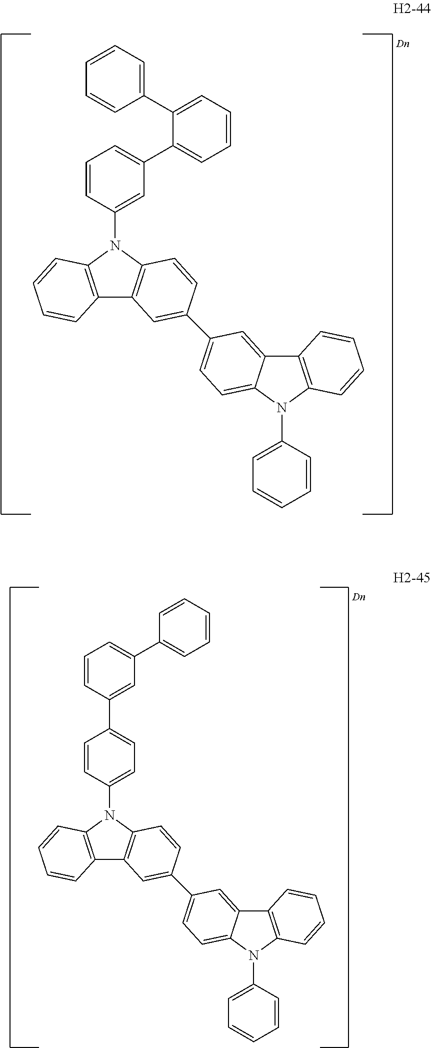

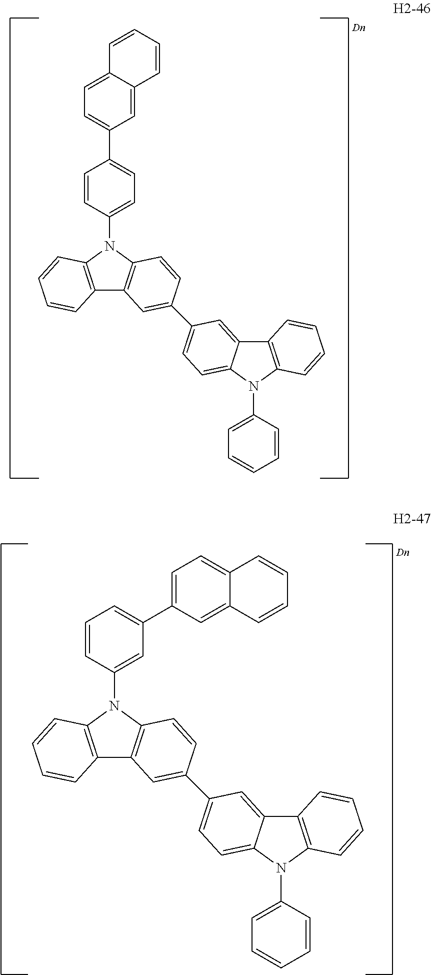

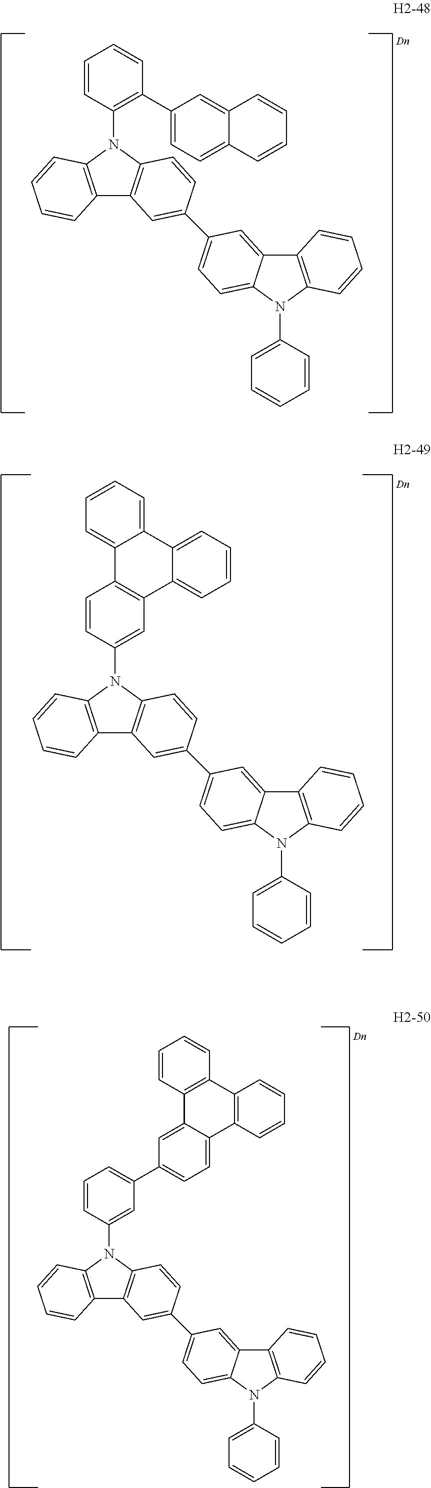

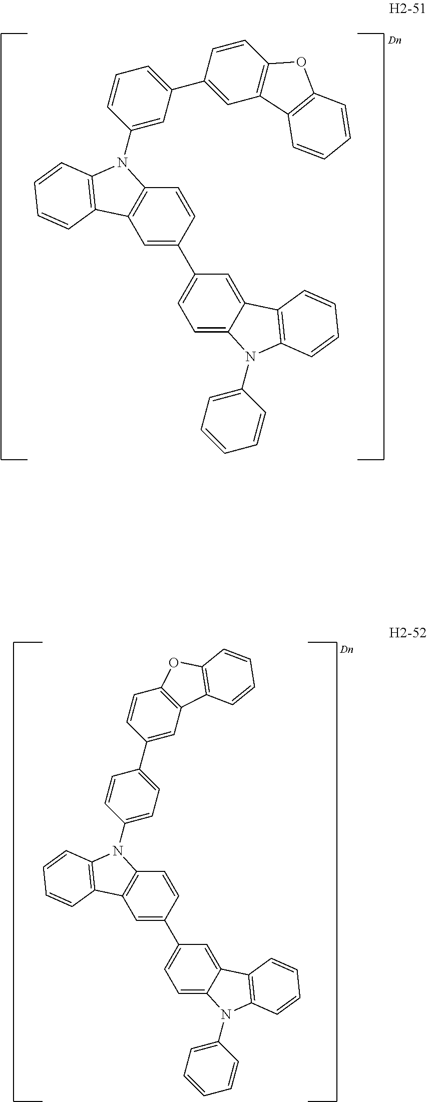

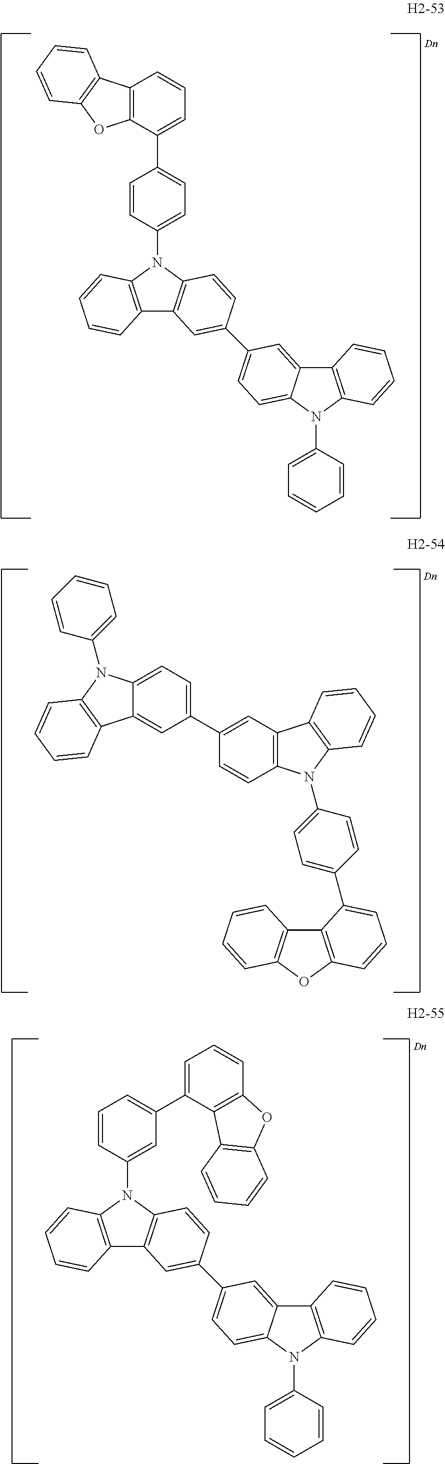

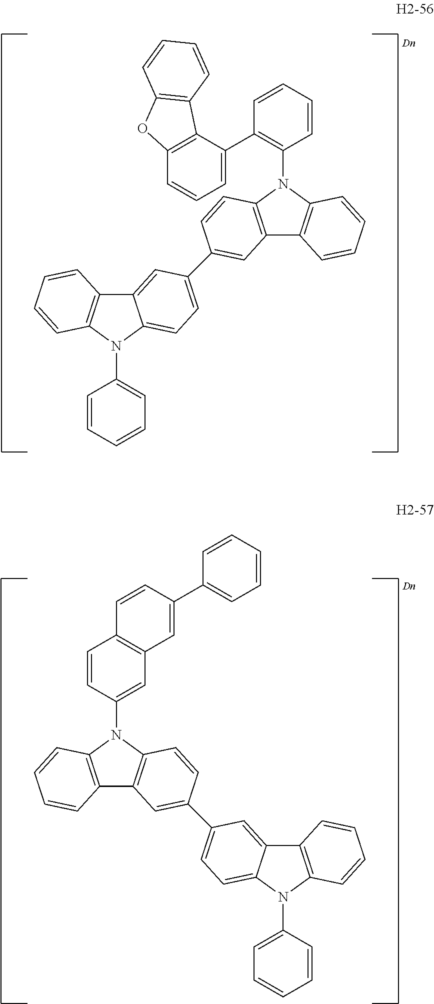

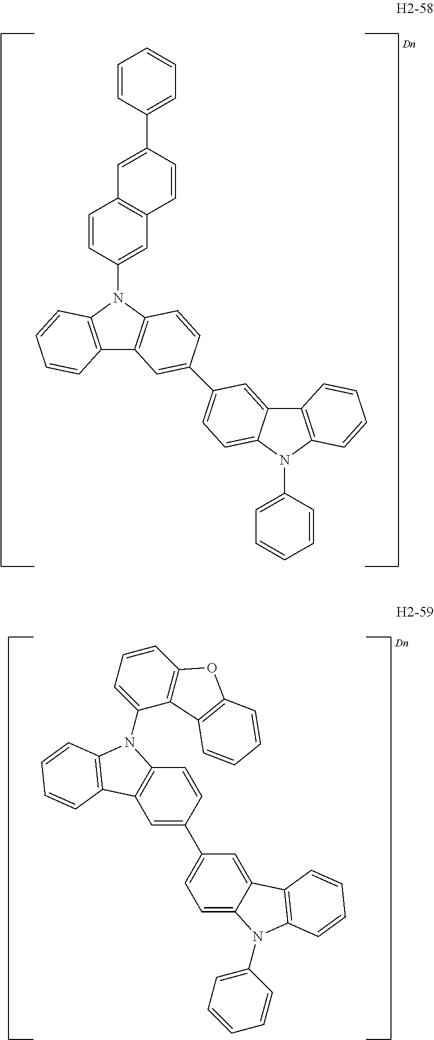

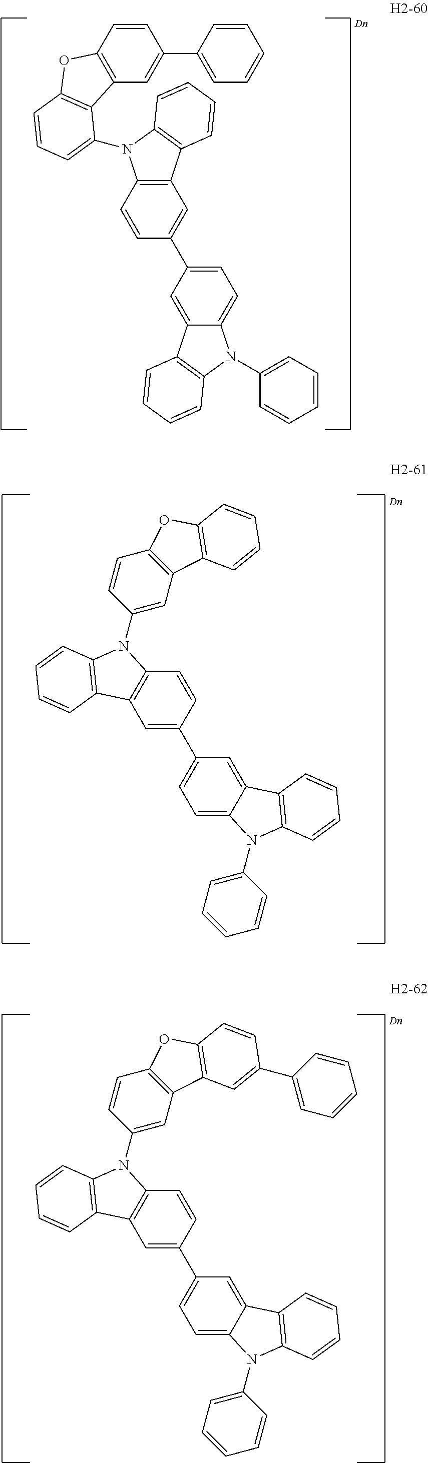

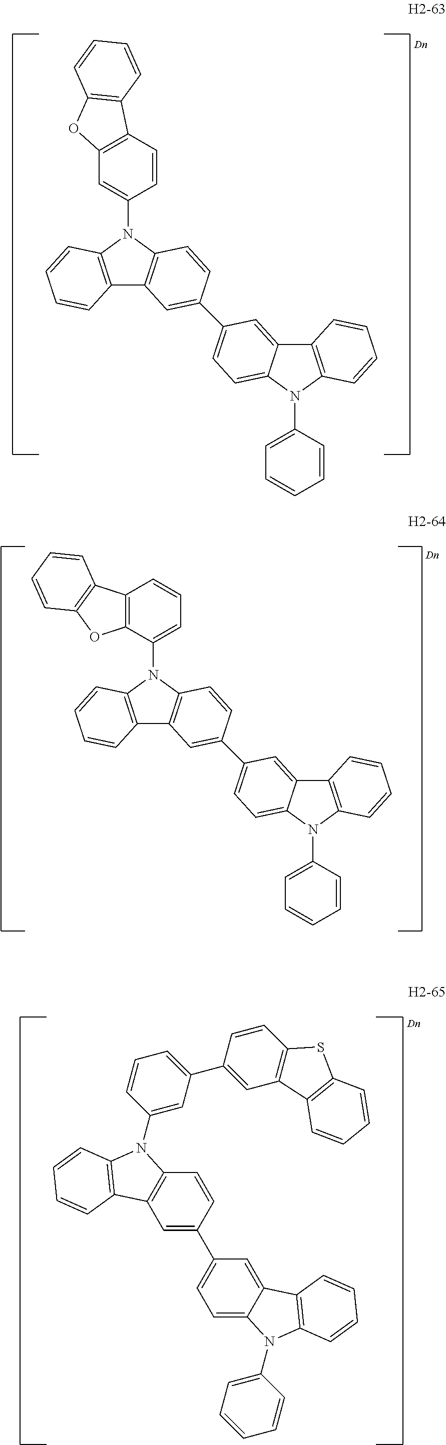

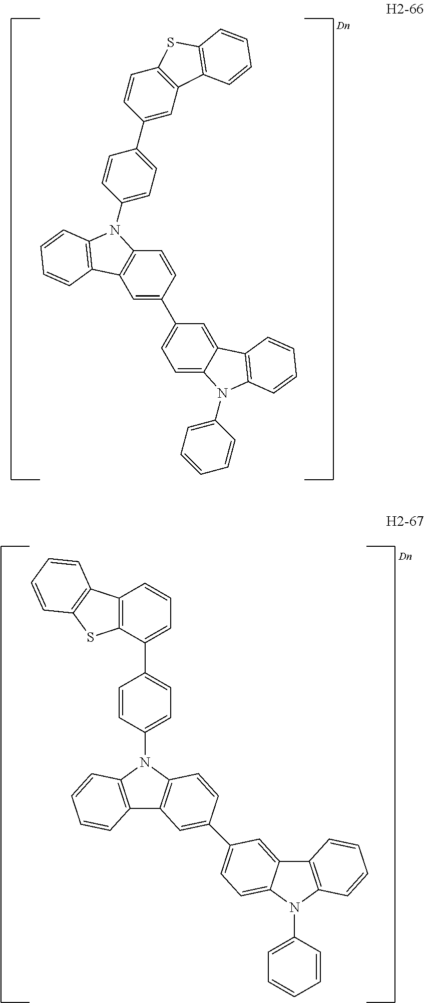

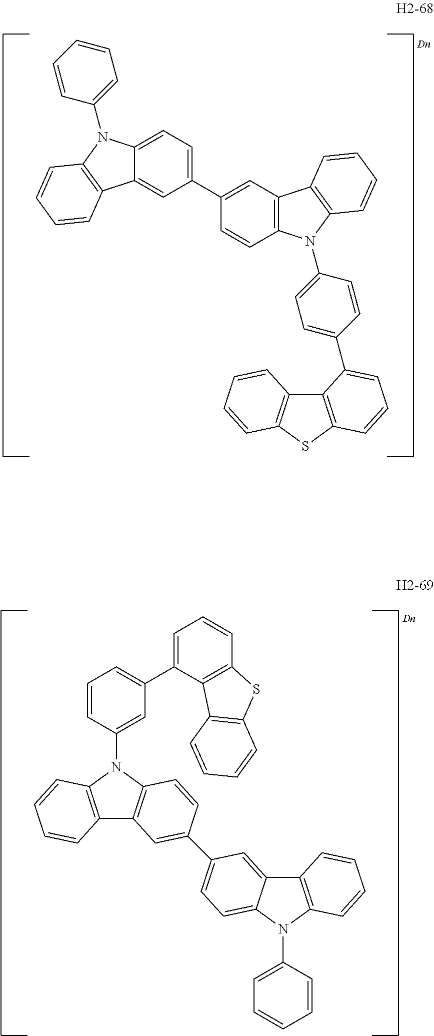

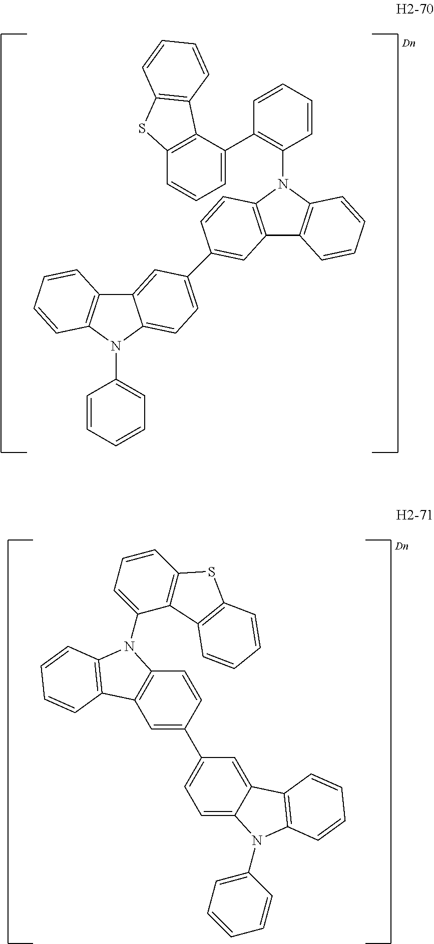

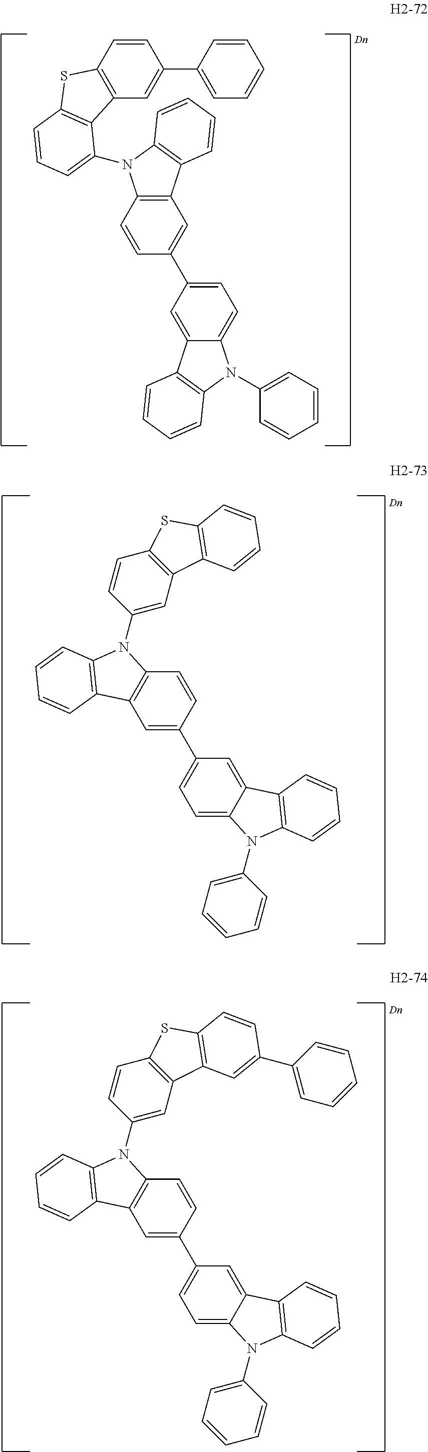

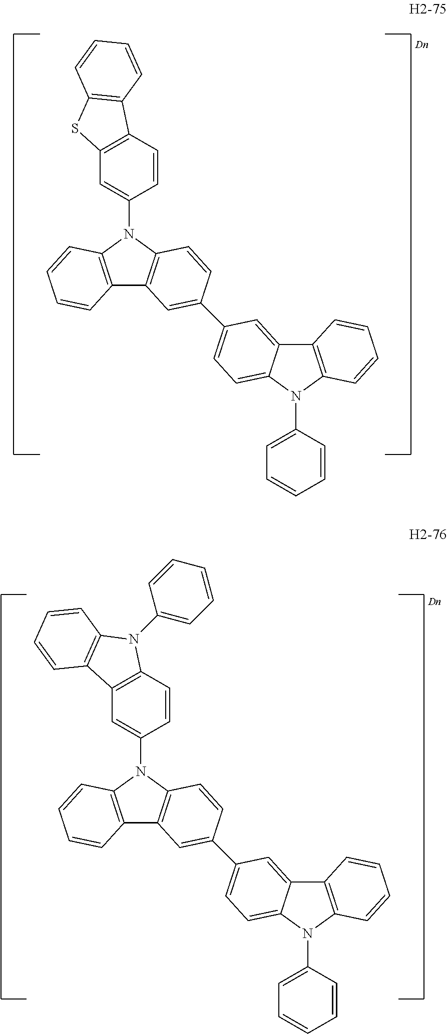

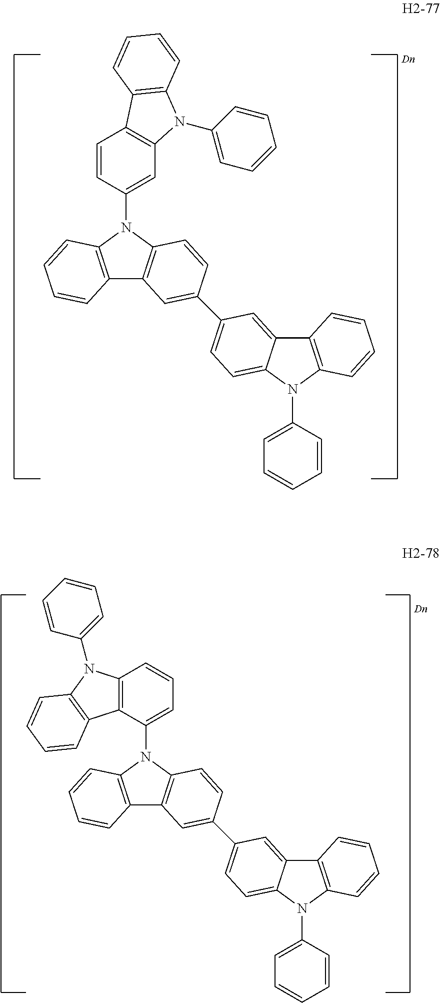

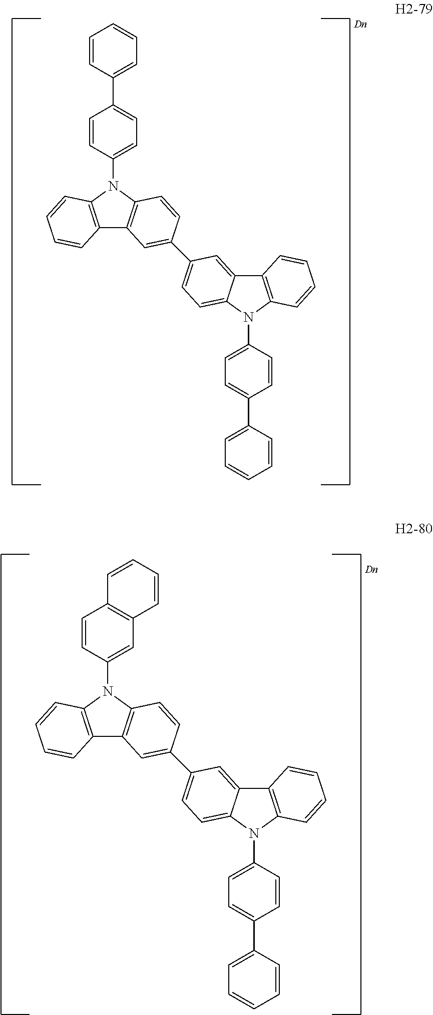

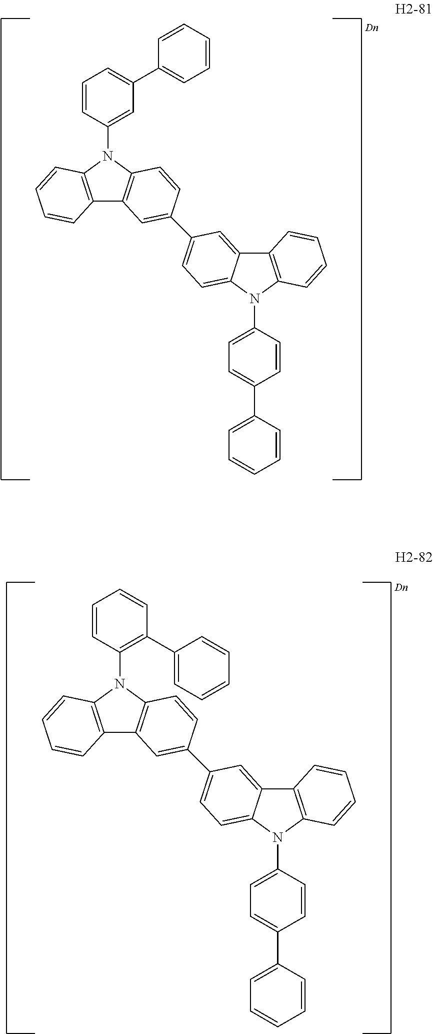

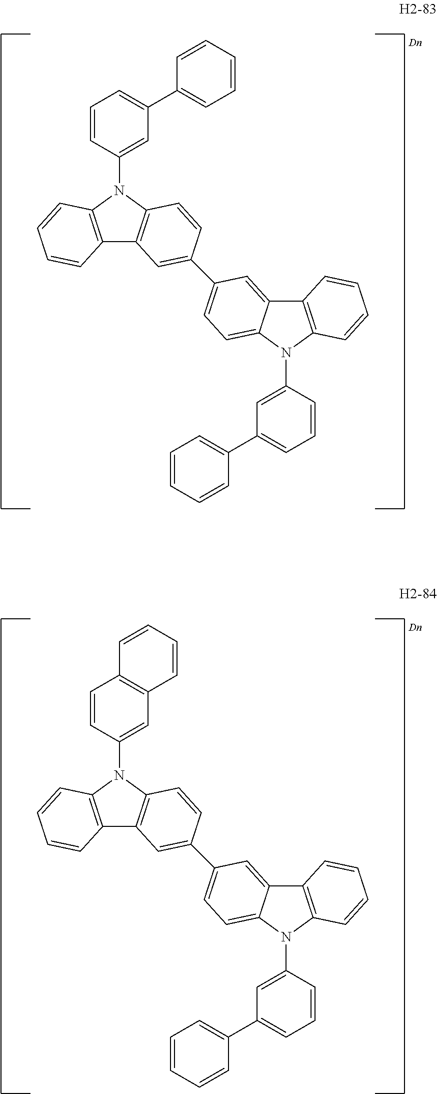

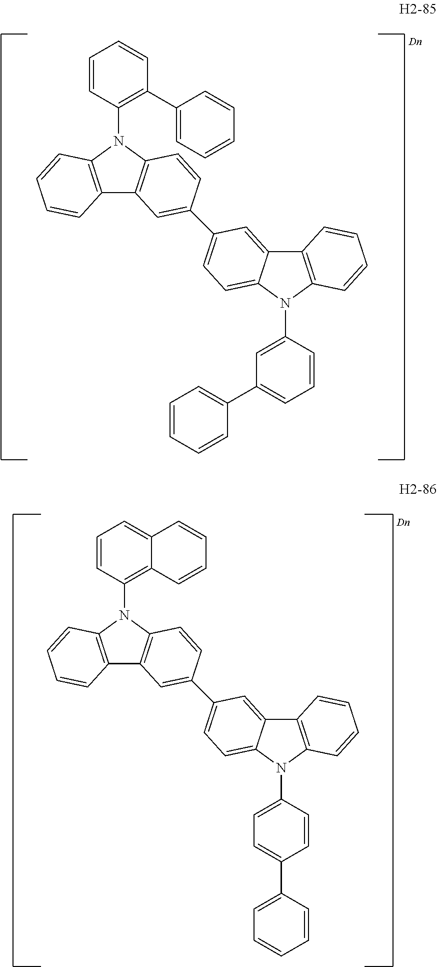

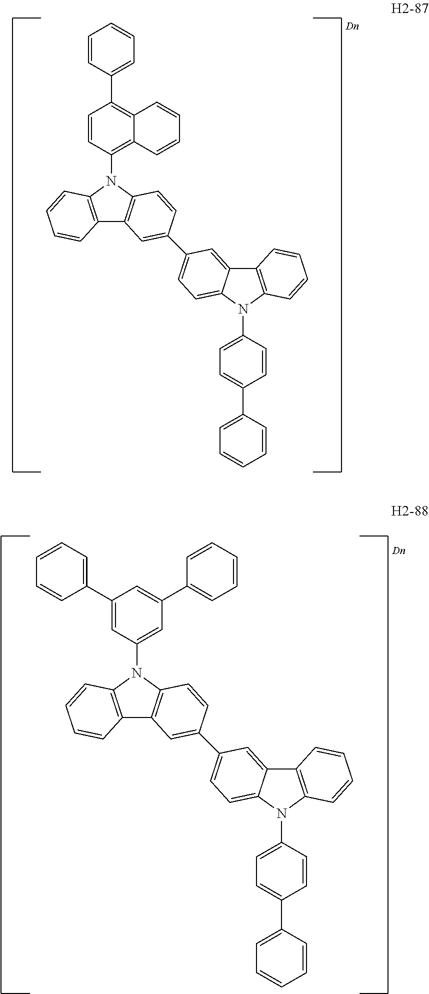

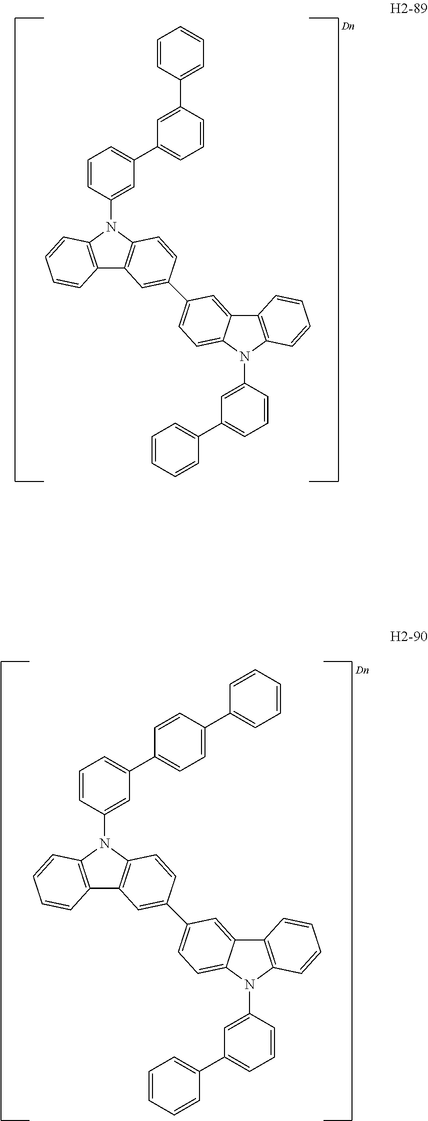

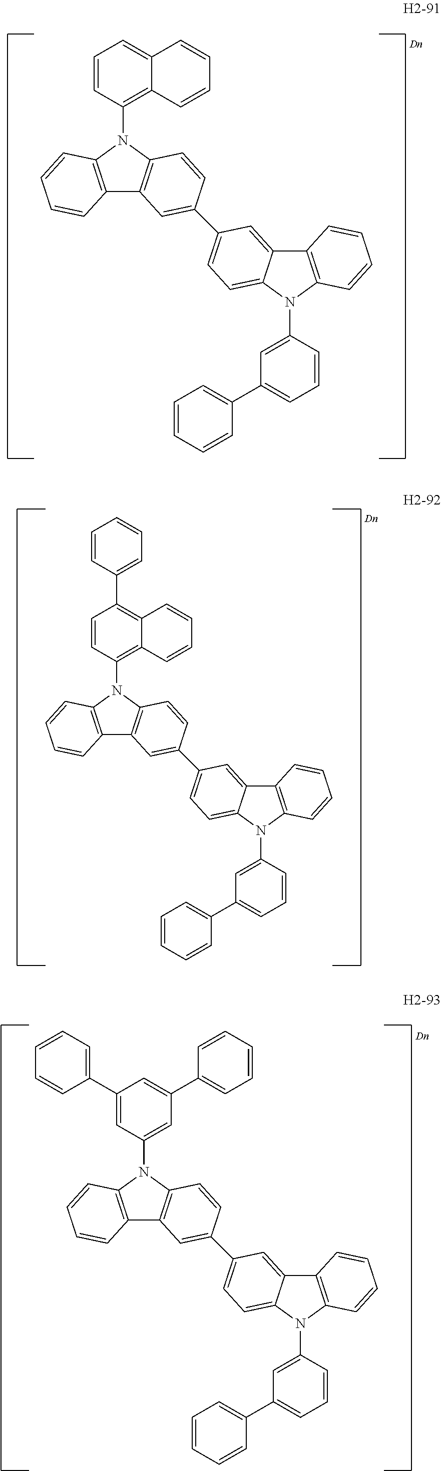

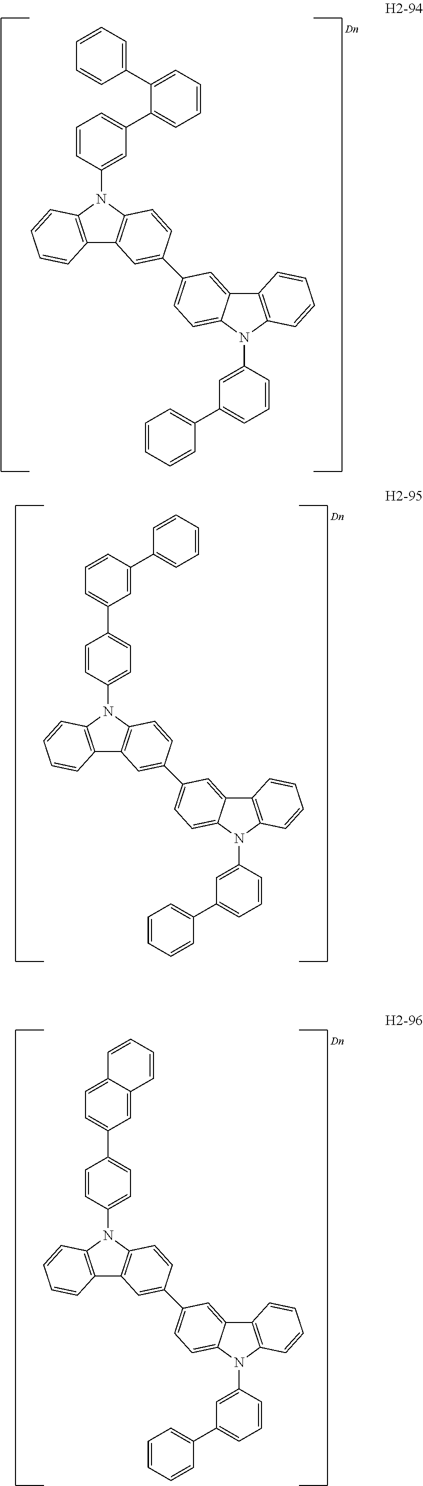

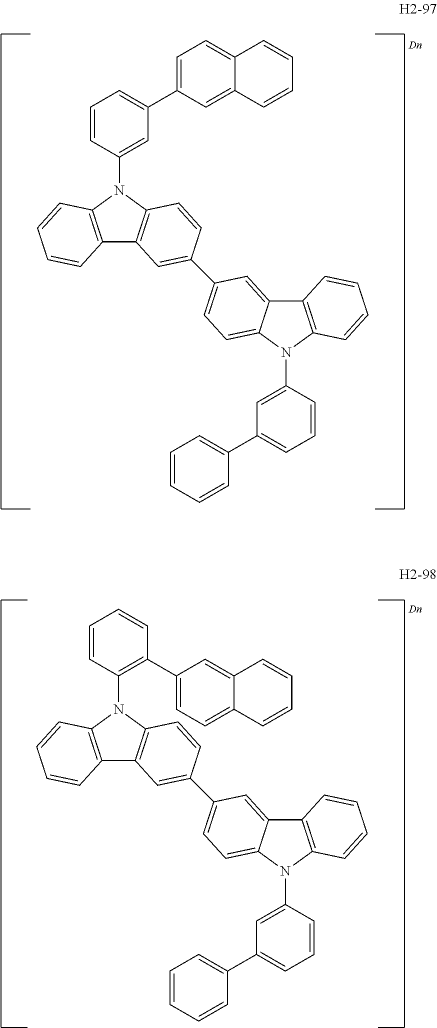

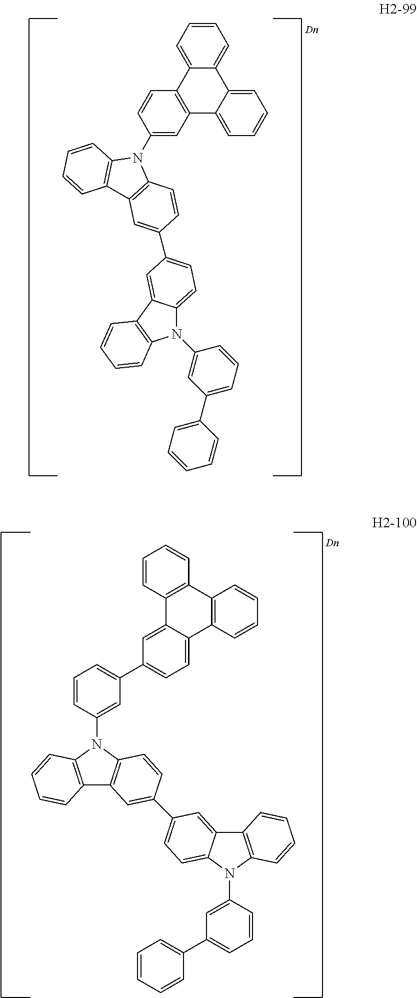

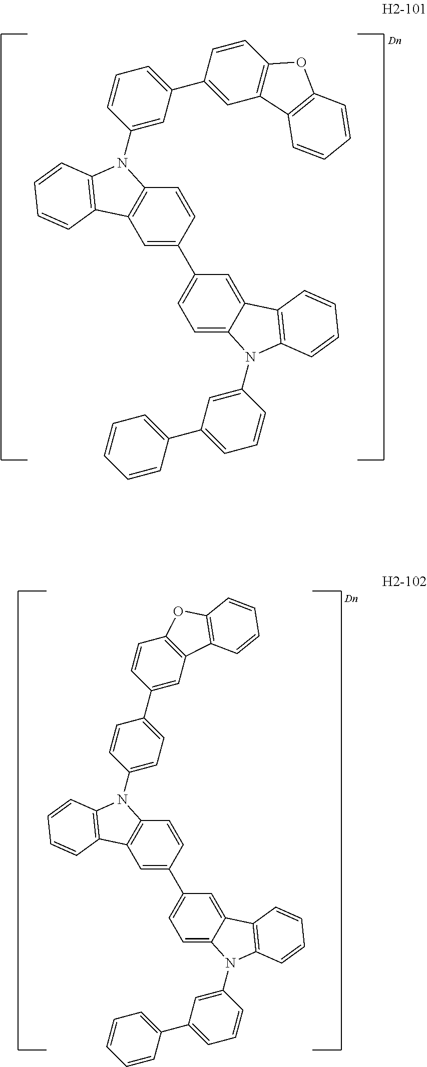

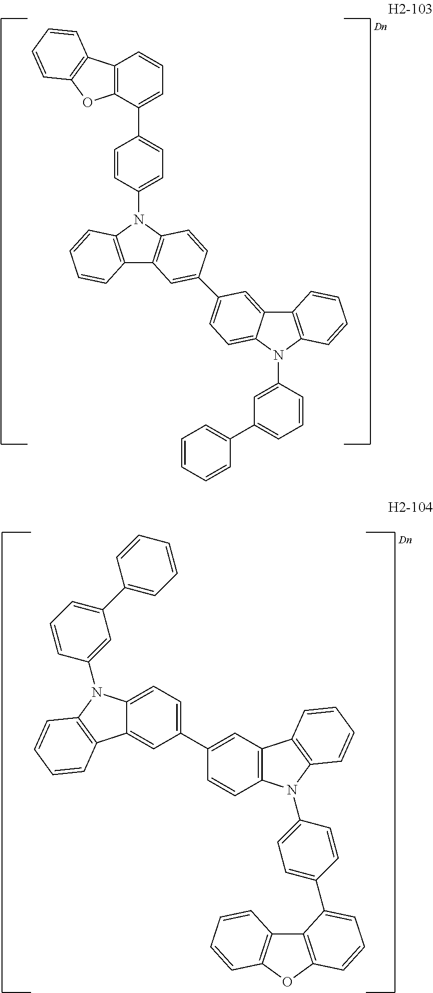

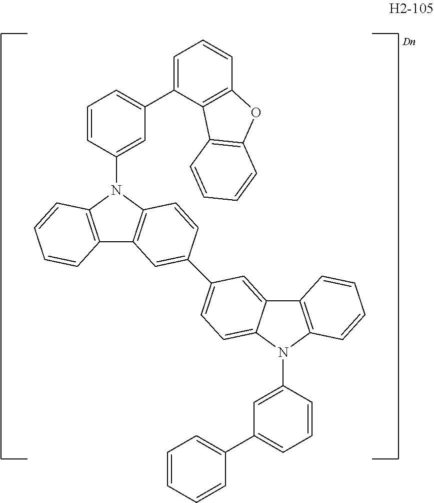

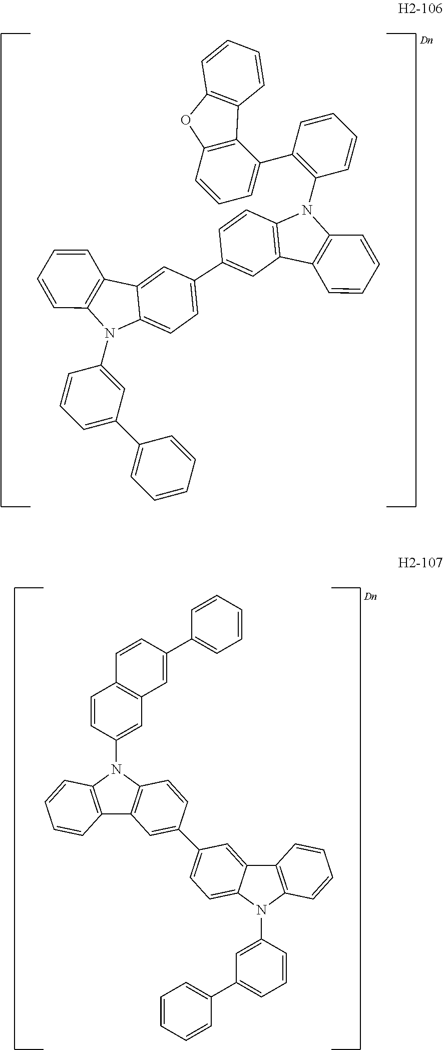

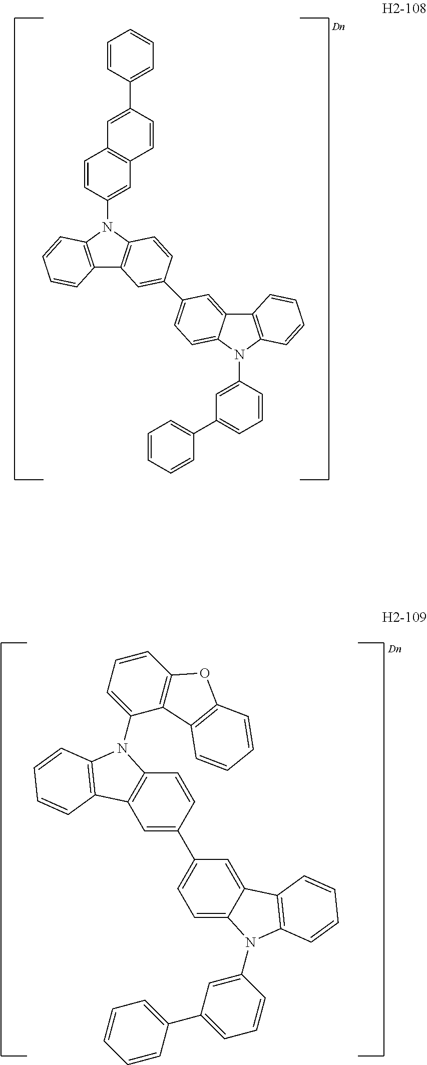

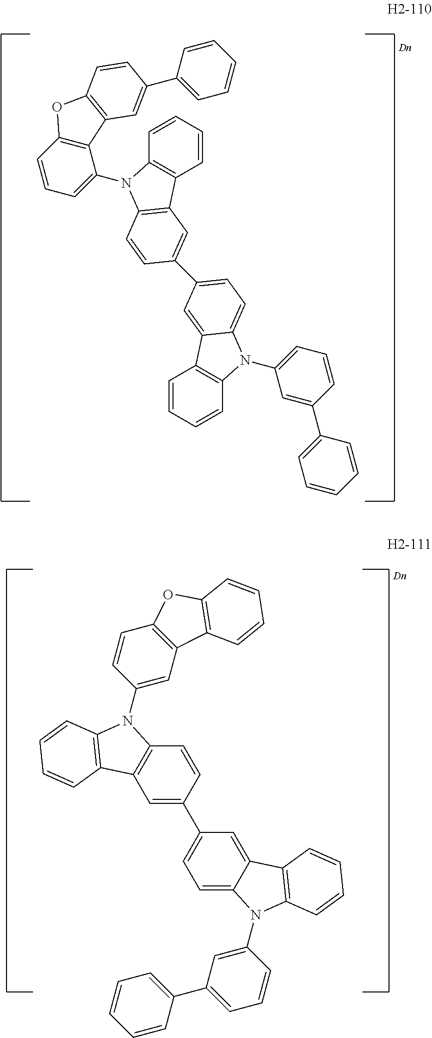

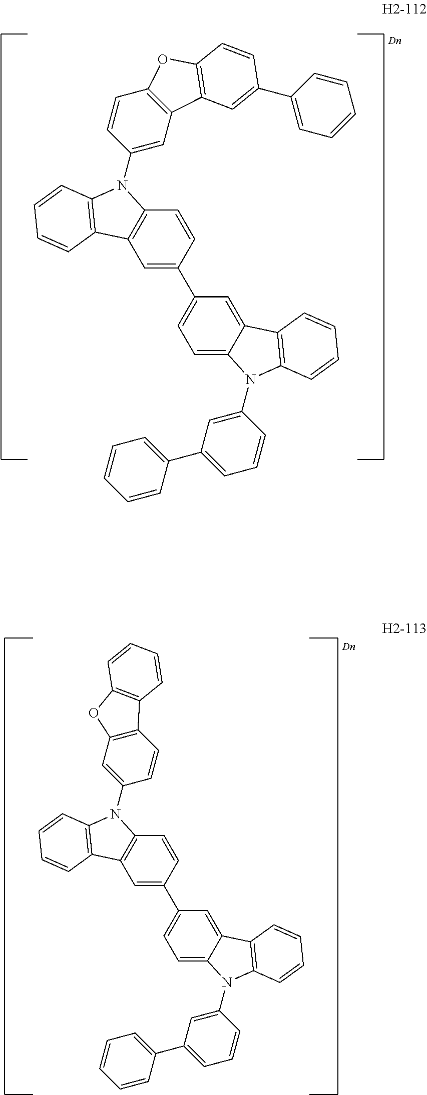

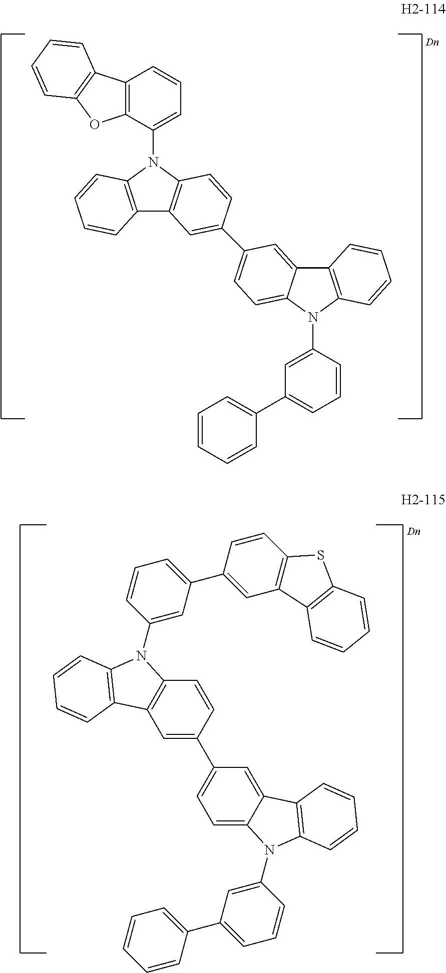

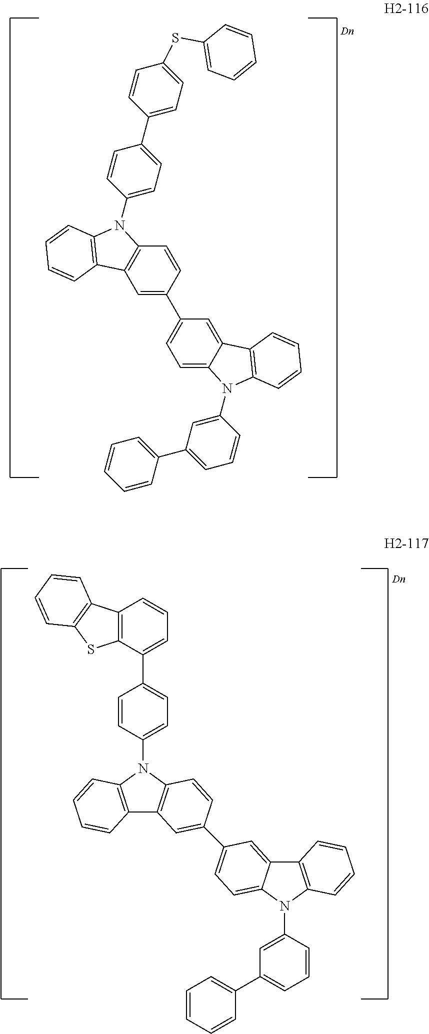

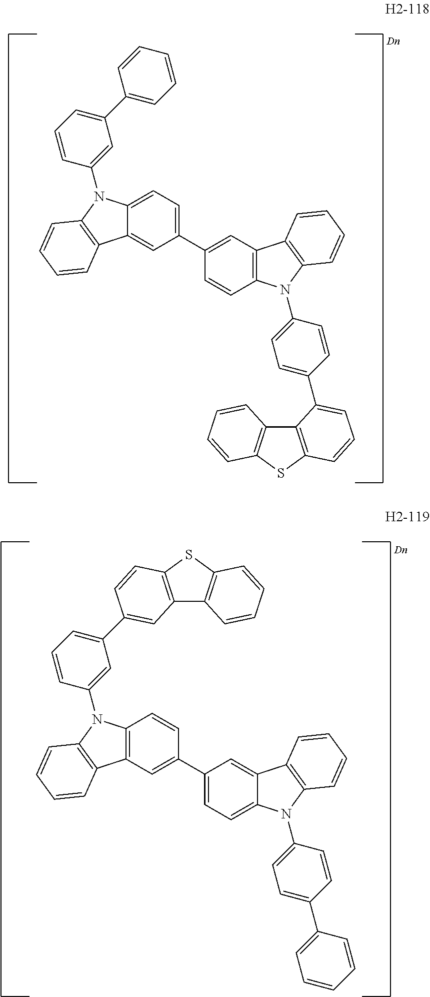

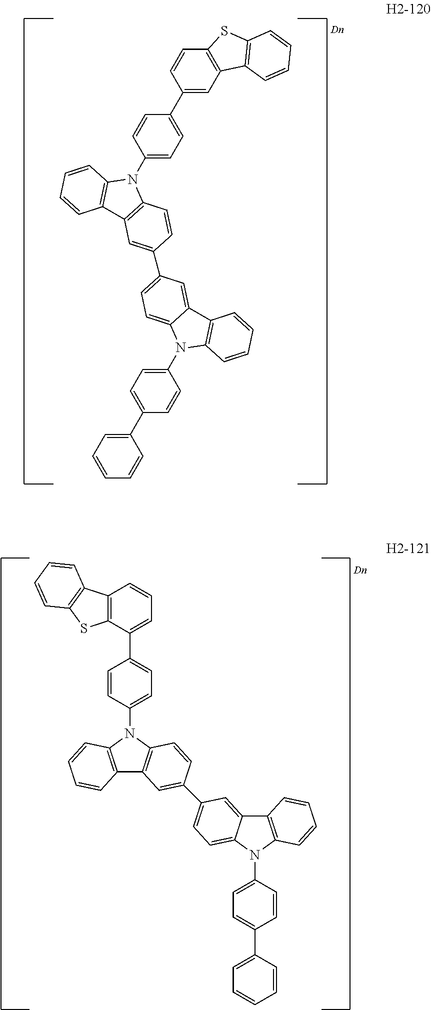

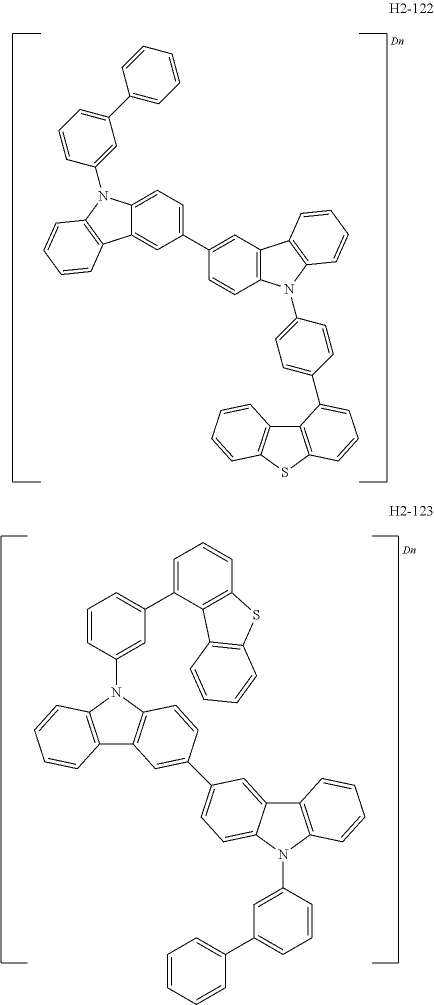

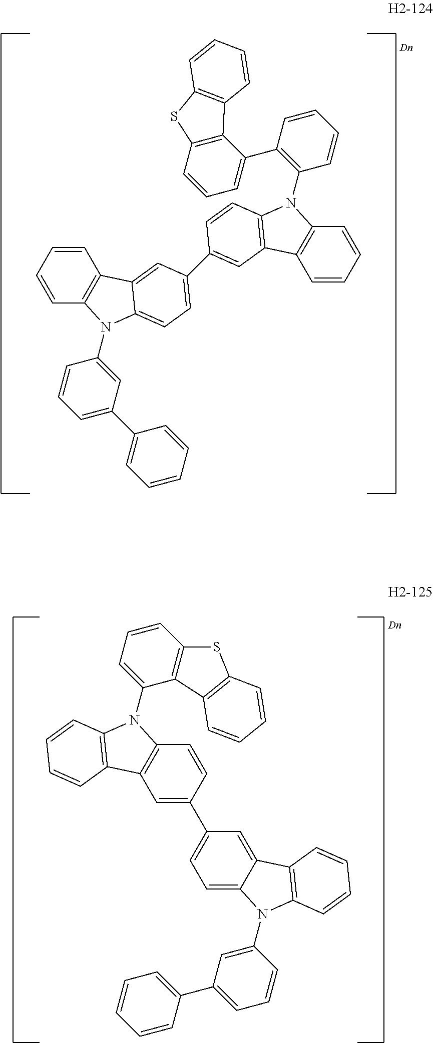

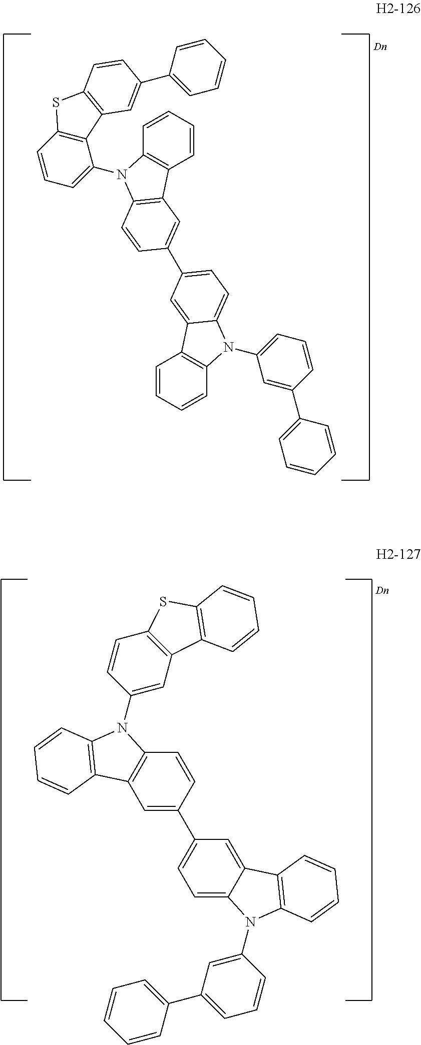

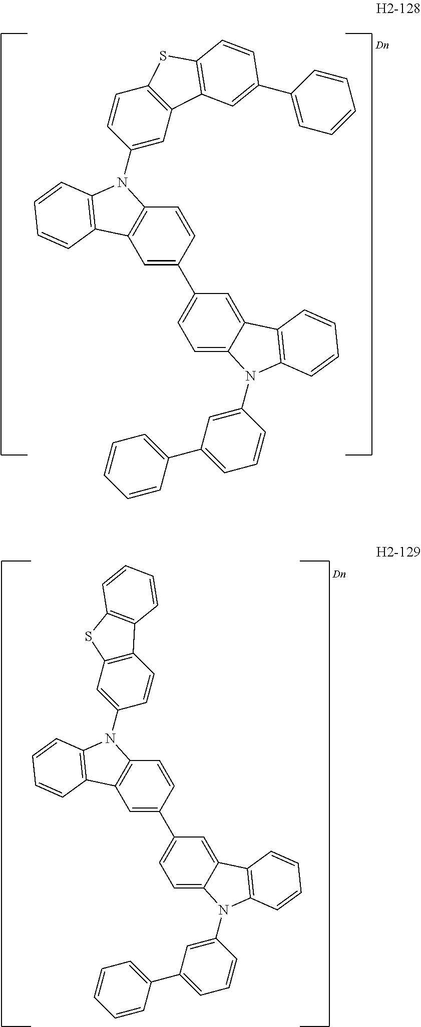

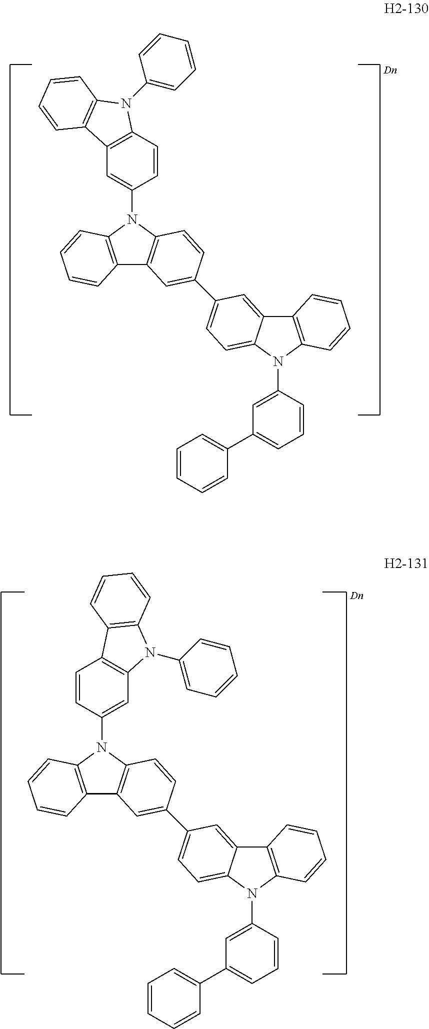

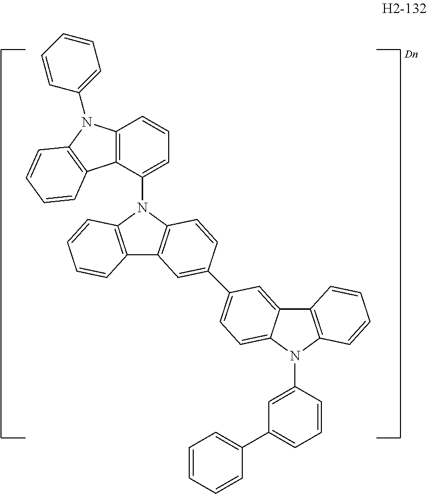

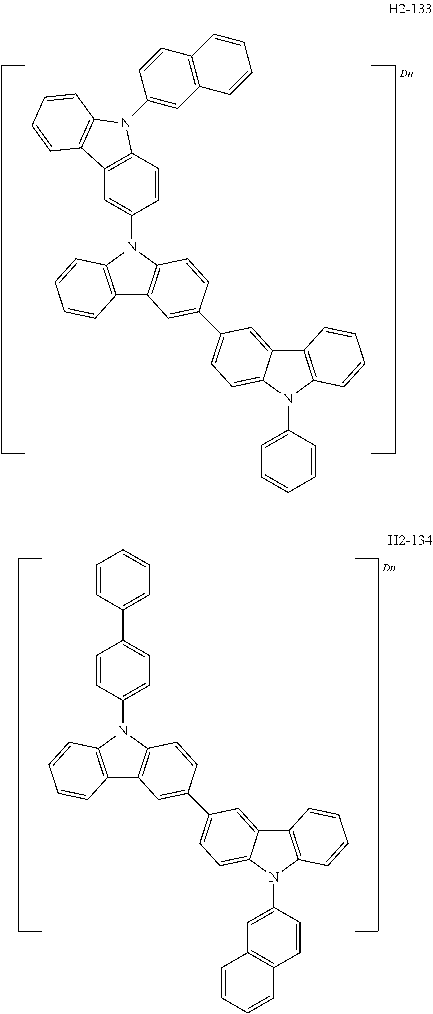

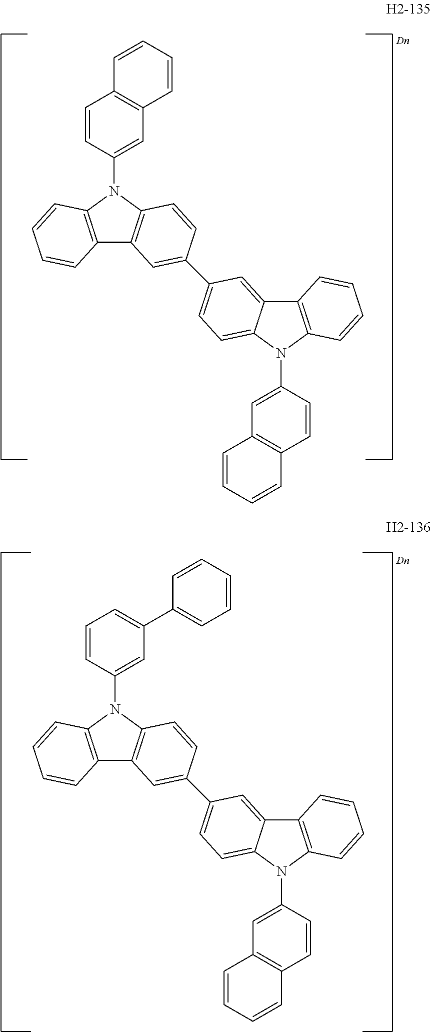

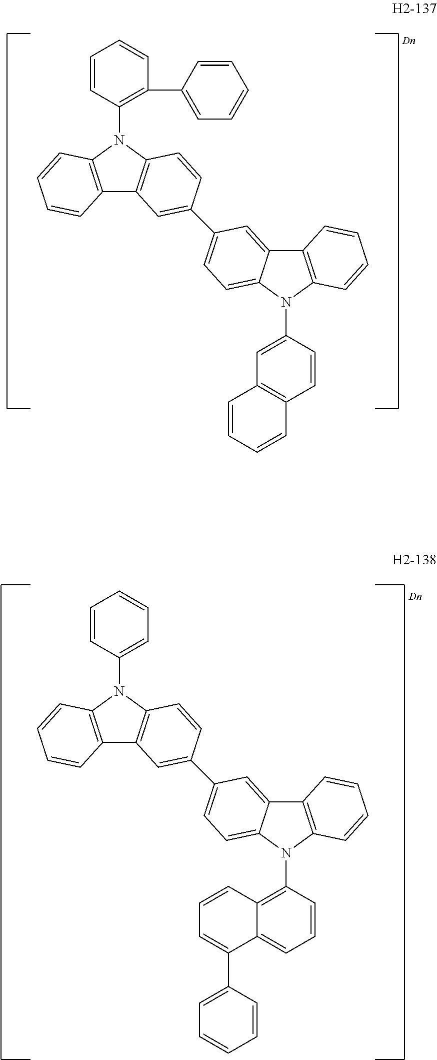

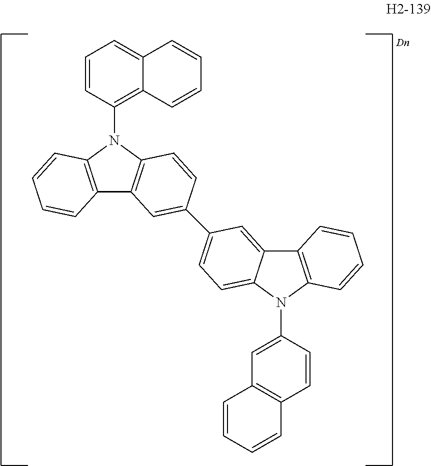

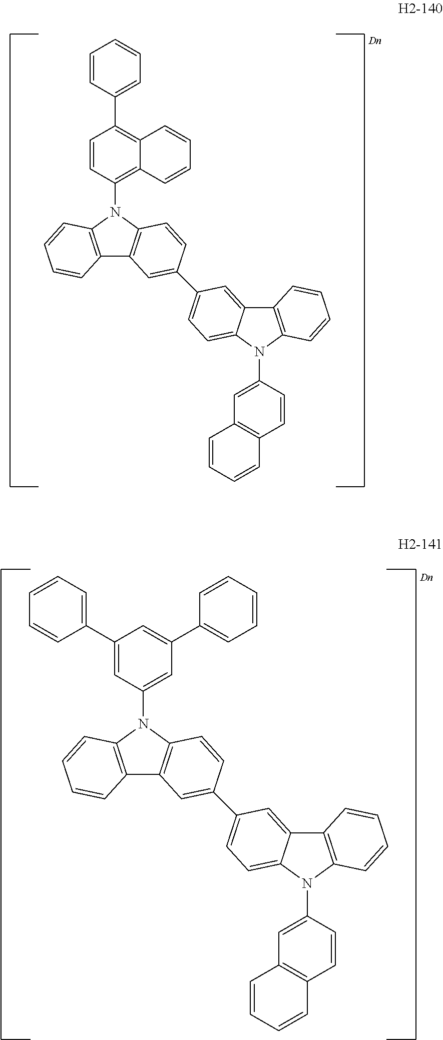

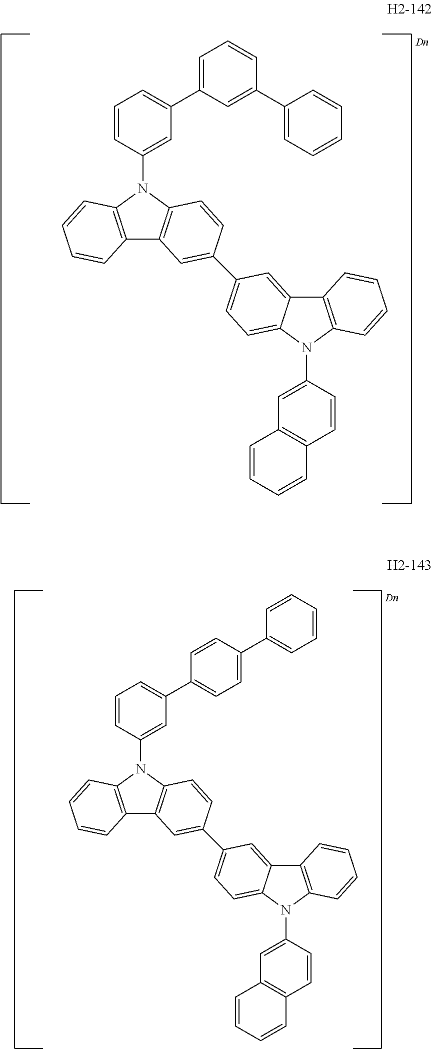

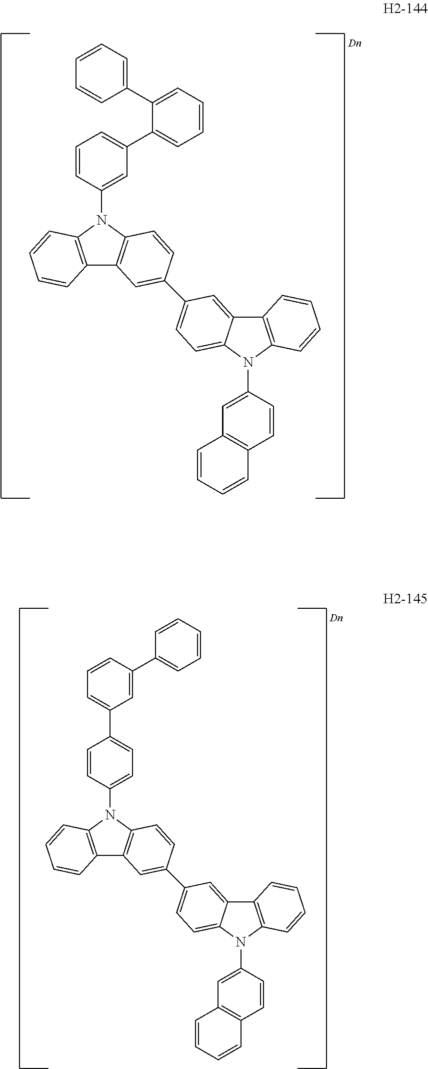

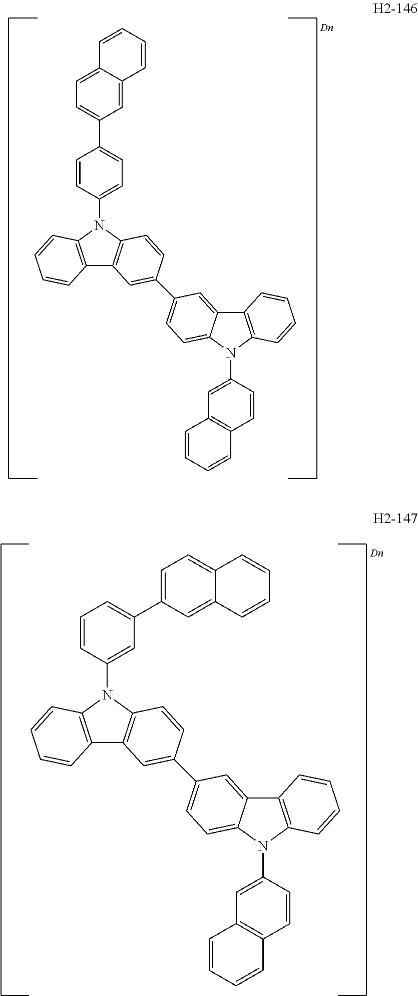

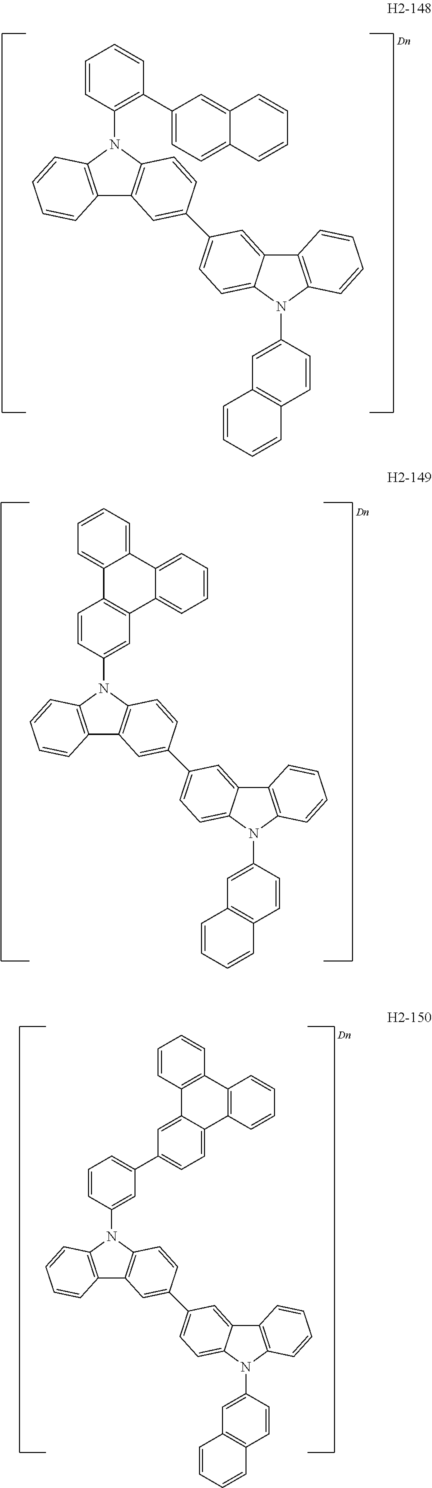

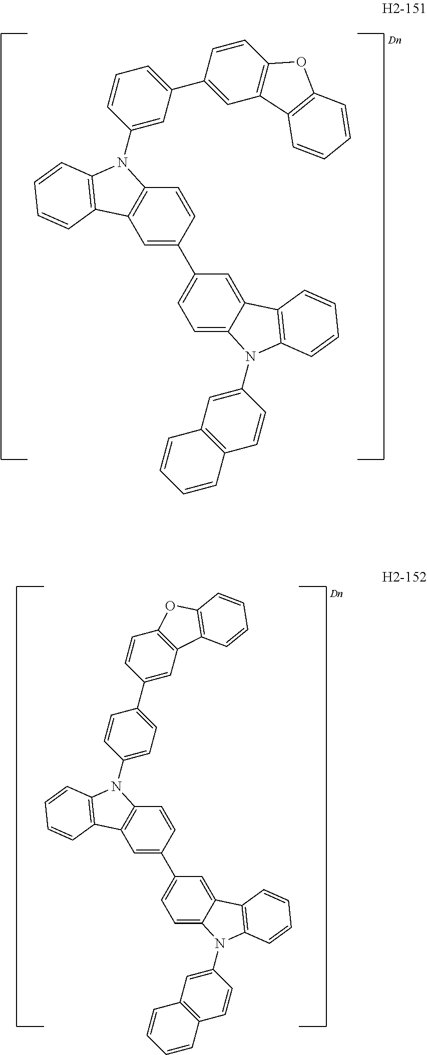

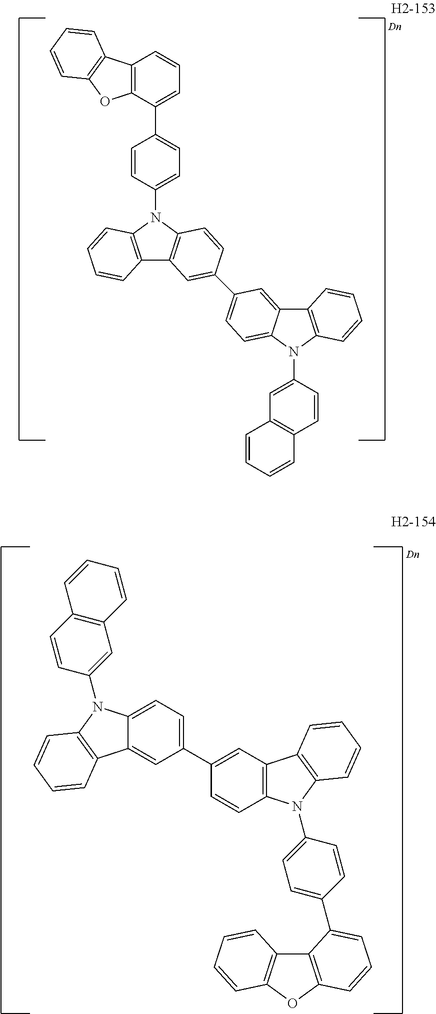

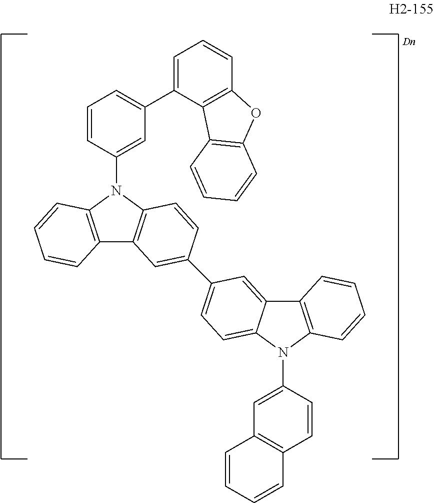

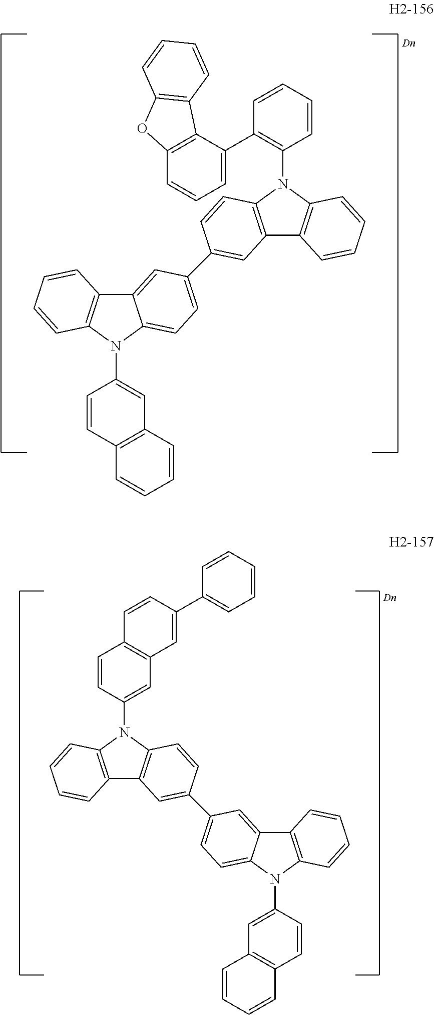

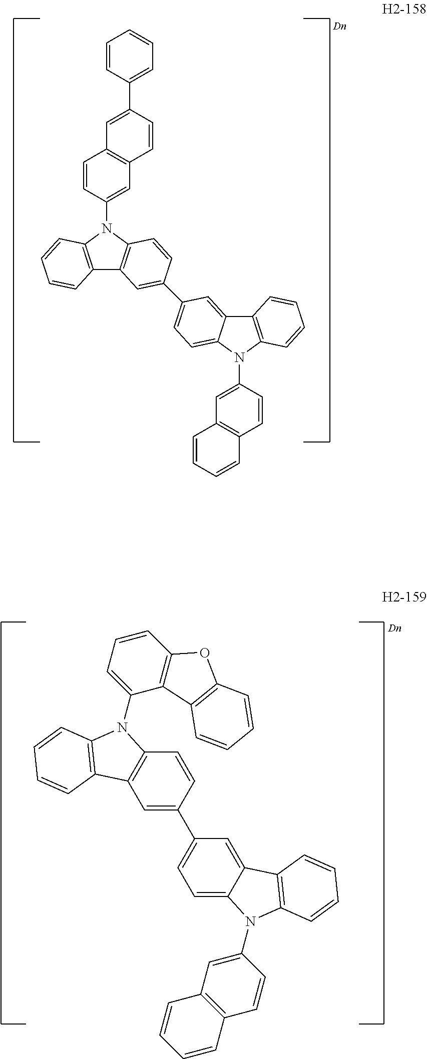

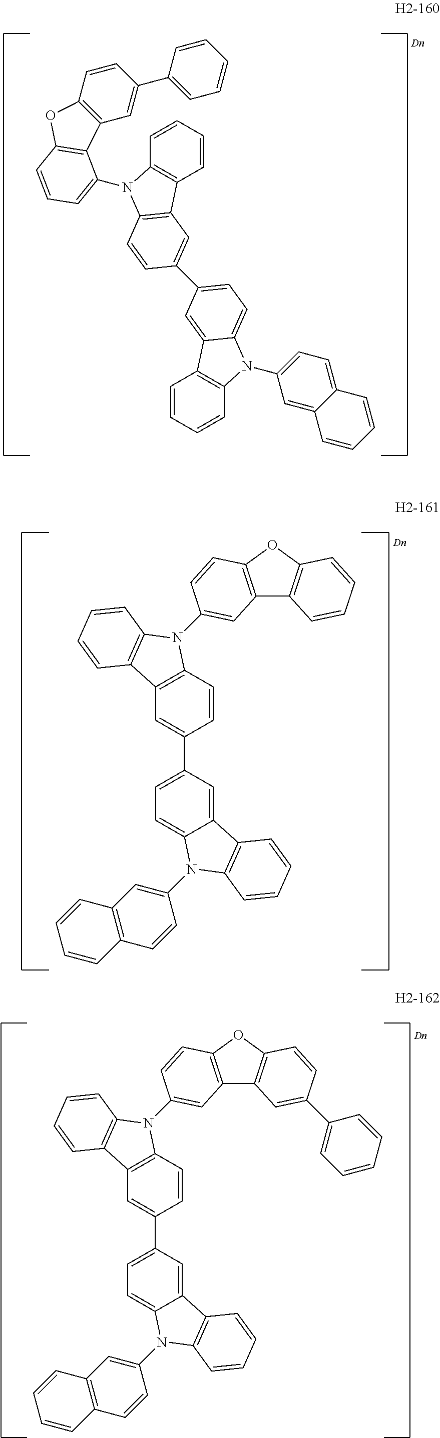

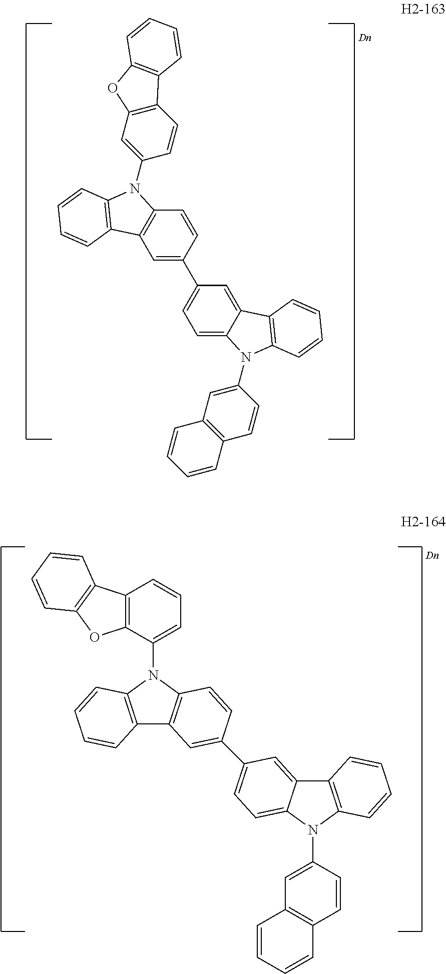

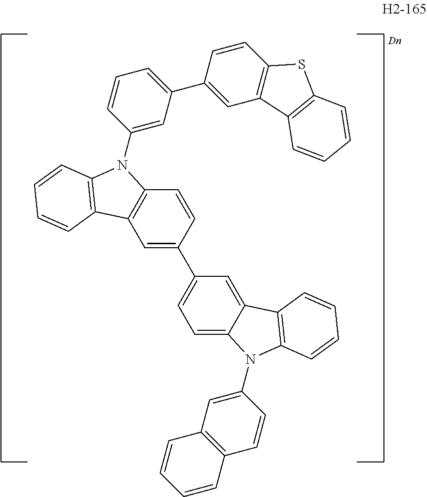

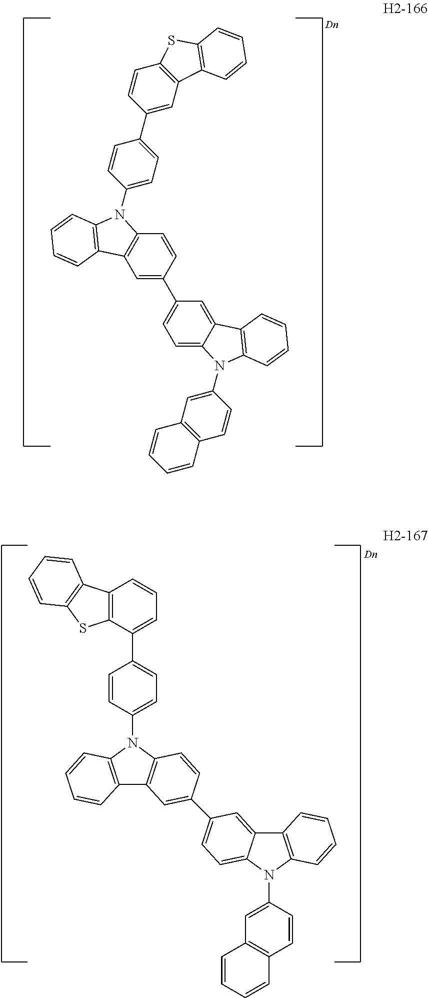

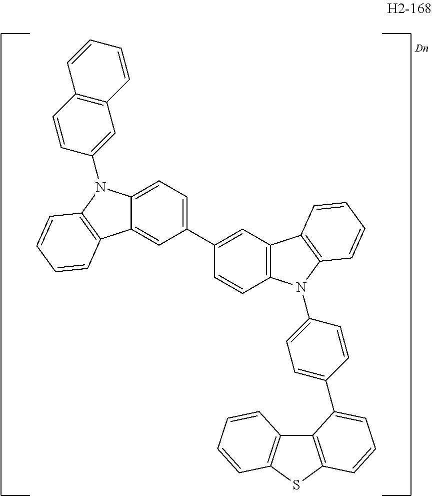

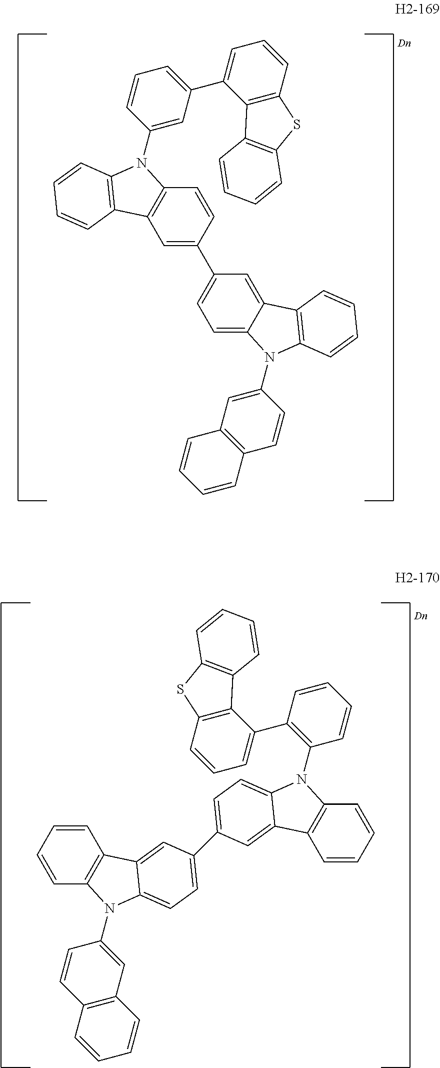

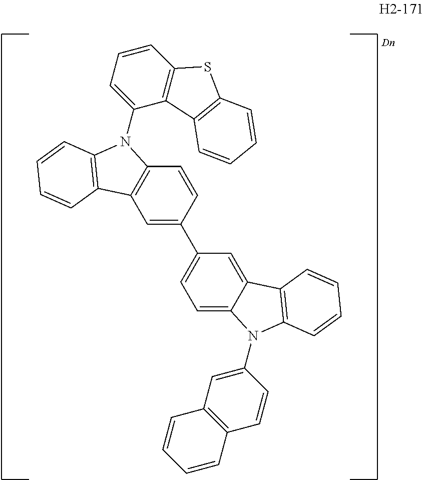

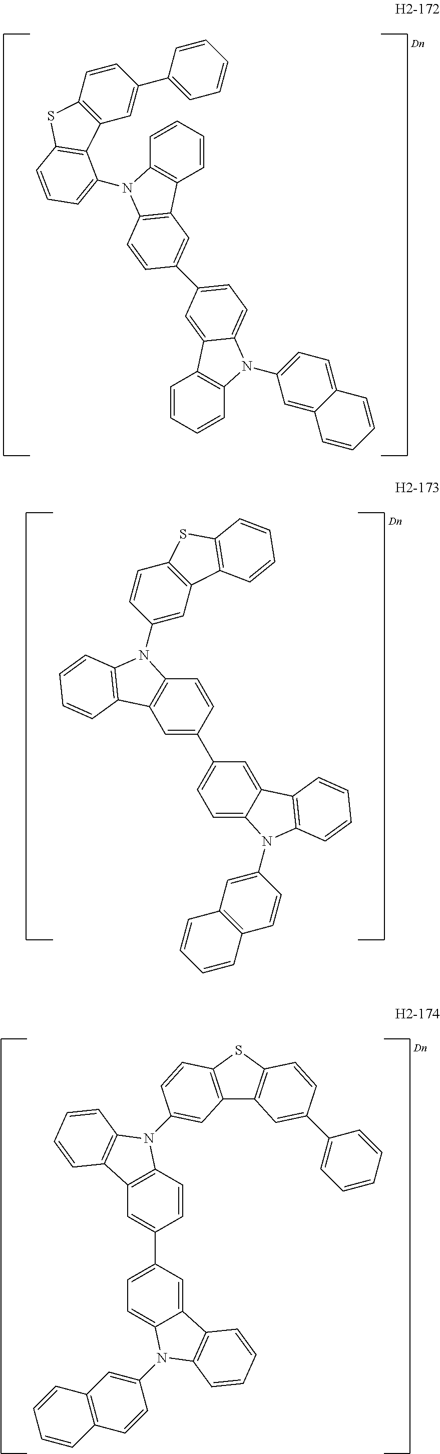

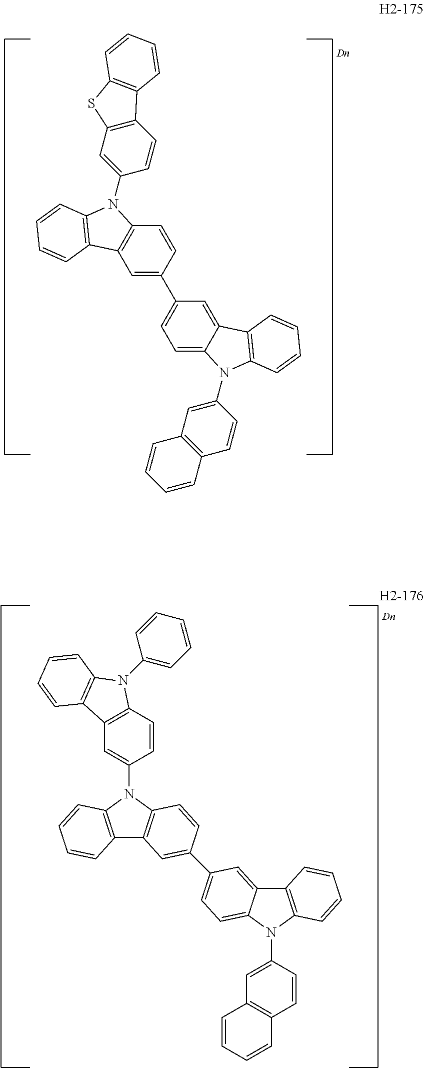

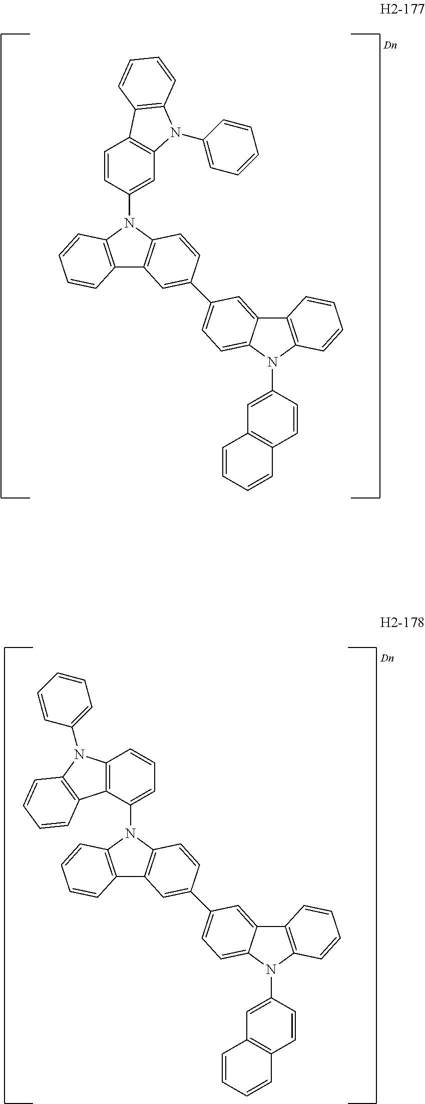

The compound represented by formula 1′ may be any one selected from the group consisting of compounds H1-1 to H1-260 above, but is not limited thereto.

The compound represented by formula 2′ may be any one selected from the group consisting of compounds H2-34 to H2-178 above, but is not limited thereto.

In the compounds, Dₙ represents that n number of hydrogens are replaced with deuterium, and n represents an integer of 1 to 50. According to one embodiment of the present disclosure, n represents an integer of 4 or more, preferably an integer of 5 or more, more preferably an integer of 8 or more, and even more preferably an integer of 11 or more. When being deuterated to the number of the lower limit or more, the bond dissociation energy according to deuteration may increase to enhance the stability of the compound, and improved lifetime properties can be exhibited by using the compound in an organic electroluminescent device.

According to one embodiment of the present disclosure, at least one of compounds H1-1 to H1-260 and H1′-1 to H1′-265, and at least one of compounds H2-1 to H2-178 may be used in an organic electroluminescent device. According to another embodiment of the present disclosure, the combination of at least one of compounds H1-1 to H1-260 and H1′-1 to H1′-265, and at least one of compounds H2-1 to H2-178, or the combination of at least one of compounds H1-1 to H1-260 and H1′-1 to H1′-265, and at least one of compounds H2-34 to H2-178 may be used in an organic electroluminescent device.

The compound represented by formula 1 or 1′ according to the present disclosure may be produced by a synthetic method known to one skilled in the art, and for example, by referring to Korean Patent Application Laid-Open No. 2011-0122051 (published on Nov. 9, 2011), Korean Patent No. 1396171 (published on May 27, 2014), etc., or by referring to the following reaction schemes 1 and 1′, but is not limited thereto.

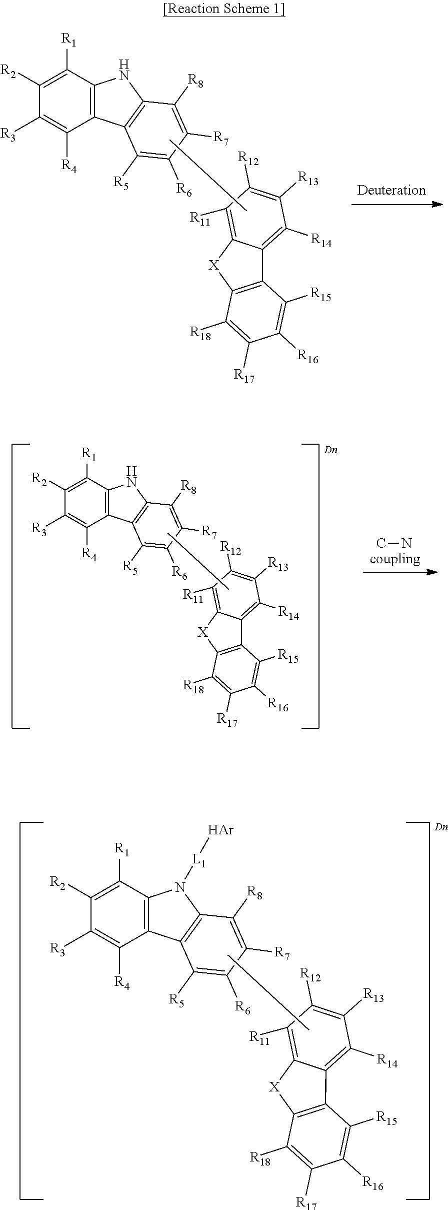

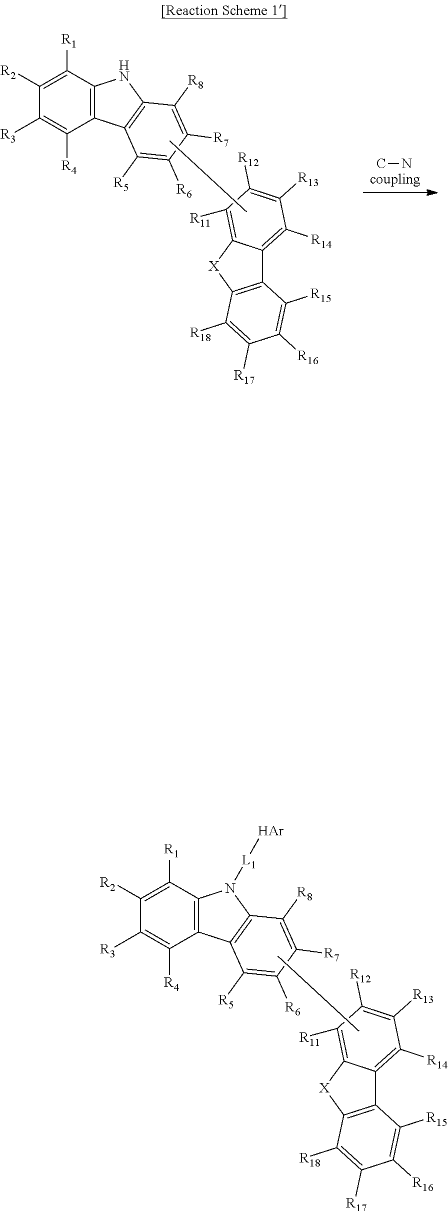

In reaction schemes 1 and 1′, L₁, HAr, X, R₁ to R₈, and R₁₁ to R₁₈ are as defined in formula 1; and Dn represents that n of hydrogens are replaced with deuterium.

The compound represented by formula 2 or 2′ according to the present disclosure may be produced by a synthetic method known to one skilled in the art, and for example, by referring to Korean Patent Application Laid-Open No. 2013-0018724 (published on Feb. 25, 2013). Korean Patent Application Laid-Open No. 2014-0049227 (published on Apr. 25, 2014), etc., or by referring to the following reaction scheme 2, but is not limited thereto.

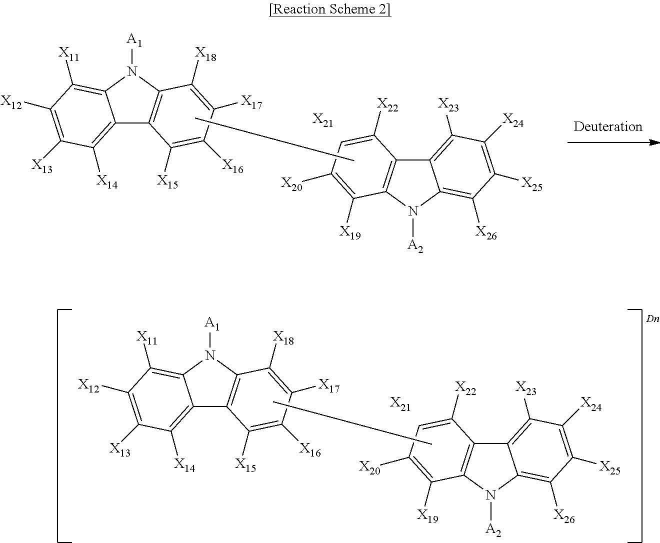

In reaction scheme 2, A₁, A₂, and X₁₁ to X₂₆ are as defined in formula 2, and Dn represents that n hydrogens are replaced with deuterium.

Although illustrative synthesis examples of the compound represented by formula 1, 1′, 2 or 2′ of the present disclosure are described above, one skilled in the art will be able to readily understand that all of them are based on a Buchwald-Hartwig cross-coupling reaction, an N-arylation reaction, a H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN₁ substitution reaction, an SN₂ substitution reaction, a Phosphine-mediated reductive cyclization reaction, etc., and the reactions above proceed even when substituents, which are defined in formulas 1, 1′, 2 and 2′ but are not specified in the specific synthesis examples, are bonded.

The deuterated compound of formulas 1, 1′, 2 and 2′ may be prepared in a similar manner by using deuterated precursor materials, or more generally may be prepared by treating the non-deuterated compound with a deuterated solvent or D6-benzene in the presence of an H/D exchange catalyst such as a Lewis acid, e.g., aluminum trichloride or ethyl aluminum chloride. In addition, the degree of deuteration can be controlled by changing the reaction conditions such as the reaction temperature. For example, the number of deuterium in formulas 1, 1′, 2 and 2′ can be controlled by adjusting the reaction temperature and time, the equivalent of the acid, etc.

The organic electroluminescent device according to the present disclosure may comprise an anode, a cathode, and at least one organic layer between the anode and cathode, in which the organic layer may comprise a plurality of organic electroluminescent materials, including the compound represented by formula 1 or 1′ as the first organic electroluminescent material, and the compound represented by formula 2 or 2′ as the second organic electroluminescent material. According to one embodiment of the present disclosure, the organic electroluminescent device according to the present disclosure may comprise an anode, a cathode, and at least one light-emitting layer between the anode and cathode, in which the light-emitting layer(s) may comprise the compound represented by formula 1 or 1′, and the compound represented by formula 2 or 2′.

The light-emitting layer includes a host(s) and a dopant, in which the host(s) includes a plurality of host materials, and the compound represented by formula 1 or 1′ may be included as the first host compound of the plurality of host materials, and the compound represented by formula 2 or 2′ may be included as the second host compound of the plurality of host materials. The weight ratio of the first host compound and the second host compound is about 1:99 to about 99:1, preferably about 10:90 to about 90:10, more preferably about 30:70 to about 70:30, even more preferably about 40:60 to about 60:40, and still more preferably about 50:50.

In the present disclosure, the light-emitting layer is a layer from which light is emitted, and may be a single layer or a mufti-layer of which two or more layers are stacked. All of the first and second host materials may be included in one layer, or the first host material and the second host material may be included in respective different light-emitting layers. According to one embodiment of the present disclosure, the doping concentration of the dopant compound with respect to the host compound(s) in the light-emitting layer may be less than 20 wt %.

The organic electroluminescent device of the present disclosure may further comprise at least one layer selected from a hole injection layer, a hole transport layer, a hole auxiliary layer, a light-emitting auxiliary layer, an electron transport layer, an electron injection layer, an interlayer, an electron buffer layer, a hole blocking layer, and an electron blocking layer. According to one embodiment of the present disclosure, the organic electroluminescent device of the present disclosure may further comprise an amine-based compound besides the plurality of host materials of the present disclosure as at least one of a hole injection material, a hole transport material, a hole auxiliary material, a light-emitting material, a light-emitting auxiliary material, and an electron blocking material. Further, according to one embodiment of the present disclosure, the organic electroluminescent device of the present disclosure may further comprise an azine-based compound besides the plurality of host materials of the present disclosure as at least one of an electron transport material, an electron injection material, an electron buffer material, and a hole blocking material.

The plurality of host materials according to the present disclosure may be used as a light-emitting material(s) for a white organic light-emitting device. The white organic light-emitting device has been suggested in various structures such as a side-by-side structure or a stacking structure depending on the arrangement of R (Red), G (Green) or YG (Yellow Green), and B (Blue) light-emitting parts, or color conversion material (CCM) method, etc. In addition, the plurality of host materials according to the present disclosure may also be used in an organic electroluminescent device comprising a quantum dot (QD).

A hole injection layer, a hole transport layer, or an electron blocking layer, or a combination thereof may be used between the anode and the light-emitting layer. The hole injection layer may be multilayers in order to lower the hole injection barrier (or hole injection voltage) from the anode to the hole transport layer or the electron blocking layer, wherein each of the multilayers may use two compounds simultaneously. In addition, the hole injection layer may be further doped with a p-dopant. The electron blocking layer may be placed between the hole transport layer (or hole injection layer) and the light-emitting layer, and may block overflowing electrons from the light-emitting layer and confine the excitons in the light-emitting layer to prevent light leakage. The hole transport layer or the electron blocking layer may be multilayers, wherein each of the multilayers may use a plurality of compounds.

An electron buffer layer, a hole blocking layer, an electron transport layer, or an electron injection layer, or a combination thereof can be used between the light-emitting layer and the cathode. The electron buffer layer may be multilayers in order to control the injection of the electron and improve the interfacial properties between the light-emitting layer and the electron injection layer, wherein each of the multilayers may use two compounds simultaneously. The hole blocking layer or the electron transport layer may also be multilayers, wherein each of the multilayers may use a plurality of compounds. In addition, the electron injection layer may be doped with an n-dopant.

The dopant comprised in the organic electroluminescent device of the present disclosure may be at least one phosphorescent or fluorescent dopant, and preferably a phosphorescent dopant. The phosphorescent dopant material applied to the organic electroluminescent device of the present disclosure is not particularly limited, but may be preferably selected from the metallated complex compounds of iridium (Ir), osmium (Os), copper (Cu), and platinum (Pt), more preferably selected from ortho-metallated complex compounds of iridium (Ir), osmium (Os), copper (Cu), and platinum (Pt), and even more preferably ortho-metallated iridium complex compounds.

The dopant comprised in the organic electroluminescent device of the present disclosure may comprise a compound represented by the following formula 101, but is not limited thereto.

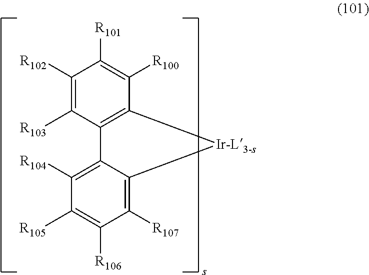

In formula 101, L′ is selected from the following structures 1 to 3:

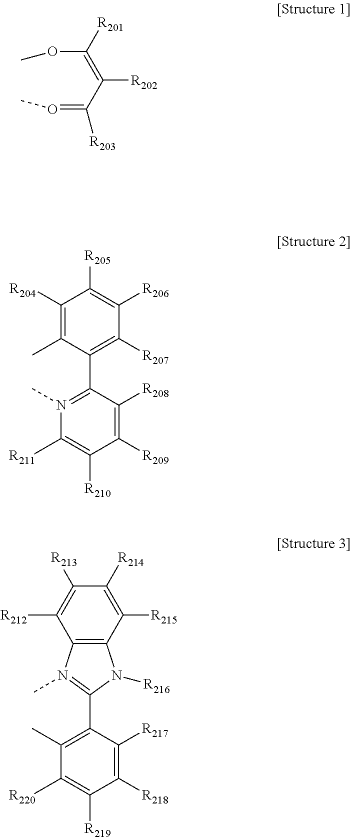

R₁₀₀ to R₁₀₃, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent to form a ring(s), e.g., a substituted or unsubstituted, quinoline, isoquinoline, benzofuropyridine, benzothienopyridine, indenopyridine, benzofuroquinoline, benzothienoquinoline, or indenoquinoline ring, together with pyridine;

R₁₀₄ to R₁₀₇, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent to form a ring(s), e.g., a substituted or unsubstituted, naphthalene, fluorene, dibenzothiophene, dibenzofuran, indenopyridine, benzofuropyridine or benzothienopyridine ring, together with benzene;

R₂₀₁ to R₂₂₀, each independently, represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent to form a ring(s); and

s represents an integer of 1 to 3.

The specific examples of the dopant compound are as follows, but are not limited thereto.

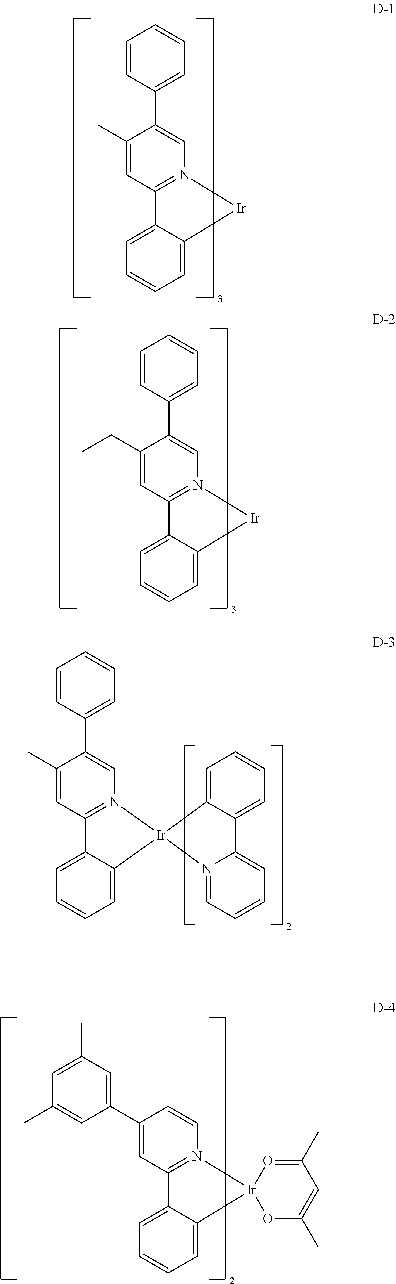

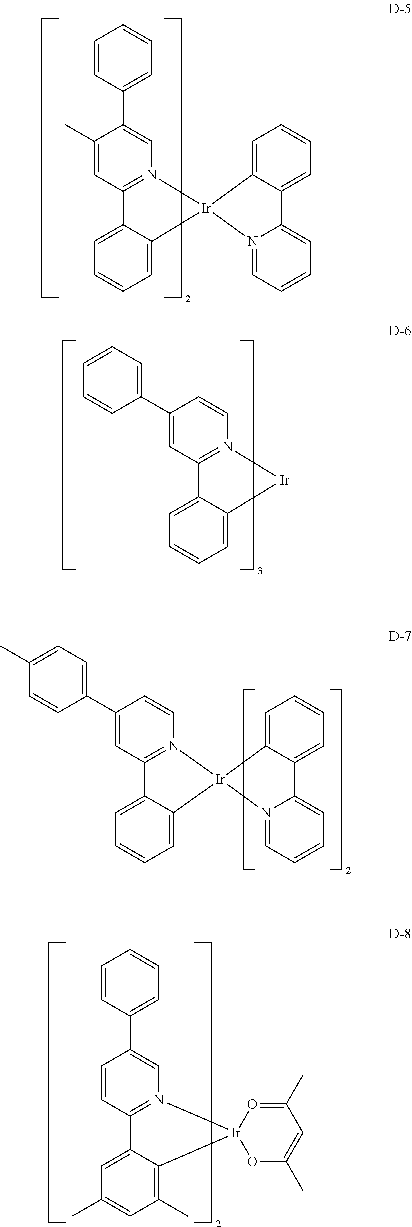

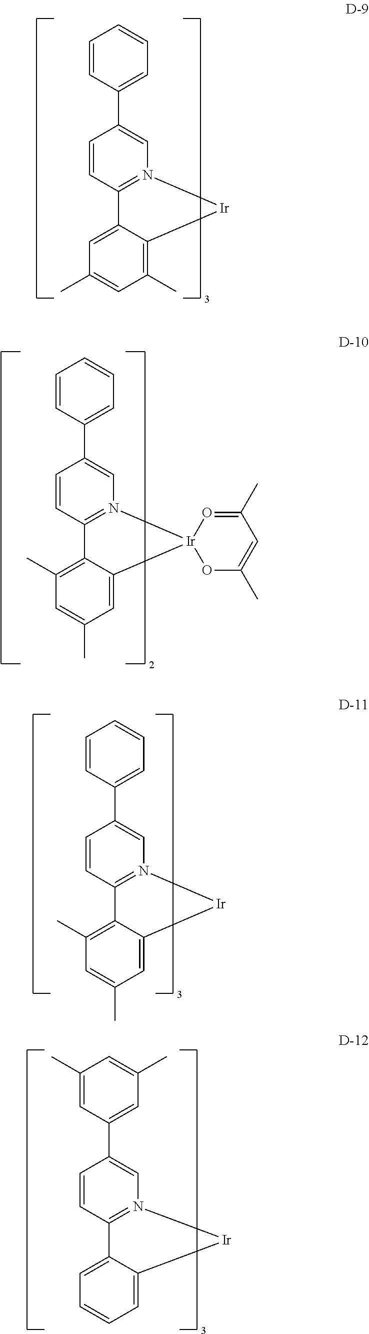

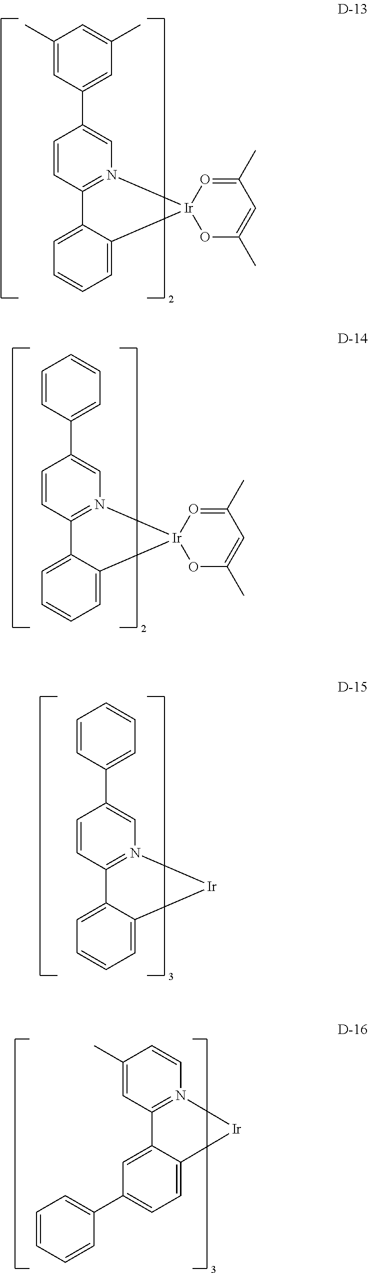

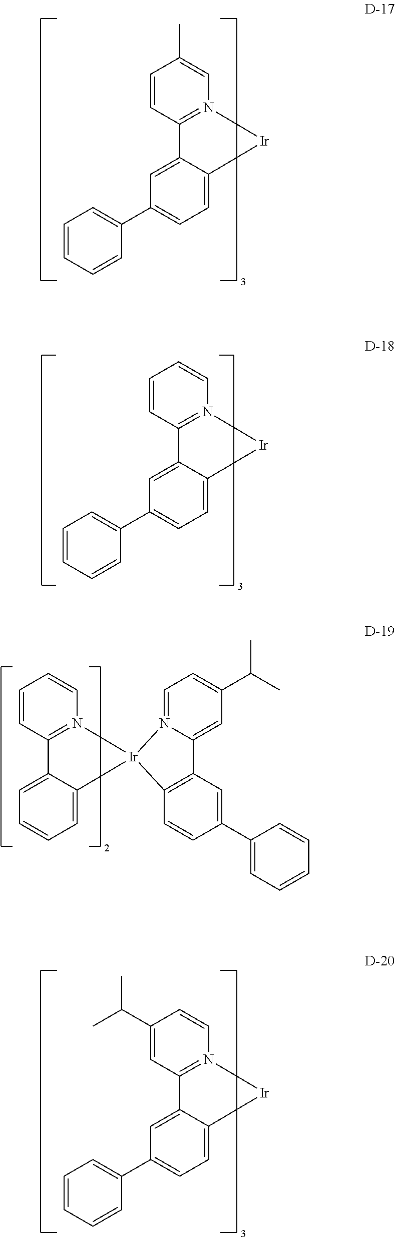

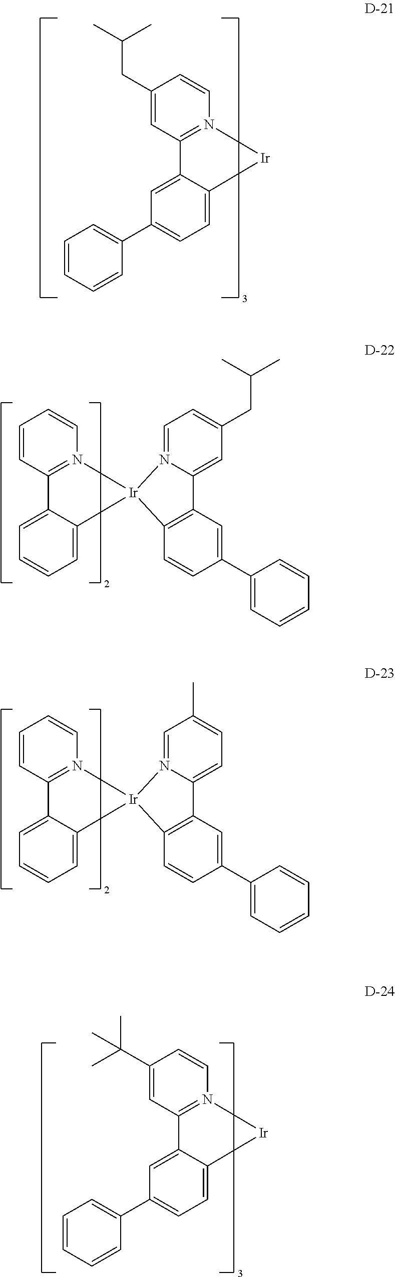

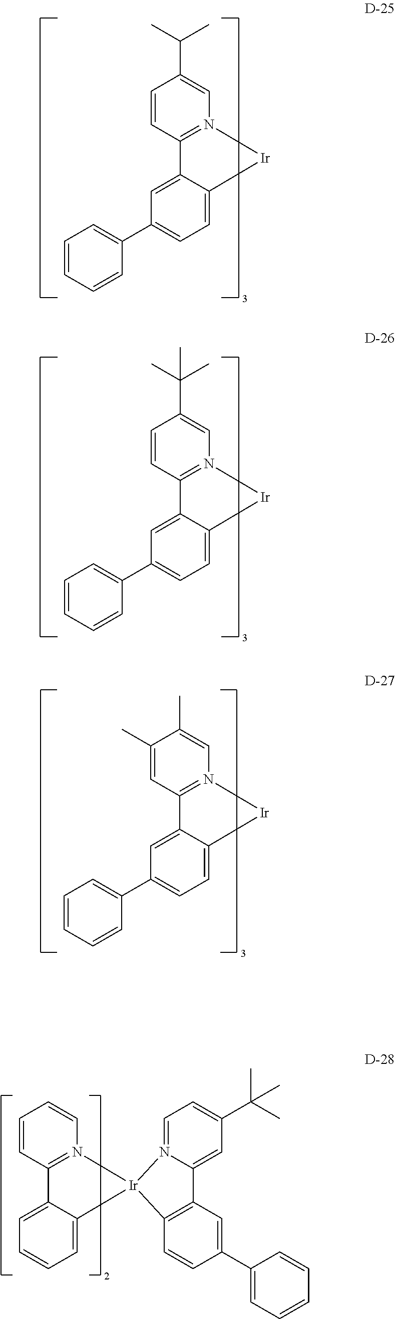

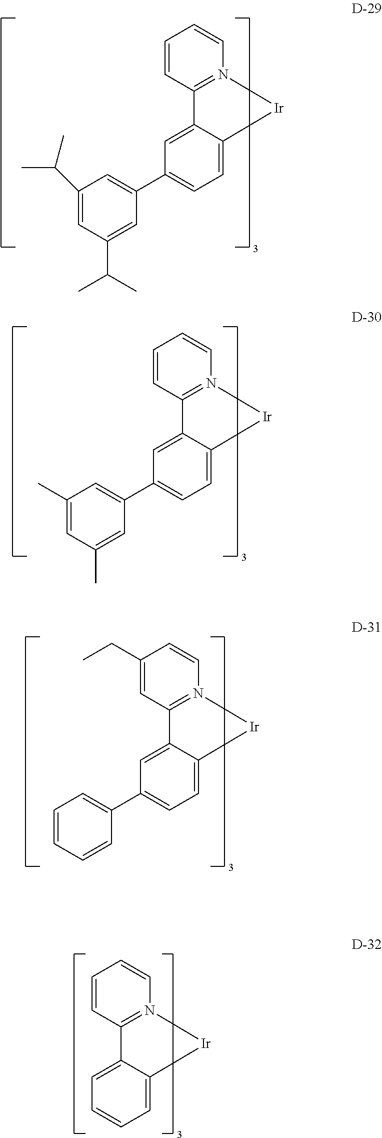

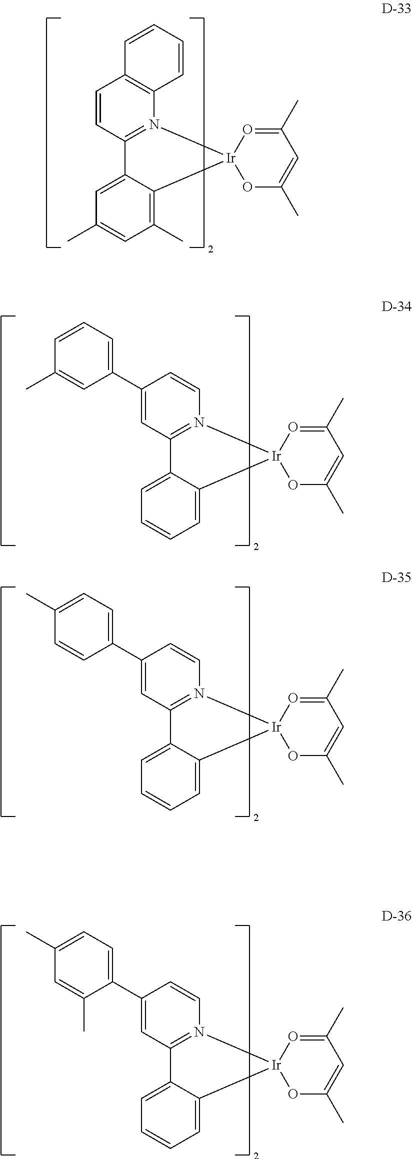

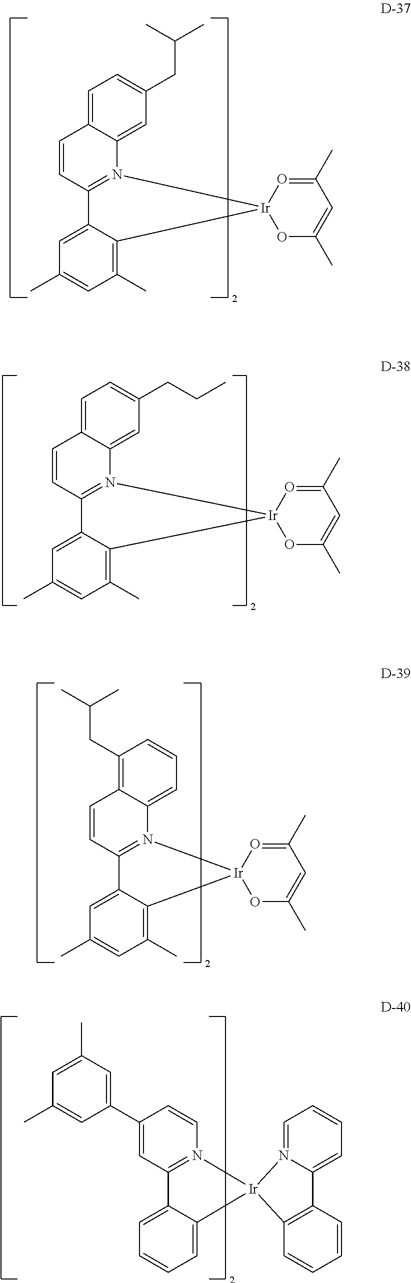

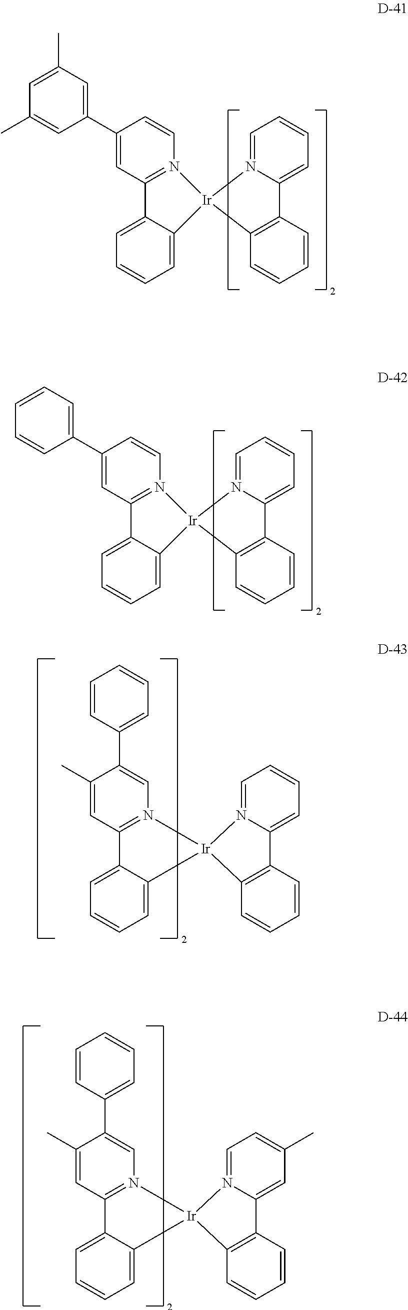

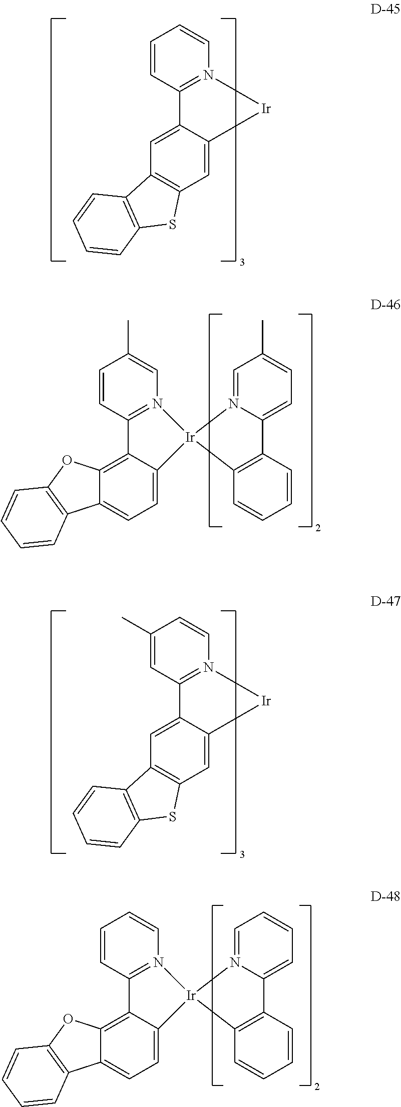

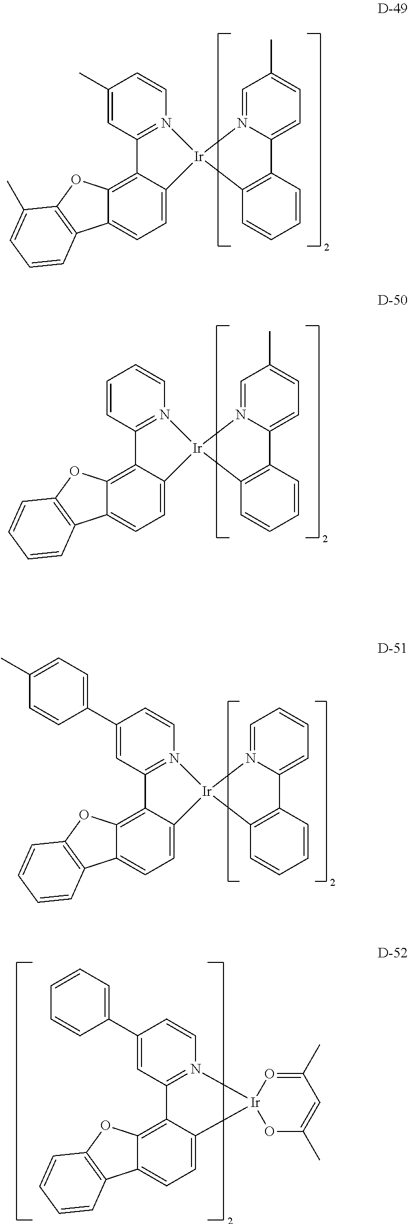

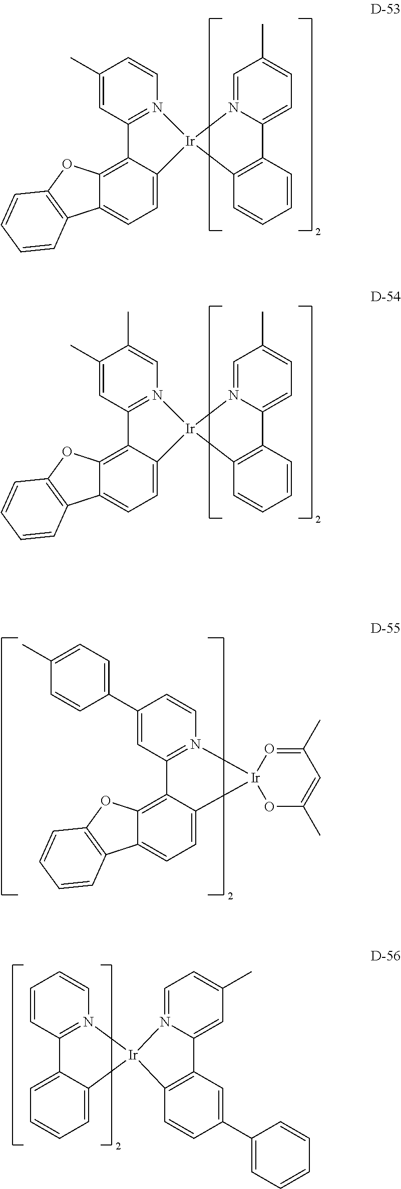

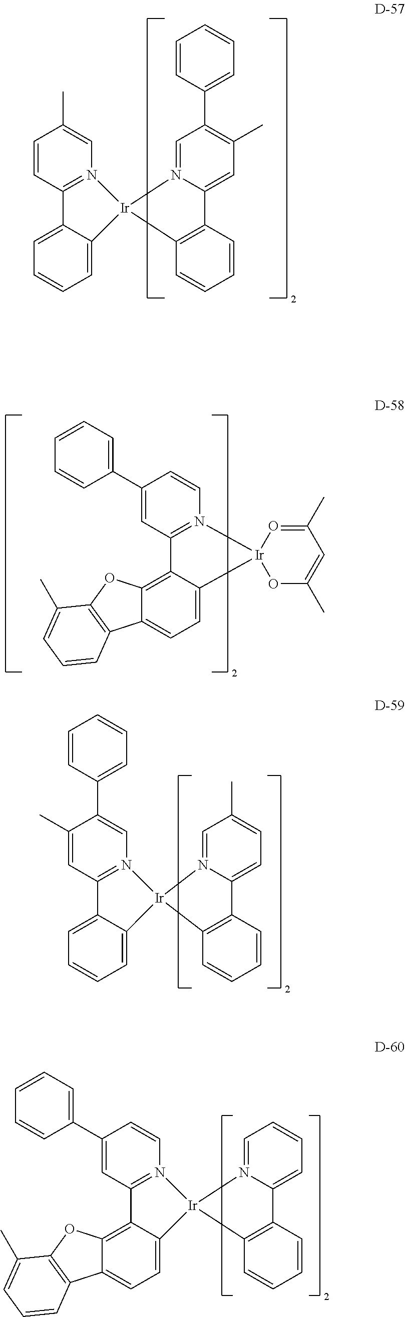

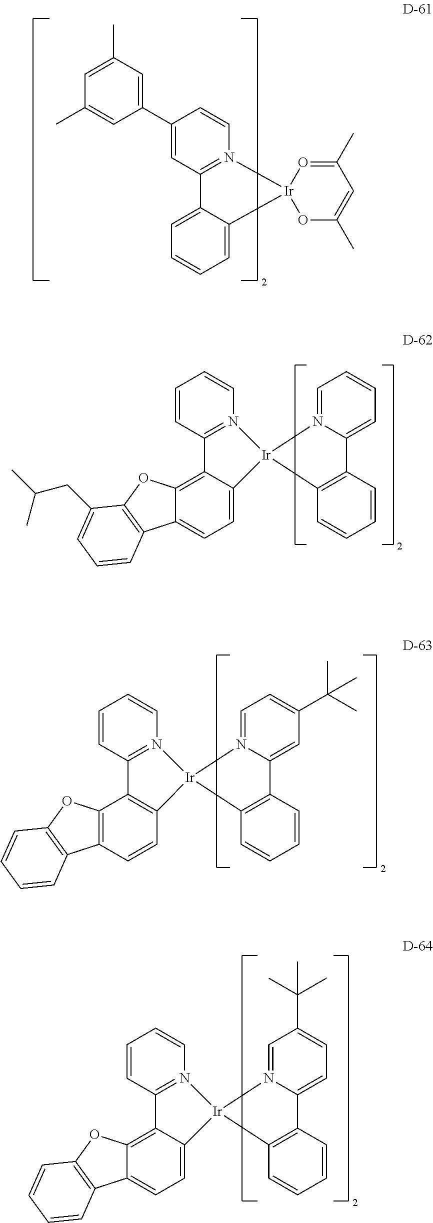

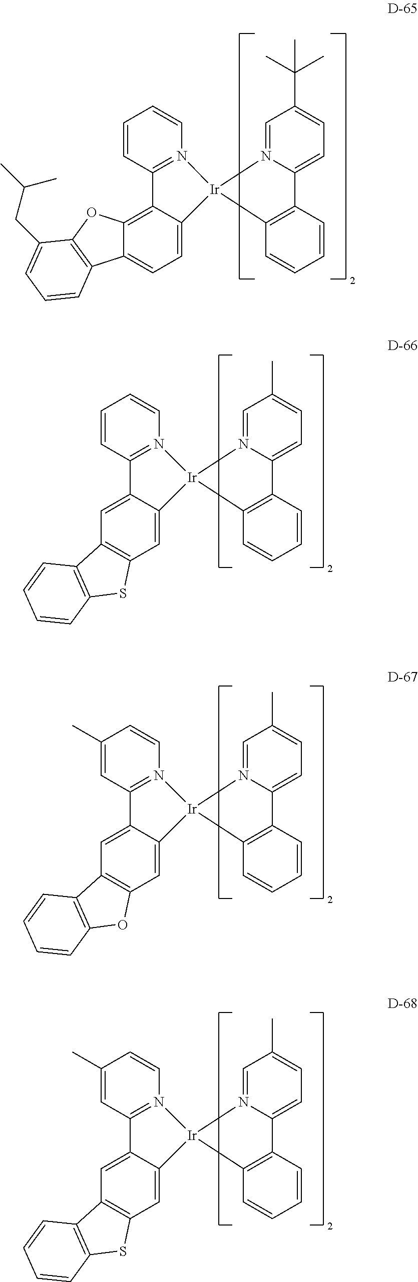

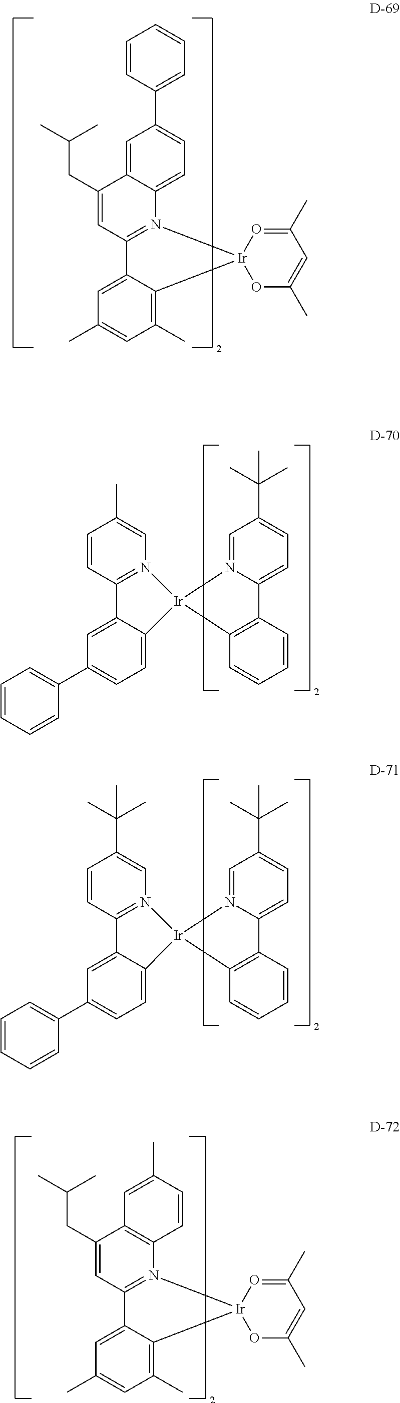

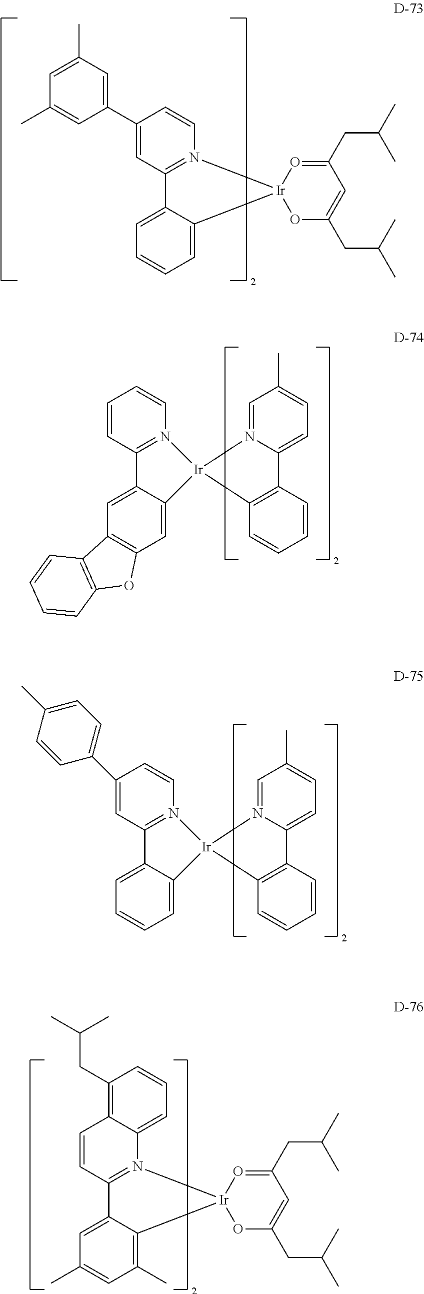

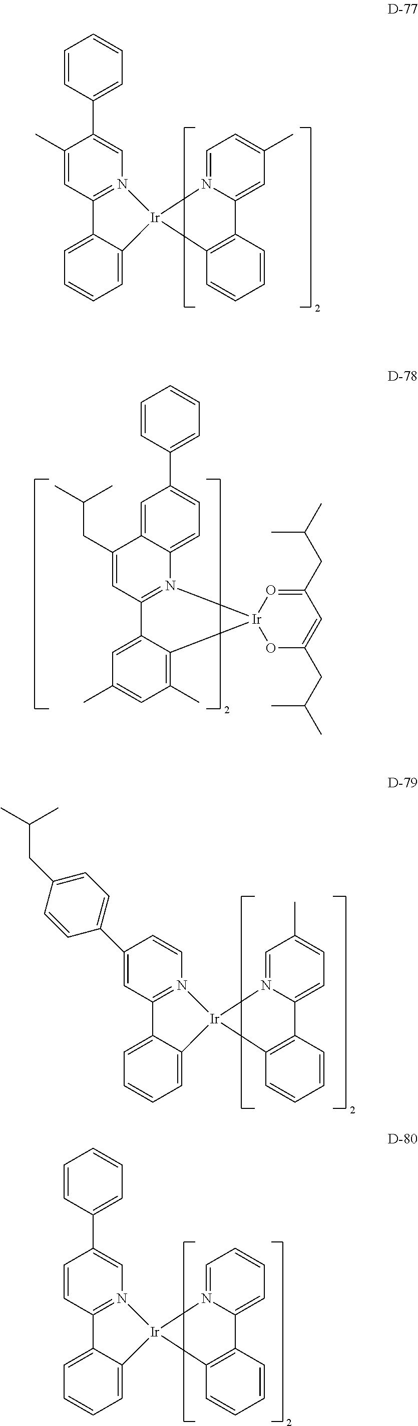

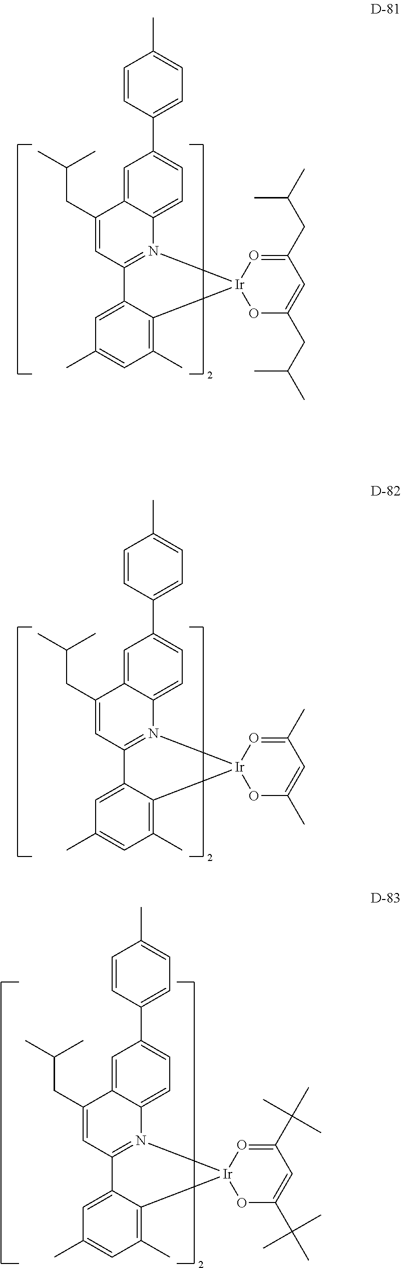

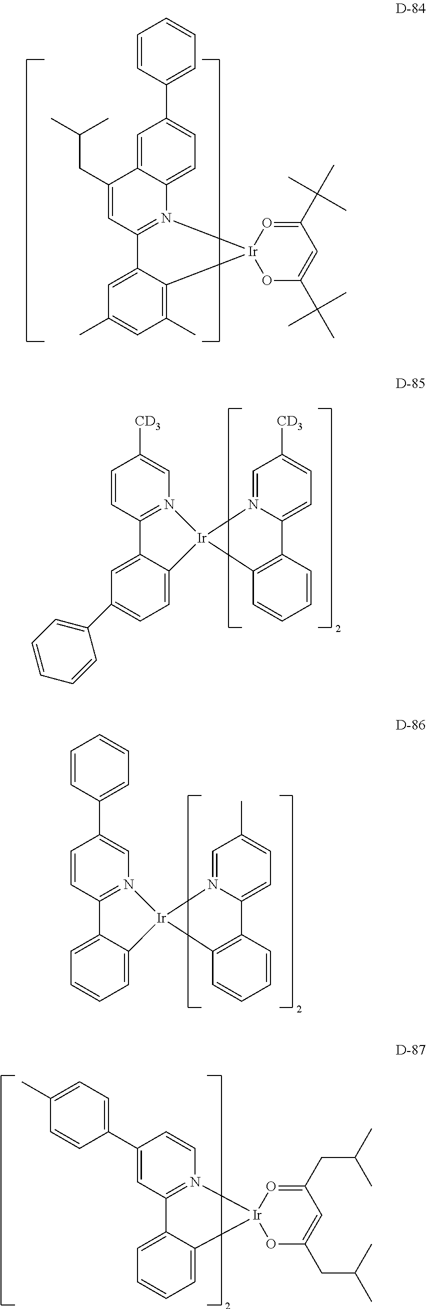

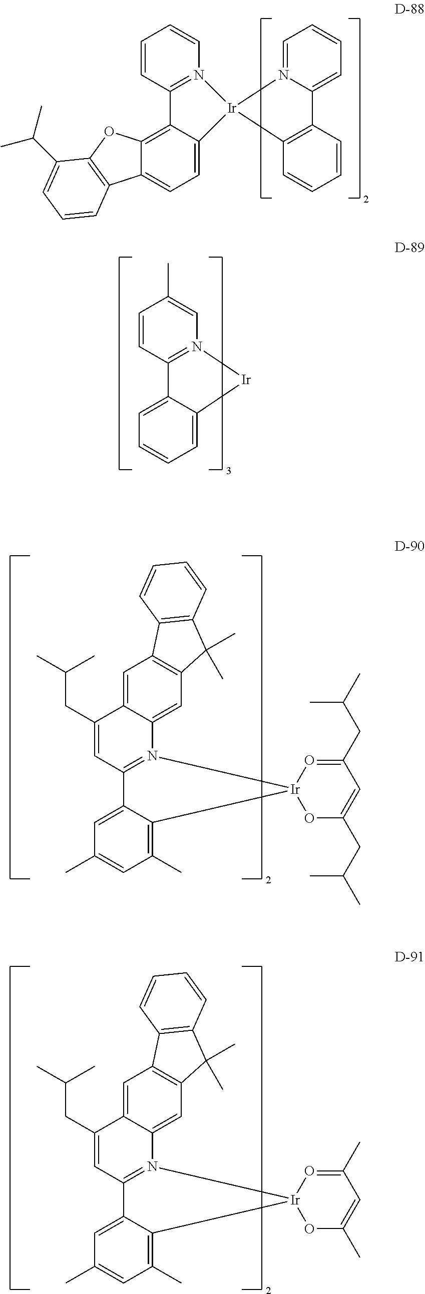

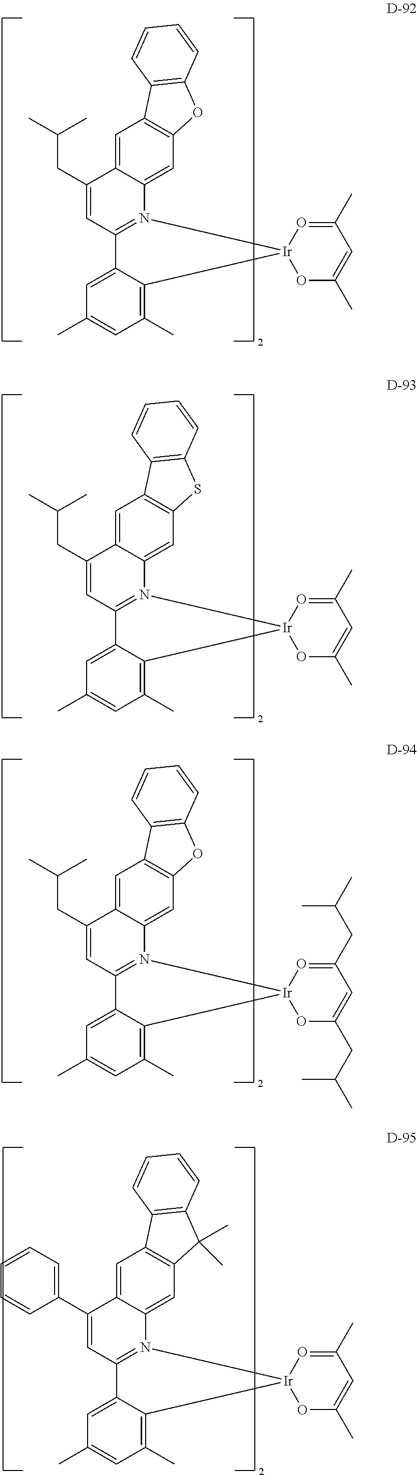

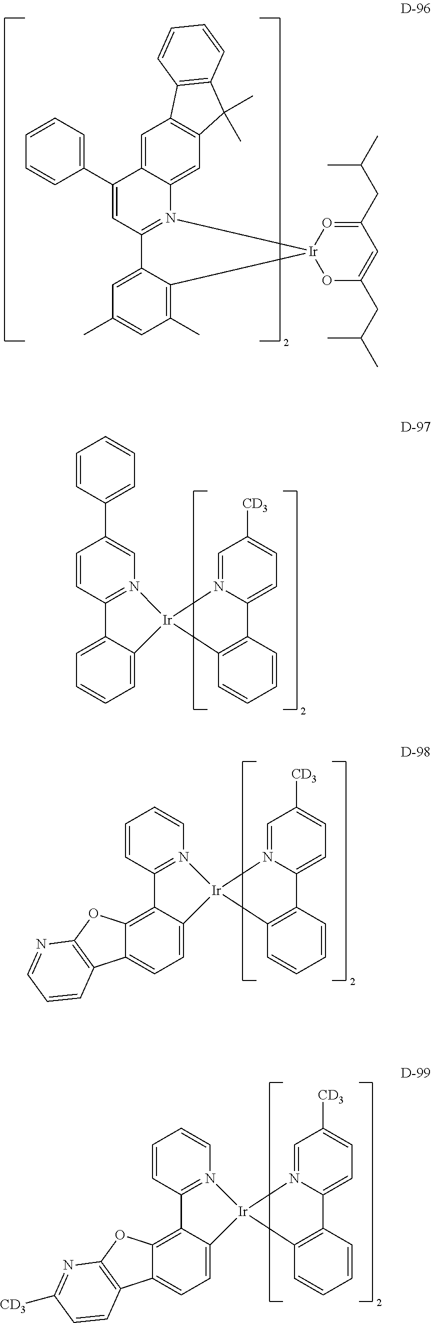

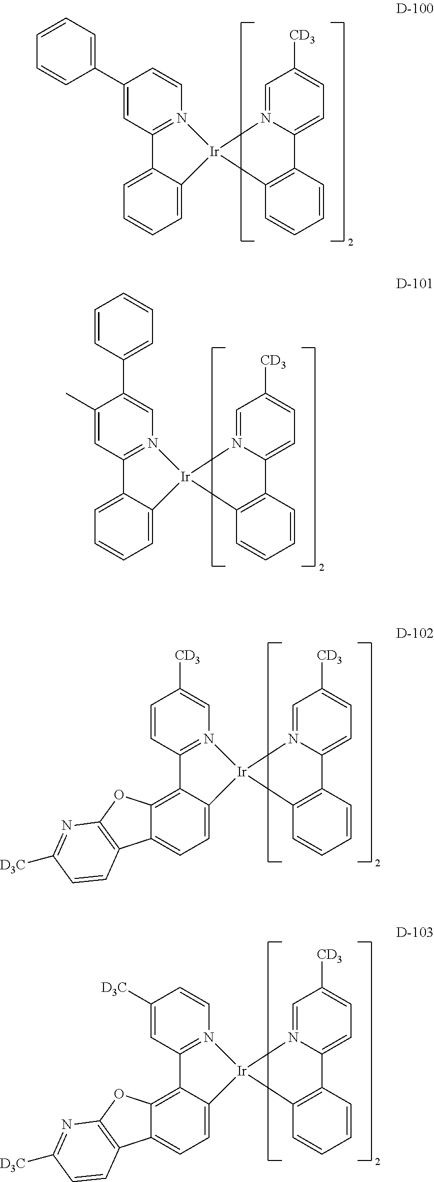

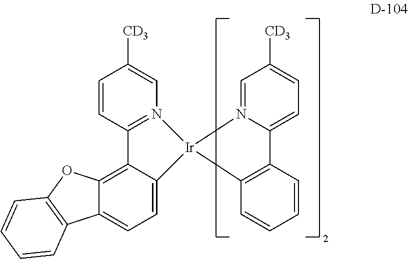

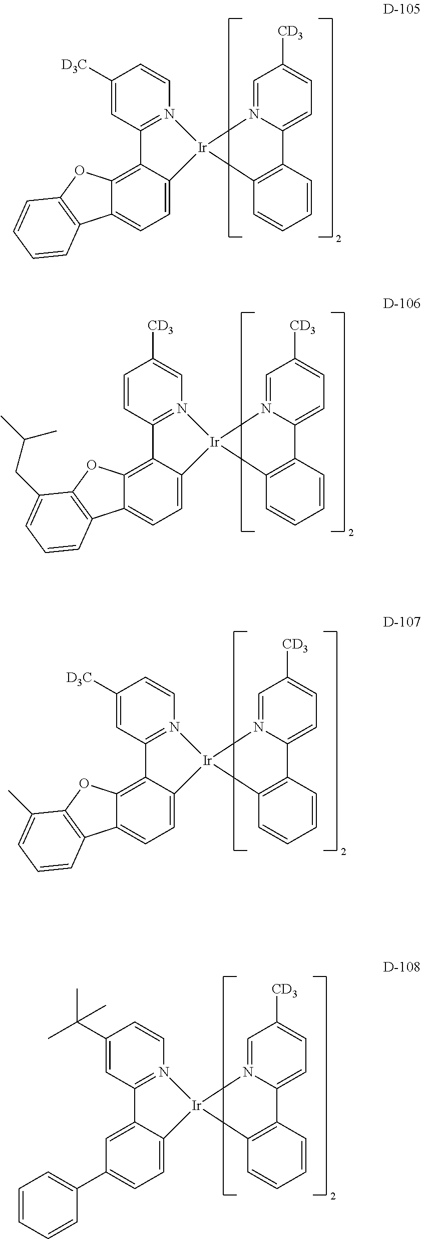

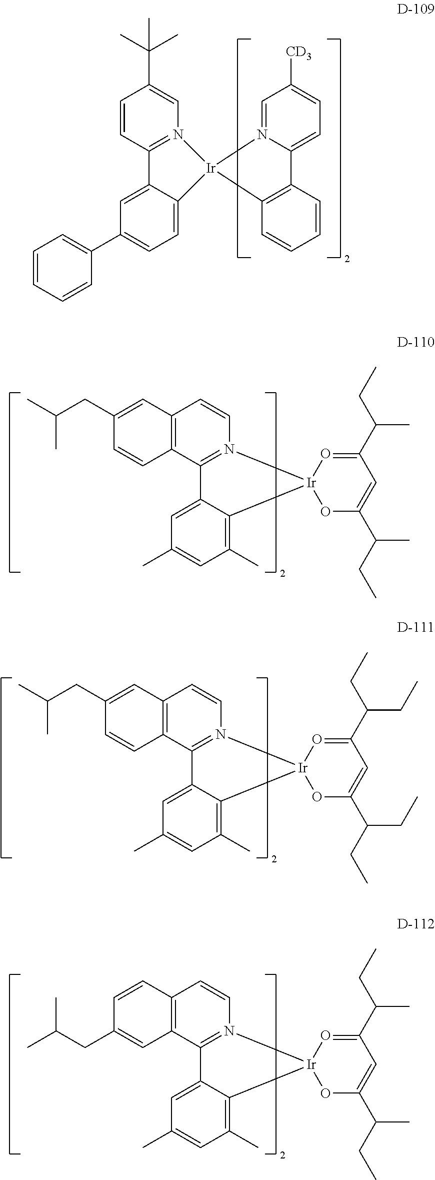

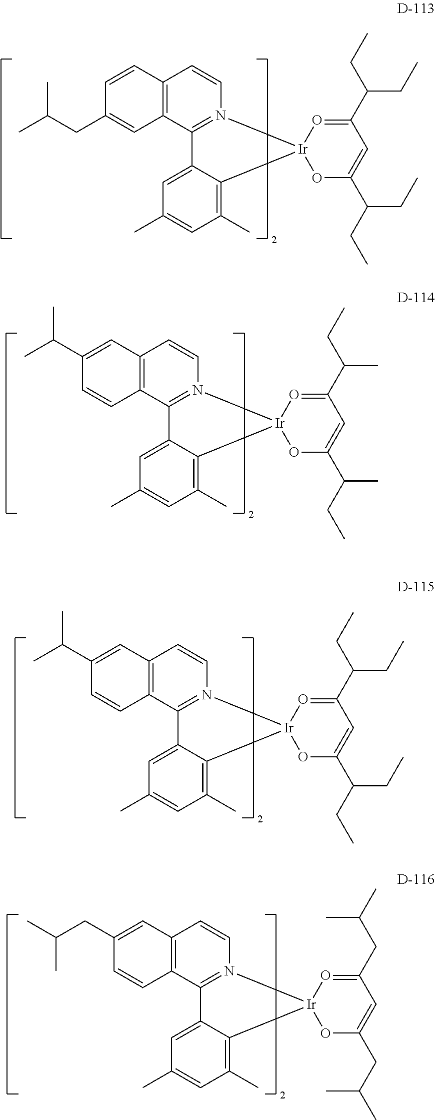

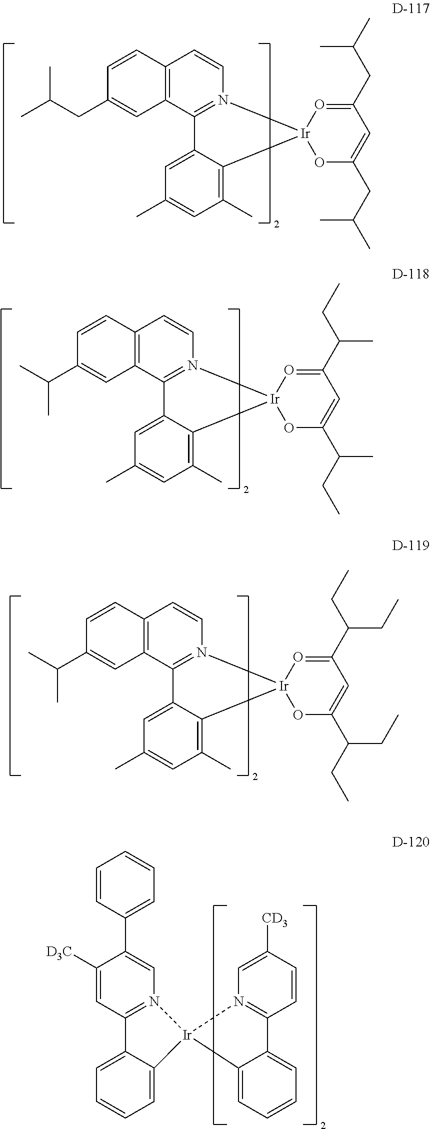

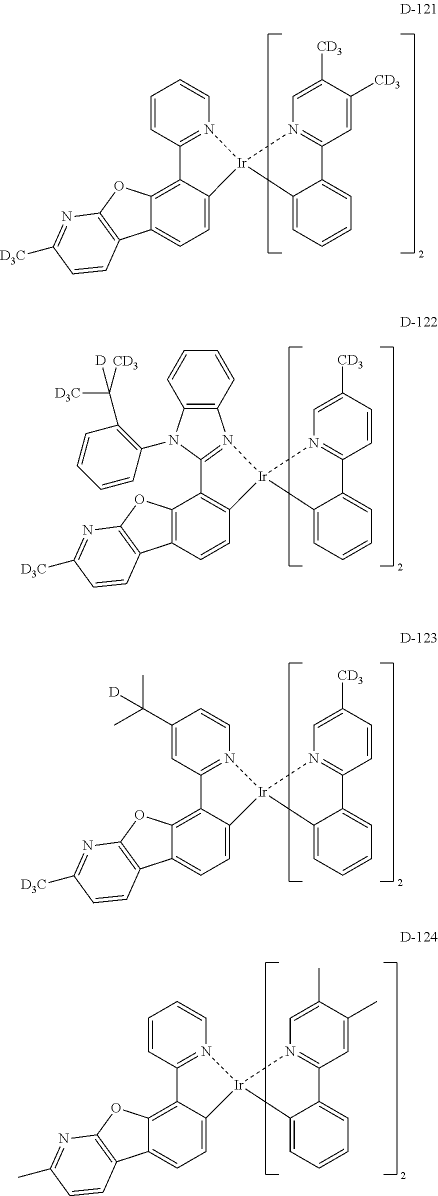

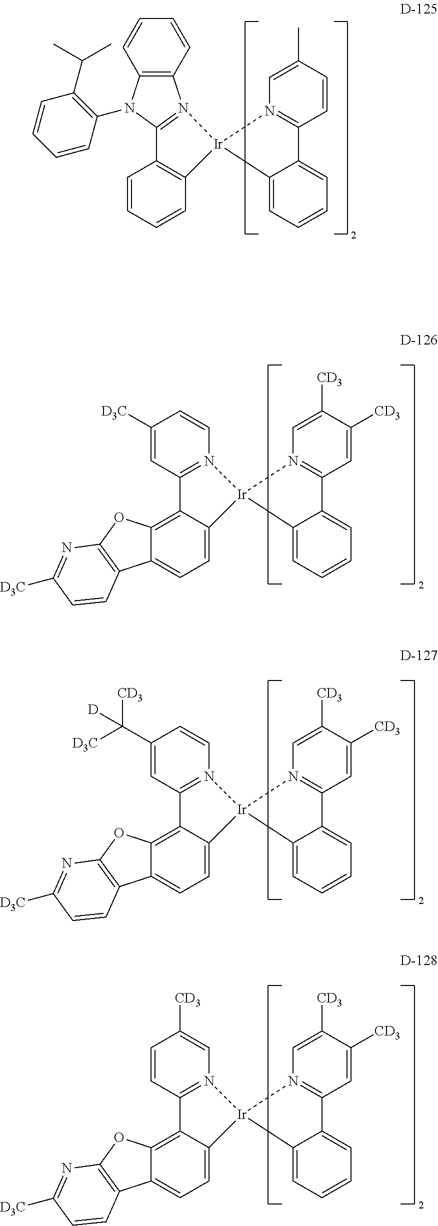

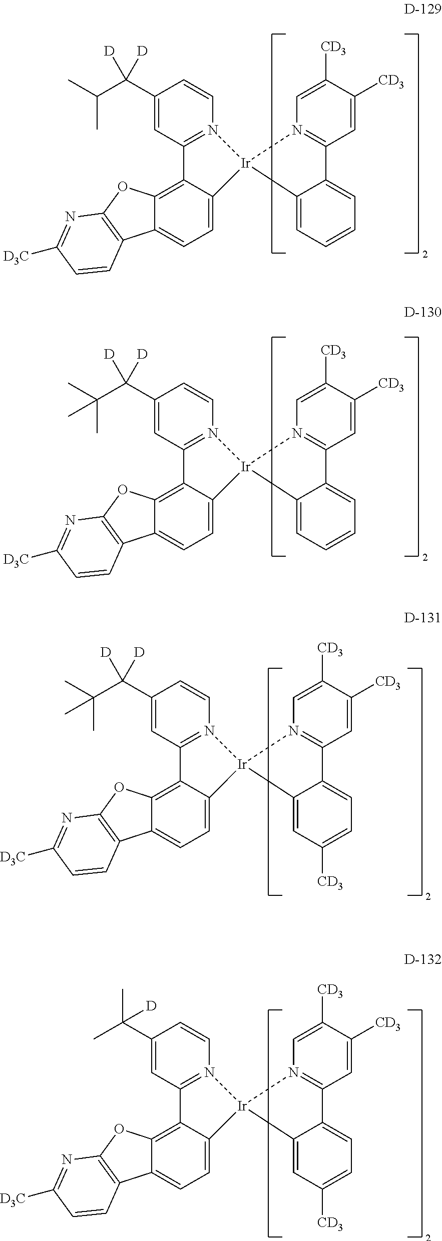

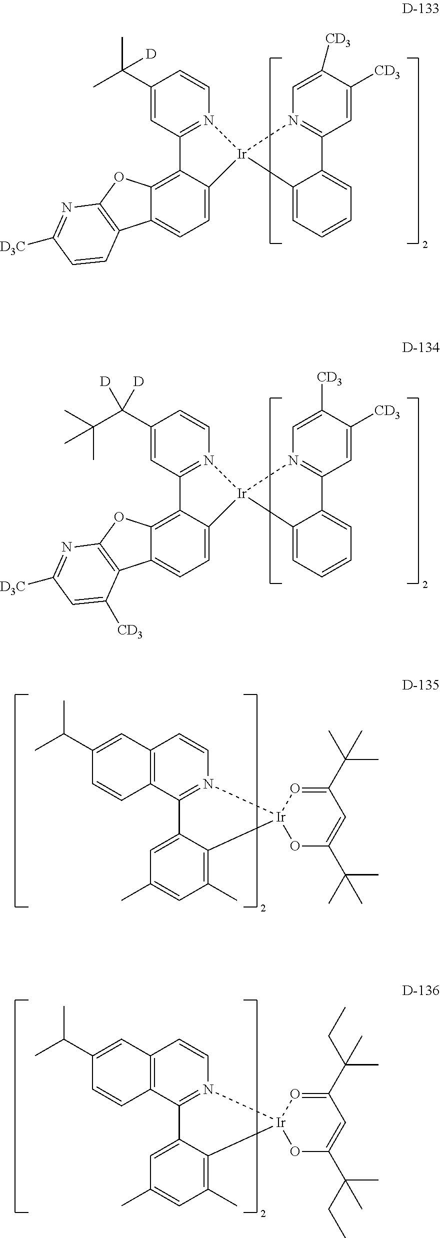

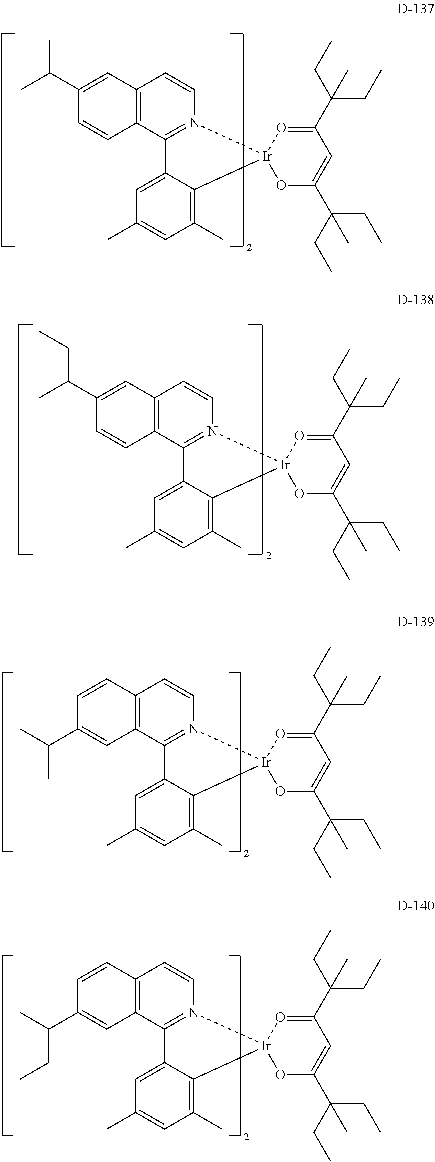

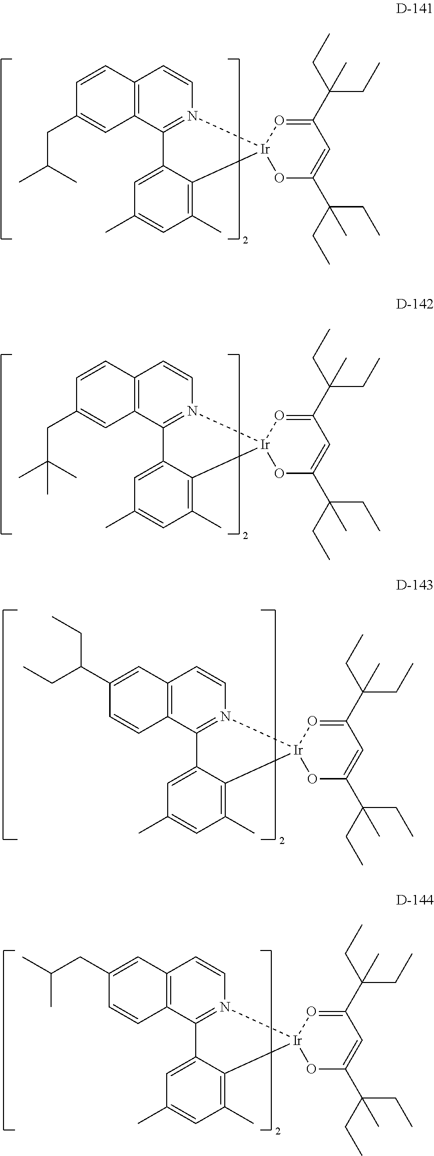

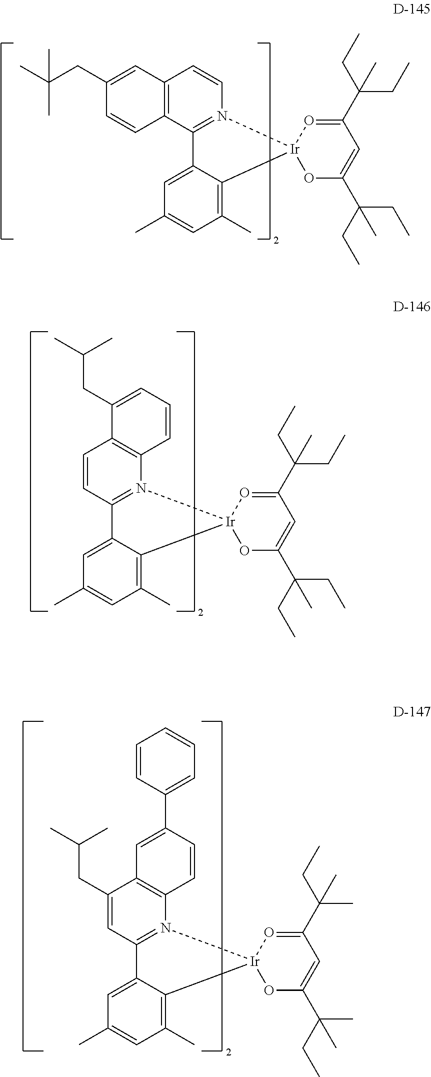

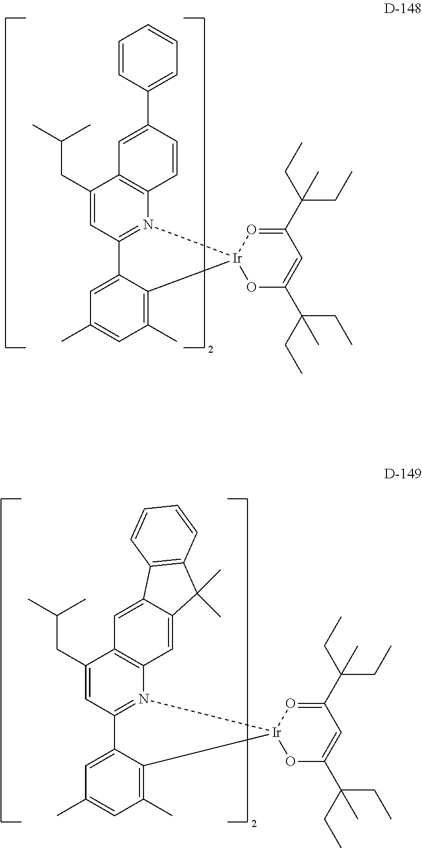

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film can be formed by dissolving or diffusing materials forming each layer into any suitable solvent such as ethanol, chloroform, tetrahydrofuran, dioxane, etc. The solvent can be any solvent where the materials forming each layer can be dissolved or diffused, and where there are no problems in film-formation capability.

The first and the second host compounds of the present disclosure may be film-formed by the above-listed methods, commonly by a co-evaporation process or a mixture-evaporation process. The co-evaporation is a mixed deposition method in which two or more materials are placed in a respective individual crucible source and a current is applied to both cells simultaneously to evaporate the materials. The mixture-evaporation is a mixed deposition method in which two or more materials are mixed in one crucible source before evaporating them, and a current is applied to the cell to evaporate the materials. Further, if the first and the second host compounds are present in the same layer or different layers in an organic electroluminescent device, the two host compounds may individually form films. For example, the second host compound may be deposited after depositing the first host compound.

The present disclosure may provide a display system comprising the plurality of host materials comprising the compound represented by formula 1 or 1′ and the compound represented by formula 2 or 2′. In other words, it is possible to manufacture a display system or a lighting system by using the plurality of host materials of the present disclosure. Specifically, it is possible to produce a display system, e.g., a display system for white organic light-emitting devices, smart phones, tablets, notebooks, PCs, TVs. or cars; or a lighting system, e.g., an outdoor or indoor lighting system, by using the plurality of host materials of the present disclosure.

Hereinafter, the preparation method of the compounds according to the present disclosure, the properties thereof, and the properties of the OLED comprising the plurality of host materials of the present disclosure will be explained in detail with reference to the representative compounds of the present disclosure. The following examples only describe the properties of the OLED comprising the compound or a plurality of host materials according to the present disclosure, but the present disclosure is not limited to the following examples.

### Example 1: Preparation of Compound H1-235-D14

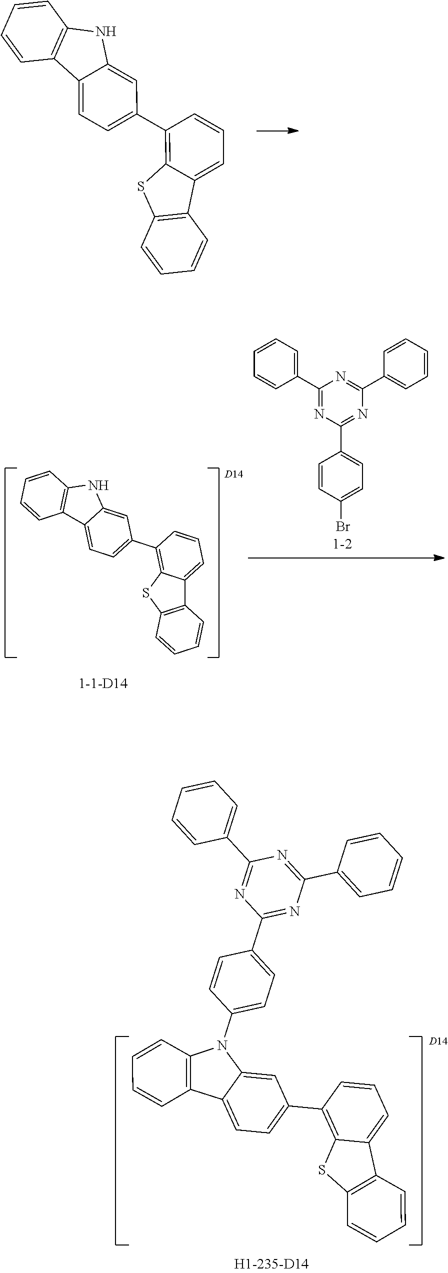

### Synthesis of Compound 1-1-D14

In a flask, 2-(dibenzo[b,d]thiophen-4-yl)-9H-carbazole (15.0 g, 42.9 mmol) and benzene-D6 (1.0 kg, 11.88 mol) were added, and the mixture was stirred under reflux. Triflic acid (50.7 g, 337.8 mmol) was added to the mixture at 70° C. After 4 hours, the mixture was cooled to room temperature. 30 mL of D₂O was added thereto, and the mixture was stirred for 10 minutes. The mixture was neutralized with an aqueous K₃PO₄ solution, and an organic layer was extracted with ethyl acetate. After removing the residual moisture with magnesium sulfate, the residue was distilled under reduced pressure, and separated by column chromatography to obtain compound 1-1-D14 (12 g, yield: 77.0%).

### Synthesis of Compound H1-235-D14

In a flask, compound 1-1-D14 (4 g, 11.05 mmol), compound 1-2 (5.15 g, 13.26 mmol), Pd(OAc)₂ (0.12 g, 0.55 mmol), 2-dicyclohexylphosphino-2′,6′-dimethoxybiphenyl (S-phos) (0.45 g, 1.105 mmol), NaOt-bu (2.65 g, 27.62 mmol), and 150 mL of o-xylene were added, and the mixture was heated at 180° C. for 4 hours. Thereafter, the mixture was cooled to room temperature, and methanol was added thereto. The resulting solid was filtered under reduced pressure. The obtained solid was separated by column chromatography to obtain compound H1-235-D14 (4.9 g, yield: 66.2%).

| Compound | MW | M.P. |
|---|---|---|
| H1-235-D14 | 670 | 282° C. |

### Example 2: Preparation of Compound H1-232-D14

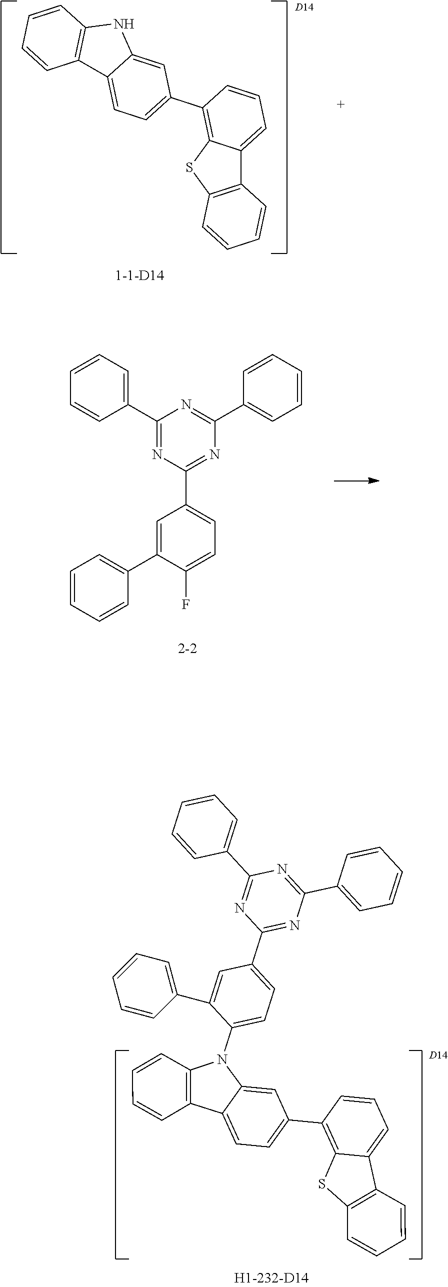

In a flask, compound 1-1-D14 (8.9 g, 24.55 mmol), compound 2-2 (10.4 g, 25.77 mmol), 4-dimethylaminopyridine (DMAP) (1.5 g, 12.27 mmol), cesium fluoride (CsF) (9.32 g, 61.35 mmol), and 300 mL of N-methyl-2-pyrrolidone (NMP) were added, and the mixture was heated at 200° C. After 2 hours, the mixture was cooled to room temperature, and 1 L of methanol and 400 mL of distilled water were added thereto. The resulting solid was filtered under reduced pressure. The obtained solid was separated by column chromatography to obtain compound H1-232-D14 (10 g, yield: 54.6%).

| Compound | MW | M.P. |
|---|---|---|
| H1-232-D14 | 746.2 | 300° C. |

### Example 3: Preparation of Compound H1-211-D12

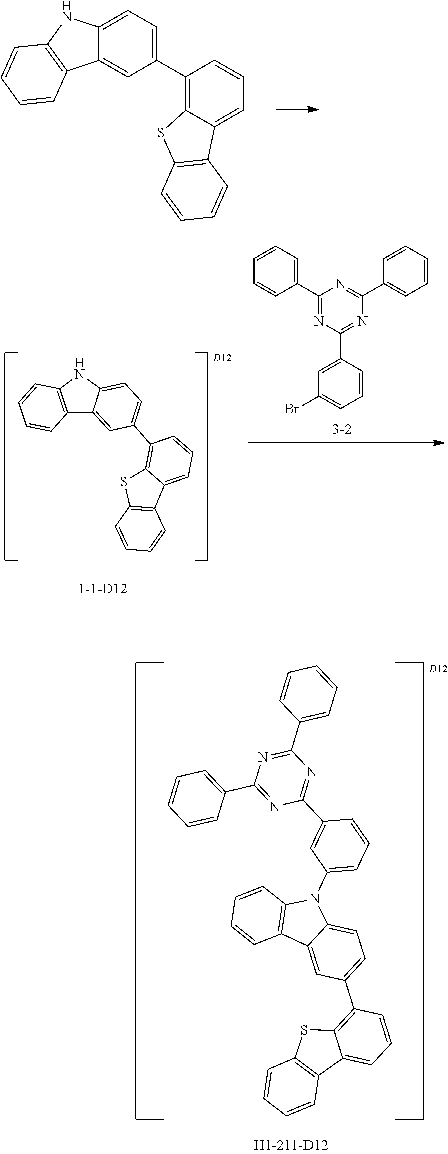

### Synthesis of Compound 1-1-D12

In a flask, 2-(dibenzo[b,d]thiophen-4-yl)-9H-carbazole (15.0 g, 42.9 mmol) and benzene-D6 (1.2 kg, 14.26 mol) were added, and the mixture was stirred under reflux. Triflic acid (50.7 g, 337.8 mmol) was added to the mixture at 70° C. After 4 hours, the mixture was cooled to room temperature. 30 mL of D₂O was added thereto, and the mixture was stirred for 10 minutes. The mixture was neutralized with an aqueous K₃PO₄ solution, and an organic layer was extracted with ethyl acetate. After removing the residual moisture with magnesium sulfate, the residue was distilled under reduced pressure, and separated by column chromatography to obtain compound 1-1-D12 (11 g, yield: 71.1%).

### Synthesis of Compound H1-211-D12

In a flask, compound 1-1-D12 (4 g, 11.05 mmol), compound 3-2 (5.15 g, 13.26 mmol), Pd(OAc)₂ (0.12 g, 0.55 mmol), S-phos (0.45 g, 1.105 mmol), NaOt-bu (2.65 g, 27.62 mmol), and 150 mL of o-xylene were added, and the mixture was heated to 185° C. for 4 hours. Thereafter, the mixture was cooled to room temperature, and methanol was added thereto. The resulting solid was filtered under reduced pressure. The obtained solid was separated by column chromatography to obtain compound H1-211-D12 (4.8 g, yield: 64.7%).

| Compound | MW | M.P. |
|---|---|---|
| H1-211-D12 | 668 | 242° C. |

### Example 4: Preparation of Compound H1-232

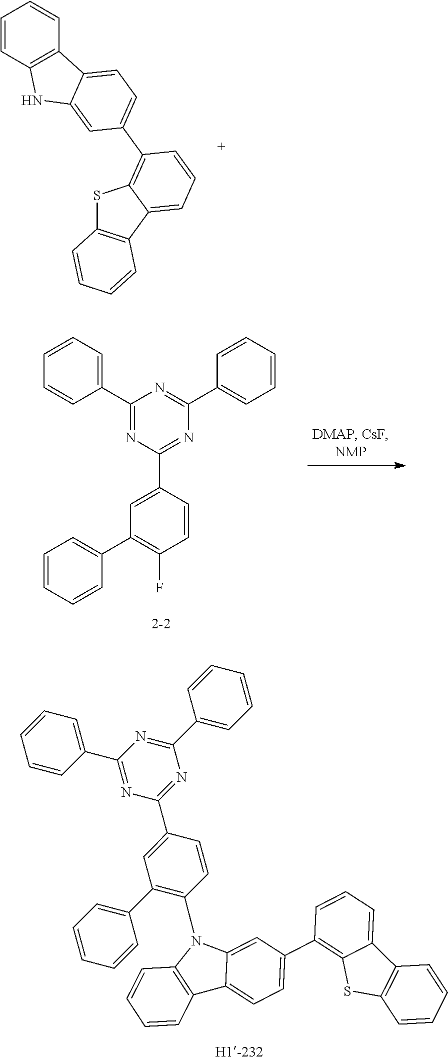 [SMILES: c1ccc2c(c1)[nH]c1cc(-c3cccc4c3sc3ccccc34)ccc12 ; Fc1ccc(-c2nc(-c3ccccc3)nc(-c3ccccc3)n2)cc1-c1ccccc1 ; c1ccc(-c2nc(-c3ccccc3)nc(-c3ccc(-n4c5ccccc5c5ccc(-c6cccc7c6sc6ccccc67)cc54)c(-c4ccccc4)c3)n2)cc1]

In a flask, 2-(dibenzo[b,d]thiophen-4-yl)-9H-carbazole (8.4 g, 24.0 mmol), compound 2-2 (10.8 g, 26.8 mmol), DMAP (1.5 g, 12.0 mmol), and CsF (9.1 g, 59.9 mmol) were dissolved in 250 mL of NMP, and the mixture was stirred under reflux for 2 hours. After completion of the reaction, the mixture was crystallized with H₂O, and separated by column chromatography to obtain compound H1′-232 (15.0 g, yield: 86%).

| Compound | MW | M.P. |
|---|---|---|
| H1′-232 | 732.91 | 298.5° C. |

### Example 5: Preparation of Compound H2-83-D25

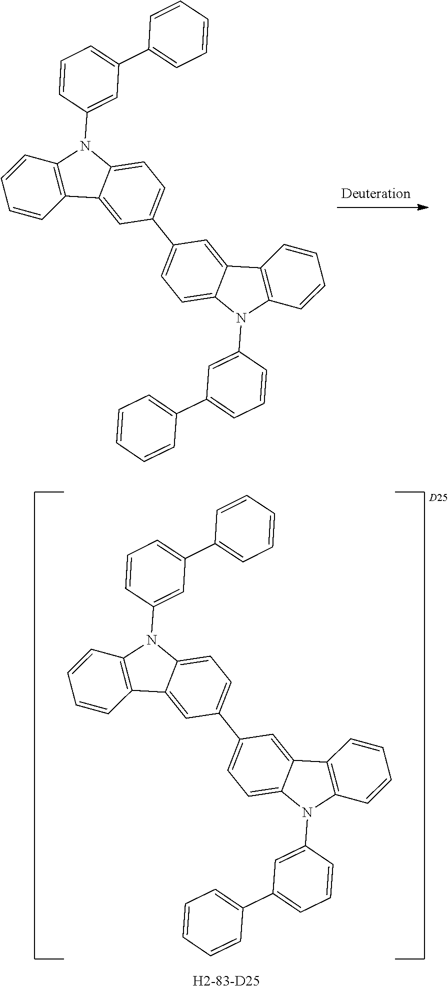

In a flask, 9,9′-di([1,1′-biphenyl]-3-yl)-9H,9′H-3,3′-bicarbazole (15.0 g, 42.9 mmol) and 900 mL of benzene-D6 were added, and the mixture was heated. Thereafter, triflic acid (25.4 g, 169.5 mmol) was added to the mixture at 60° C. After 3 hours, the mixture was cooled to room temperature. 30 mL of D₂O was added thereto, and the mixture was stirred for 10 minutes. The mixture was neutralized with an aqueous K₃PO₄ solution, and an organic layer was extracted with ethyl acetate. After removing the residual moisture with magnesium sulfate, the residue was distilled under reduced pressure, and then separated by column chromatography to obtain compound H2-83-D25 (12 g, yield: 77.0%).

| Compound | MW | M.P. |
|---|---|---|
| H2-83-D25 | 661 | 152° C. |

### Example 6: Preparation of Compound H2-45-D21

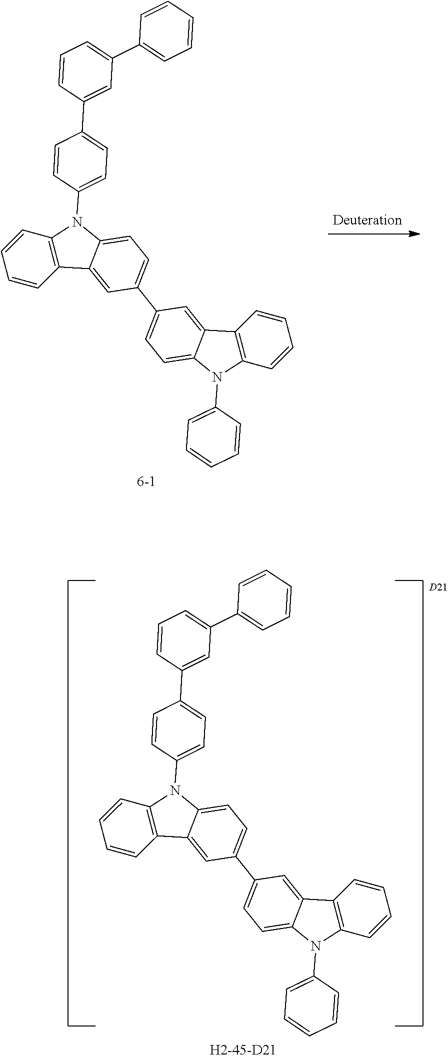

In a flask, compound 6-1 (0.5 g, 0.78 mmol) and 4 mL of benzene-D6 were added, and the mixture was heated. Thereafter, triflic acid (0.42 g, 2.83 mmol) was added to the mixture at 60° C. After 17 hours, the mixture was cooled to room temperature. 0.5 mL of D₂O was added to the mixture, and the mixture was stirred for 10 minutes. The mixture was neutralized with an aqueous K₃PO₄ solution, and an organic layer was extracted with ethyl acetate. After removing the residual moisture with magnesium sulfate, the residue was distilled under reduced pressure, and separated by column chromatography to obtain compound H2-45-D21 (0.3 g, yield: 58.1%).

| Compound | MW | M.P. |
|---|---|---|
| H2-45-D21 | 661 | 152° C. |

### Device Examples 1 and 2: Producing a Green OLED Deposited with a Plurality of Host Materials According to the Present Disclosure

OLEDs according to the present disclosure were produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI-1 was introduced into a cell of the vacuum vapor deposition apparatus, and compound HT-1 was introduced into another cell of the vacuum vapor deposition apparatus. The two materials were evaporated at different rates, and compound HI-1 was deposited in a doping amount of 3 wt % based on the total amount of compound HI-1 and compound HT-1 to form a hole injection layer having a thickness of 10 nm on the ITO substrate. Next, compound HT-1 was deposited on the hole injection layer to form a first hole transport layer having a thickness of 80 nm. Compound HT-2 was then introduced into another cell of the vacuum vapor deposition apparatus and was evaporated by applying an electric current to the cell, thereby forming a second hole transport layer having a thickness of 30 nm on the first hole transport layer. After forming the hole injection layer and the hole transport layers, a light-emitting layer was formed thereon as follows: The first host compound and the second host compound shown in Table 1 below were introduced into two cells of the vacuum vapor deposition apparatus as hosts, and compound D-130 was introduced into another cell as a dopant. The two host materials were evaporated at a rate of 2:1 (the first host:the second host) and the dopant material was simultaneously evaporated at a different rate, and the dopant was deposited in a doping amount of 10 wt % based on the total amount of the hosts and the dopant to form a light-emitting layer having a thickness of 40 nm on the second hole transport layer. Thereafter, compound ETL-1 and compound EIL-1 were evaporated in a weight ratio of 40:60 in each of two other cells to deposit an electron transport layer having a thickness of 35 nm on the light-emitting layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr. The compounds used in Device Examples 1 and 2 are as follows.

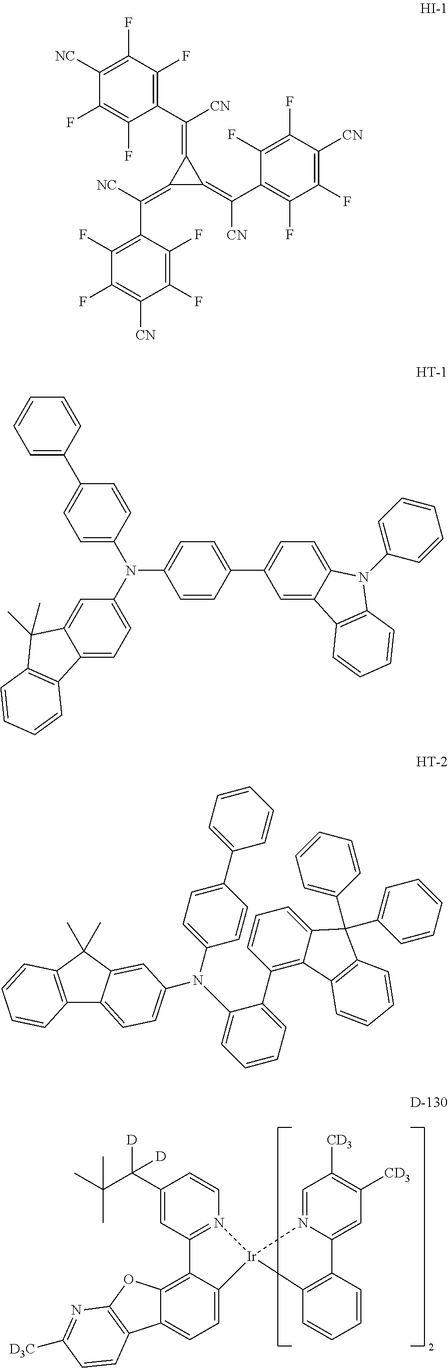

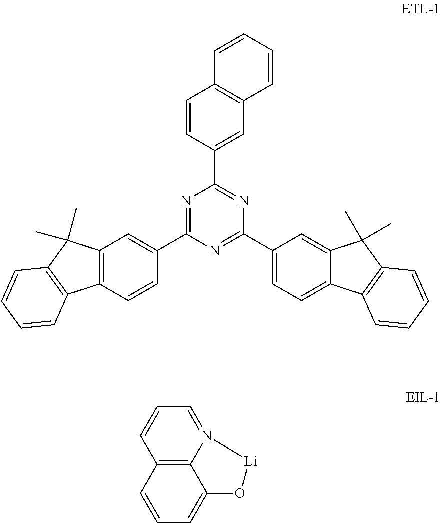

### Comparative Examples 1 and 2: Producing an OLED Comprising Comparative Compounds as Hosts

OLEDs were produced in the same manner as in Device Examples 1 and 2, except that the host compounds shown in Table below were used as the hosts of the light-emitting layer.

The driving voltage, luminous efficiency, and light-emitting color at a luminance of 1000 nit, and the time taken for luminance to decrease from 100% to 95% (lifetime; T95) at a luminance of 20,000 nit of the OLEDs produced in Device Examples 1 and 2, and Comparative Examples 1 and 2 are provided in Table 1 below.

TABLE 1

| | | | Dri- | Lumi- | | |
|---|---|---|---|---|---|---|
| | | | ving | nous | Light- | |
| | | | Vol- | Effi- | Emit- | Lifetime |
| | First | Second | tage | ciency | ting | (T95) |
| | Host | Host | [V] | [cd/A] | Color | [hr] |
| Device | H1-232- | H2-6 | 3.2 | 106.4 | Green | 195.8 |
| Example 1 | D14 | | | | | |
| Device | H1-235- | H2-6 | 3.2 | 105.8 | Green | 226.6 |
| Example 2 | D14 | | | | | |
| Comparative | H1′-265 | H2-6 | 3.2 | 106.7 | Green | 163.8 |
| Example 1 | | | | | | |
| Comparative | C-1 | H2-6 | 3.2 | 103.2 | Green | 177.5 |
| Example 2 | | | | | | |

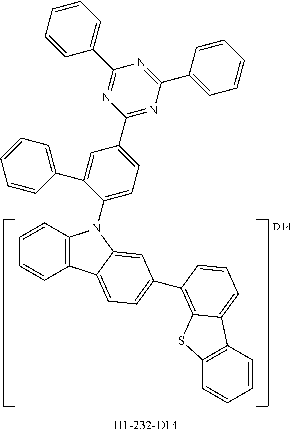

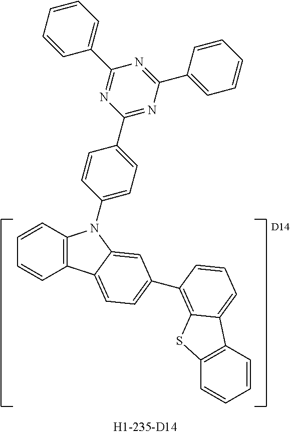

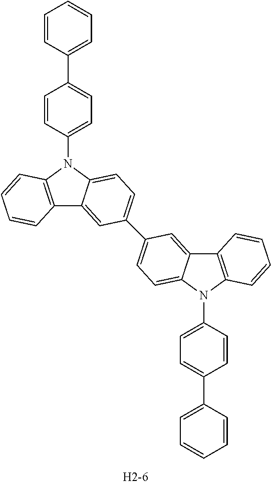 [SMILES: c1ccc(-c2ccc(-n3c4ccccc4c4cc(-c5ccc6c(c5)c5ccccc5n6-c5ccc(-c6ccccc6)cc5)ccc43)cc2)cc1]

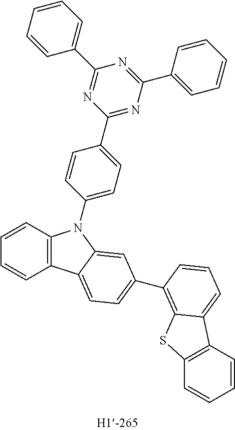 [SMILES: c1ccc(-c2nc(-c3ccccc3)nc(-c3ccc(-n4c5ccccc5c5ccc(-c6cccc7c6sc6ccccc67)cc54)cc3)n2)cc1]

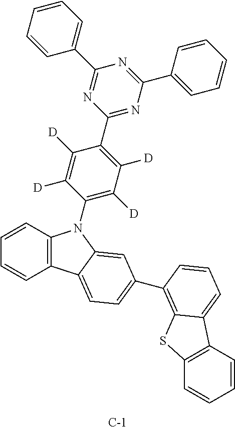

### Device Examples 3 and 4: Producing a Green OLED Deposited with a Plurality of Host Materials According to the Present Disclosure

OLEDs were produced in the same manner as in Device Examples 1 and 2, except that compound HT-3 was used instead of compound HT-2 as the second hole transport layer, and the first and second host compounds shown in Table 2 below were used as the hosts of the light-emitting layer.

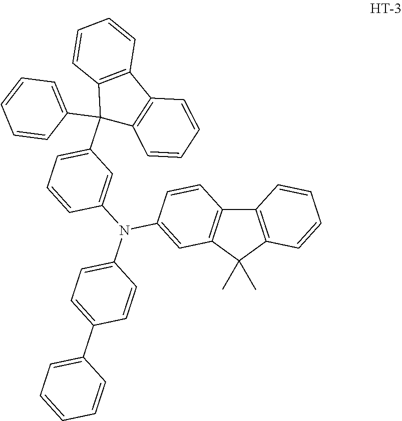 [SMILES: CC1(C)c2ccccc2-c2ccc(N(c3ccc(-c4ccccc4)cc3)c3cccc(C4(c5ccccc5)c5ccccc5-c5ccccc54)c3)cc21]

### Comparative Example 3: Producing an OLED Comprising Comparative Compounds as Hosts

An OLED was produced in the same manner as in Device Example 3, except that the host compounds shown in Table 2 below were used as the hosts of the light-emitting layer.

The driving voltage, luminous efficiency, and light-emitting color at a luminance of 1,000 nit, and the time taken for luminance to decrease from 100% to 95% (lifetime; T95) at a luminance of 20,000 nit of the OLEDs produced in Device Examples 3 and 4, and Comparative Example 3 are provided in Table 2 below.

TABLE 2

| | | | Dri- | Lumi- | | |
|---|---|---|---|---|---|---|
| | | | ving | nous | Light- | |
| | | | Vol- | Effi- | Emit- | Lifetime |
| | First | Second | tage | ciency | ting | (T95) |
| | Host | Host | [V] | [cd/A] | Color | [hr] |
| Device | H1-235- | H2-33 | 3.1 | 105.5 | Green | 260 |
| Example 3 | D14 | | | | | |
| Device | H1-235- | H2-83- | 3.1 | 106.7 | Green | 401 |
| Example 4 | D14 | D25 | | | | |
| Comparative | H1′-265 | H2-33 | 3.1 | 105.7 | Green | 232 |
| Example 3 | | | | | | |

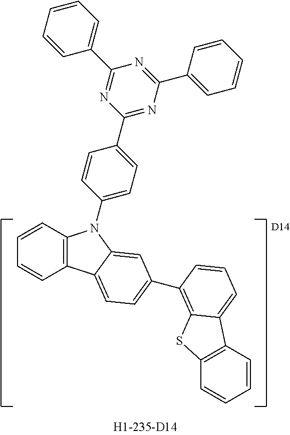

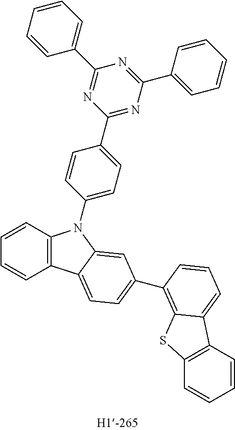 [SMILES: c1ccc(-c2nc(-c3ccccc3)nc(-c3ccc(-n4c5ccccc5c5ccc(-c6cccc7c6sc6ccccc67)cc54)cc3)n2)cc1]

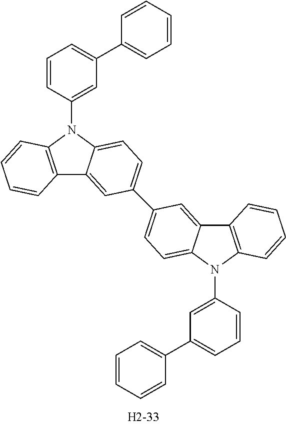 [SMILES: c1ccc(-c2cccc(-n3c4ccccc4c4cc(-c5ccc6c(c5)c5ccccc5n6-c5cccc(-c6ccccc6)c5)ccc43)c2)cc1]

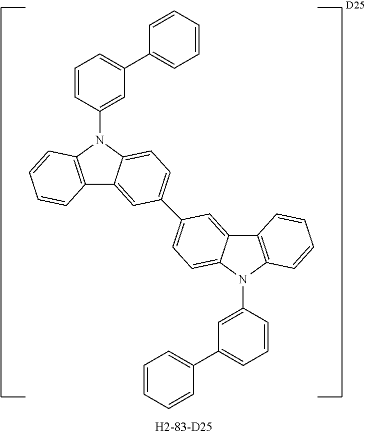

### Device Examples 5 and 6: Producing a Green OLED Deposited with a Plurality of Host Materials According to the Present Disclosure

OLEDs were produced in the same manner as in Device Example 3, except that the first and second host compounds shown in Table 3 below were used as the hosts of the light-emitting layer, and two host materials were evaporated at different rates of 1:2 (the first host:the second host).

### Comparative Example 4: Producing an OLED Comprising Comparative Compounds as Hosts

An OLED was produced in the same manner as in Device Example 5, except that compound 12-33 was used as the second host of the light-emitting layer.

The driving voltage, luminous efficiency, and light-emitting color at a luminance of 1000 nit, and the time taken for luminance to decrease from 100% to 95% (lifetime; T95) at a luminance of 20,000 nit of the OLEDs produced in Device Examples 5 and 6 and Comparative Example 4 are provided in Table 3 below.

TABLE 3

| | | | Dri- | Lumi- | | |
|---|---|---|---|---|---|---|
| | | | ving | nous | | |
| | | | Vol- | Effi- | Light- | Lifetime |
| | First | Second | tage | ciency | Emitting | (T95) |
| | Host | Host | [V] | [cd/A] | Color | [hr] |
| Device | H1′-265 | H2-83 | 3.2 | 106.7 | Green | 365 |
| Example 5 | | | | | | |
| Device | H1′-232 | H2-83- | 3.2 | 106.1 | Green | 355 |
| Example 6 | | D25 | | | | |
| Comparative | H1′-265 | H2-33 | 3.1 | 105.7 | Green | 232 |
| Example 4 | | | | | | |

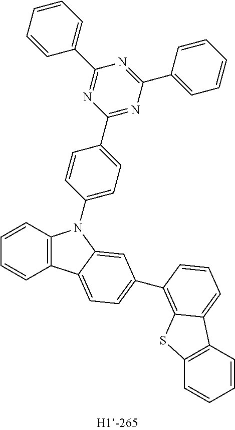 [SMILES: c1ccc(-c2nc(-c3ccccc3)nc(-c3ccc(-n4c5ccccc5c5ccc(-c6cccc7c6sc6ccccc67)cc54)cc3)n2)cc1]

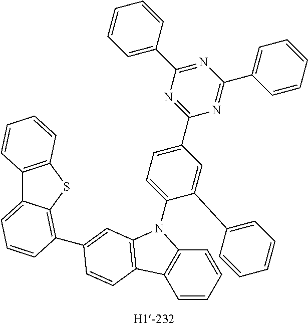 [SMILES: c1ccc(-c2nc(-c3ccccc3)nc(-c3ccc(-n4c5ccccc5c5ccc(-c6cccc7c6sc6ccccc67)cc54)c(-c4ccccc4)c3)n2)cc1]

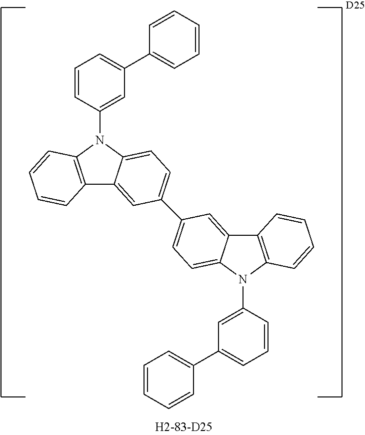

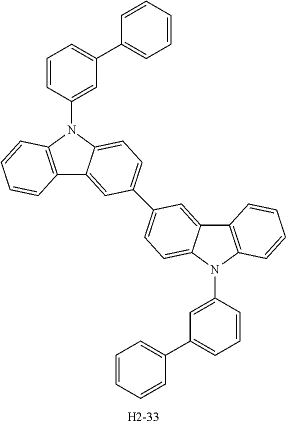 [SMILES: c1ccc(-c2cccc(-n3c4ccccc4c4cc(-c5ccc6c(c5)c5ccccc5n6-c5cccc(-c6ccccc6)c5)ccc43)c2)cc1]

It was confirmed that the OLED using the plurality of host materials according to the present disclosure exhibits excellent lifetime properties while exhibiting the similar level of luminous properties as compared to the OLED comprising the combination of the conventional hosts.

### Device Example 7: Producing a Blue OLED Comprising an Organic Electroluminescent Compound According to the Present Disclosure in an Electron Buffer Layer

A blue OLED according to the present disclosure was produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI-1 was introduced into a cell of the vacuum vapor deposition apparatus, and compound HT-1 was introduced into another cell of the vacuum vapor deposition apparatus. The two materials were evaporated at different rates, and compound HI-1 was deposited in a doping amount of 3 wt % based on the total amount of compound HI-1 and compound HT-1 to form a hole injection layer having a thickness of 10 nm on the ITO substrate. Next, compound HT-1 was deposited to form a first hole transport layer having a thickness of 75 nm on the hole injection layer. Compound HT-4 was then introduced into another cell of the vacuum vapor deposition apparatus and was evaporated by applying an electric current to the cell, thereby forming a second hole transport layer having a thickness of 5 nm on the first hole transport layer. After forming the hole injection layer and the hole transport layers, a light-emitting layer was formed thereon as follows: Compound H-a was introduced into a cell of the vacuum vapor deposition apparatus as a host, and compound D-a was introduced into another cell as a dopant. The host material and the dopant material were evaporated at different rates, and the dopant was deposited in a doping amount of 2 wt % based on the total amount of the host and the dopant to form a light-emitting layer having a thickness of 20 nm on the second hole transport layer. Compound H1-235-D14 was then introduced into a cell of the vacuum vapor deposition apparatus and was evaporated to form an electron buffer layer having a thickness of 5 nm on the light-emitting layer. Compound ETL-1 and compound EIL-1 were evaporated in a weight ratio of 4:6 in each of two other cells to deposit an electron transport layer having a thickness of 30 nm on the electron buffer layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr.

The minimum time taken for luminance to decrease from 100% to 95% at a luminance of 1,770 nit (lifetime; T95) of the produced OLED was 56.5 hours.

### Comparative Example 5: Producing a Blue OLED Comprising a Comparative Compound in the Electron Buffer Layer

An OLED was produced in the same manner as in Device Example 7, except that compound H1′-265 was used as the material of the electron buffer layer.

The minimum time taken for luminance to decrease from 100% to 95% at a luminance of 1,770 nit (lifetime; T95) of the produced OLED was 47.1 hours.

The compounds used in Device Example 7 and Comparative Example 5 are as follows.

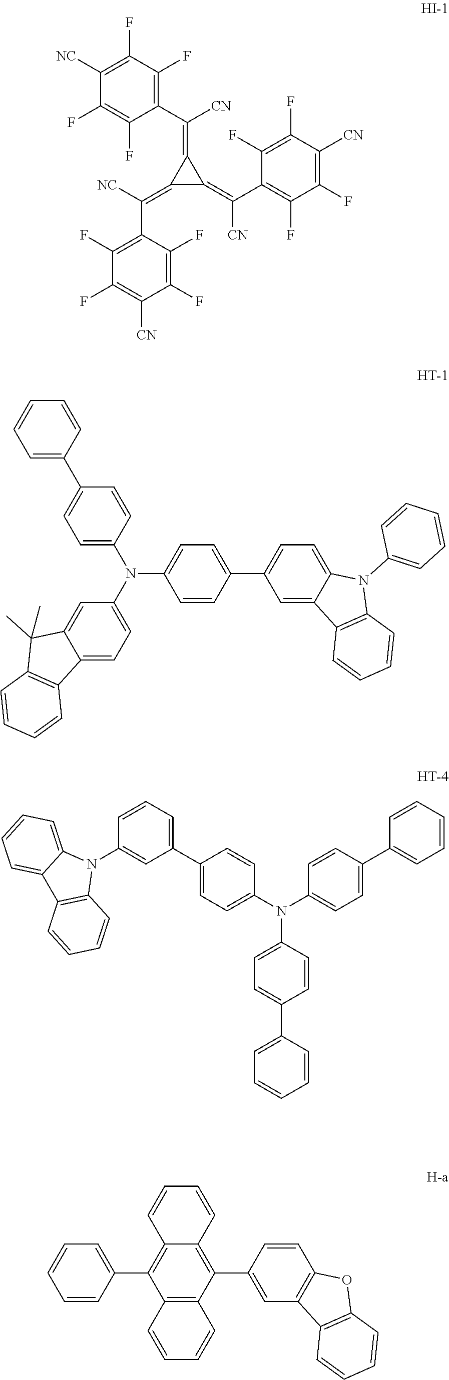 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccccc4)cc3)c3ccc(-c4cccc(-n5c6ccccc6c6ccccc65)c4)cc3)cc2)cc1 ; c1ccc(-c2c3ccccc3c(-c3ccc4oc5ccccc5c4c3)c3ccccc23)cc1]

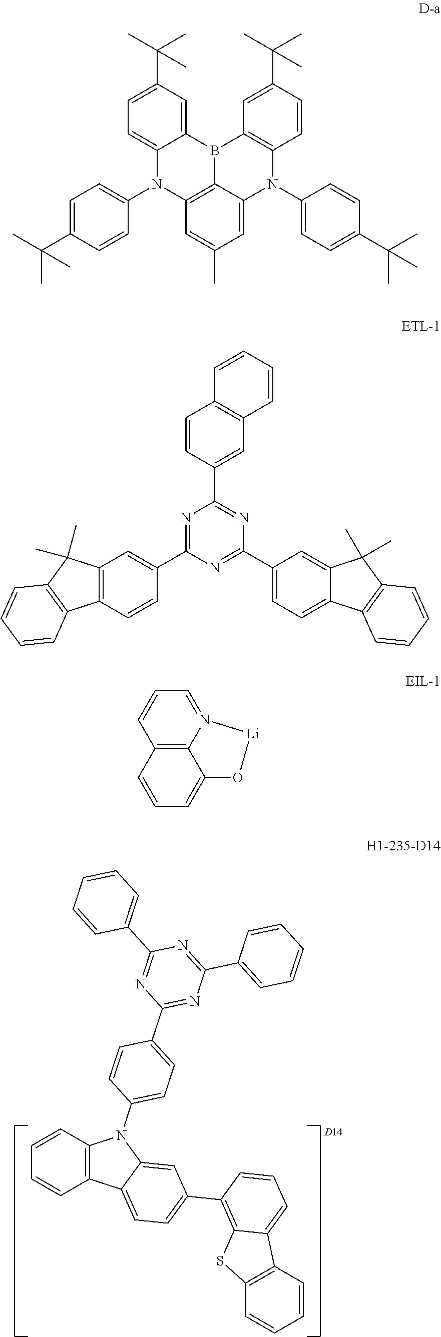

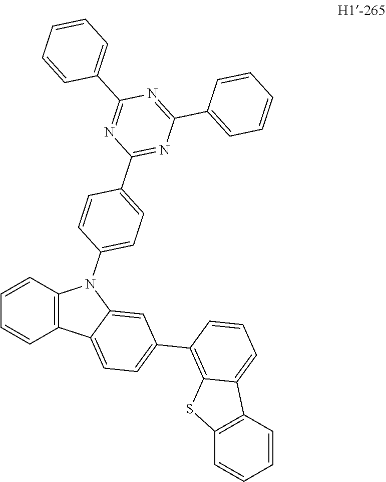 [SMILES: c1ccc(-c2nc(-c3ccccc3)nc(-c3ccc(-n4c5ccccc5c5ccc(-c6cccc7c6sc6ccccc67)cc54)cc3)n2)cc1]

From Device Example 7 and Comparative Example 5, it can be confirmed that the OLED comprising the organic electroluminescent compound according to the present disclosure in an electron buffer layer has more improved lifetime property compared to the case where the conventional compound was used.

### Device Example 8: Producing a Green OLED Comprising an Organic Electroluminescent Compound According to the Present Disclosure as a Host of a Light-Emitting Layer

An OLED according to the present disclosure was produced. A transparent electrode indium tin oxide (ITO) thin film (10 Ω/sg) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI-1 was introduced into a cell of the vacuum vapor deposition apparatus, and compound HT-1 was introduced into another cell of the vacuum vapor deposition apparatus. The two materials were evaporated at different rates, and compound HI-1 was deposited in a doping amount of 3 wt % based on the total amount of compound HI-1 and compound HT-1 to form a hole injection layer having a thickness of 10 nm on the ITO substrate. Next, compound HT-1 was deposited on the hole injection layer to form a first hole transport layer having a thickness of 80 nm. Compound HT-2 was then introduced into another cell of the vacuum vapor deposition apparatus and was evaporated by applying an electric current to the cell, thereby forming a second hole transport layer having a thickness of 30 nm on the first hole transport layer. After forming the hole injection layer and the hole transport layers, a light-emitting layer was formed thereon as follows: Compound H2-83-D25 was introduced into a cell of the vacuum vapor deposition apparatus as a host, and compound D-50 was introduced into another cell as a dopant. The dopant was deposited in a doping amount of 10 wt % based on the total amount of the host and the dopant to form a light-emitting layer having a thickness of 30 nm on the second hole transport layer. Thereafter, compound HBL-1 was introduced into a cell of the vacuum vapor deposition apparatus and was evaporated to deposit a hole blocking layer having a thickness of 10 nm on the light-emitting layer. Compound ETL-1 and compound EIL-1 were evaporated in a weight ratio of 4:6 in each of two other cells to deposit an electron transport layer having a thickness of 35 nm on the hole blocking layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr. The compounds used in Device Example 8 are as follows.

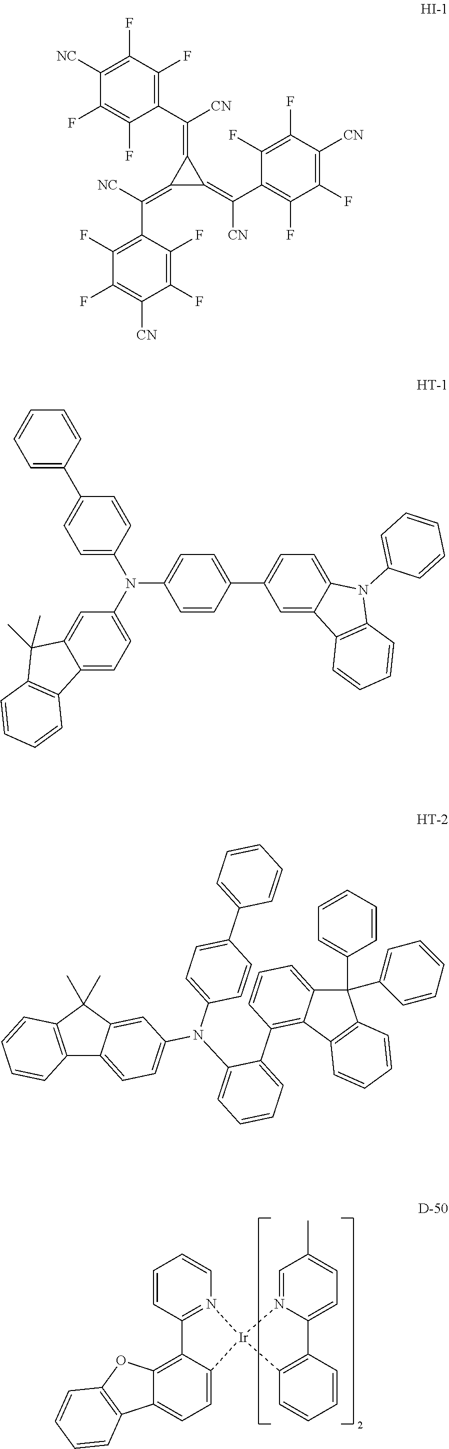

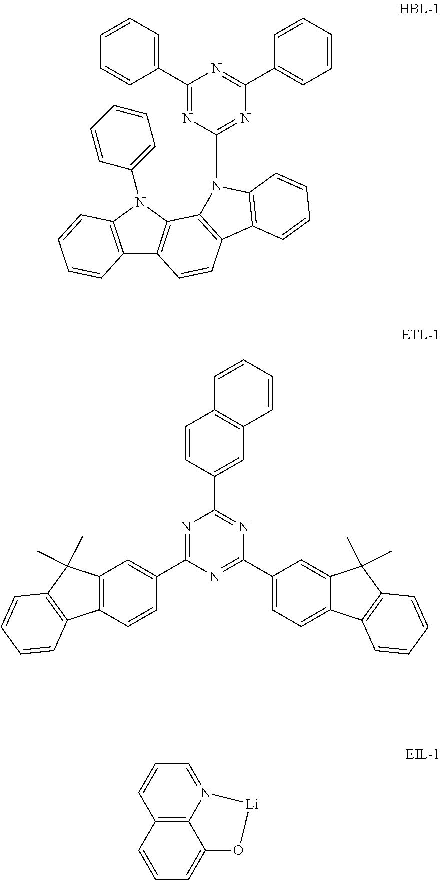

### Comparative Examples 6 and 7: Producing an OLED Comprising a Comparative Compound as the Host of the Light-Emitting Layer

OLEDs were produced in the same manner as in Device Example 8, except that the compound shown in Table 4 below was used as the host of the light-emitting layer.

The driving voltage, luminous efficiency, and light-emitting color at a luminance of 1,000 nit, and the time taken for luminance to decrease from 100% to 95% (lifetime; T95) at a luminance of 20,000 nit of the OLEDs produced in Device Example 8, and Comparative Examples 6 and 7 are provided in Table 4 below.

TABLE 4

| | | Driving | Luminous | Light- | Lifetime |
|---|---|---|---|---|---|
| | | Voltage | Efficiency | Emitting | (T95) |
| | Host | [V] | [cd/A] | Color | [hr] |
| Device | H2-83- | 3.5 | 82.2 | Green | 12.7 |
| Example 8 | D25 | | | | |
| Comparative | H2-33 | 3.4 | 82.5 | Green | 10.4 |
| Example 6 | | | | | |
| Comparative | C-2 | 3.6 | 79.6 | Green | 9.0 |
| Example 7 | | | | | |

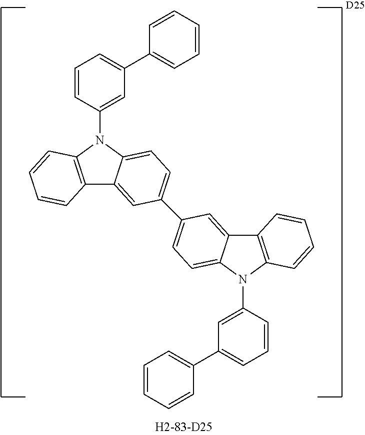

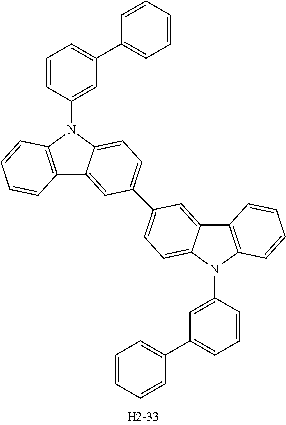 [SMILES: c1ccc(-c2cccc(-n3c4ccccc4c4cc(-c5ccc6c(c5)c5ccccc5n6-c5cccc(-c6ccccc6)c5)ccc43)c2)cc1]

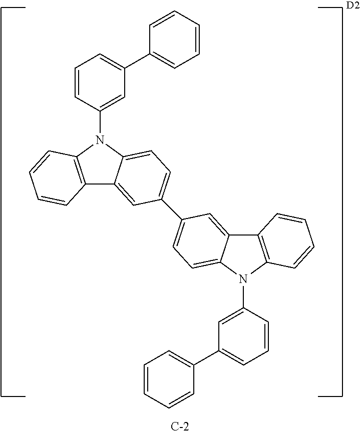

It was confirmed that the OLED using the organic electroluminescent compound according to the present disclosure exhibits excellent lifetime properties while exhibiting the similar level of luminous properties as compared to the OLED comprising the conventional compound.

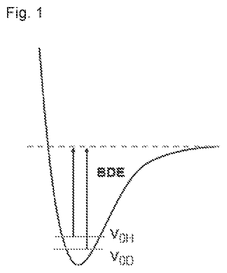

The lifetime of the green OLED is generally shorter than that of the red OLED. In order to improve the lifetime properties of a green OLED, a compound having a deuterated moiety was used in the present disclosure. Without wishing to be bound by theory, when the organic electroluminescent compound is substituted with deuterium, the bond dissociation energy (BDE) in the compound is increased by lowering the zero point vibration energy of the compound, which can improve the stability of the compound. FIG. 1 illustrates a graph showing the increase in bond dissociation energy according to deuteration.

1. A plurality of host materials comprising at least one first host compound and at least one second host compound, wherein the first host compound is represented by the following formula 1, and the second host compound is represented by the following formula 2, at least one of formula 1 and formula 2 contains deuterium, and the first host compound and the second host compound are different from each other:
[A]Dⁿ¹-[B]Dⁿ² (1)
in formula 1,
A represents *-L₁-HAr;
L₁ represents a single bond, a substituted or unsubstituted (C1-C30)alkylene, a substituted or unsubstituted (C6-C30)arylene, or a substituted or unsubstituted (3- to 30-membered)heteroarylene;
HAr represents a substituted or unsubstituted nitrogen-containing (3- to 30-membered)heteroaryl;
B is represented by the following formula 1-a: 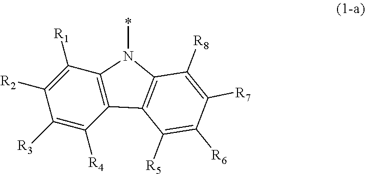
in formula 1-a,
R₁ to R₈, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl, with the proviso that at least one of R₁ to R₈ is represented by the following formula 1-b: 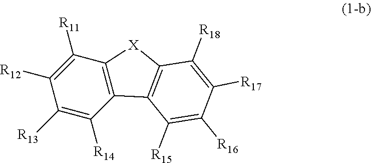
in formula 1-b,
X represents O, S, CR₂₁R₂₂, SiR₂₃R₂₄, or NR₂₅;
R₁₁ to R₁₈, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl, with the proviso that any one of R₁₁ to R₁₈ is linked to formula 1-a;
R₂₁ to R₂₅, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl, or may be linked to an adjacent substituent(s) to form a ring(s);
[A]Dⁿ¹ and [B]Dⁿ² represent that A is substituted with n1 deuterium, and B is substituted with n2 deuterium, respectively; n1 and n2, each independently, represent an integer of 0 to 50, with the proviso that when formula 1 contains deuterium, at least one of n1 and n2 is an integer of 5 or more; and
A and B are linked to each other at the position of *; 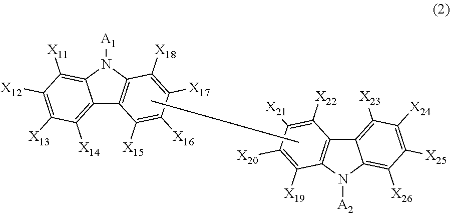
in formula 2,
A₁ and A₂, each independently, represent a substituted or unsubstituted (C6-C30)aryl, a substituted or unsubstituted dibenzofuranyl, a substituted or unsubstituted dibenzothiophenyl, or a substituted or unsubstituted carbazolyl;
X₁₁ to X₂₆, each independently, represent hydrogen, deuterium, a substituted or unsubstituted (C6-C30)aryl, or a substituted or unsubstituted (3- to 30-membered)heteroaryl, or may be linked to an adjacent substituent(s) to form a ring(s);
any one of X₁₁ to X₁₈ is linked to any one of X₁₉ to X₂₆ to form a single bond; and
with the proviso that when formula 2 contains deuterium, at least four of X₁₁ to X₂₆ are deuterium, and at least one of X₁₁, X₁₈, X₁₉, and X₂₆ is deuterium.

2. The plurality of host materials according to claim 1, wherein formula 1 contains deuterium, and the sum of n1 and n2 is an integer of 5 to 50.

3. The plurality of host materials according to claim 1, wherein formula 1 does not contain deuterium, and formula 2 contains deuterium.

4. The plurality of host materials according to claim 1, wherein the substituent(s) of the substituted alkyl(ene), the substituted aryl(ene), the substituted heteroaryl(ene), the substituted nitrogen-containing heteroaryl, the substituted cycloalkyl, the substituted cycloalkenyl, the substituted heterocycloalkyl, the substituted dibenzofuranyl, the substituted dibenzothiophenyl, and the substituted carbazolyl, each independently, are at least one selected from the group consisting of deuterium; a halogen; a cyano; a carboxyl; a nitro; a hydroxyl; a phosphineoxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of deuterium and a (C6-C30)aryl(s); a (C6-C30)aryl unsubstituted or substituted with at least one of deuterium and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldiyl(C6-C30)arylsilyl; a fused ring group of a (C3-C30) aliphatic ring(s) and a (C6-C30) aromatic ring(s); an amino; a mono- or di-(C1-C30)alkylamino; a mono- or di-(C2-C30)alkenylamino; a mono- or di-(C6-C30)arylamino unsubstituted or substituted with a (C1-C30)alkyl(s); a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

5. The plurality of host materials according to claim 1, wherein B is represented by at least one of the following formulas B-1 to B-16: 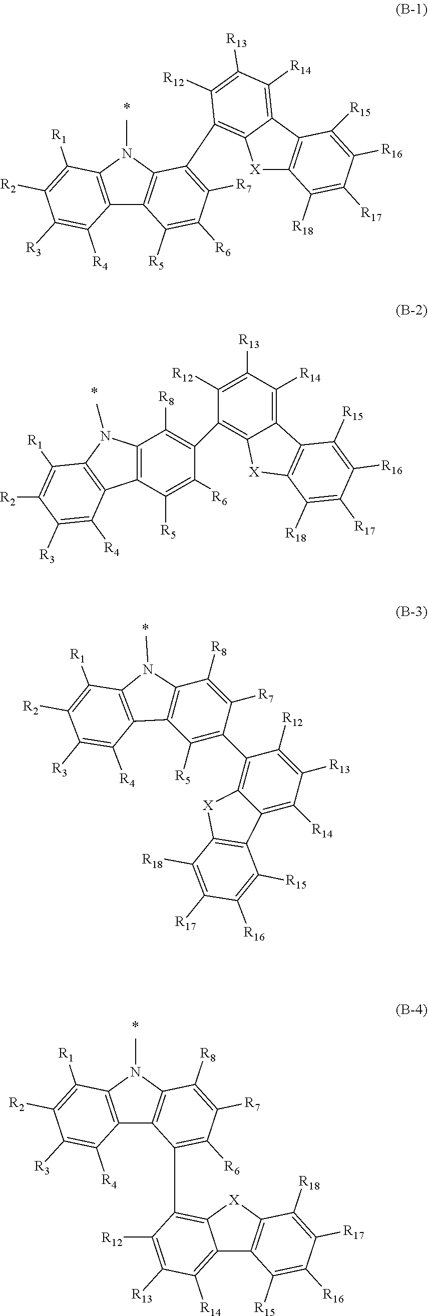 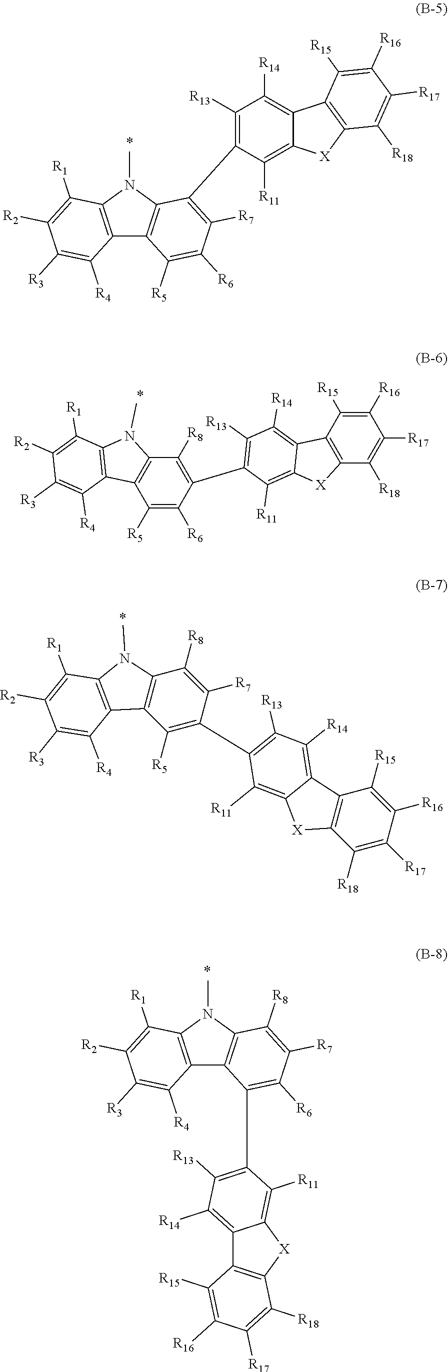 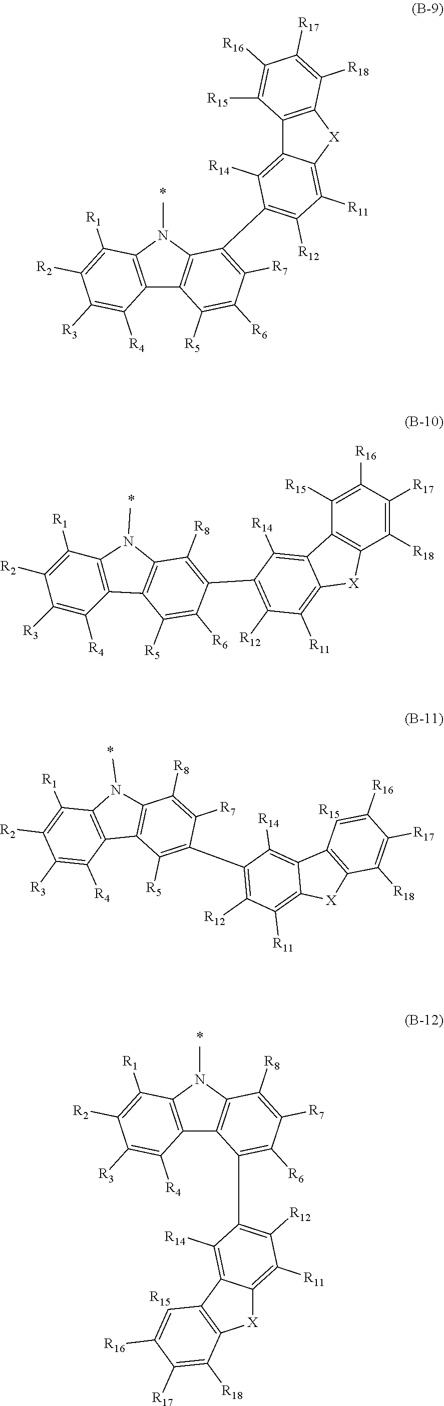 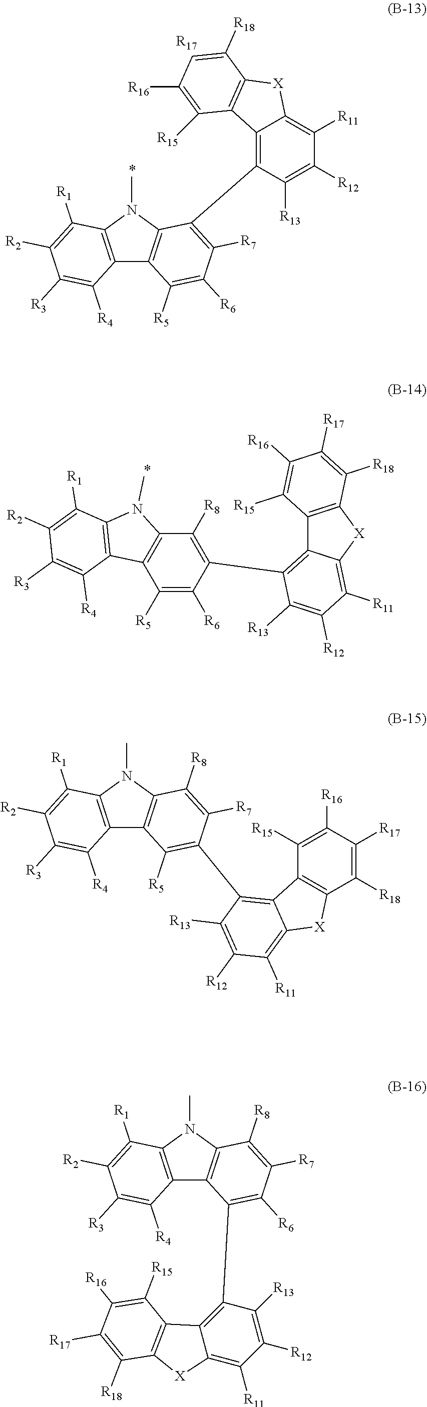
in formulas B-1 to B-16,
R₁ to R₈, R₁₁ to R₁₈, and X are as defined in claim 1.

6. The plurality of host materials according to claim 1, wherein HAr of formula 1 represents a substituted or unsubstituted triazinyl, a substituted or unsubstituted pyridyl, a substituted or unsubstituted pyrimidinyl, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted quinolyl, a substituted or unsubstituted benzoquinolyl, a substituted or unsubstituted isoquinolyl, a substituted or unsubstituted benzoisoquinolyl, a substituted or unsubstituted triazolyl, a substituted or unsubstituted pyrazolyl, a substituted or unsubstituted naphthyridinyl, a substituted or unsubstituted triazanaphthyl, a substituted or unsubstituted benzofuropyrimidinyl, or a substituted or unsubstituted benzothienopyrimidinyl.

7. The plurality of host materials according to claim 1, wherein L₁ of formula 1 represents a single bond, or is represented by any one selected from the group consisting of the followings: 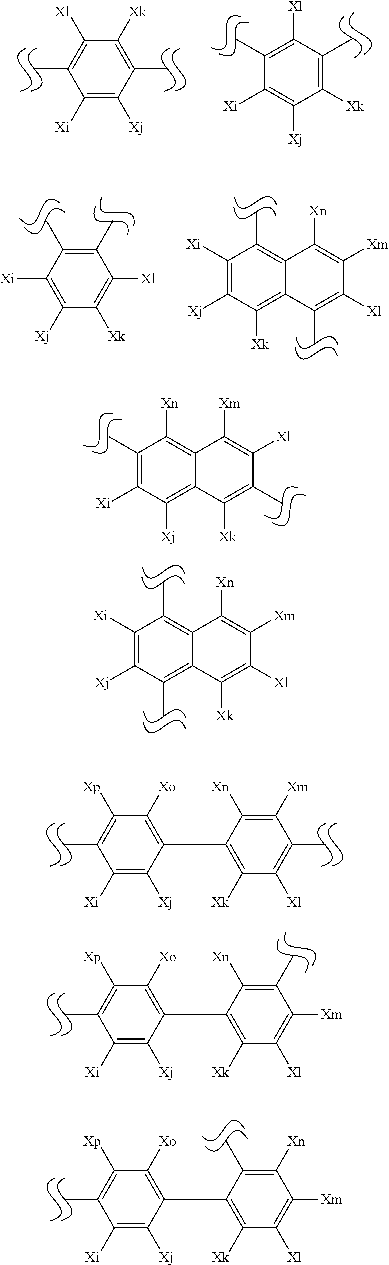 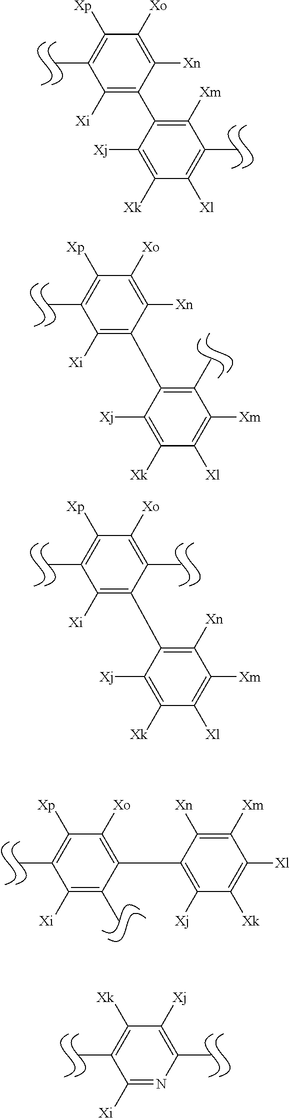
wherein,
Xi to Xp, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C30)aryl, a substituted or unsubstituted (3- to 30-membered)heteroaryl, —NR₂₆R₂₇, or —SiR₂₈R₂₉R₃₀; or may be linked to an adjacent substituent(s) to form a ring(s); and
R₂₆ to R₃₀, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl, or may be linked to an adjacent substituent(s) to form a ring(s).

8. The plurality of host materials according to claim 1, wherein formula 2 is represented by at least one of the following formulas 2-1 to 2-8: 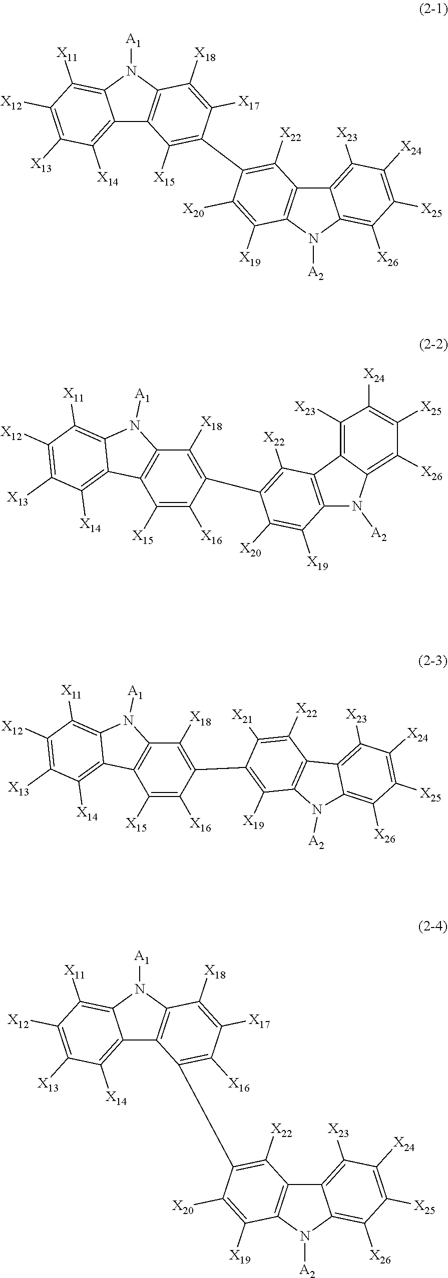 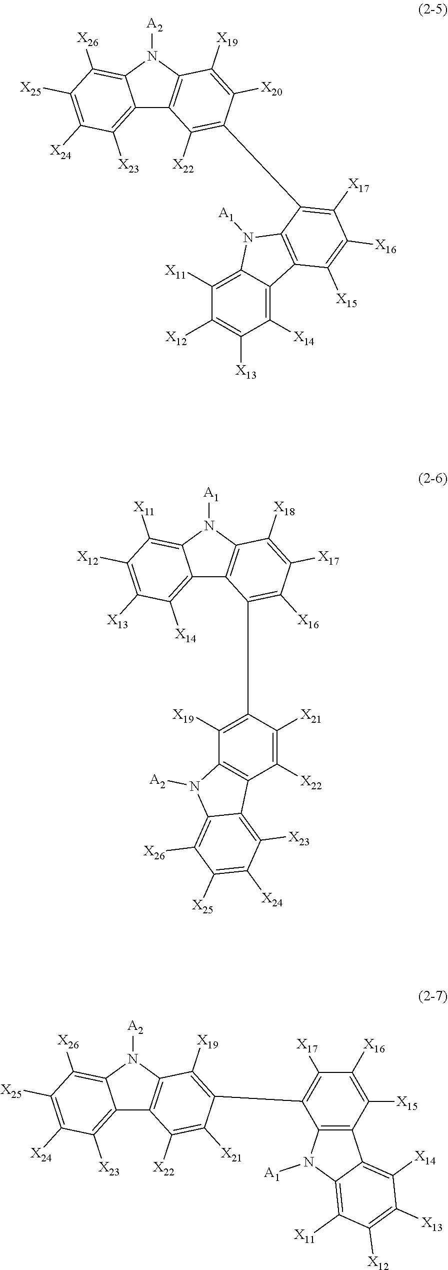 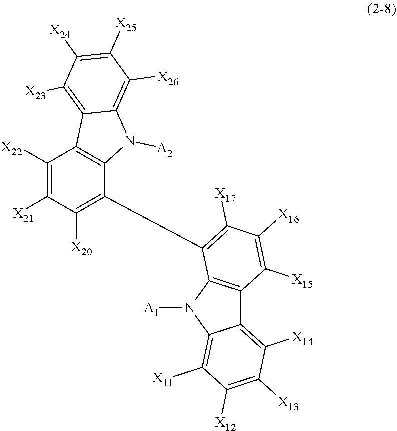
wherein,
A₁, A₂, and X₁₁ to X₂₆ are as defined in claim 1.

9. The plurality of host materials according to claim 1, wherein A₁ and A₂ of formula 2, each independently, represent a substituted or unsubstituted phenyl, a substituted or unsubstituted biphenyl, a substituted or unsubstituted terphenyl, a substituted or unsubstituted naphthyl, a substituted or unsubstituted fluorenyl, a substituted or unsubstituted benzofluorenyl, a substituted or unsubstituted triphenylenyl, a substituted or unsubstituted fluoranthenyl, a substituted or unsubstituted phenanthrenyl, a substituted or unsubstituted dibenzofuranyl, a substituted or unsubstituted carbazolyl, or a substituted or unsubstituted dibenzothiophenyl.

10. The plurality of host materials according to claim 1, wherein the compound represented by formula 1 is at least one selected from the following compounds: 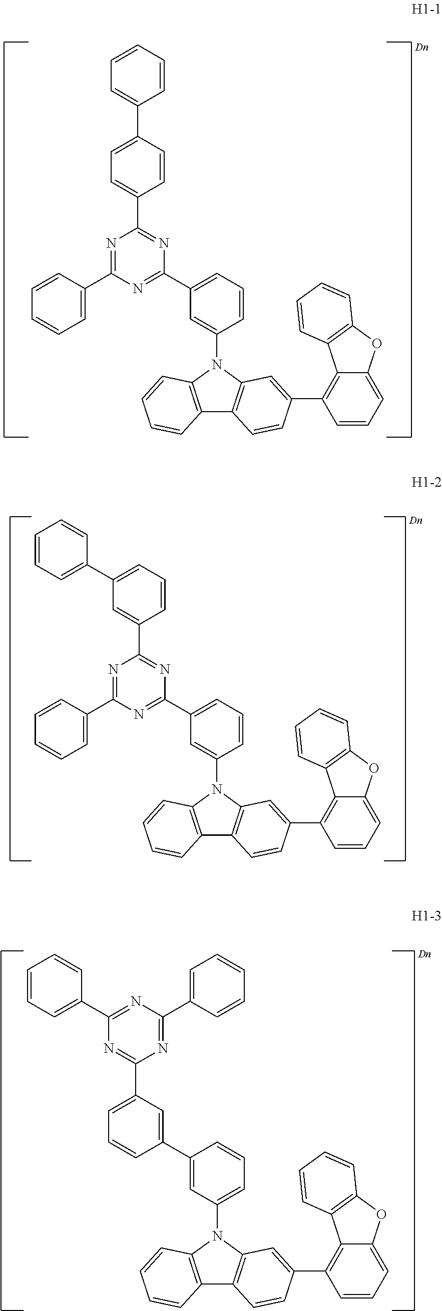 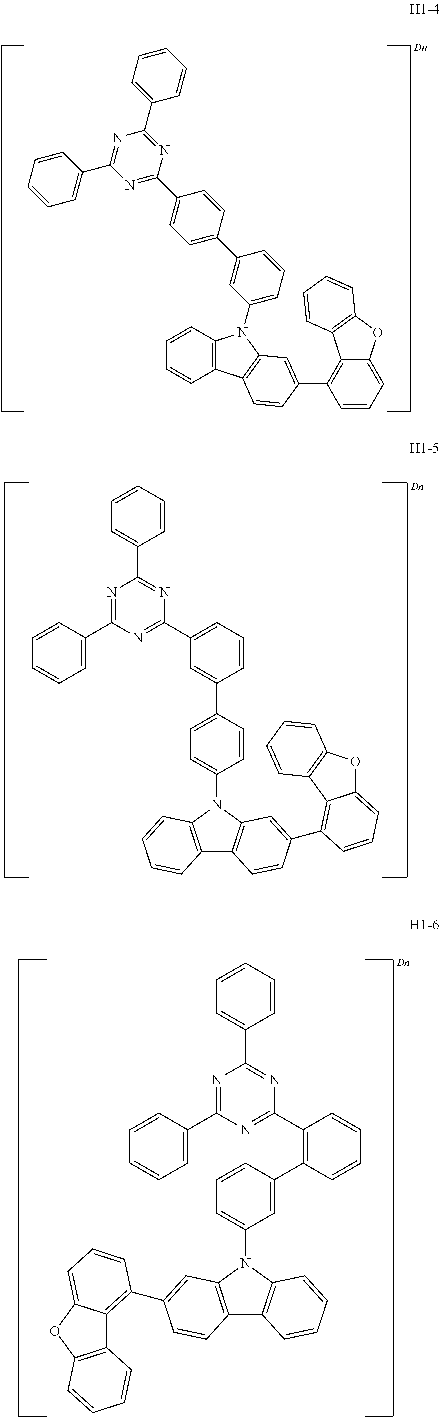 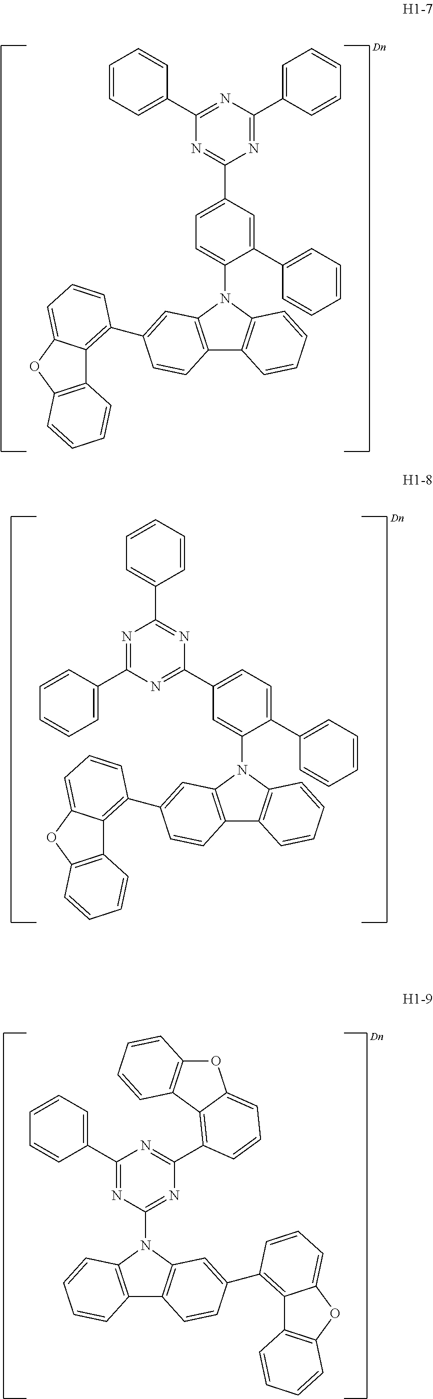 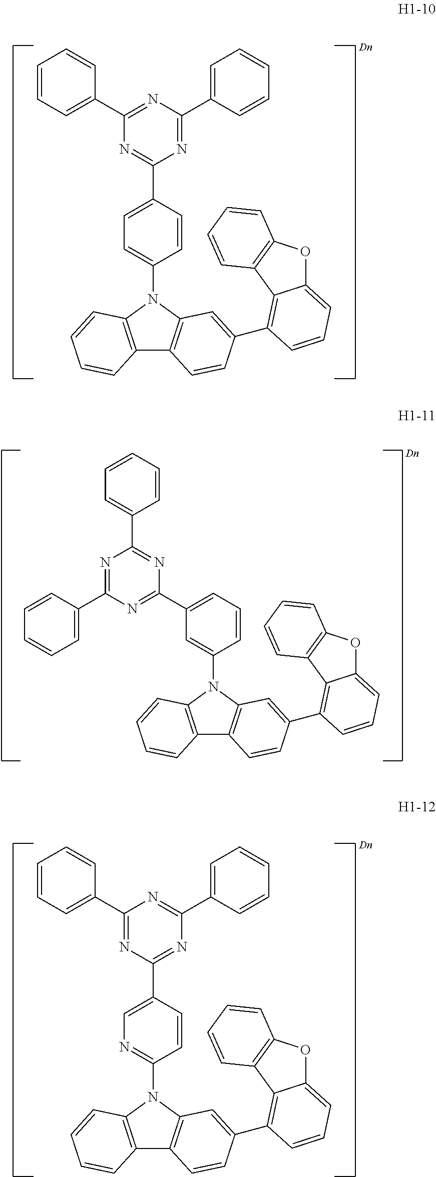 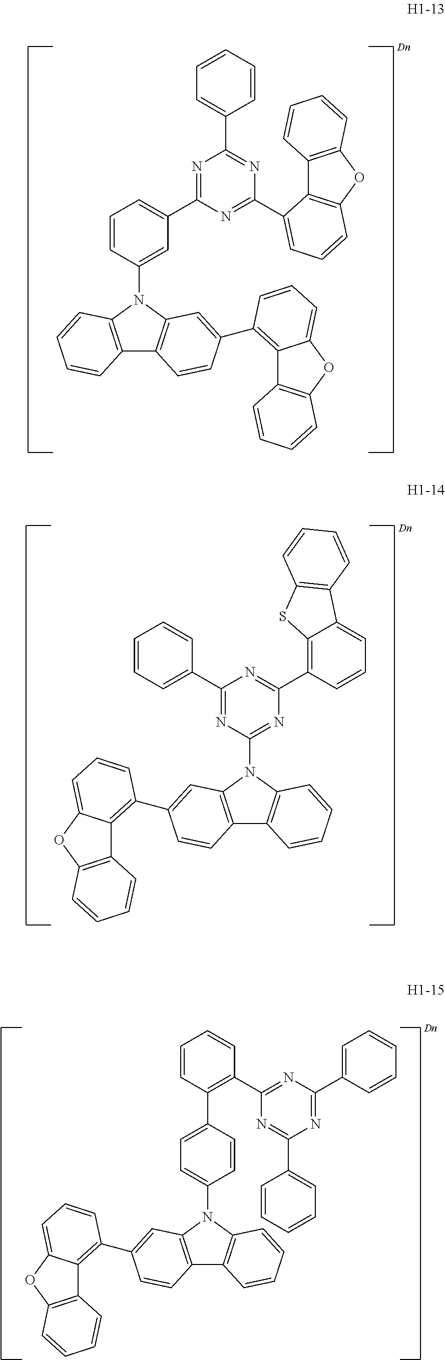 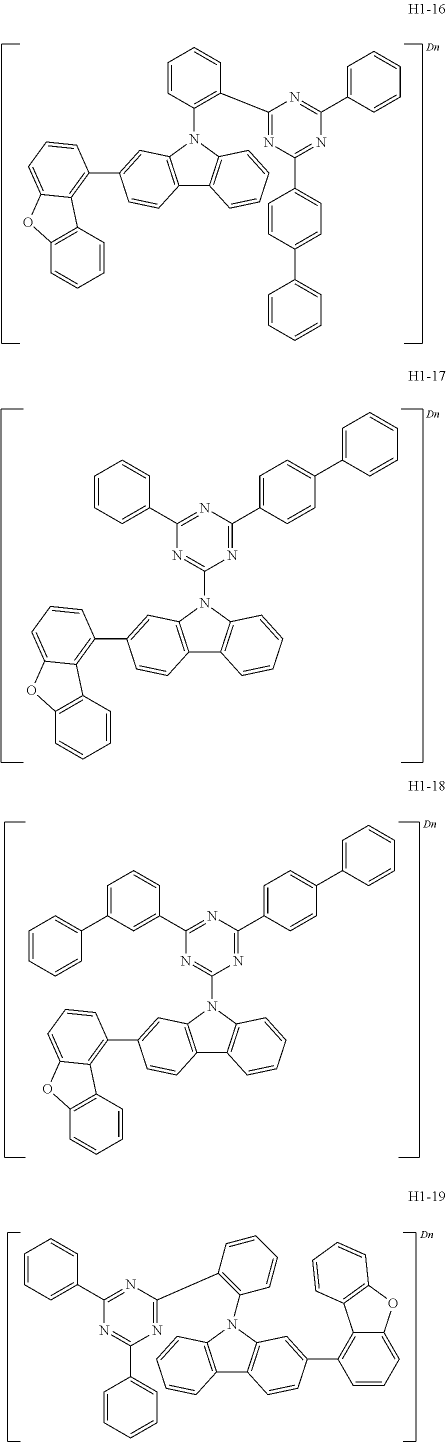 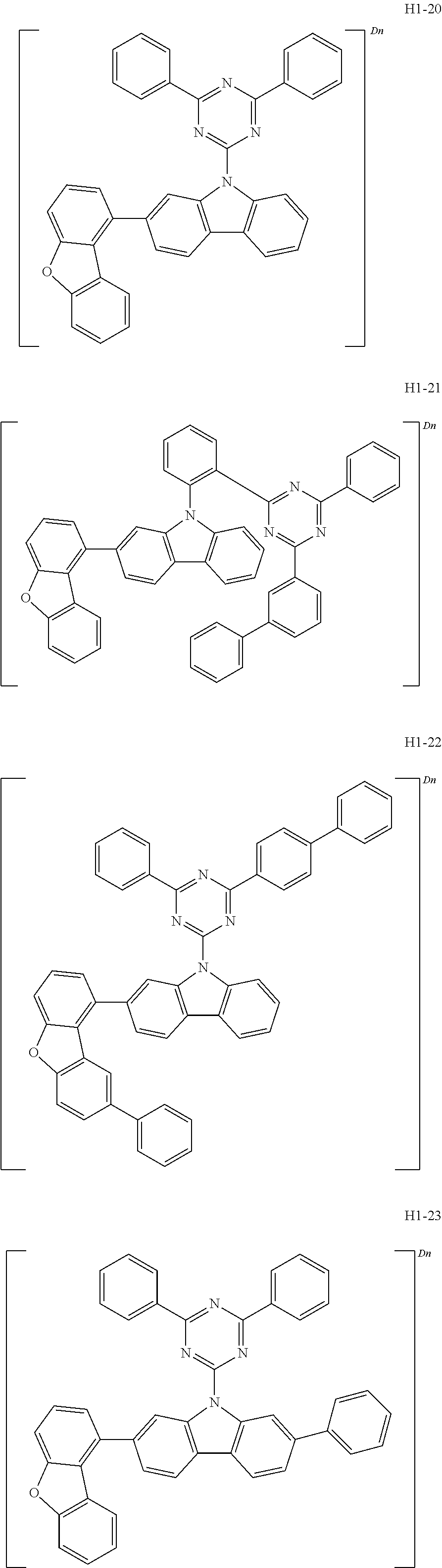 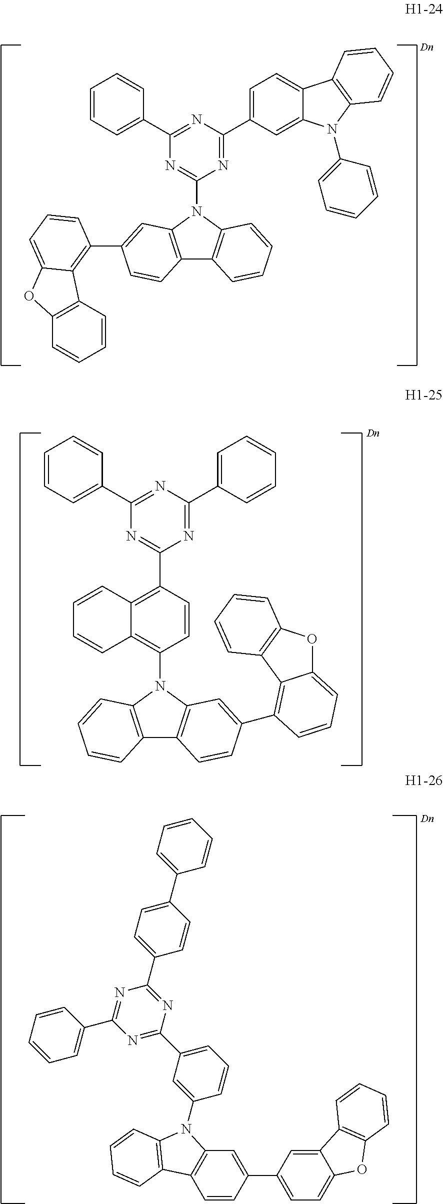 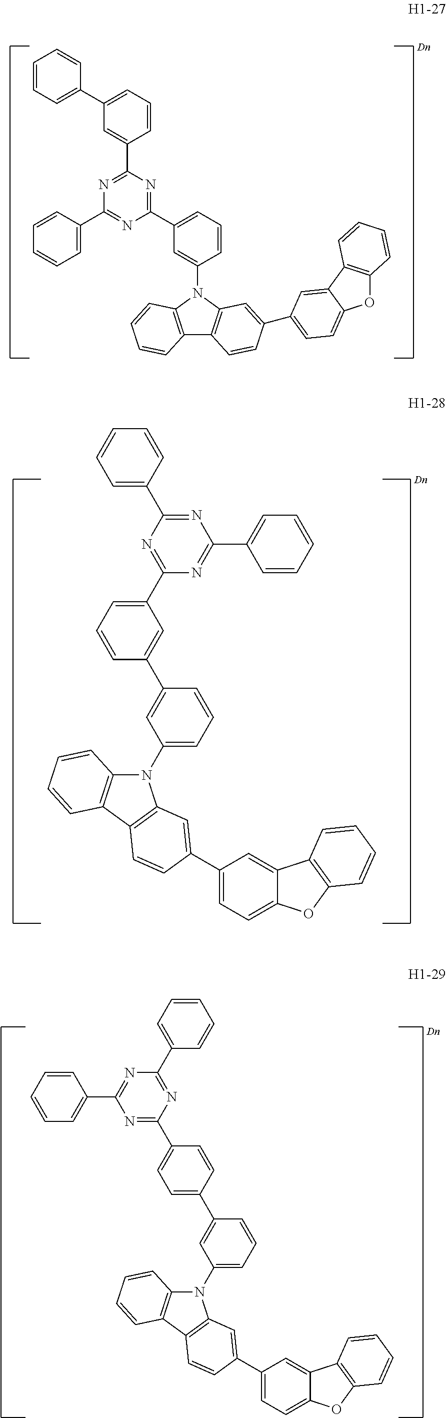 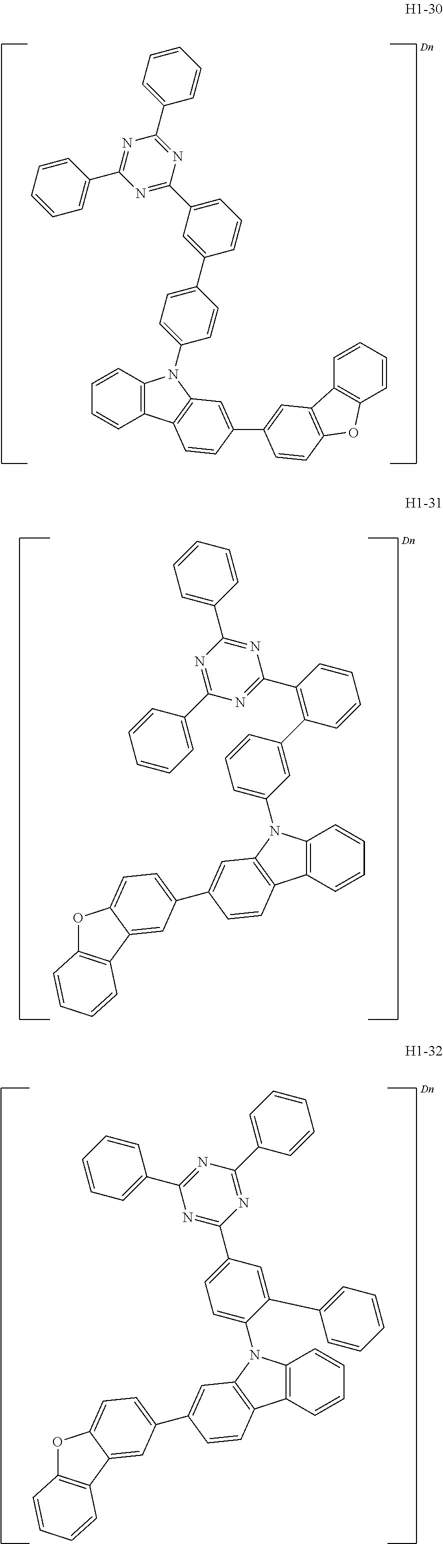 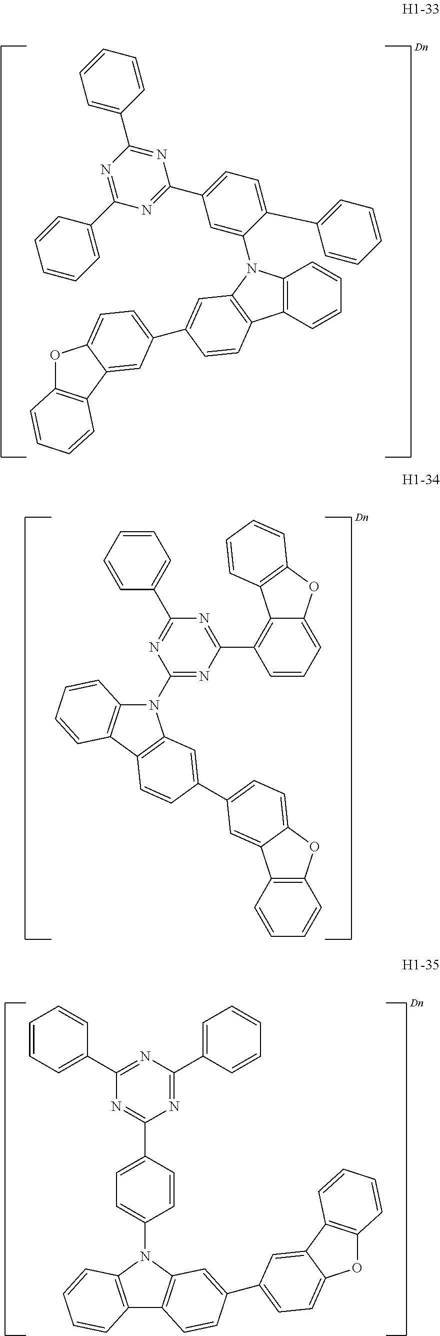 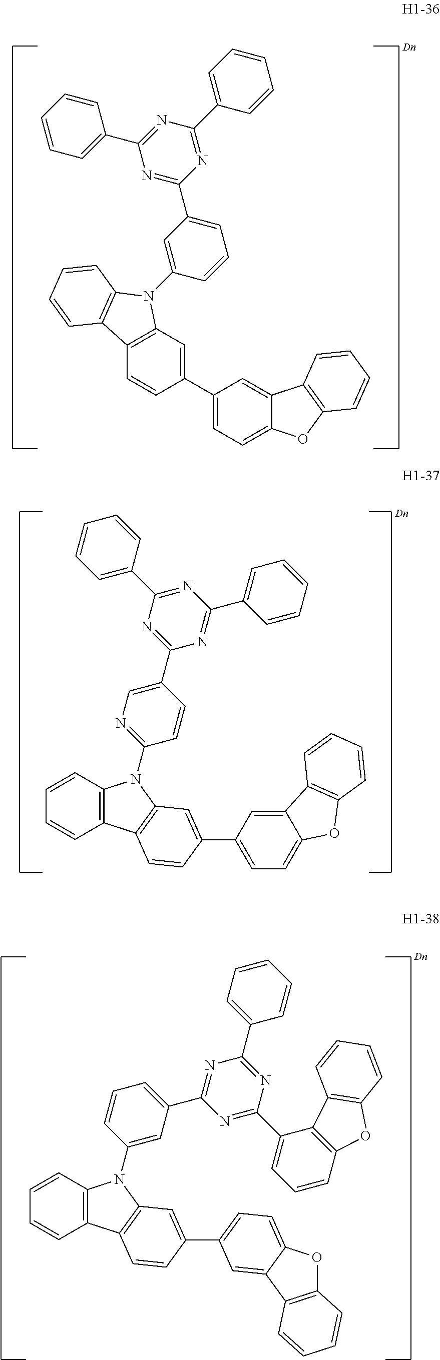 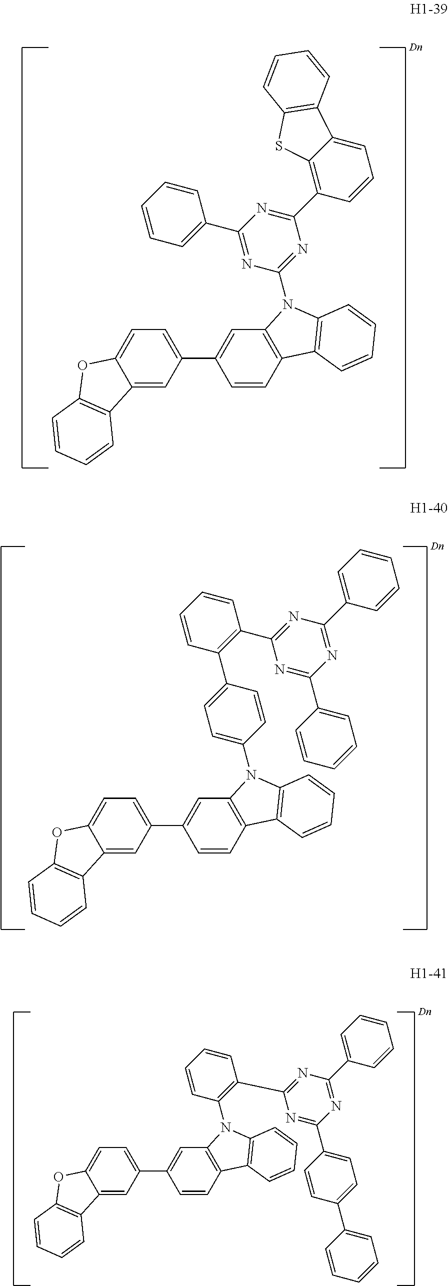 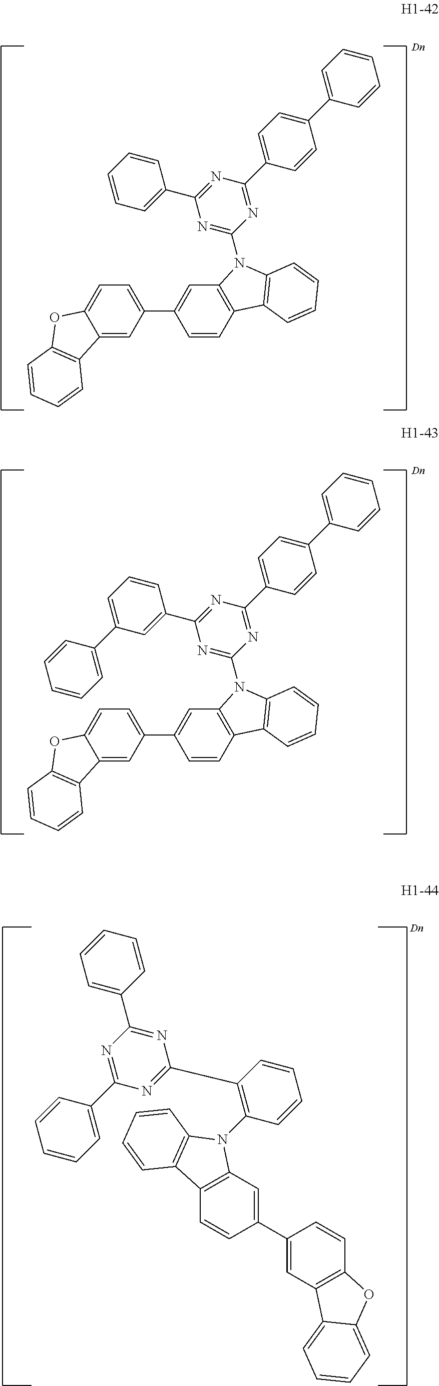 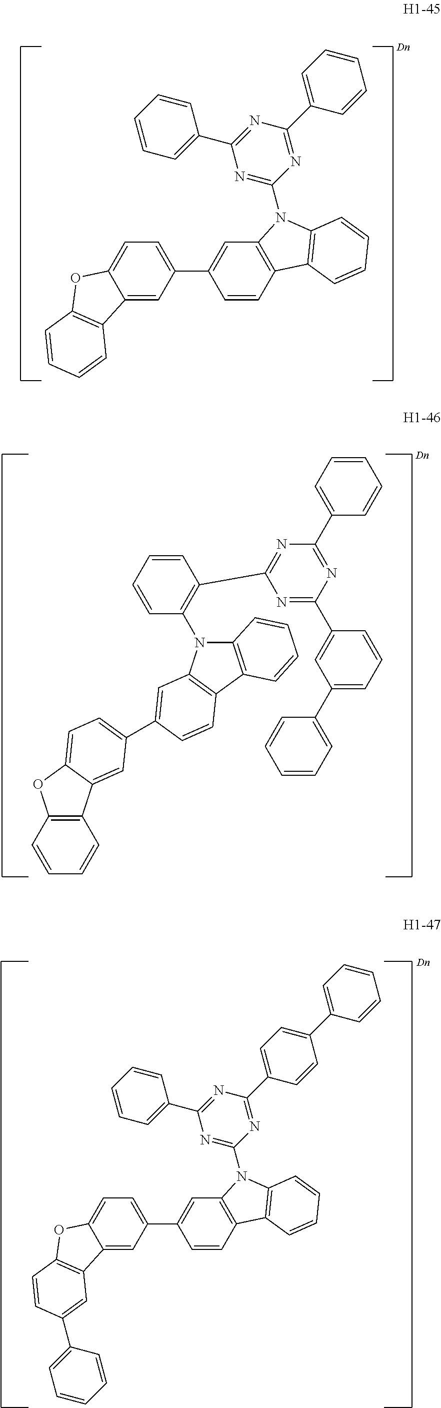 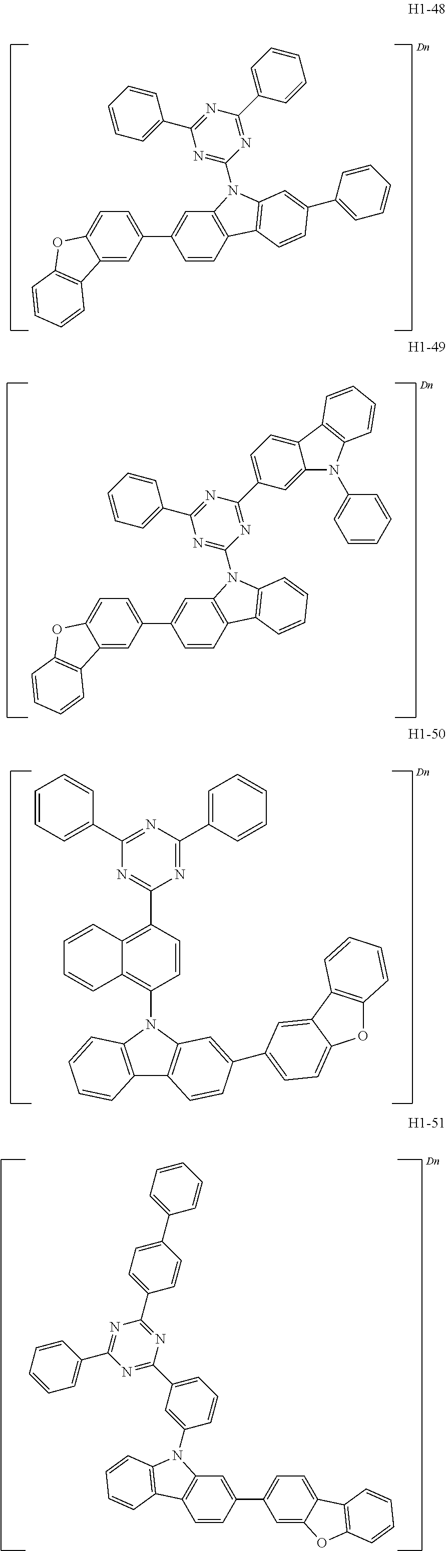 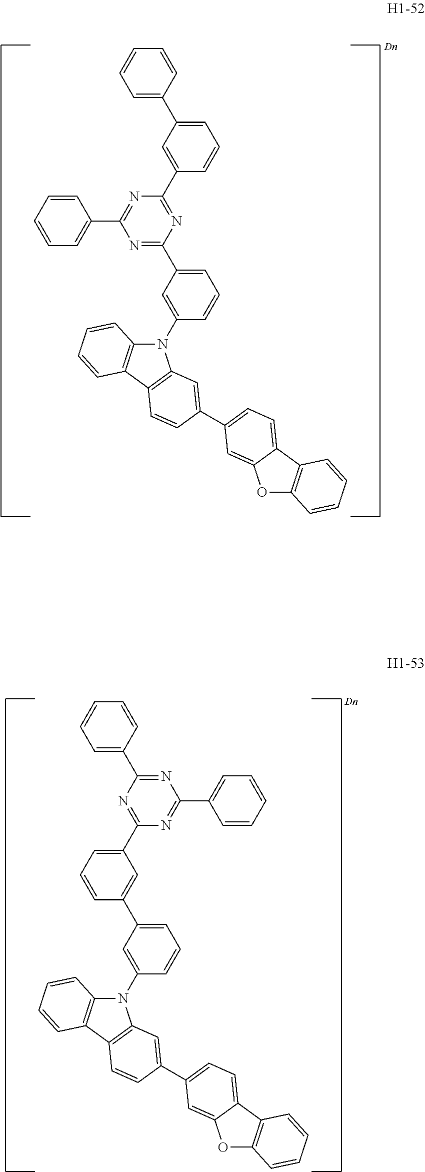 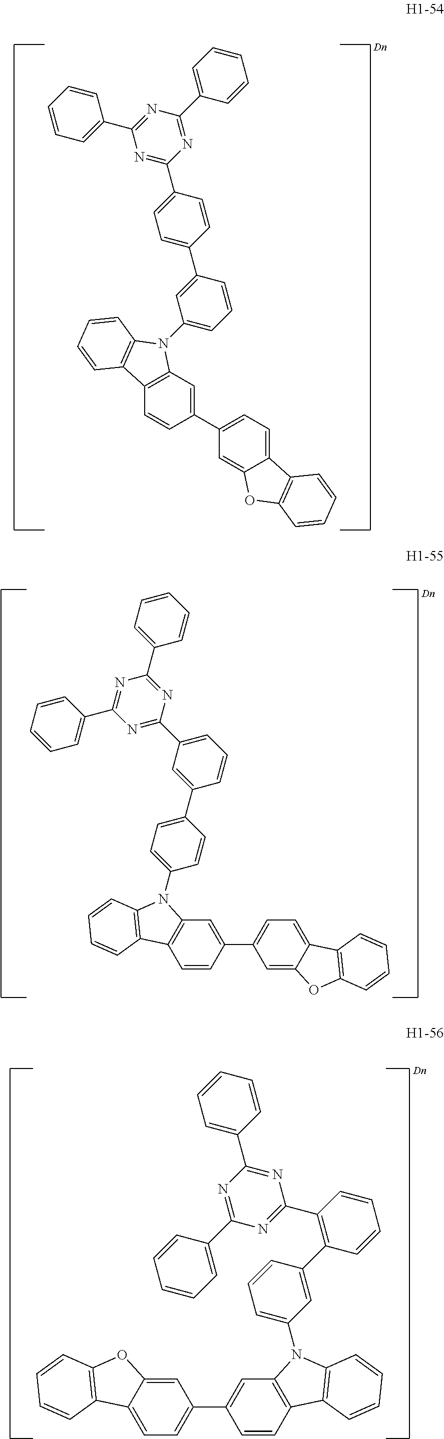 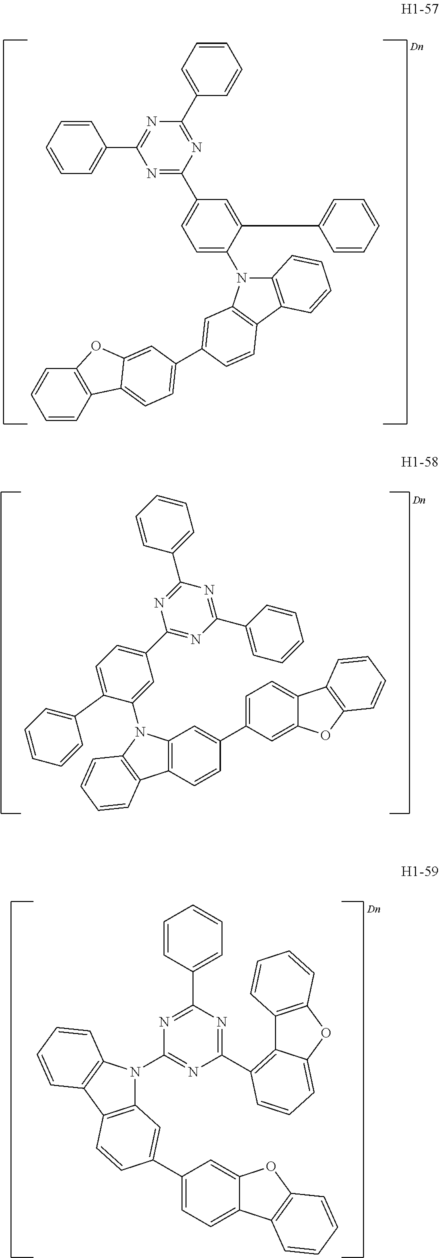 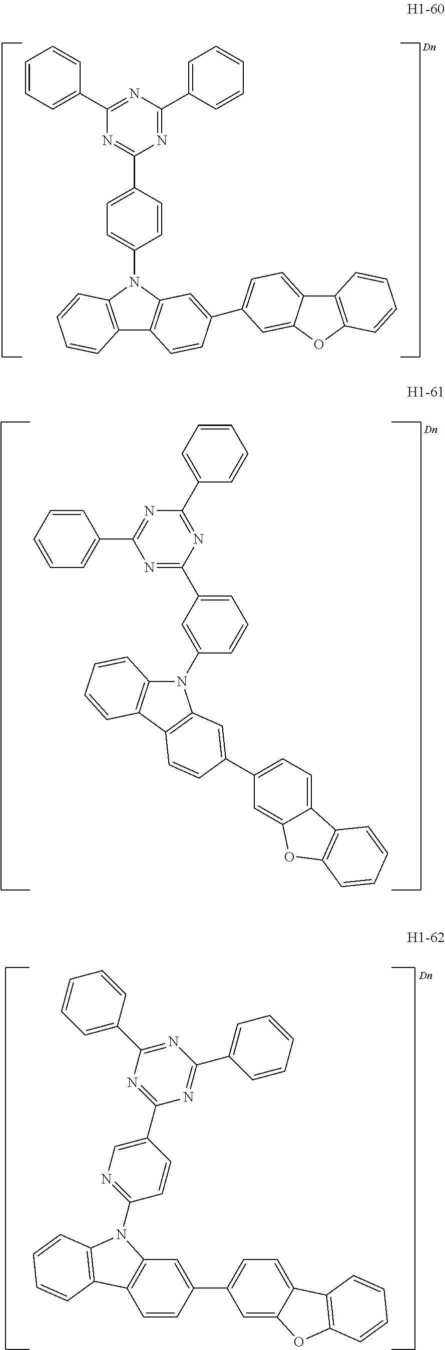 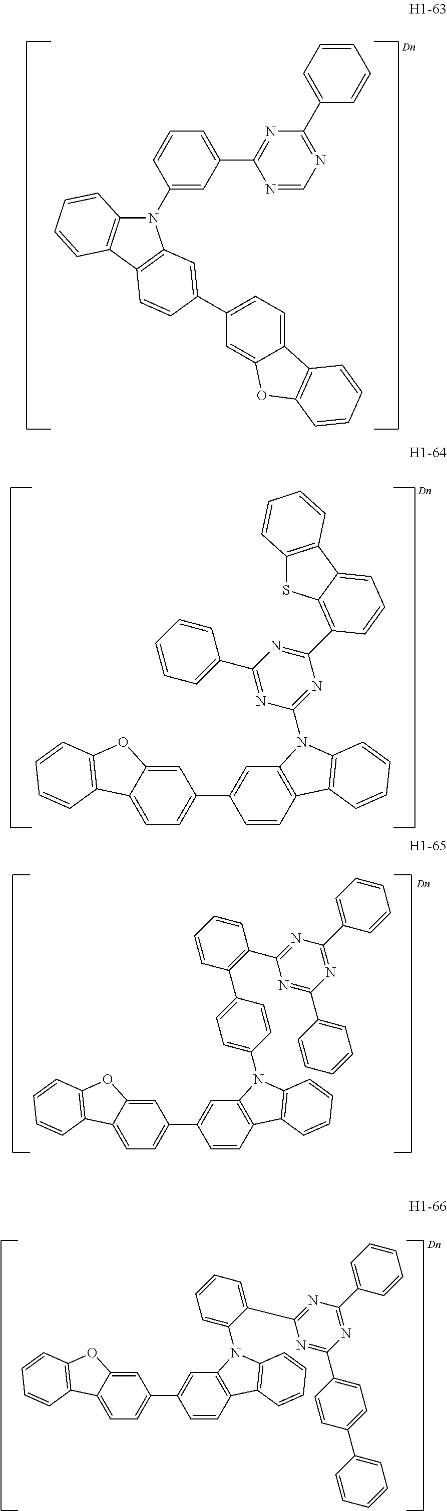 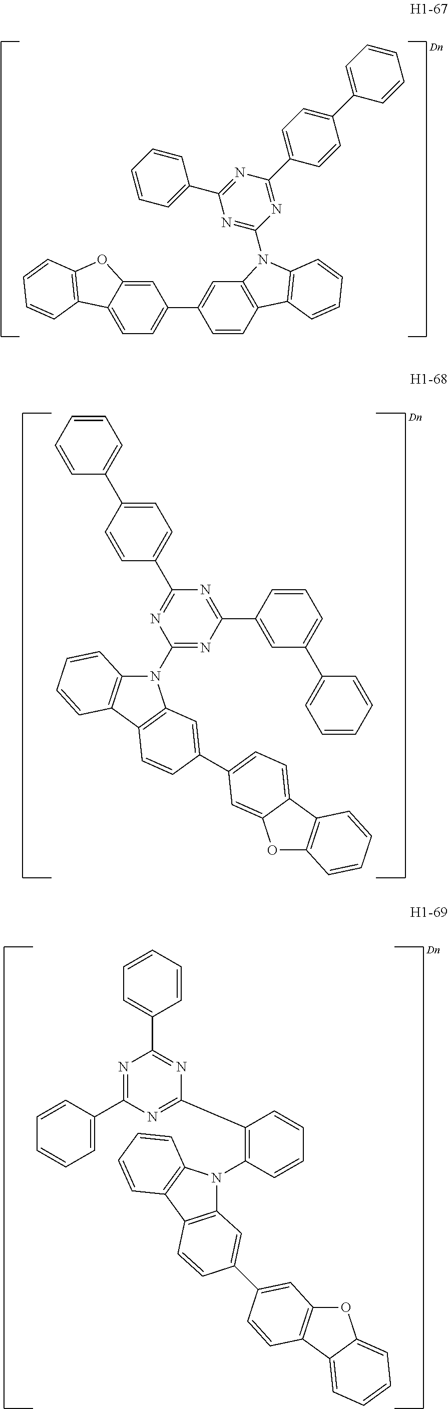 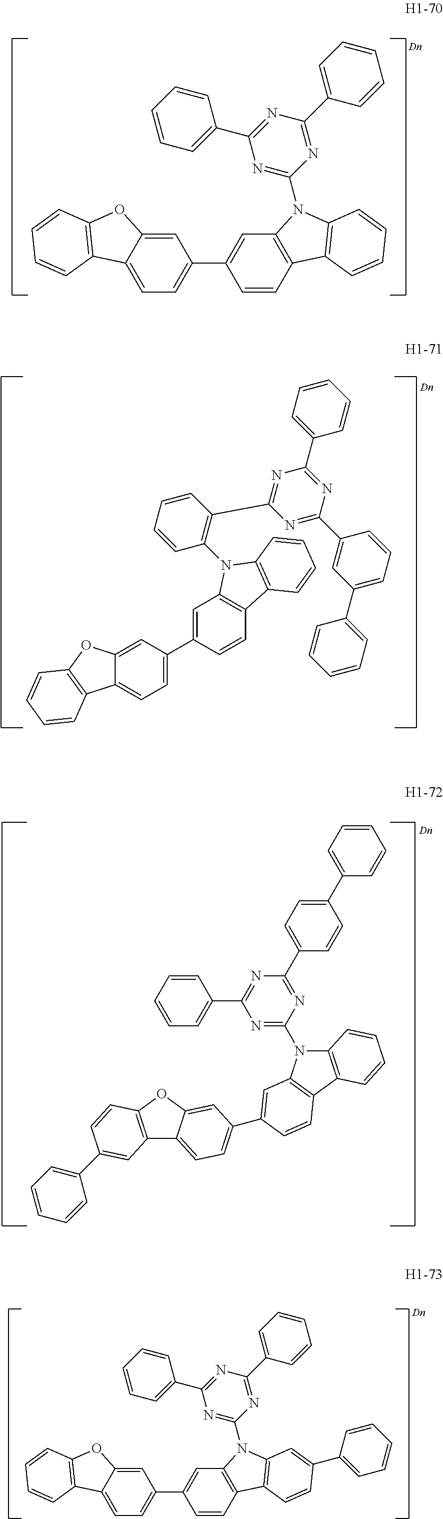 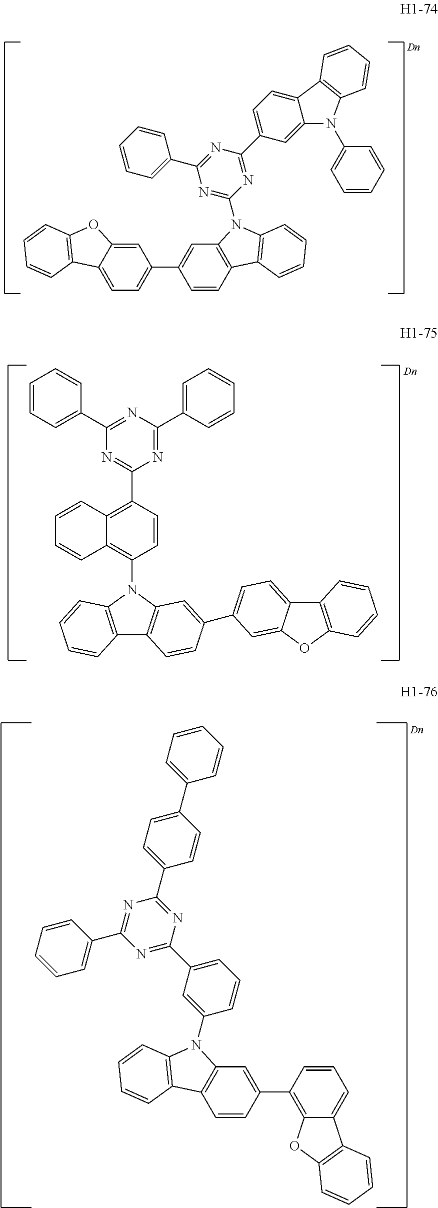 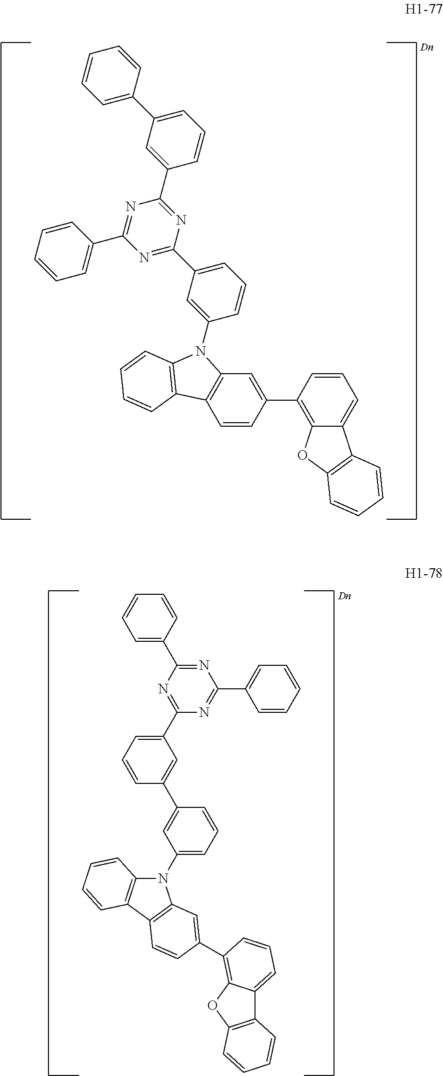 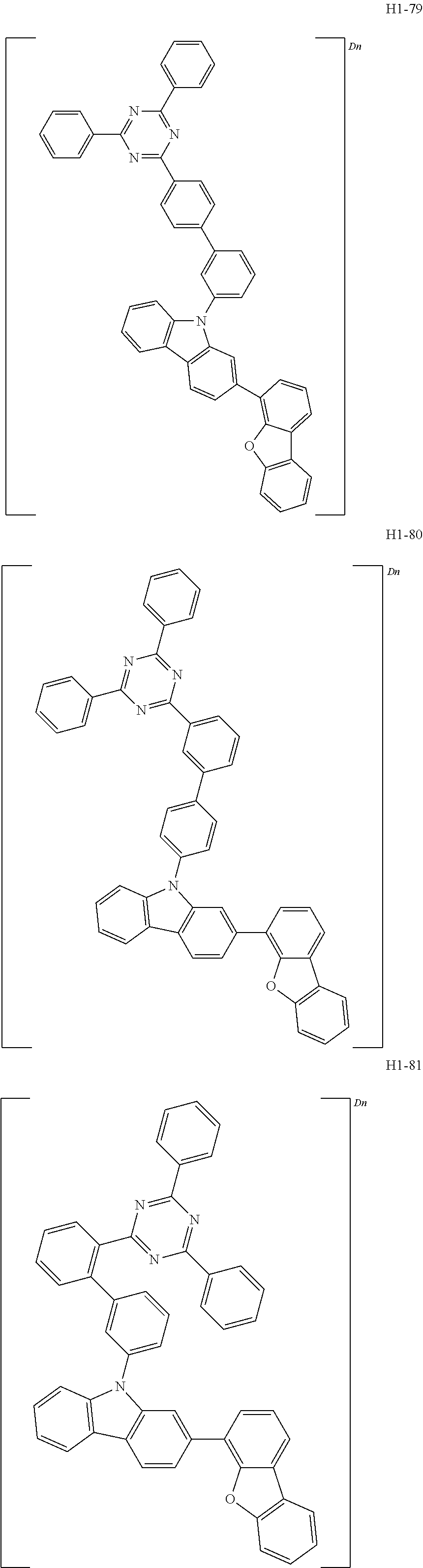 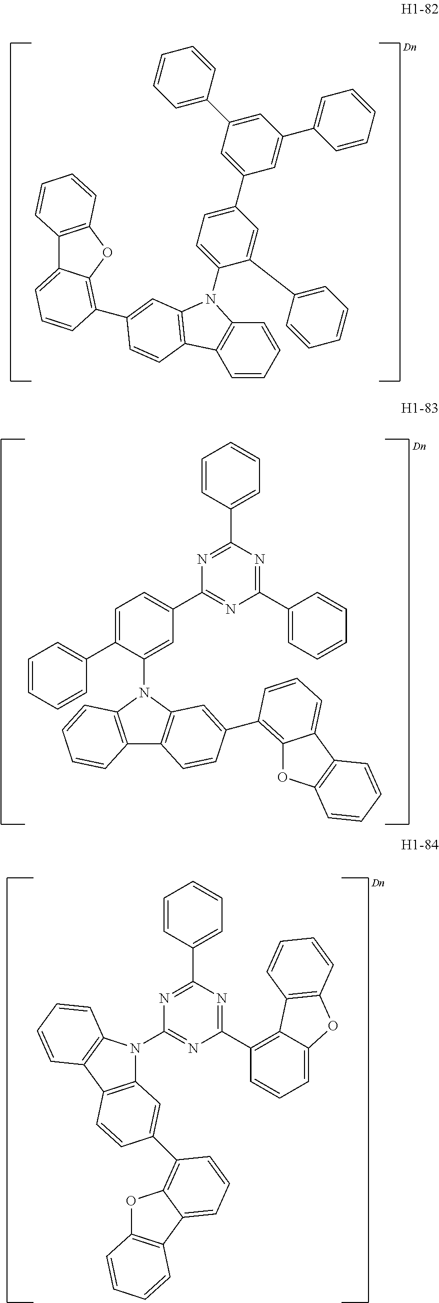 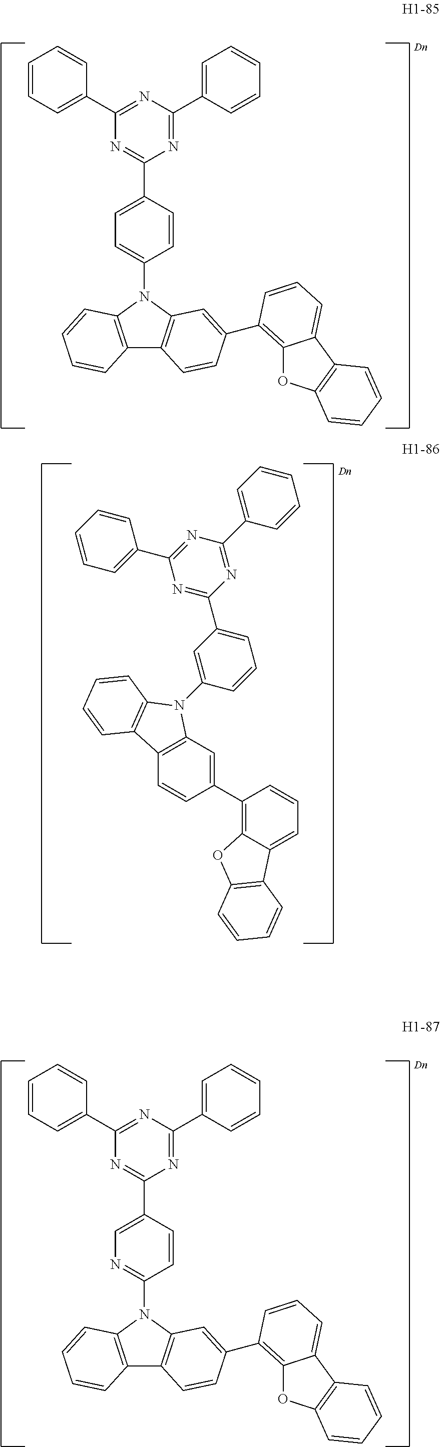 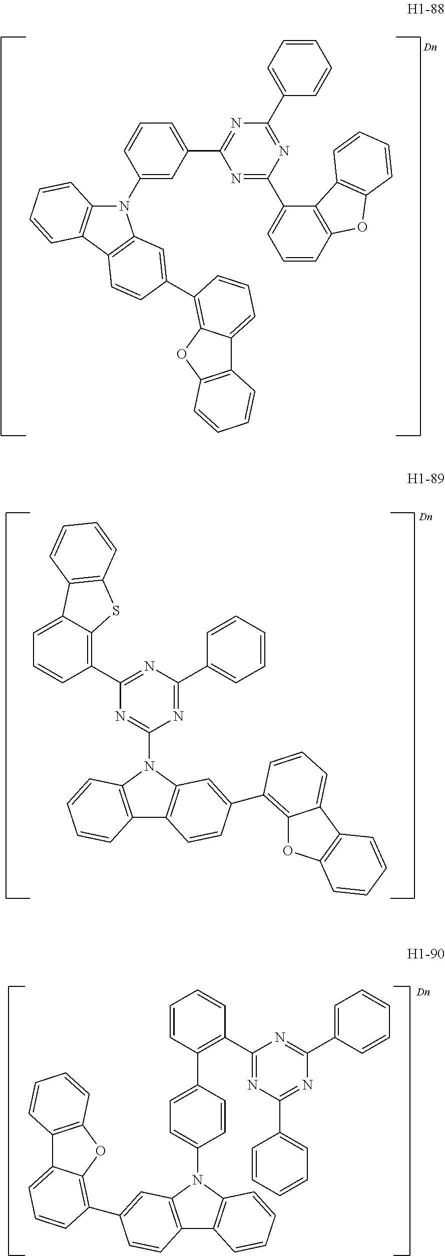 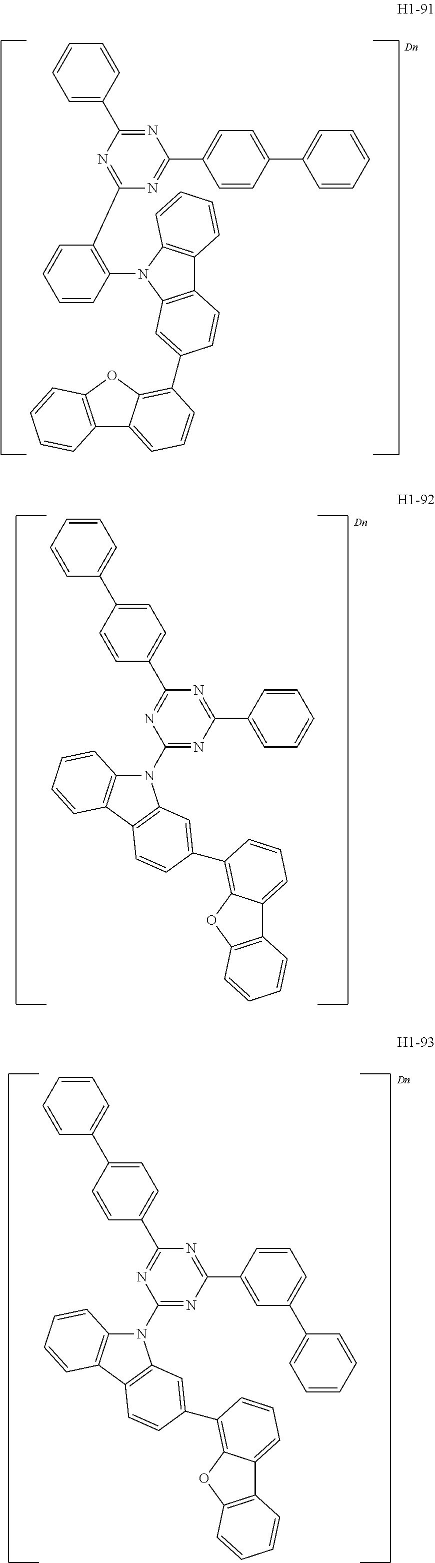 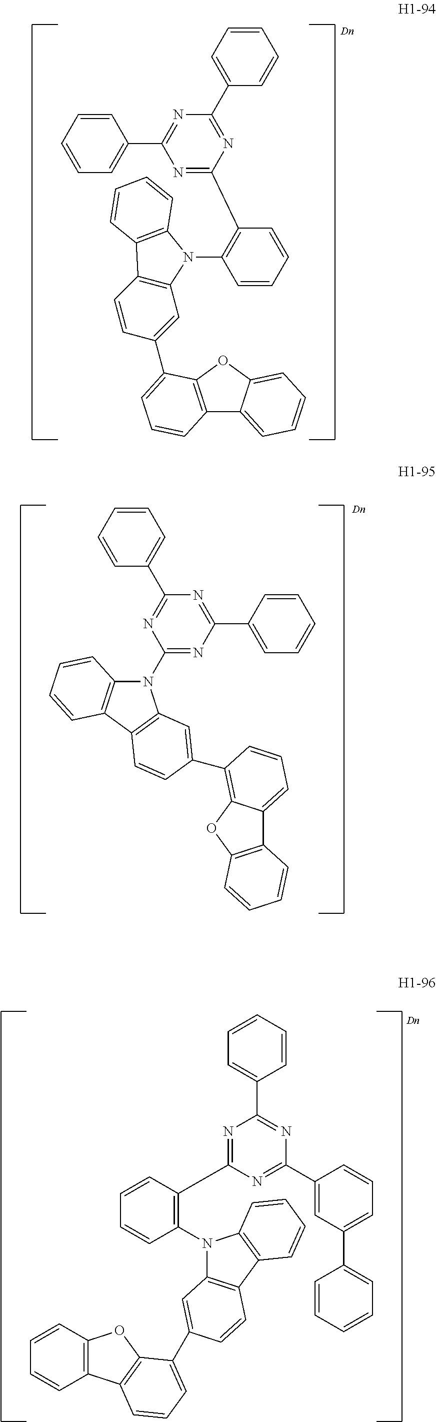 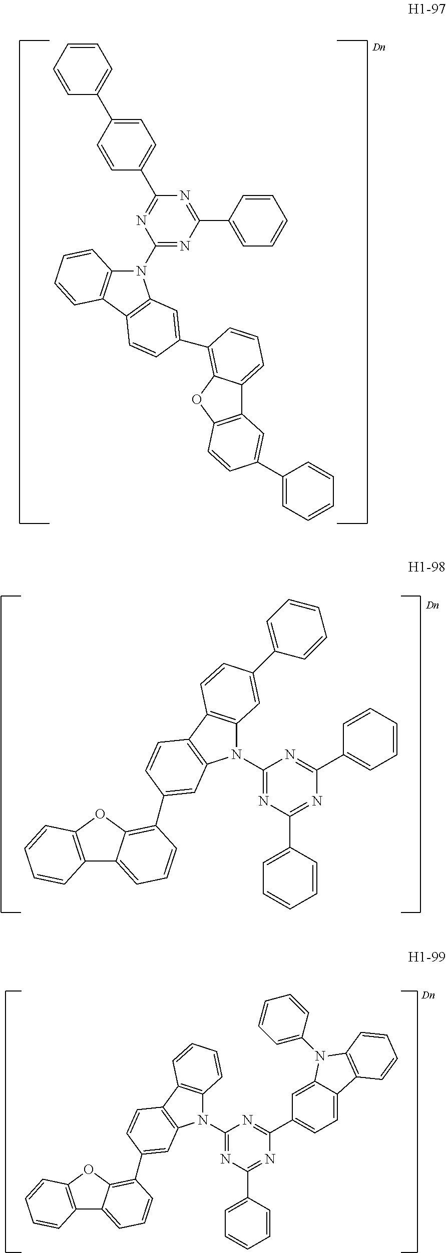 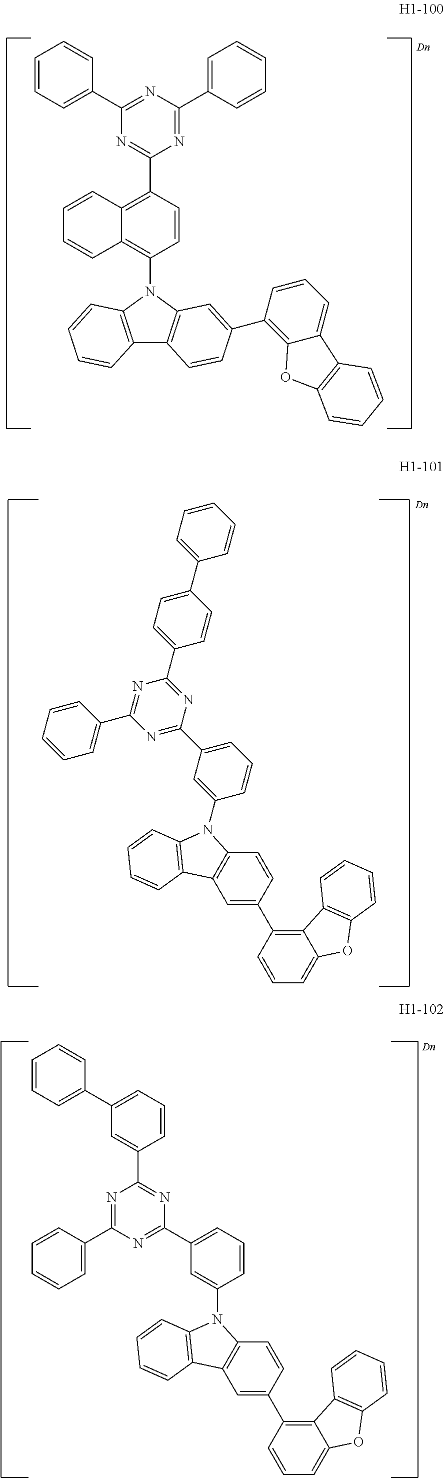 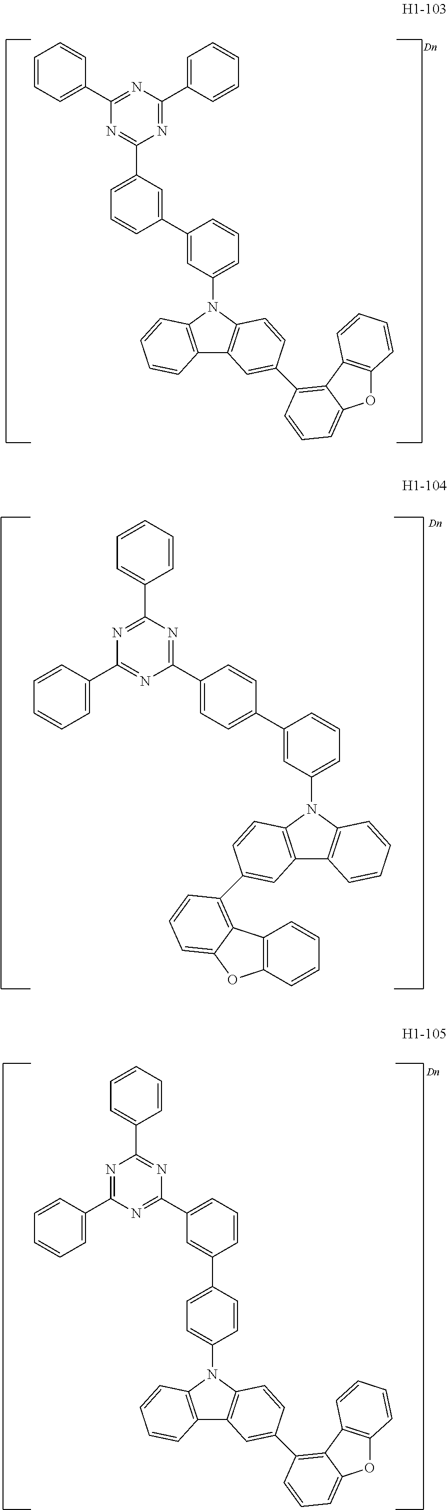 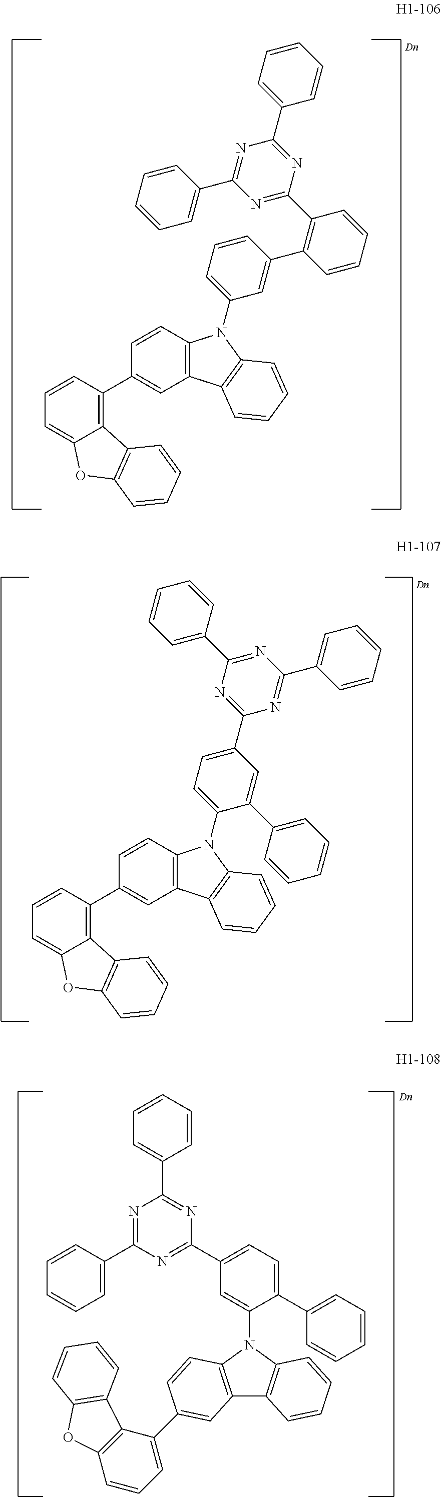 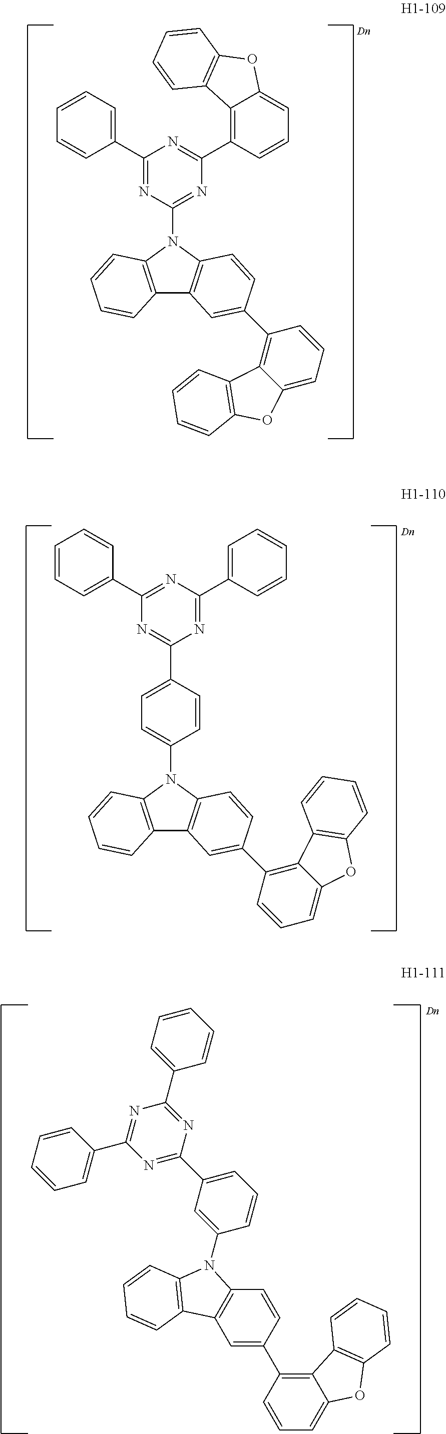 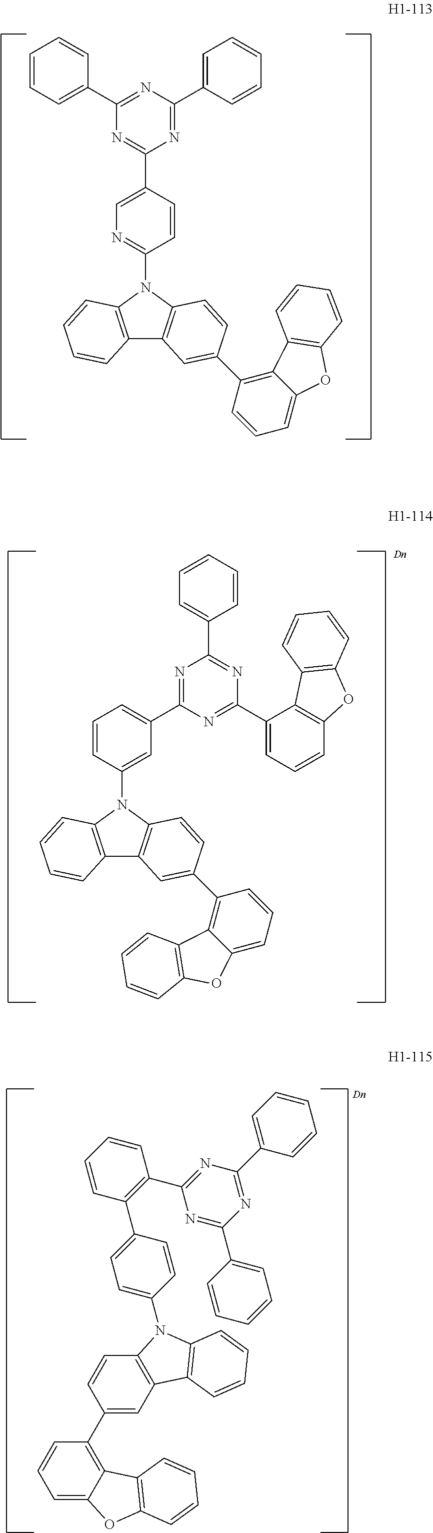 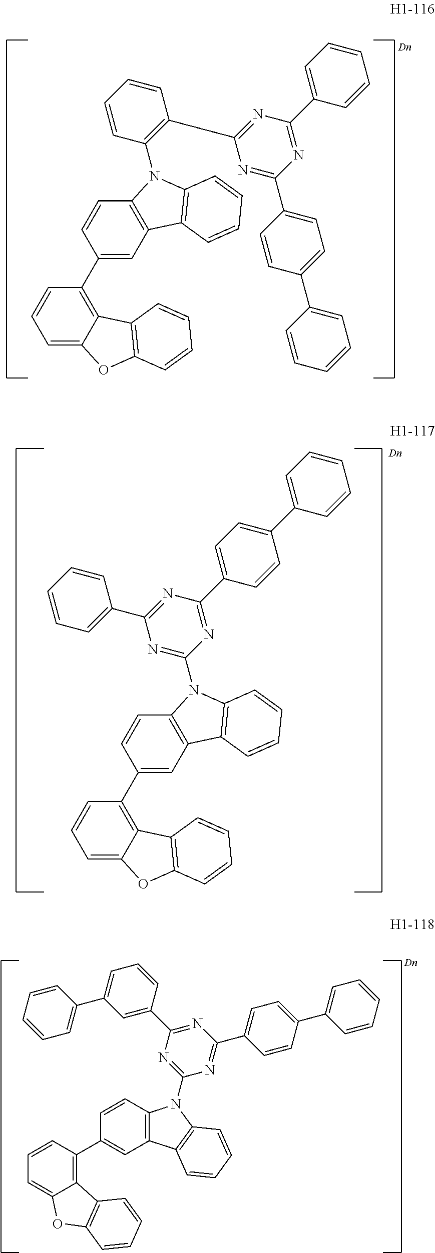 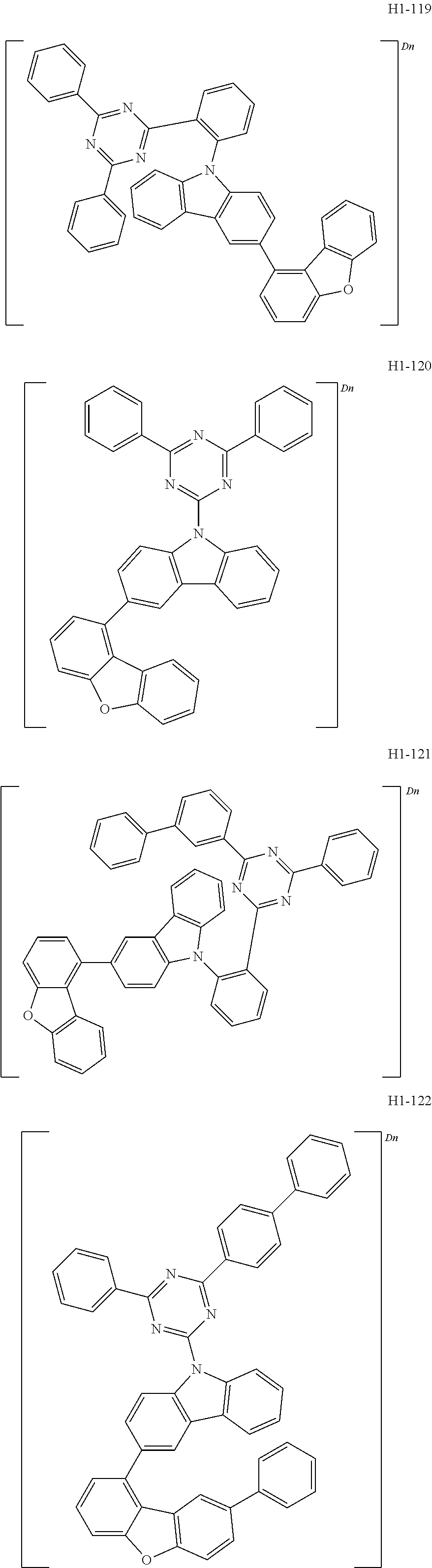 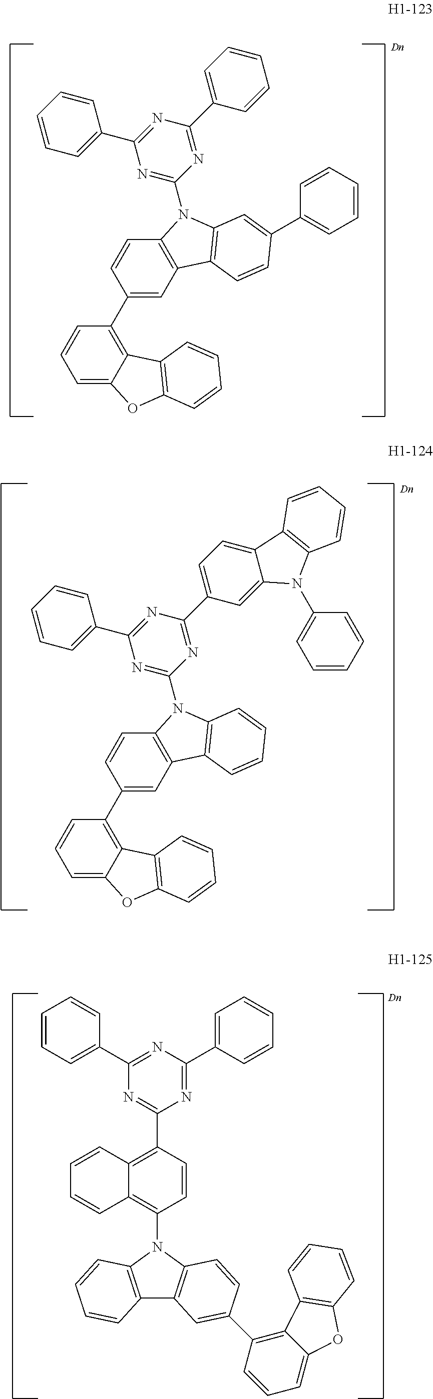 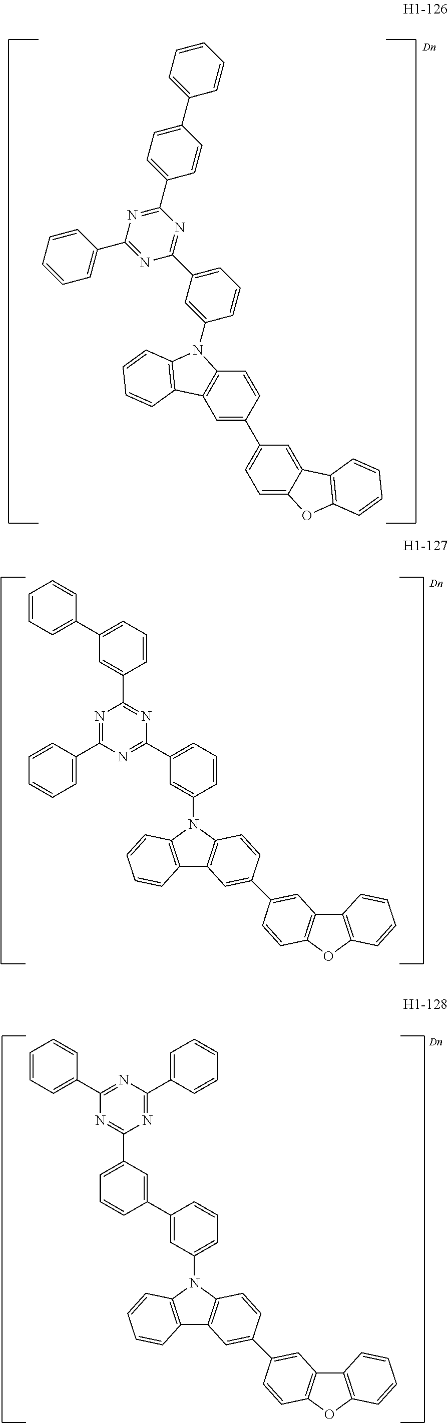 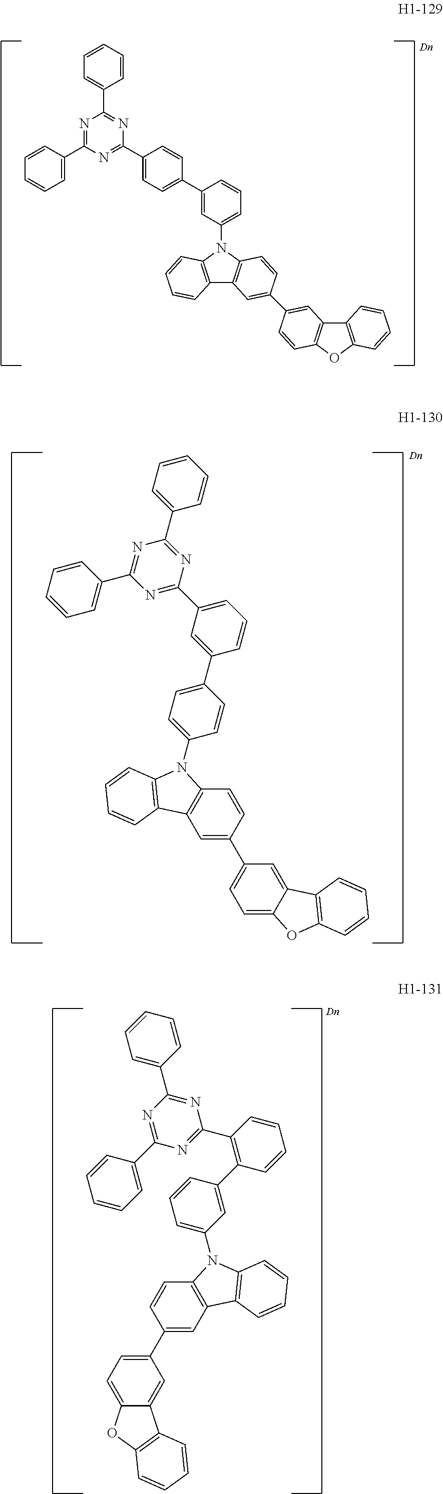 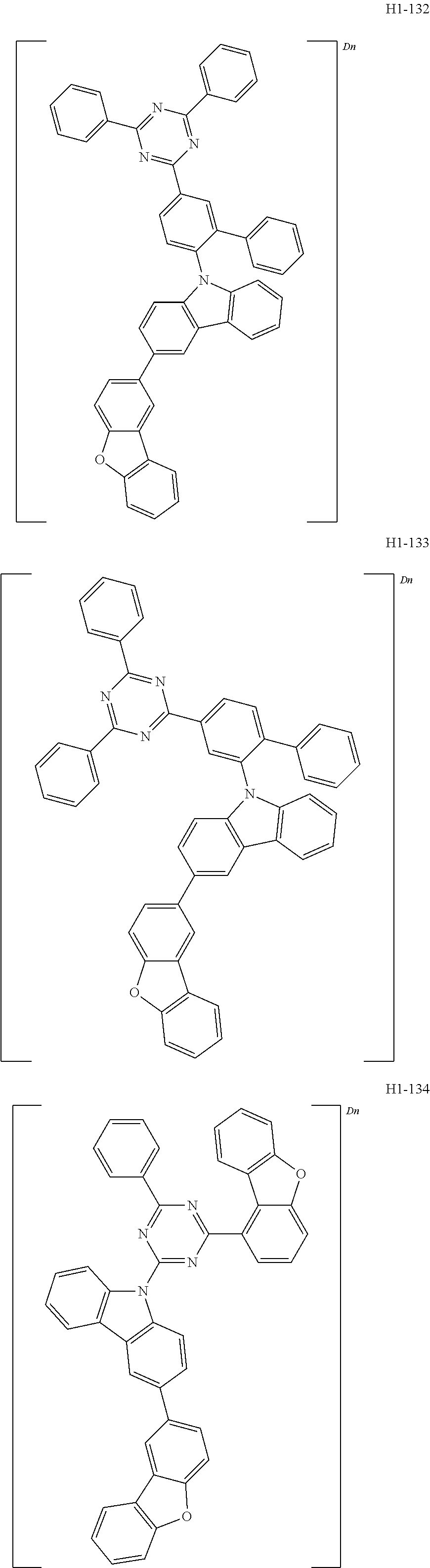 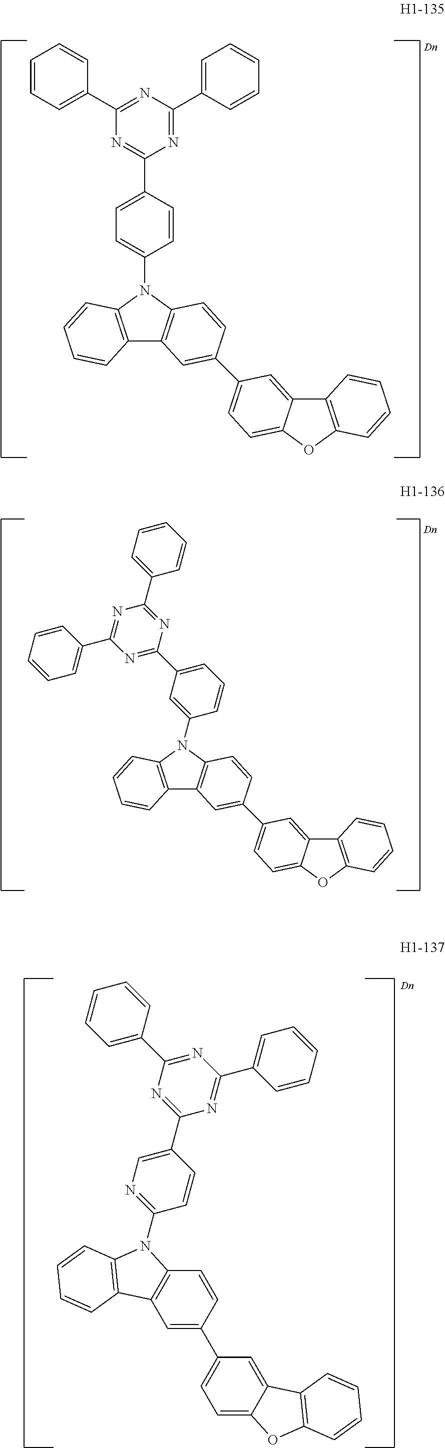 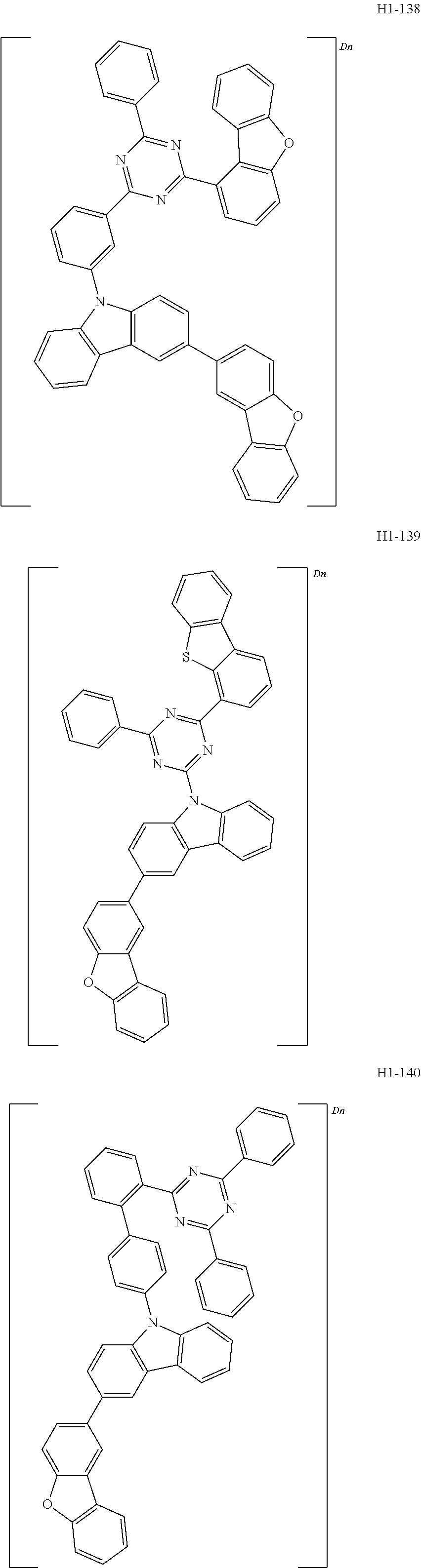 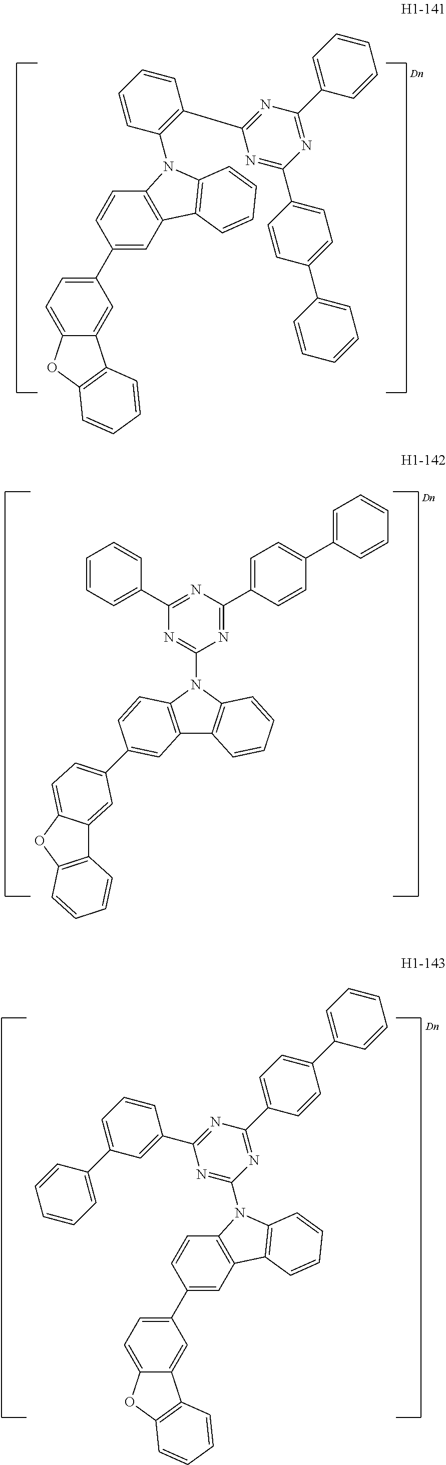 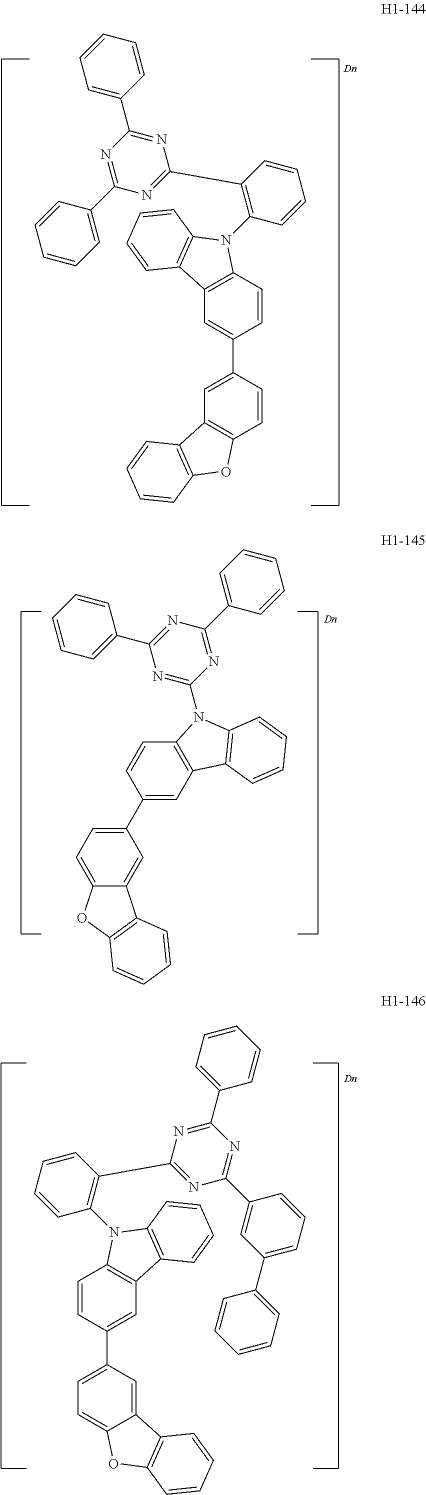 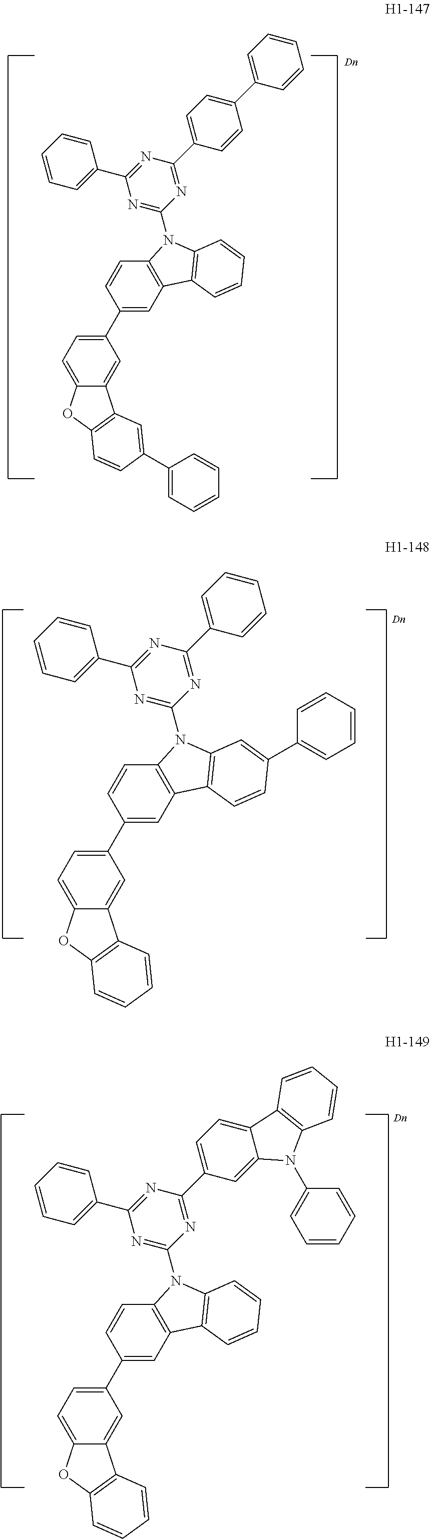 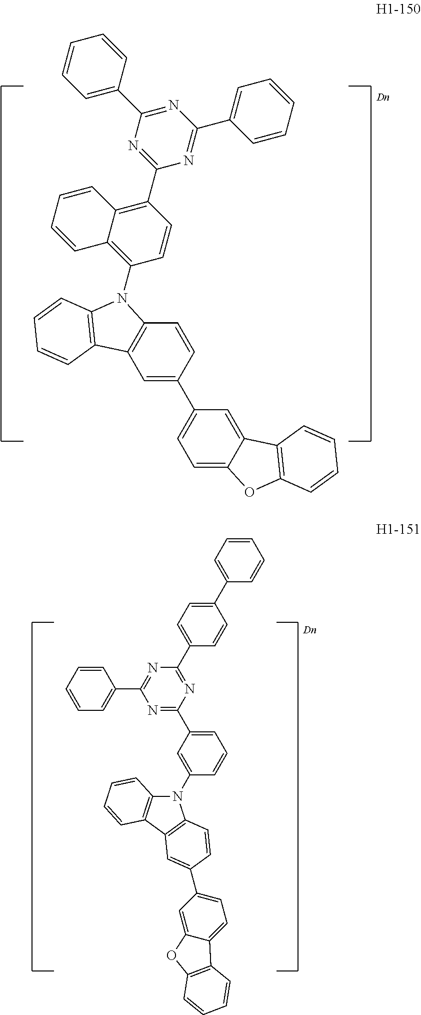 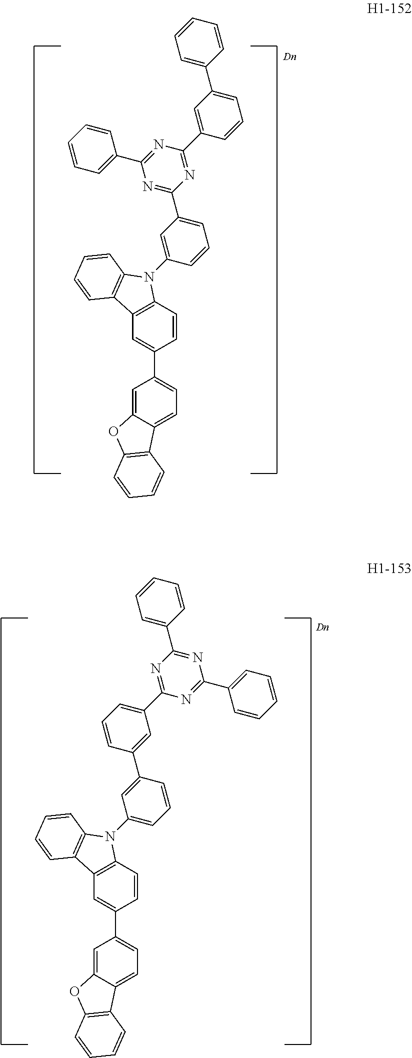 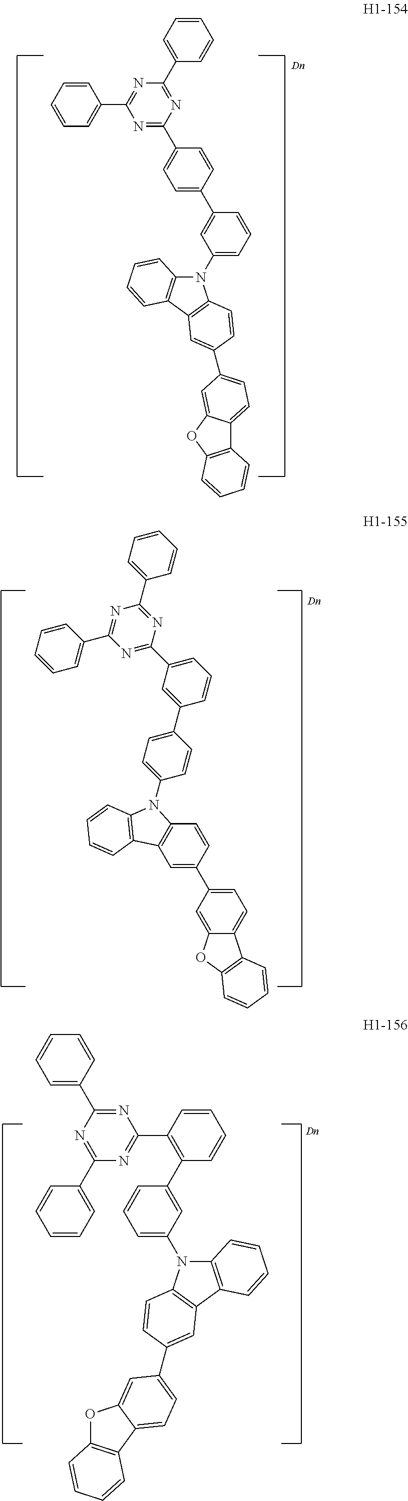 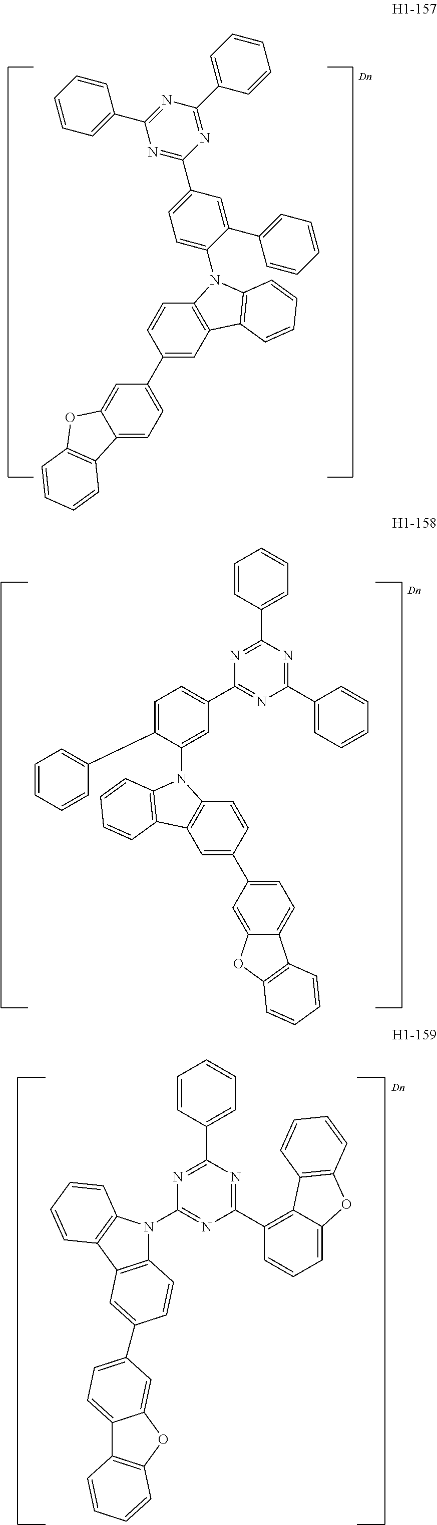 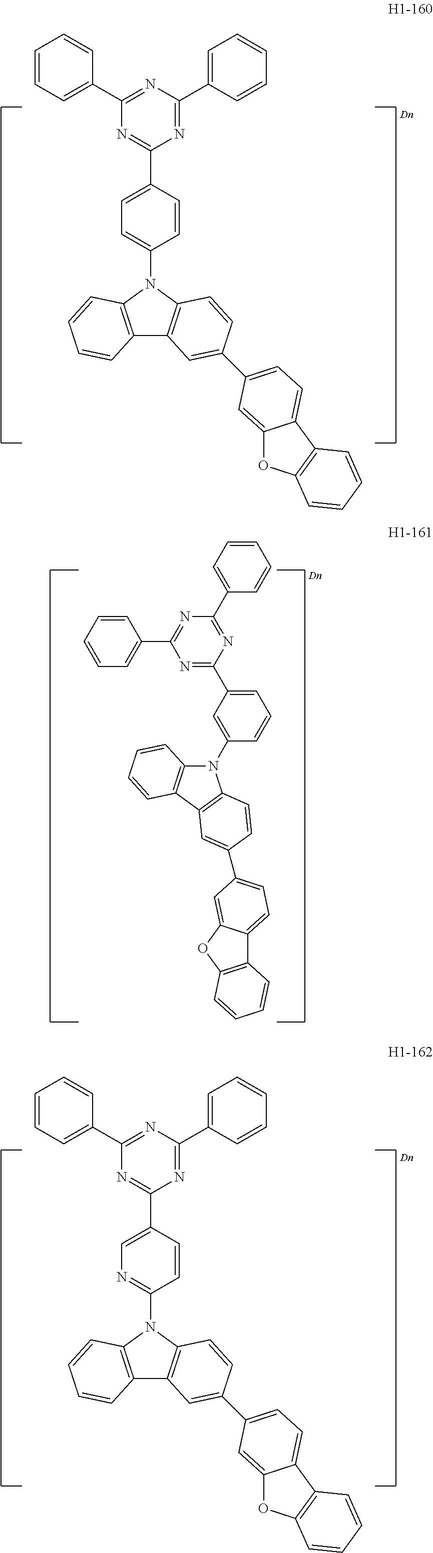 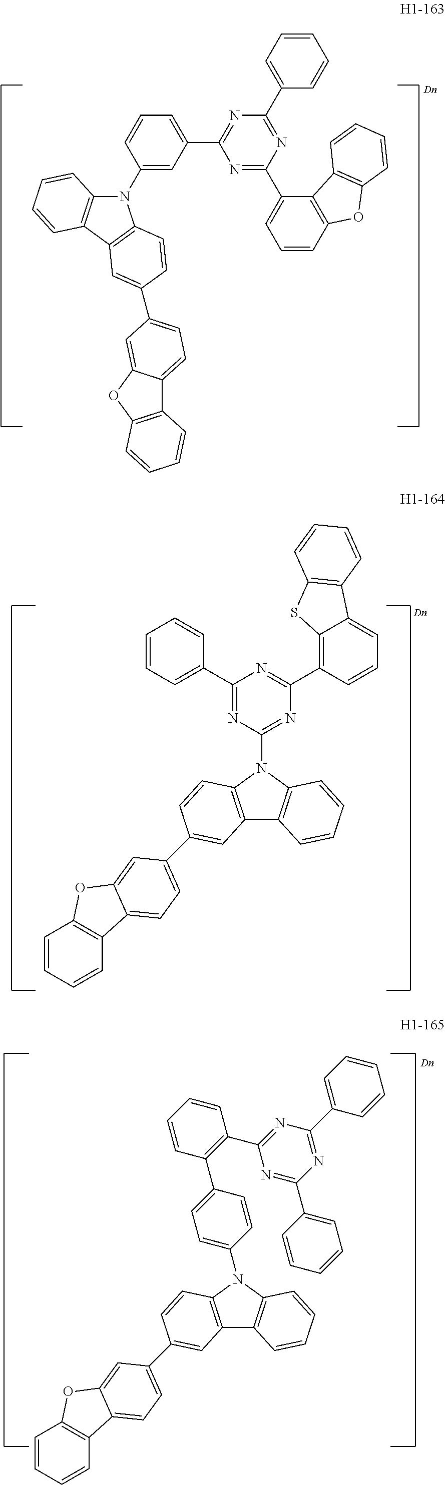 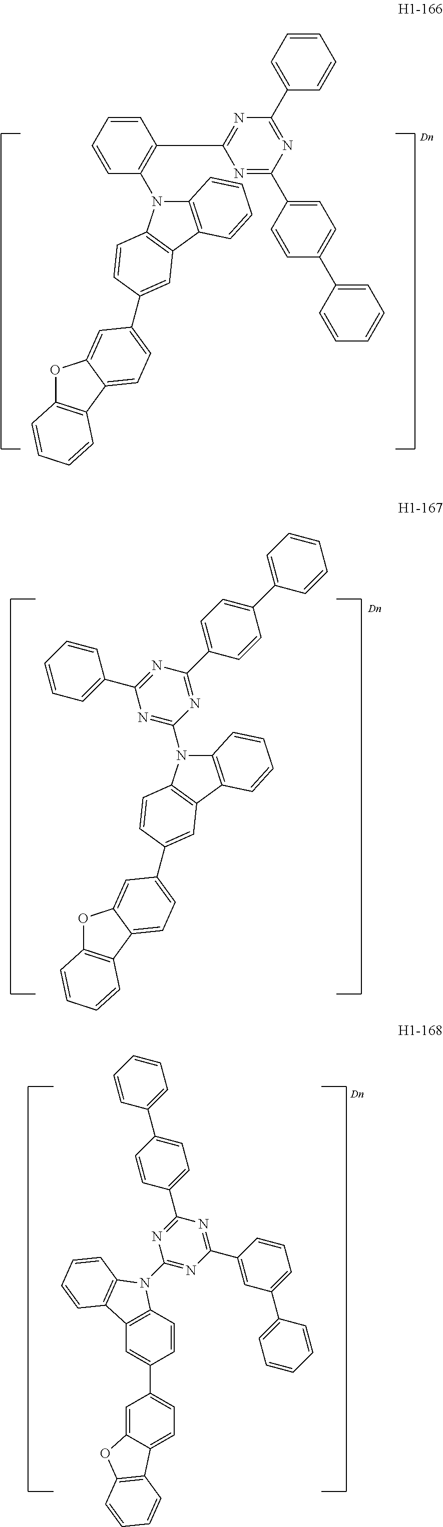 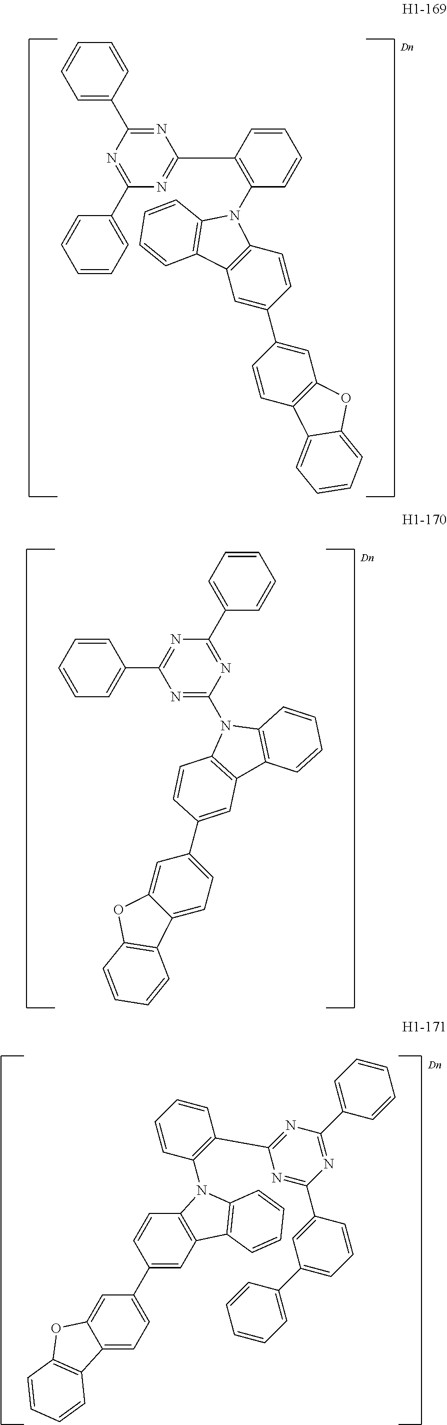 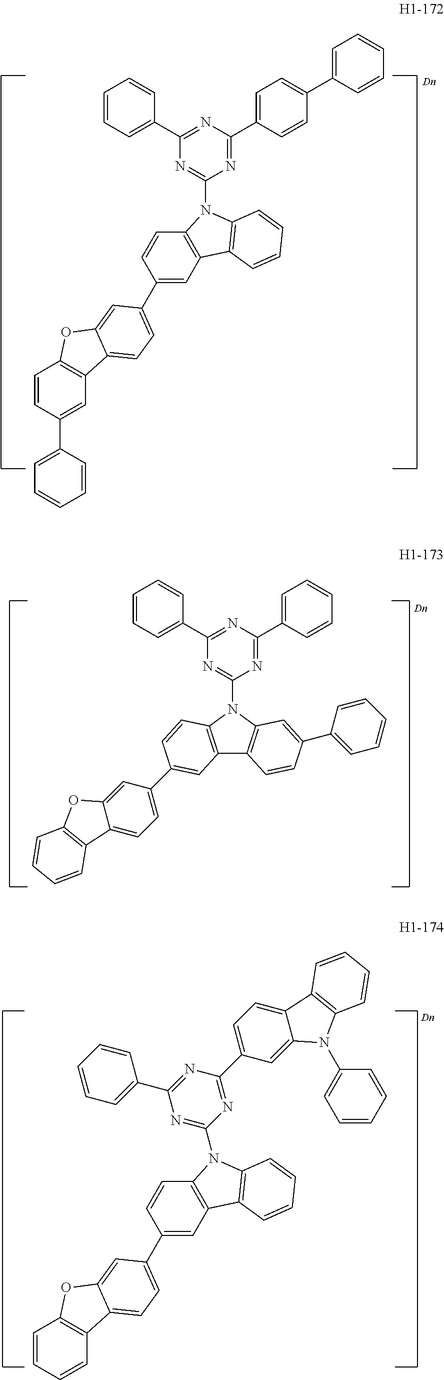 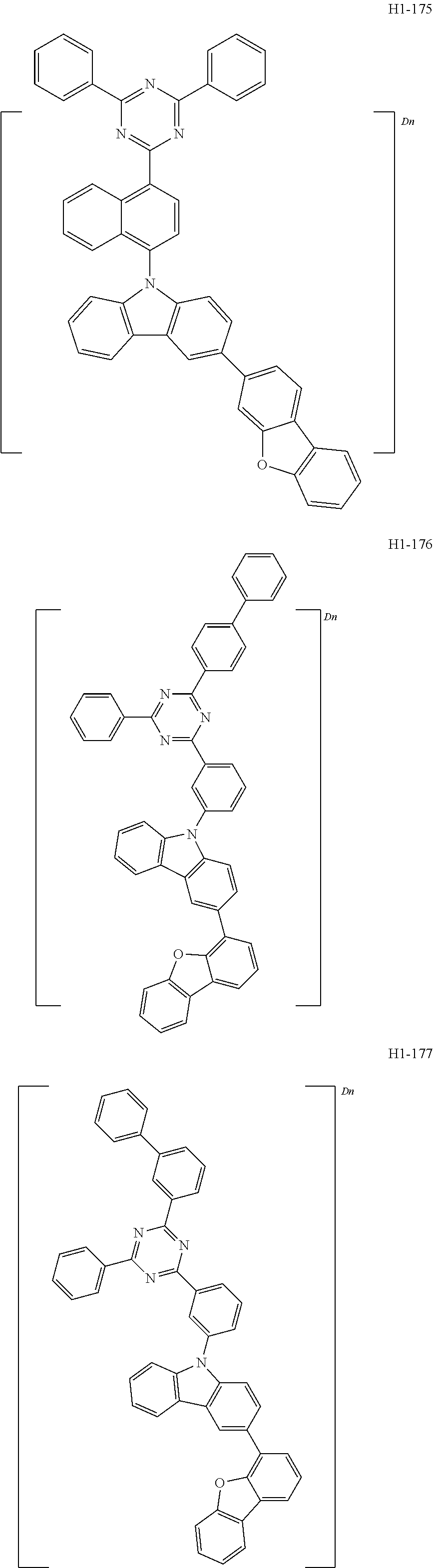 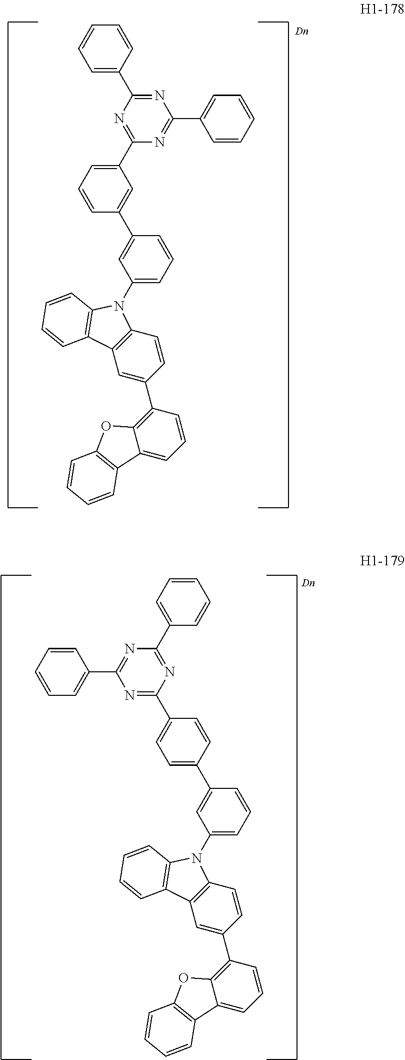 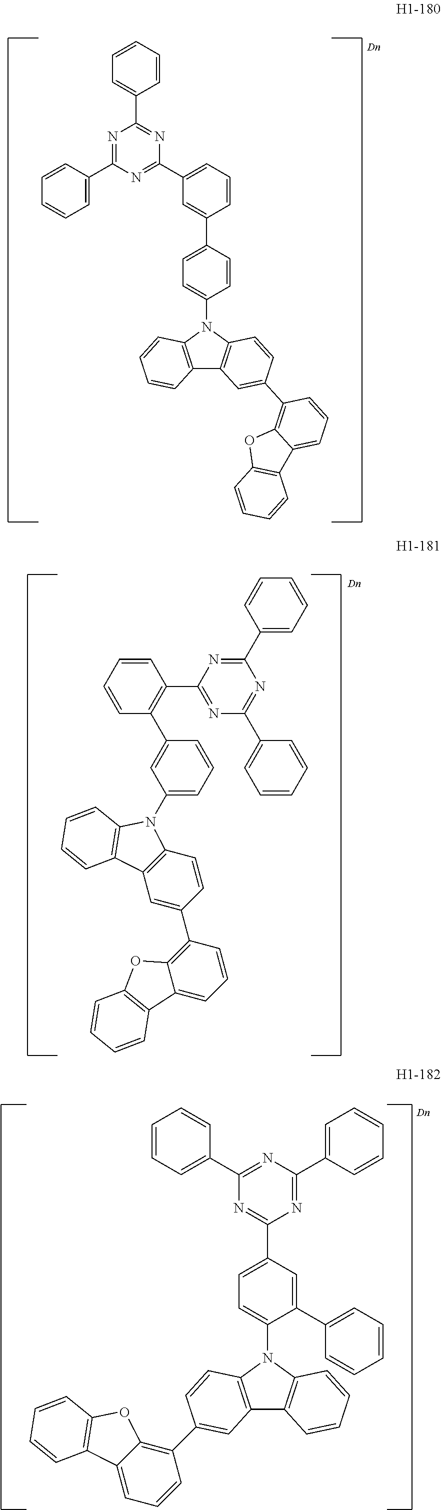 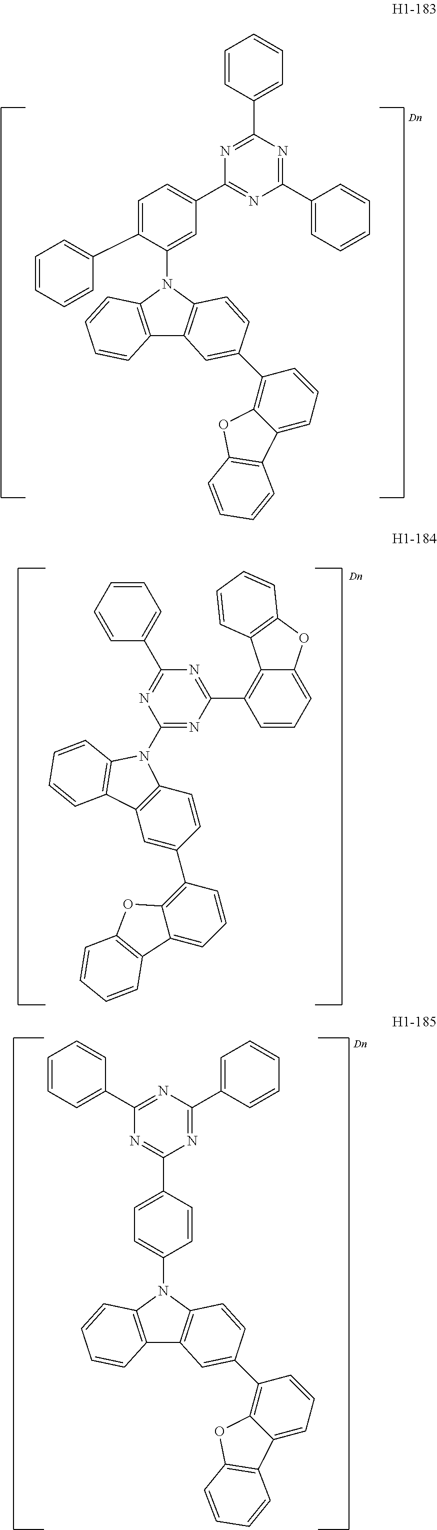 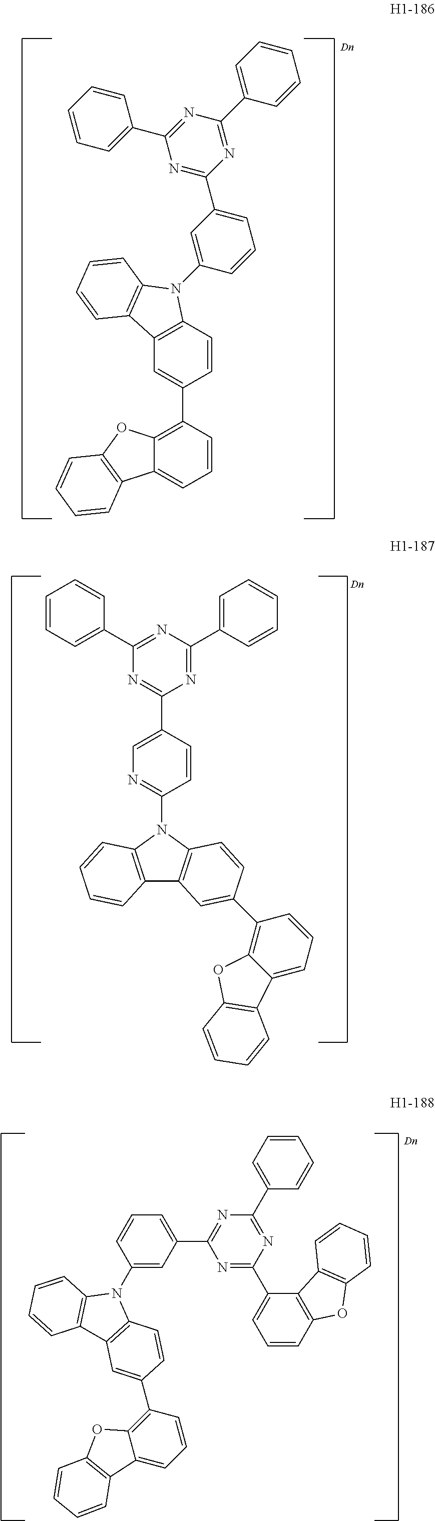 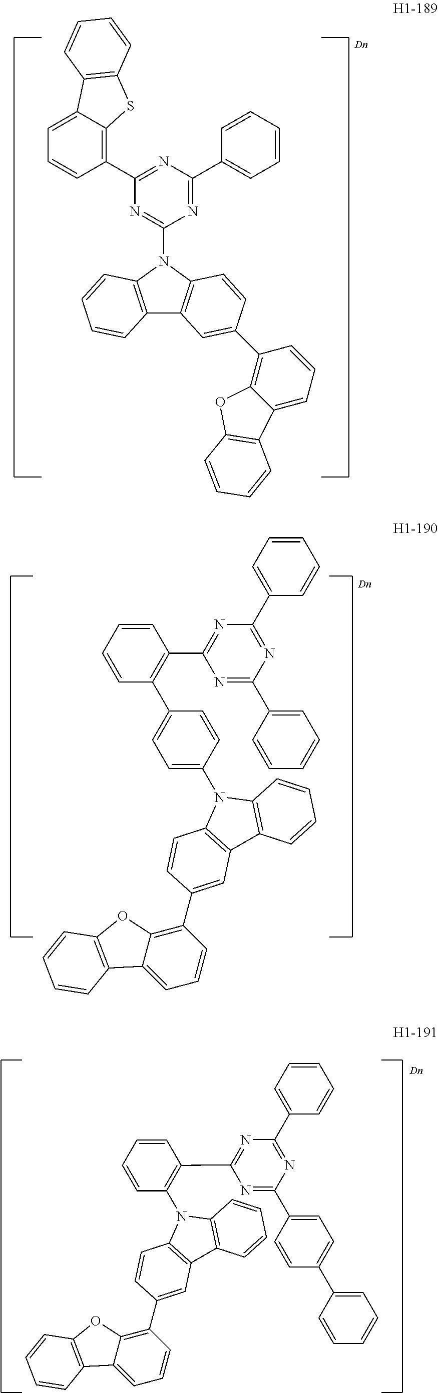 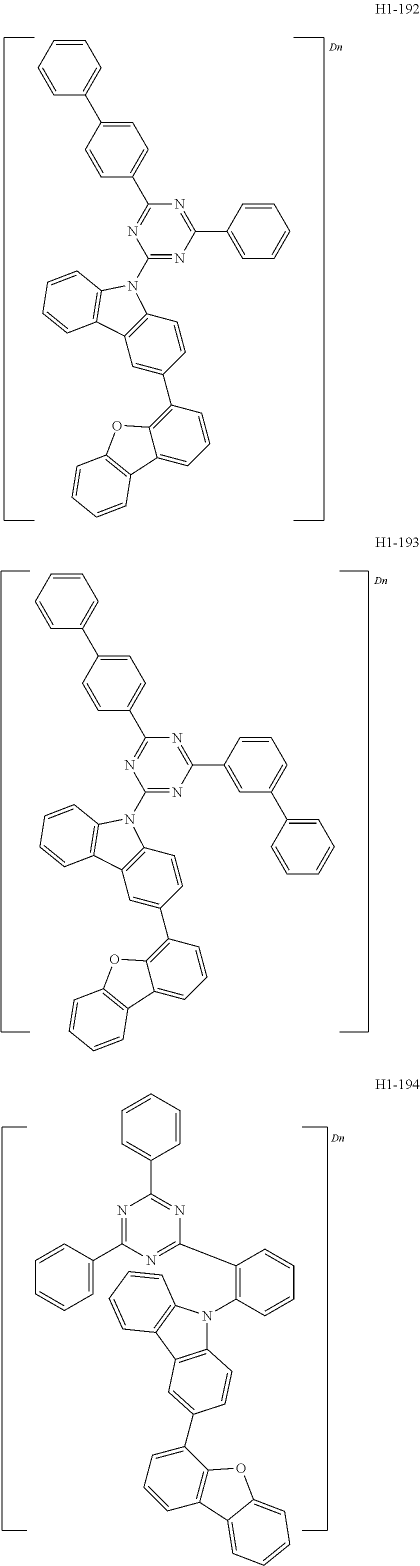 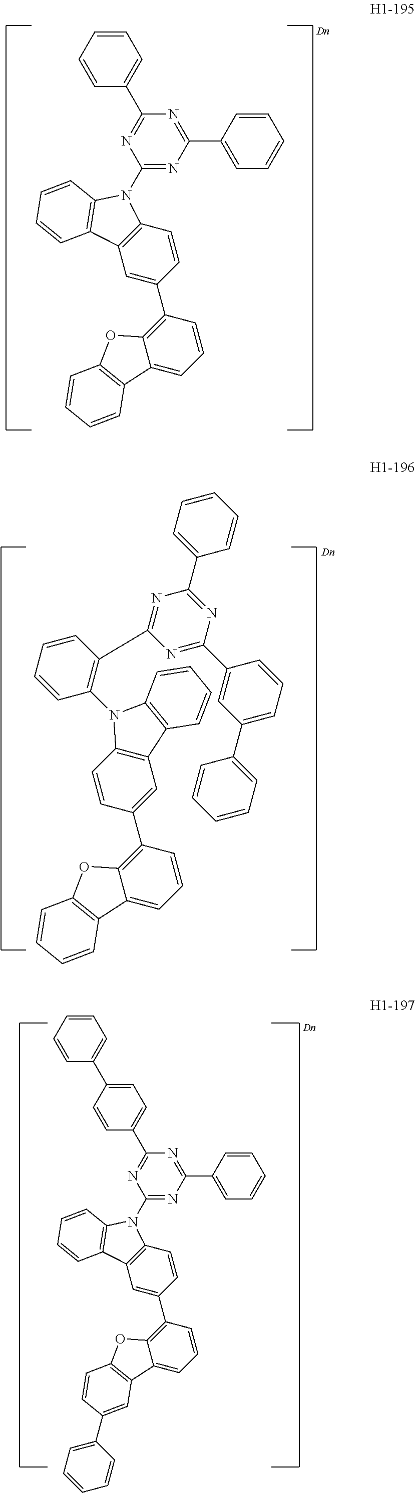 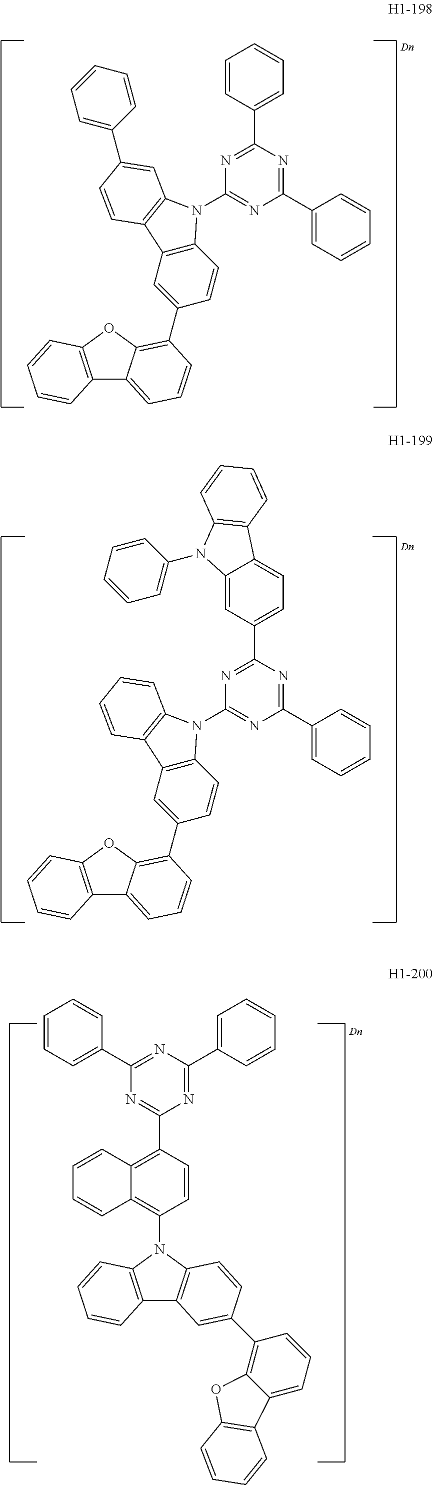 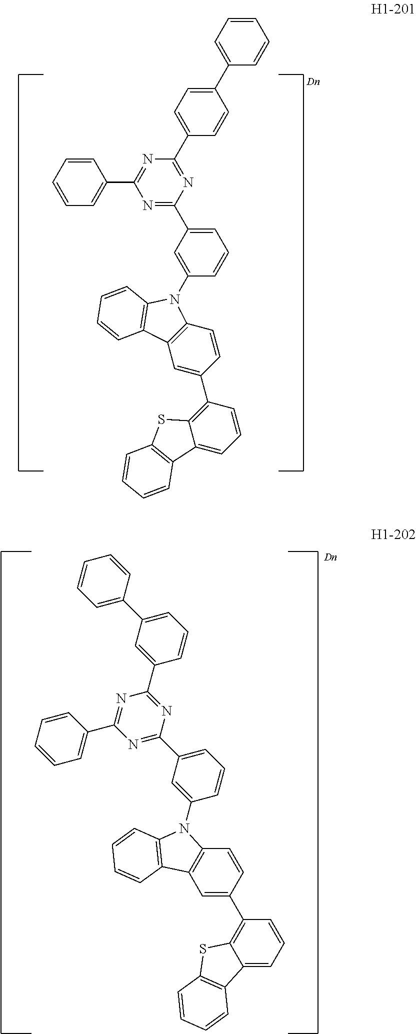 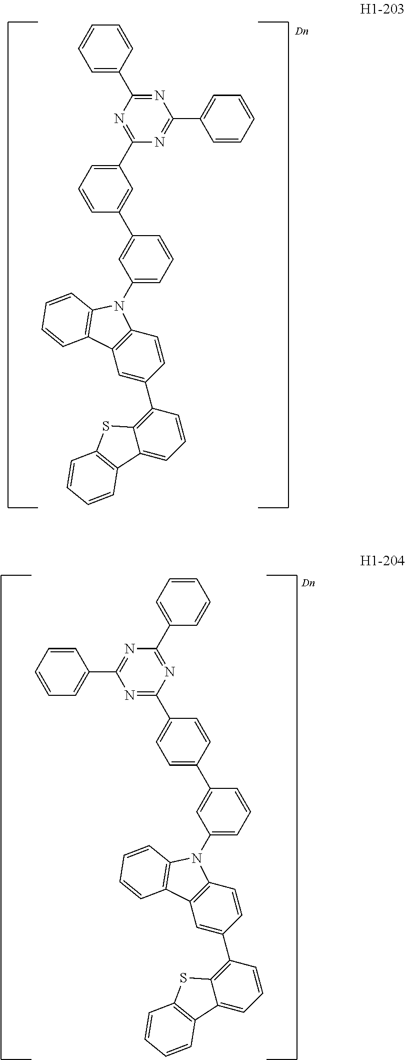 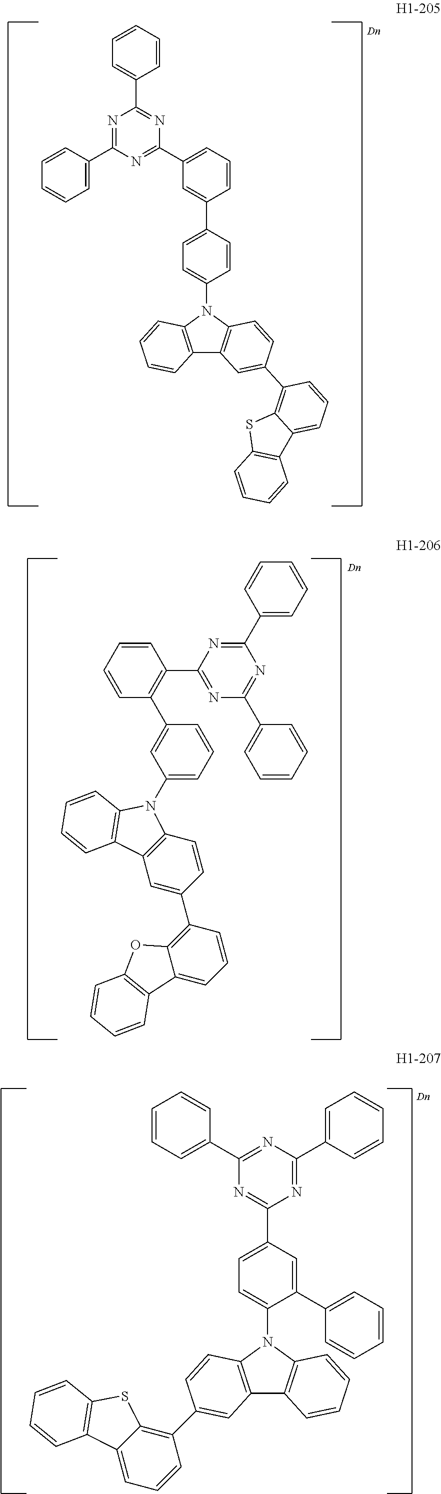 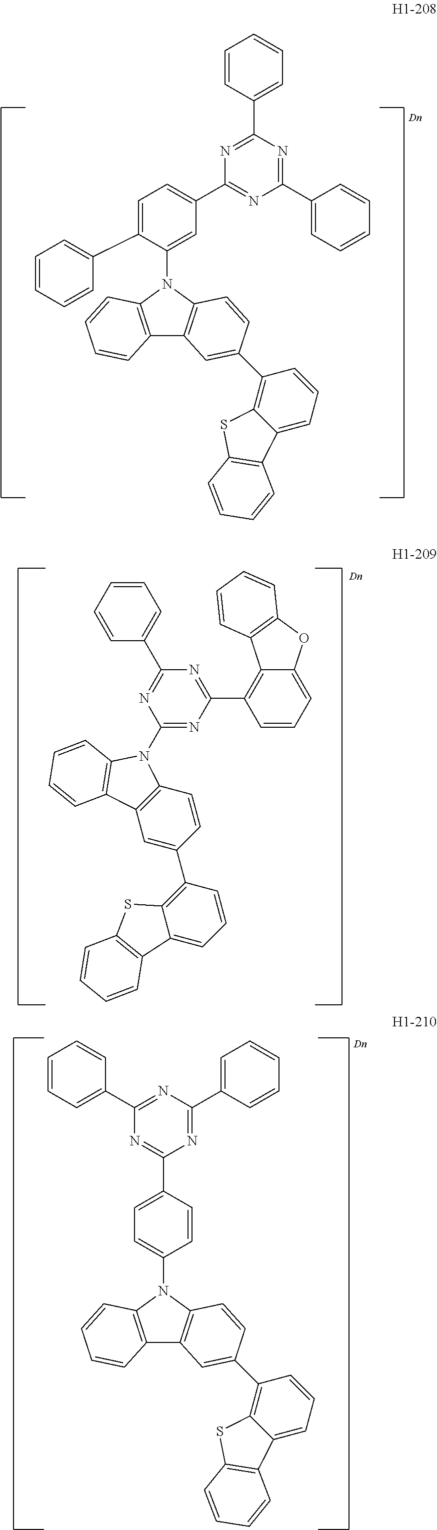 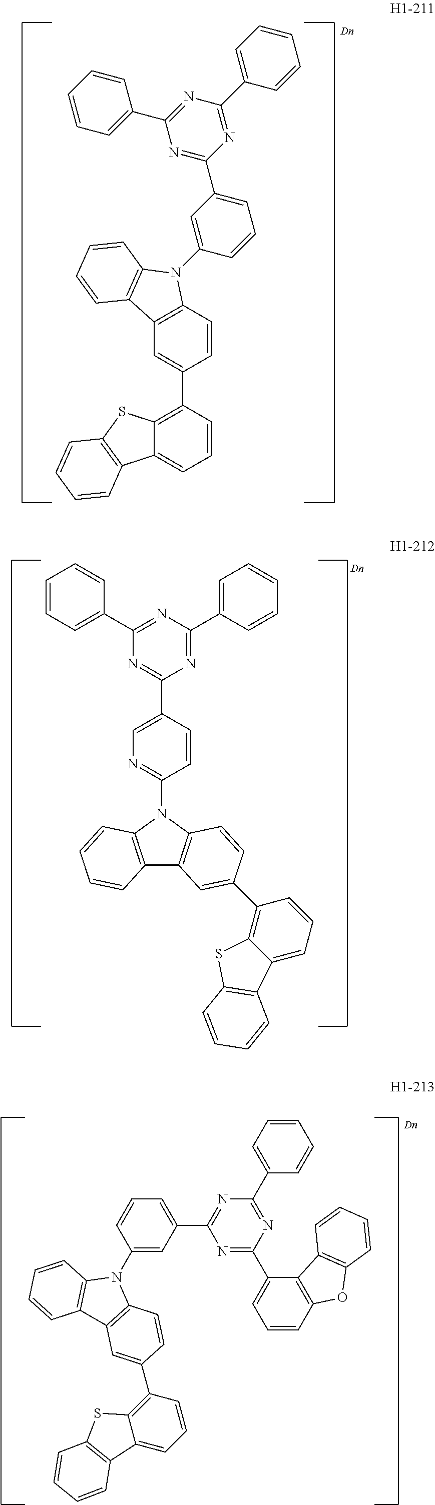 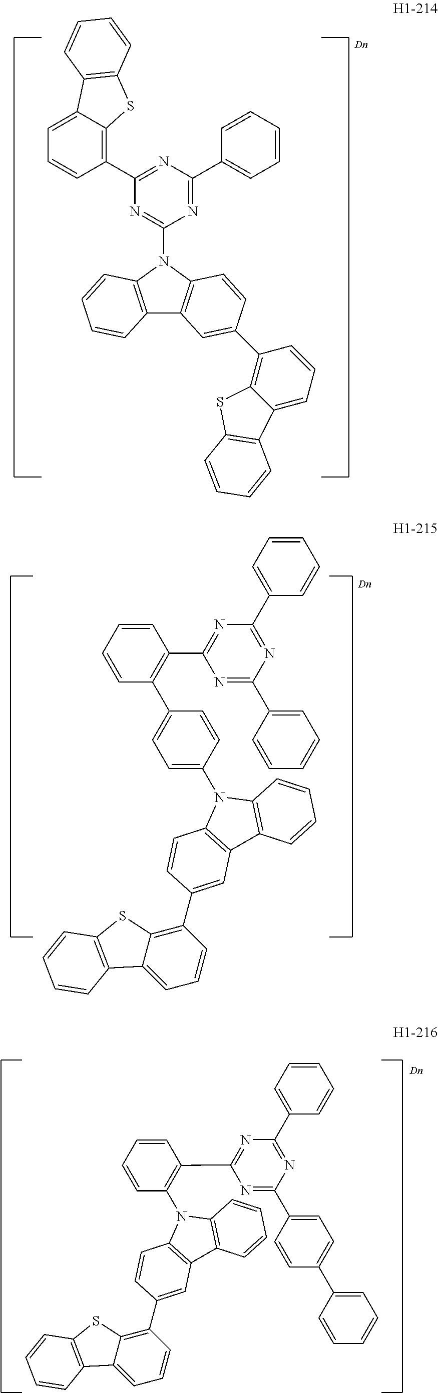 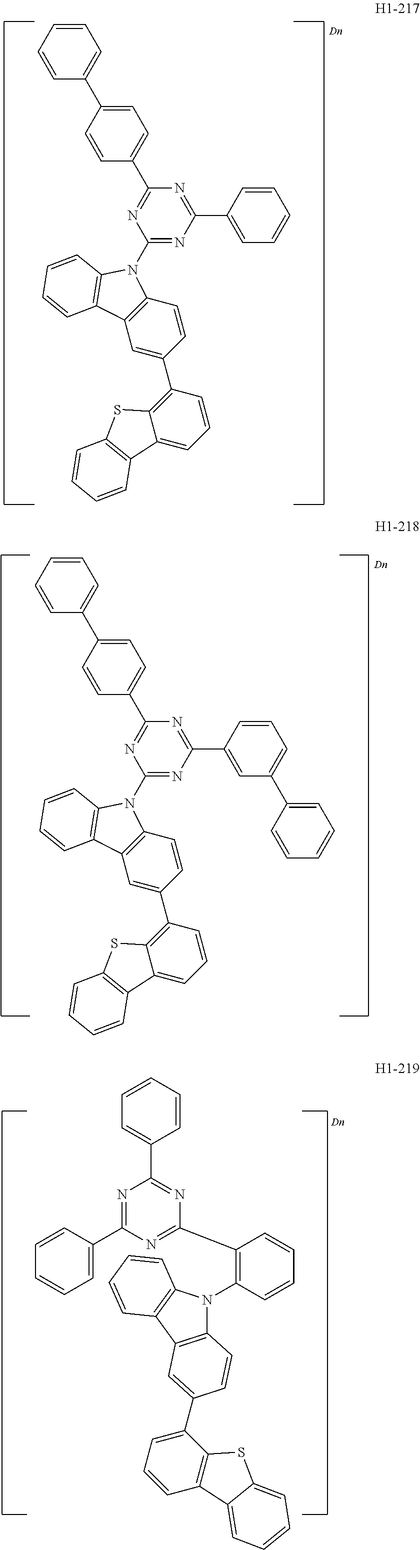 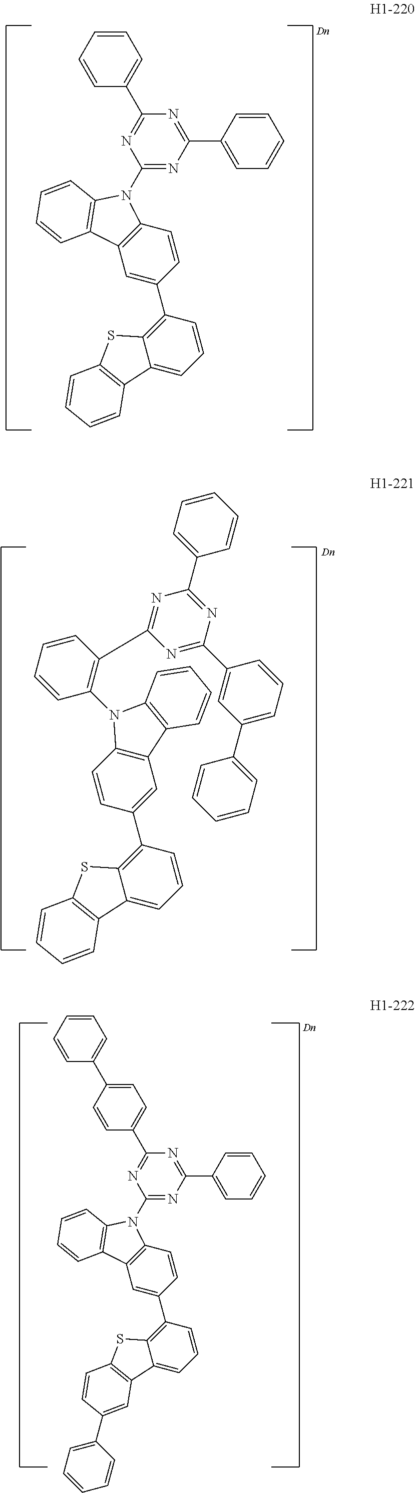 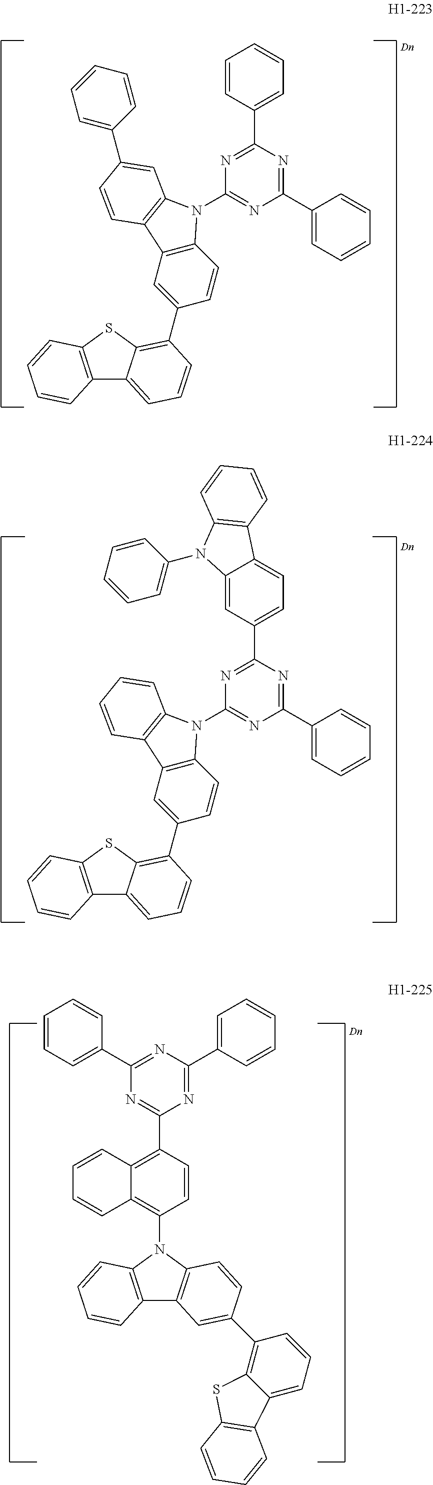 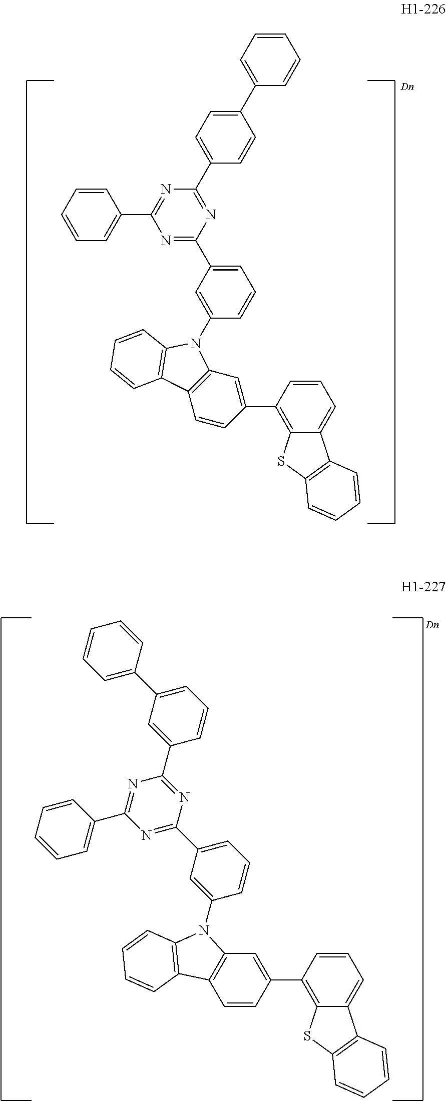 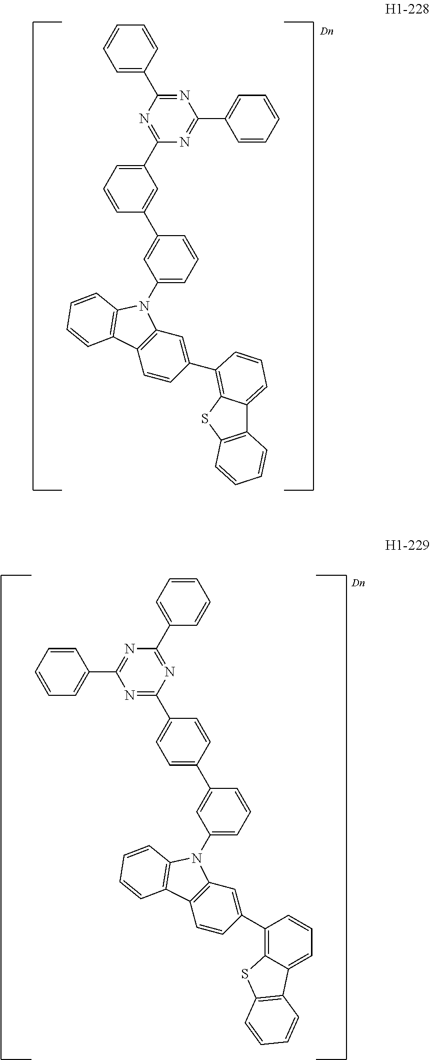 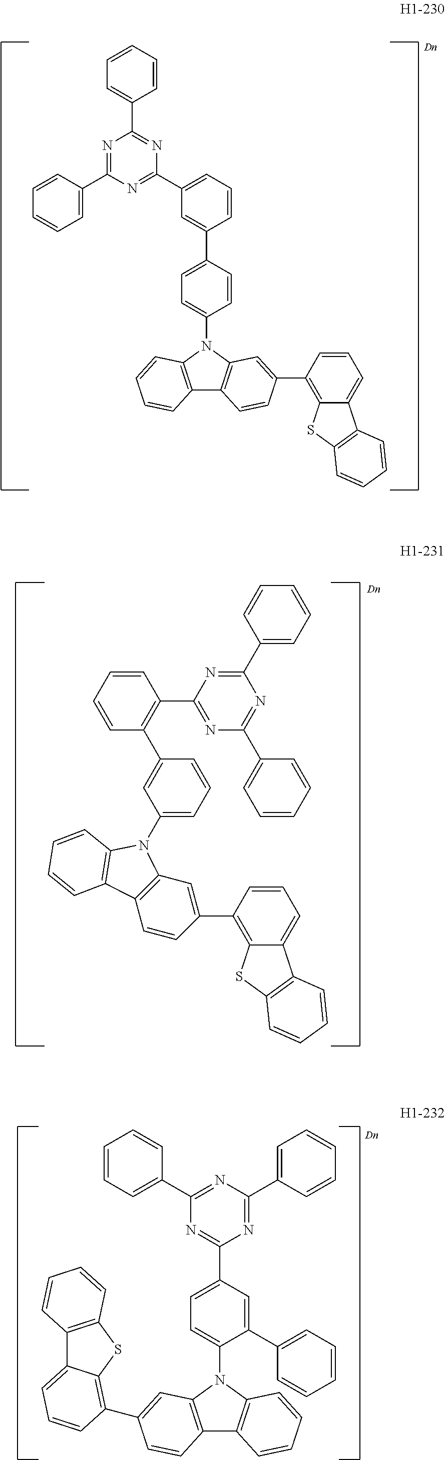 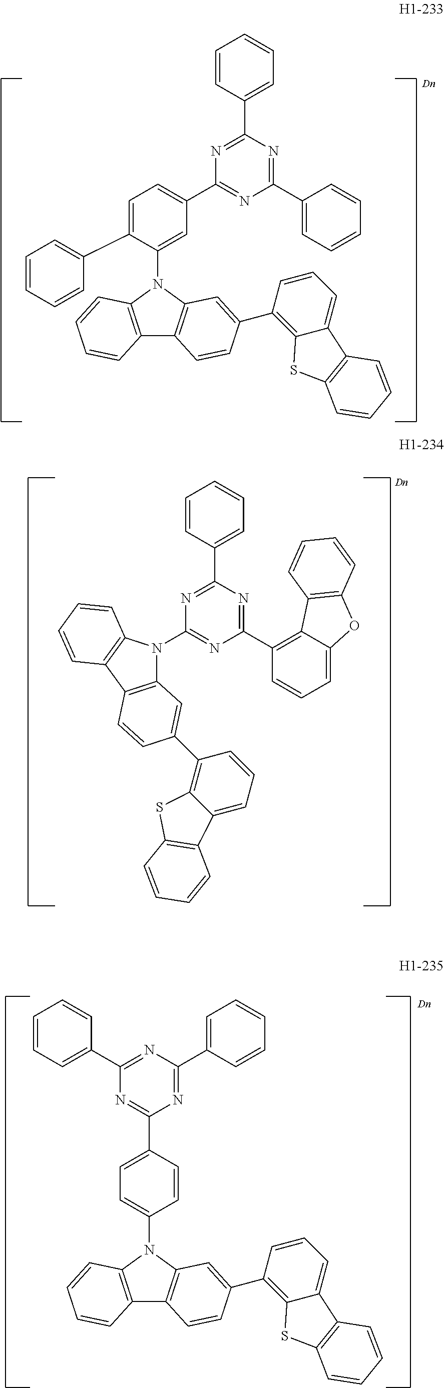 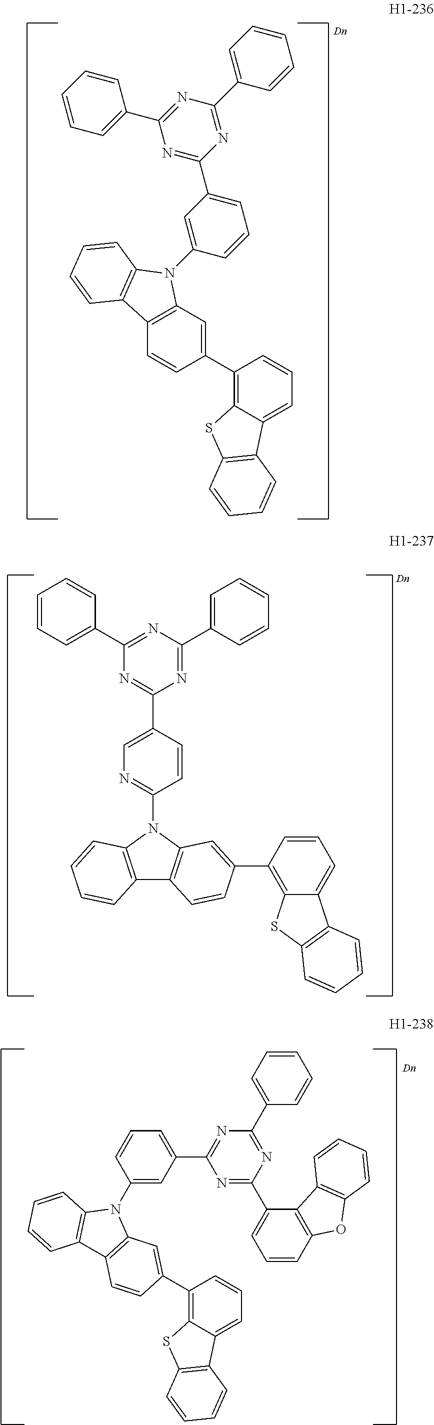 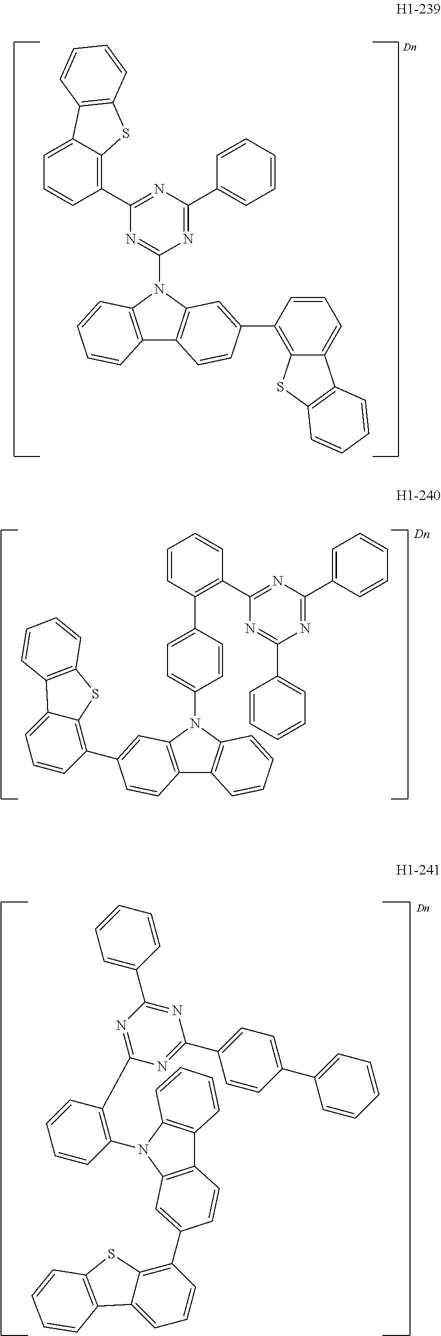 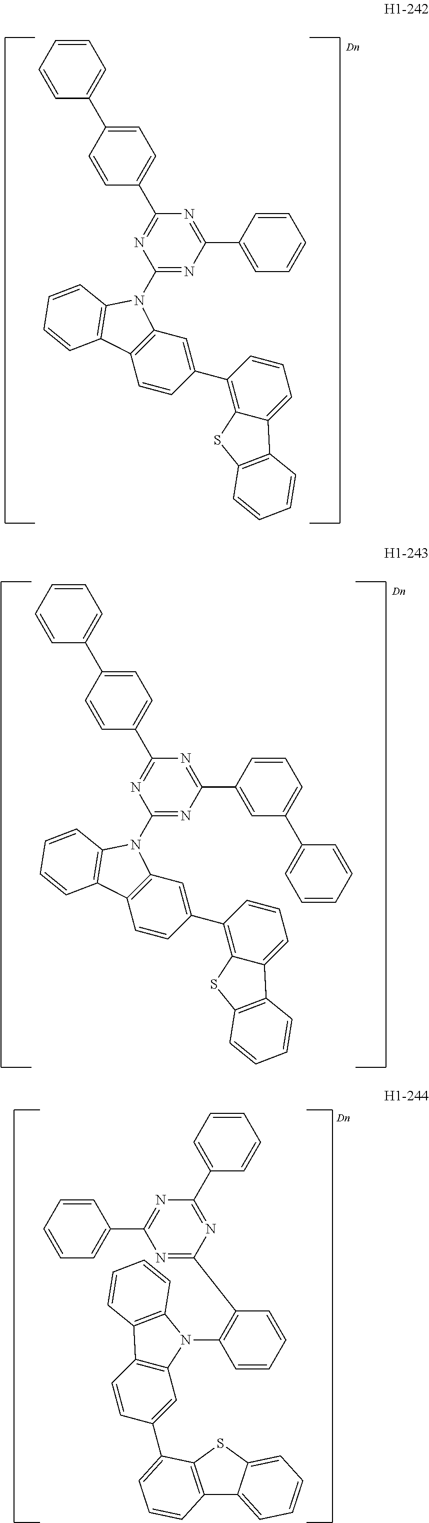 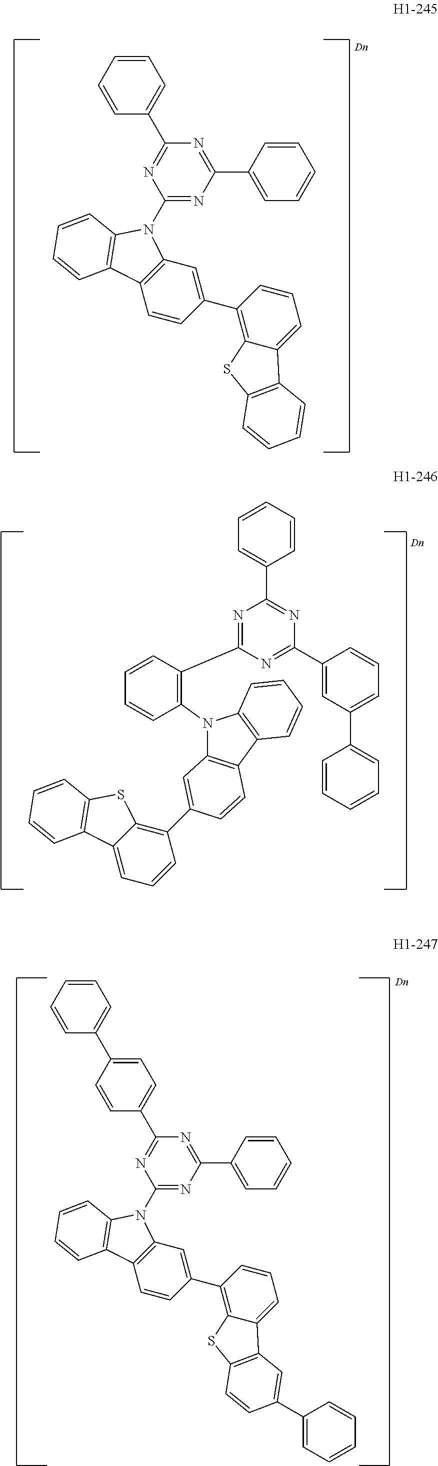 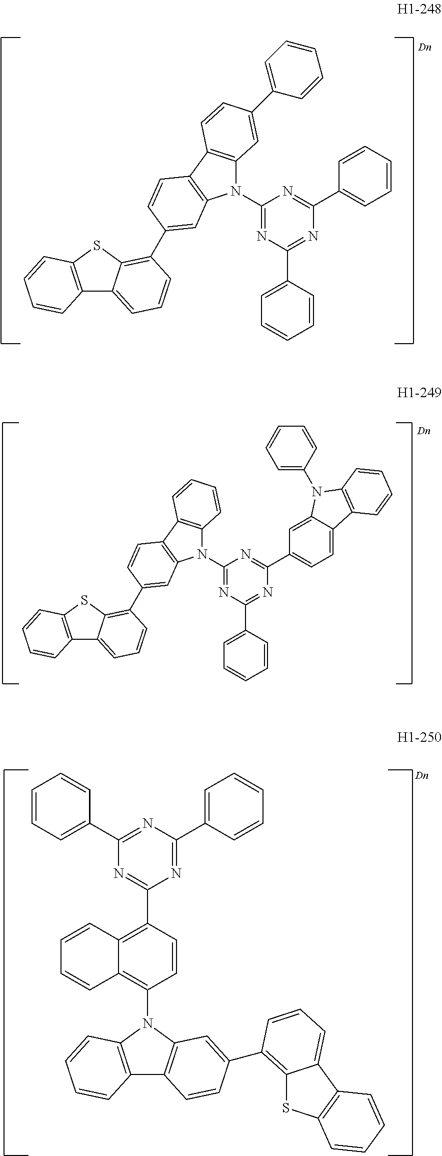 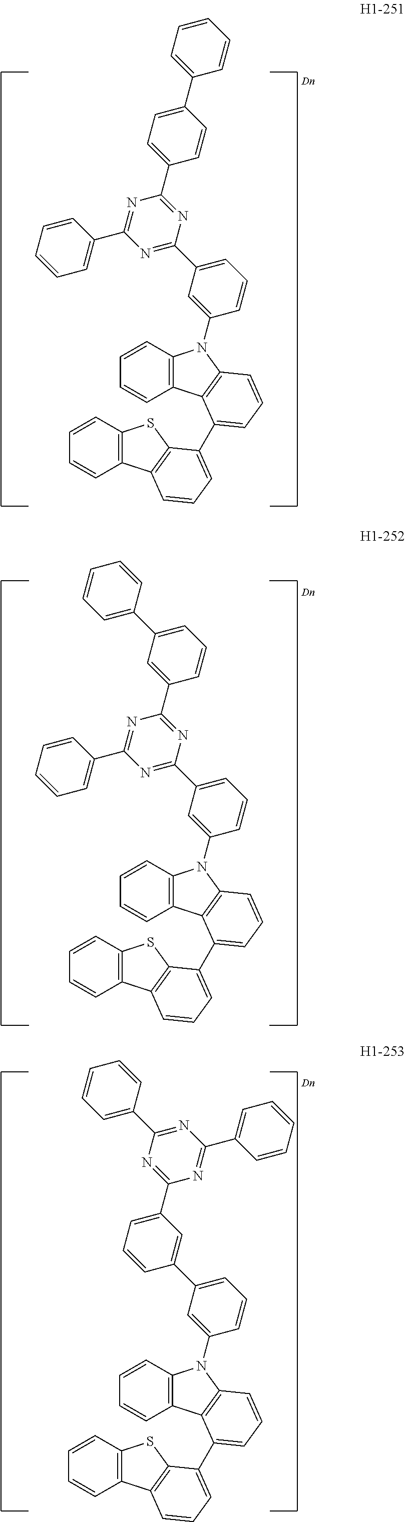 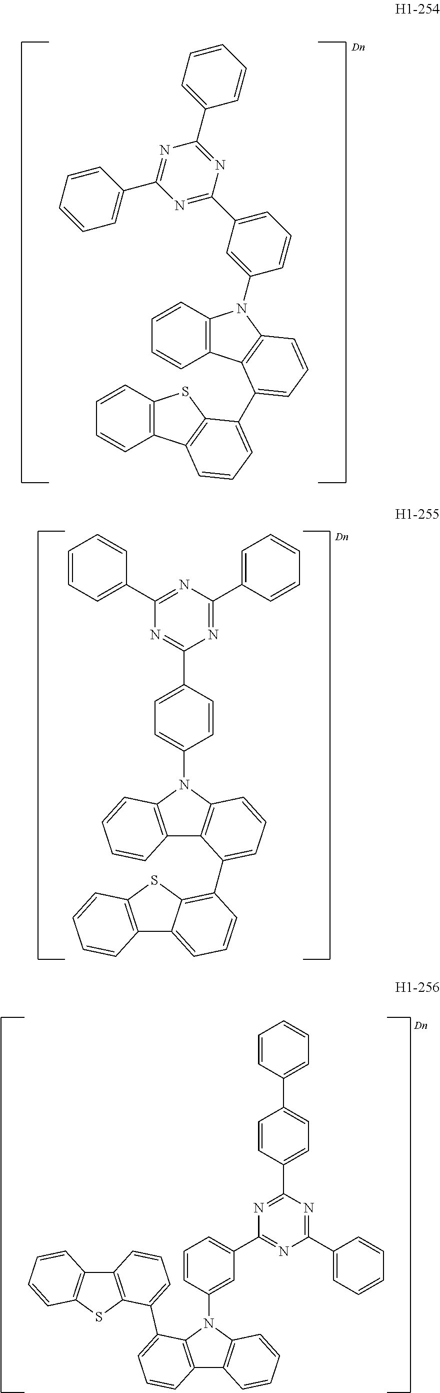 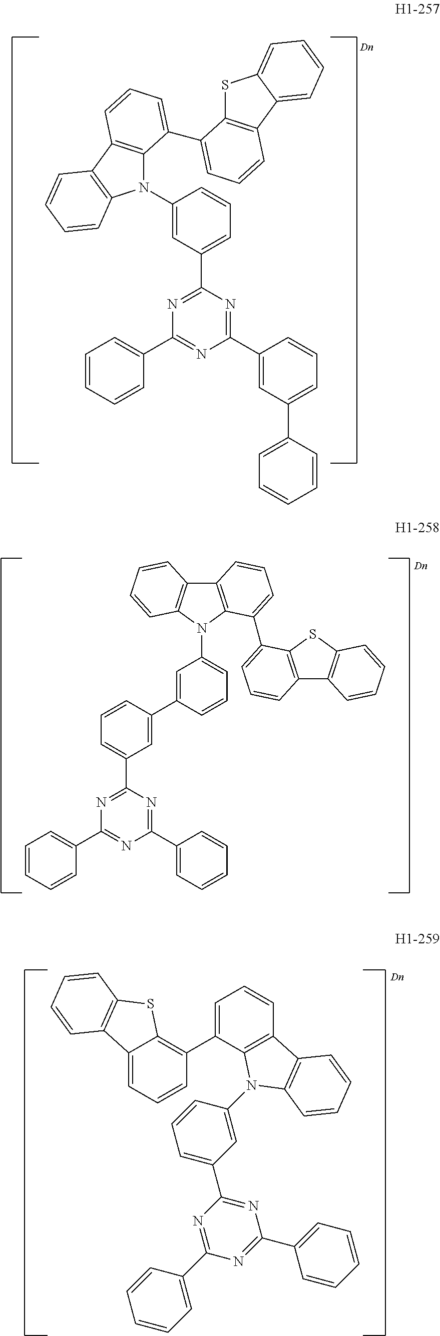 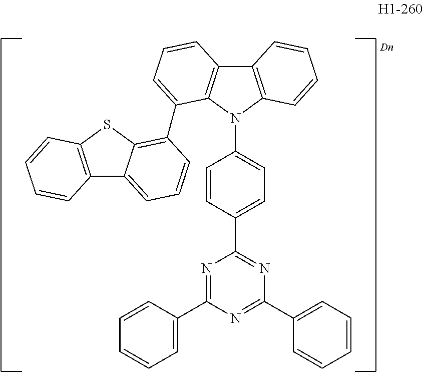 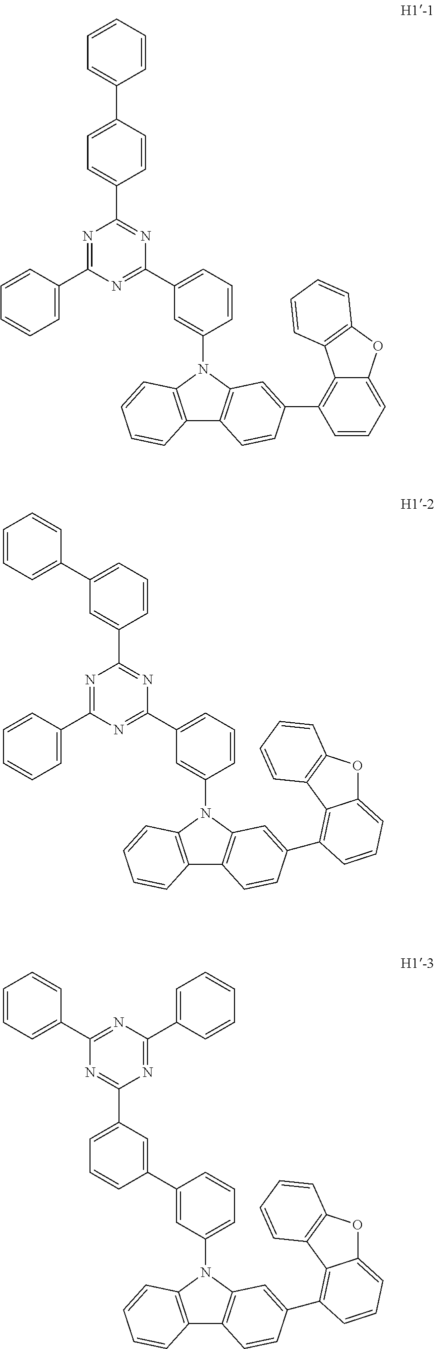 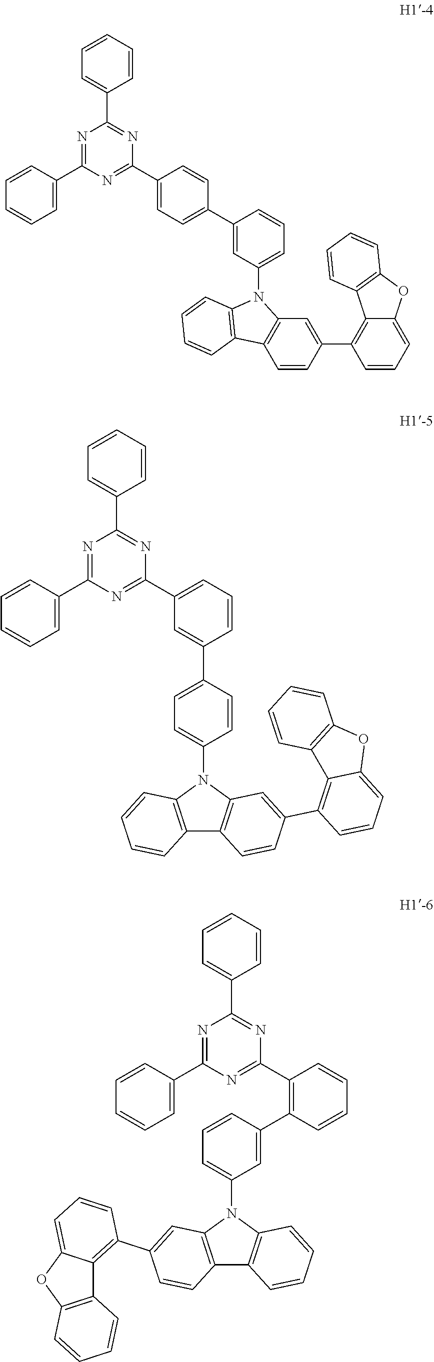 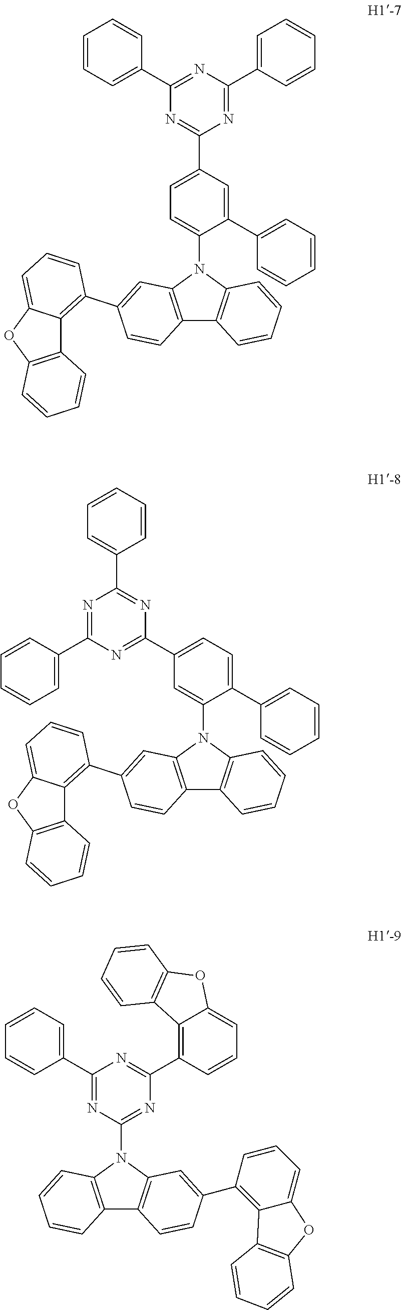 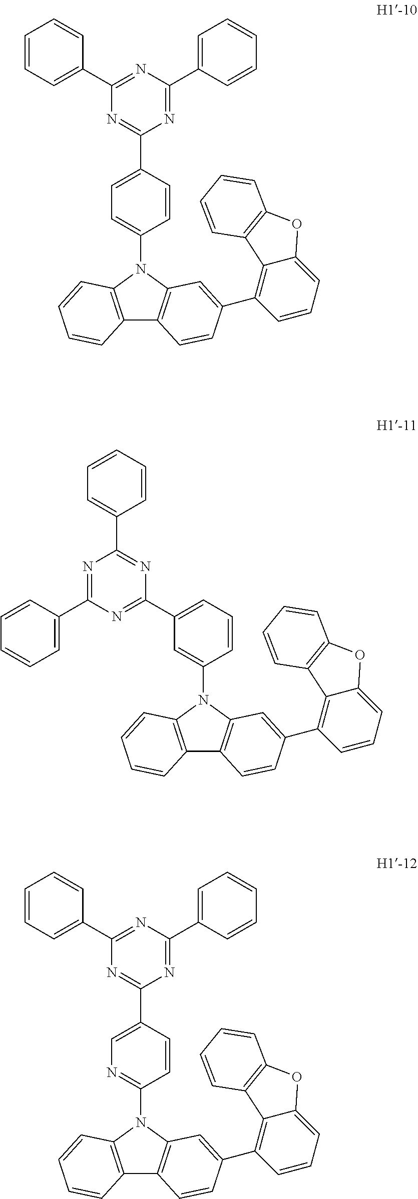 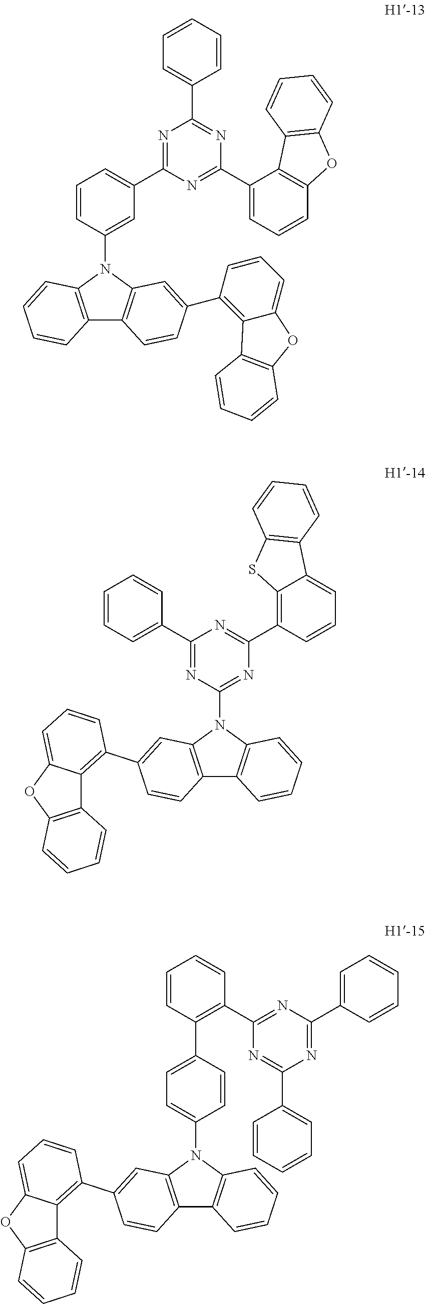 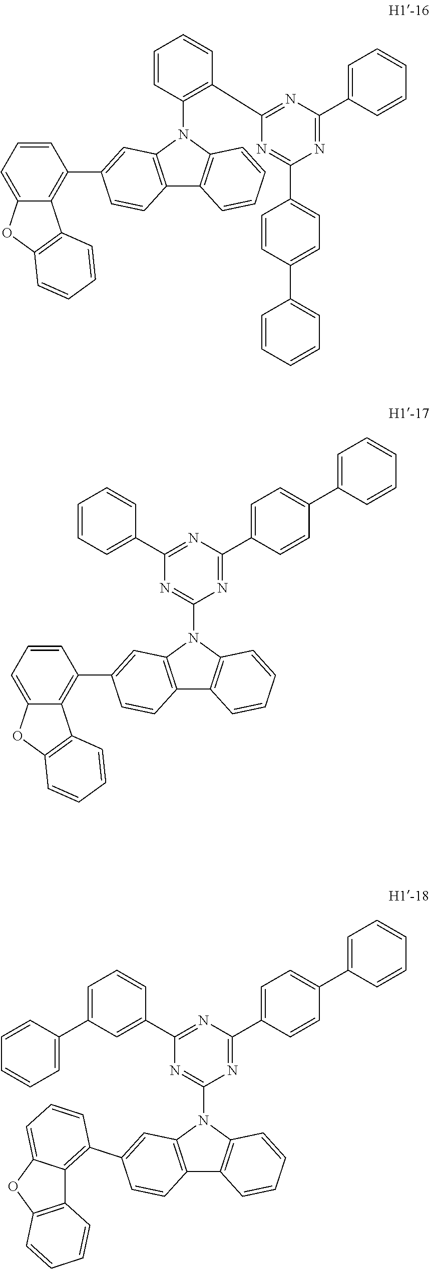 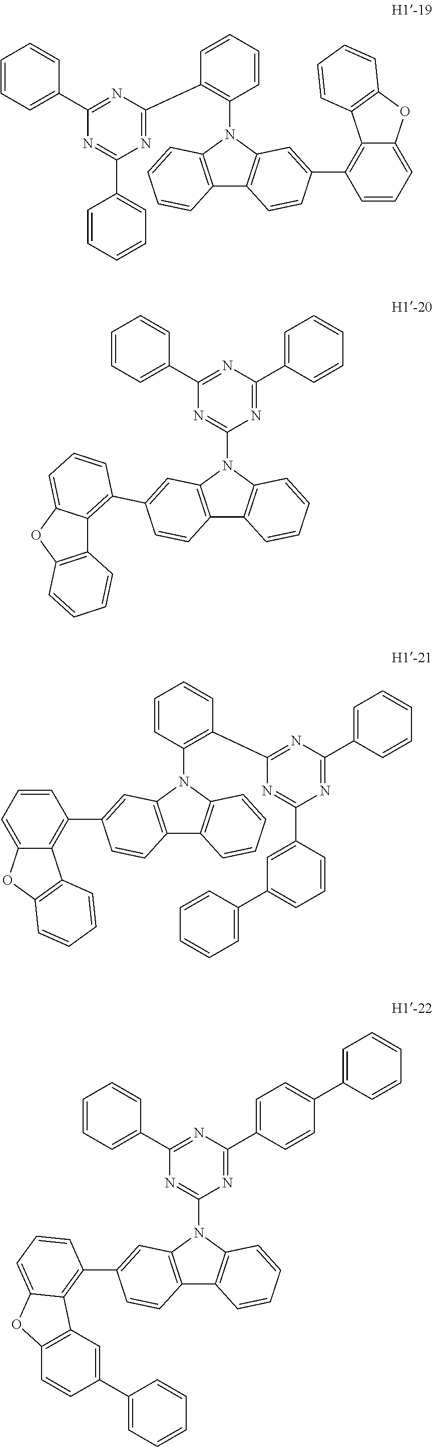 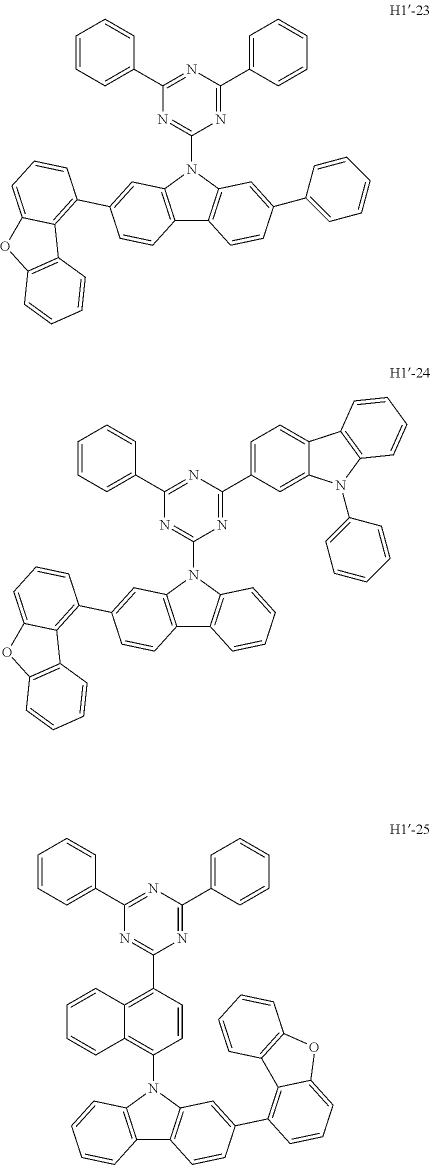 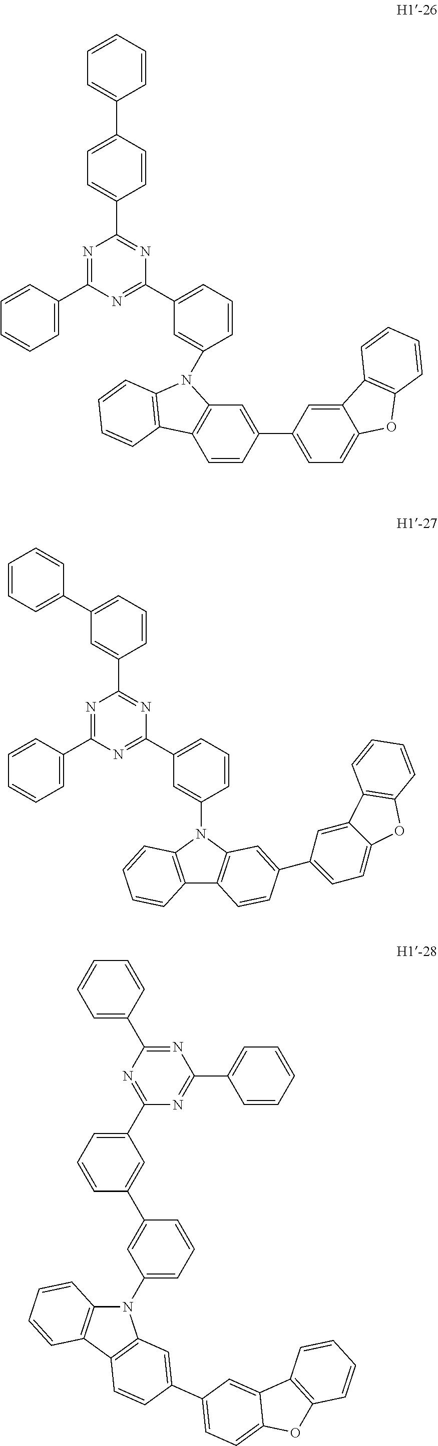 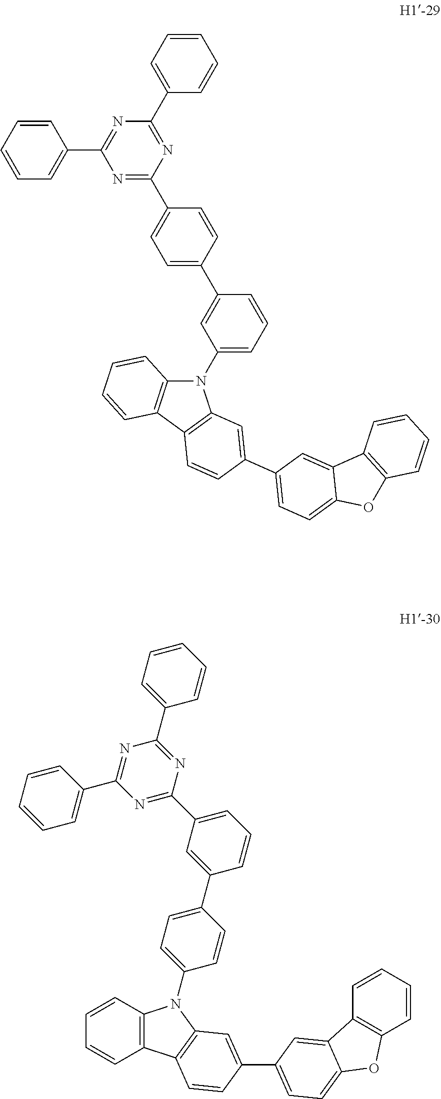 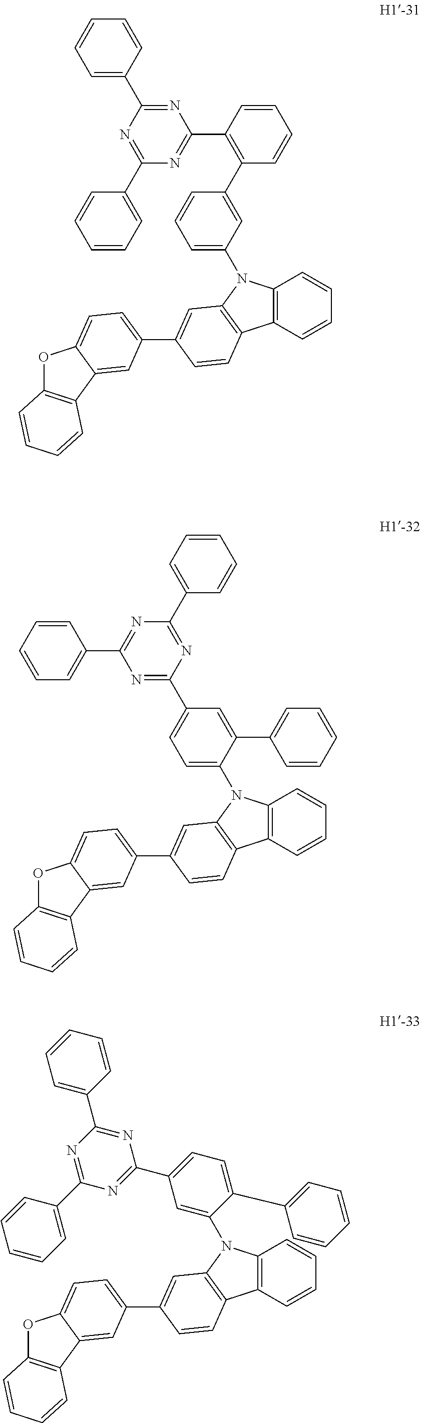 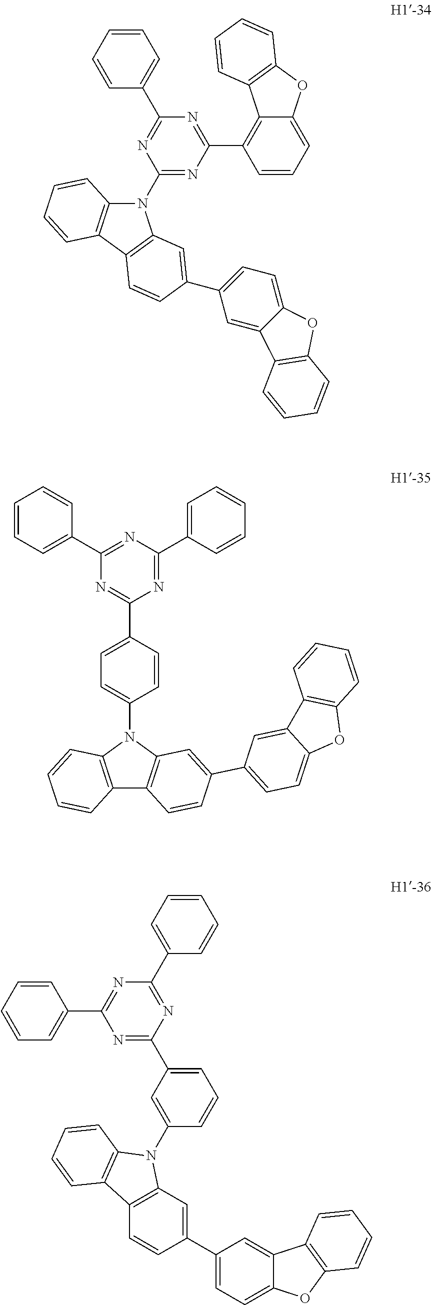 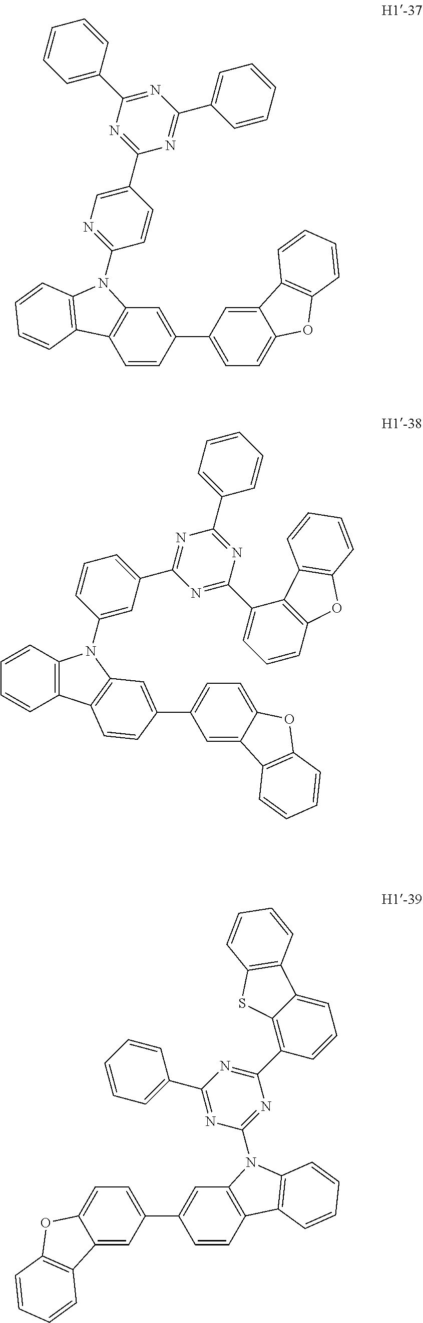 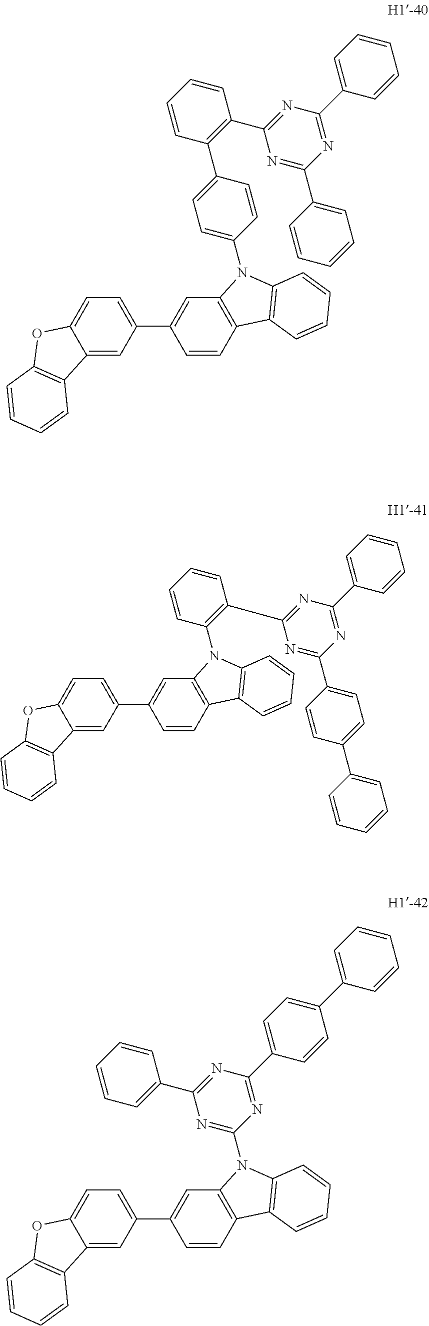 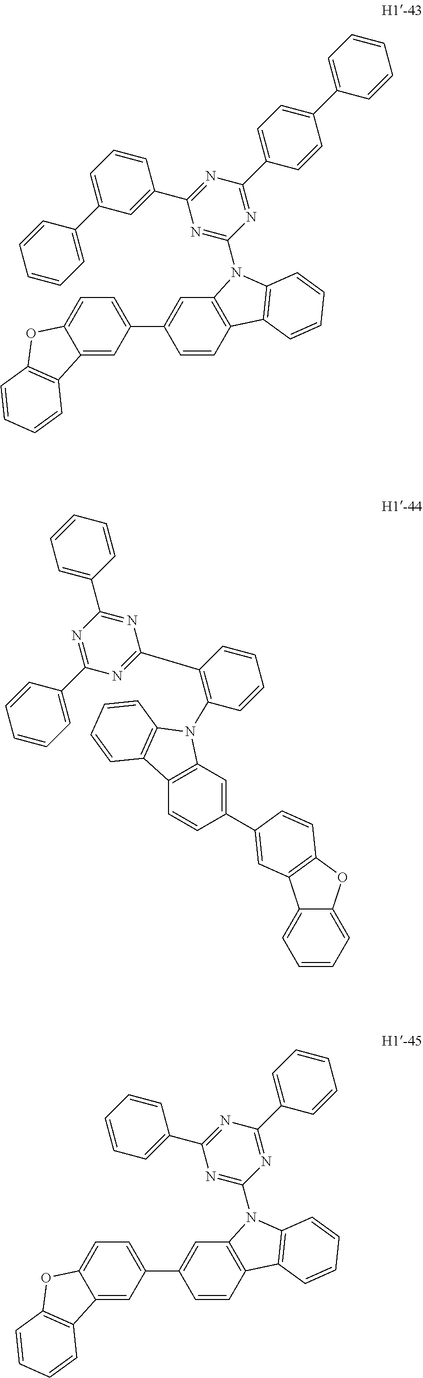 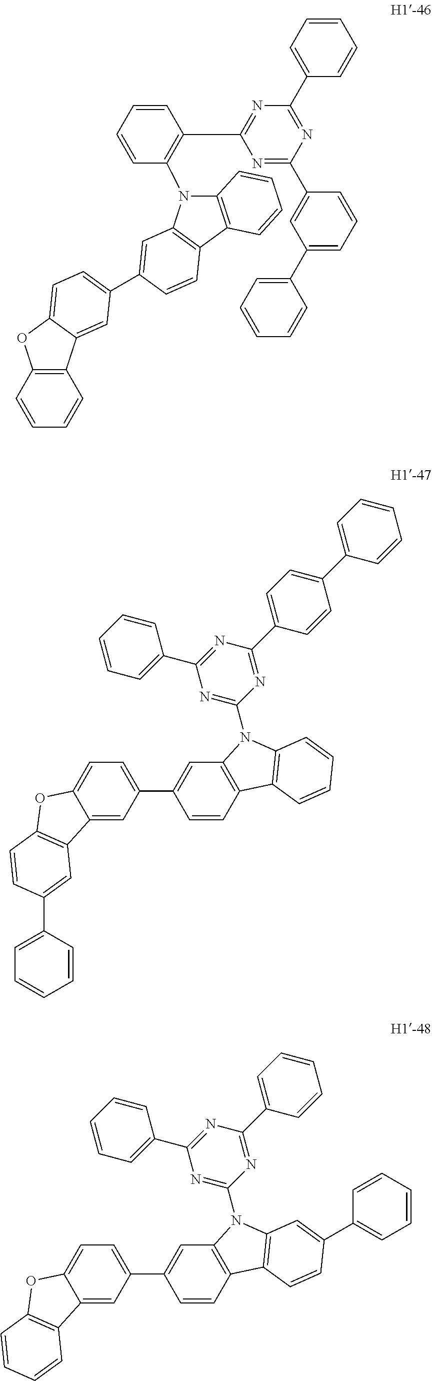 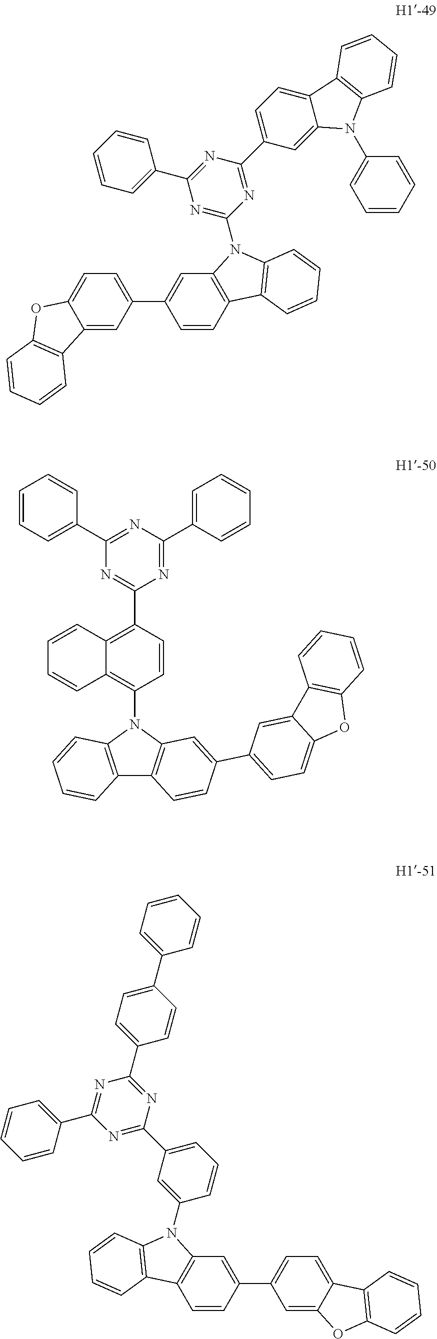 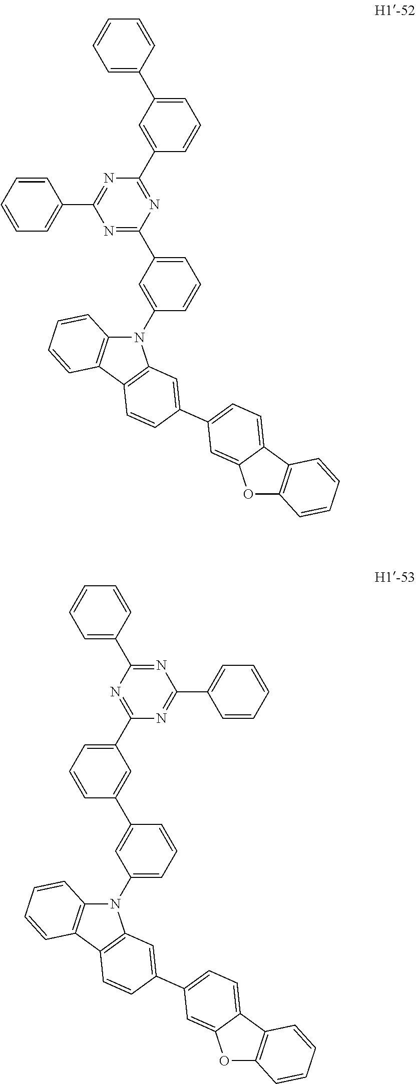 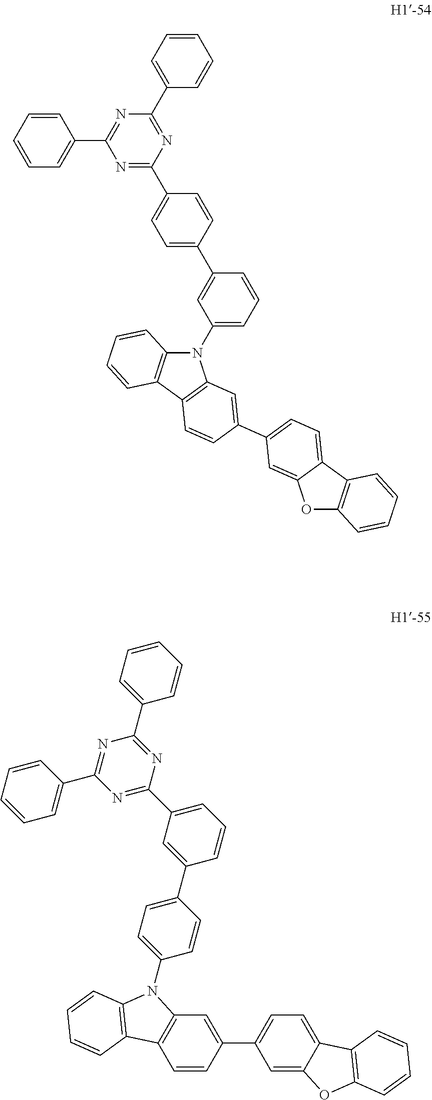 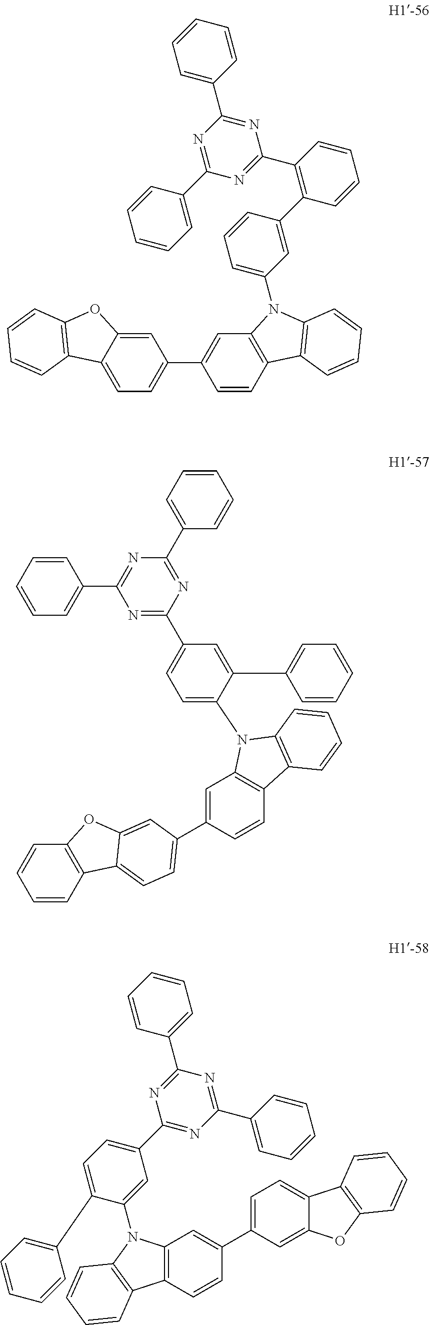 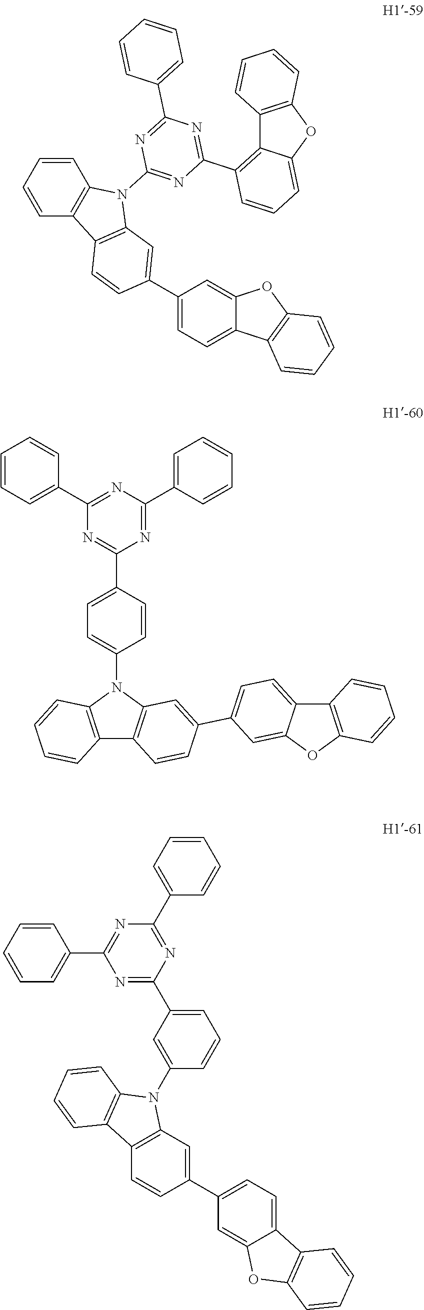 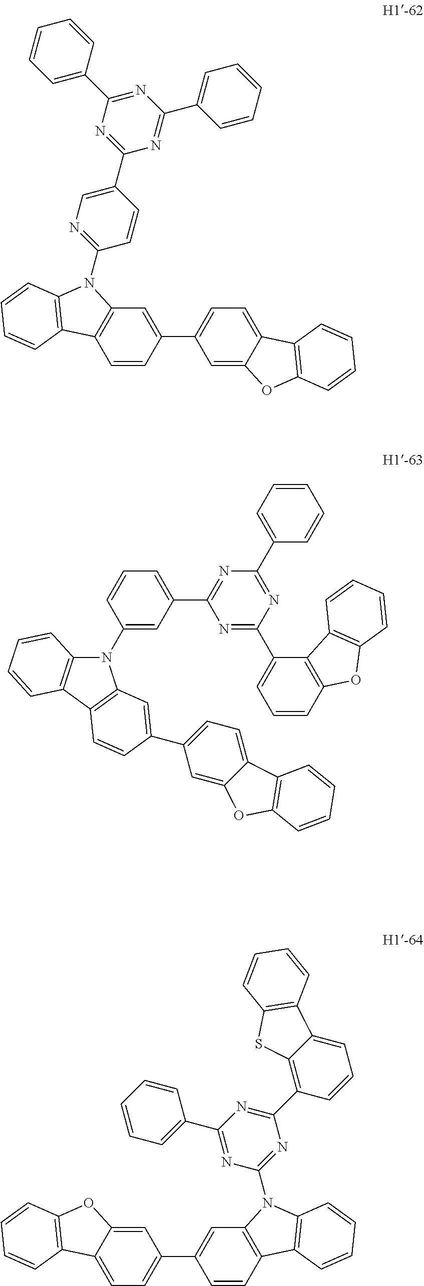 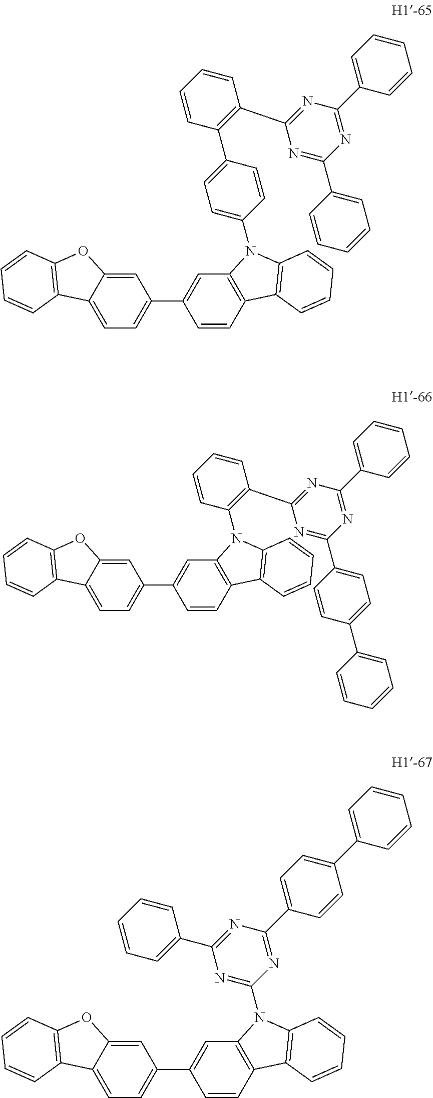 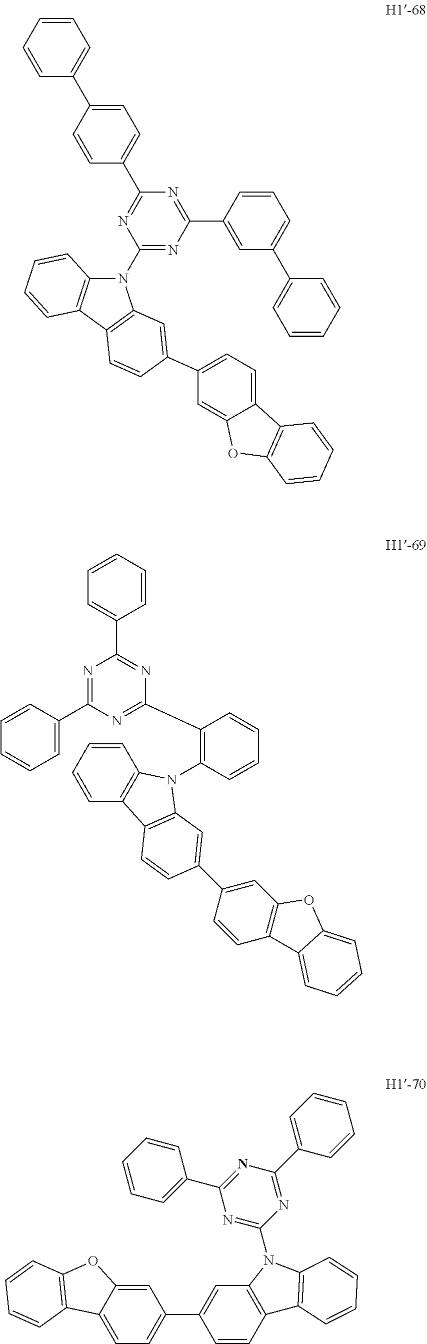 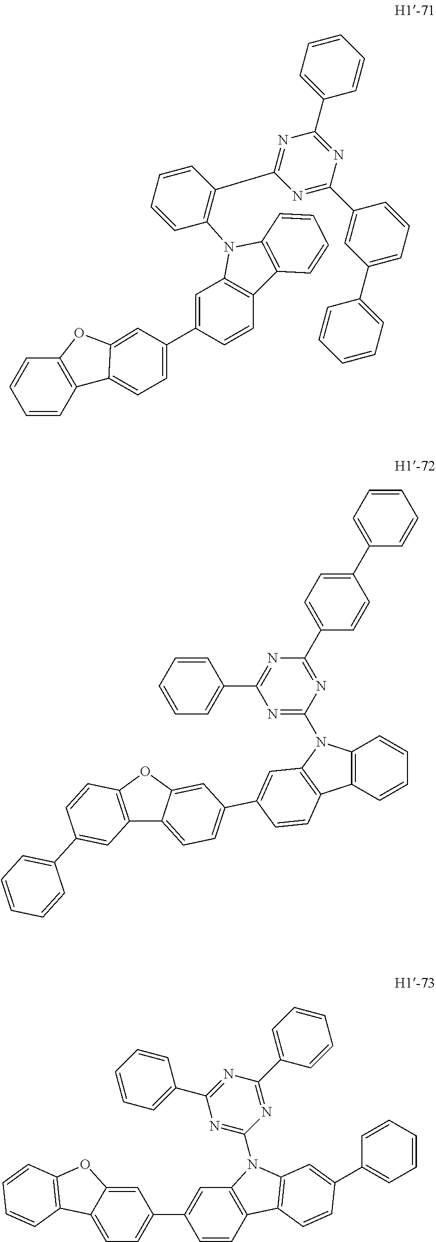 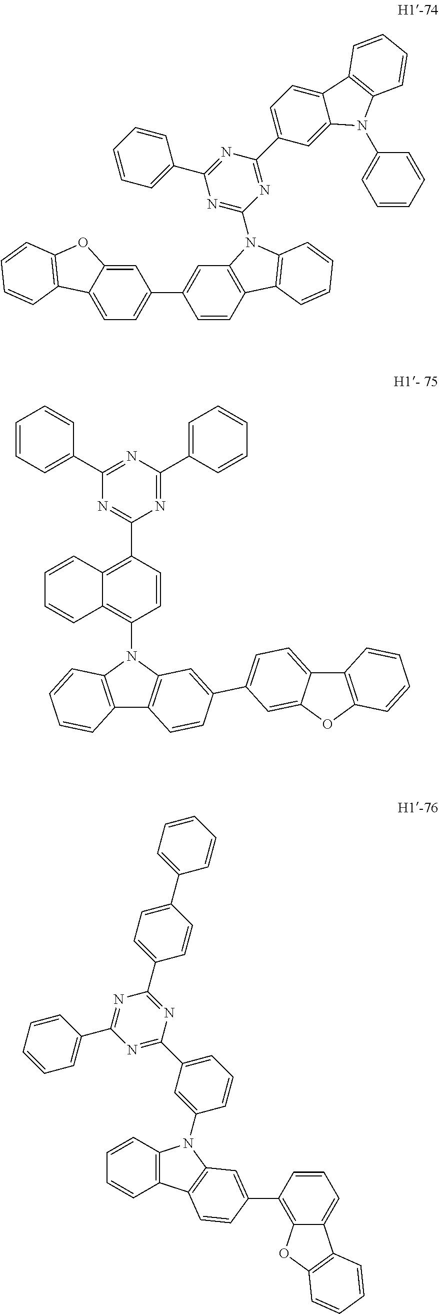 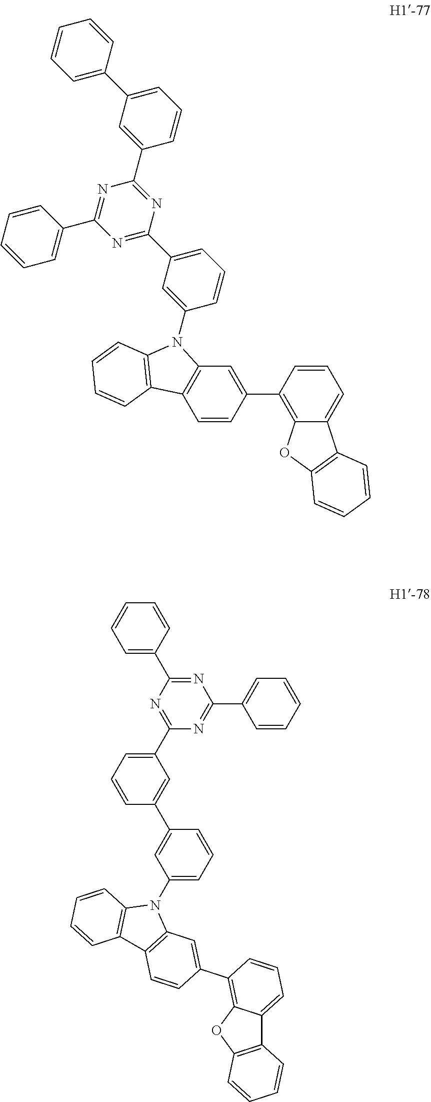 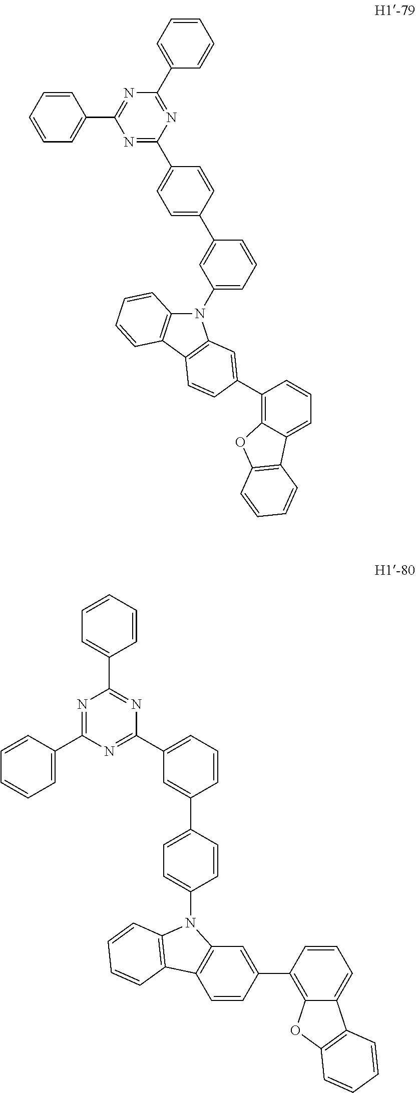 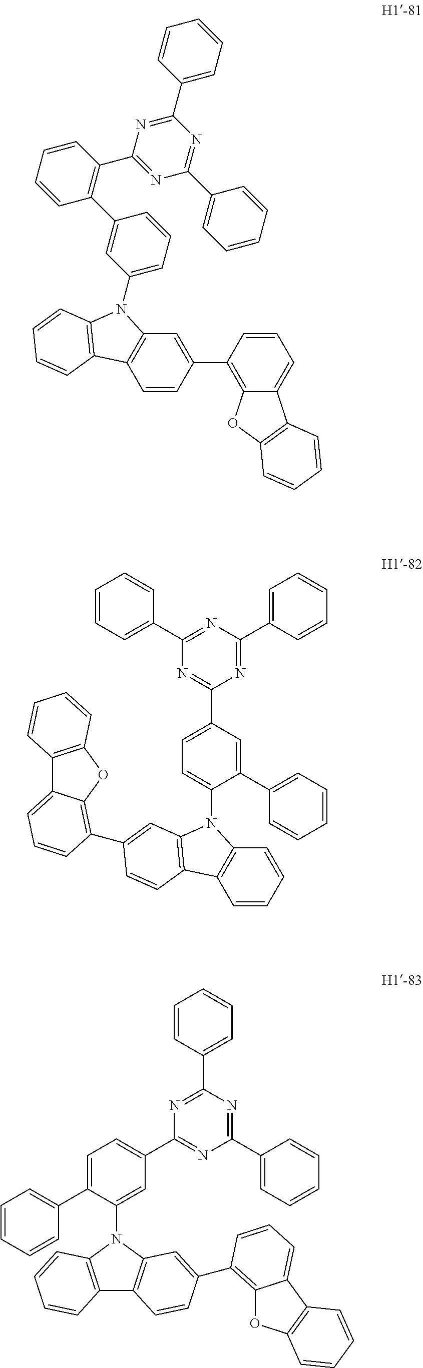 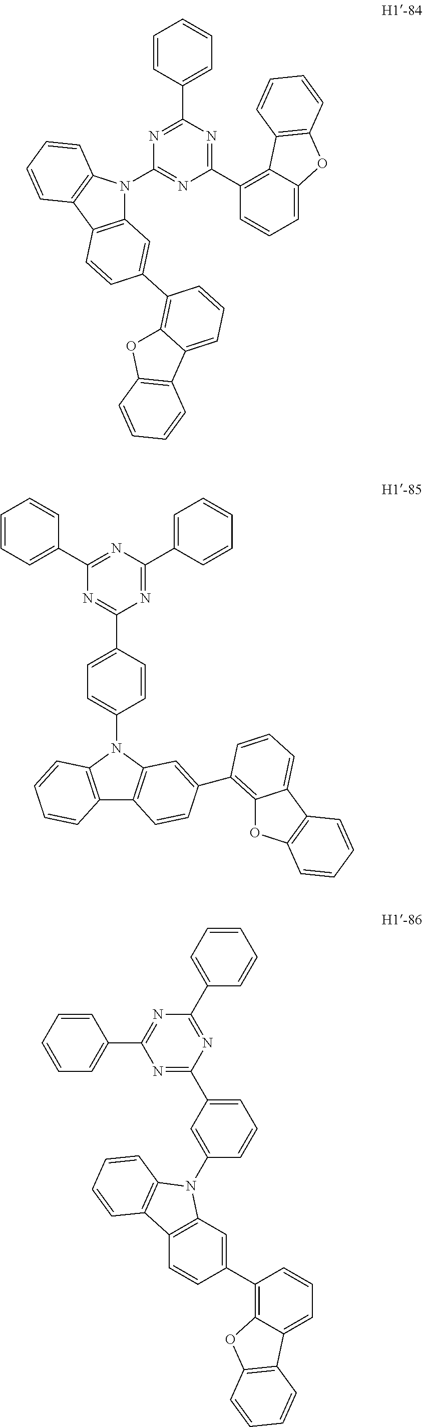 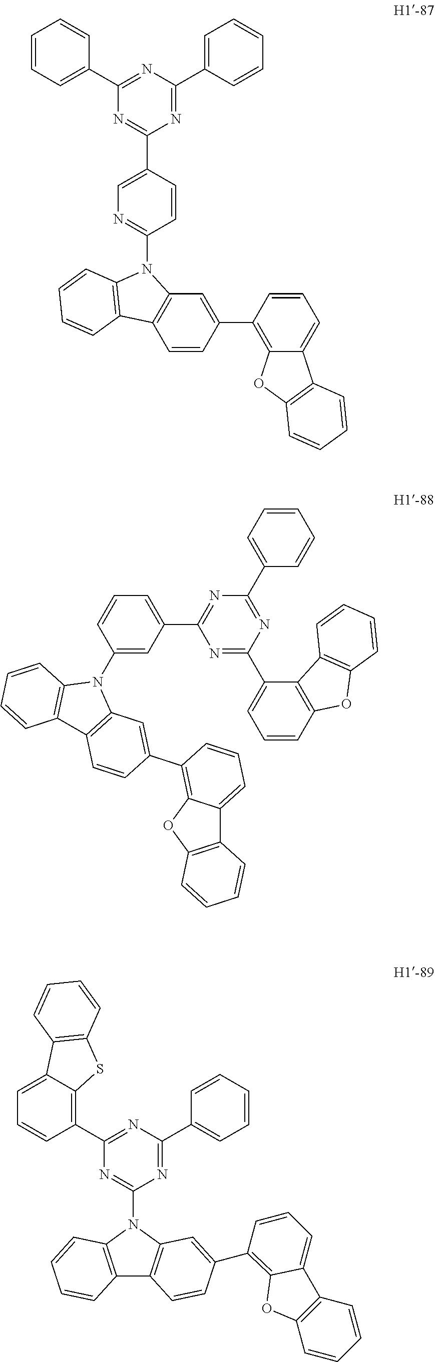 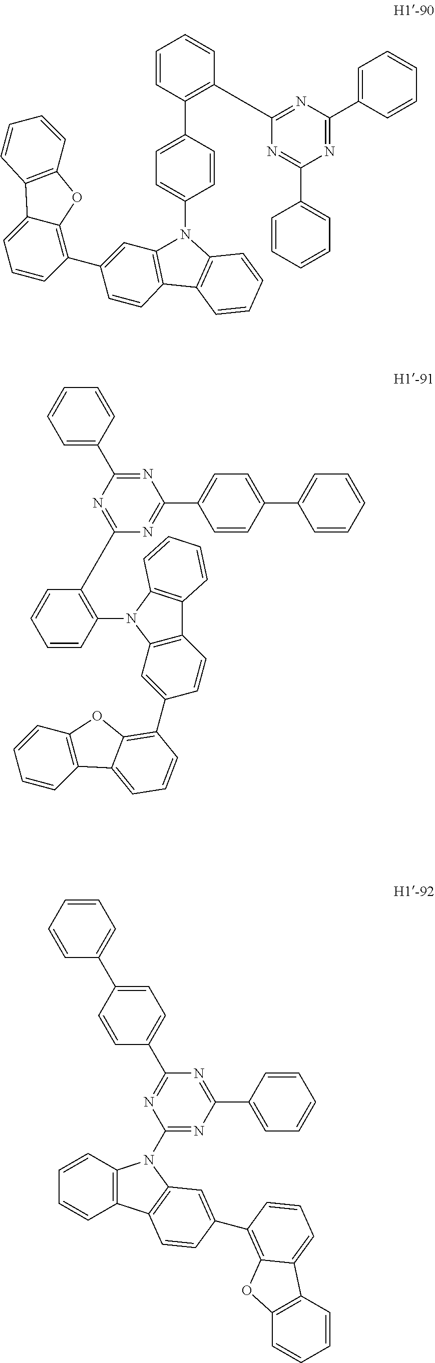 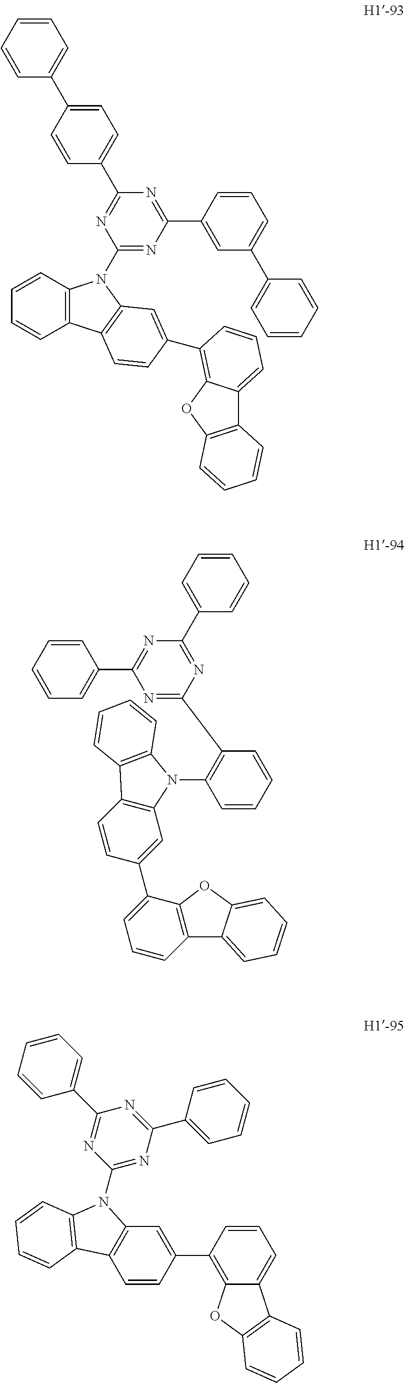 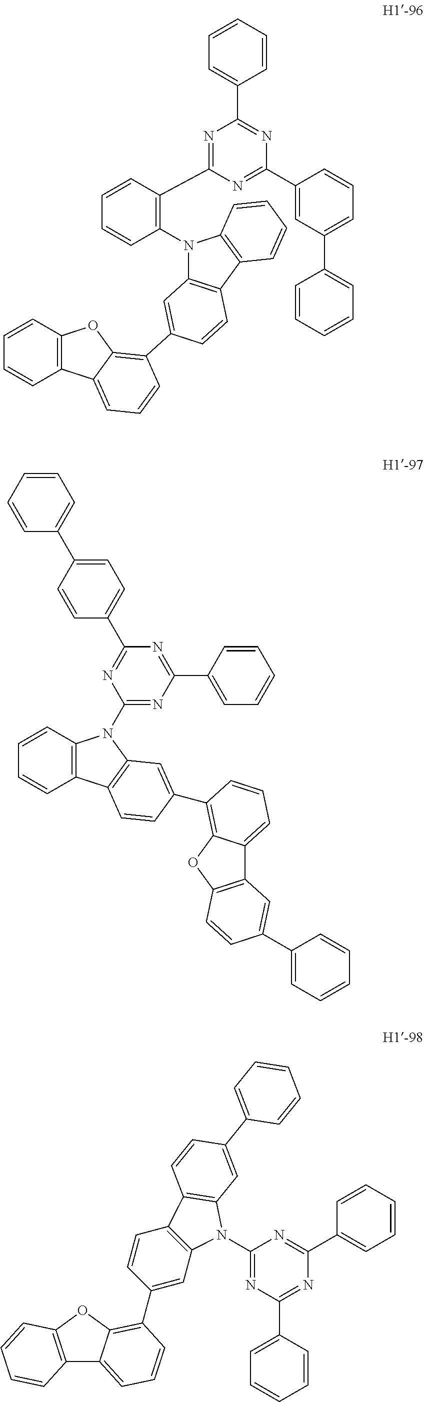 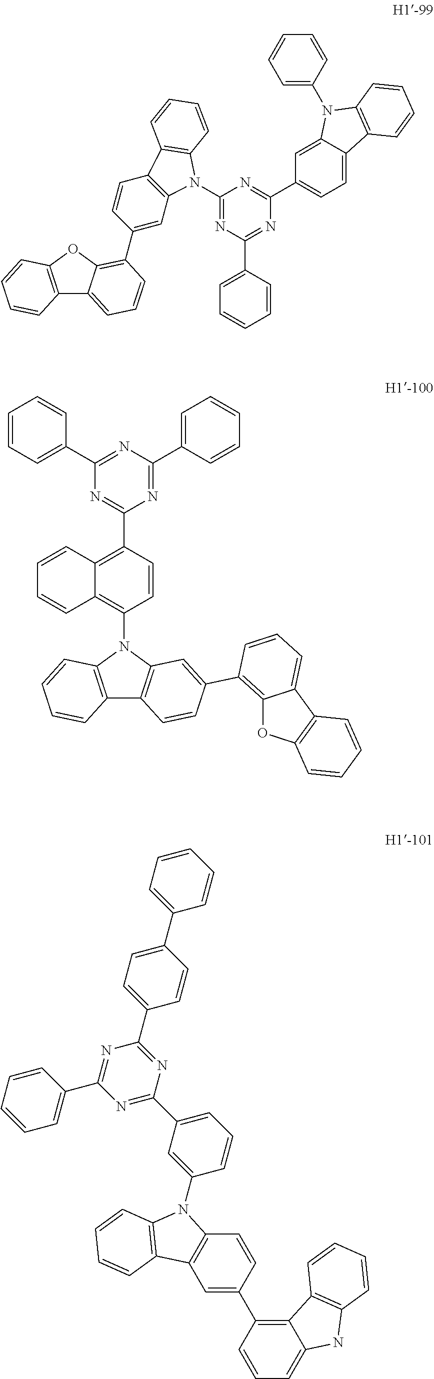 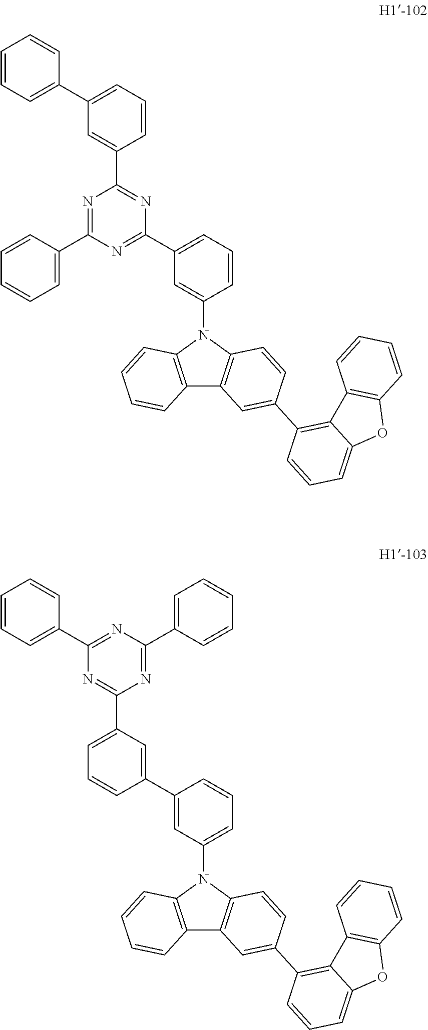 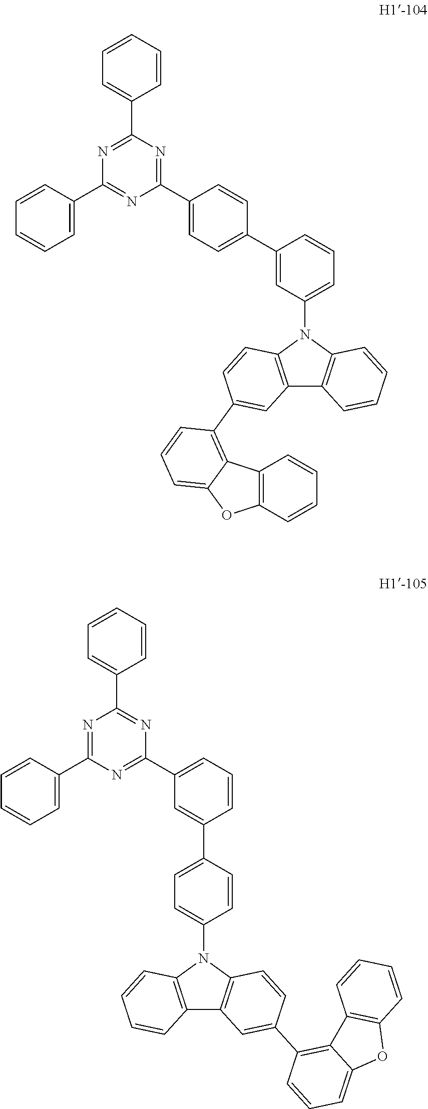 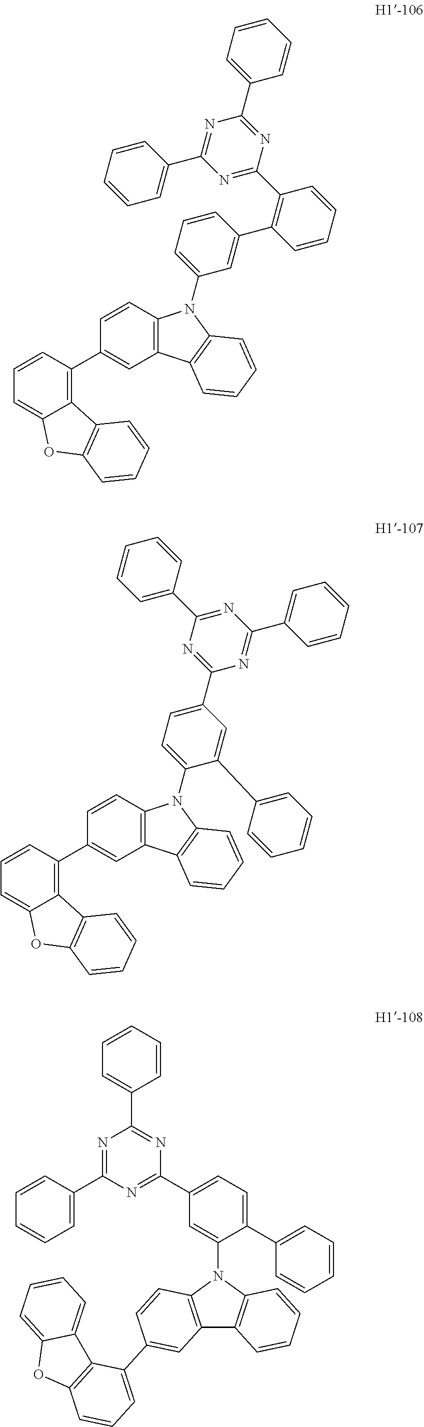 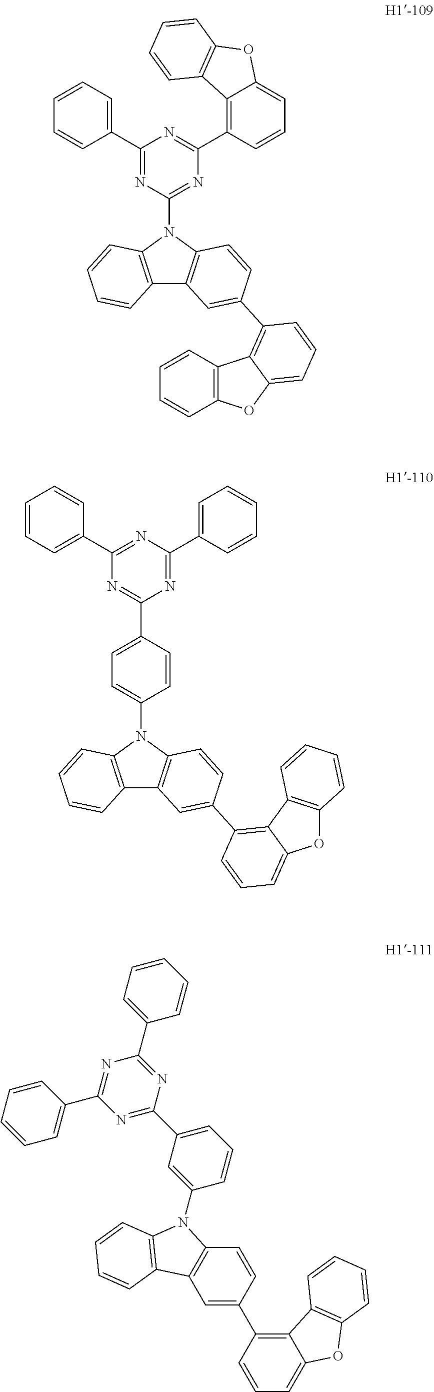 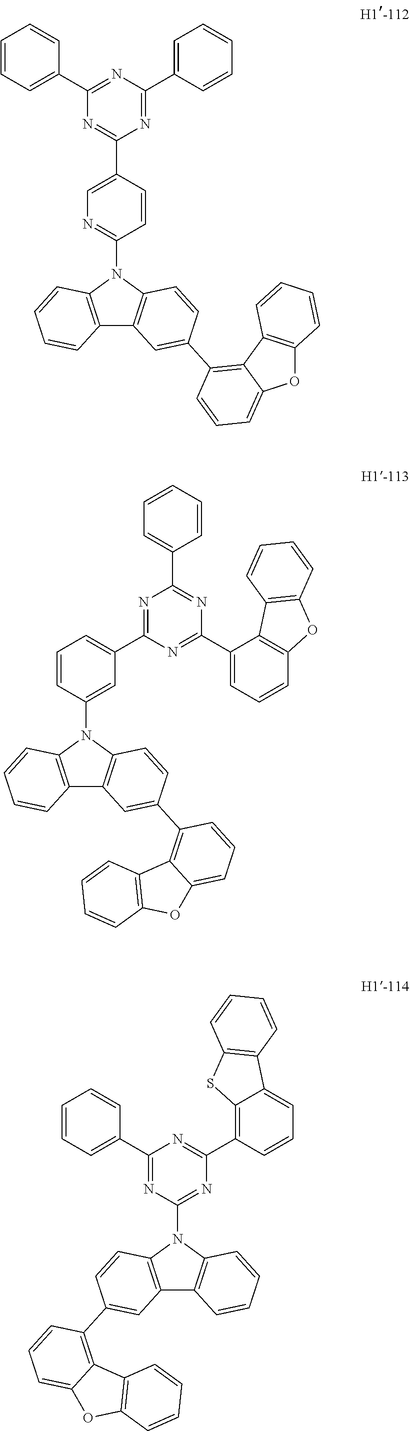 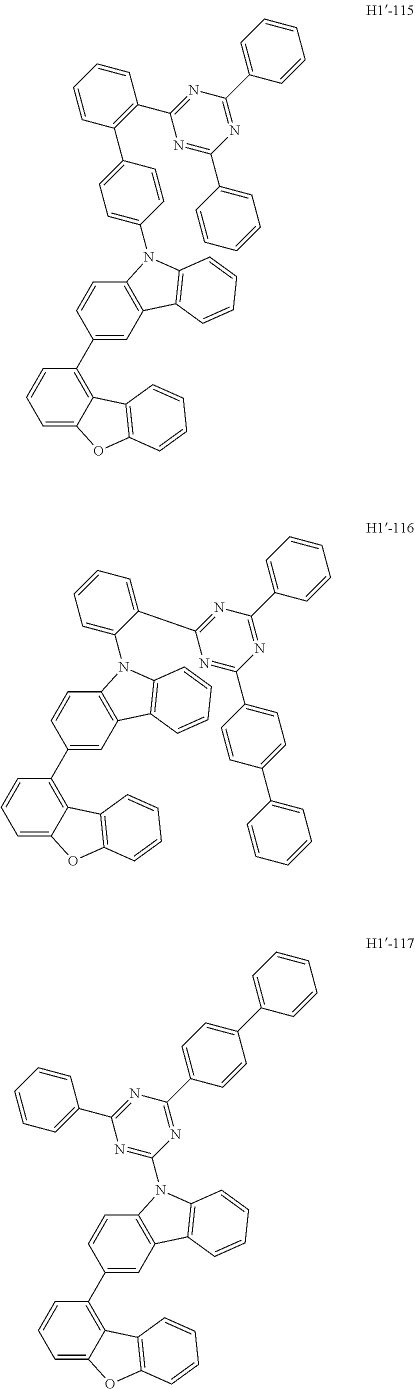 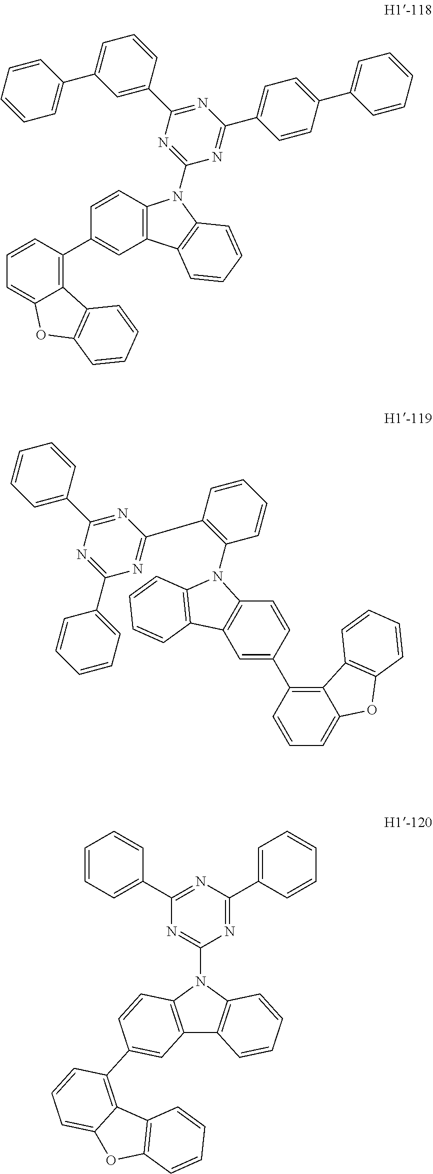 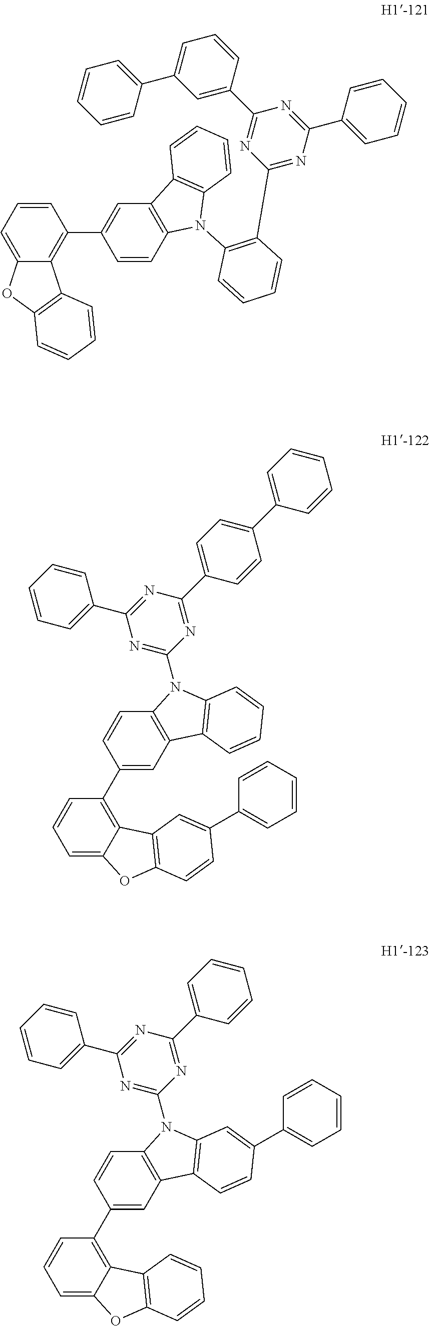 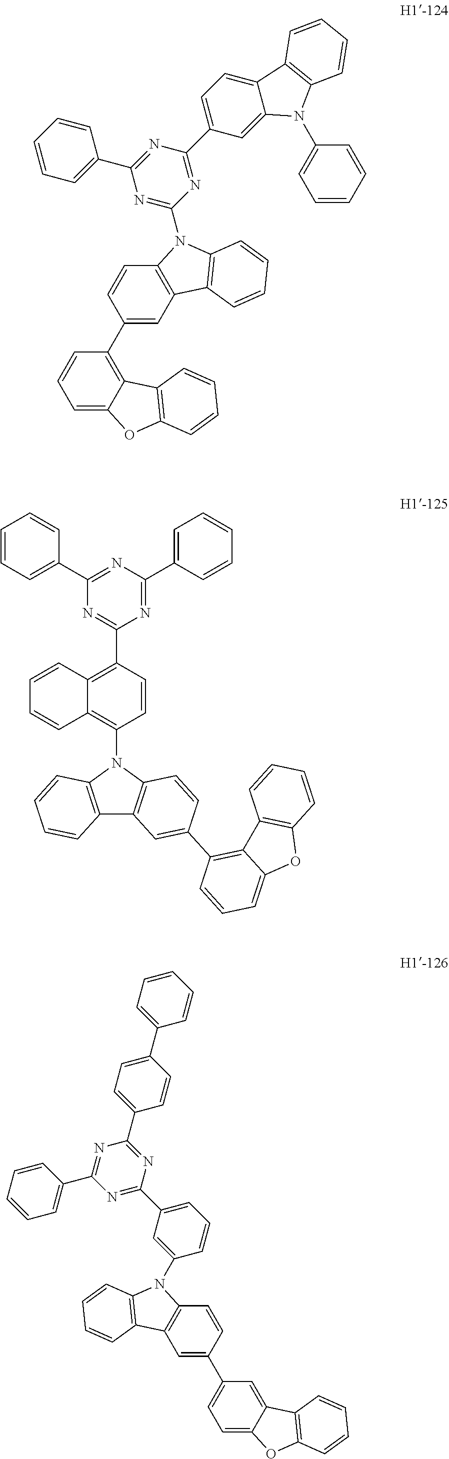 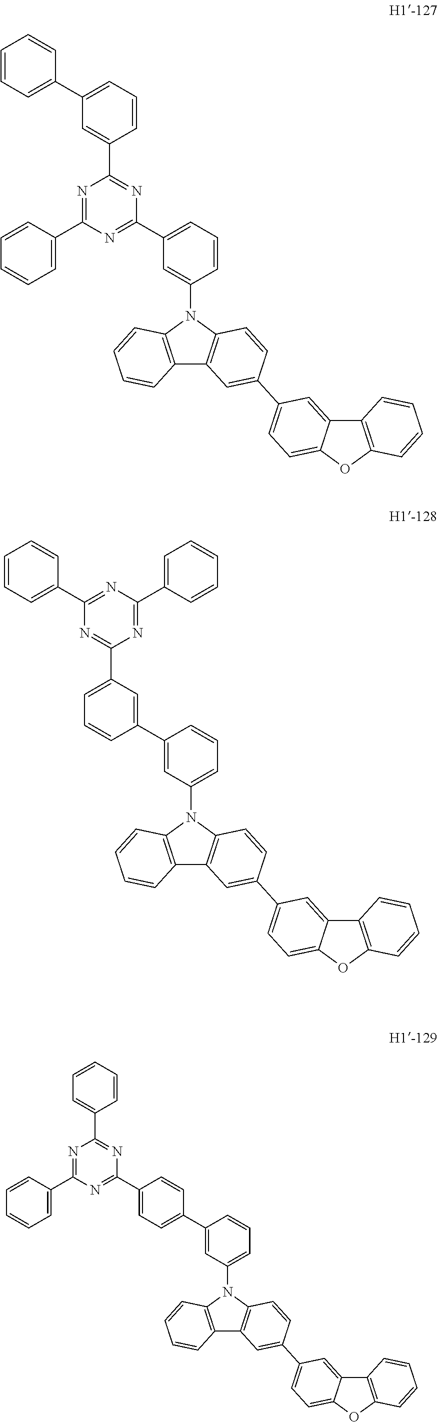 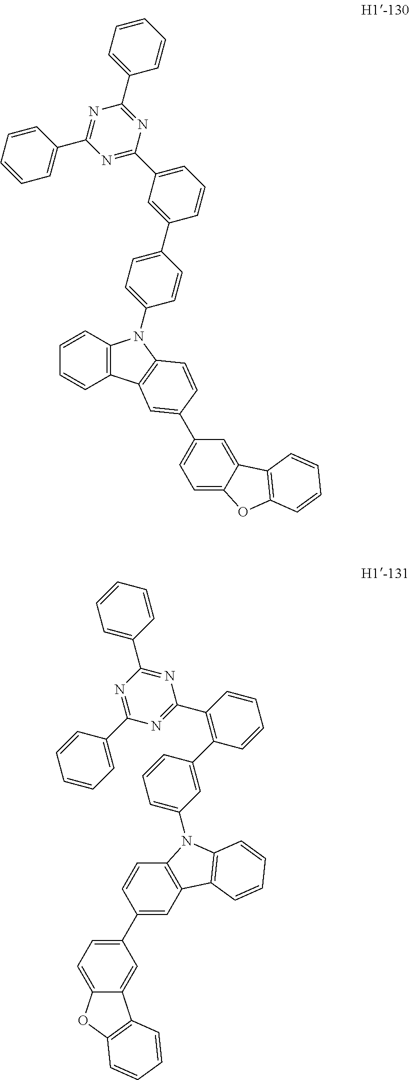 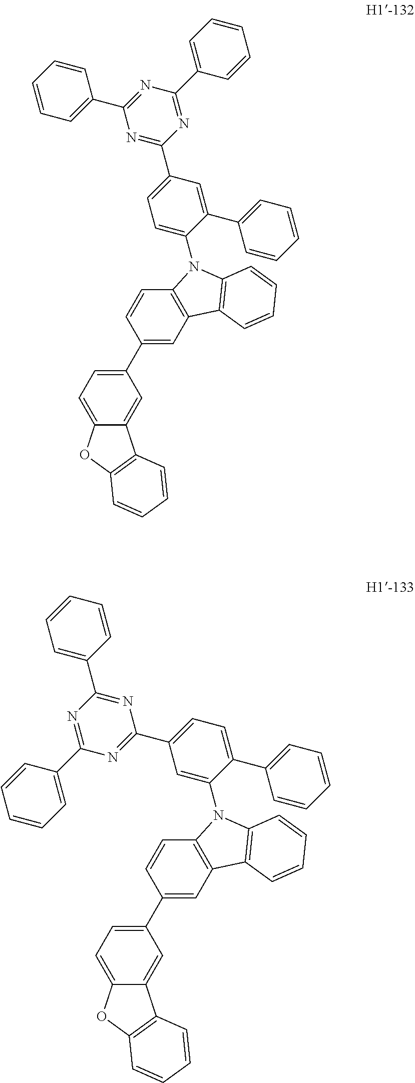 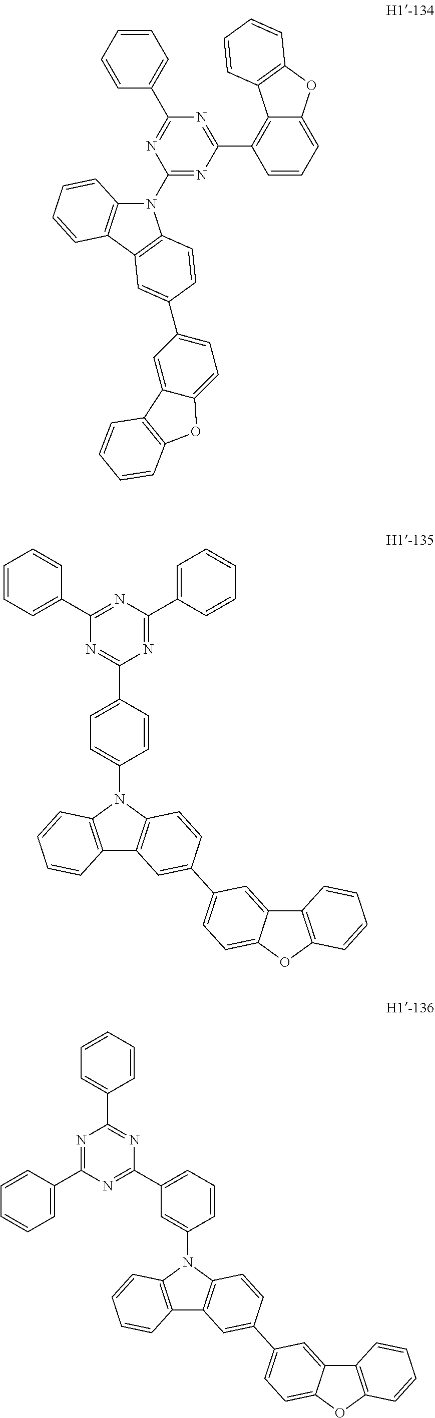 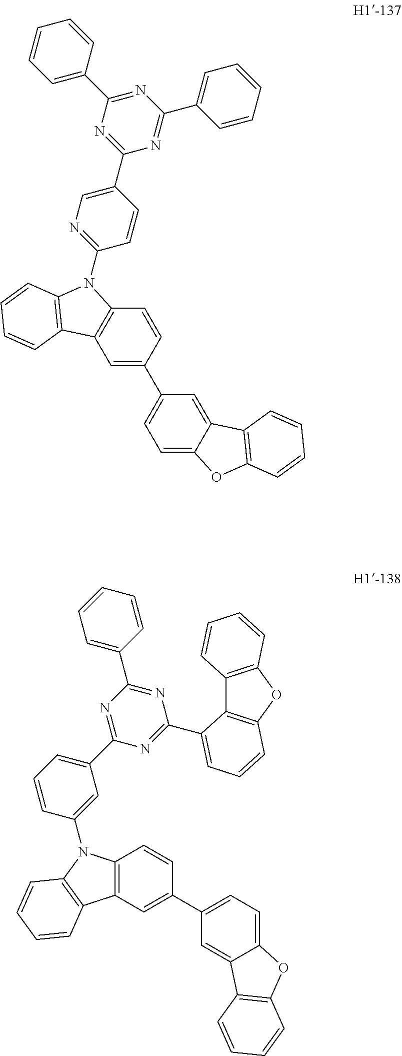 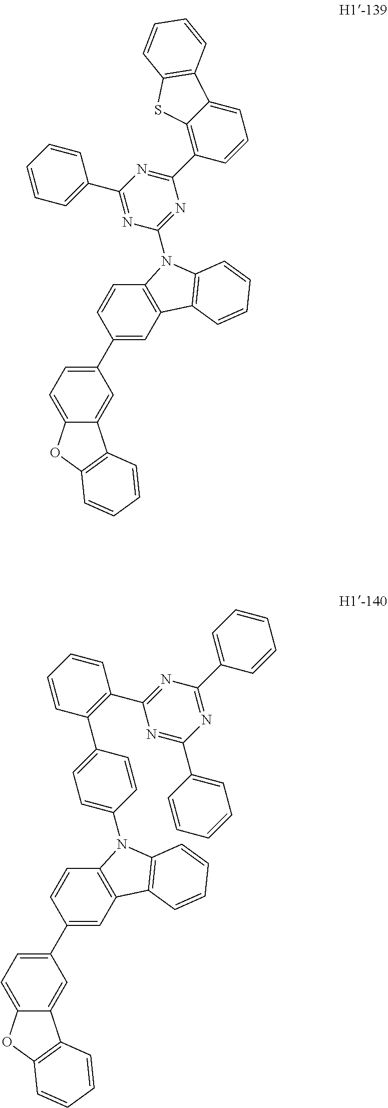 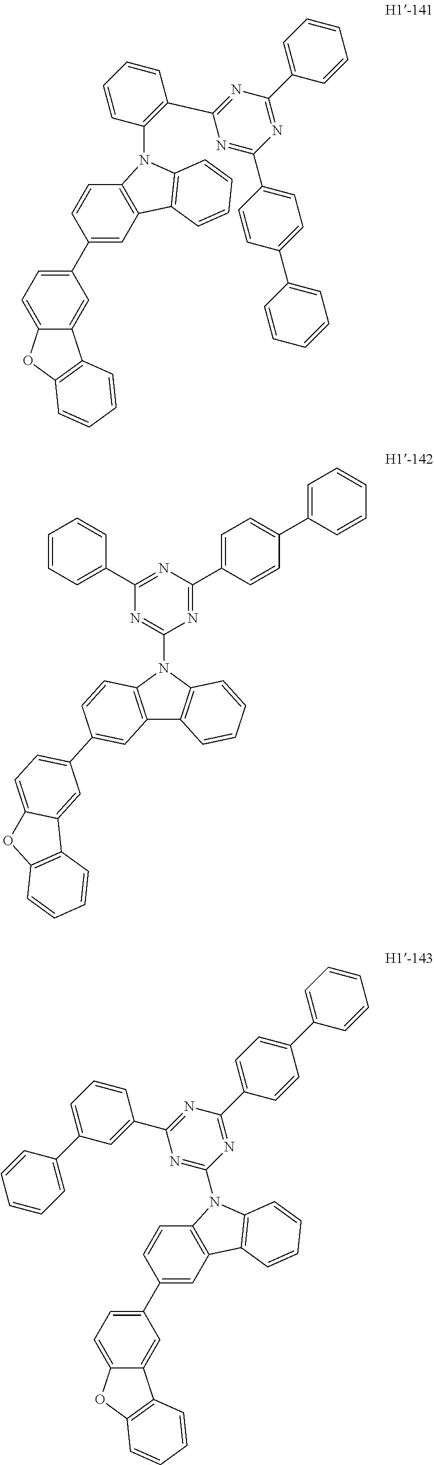 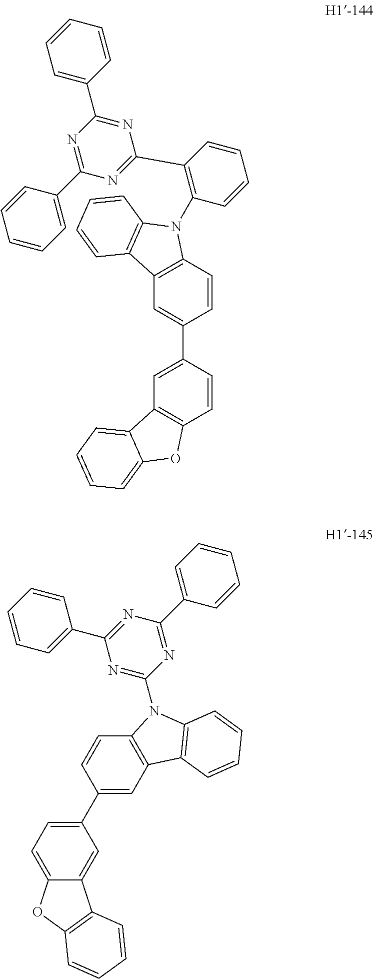 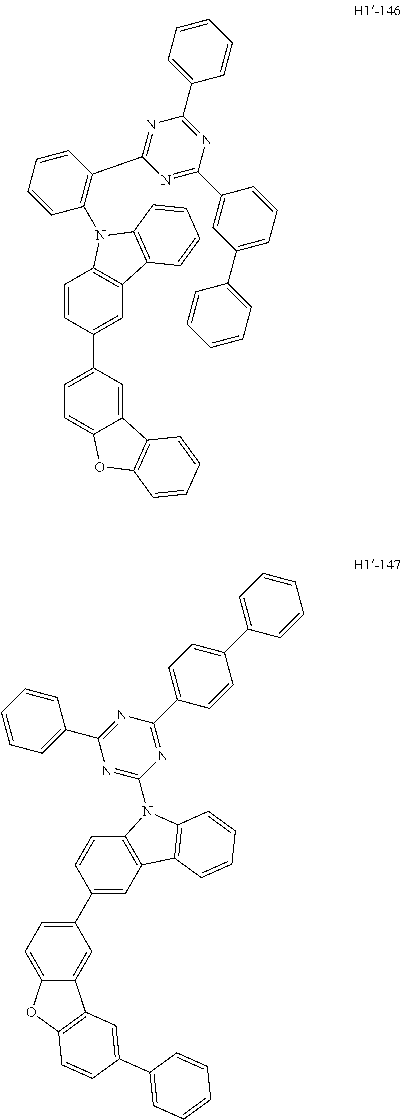 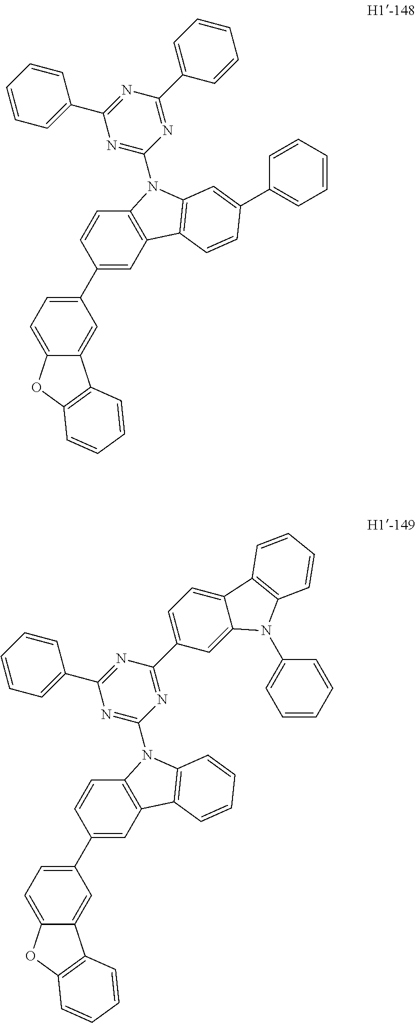 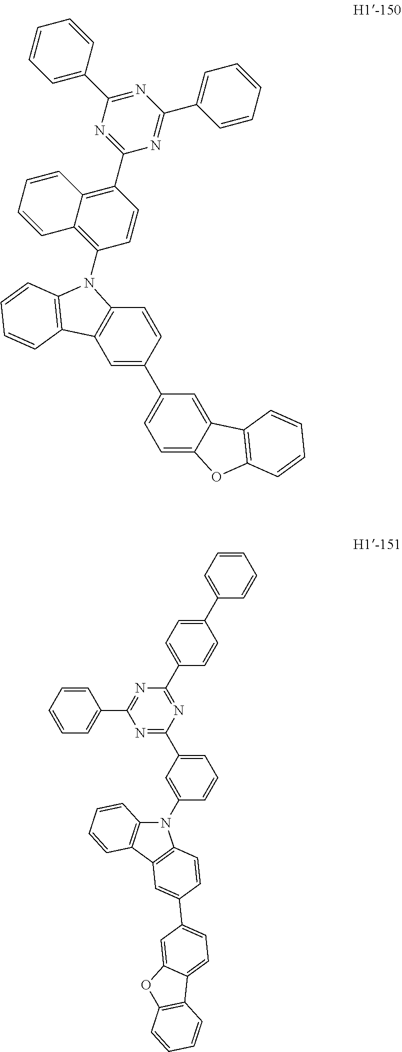 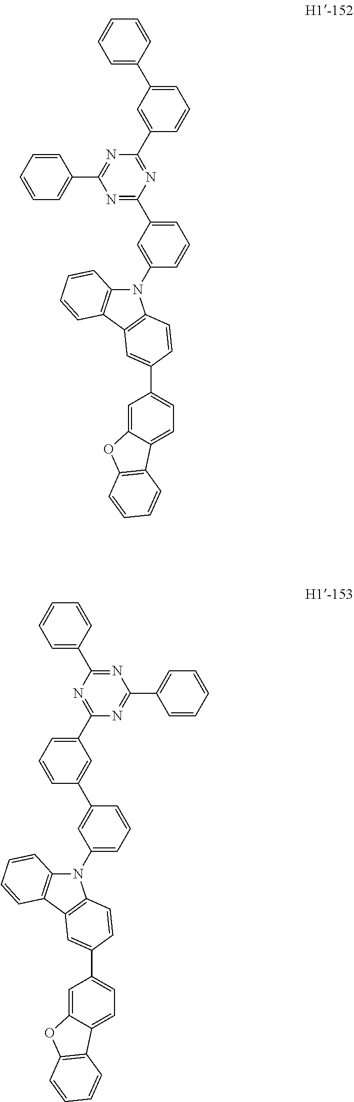 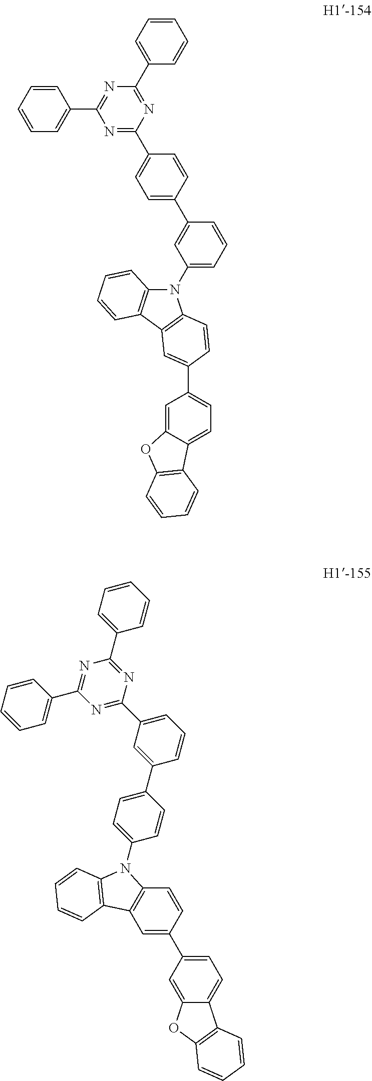 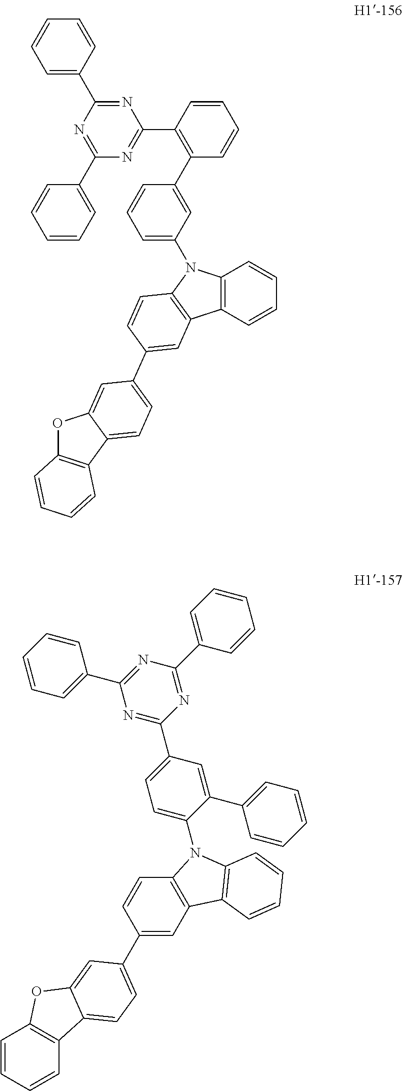 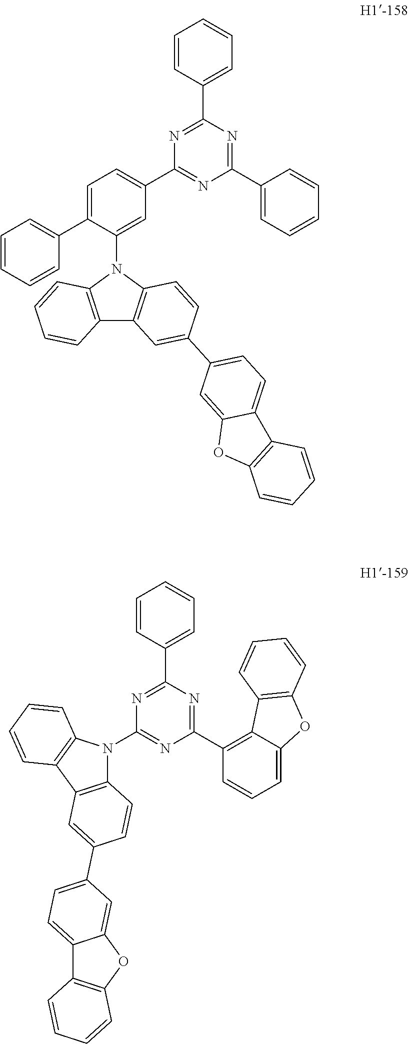 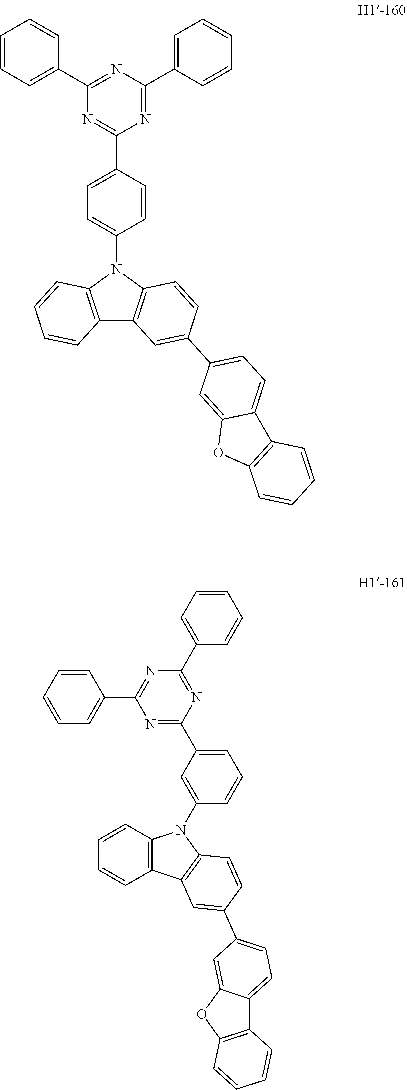 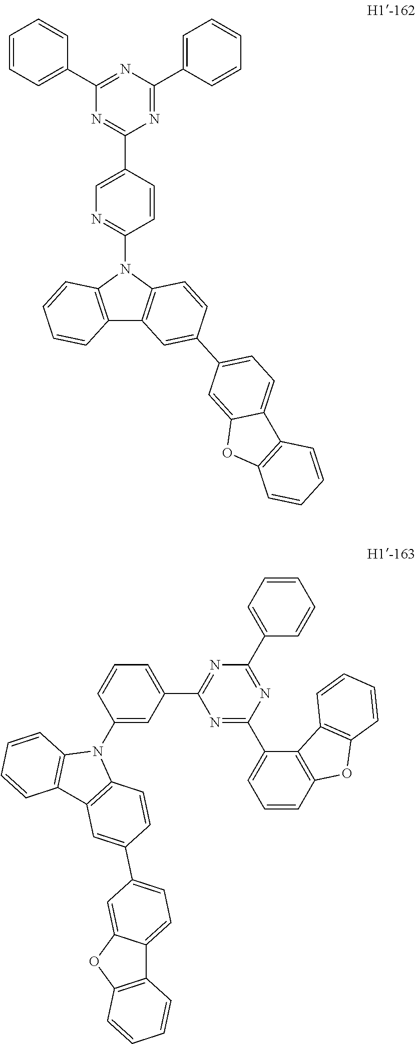 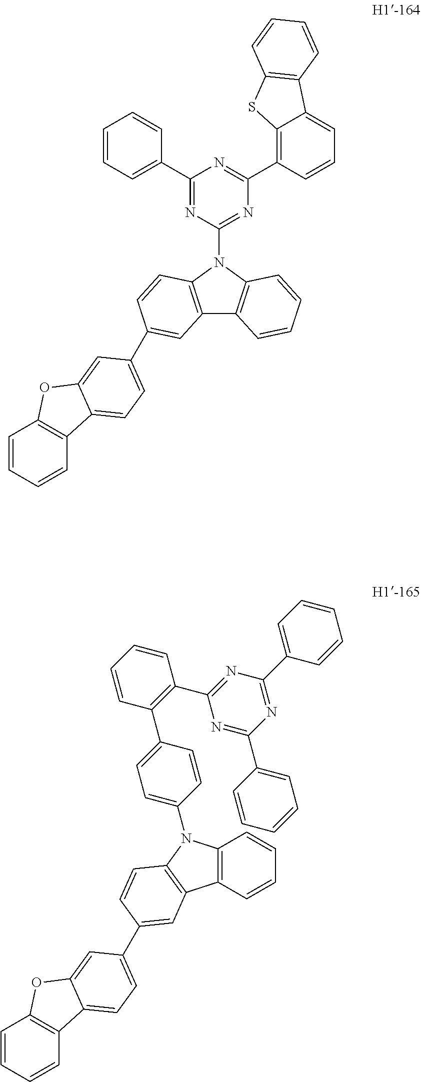 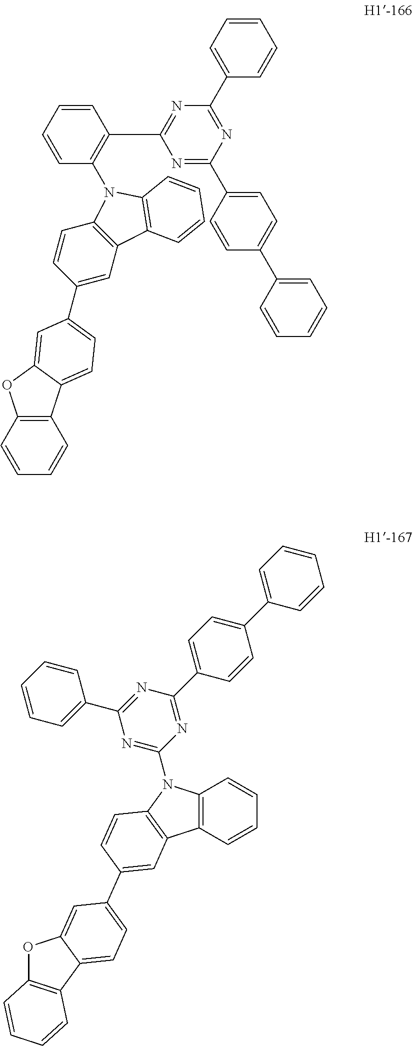 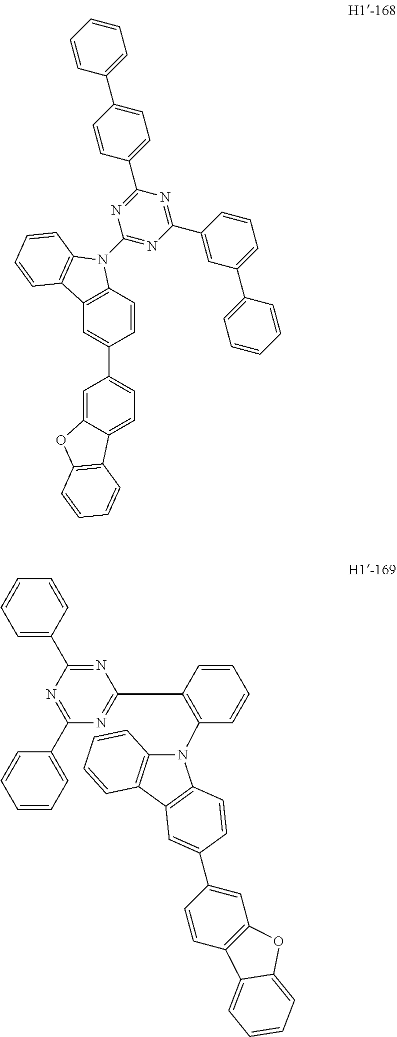 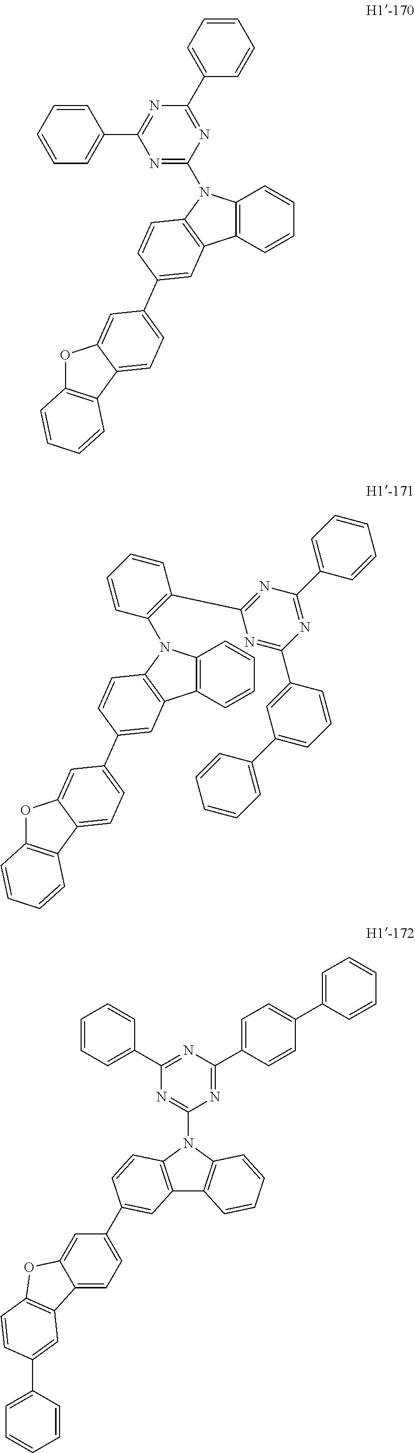 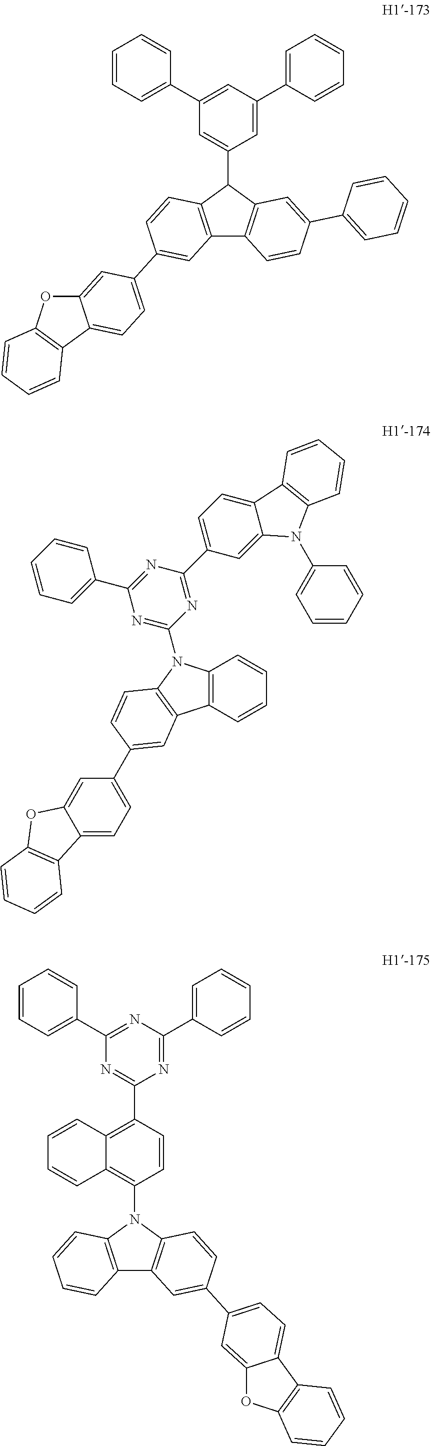 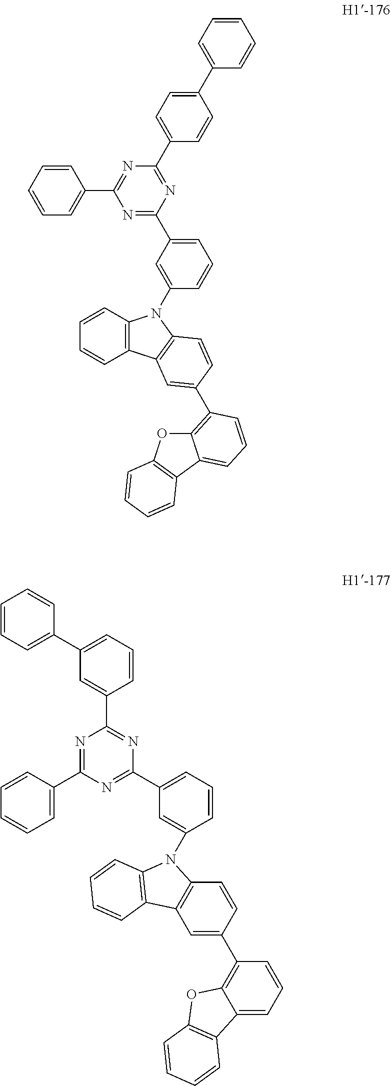 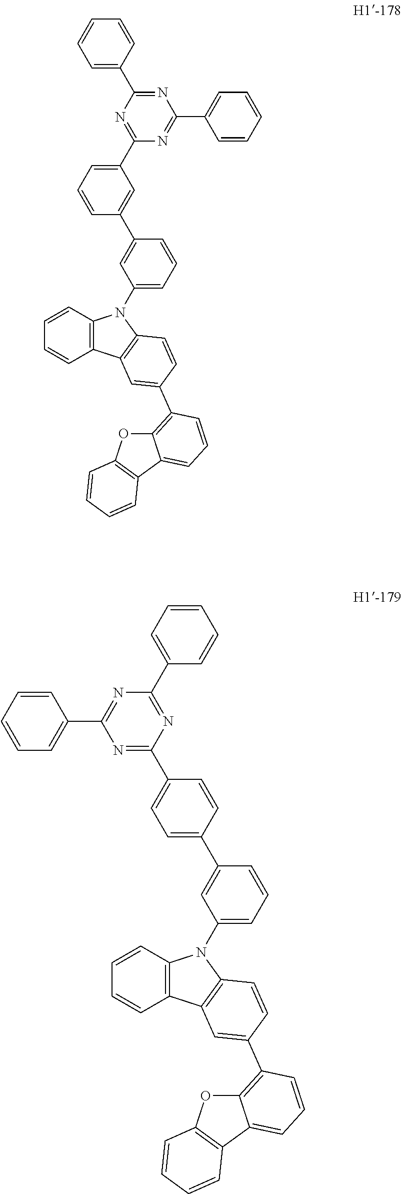 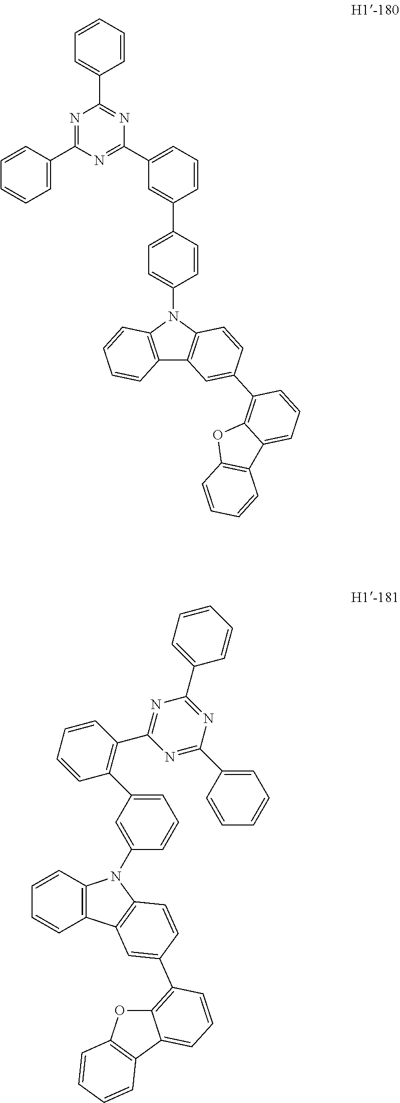 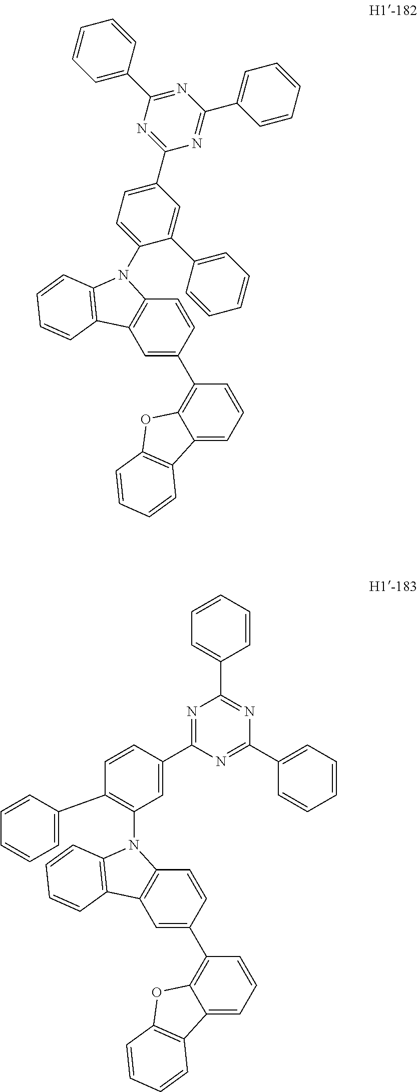 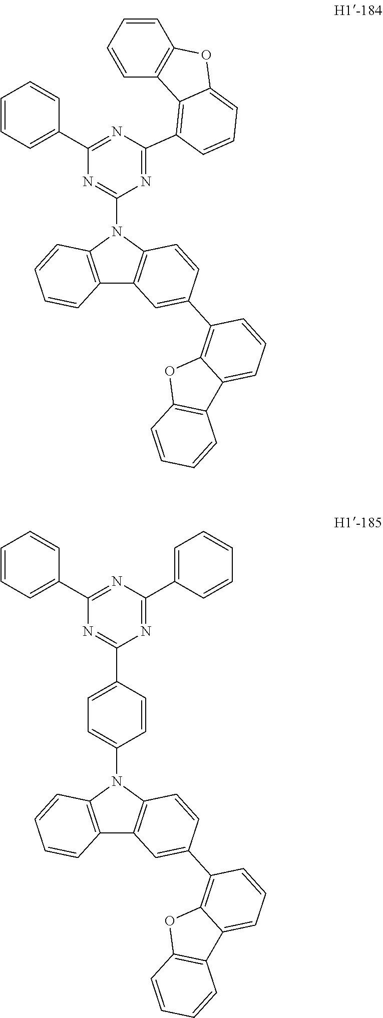 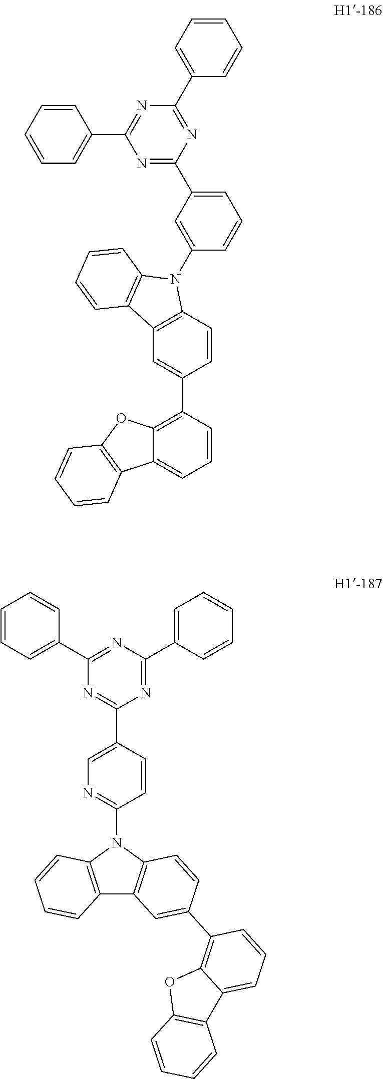 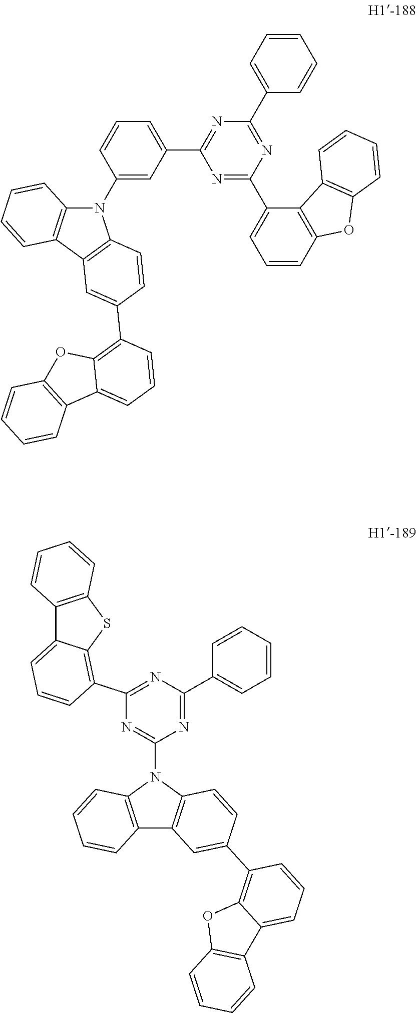 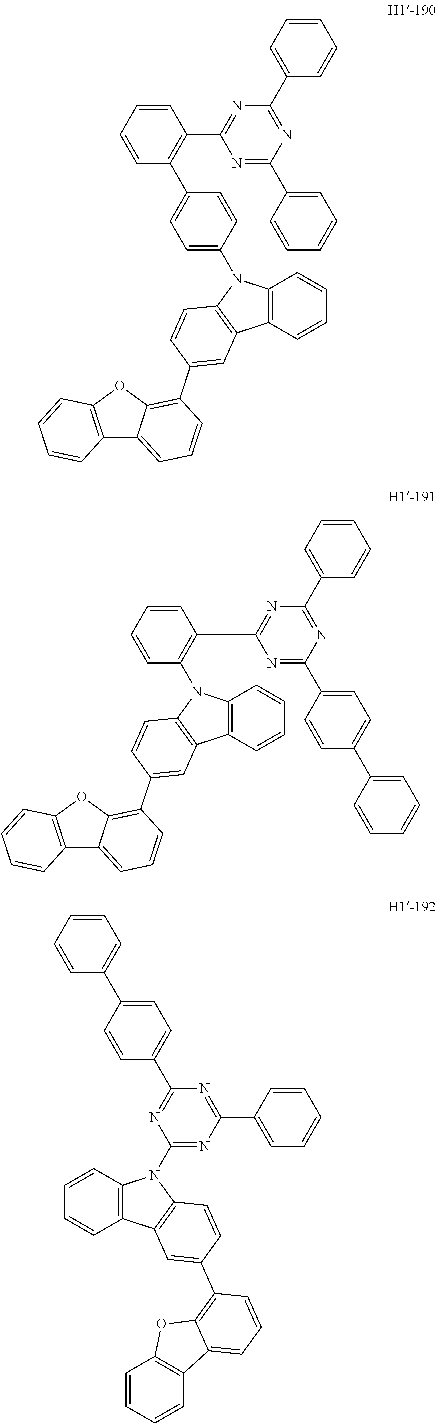 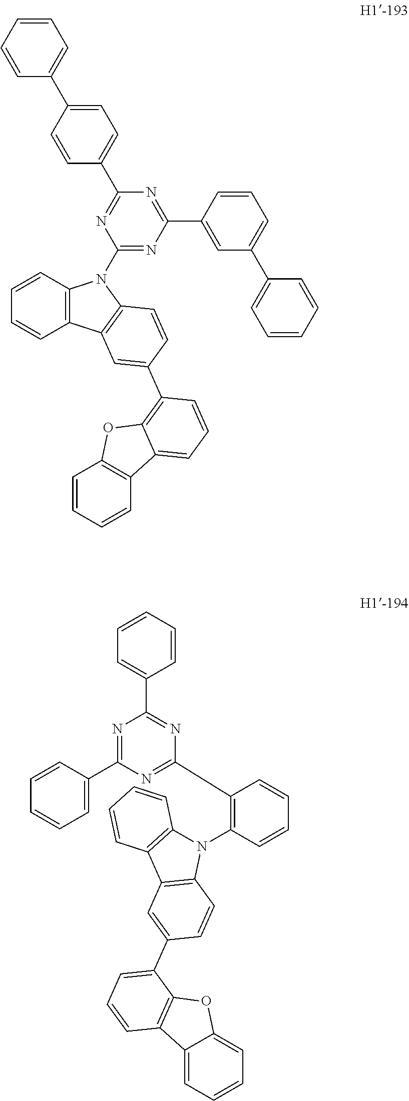 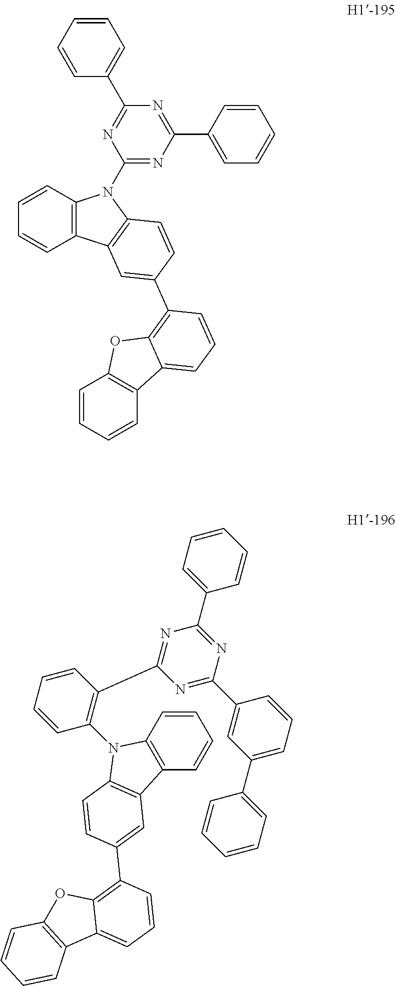 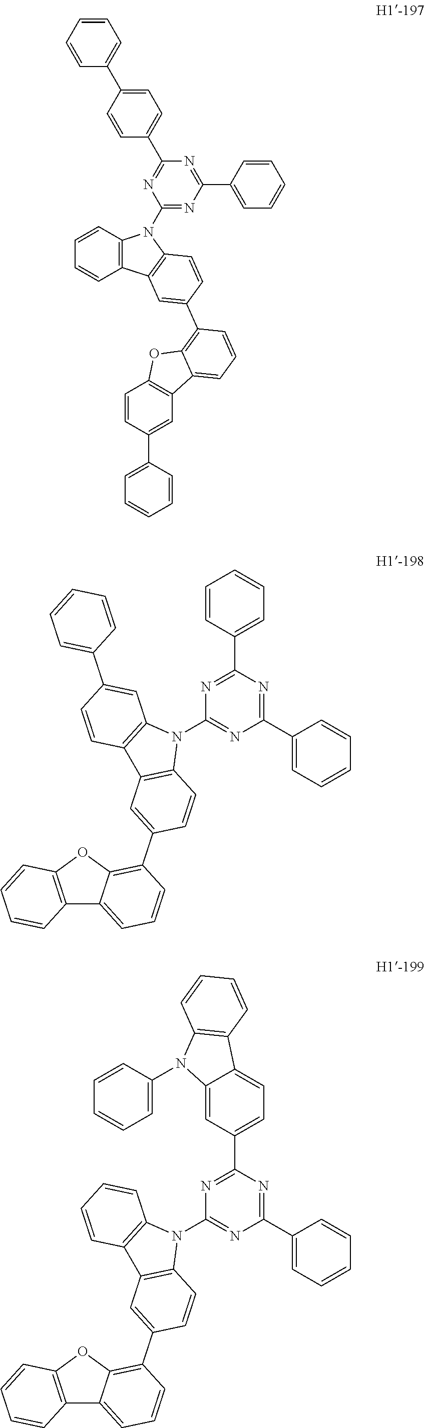 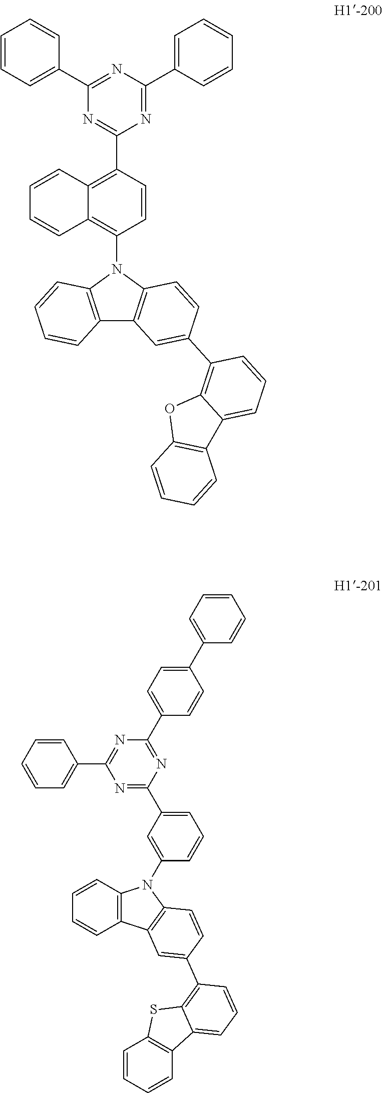 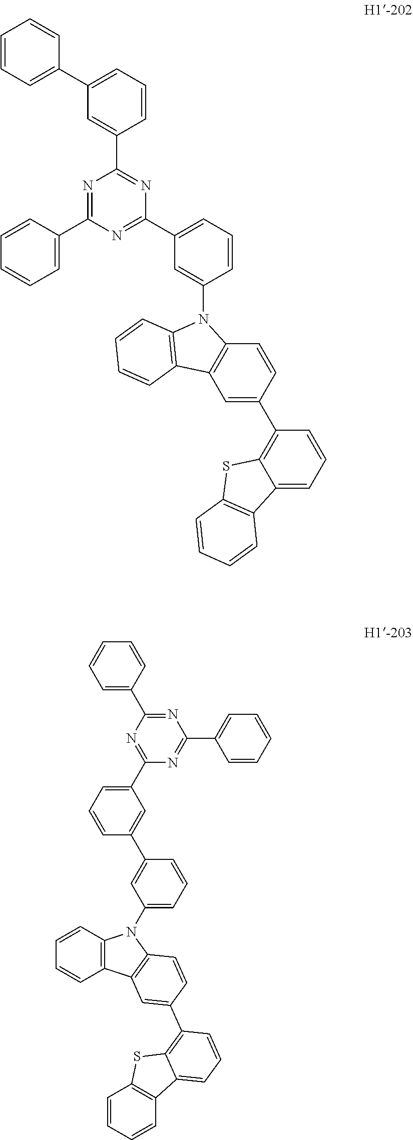 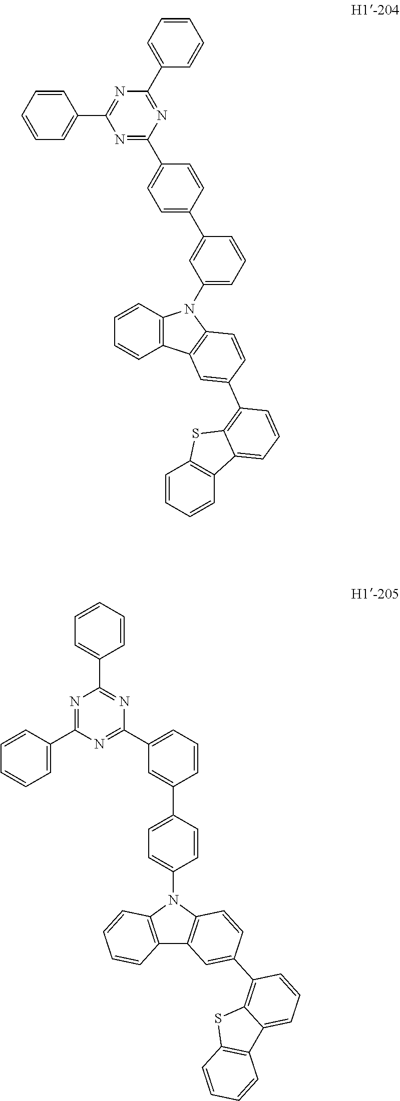 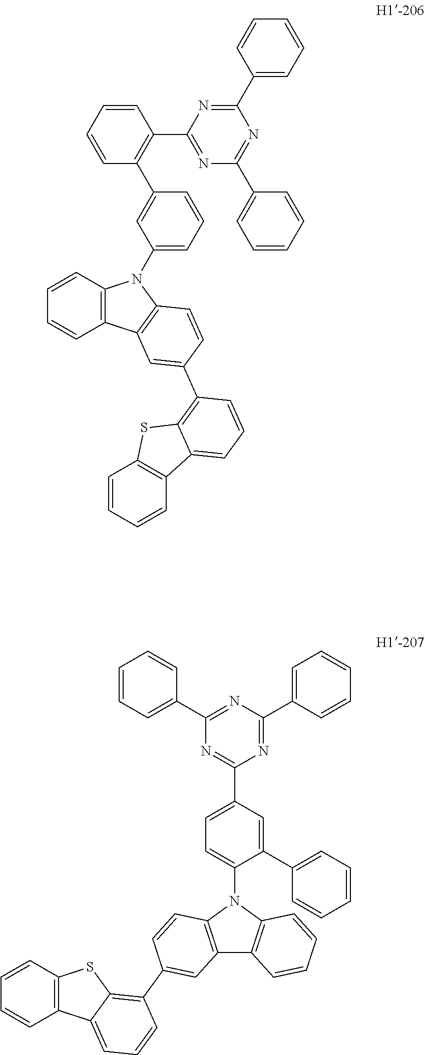 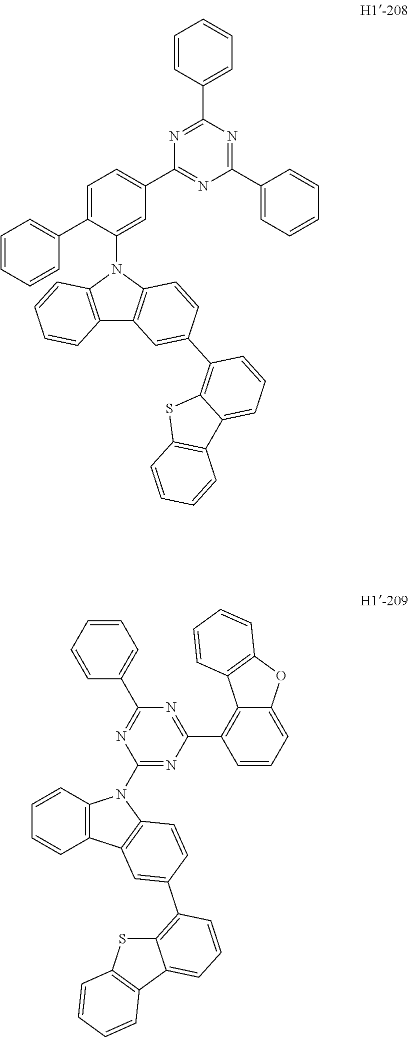 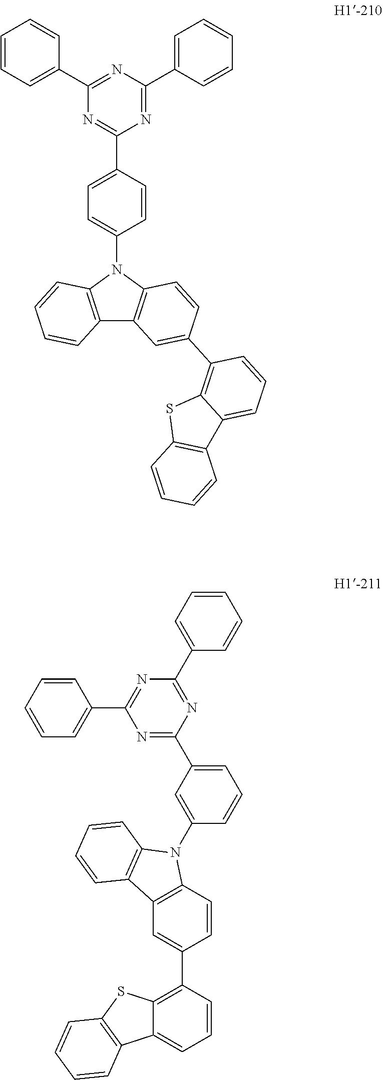 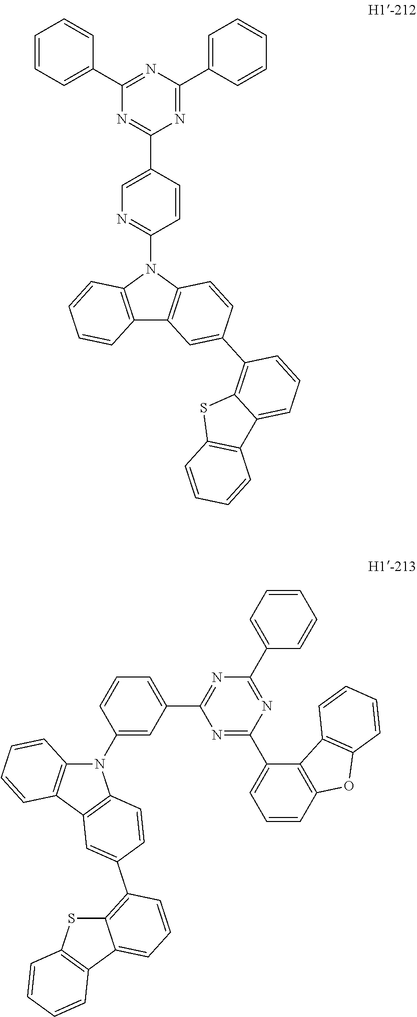 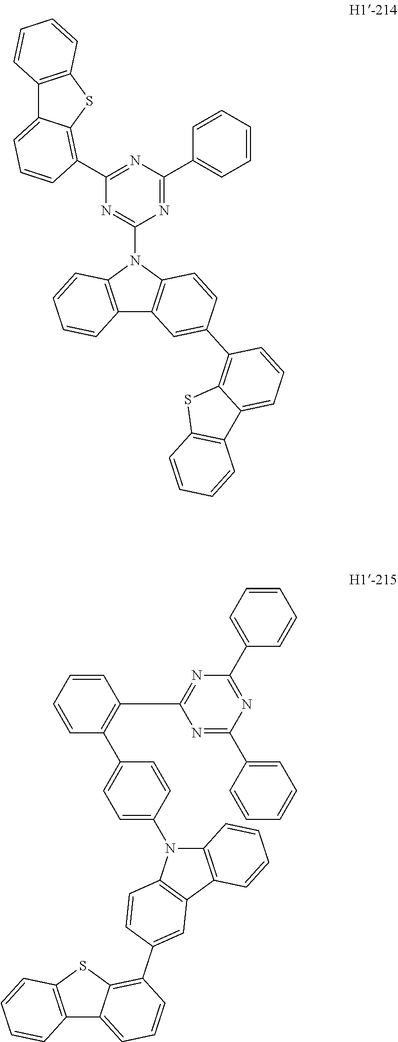 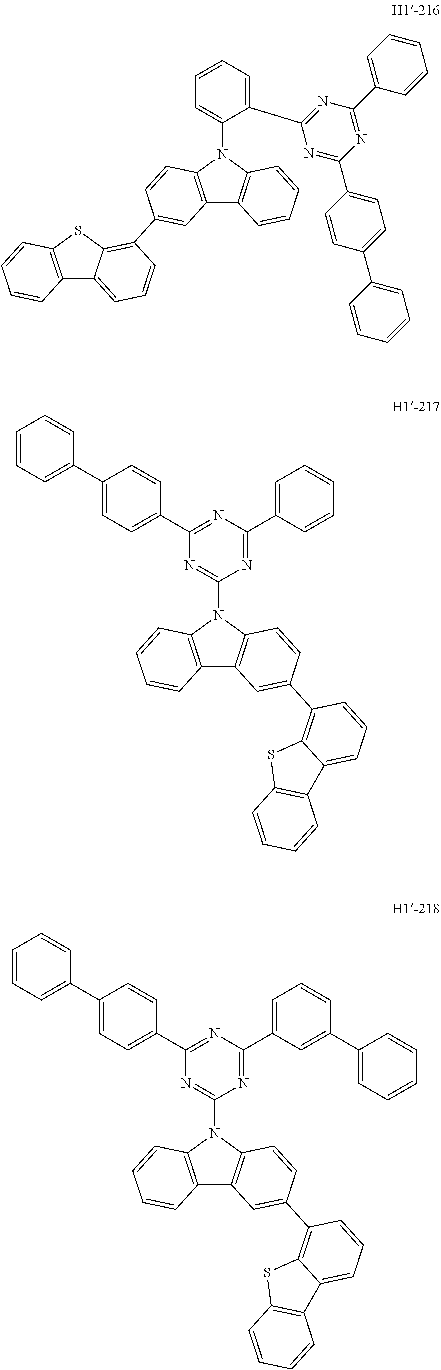 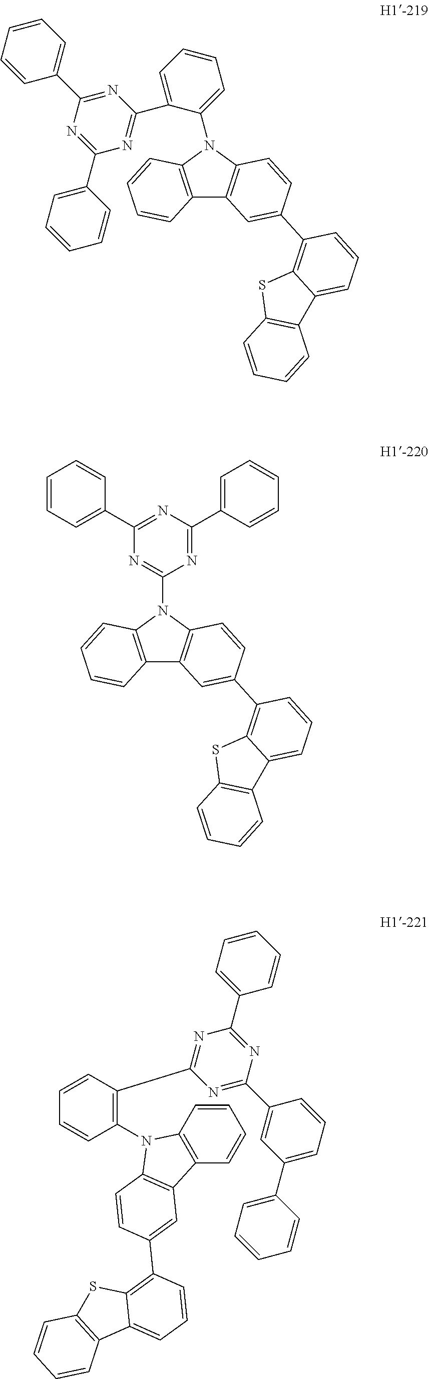 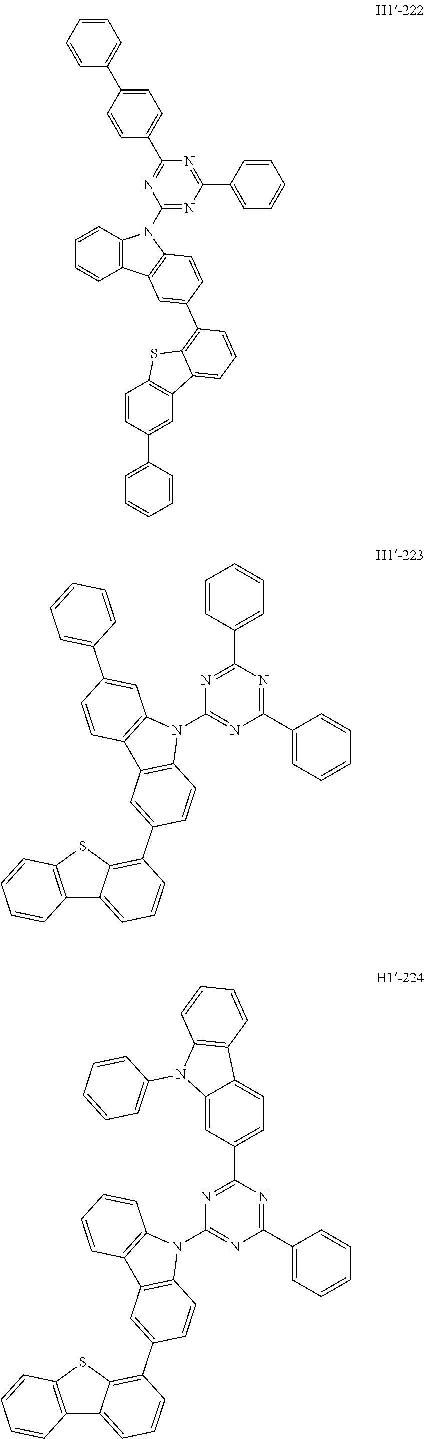 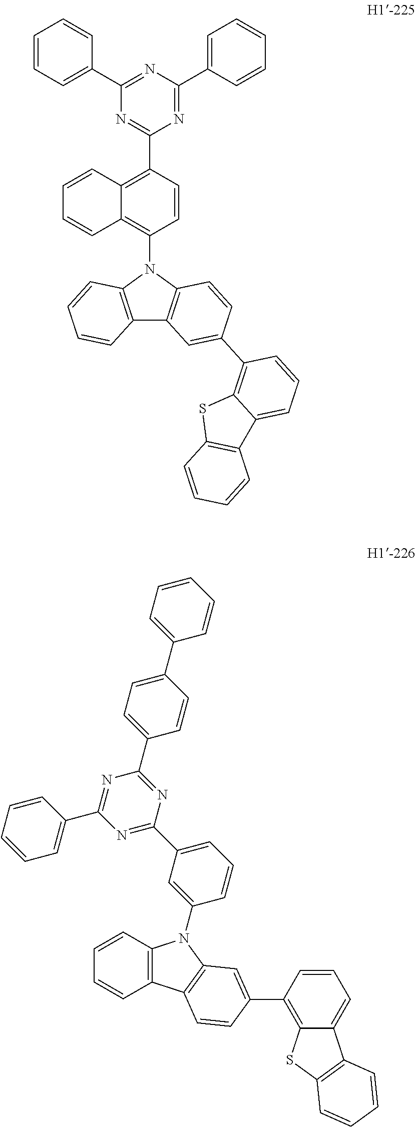 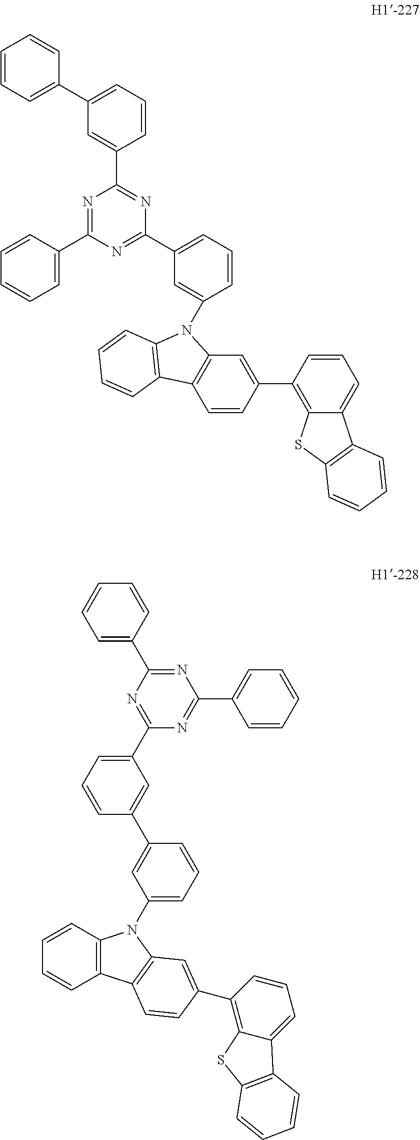 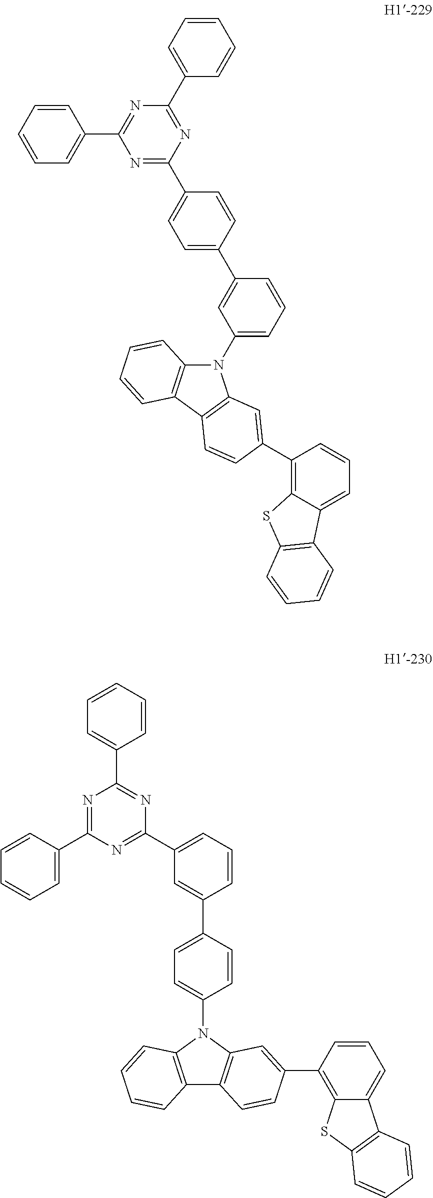 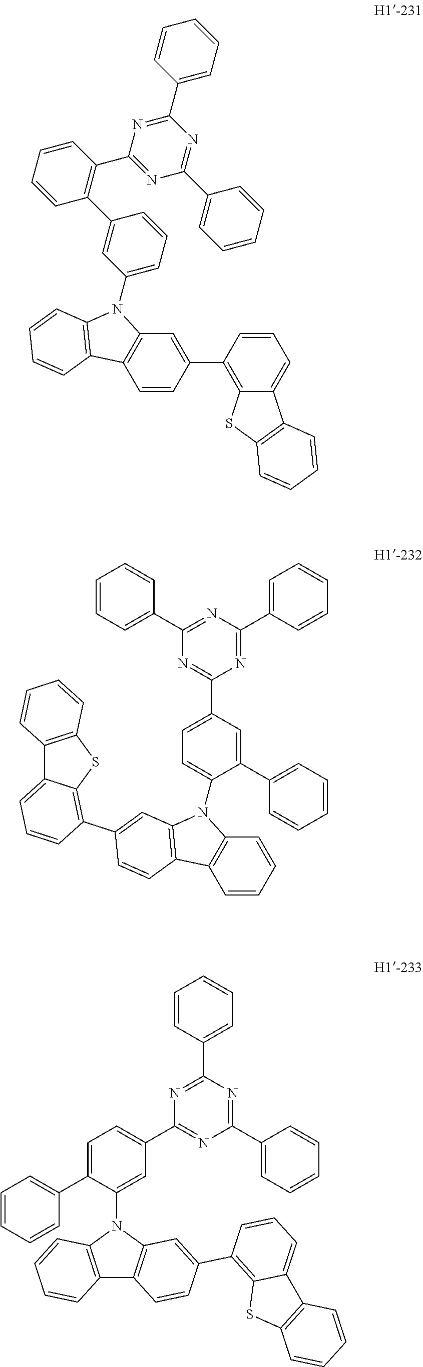 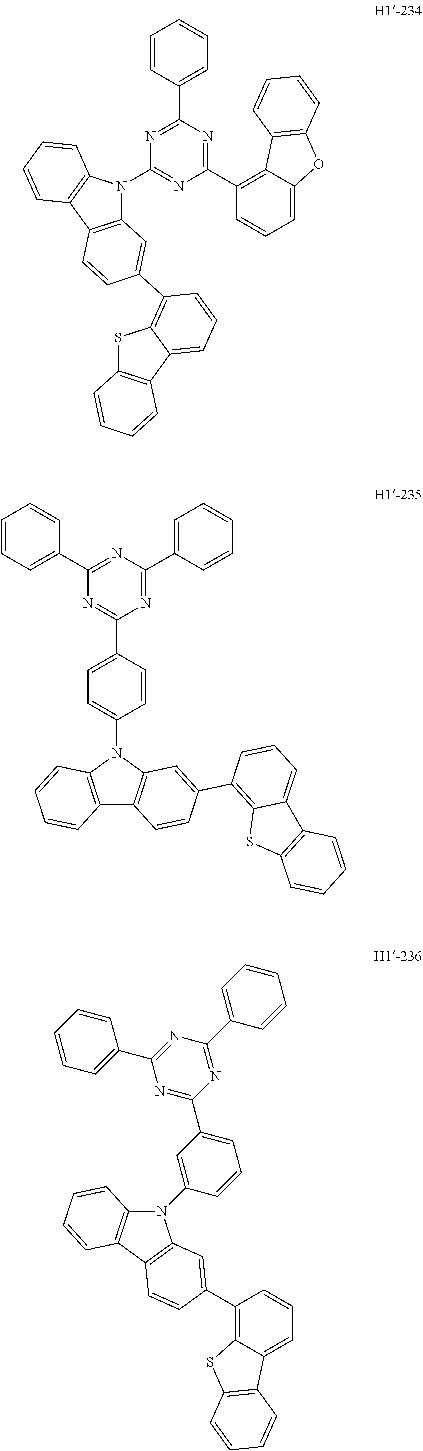 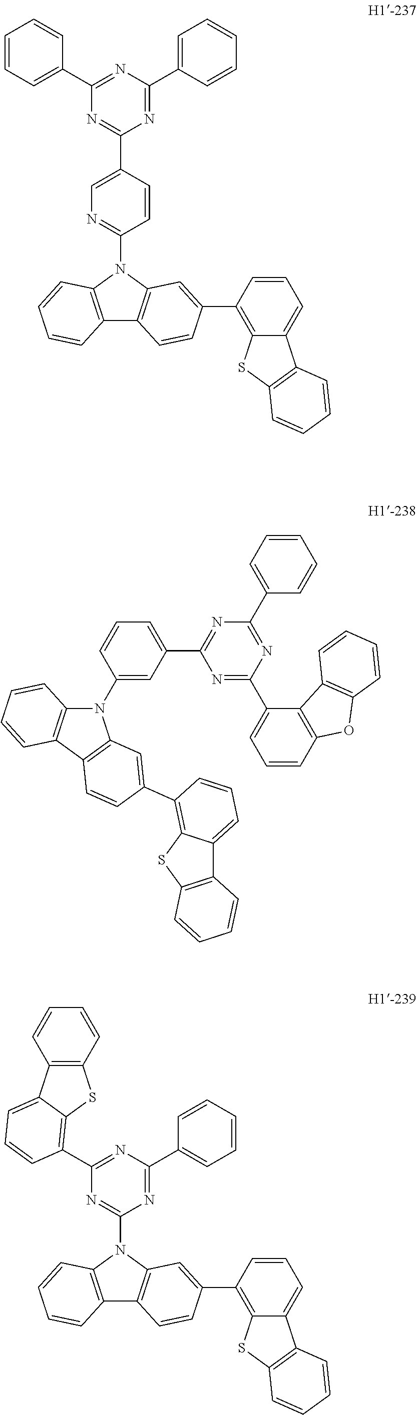 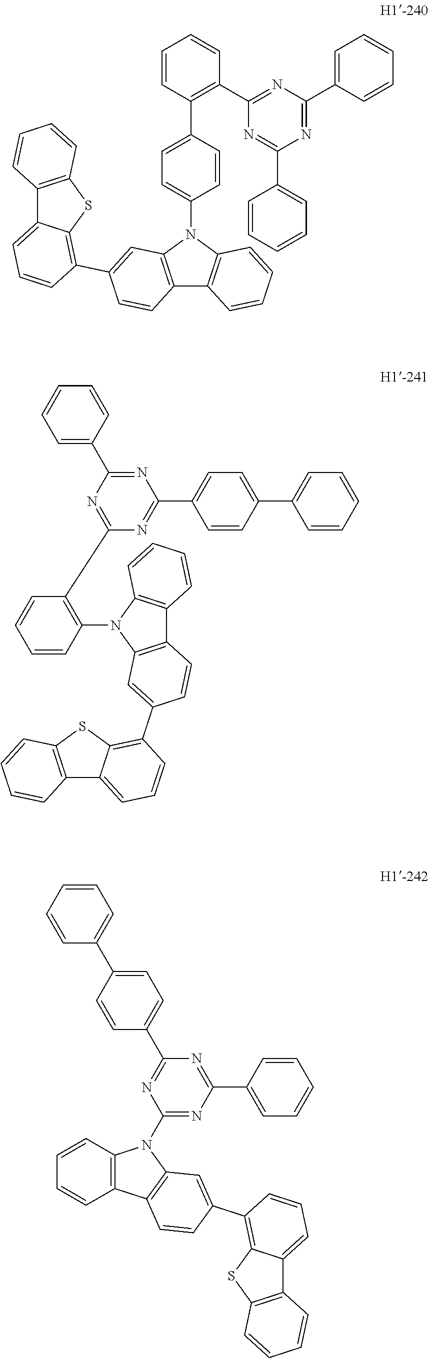 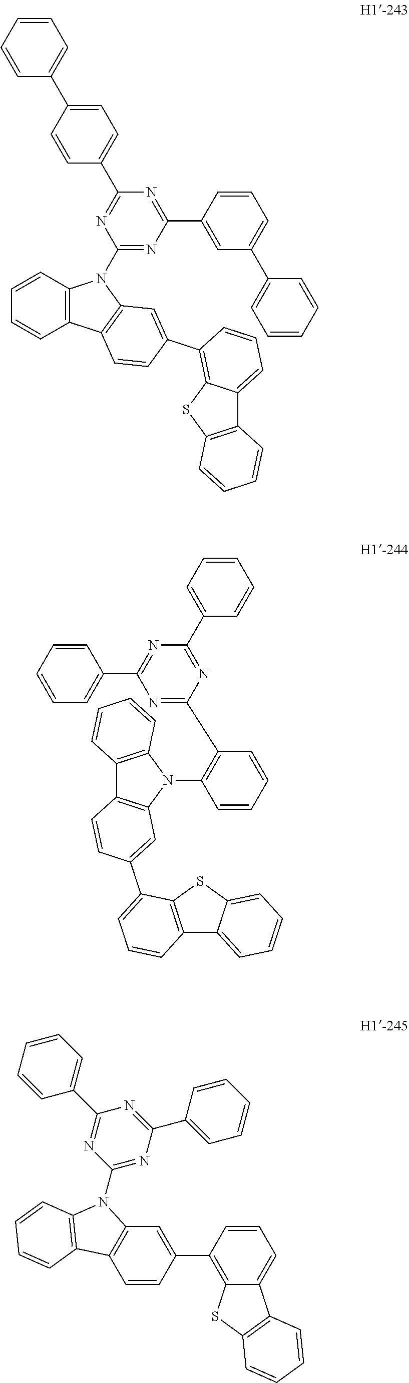 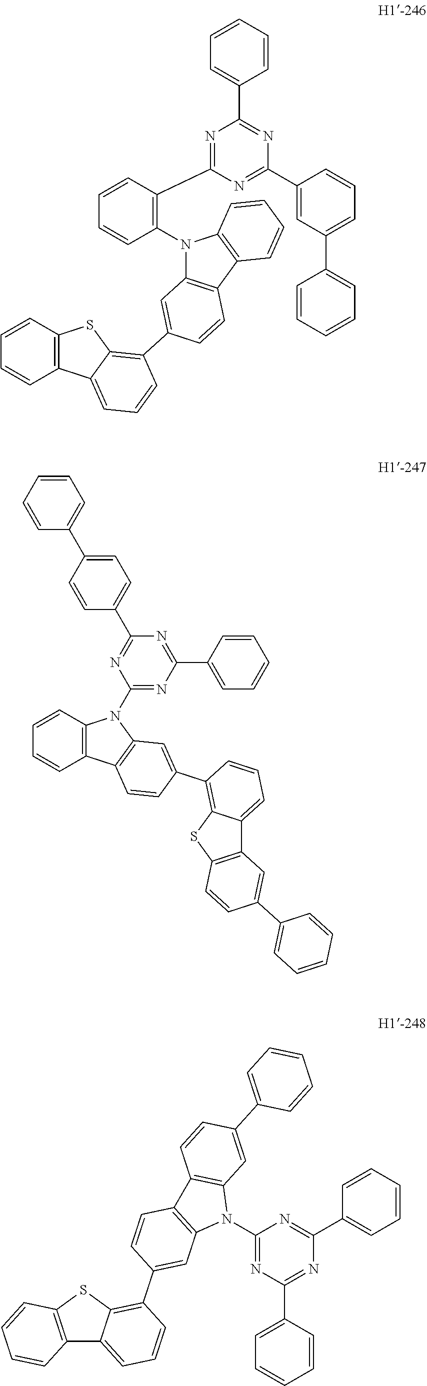 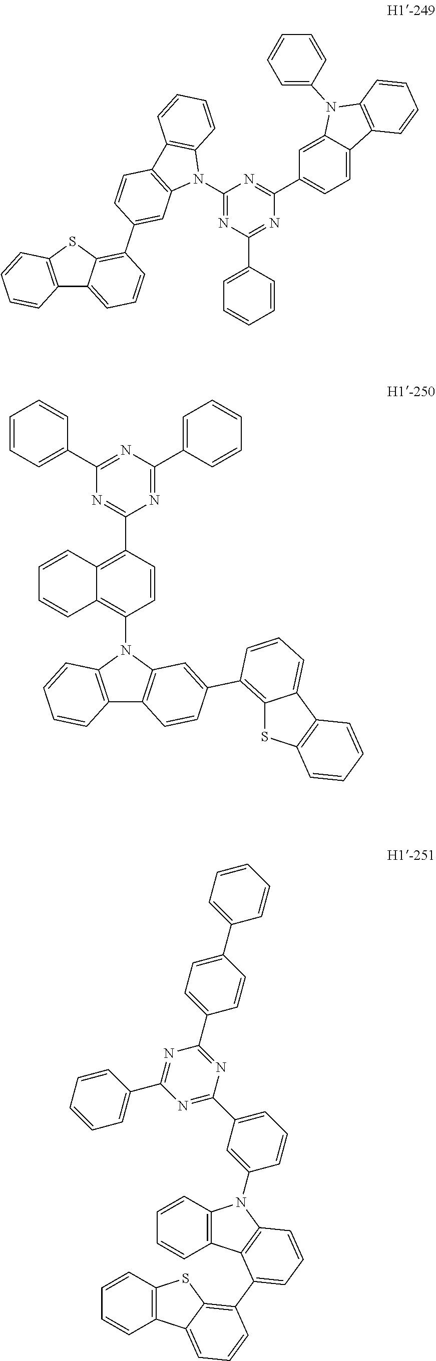 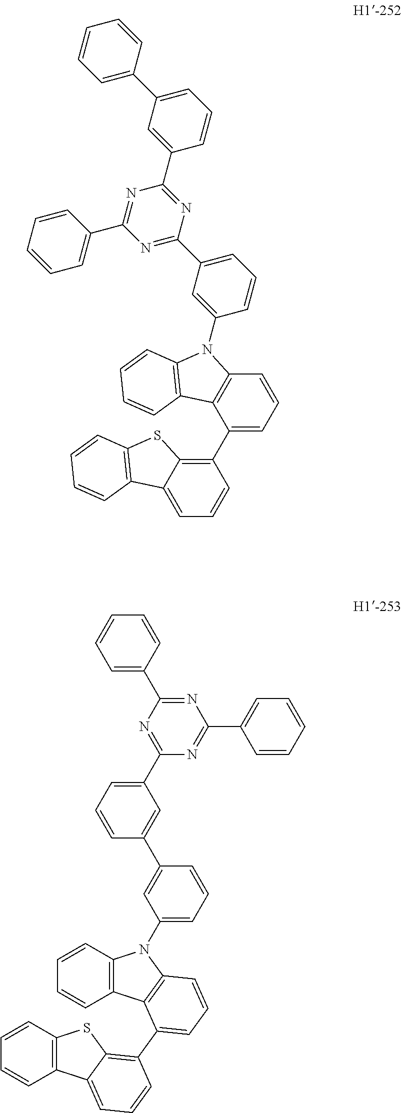 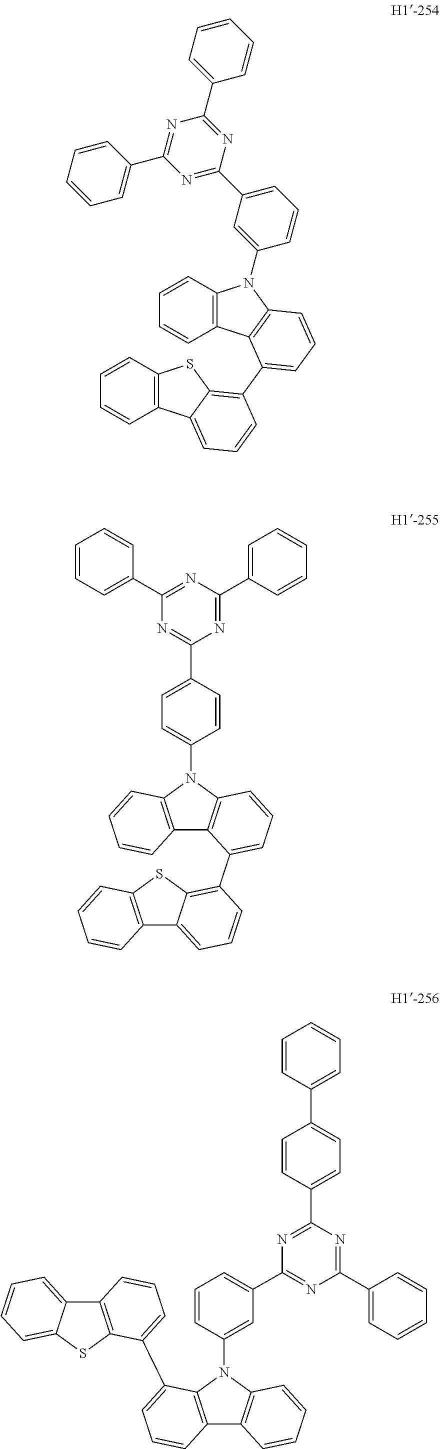 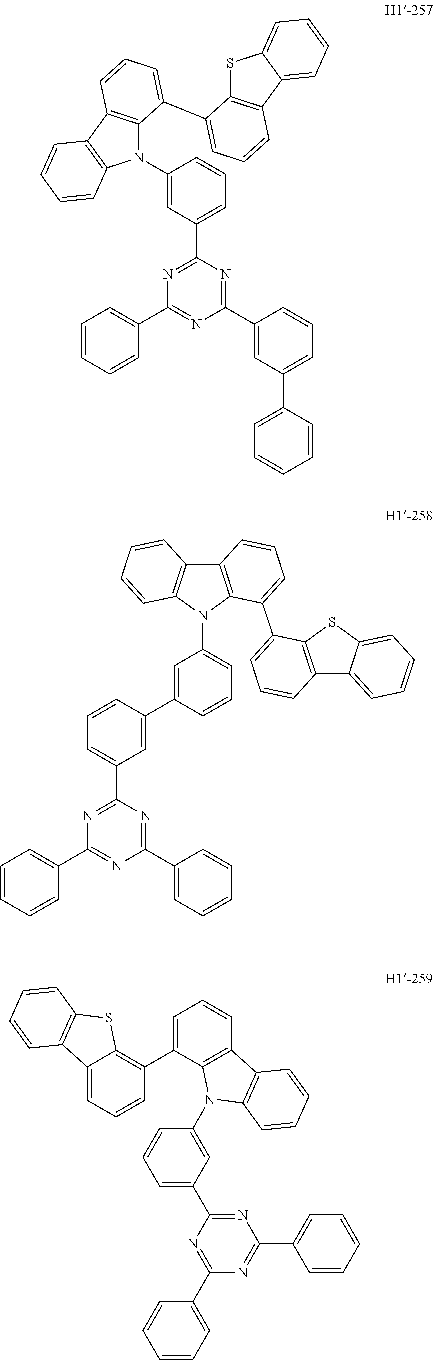 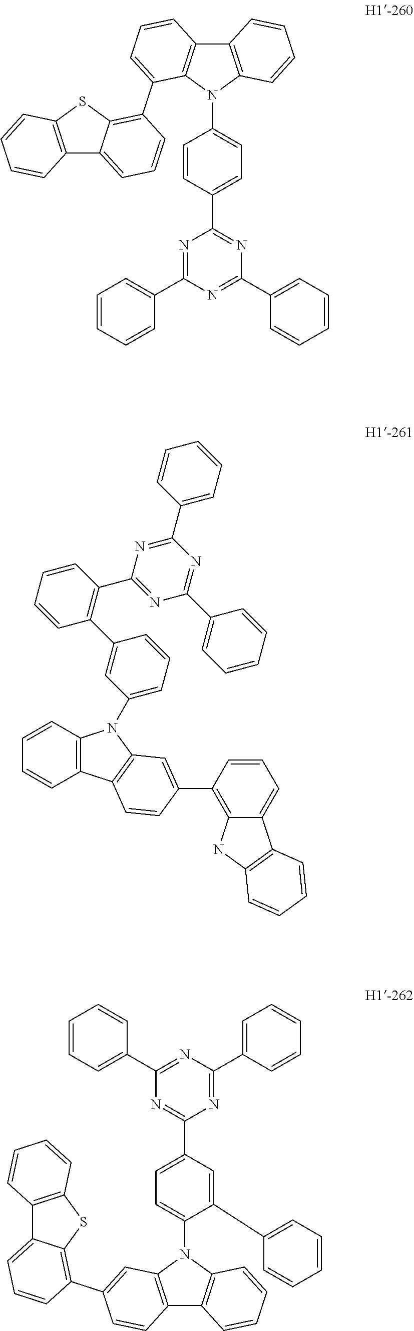 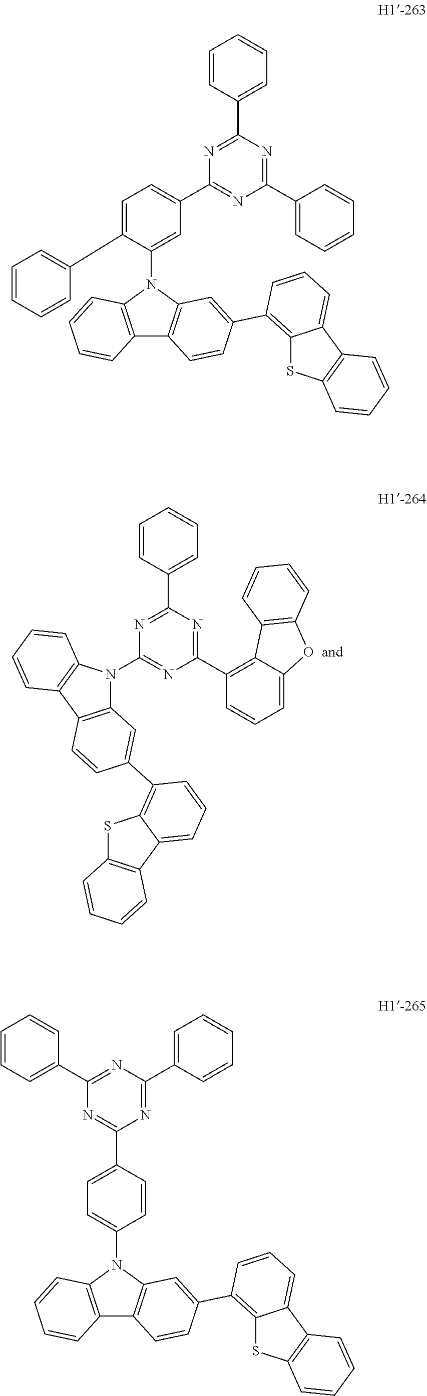
wherein, Dₙ represents that n number of hydrogens are replaced with deuterium; and n represents an integer of 5 to 50.

11. The plurality of host materials according to claim 1, wherein the compound represented by formula 2 is at least one selected from the following compounds: 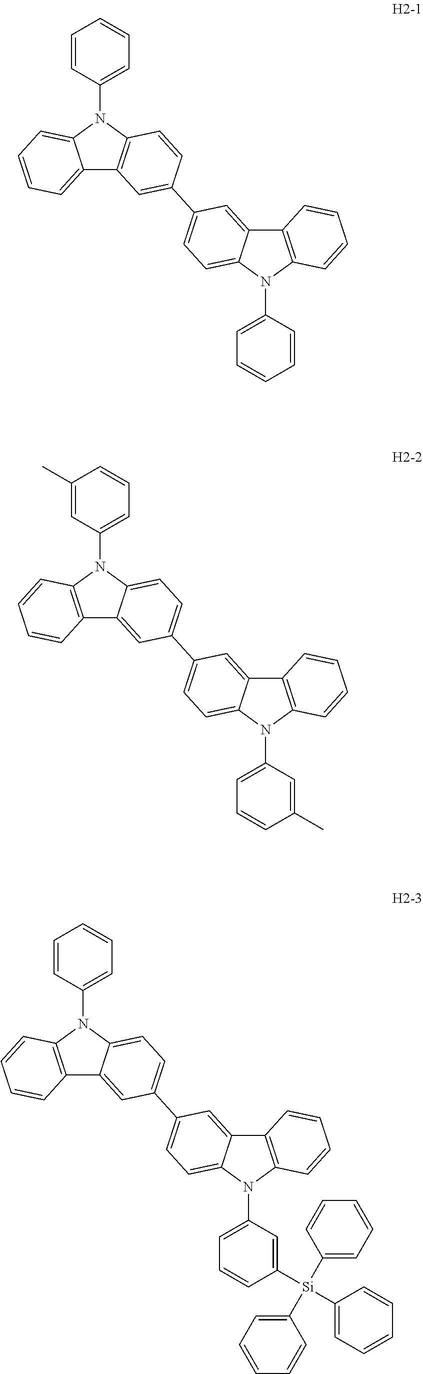 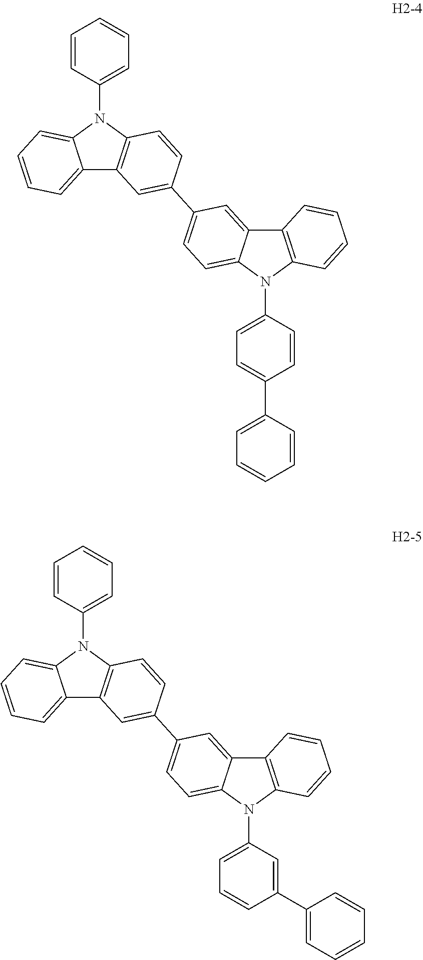 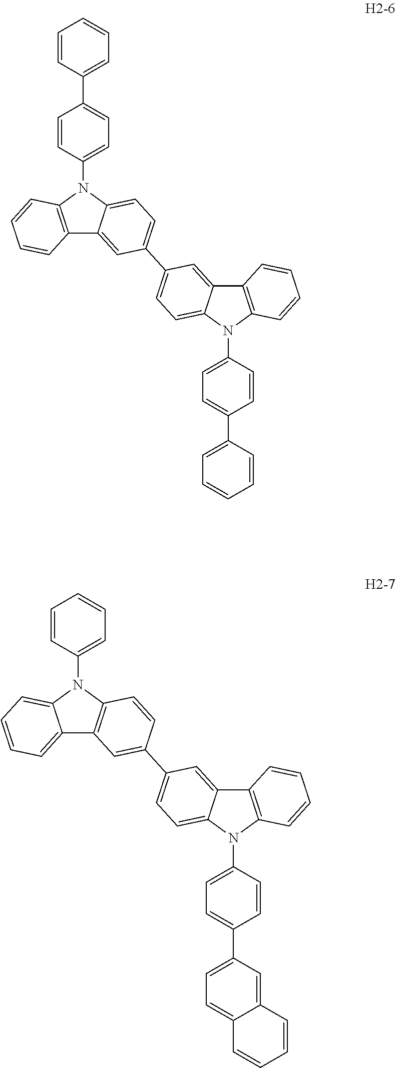 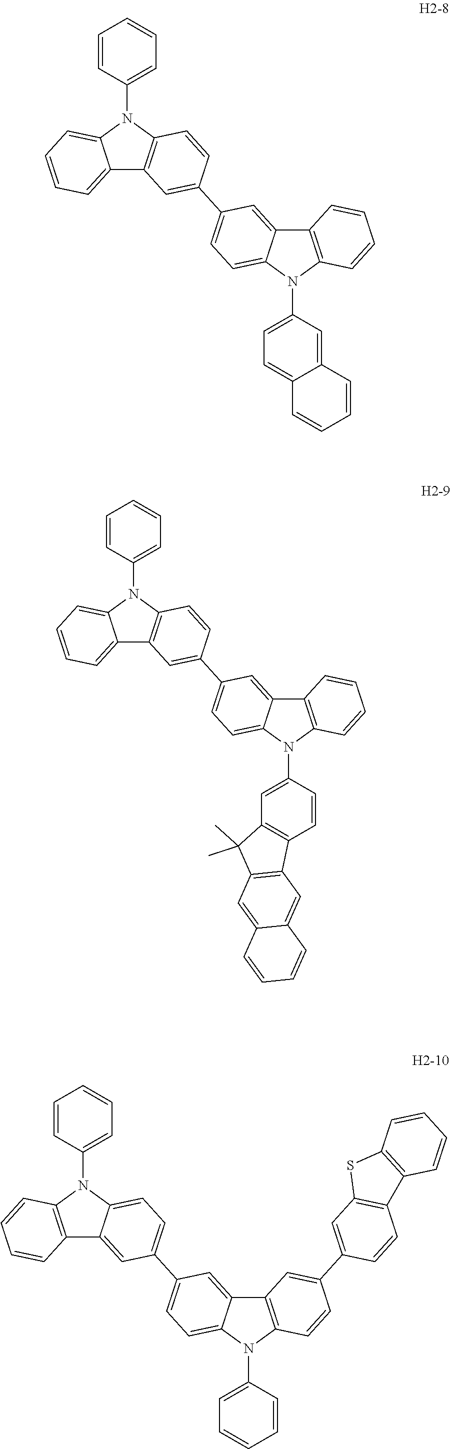 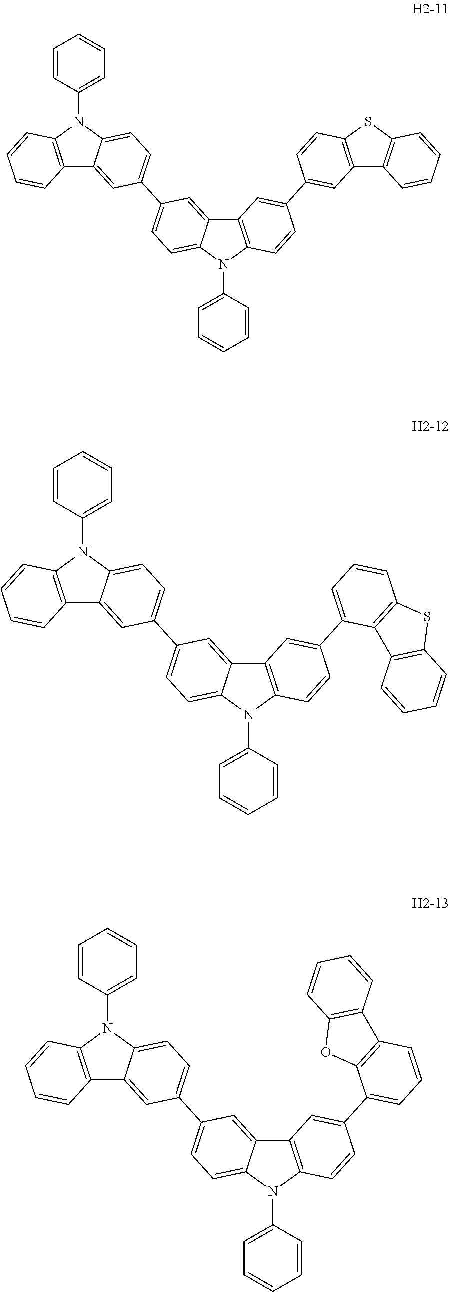 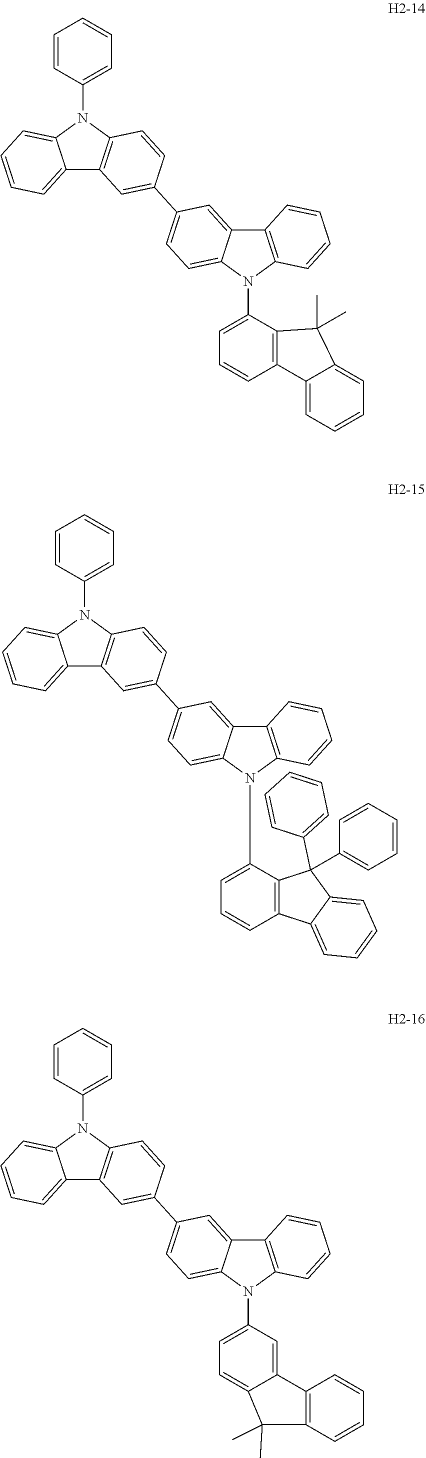 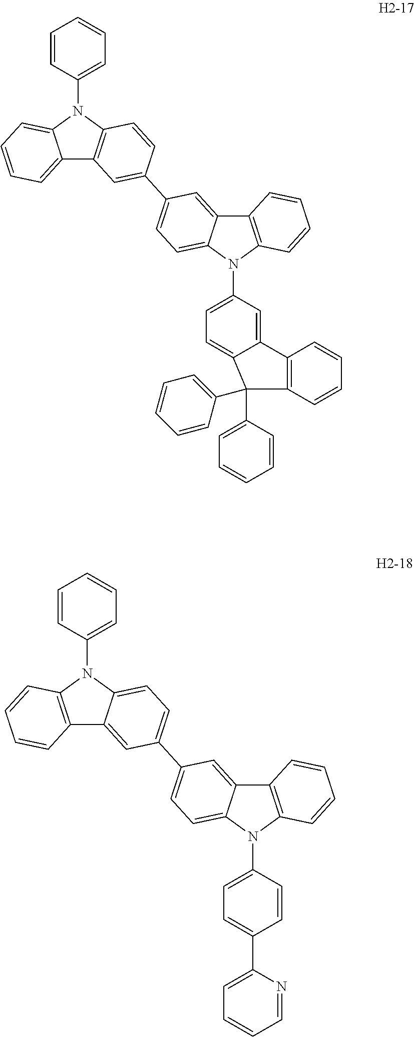 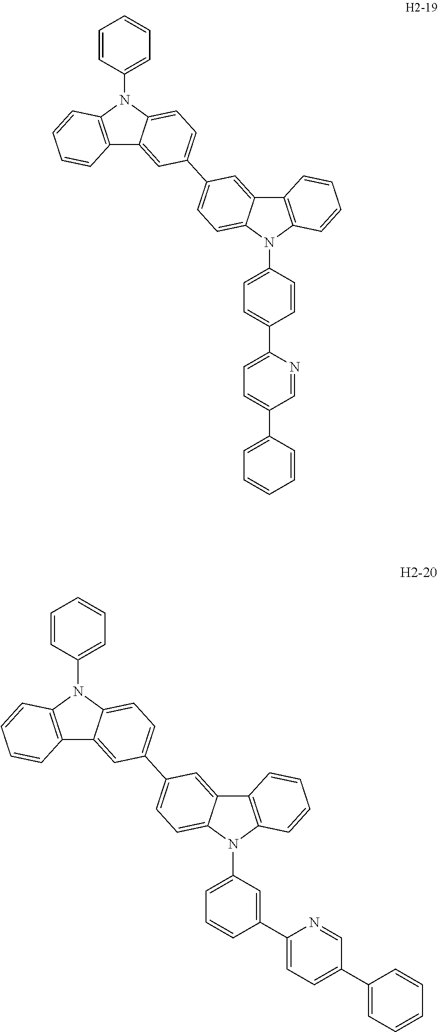 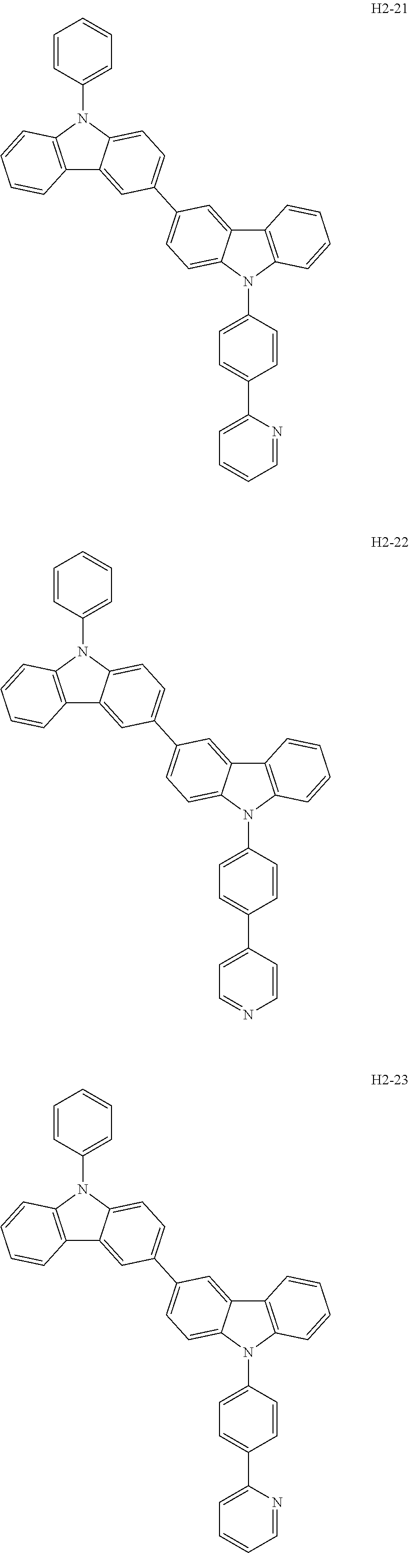 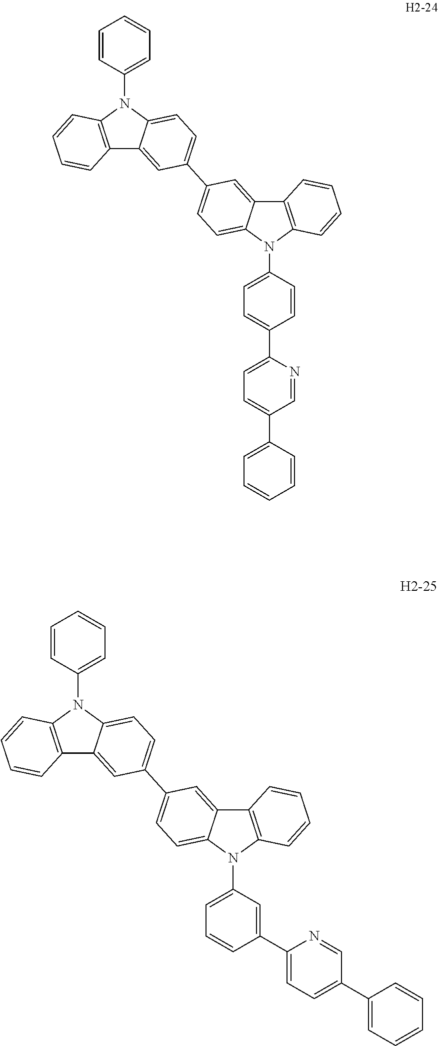 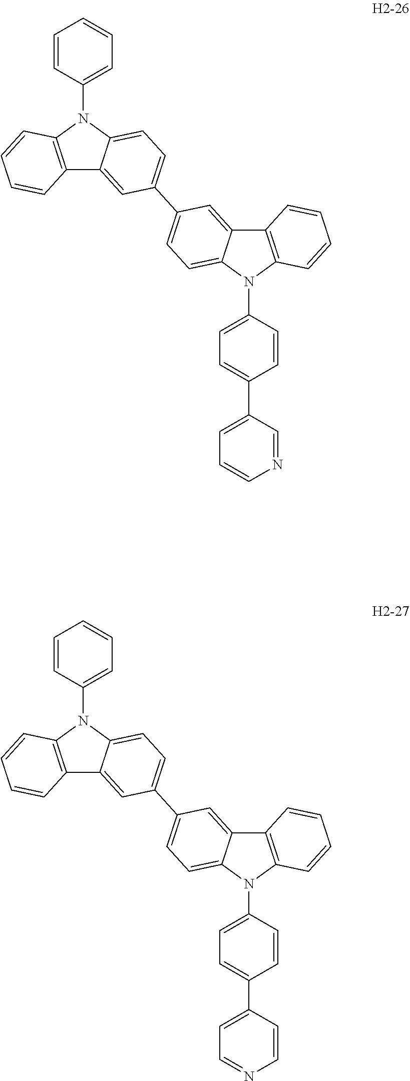 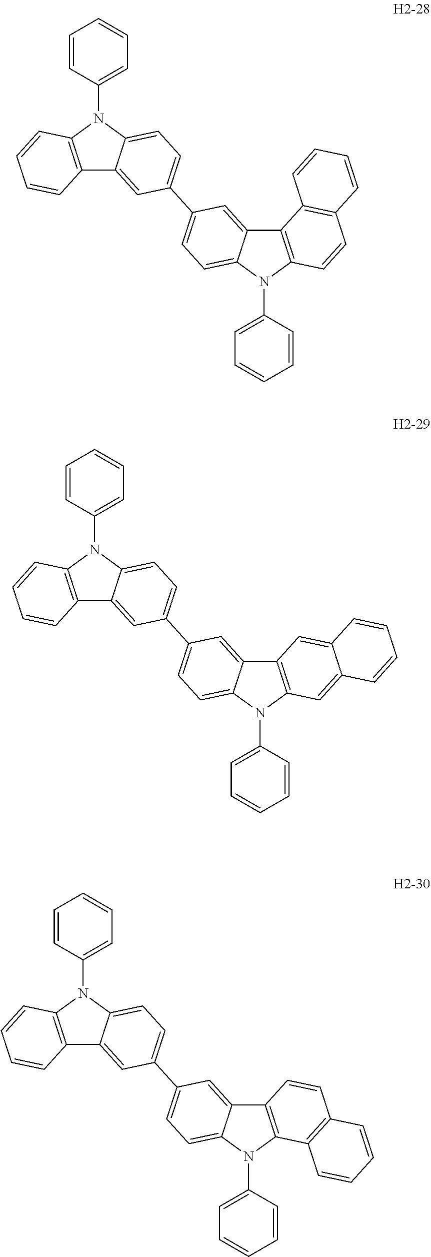 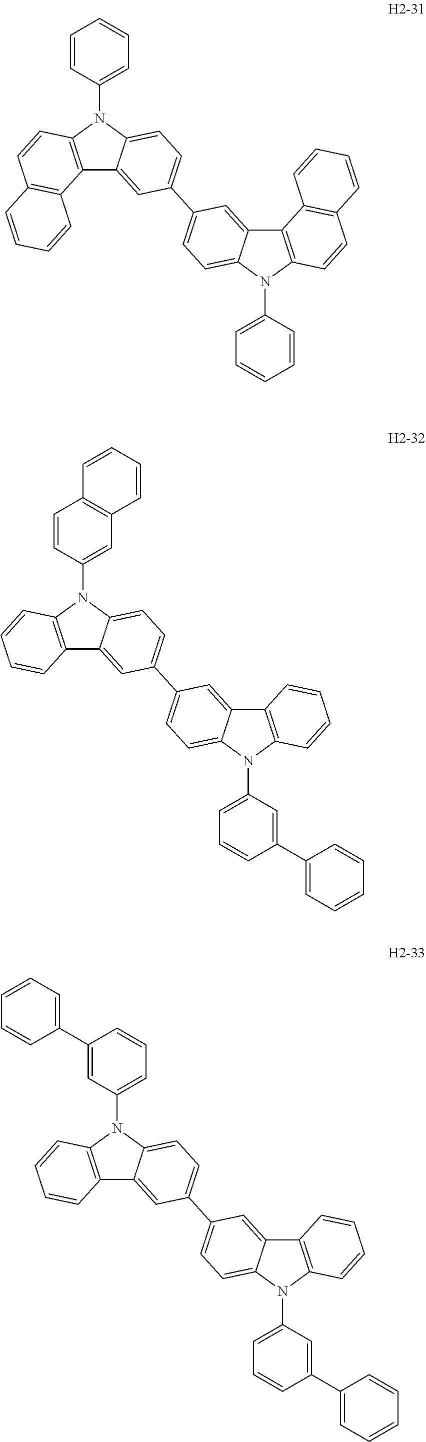 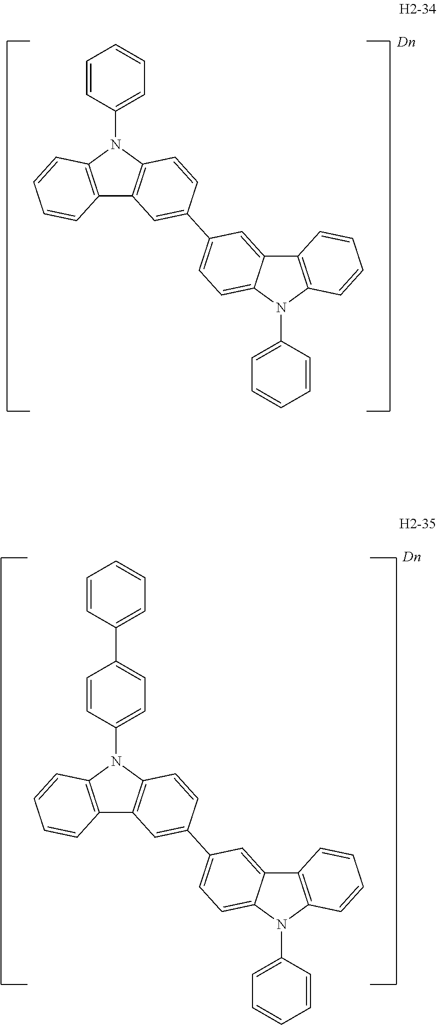 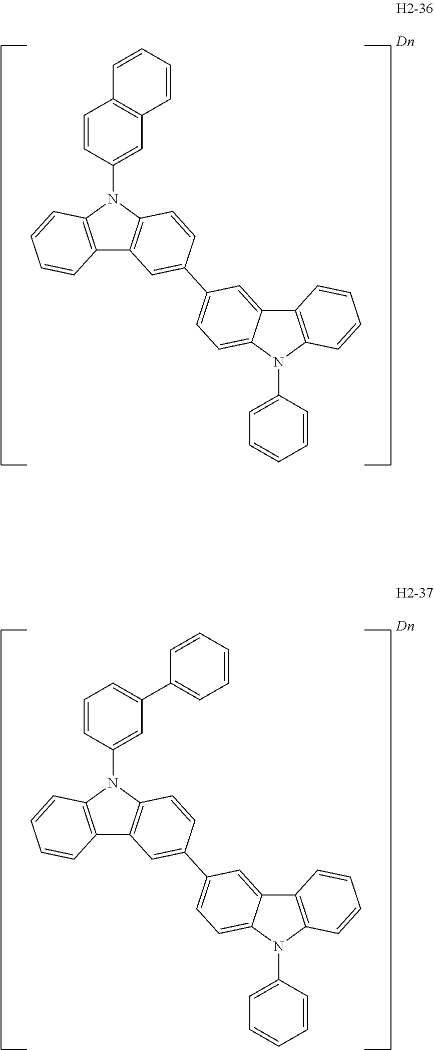 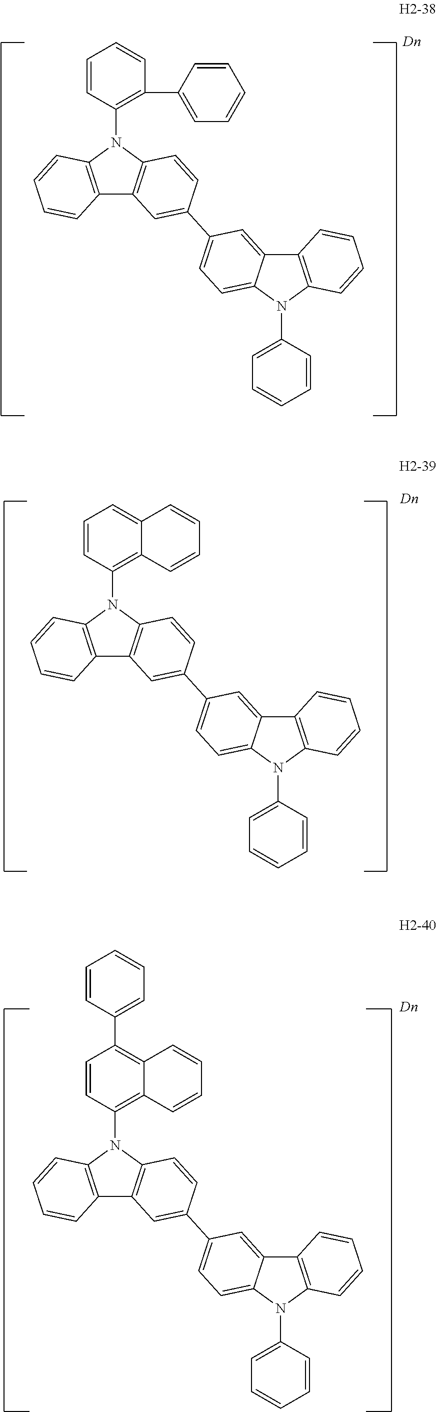 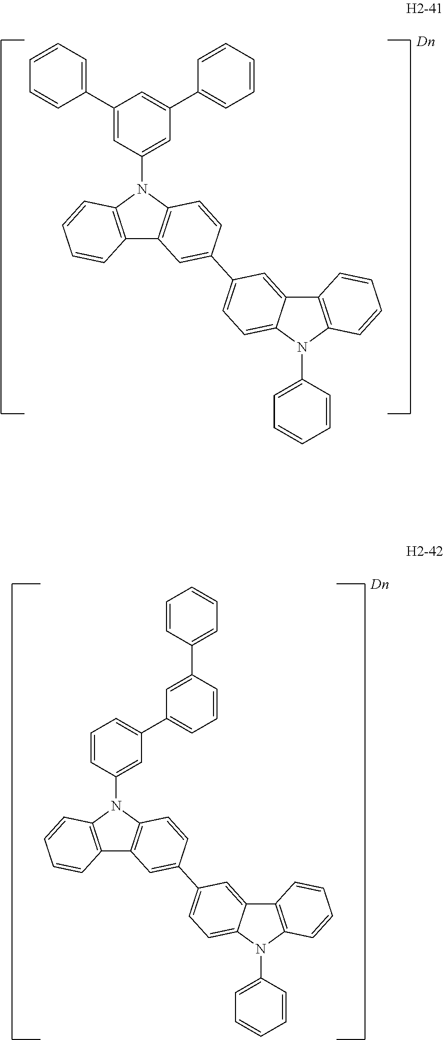 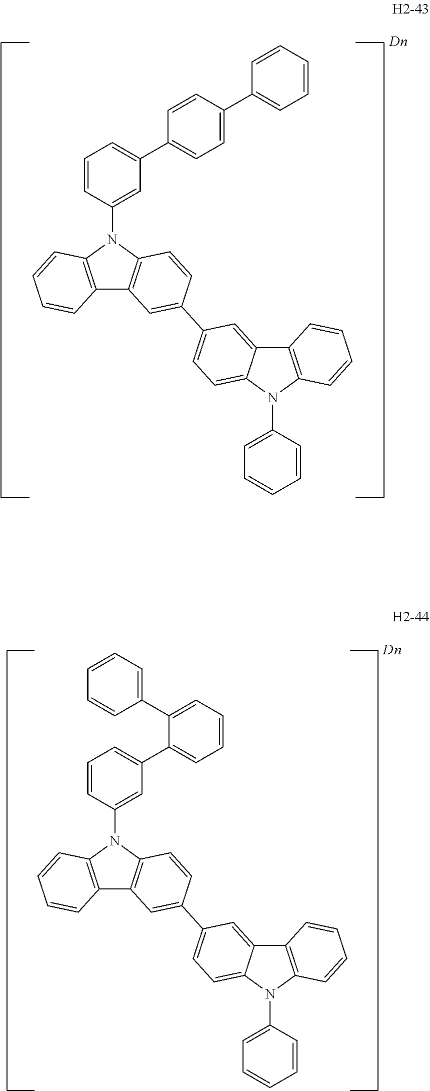 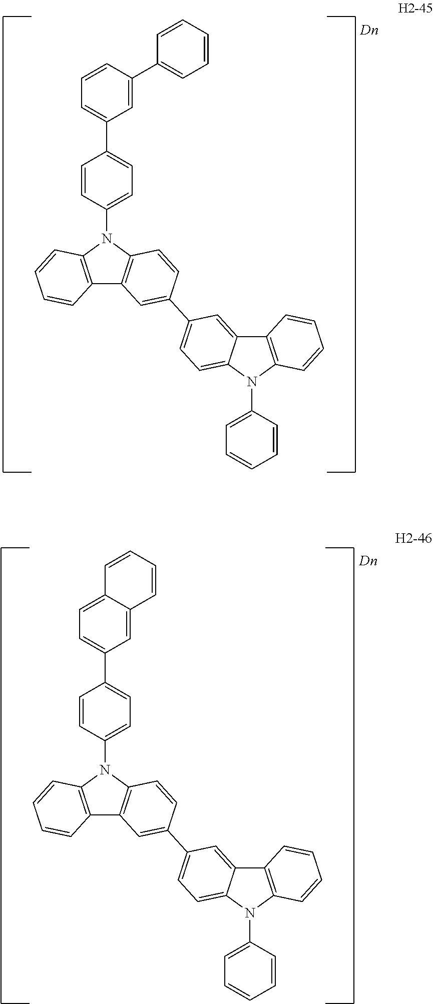 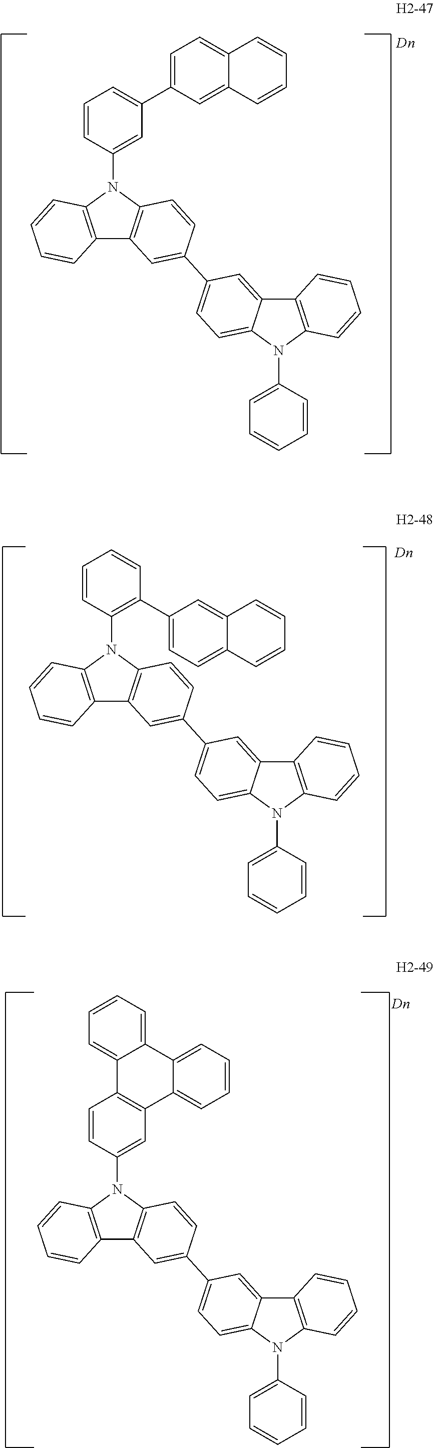 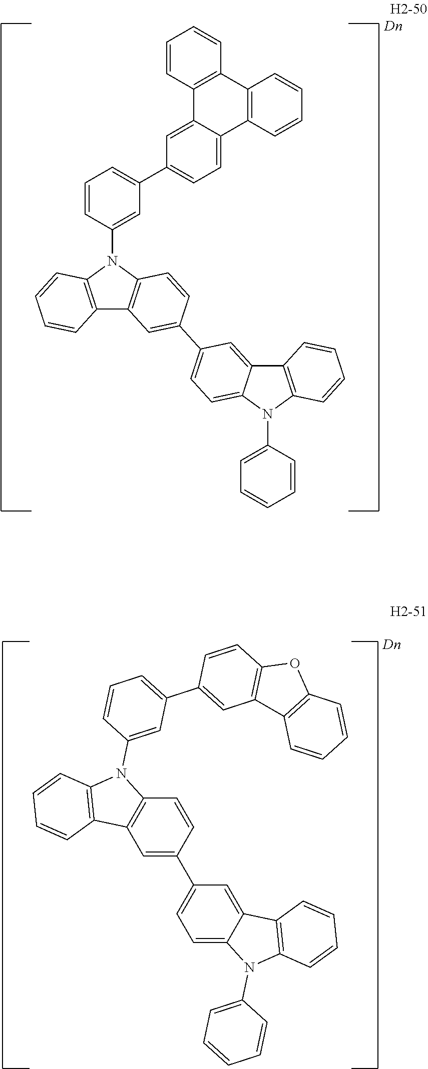 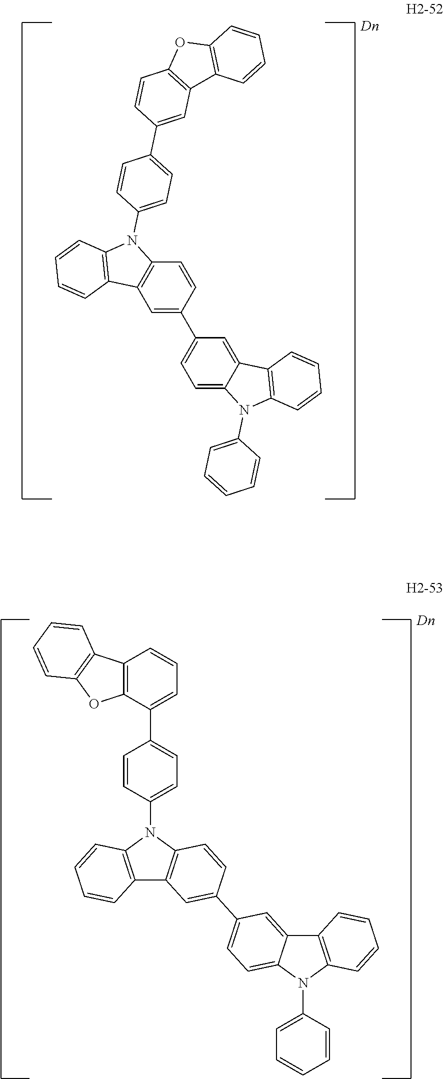 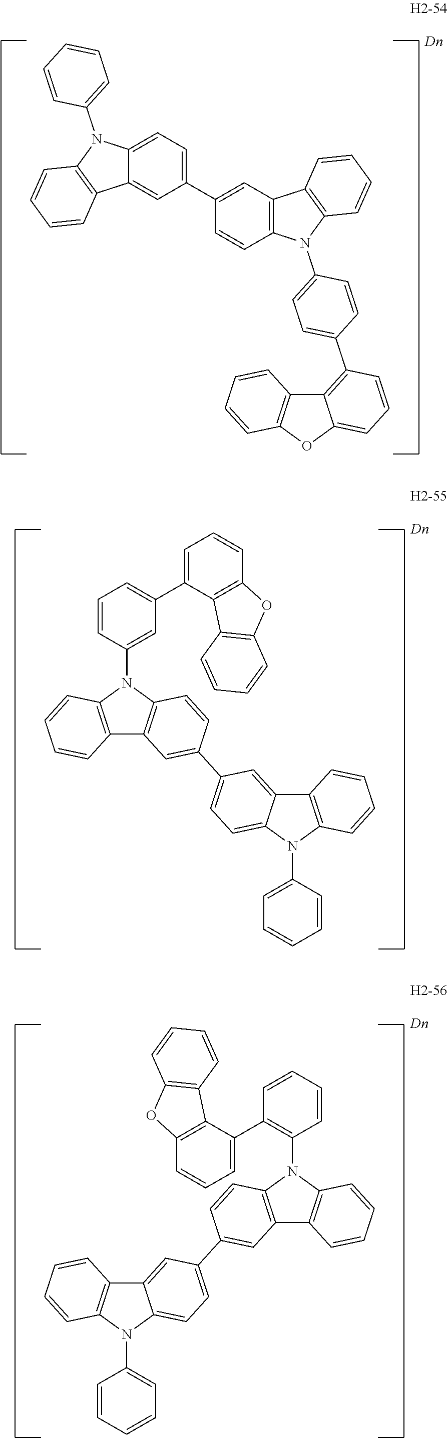 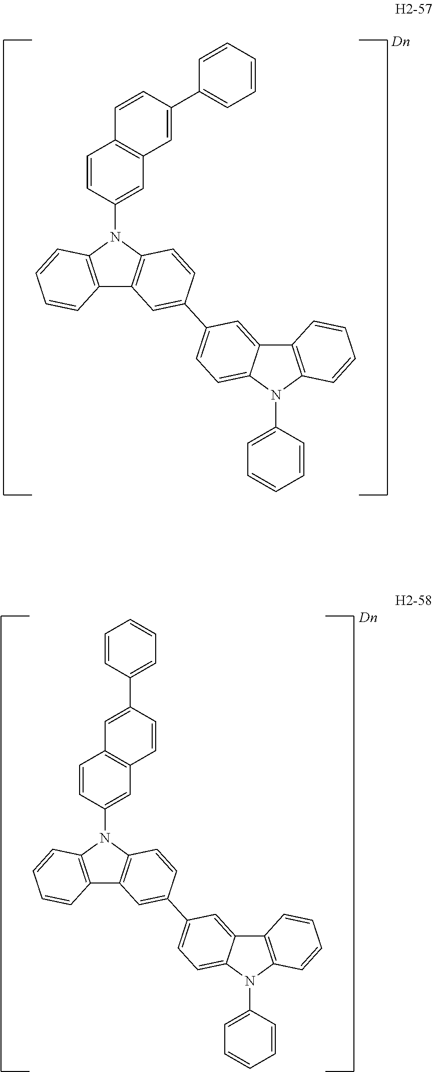 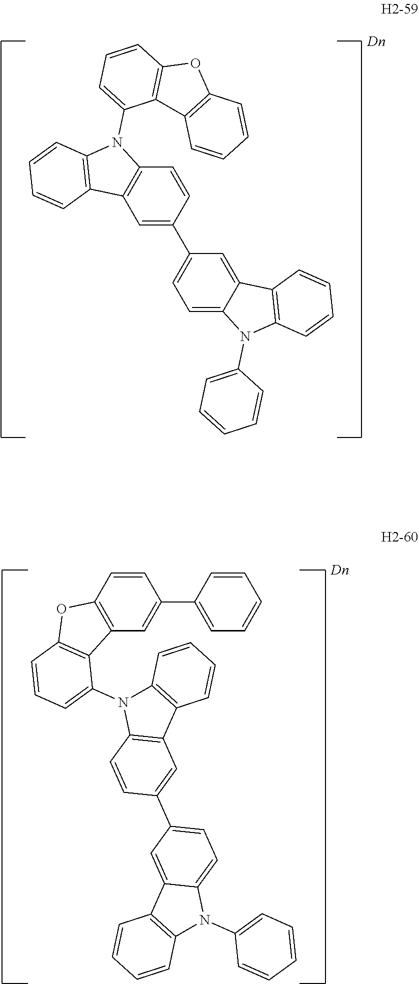 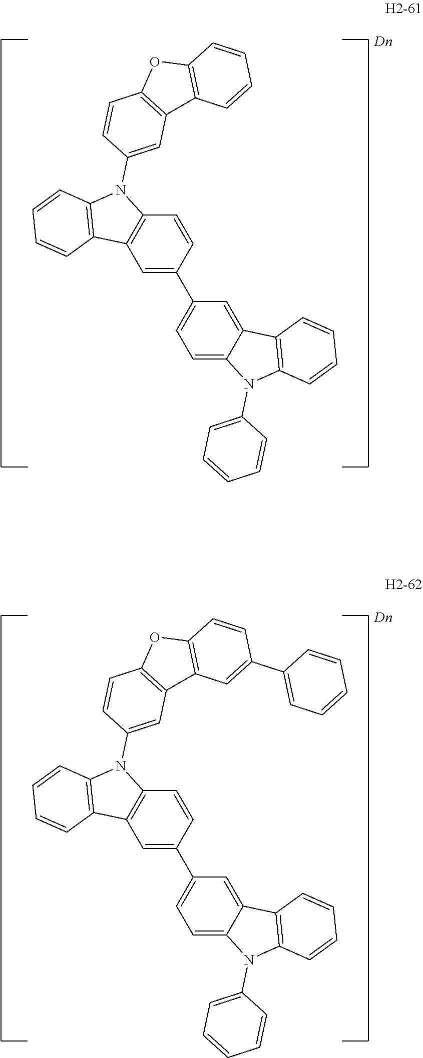 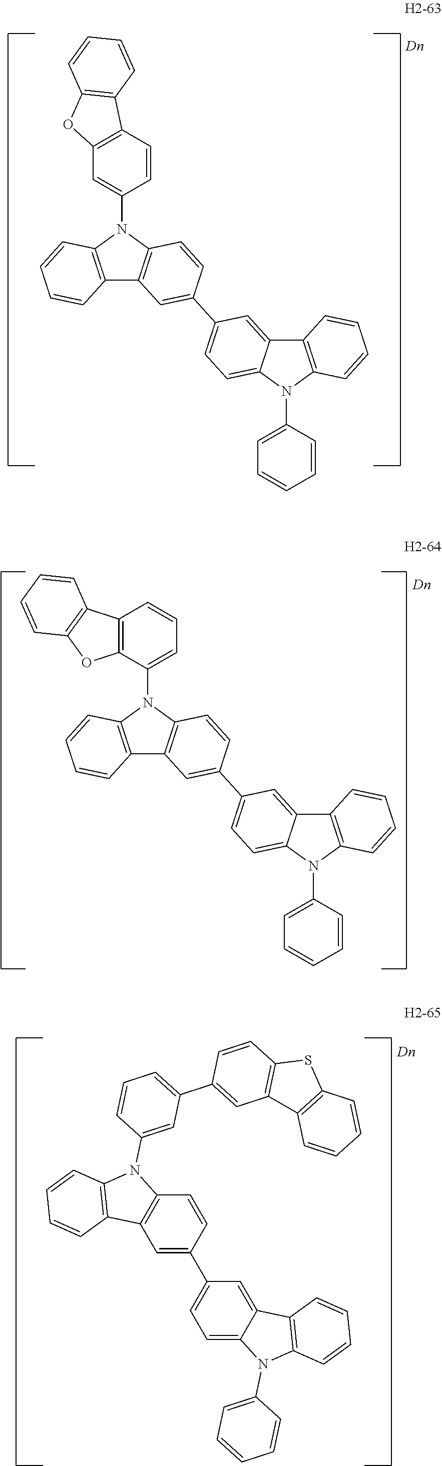 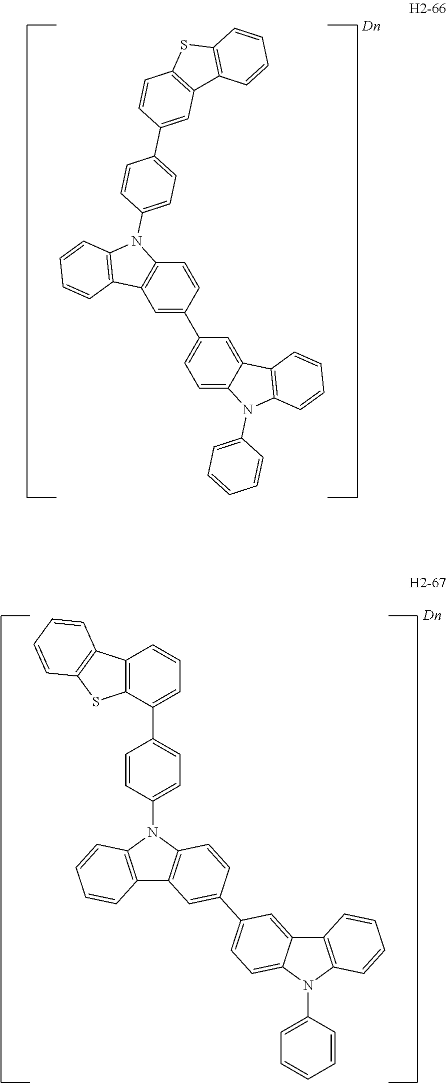 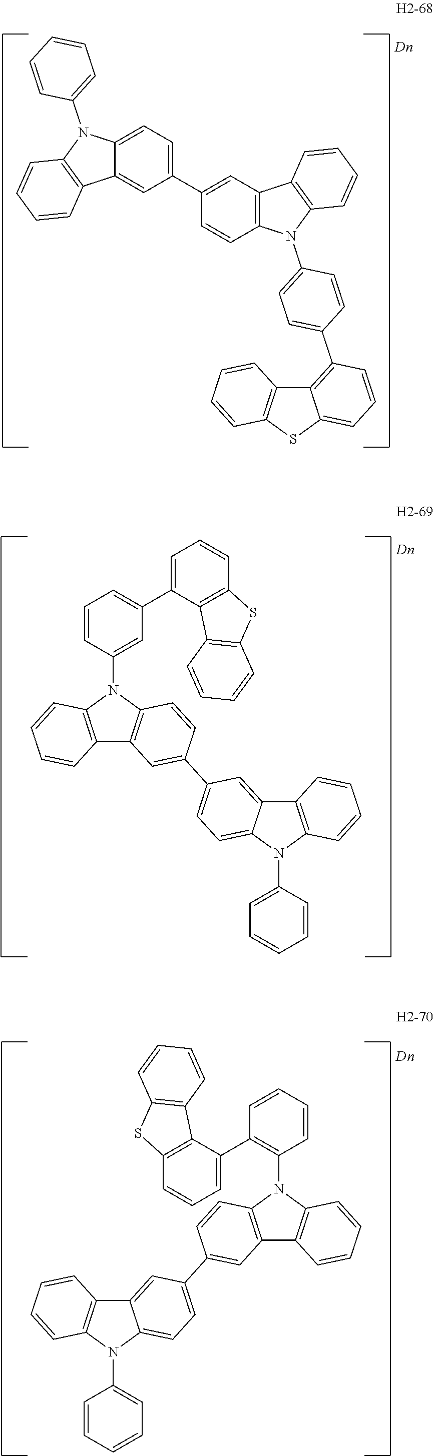 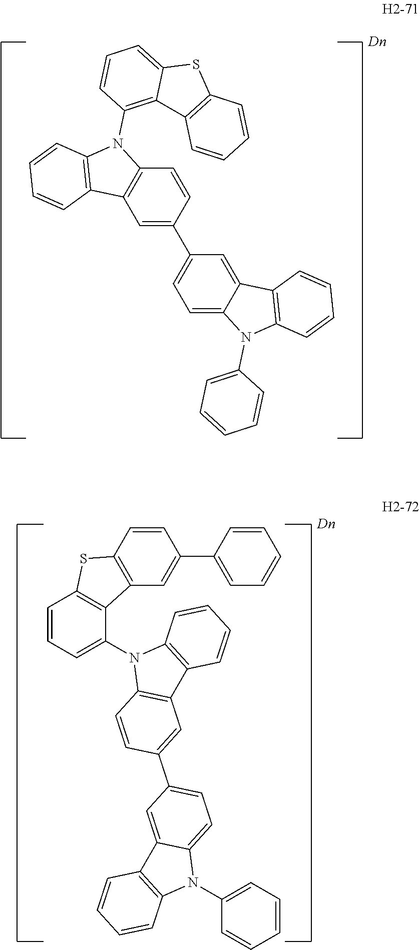 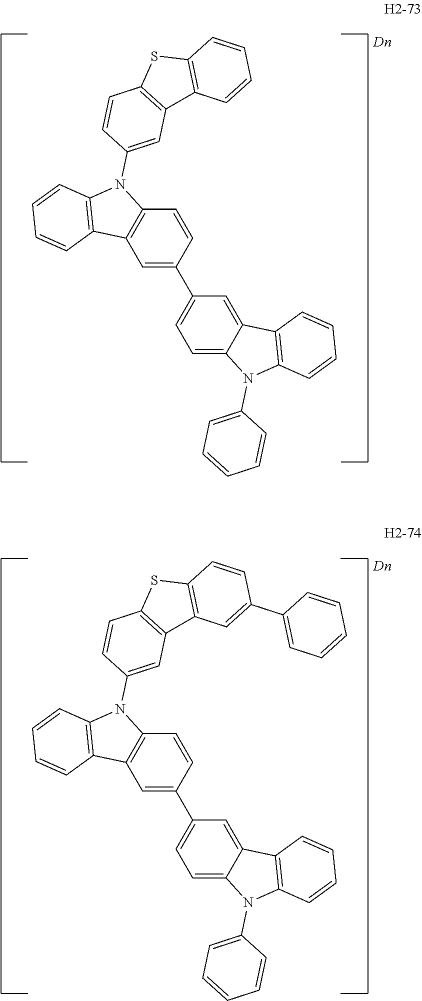 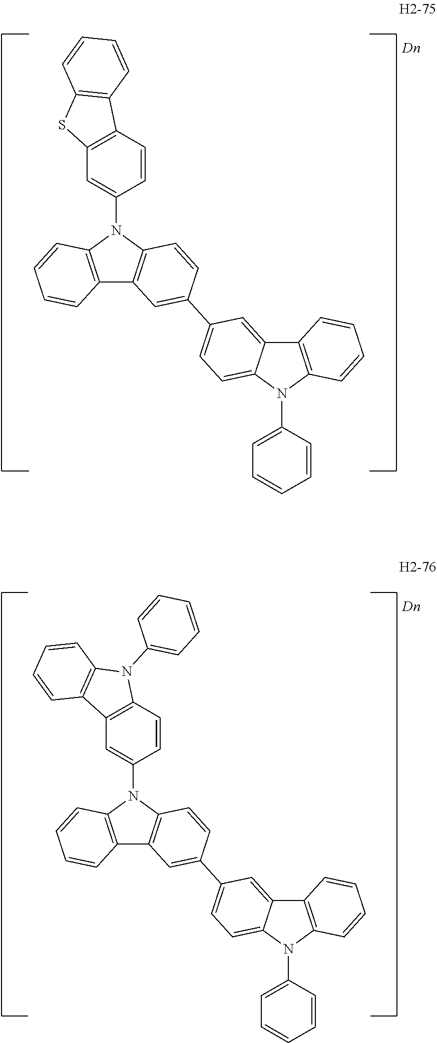 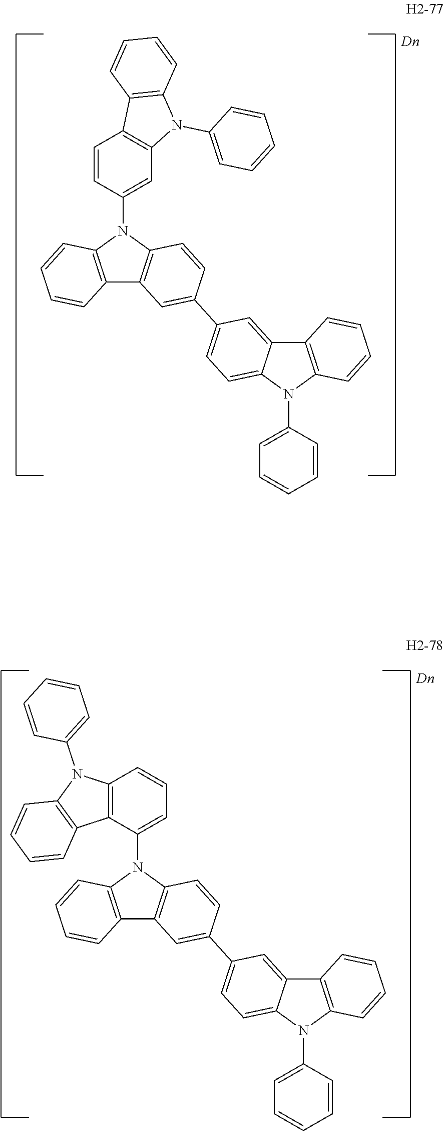 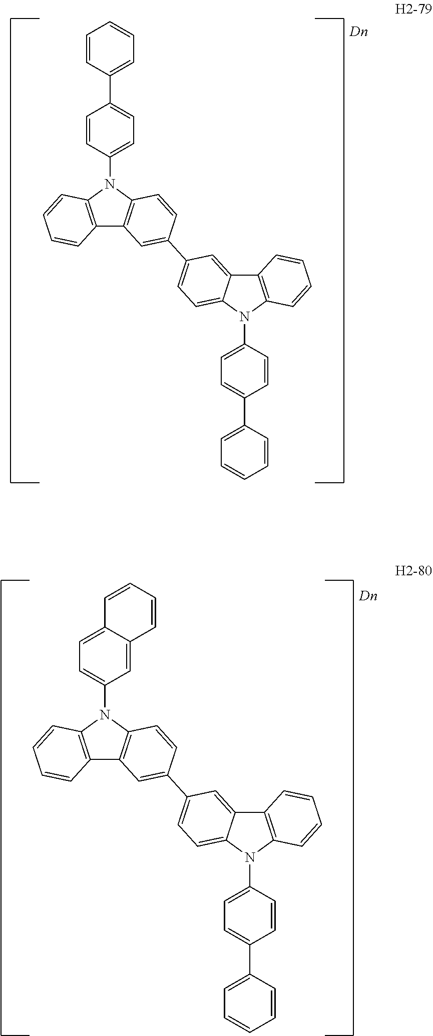 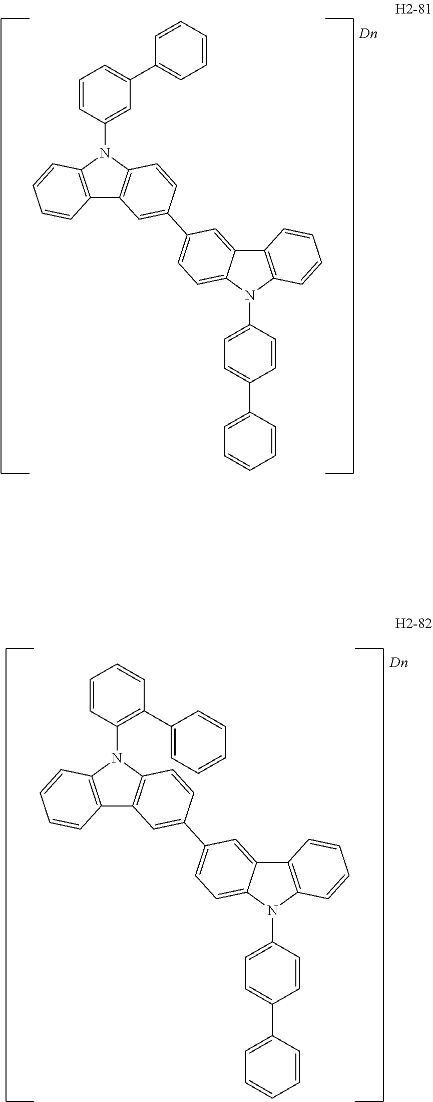 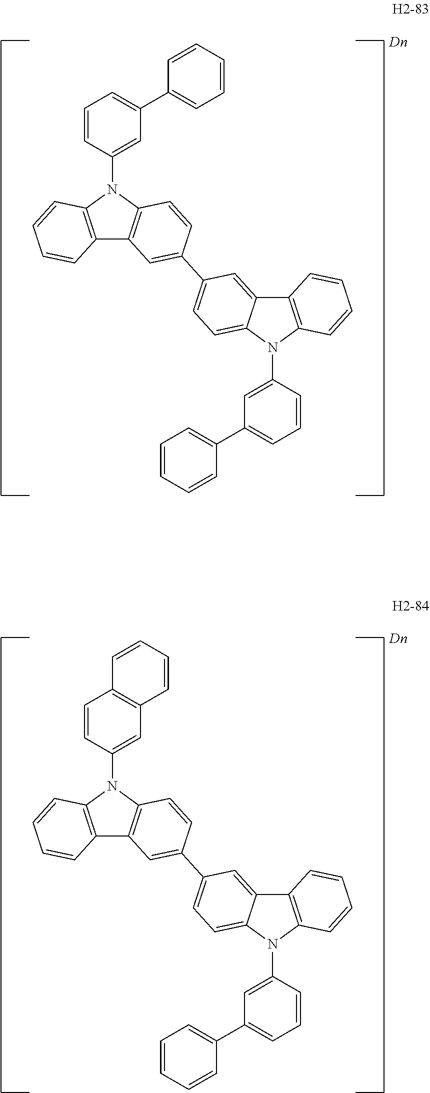 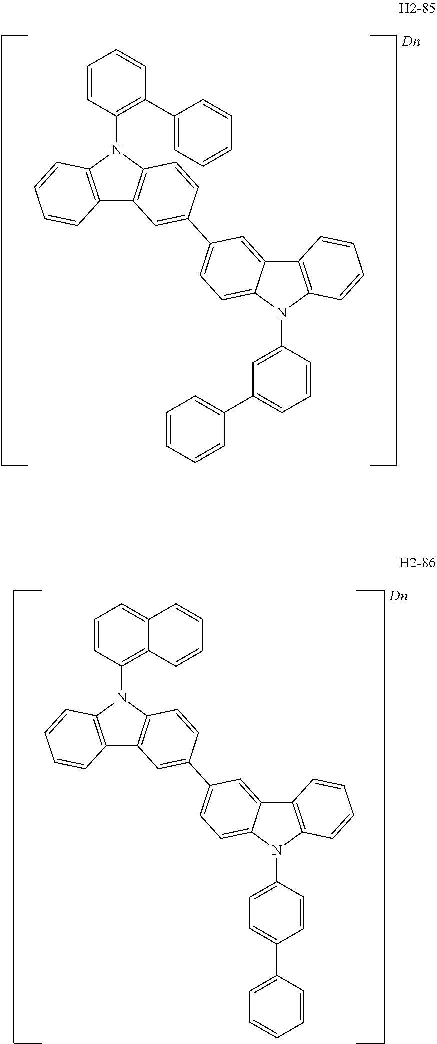 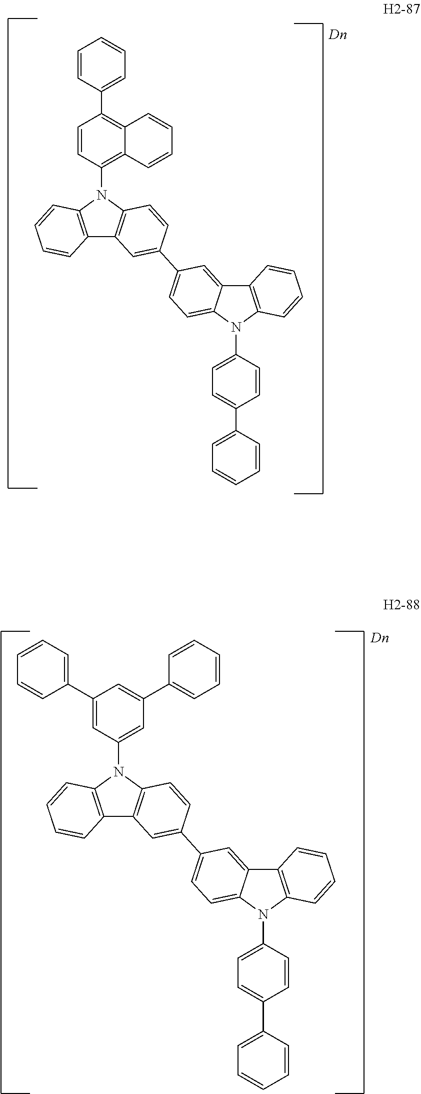 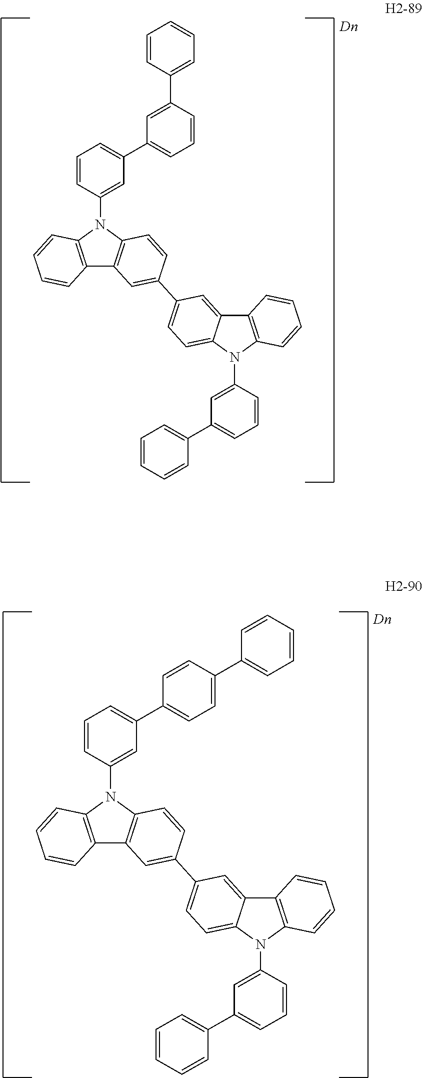 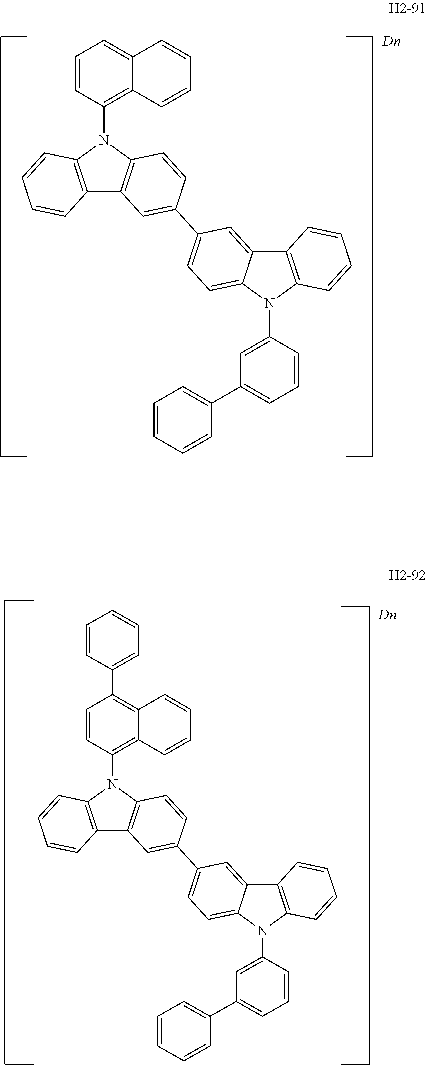 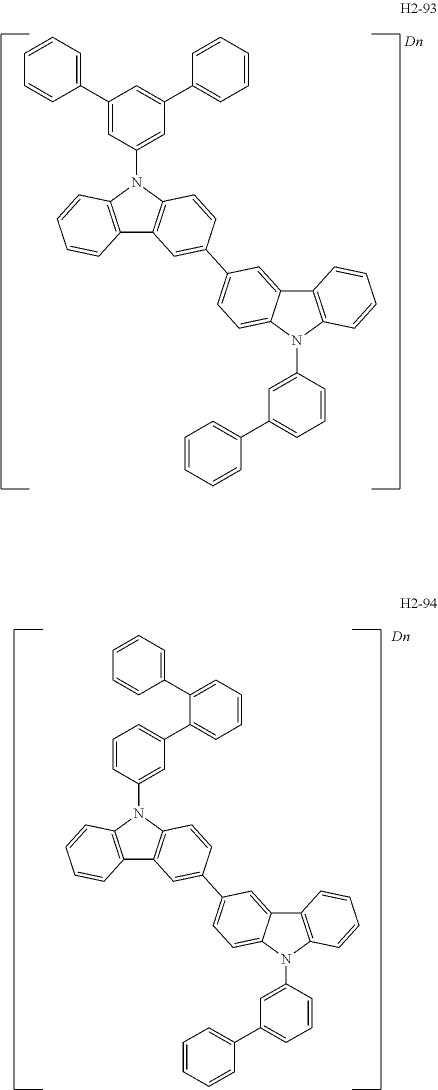 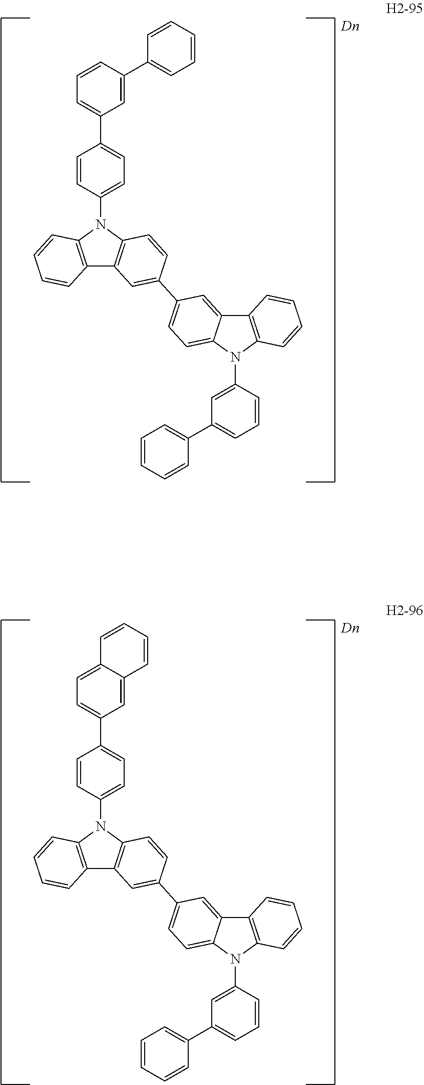 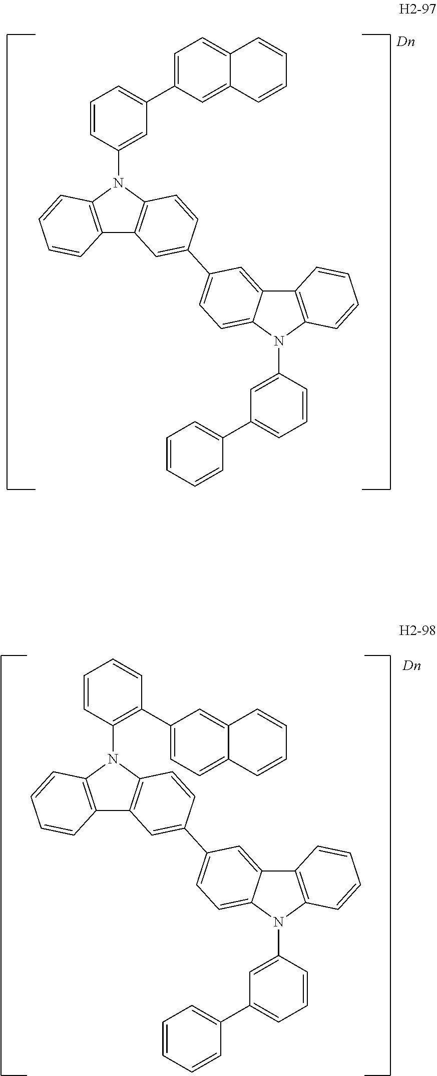 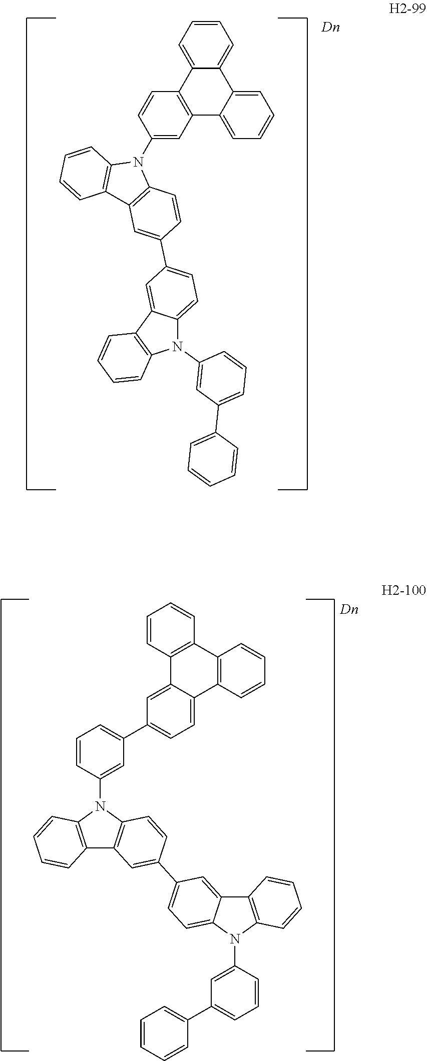 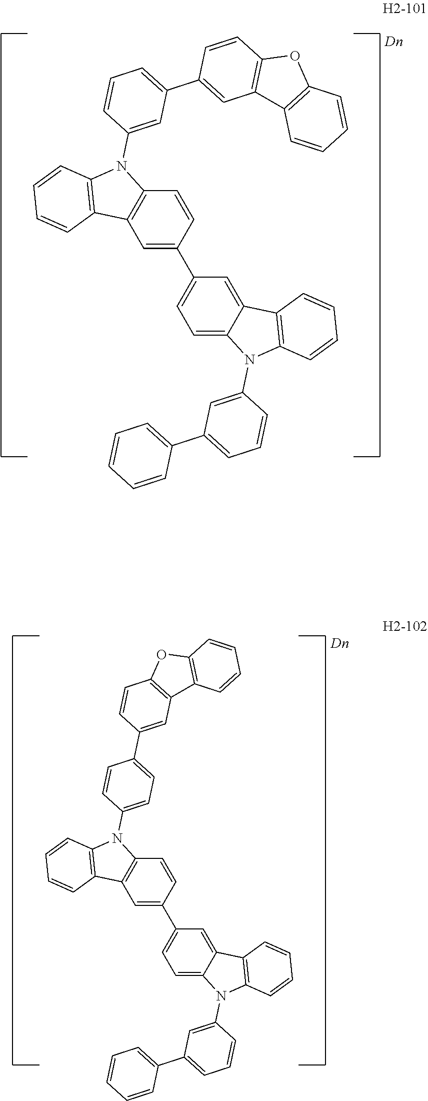 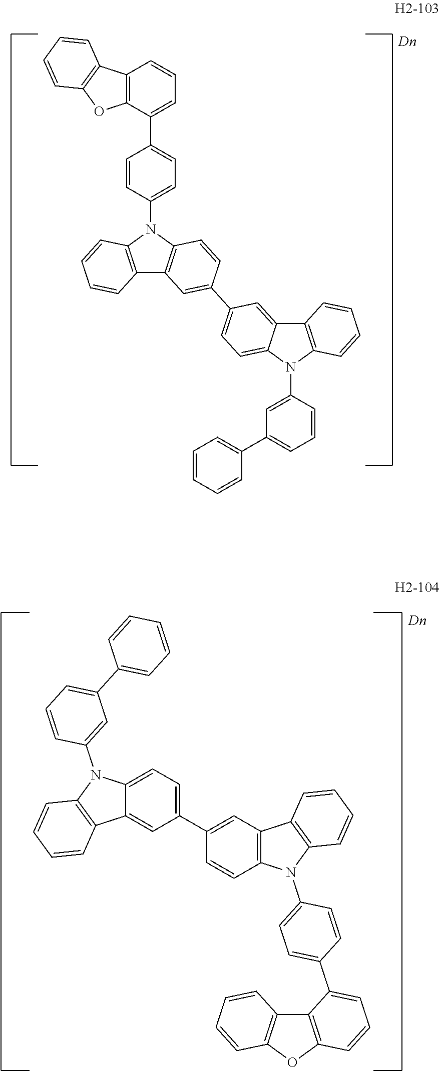 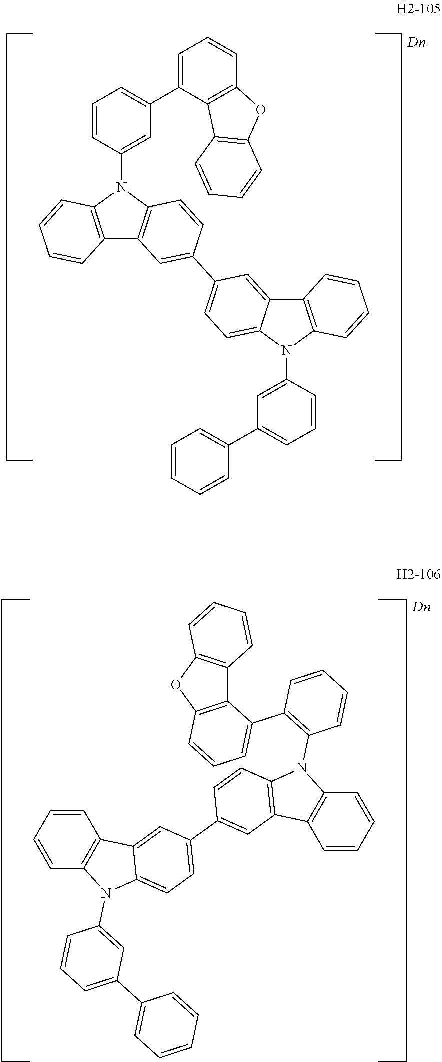 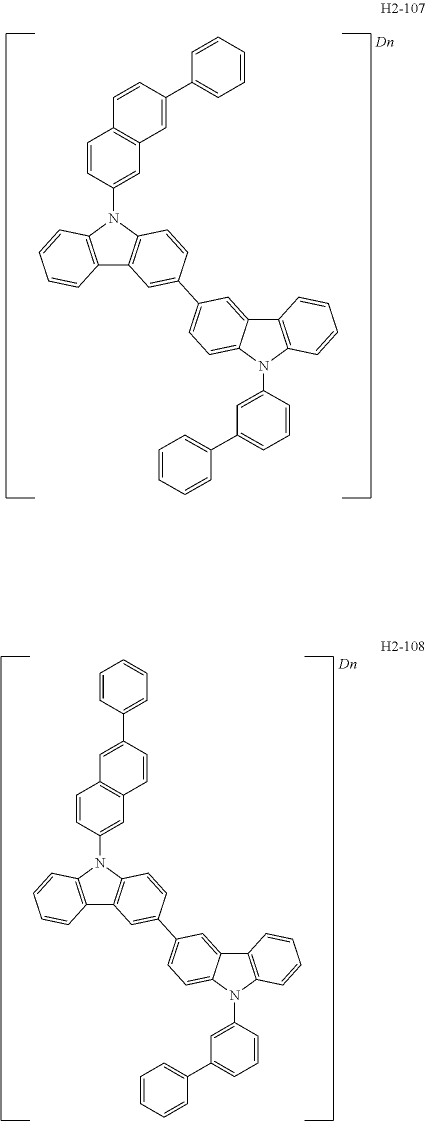 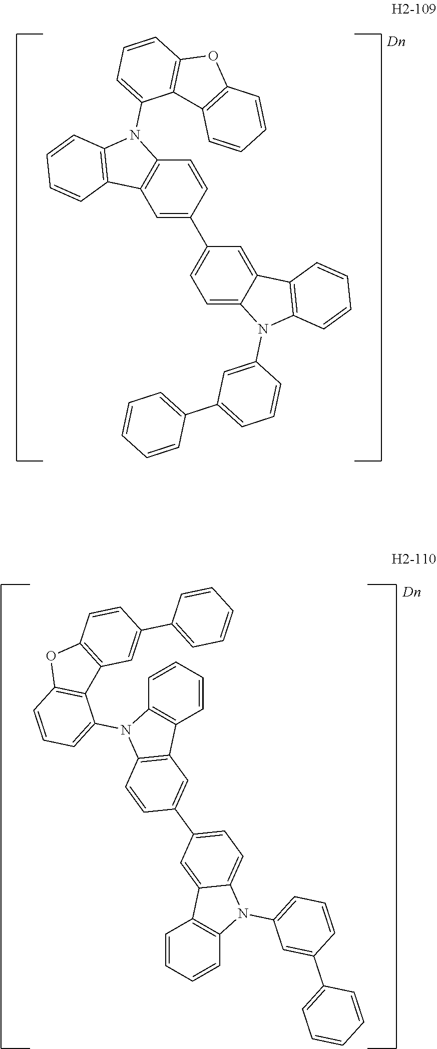 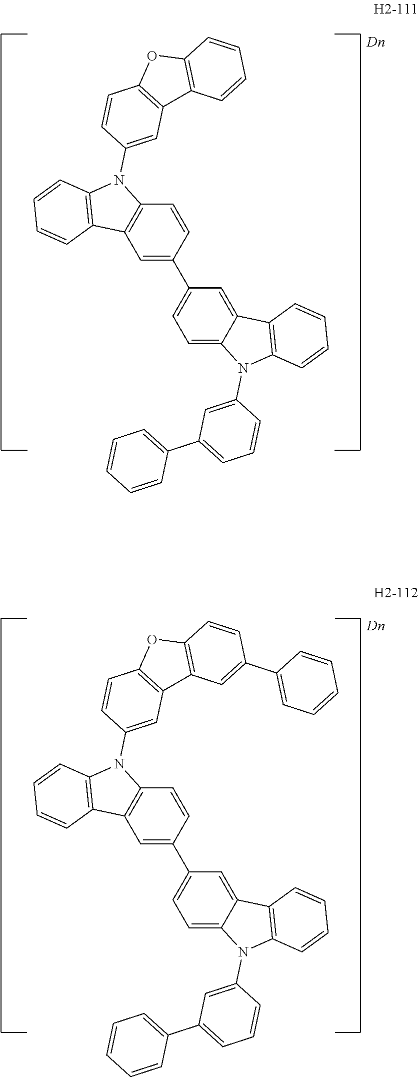 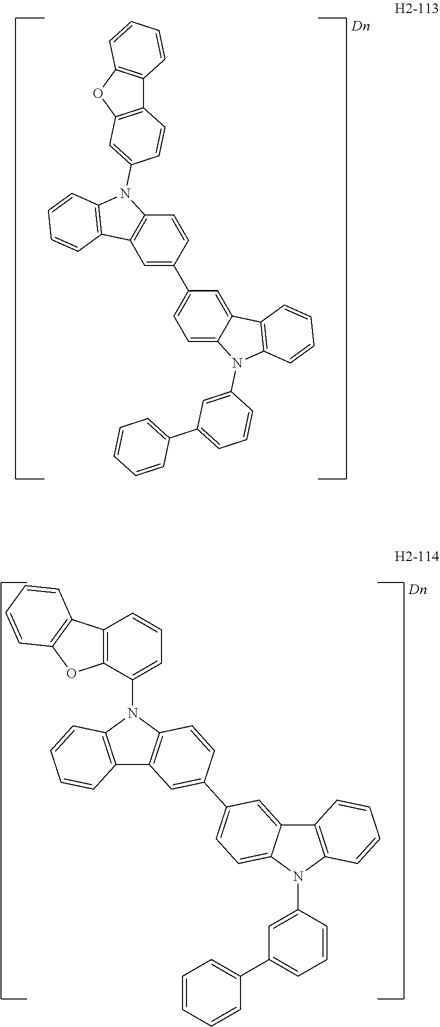 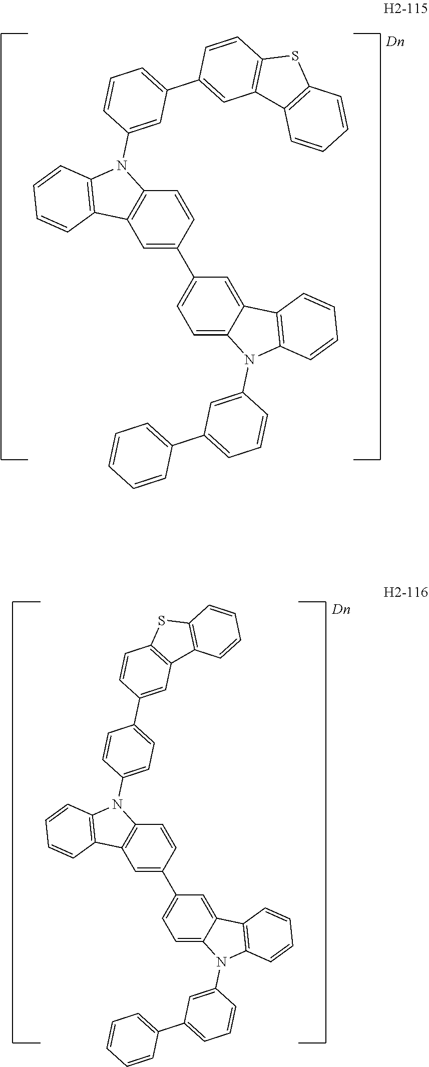 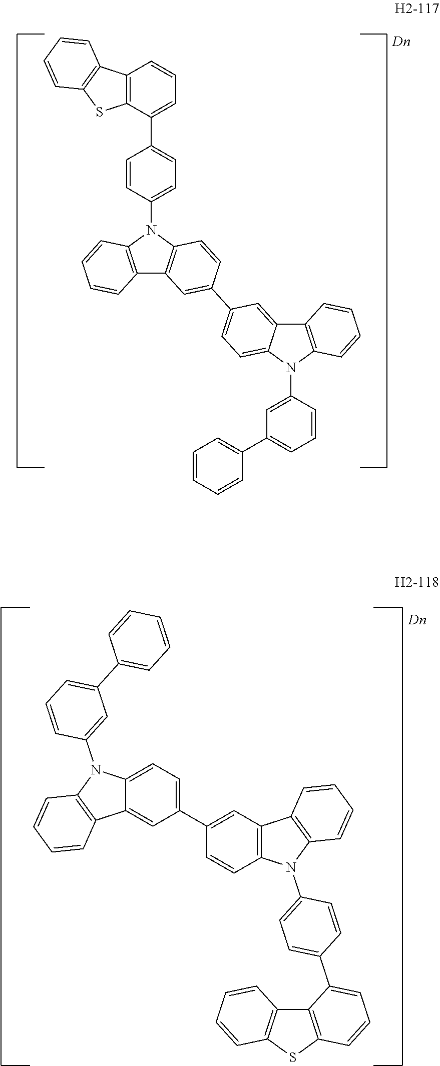 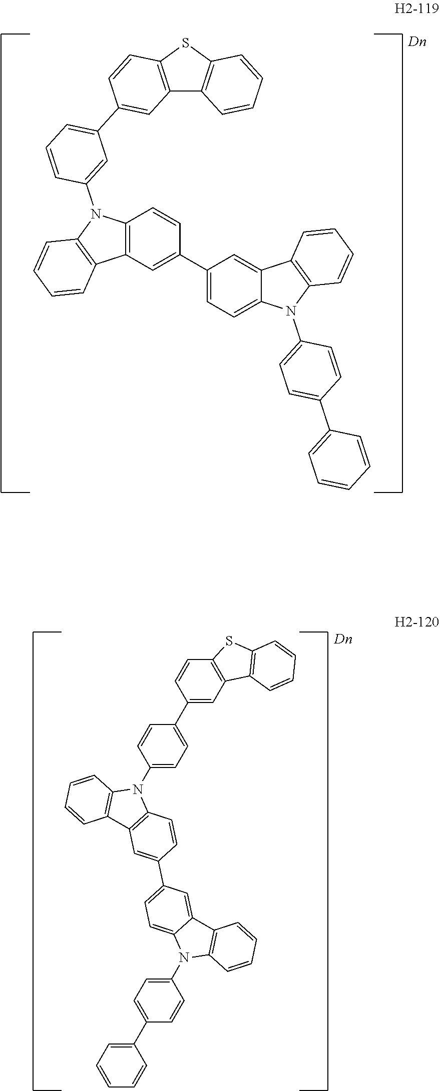 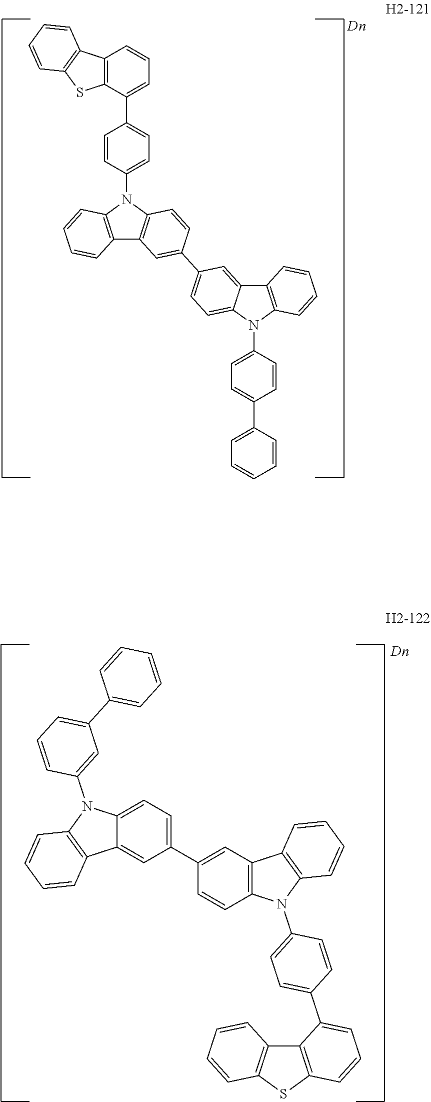 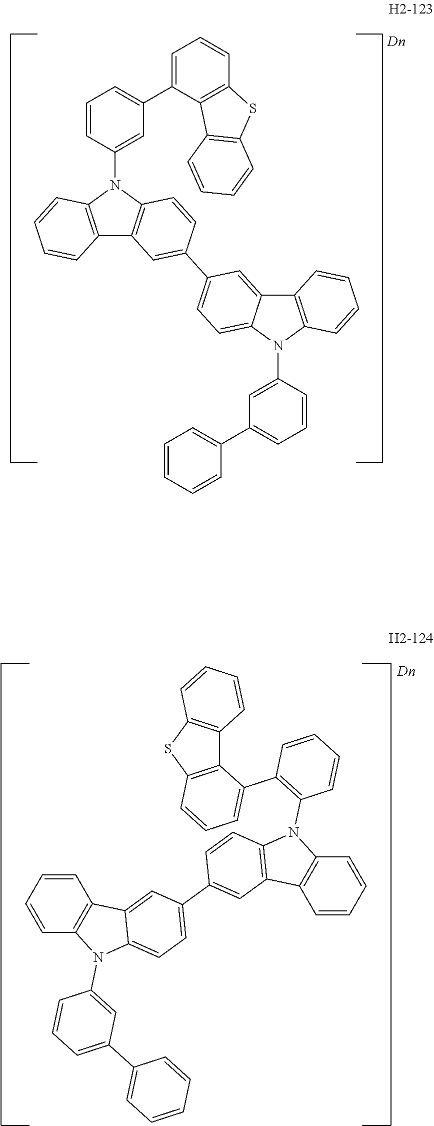 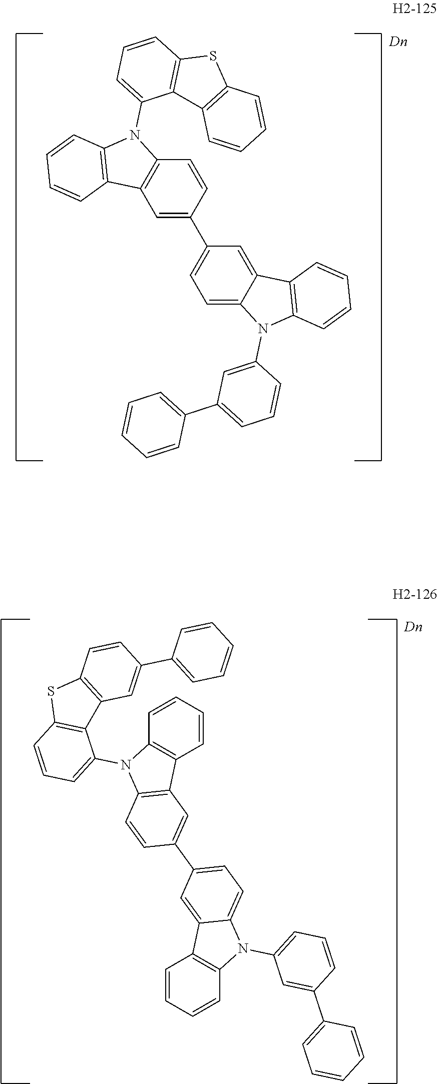 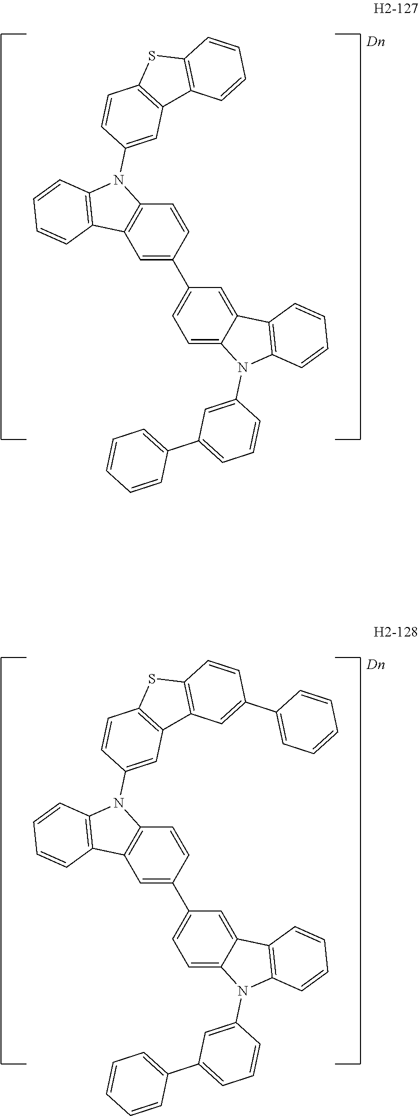 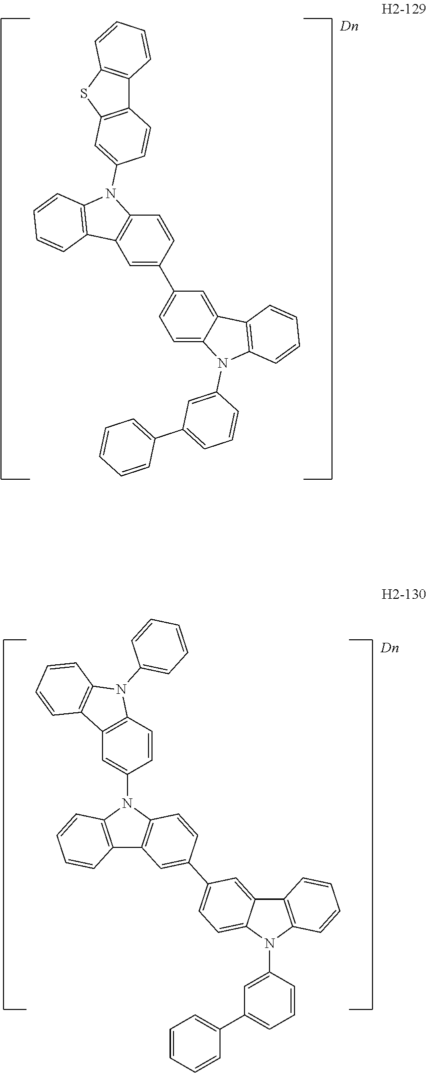 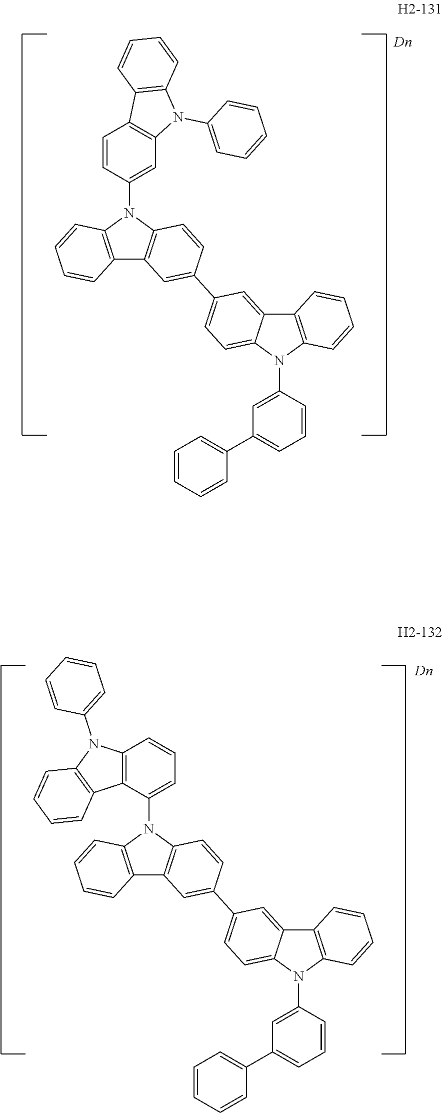 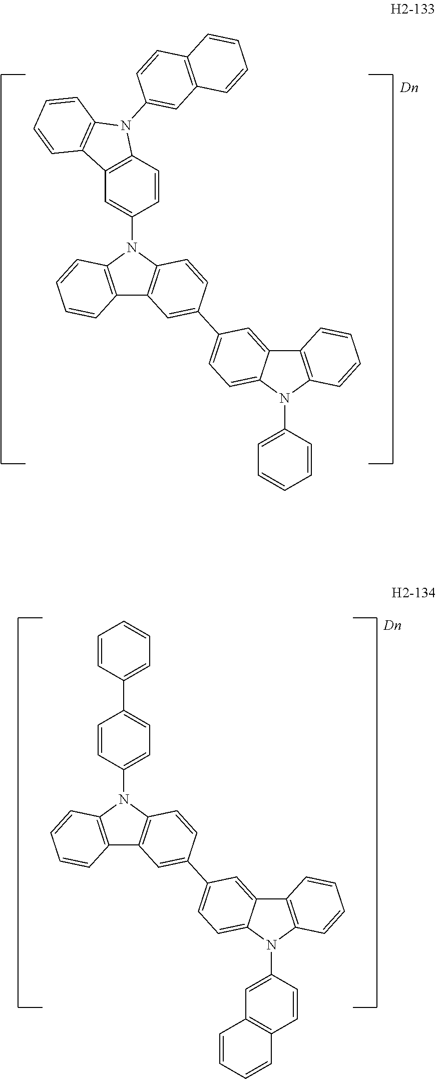 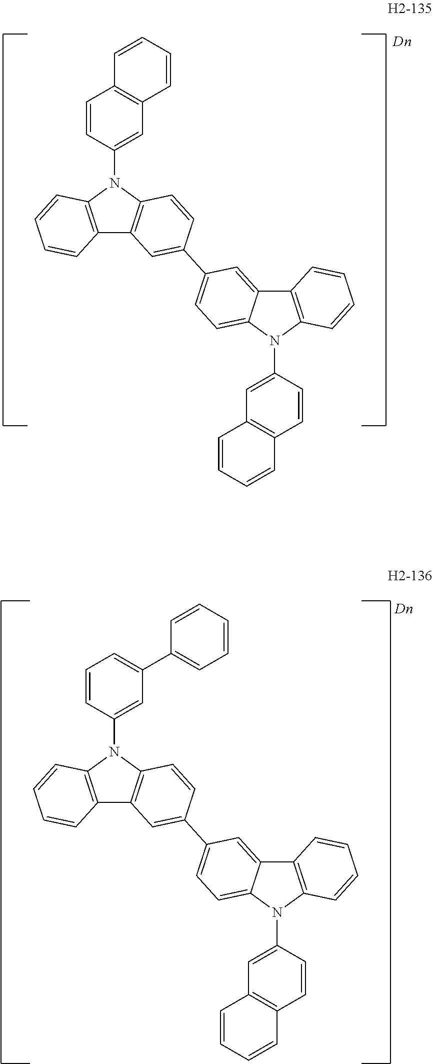 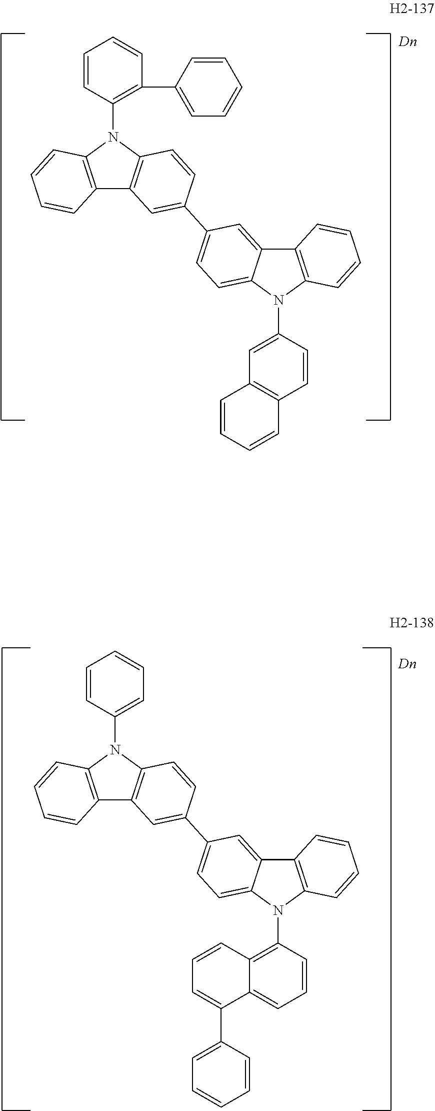 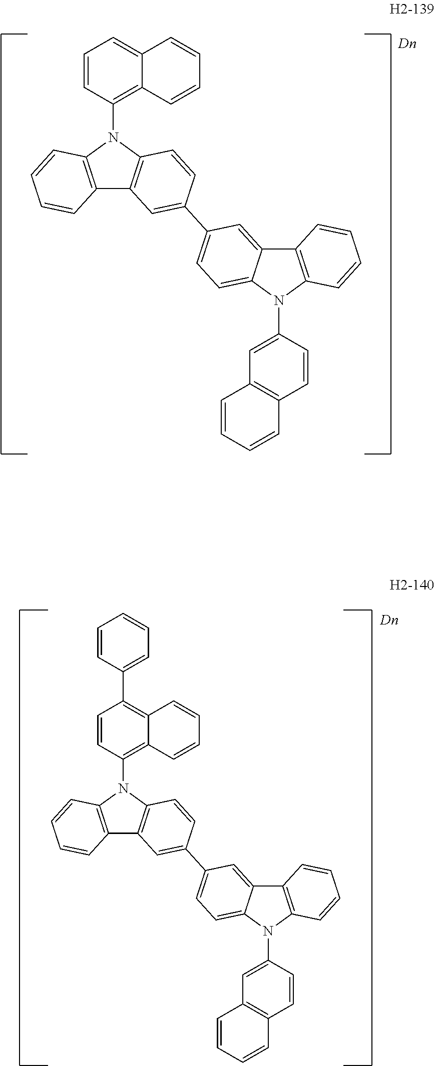 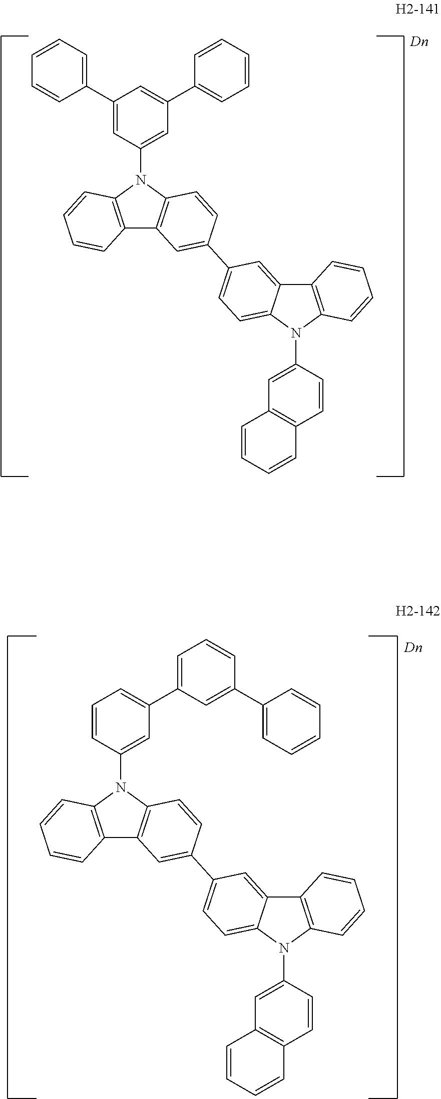 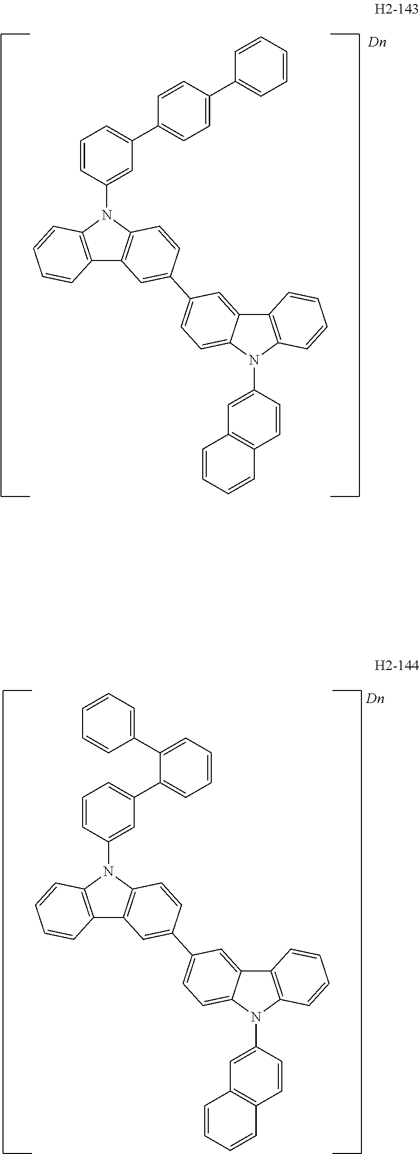 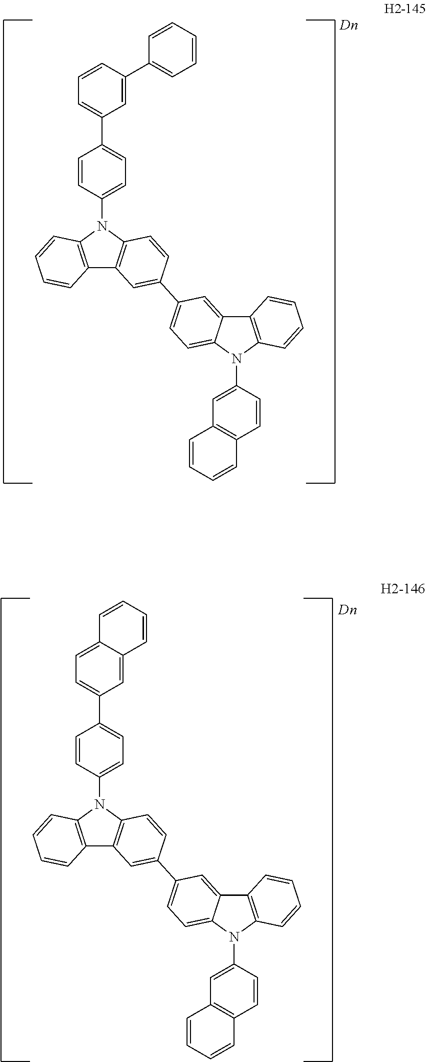 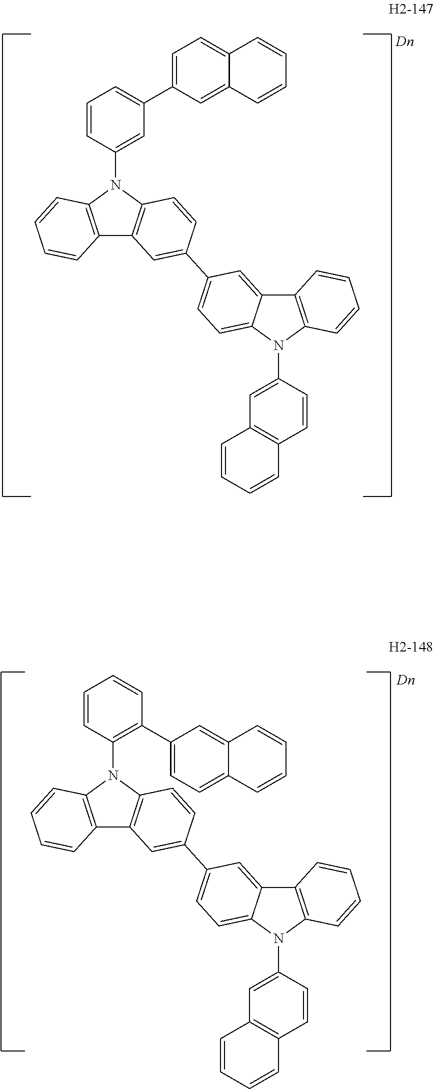 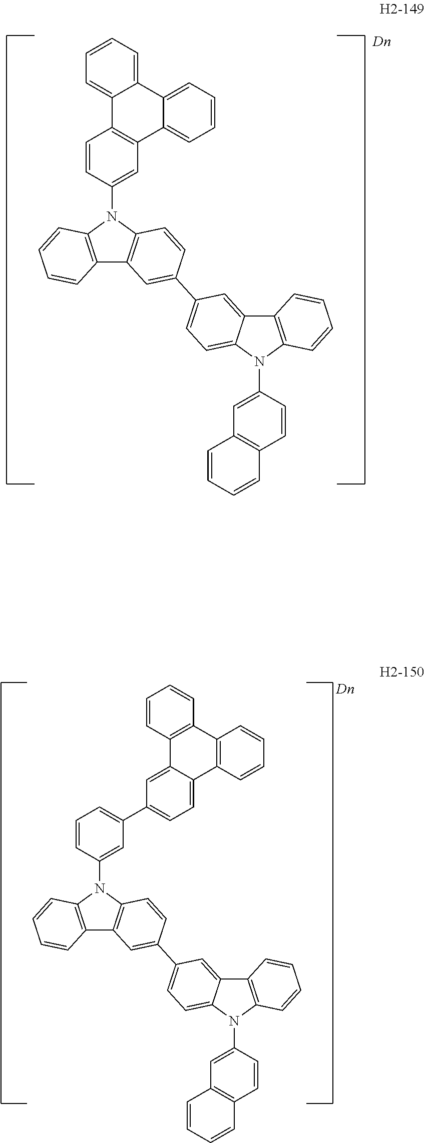 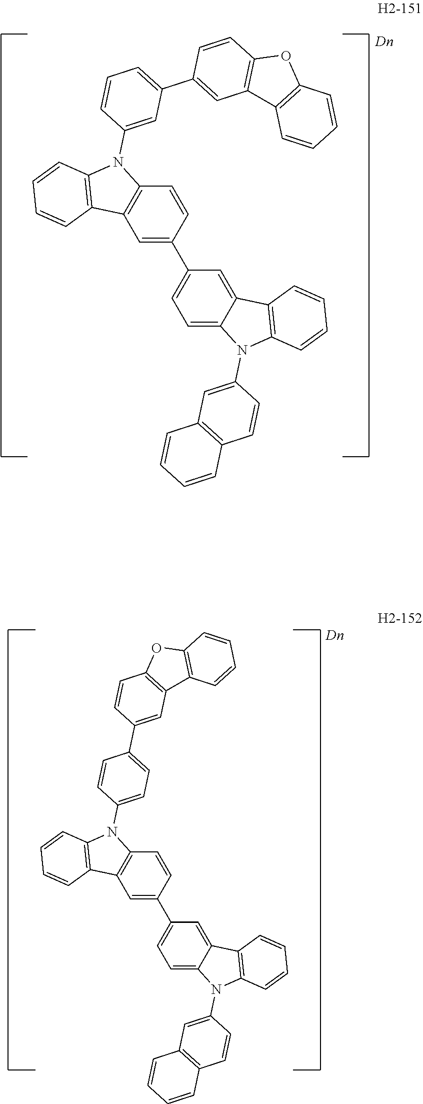 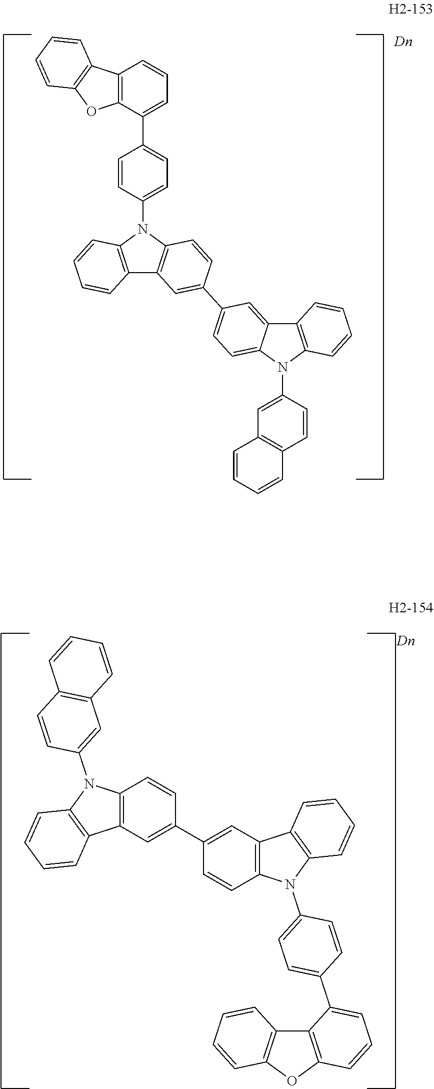 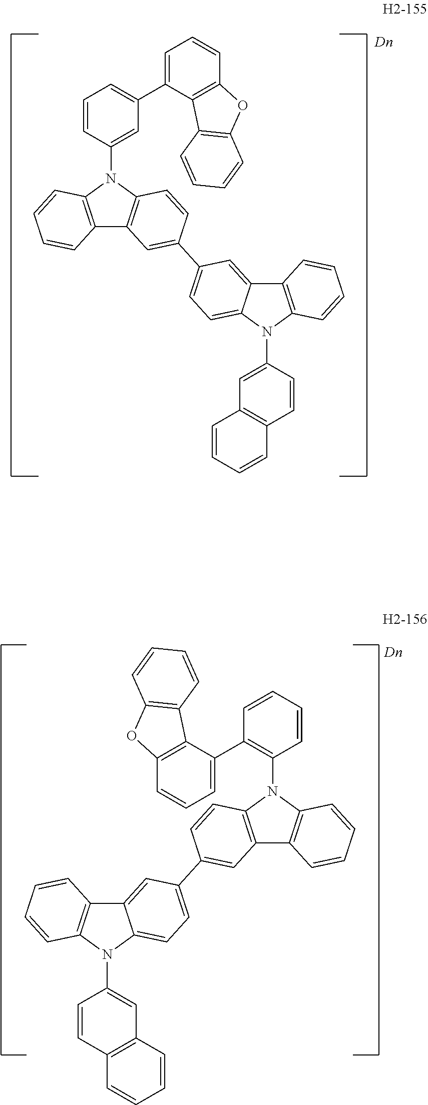 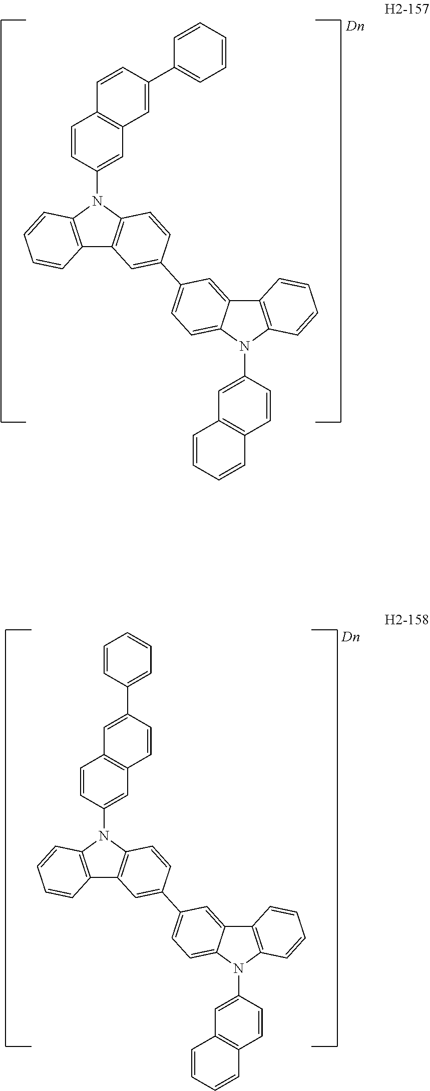 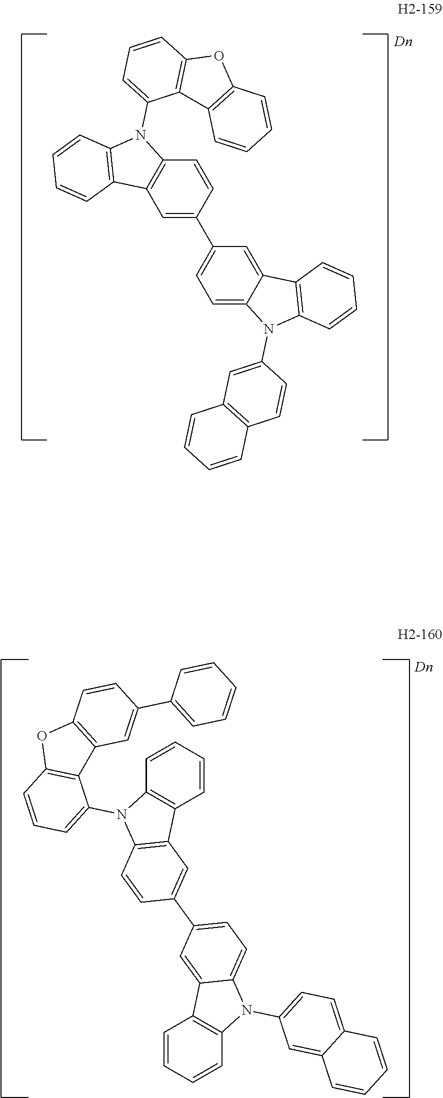 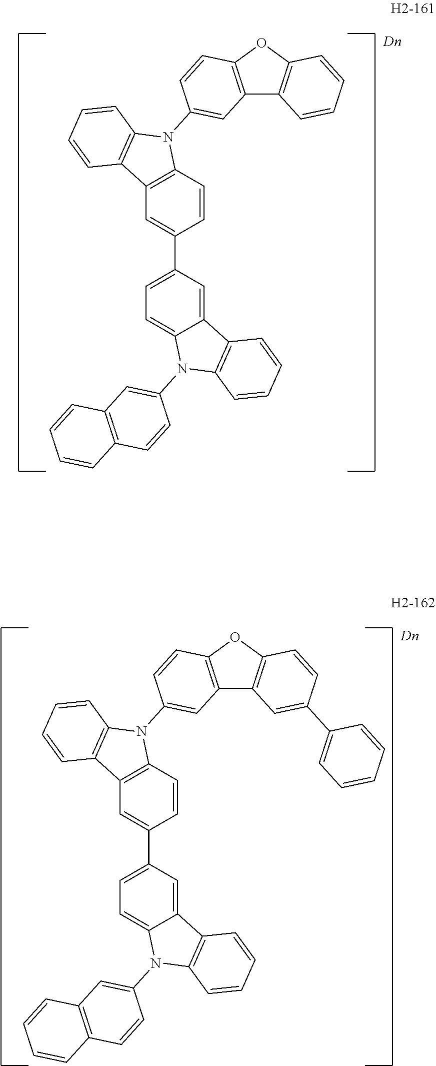 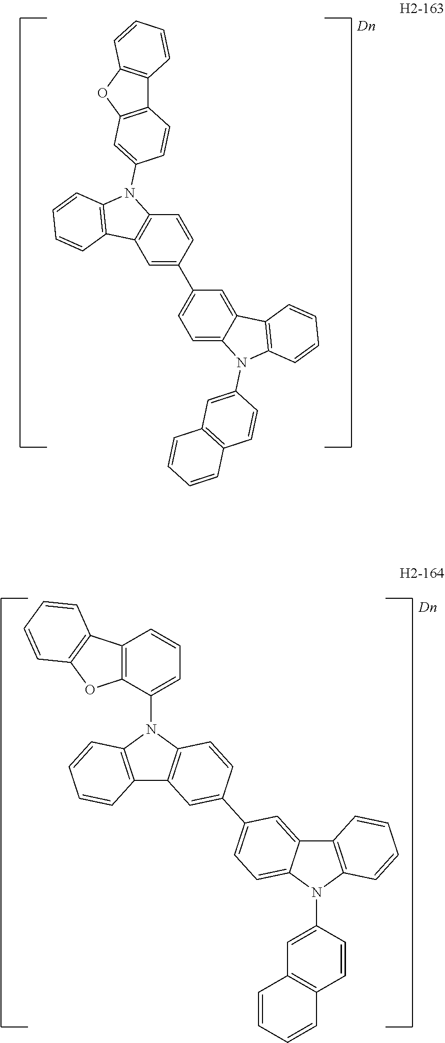 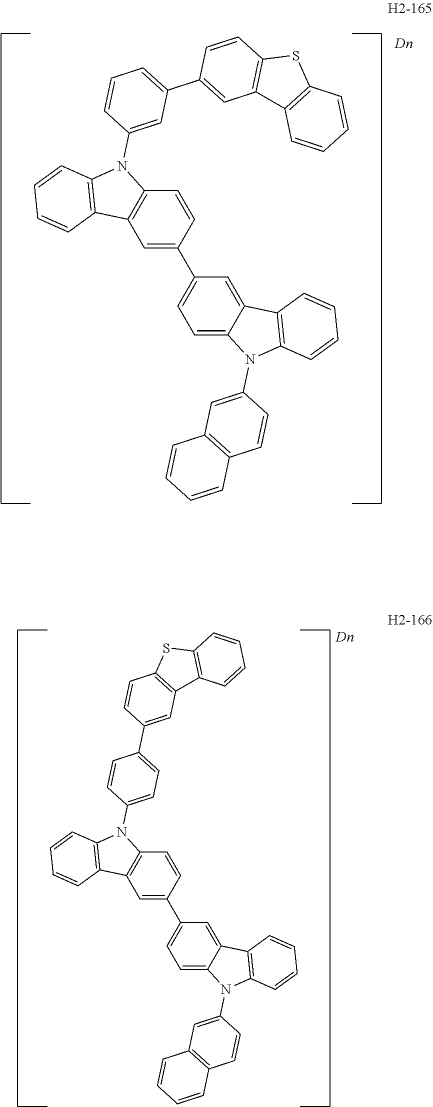 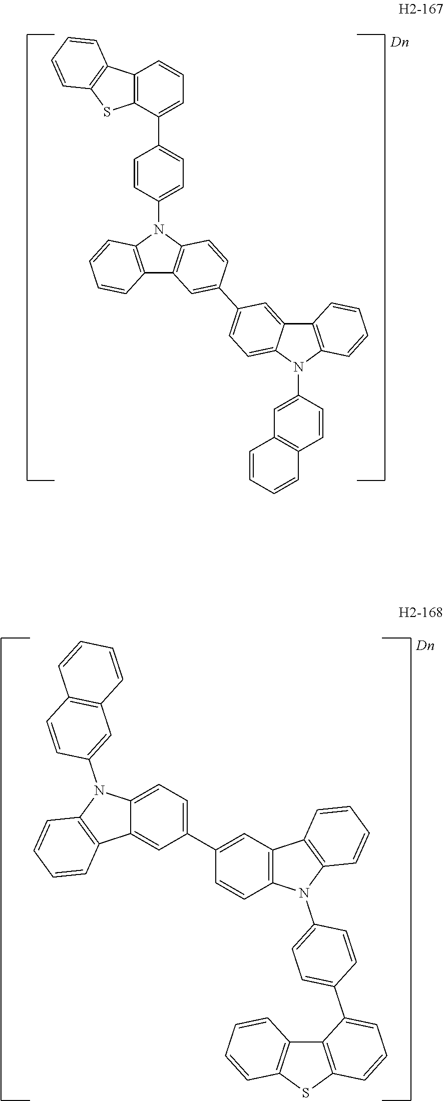 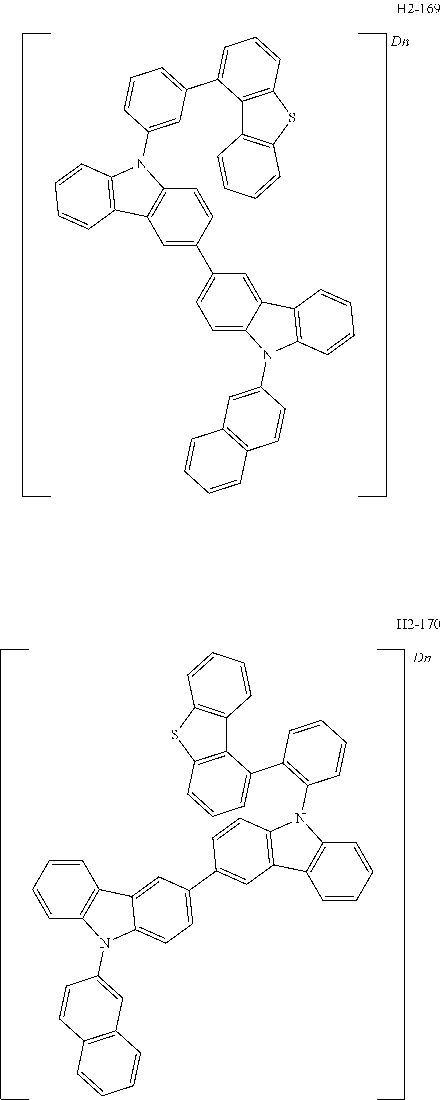 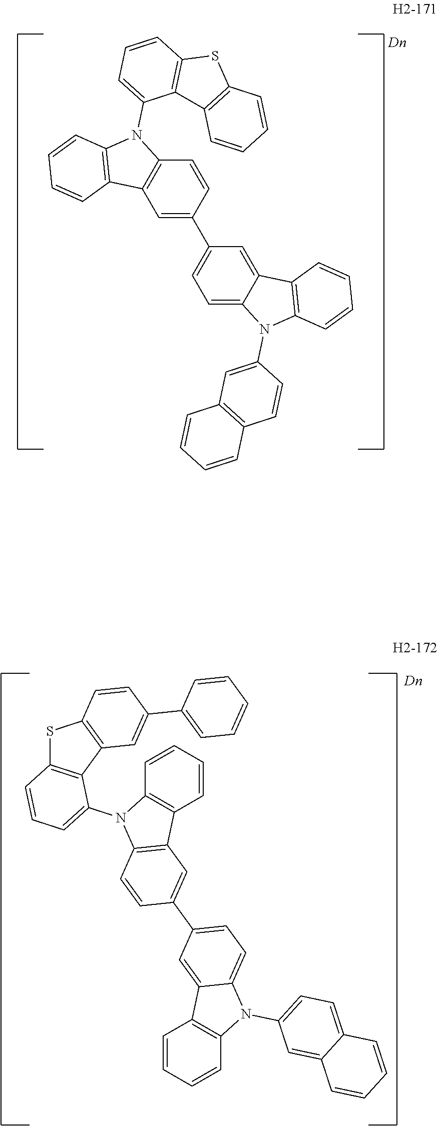 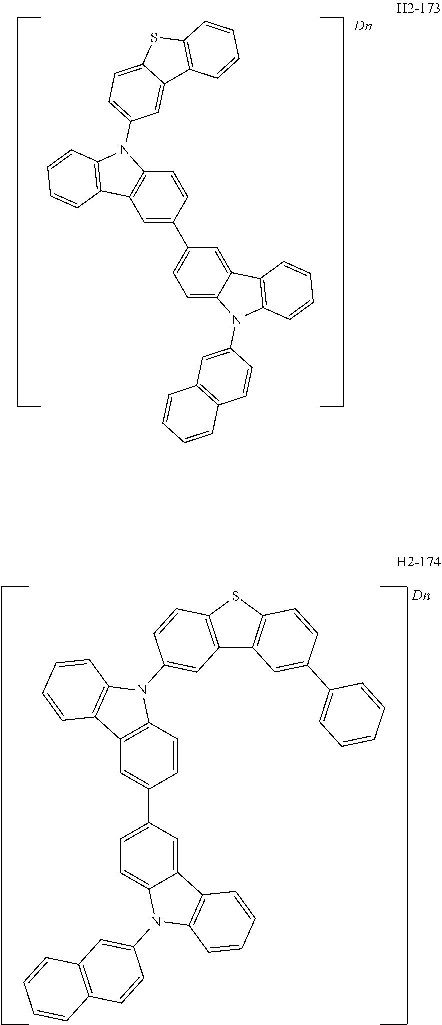 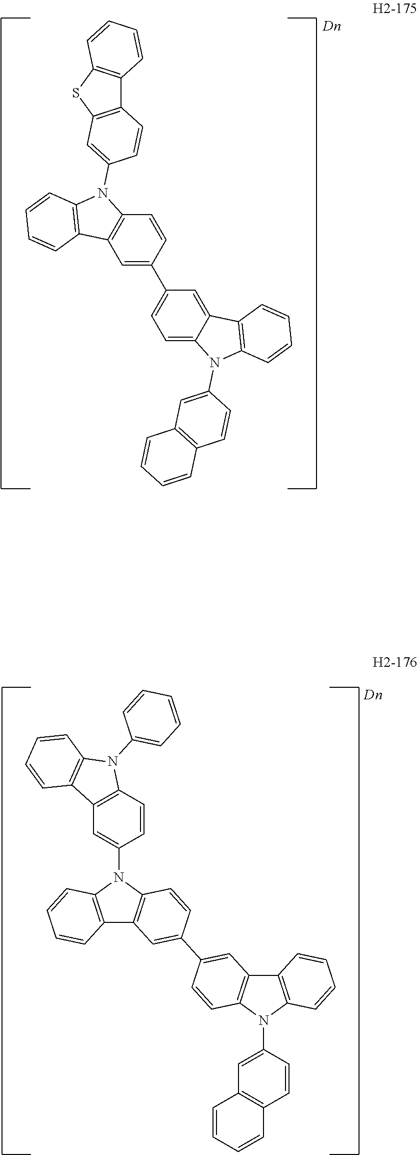 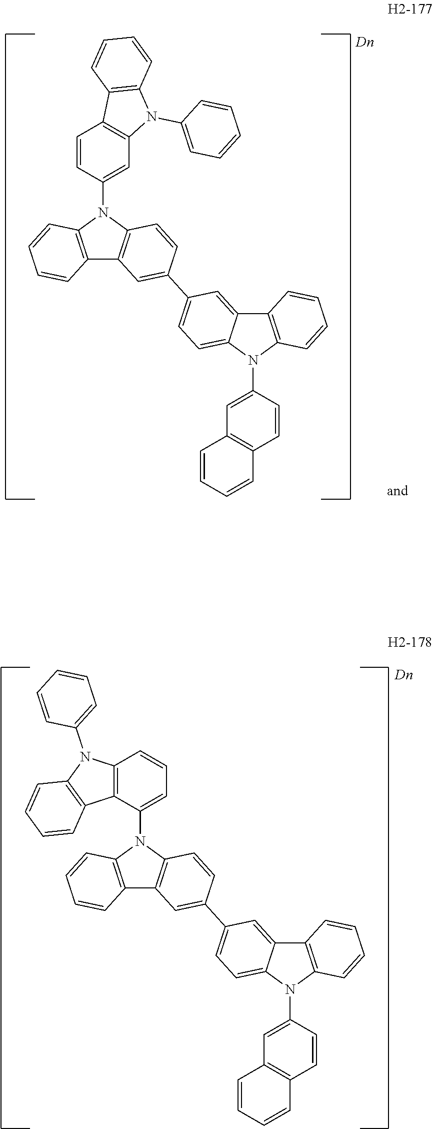
wherein, Dₙ represents that n number of hydrogens are replaced with deuterium; and n represents an integer of 4 to 50.

12. An organic electroluminescent device comprising an anode, a cathode, and at least one light-emitting layer between the anode and the cathode, wherein at least one of the light-emitting layers comprises the plurality of host materials according to claim 1.

13. An organic electroluminescent compound represented by the following formula 2′: 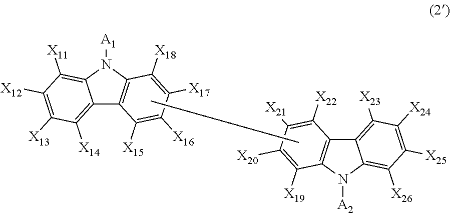
in formula 2′,
A₁ and A₂, each independently, represent a substituted or unsubstituted (C6-C30)aryl, a substituted or unsubstituted dibenzofuranyl, a substituted or unsubstituted dibenzothiophenyl, or a substituted or unsubstituted carbazolyl; and
X₁₁ to X₂₆, each independently, represent hydrogen, deuterium, a substituted or unsubstituted (C6-C30)aryl, or a substituted or unsubstituted (3- to 30-membered)heteroaryl;
any one of X₁₁ to X₁₈ is linked to any one of X₁₉ to X₂₆ to form a single bond;
with the proviso that at least four of X₁₁ to X₂₆ are deuterium, and at least one of X₁₁, X₁₈, X₁₉, and X₂₆ is deuterium.

14. The organic electroluminescent compound according to claim 13, wherein the compound represented by formula 2′ is selected from the following compounds: 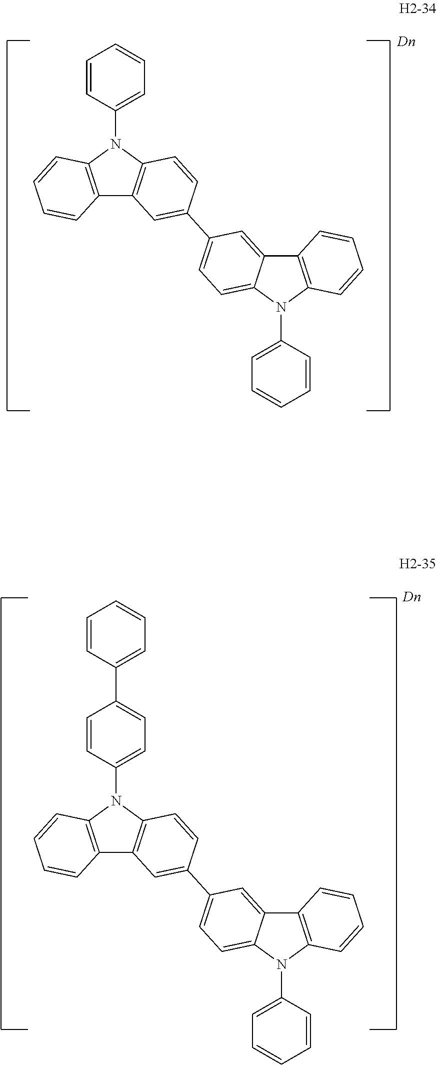 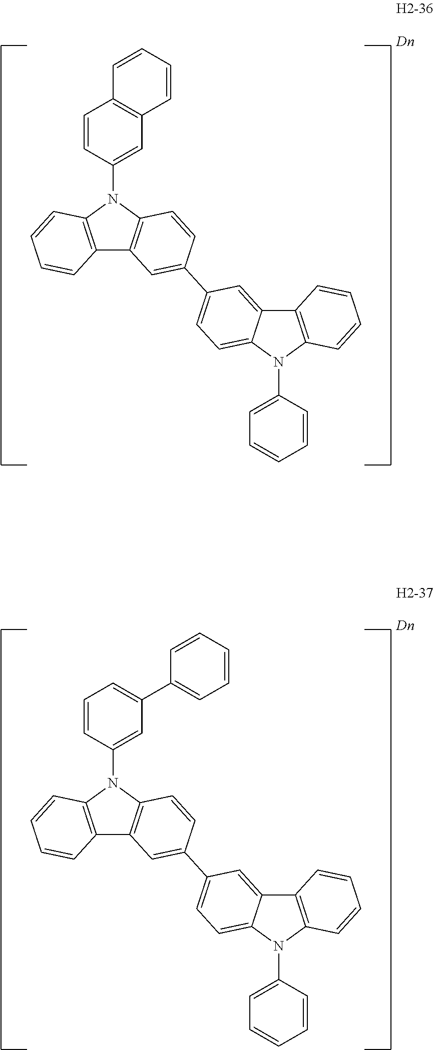 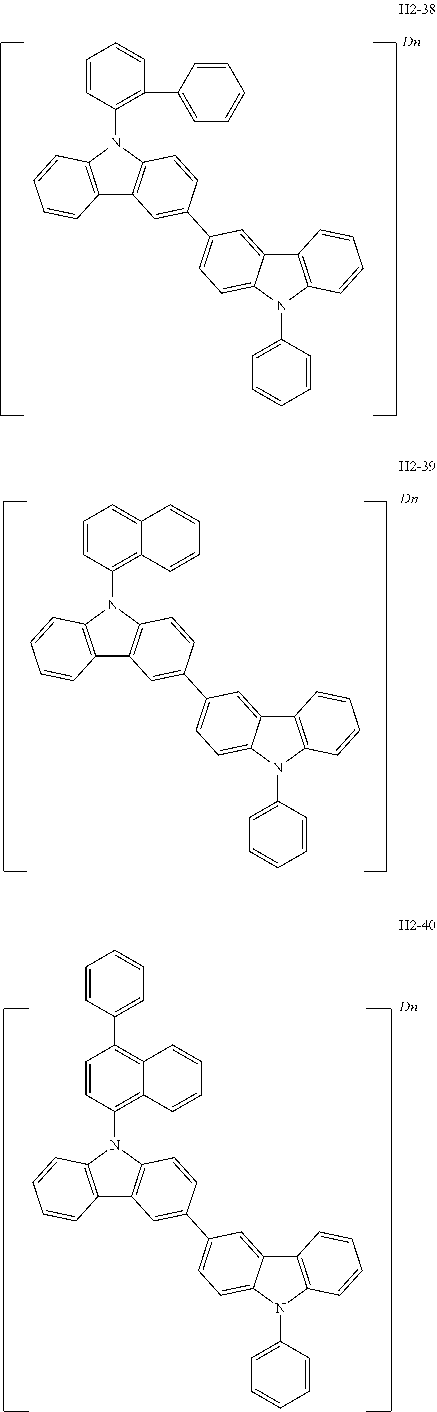 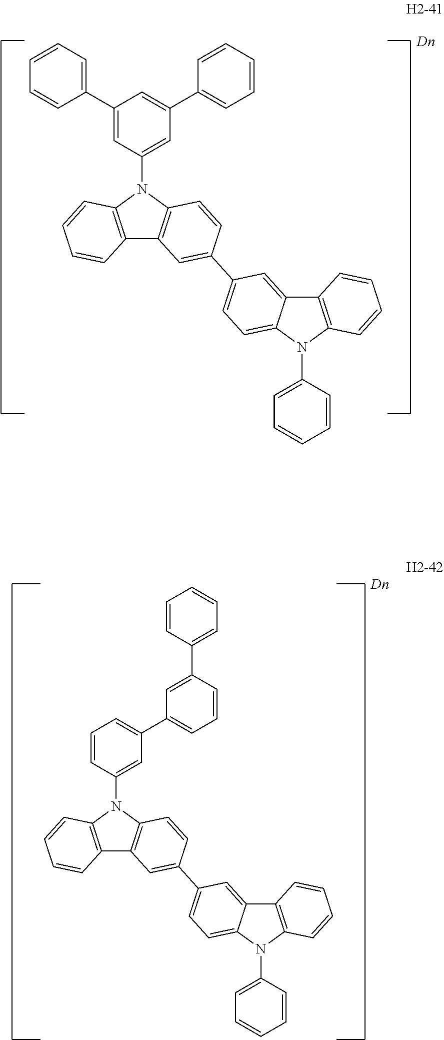 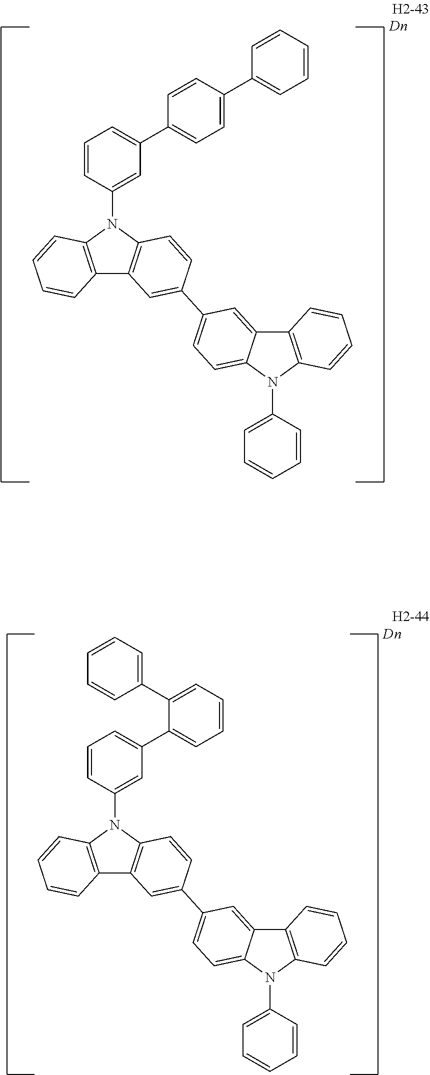 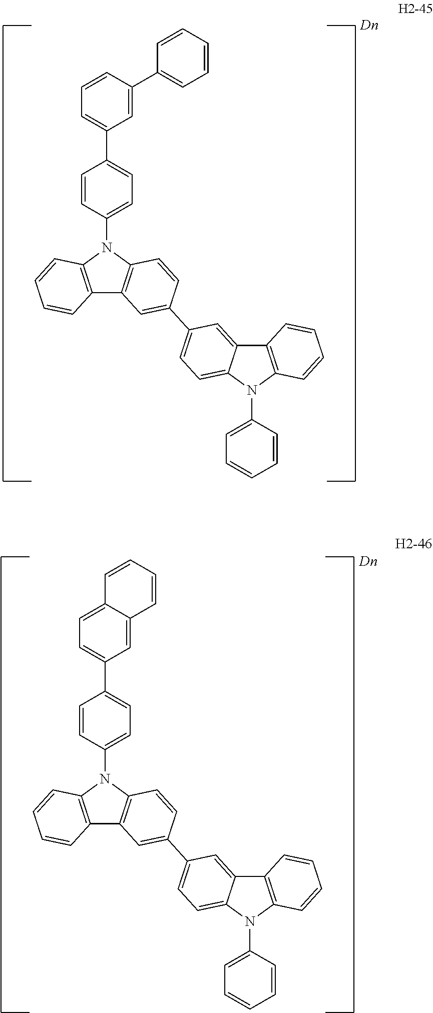 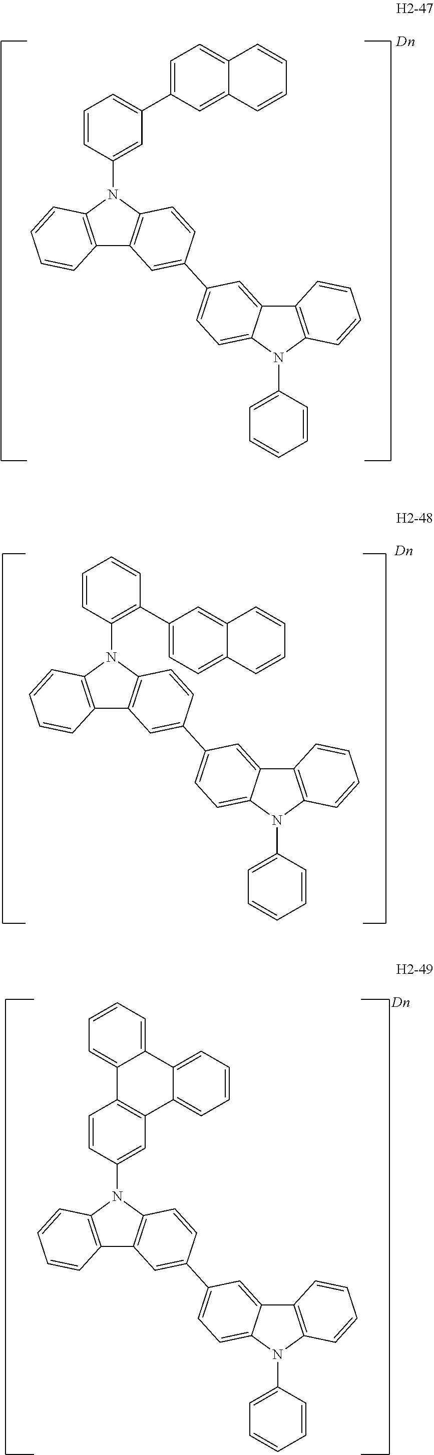 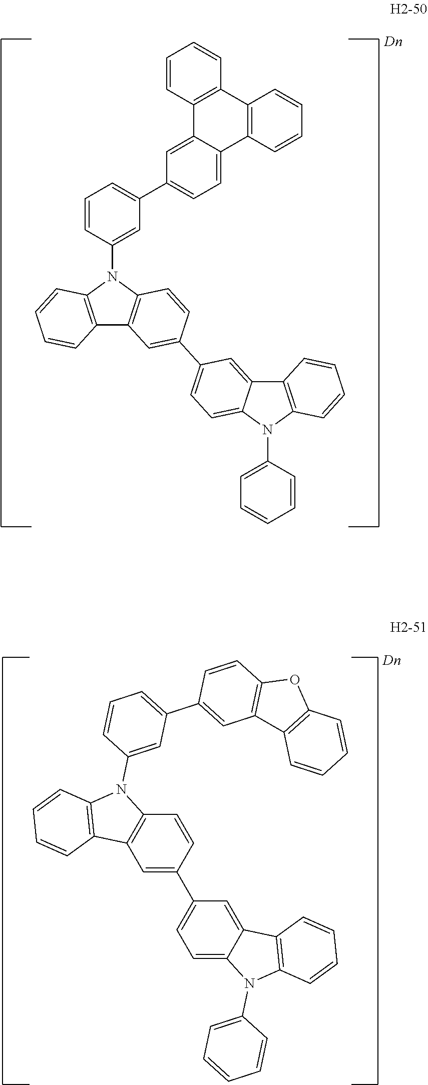 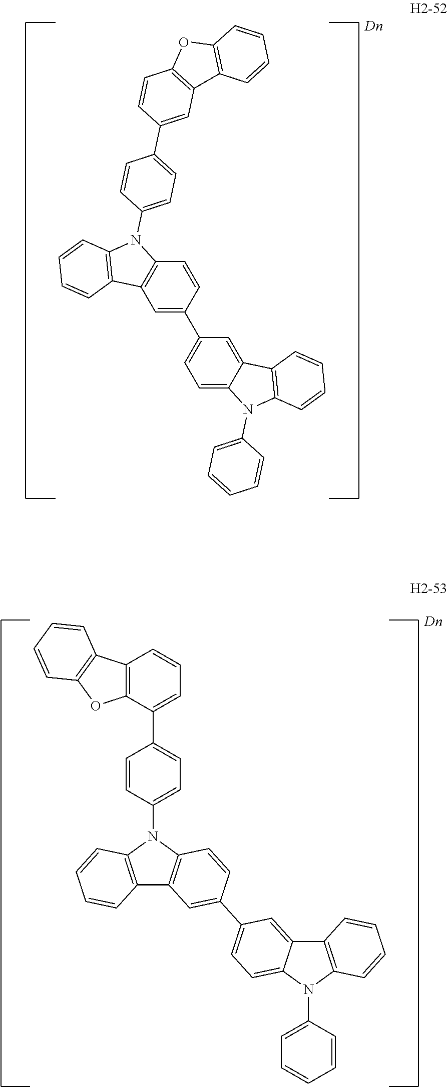 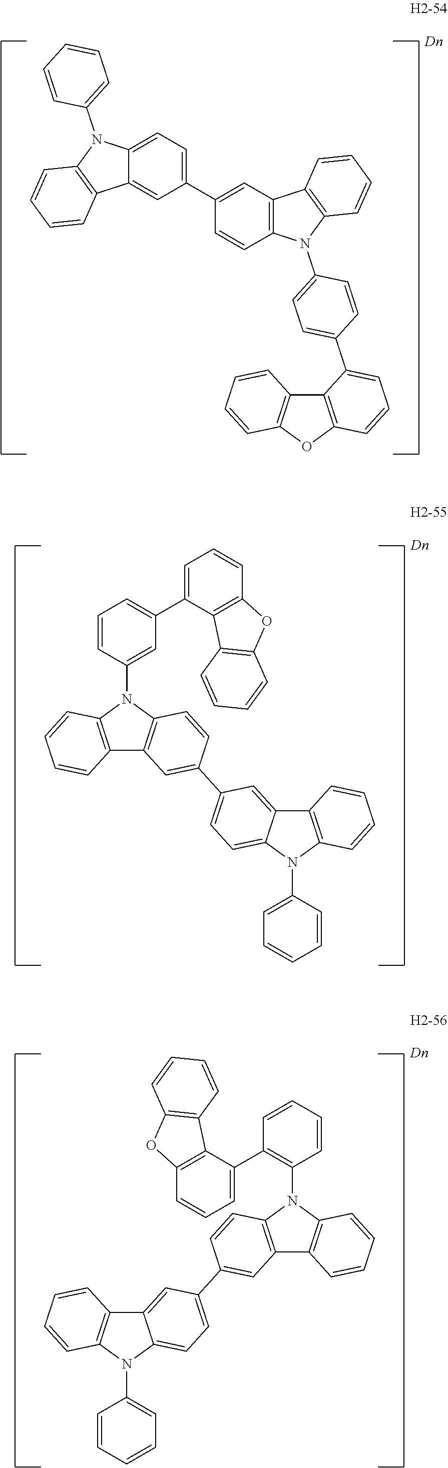 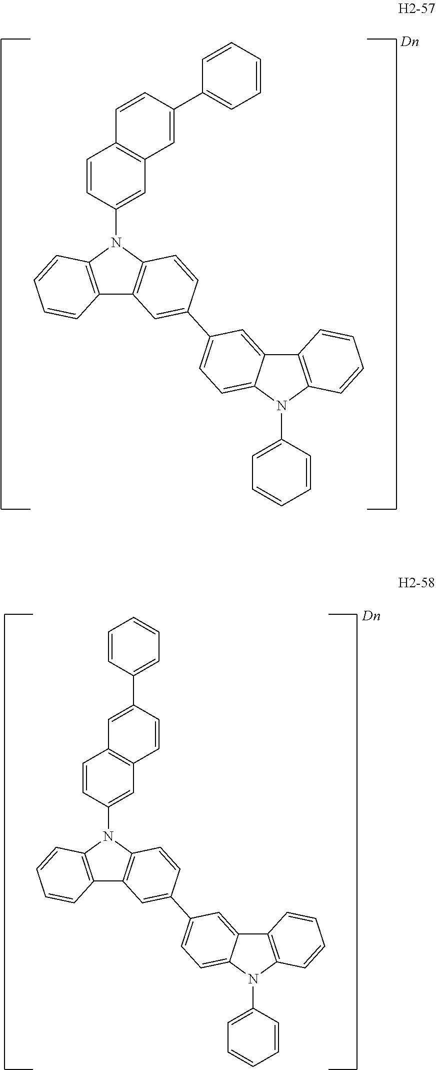 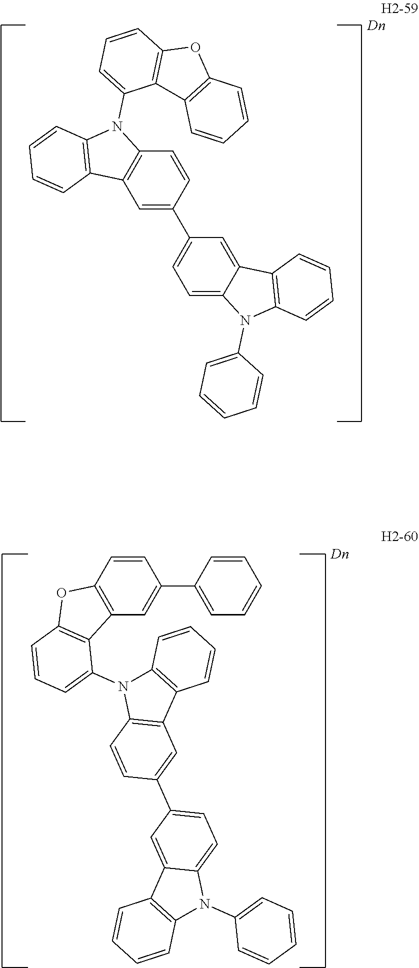 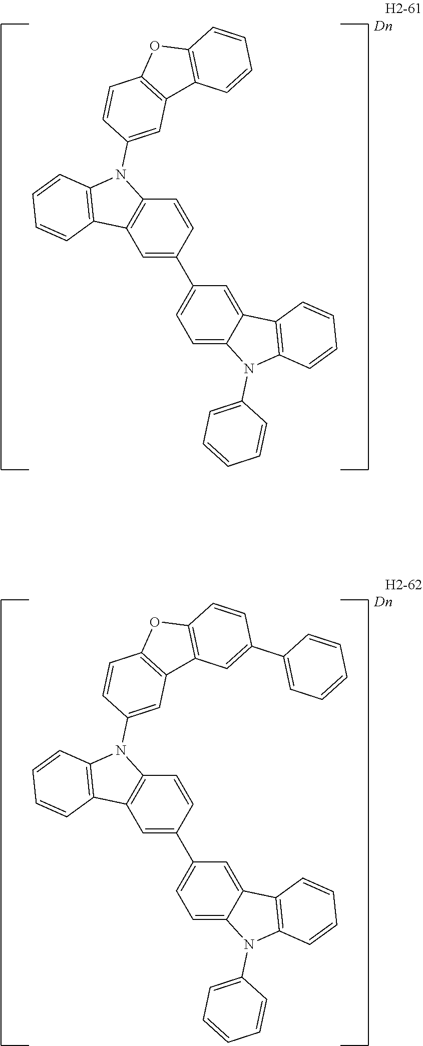 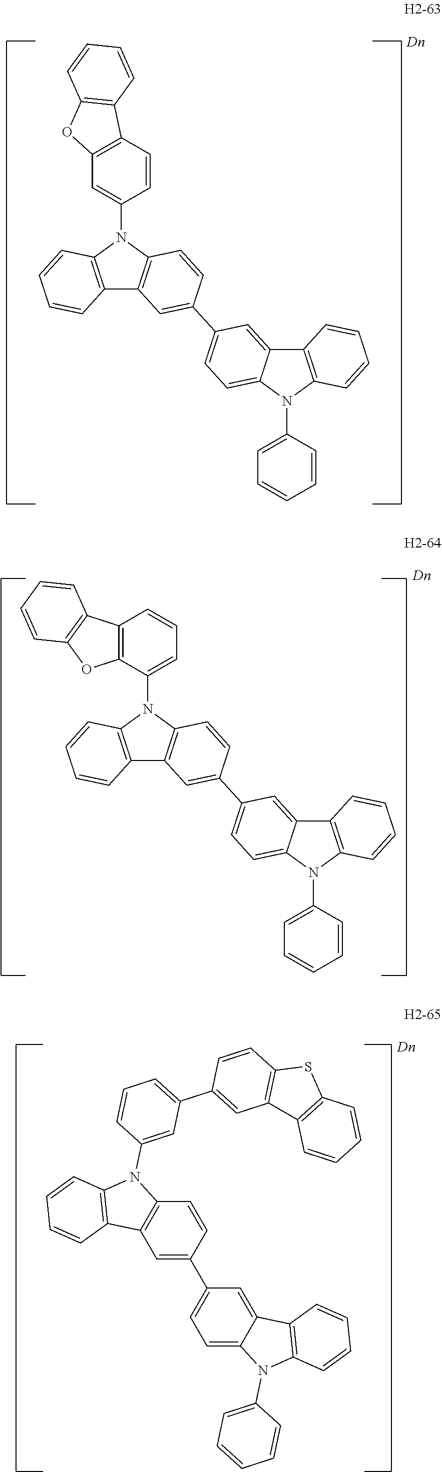 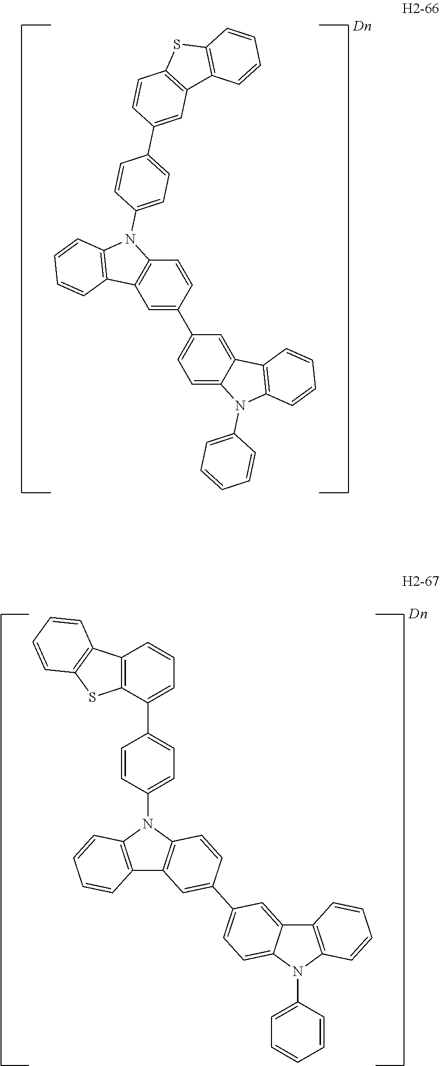 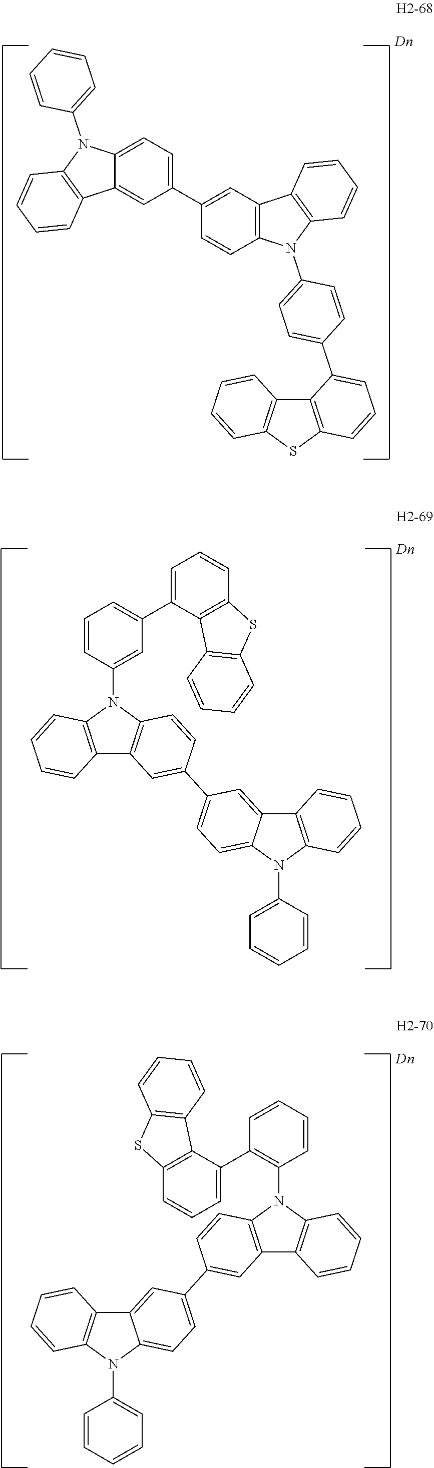 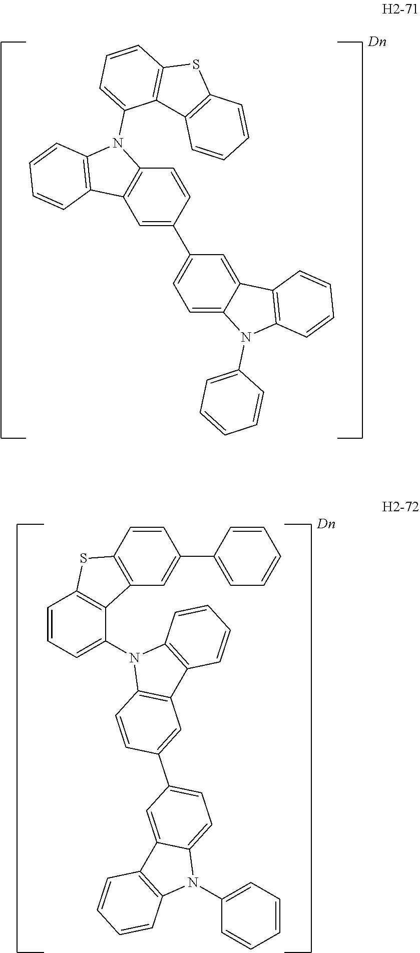 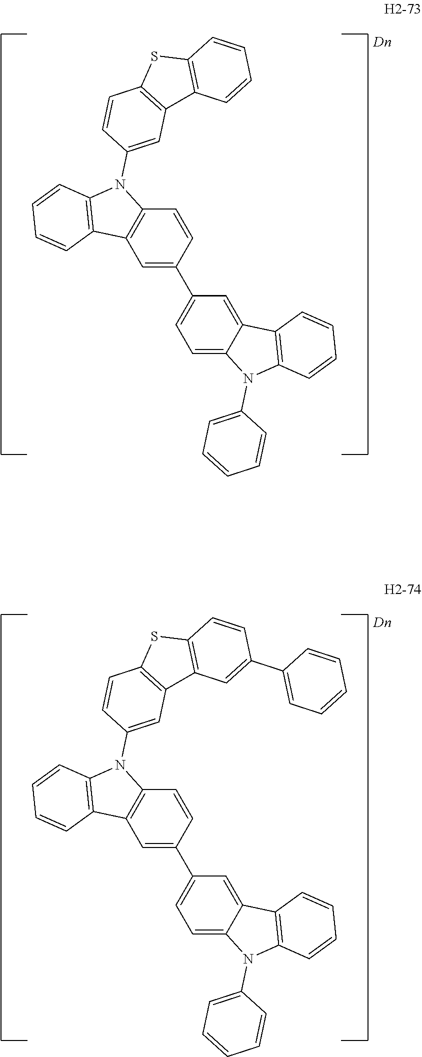 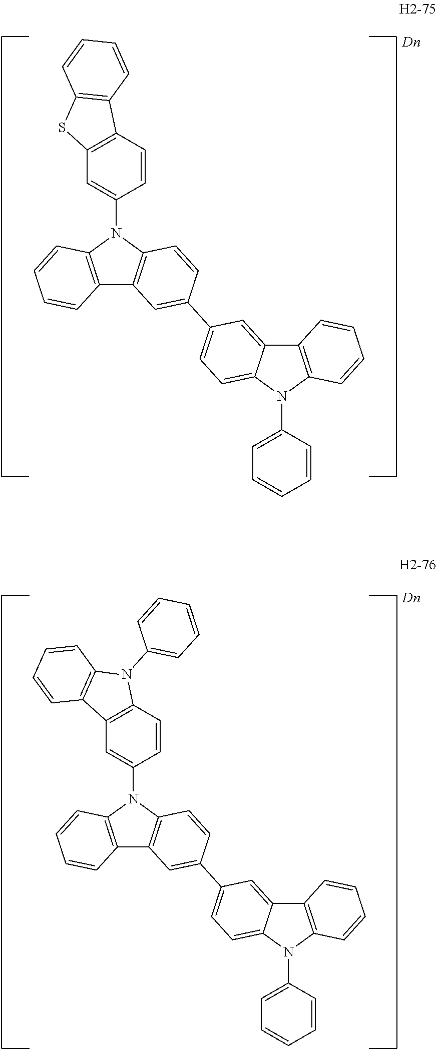 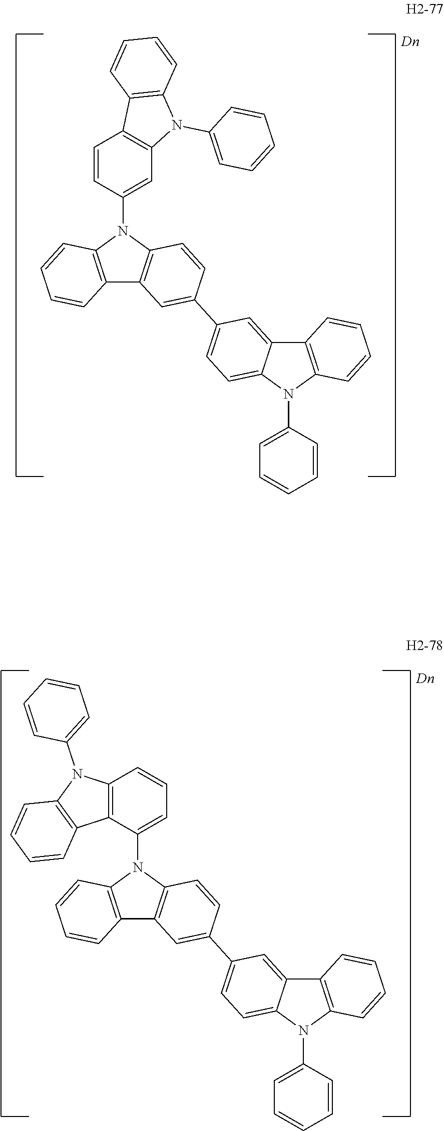 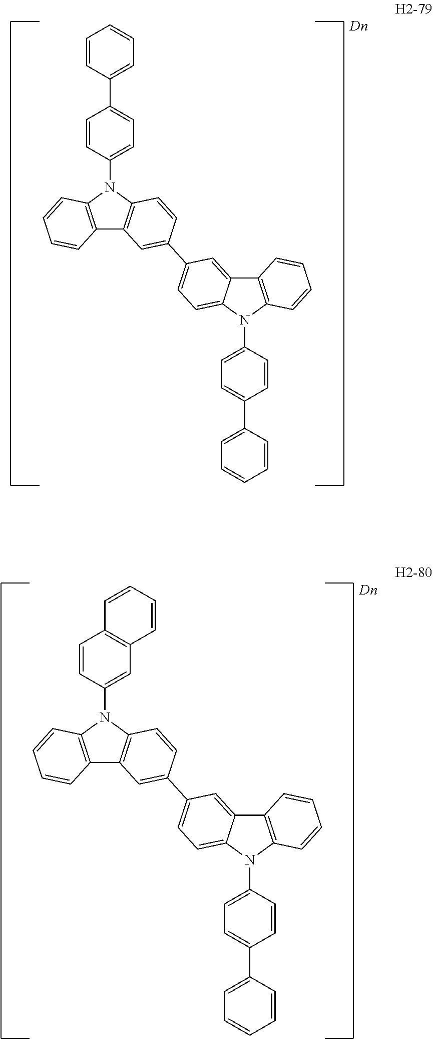 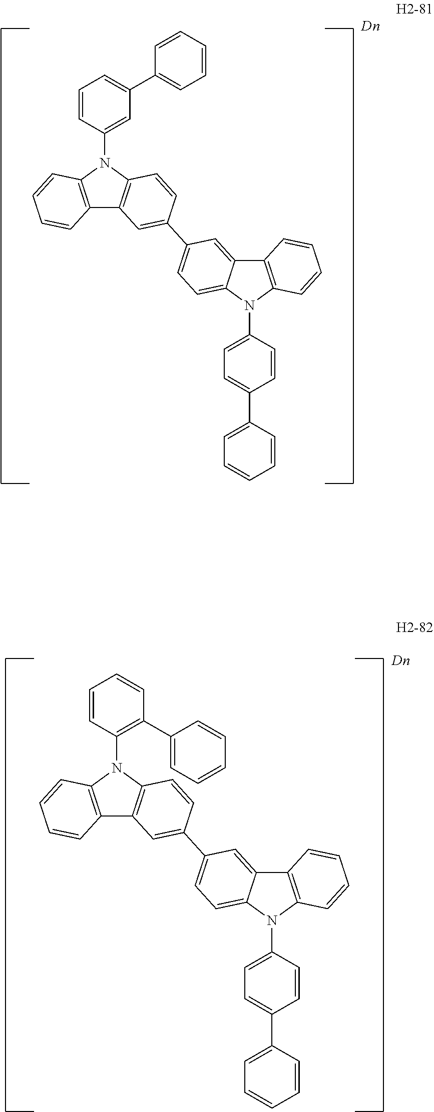 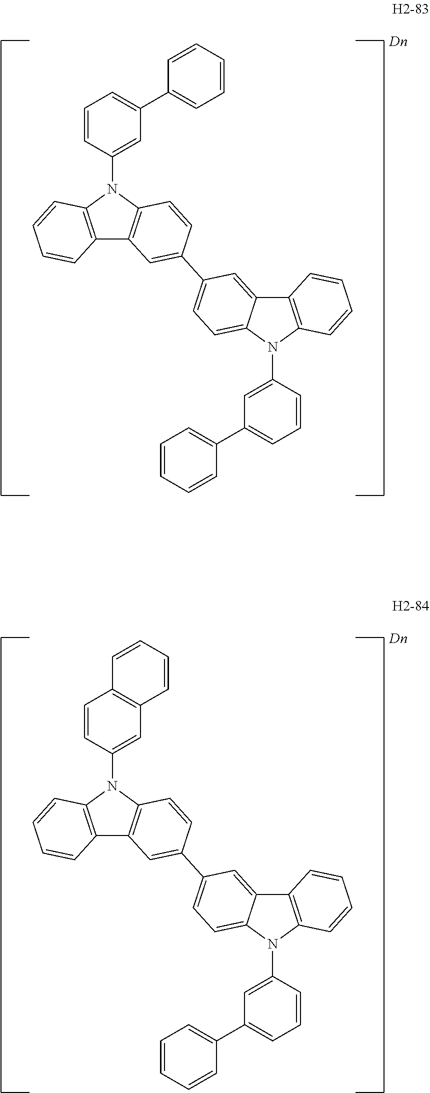 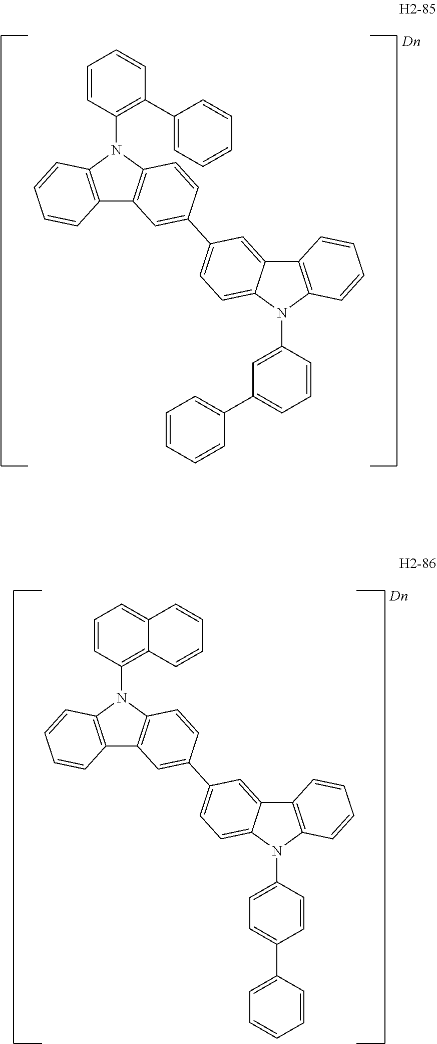 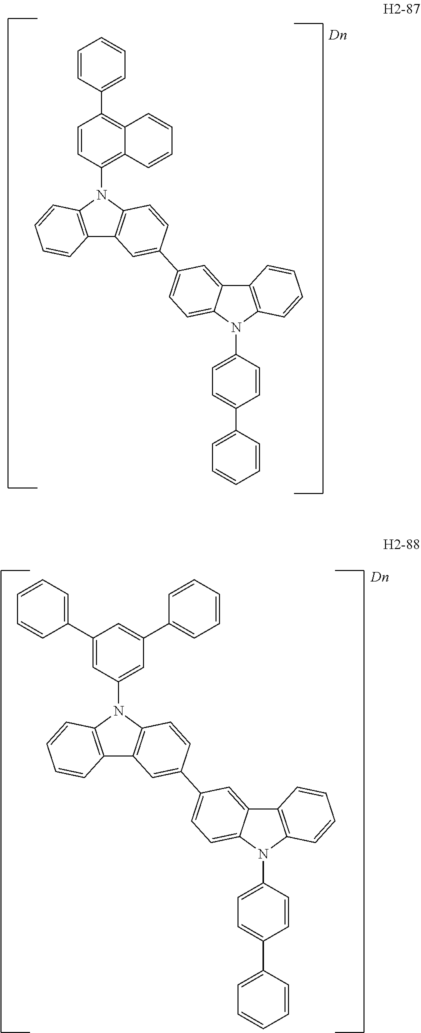 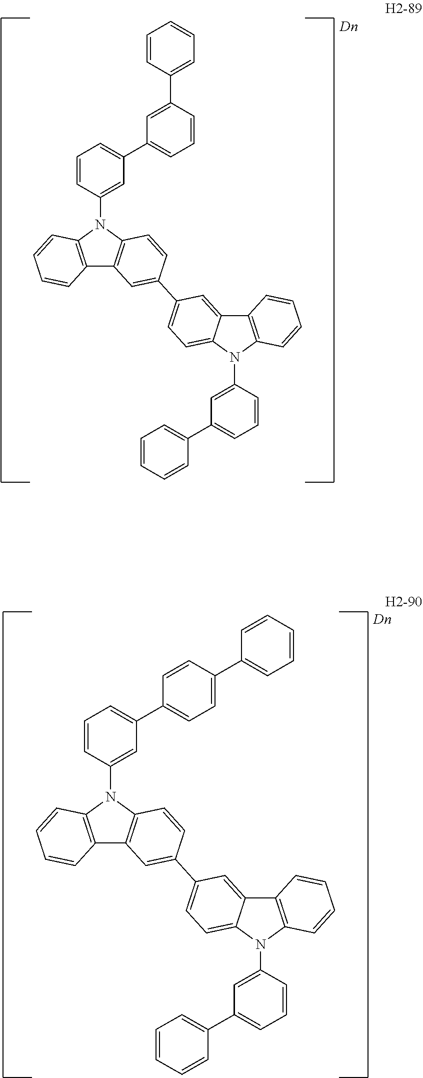 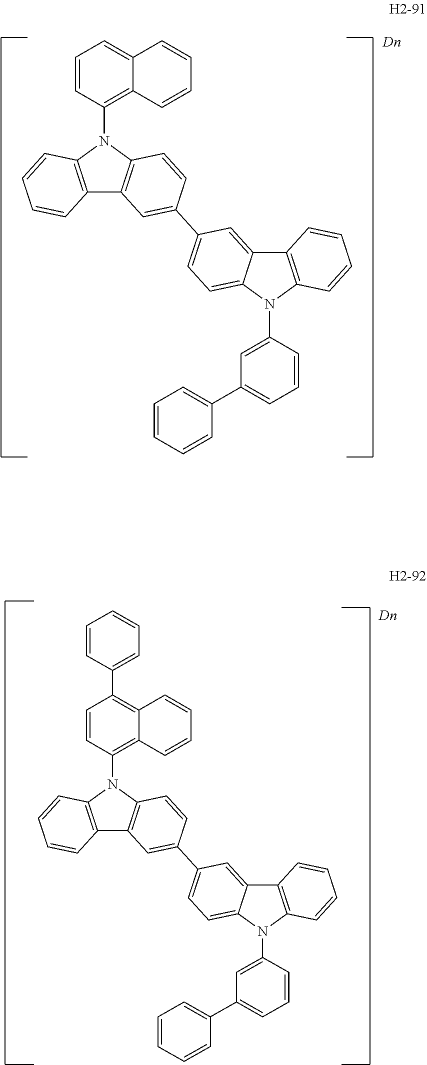 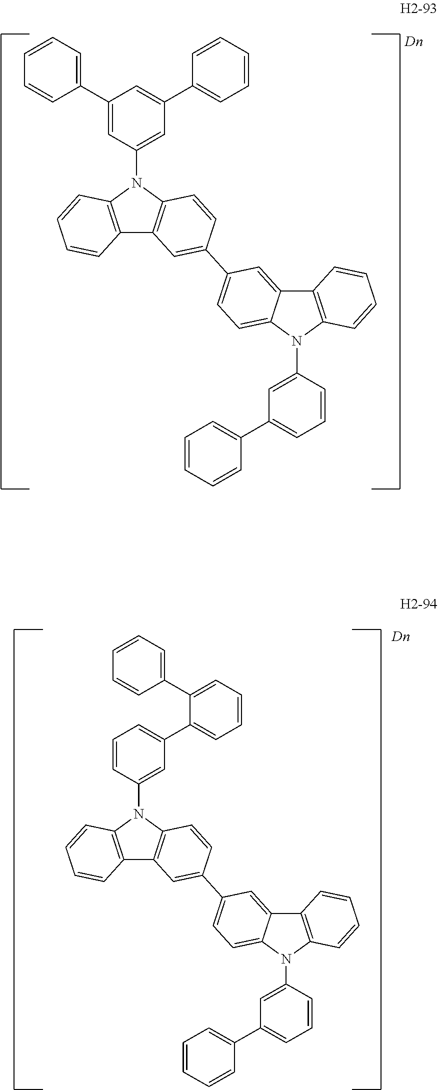 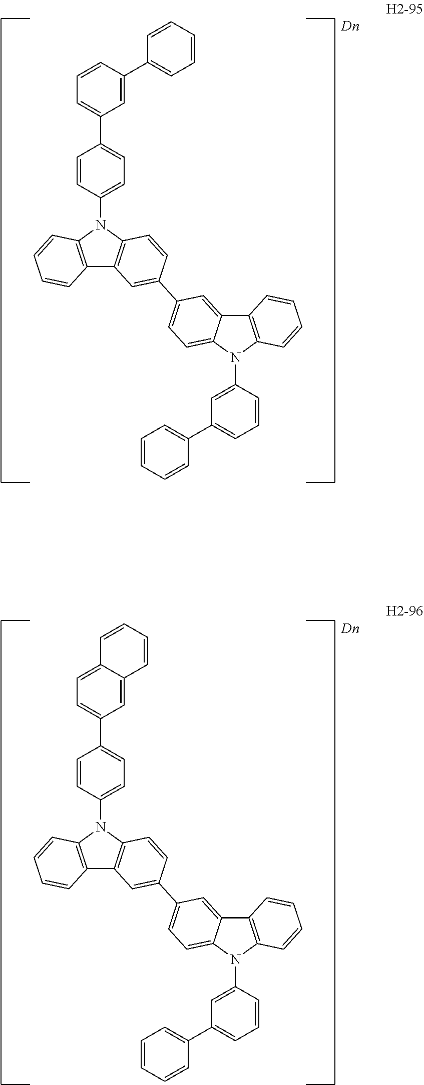 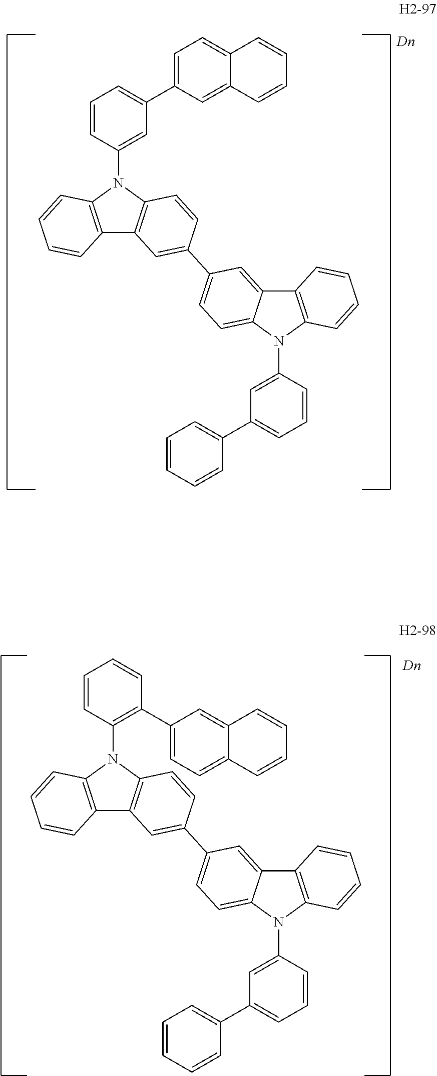 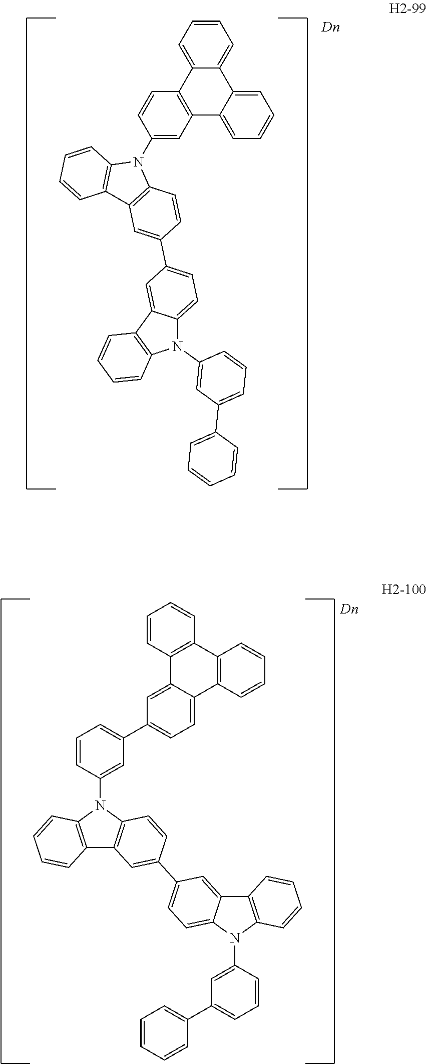 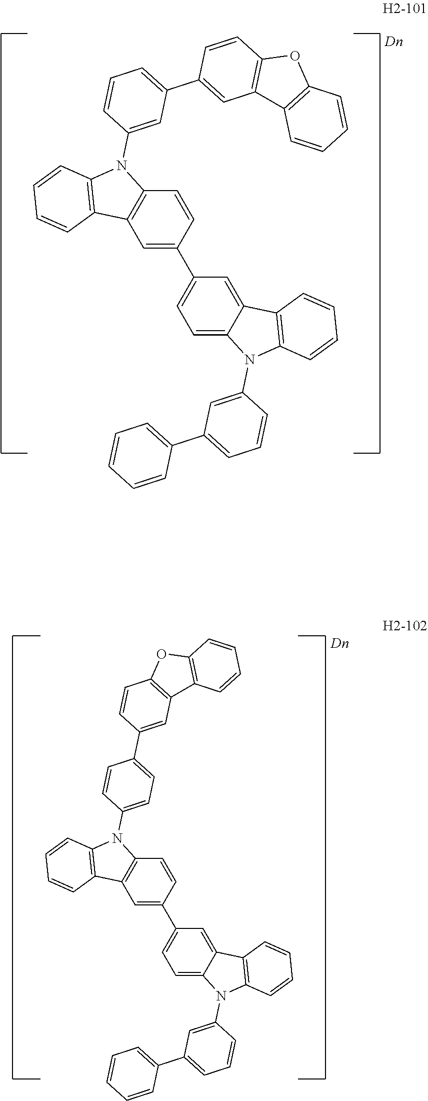 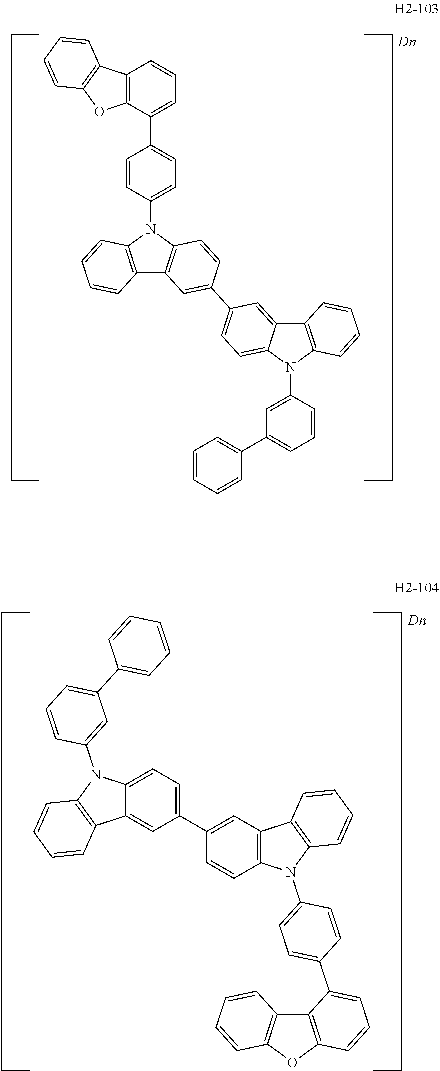 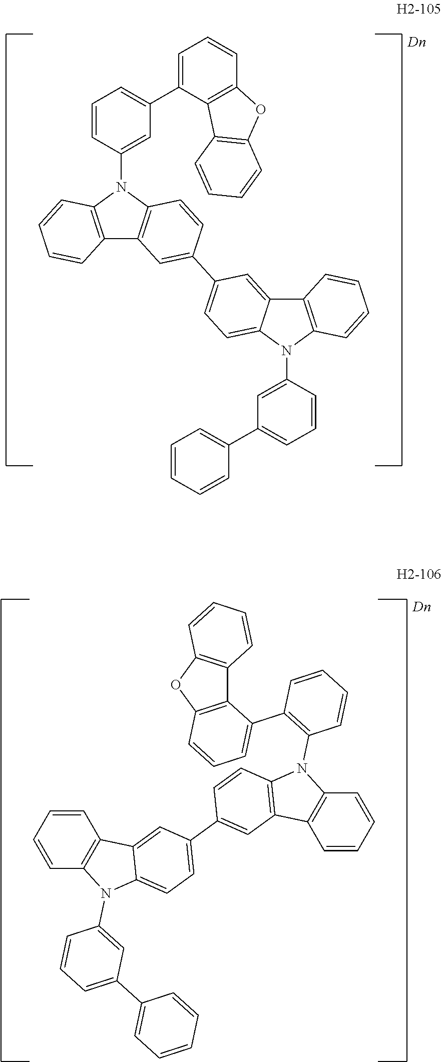 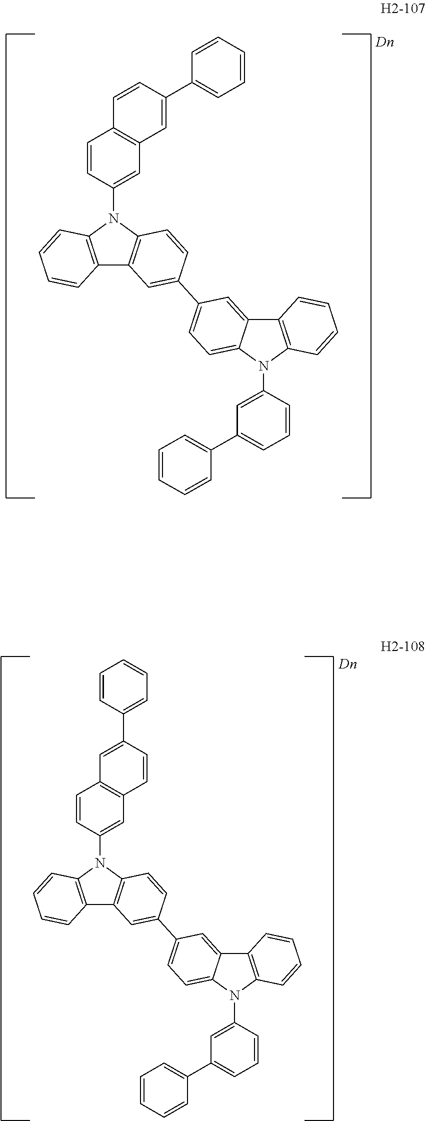 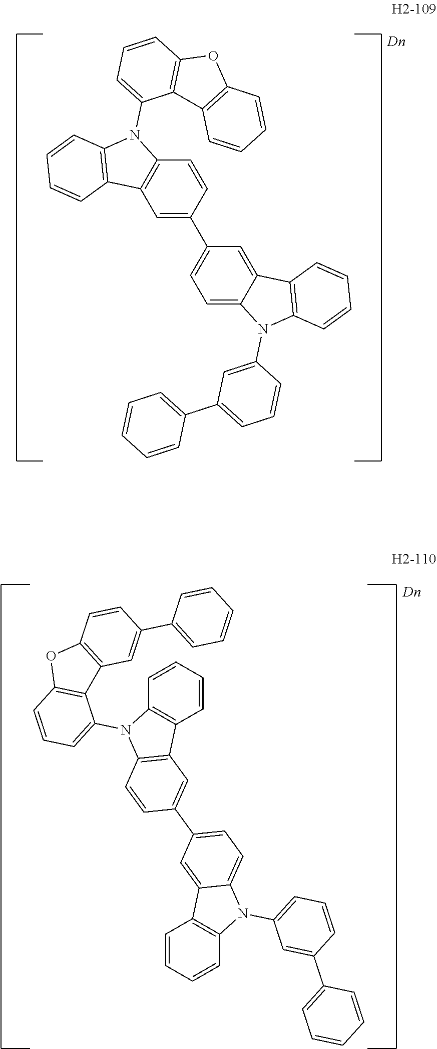 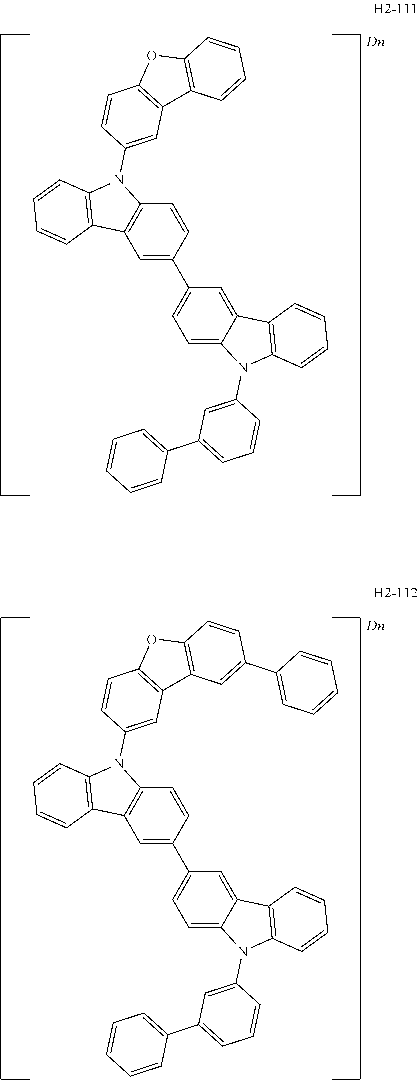 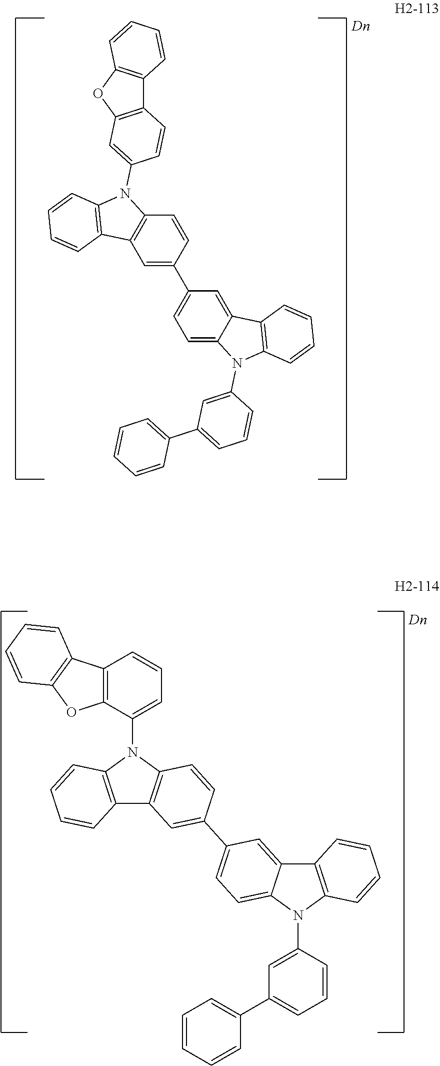 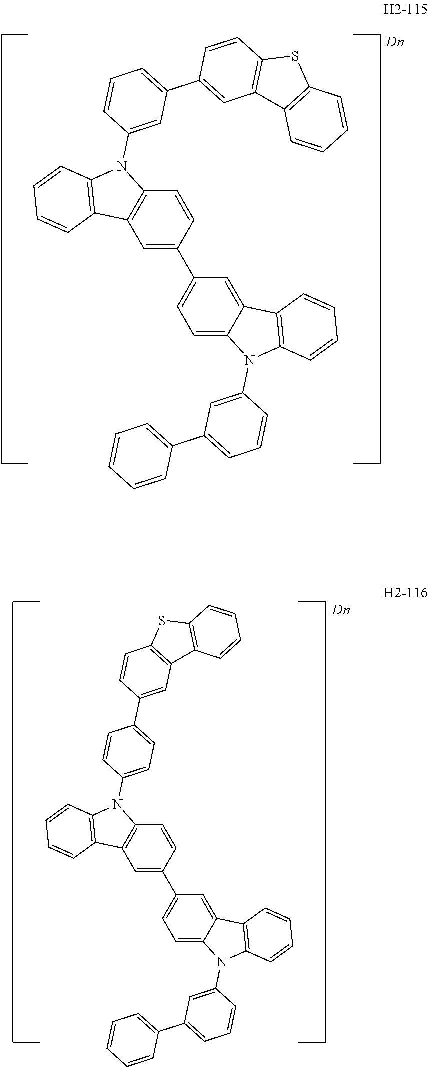 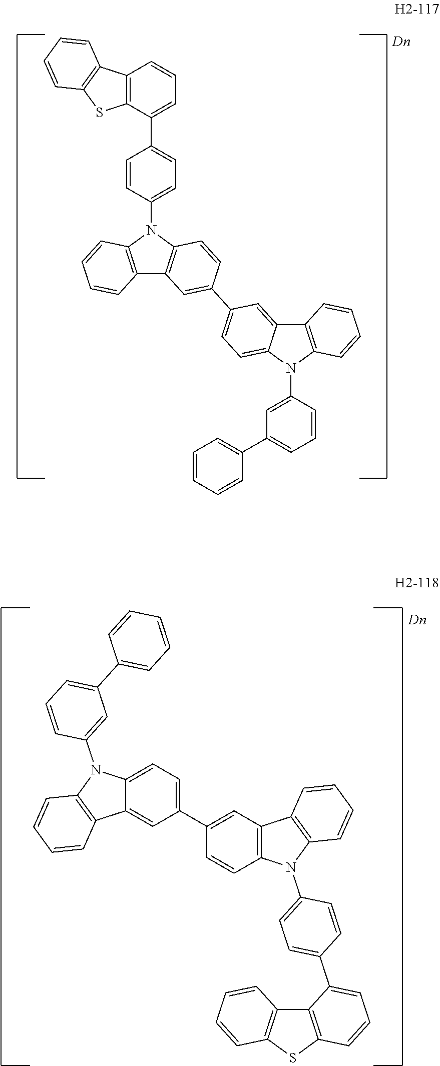 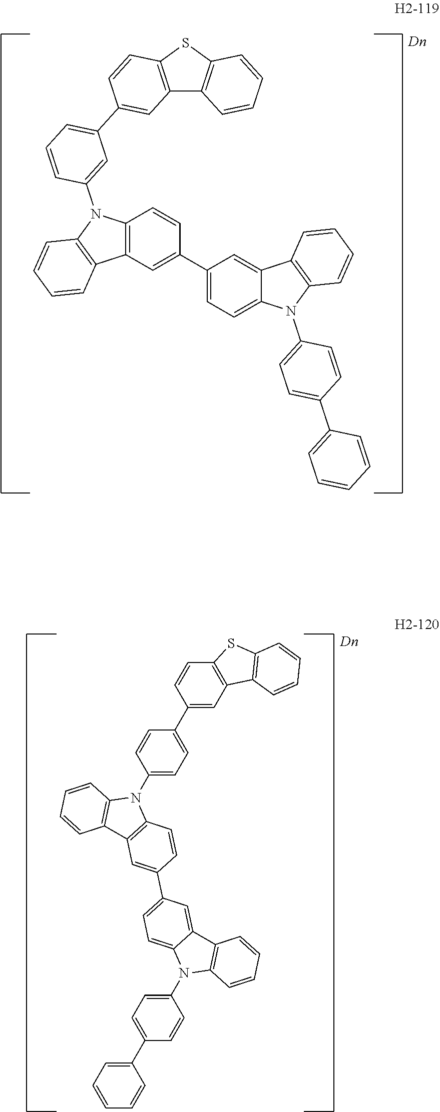 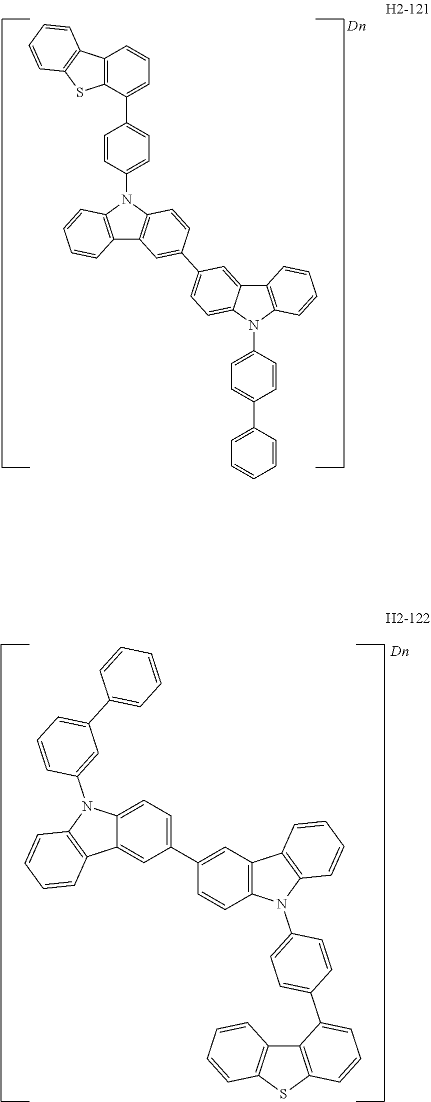 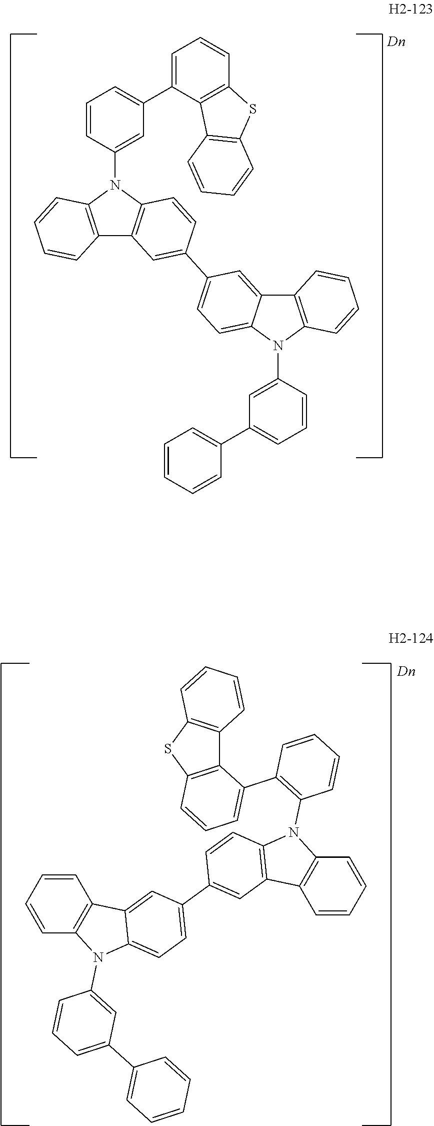 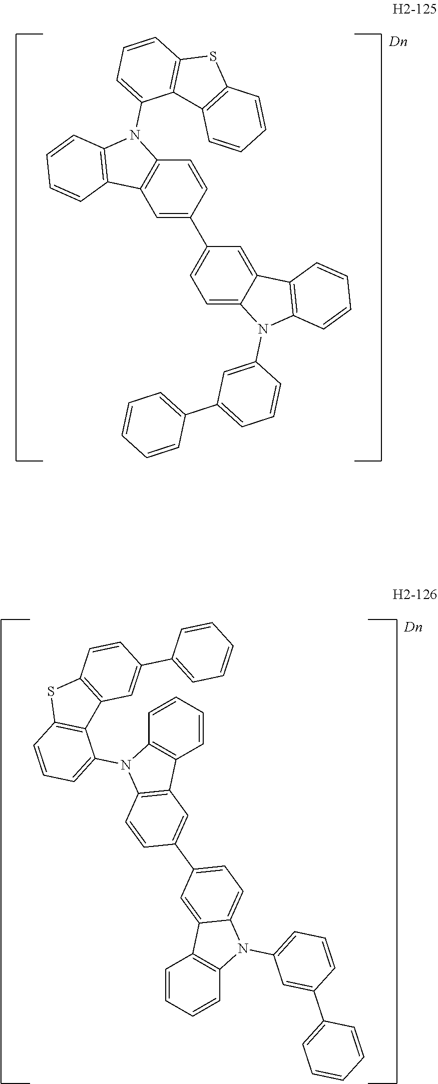 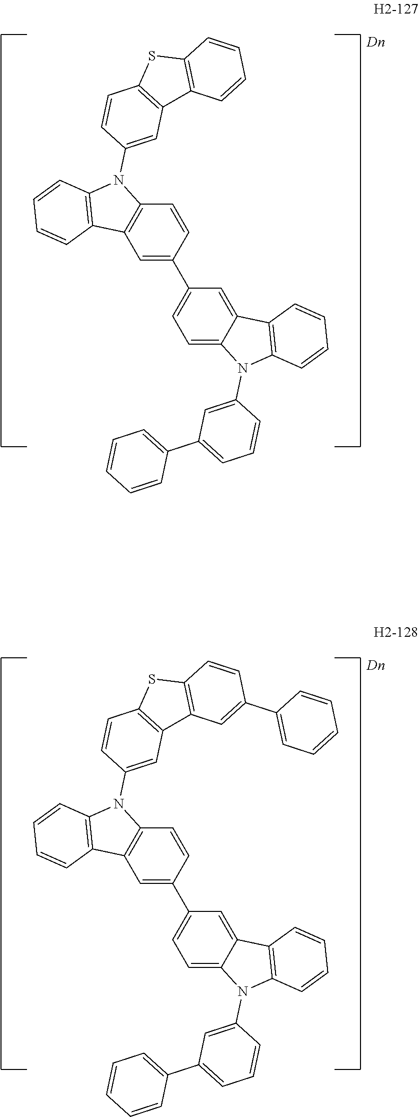 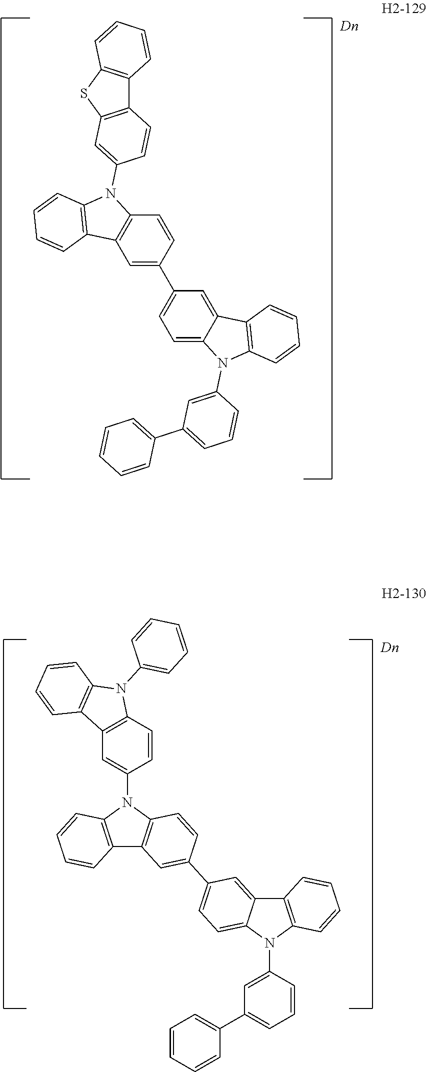 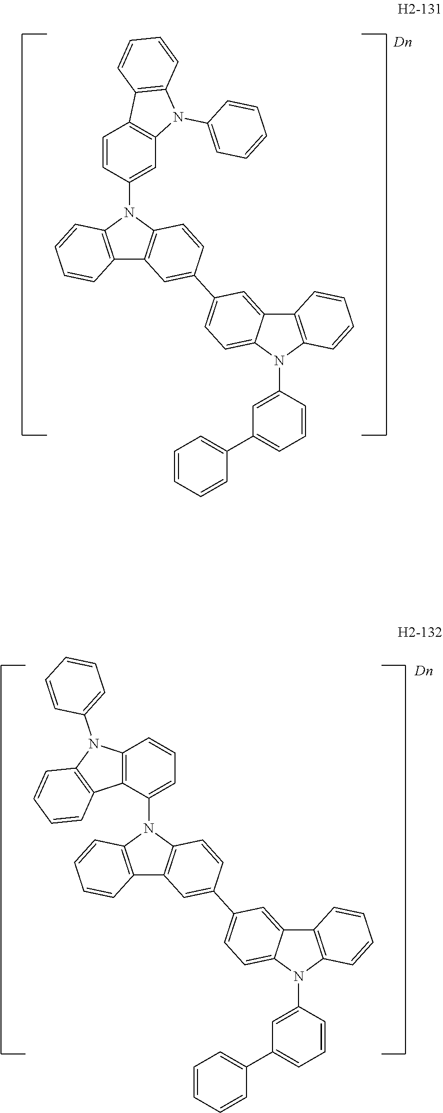 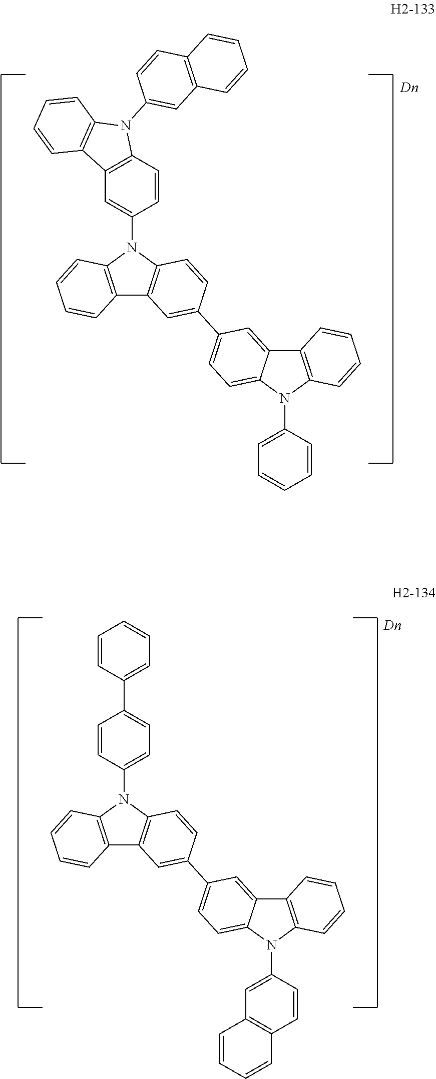 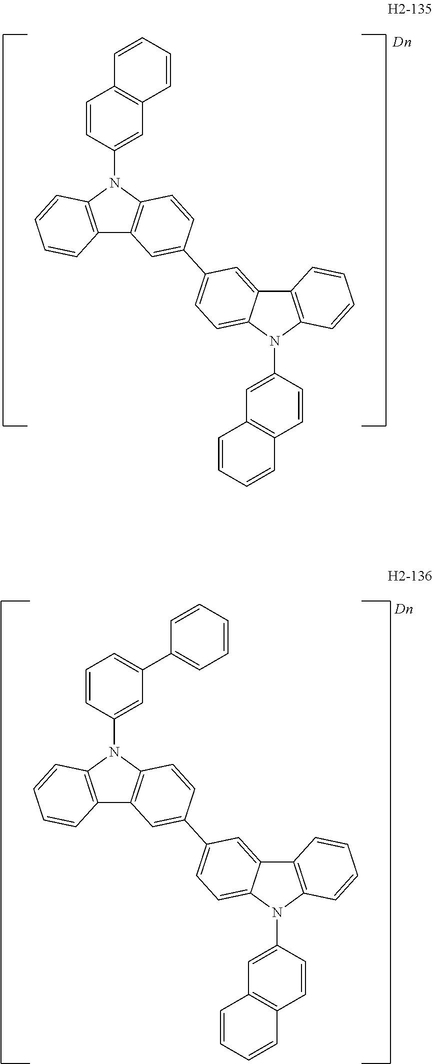 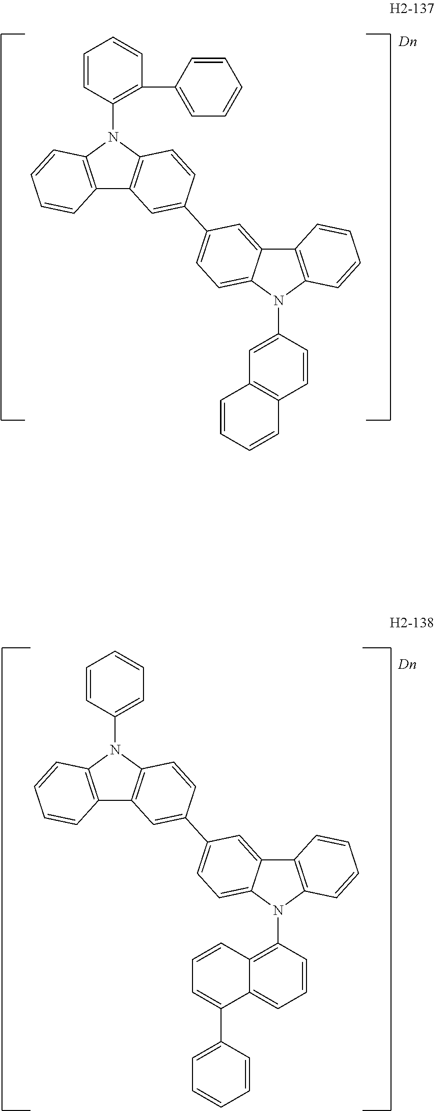 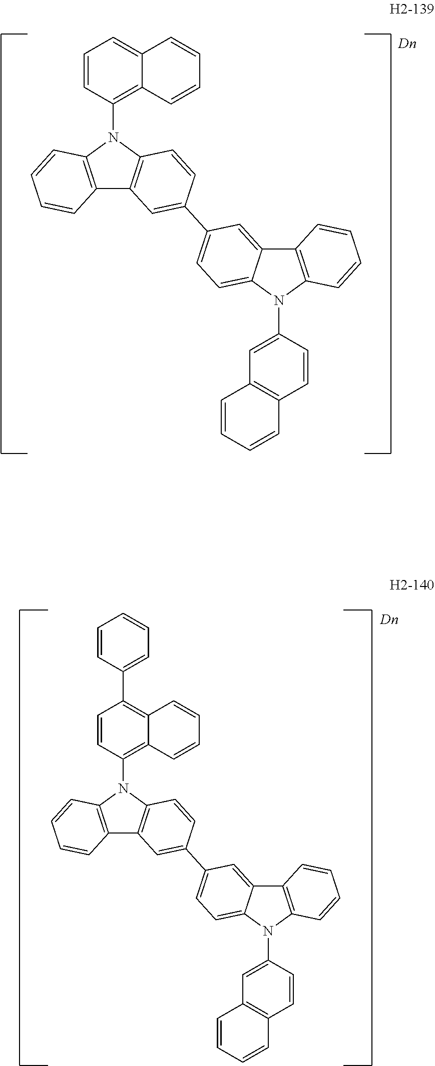 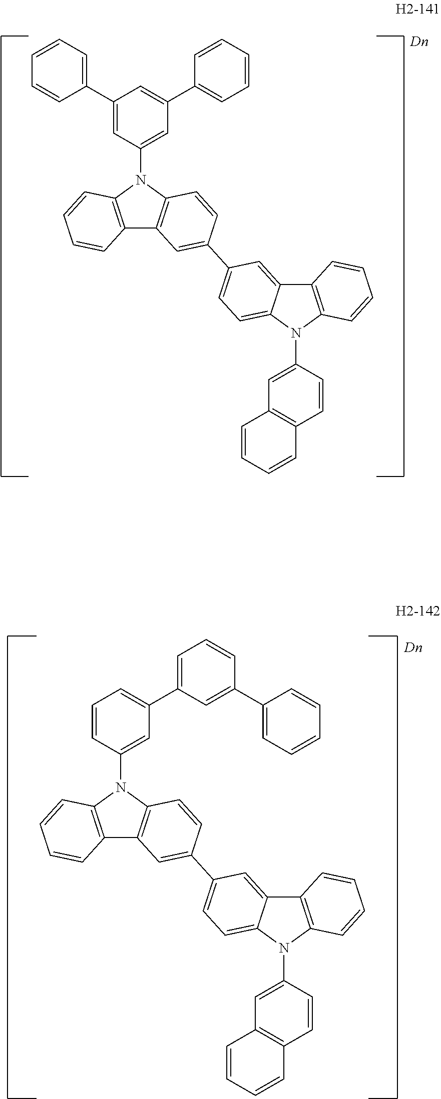 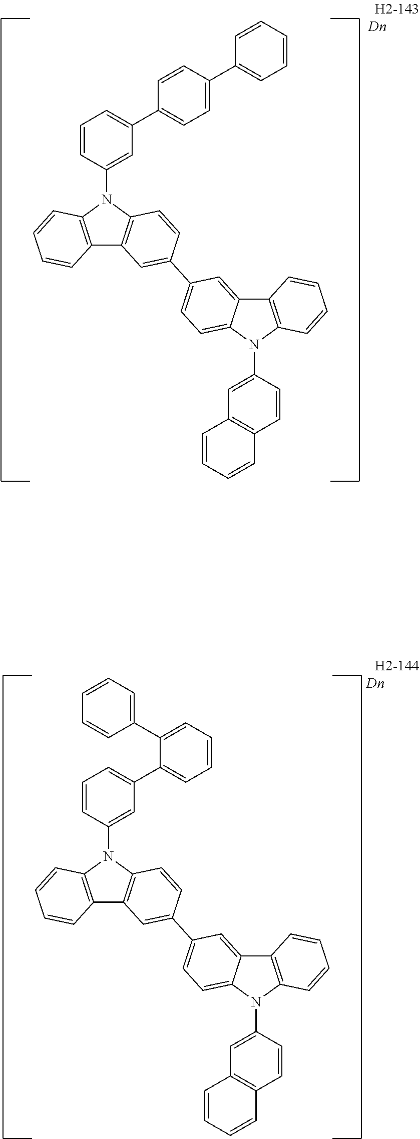 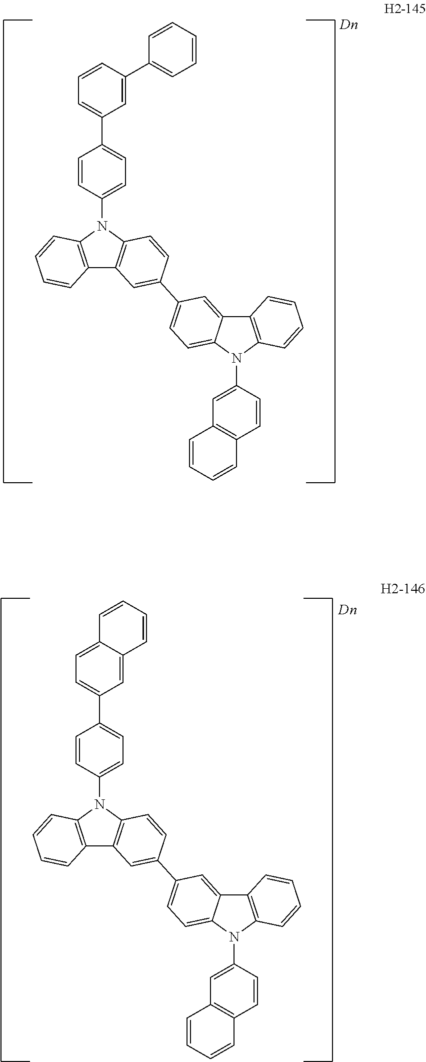 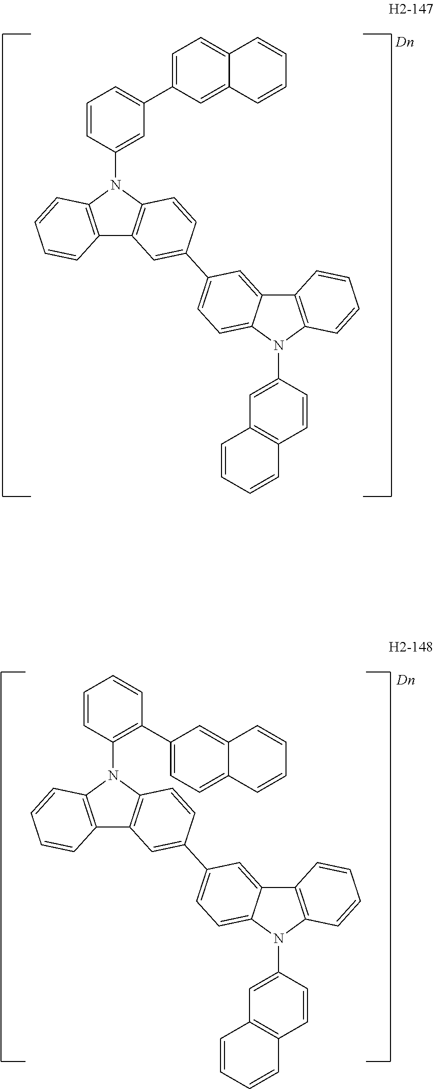 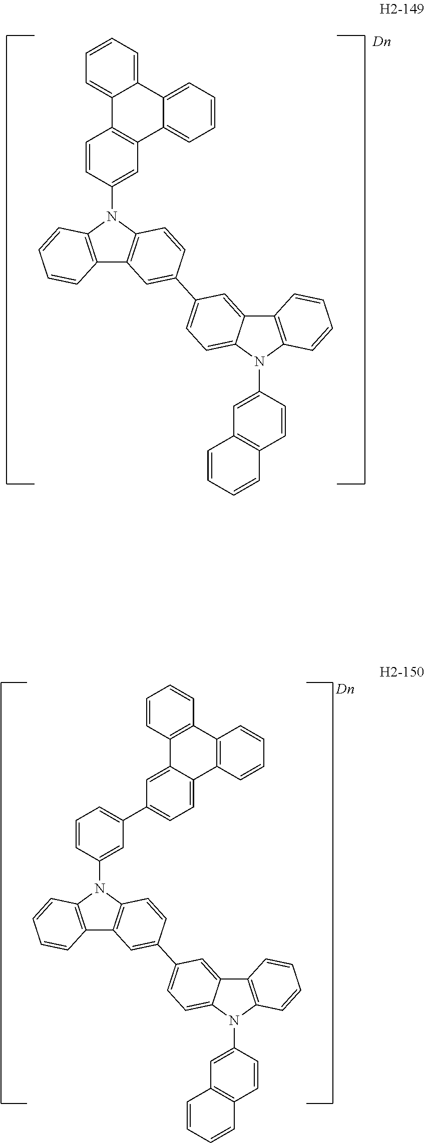 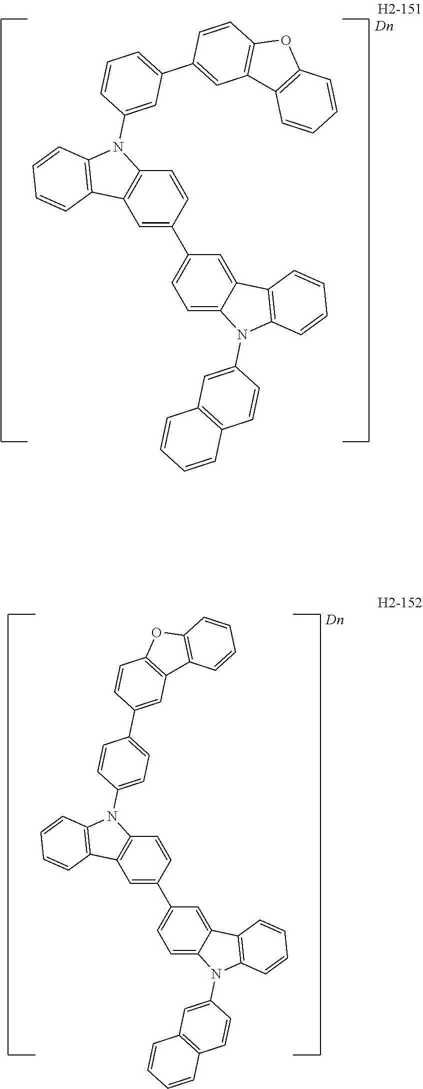 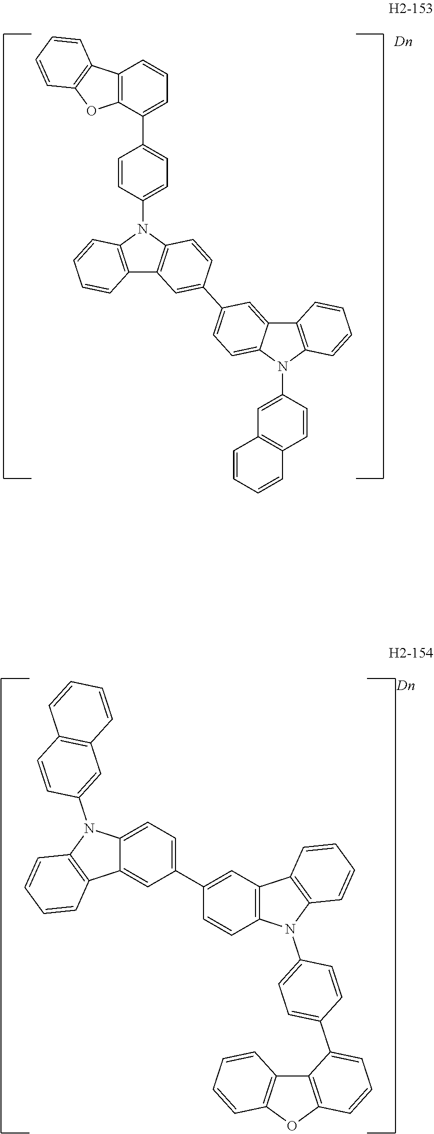 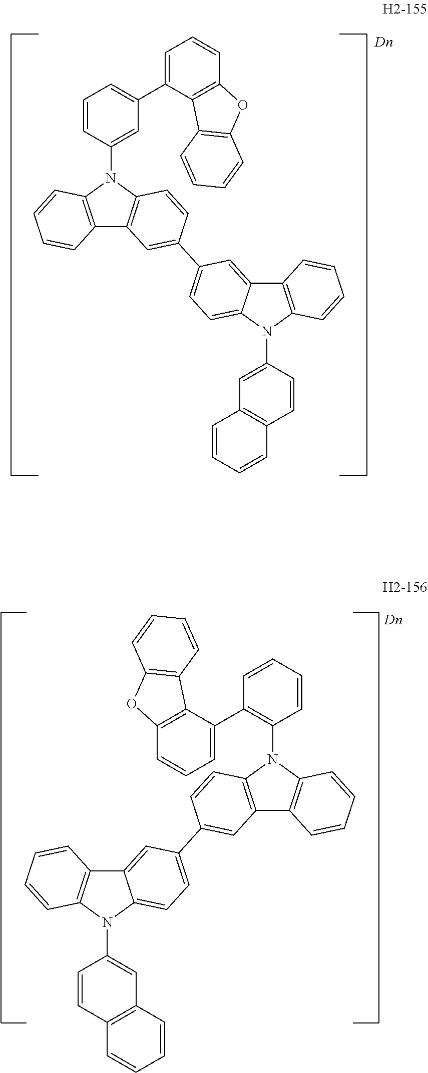 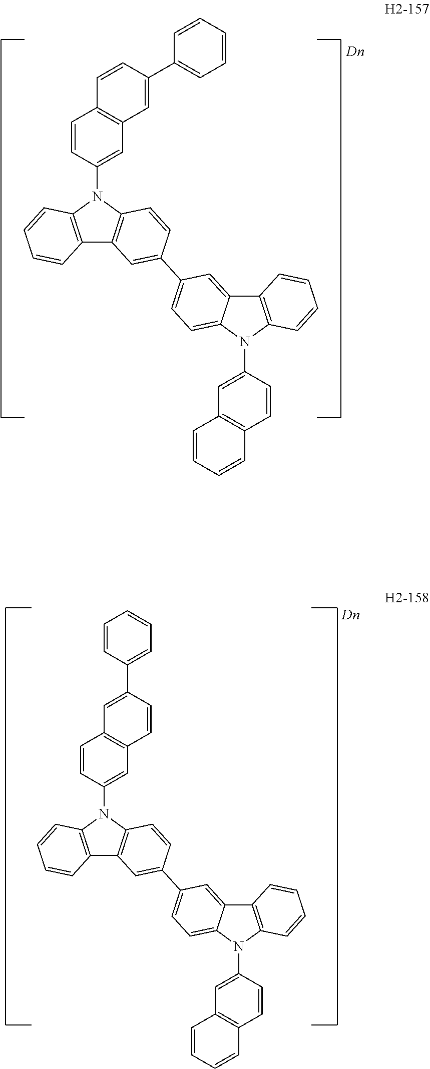 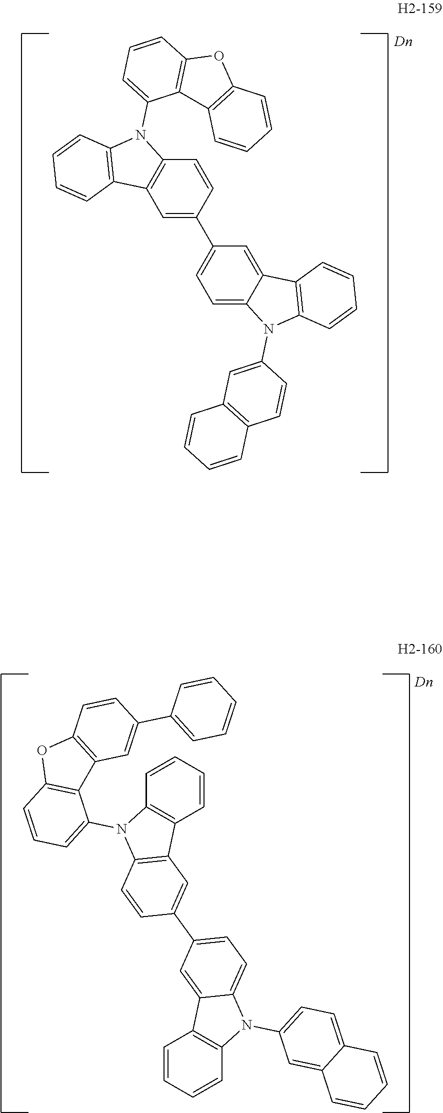 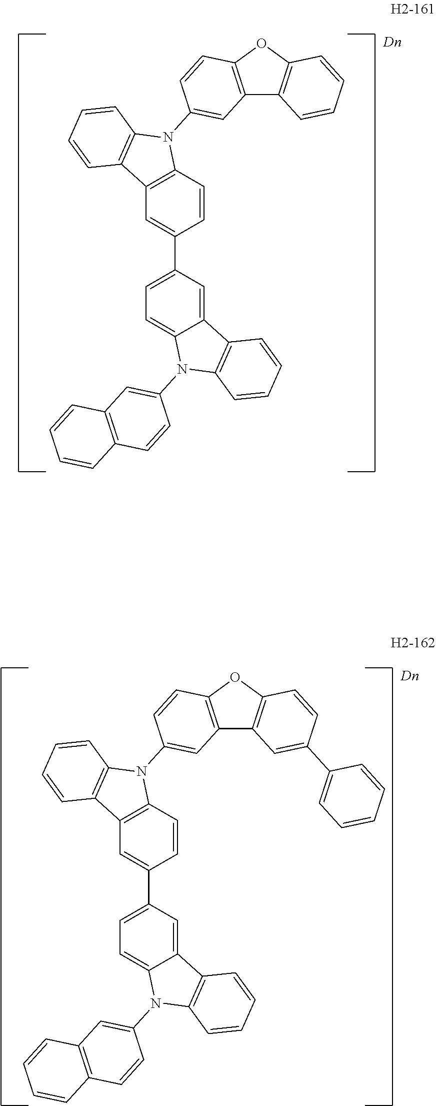 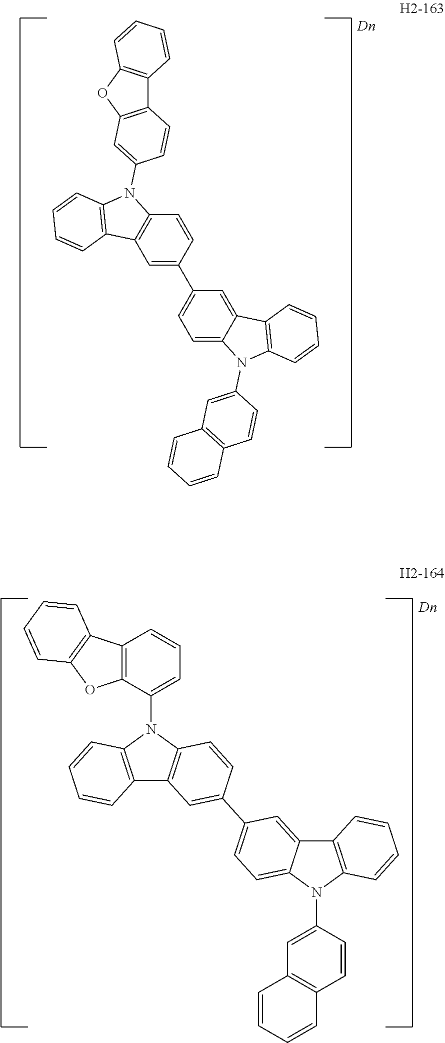 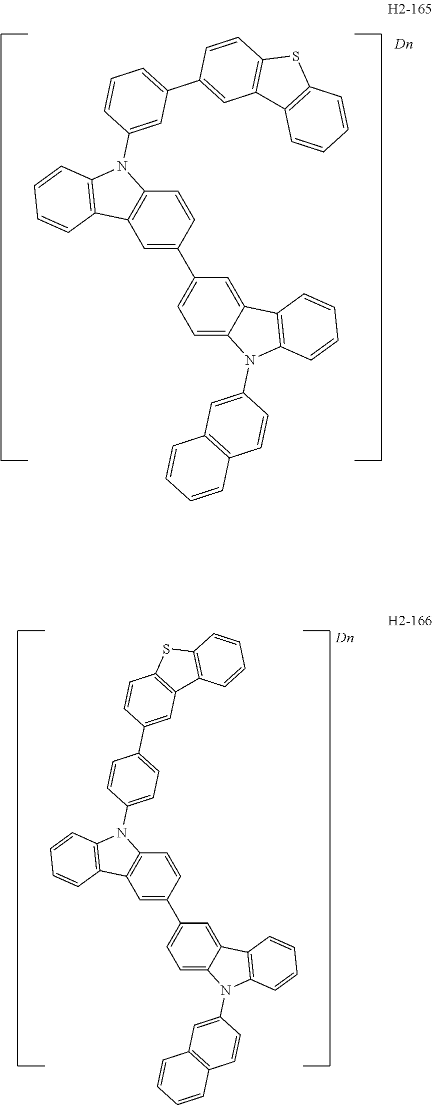 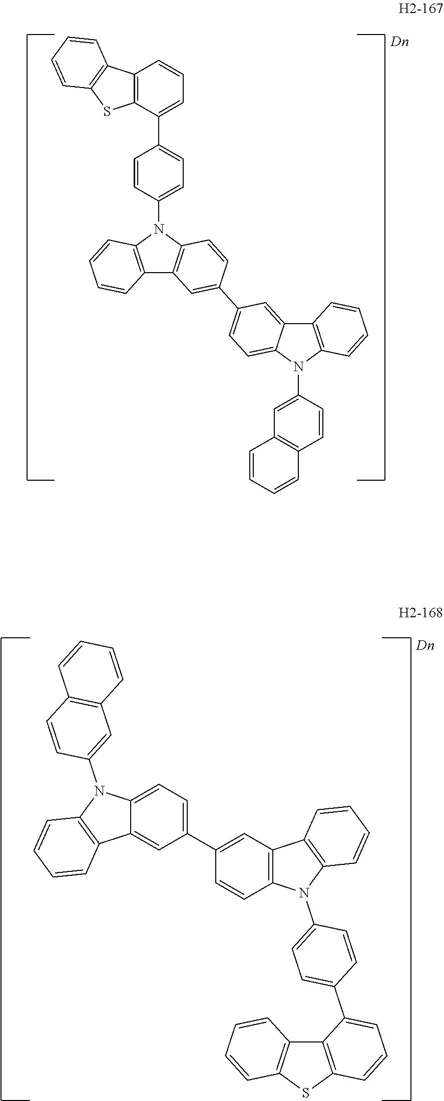 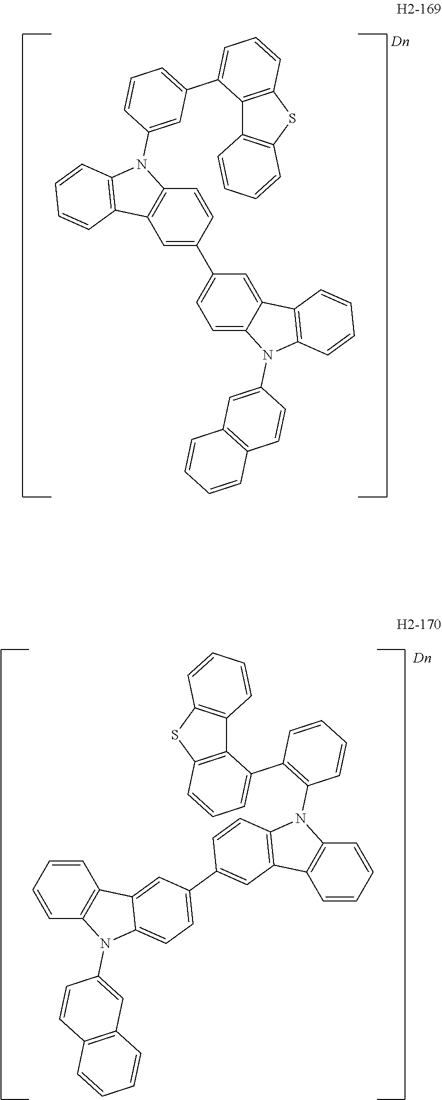 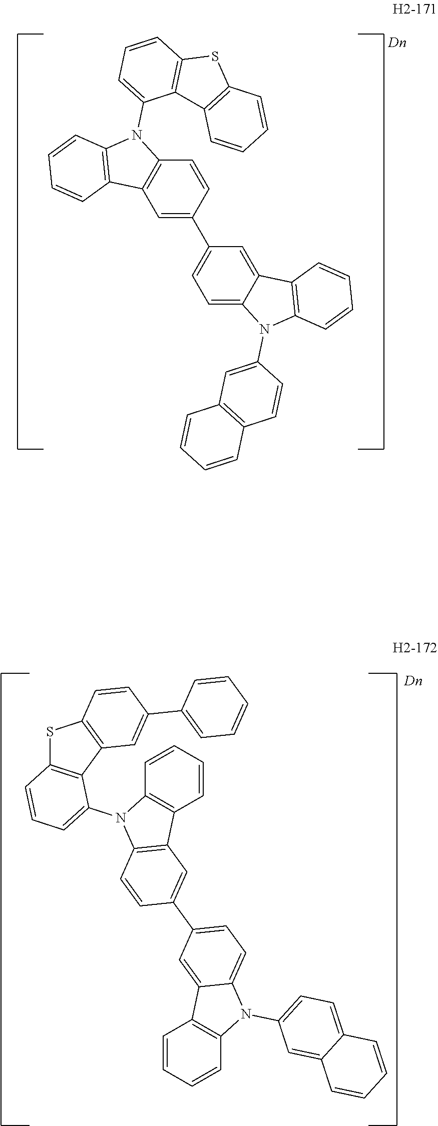 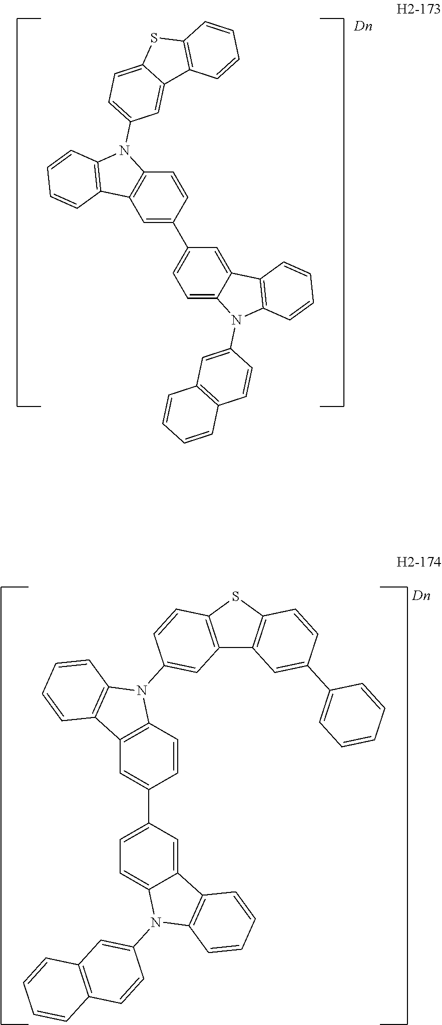 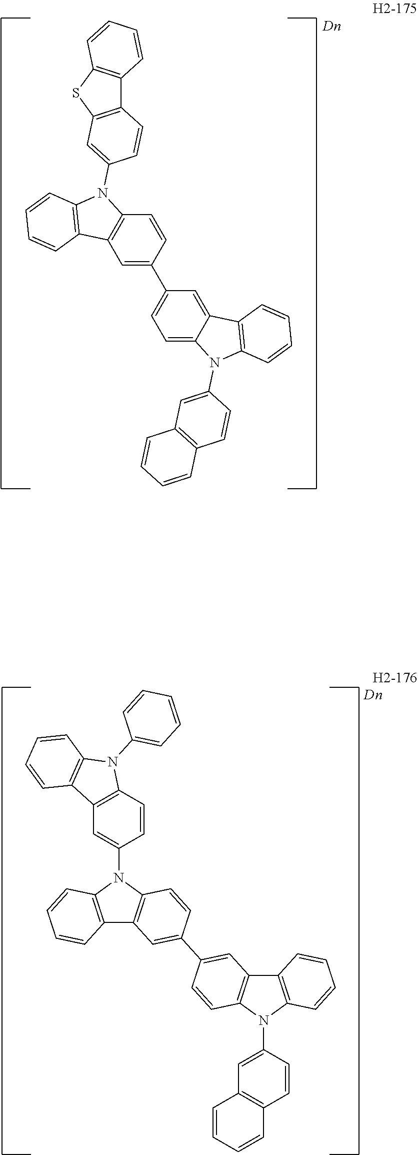 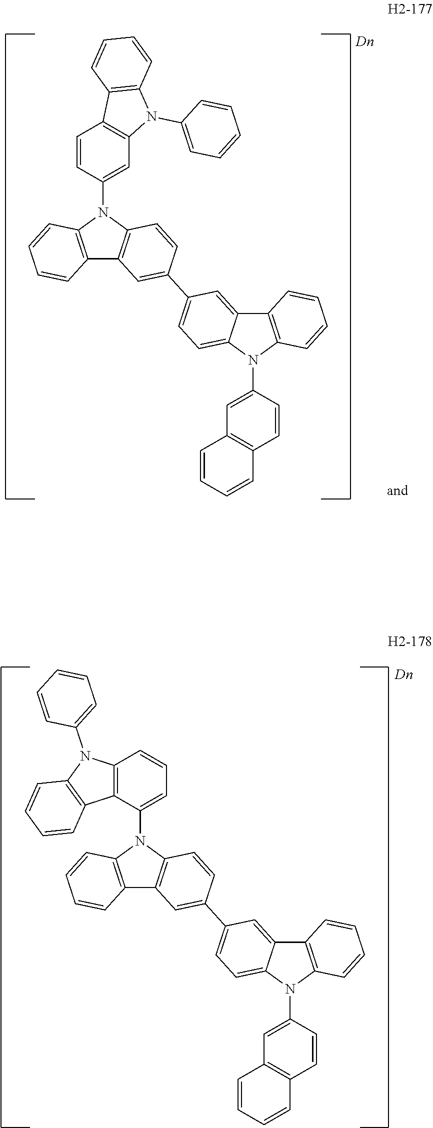
wherein, Dₙ represents that n number of hydrogens are replaced with deuterium; and n represents an integer of 4 to 50.

15. An organic electroluminescent compound represented by the following formula 1′: 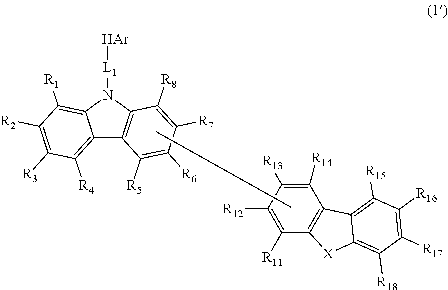
wherein,
L₁ represents a single bond, a substituted or unsubstituted (C1-C30)alkylene, a substituted or unsubstituted (C6-C30)arylene, or a substituted or unsubstituted (3- to 30-membered)heteroarylene;
HAr represents a substituted or unsubstituted nitrogen-containing (3- to 30-membered)heteroaryl;
R₁ to R₈, and R₁₁ to R₁₈, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl,
any one of R₅ to R₈ is linked to any one of R₁₁ to R₁₄ to form a single bond;
X represents O, S, CR₂₁R₂₂, SiR₂₃R₂₄, or NR₂₅; and
R₂₁ to R₂₅, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl, or may be linked to an adjacent substituent(s) to form a ring(s);
with the proviso that at least five of R₁ to R₈, and R₁₁ to R₁₈ are deuterium.

16. The organic electroluminescent compound according to claim 15, wherein the compound represented by formula 1′ is selected from the following compounds: 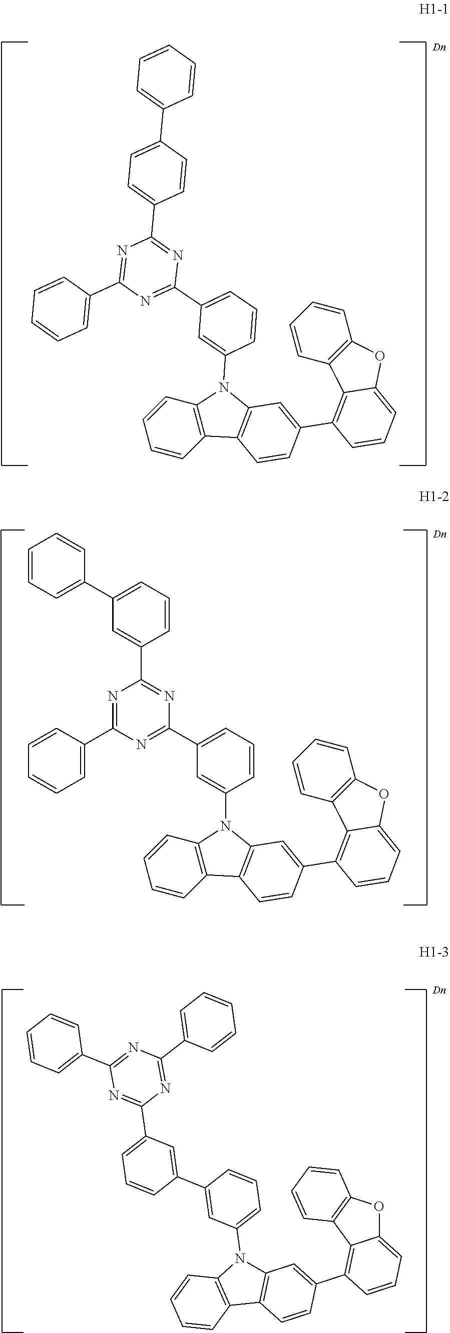 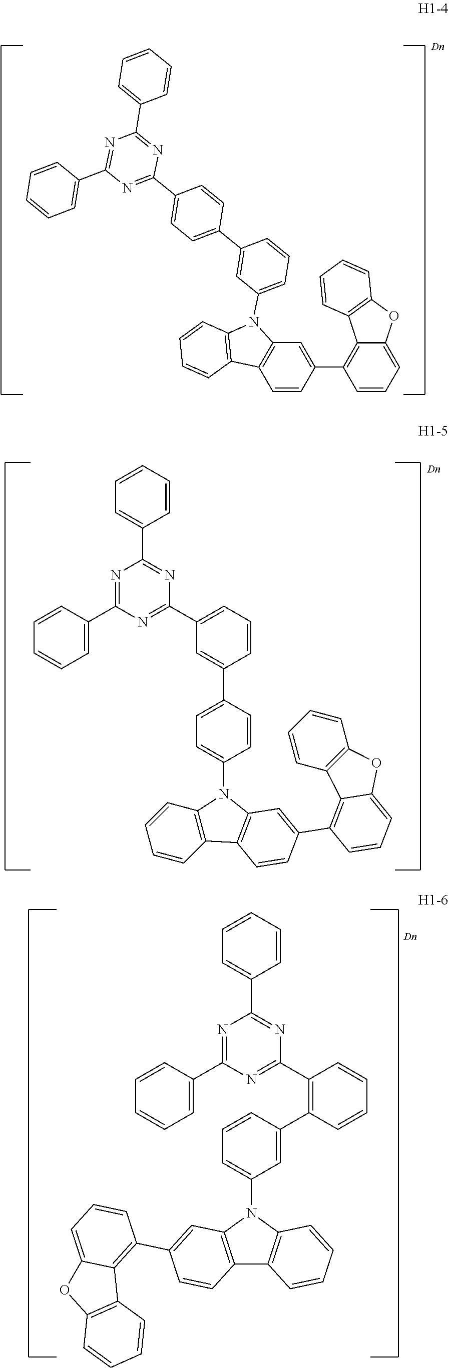 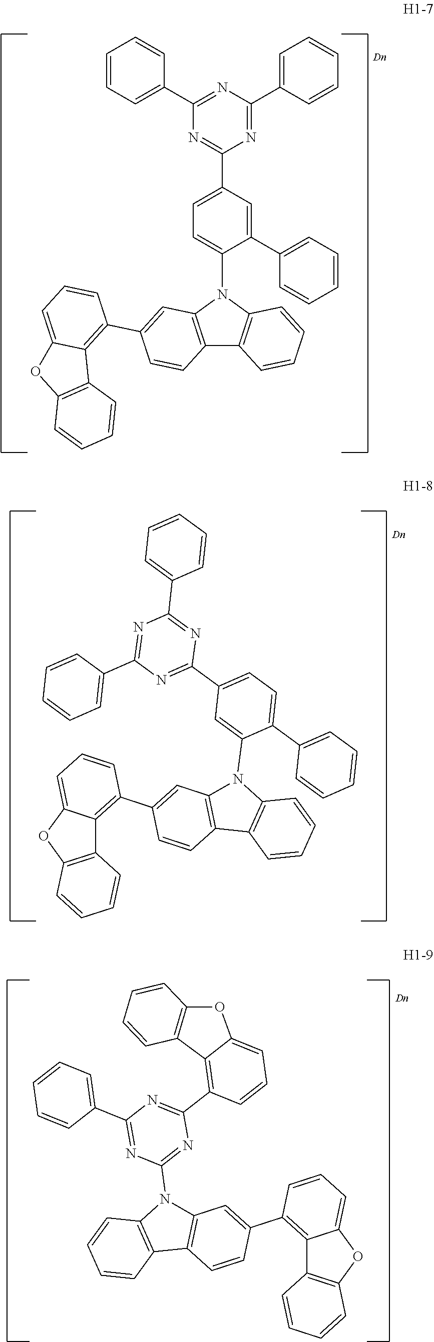 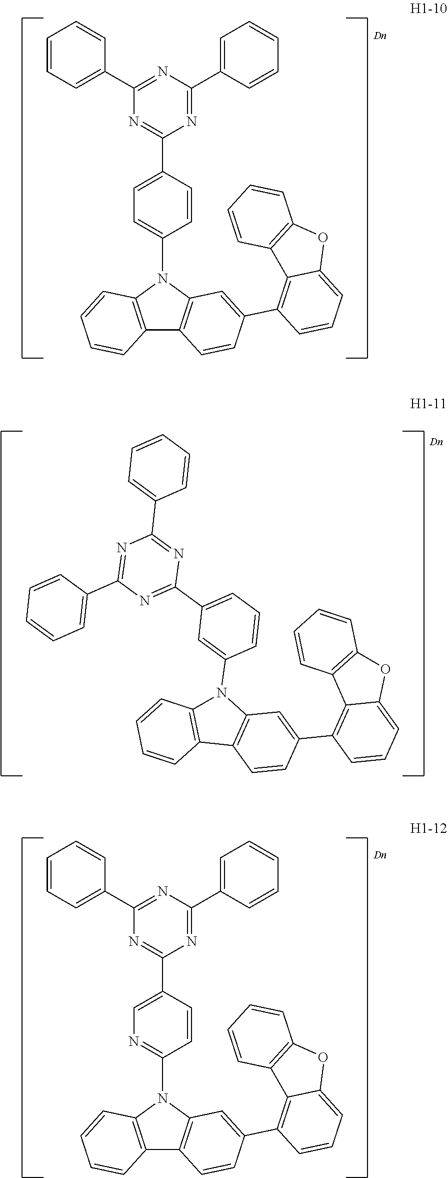 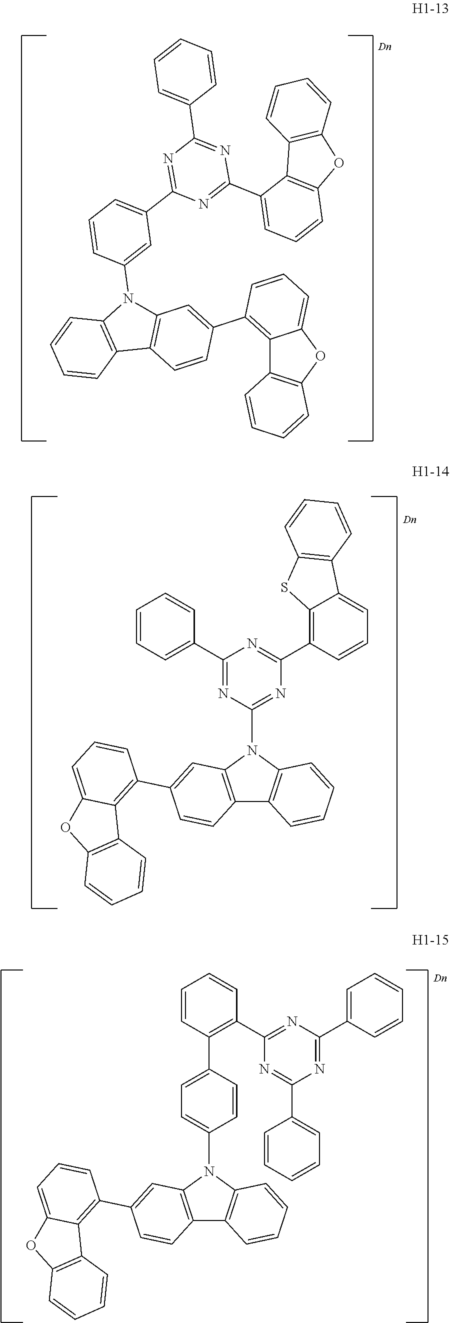 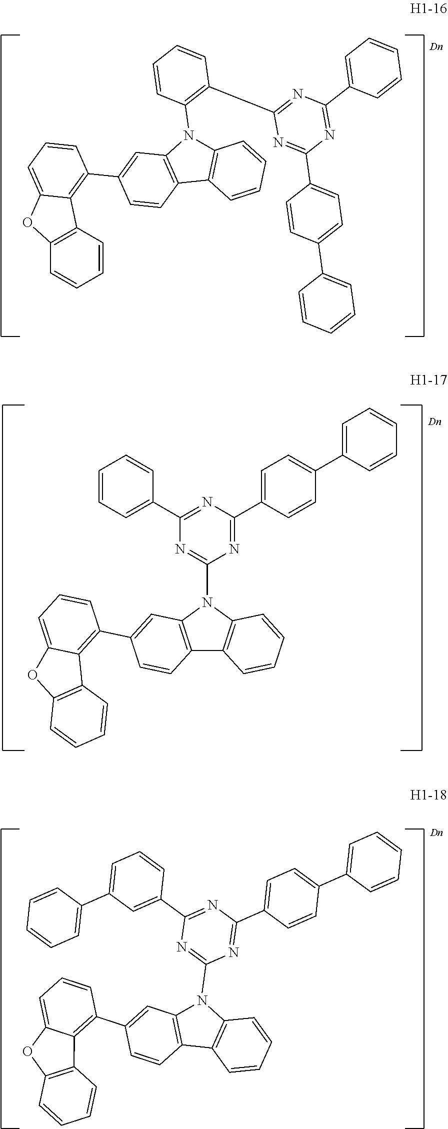 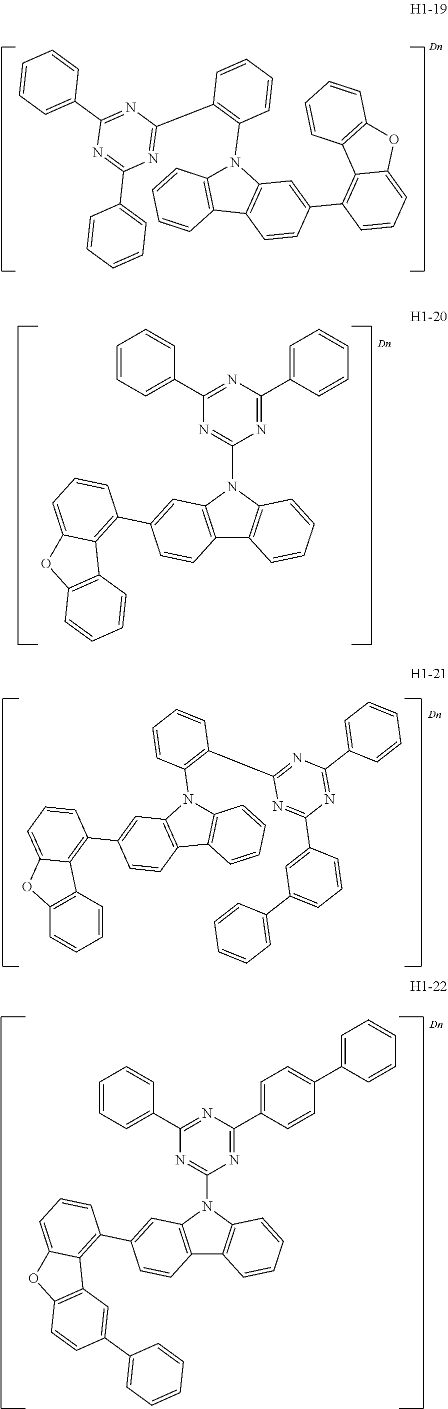 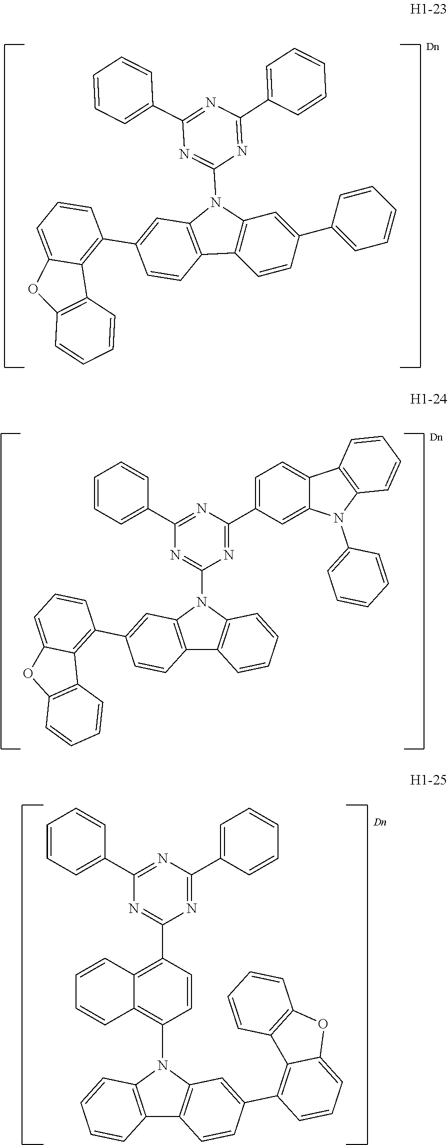 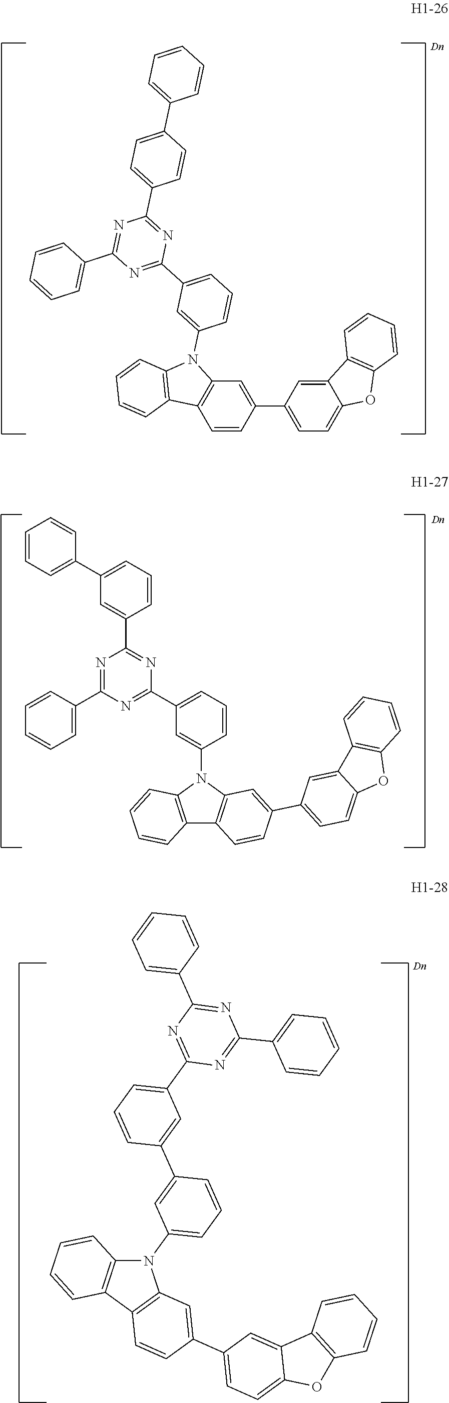 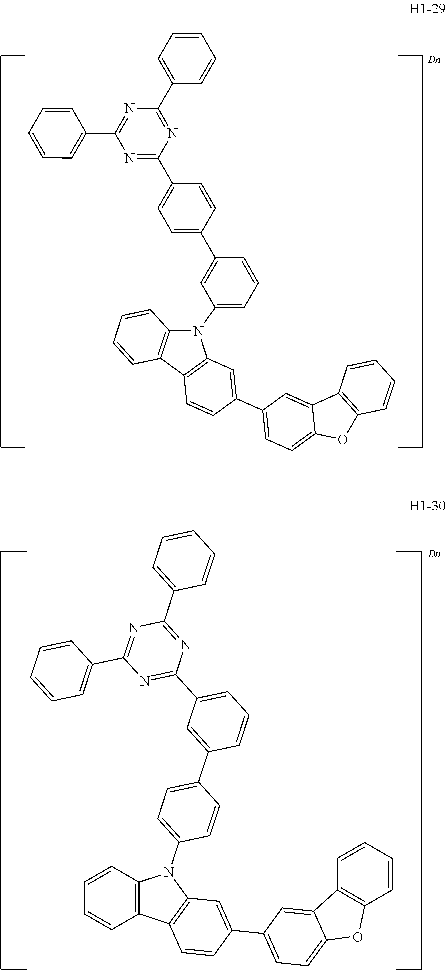 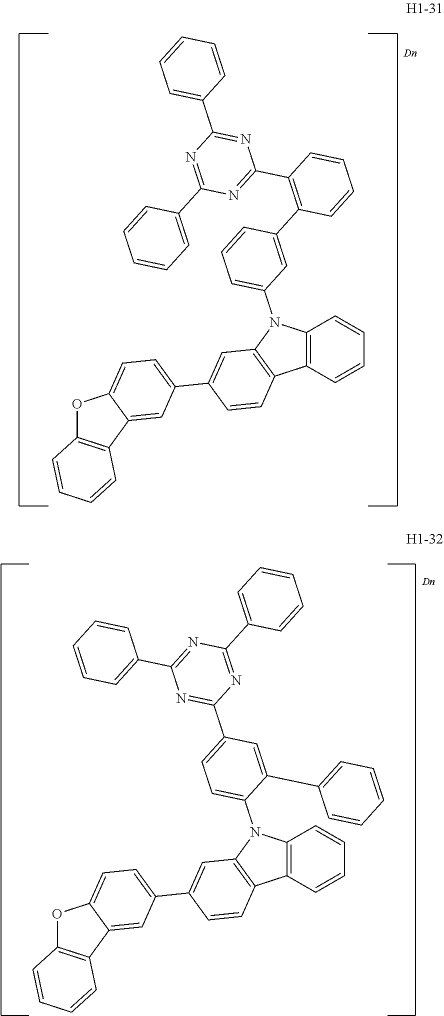 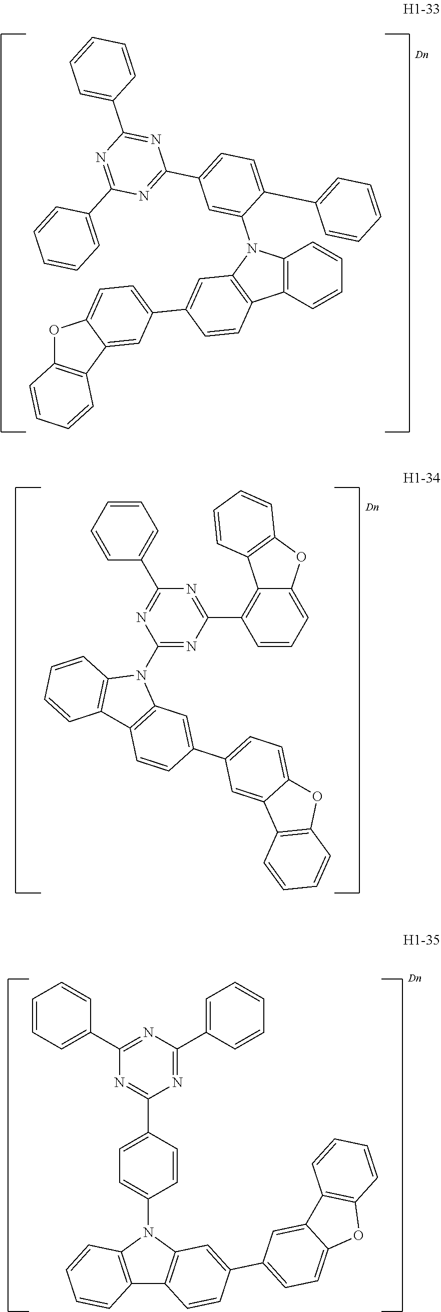 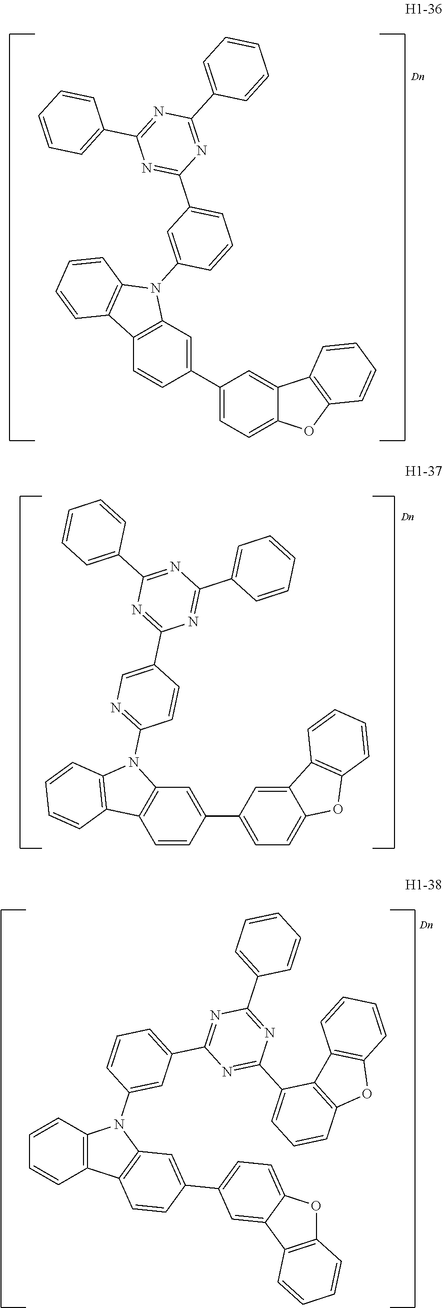 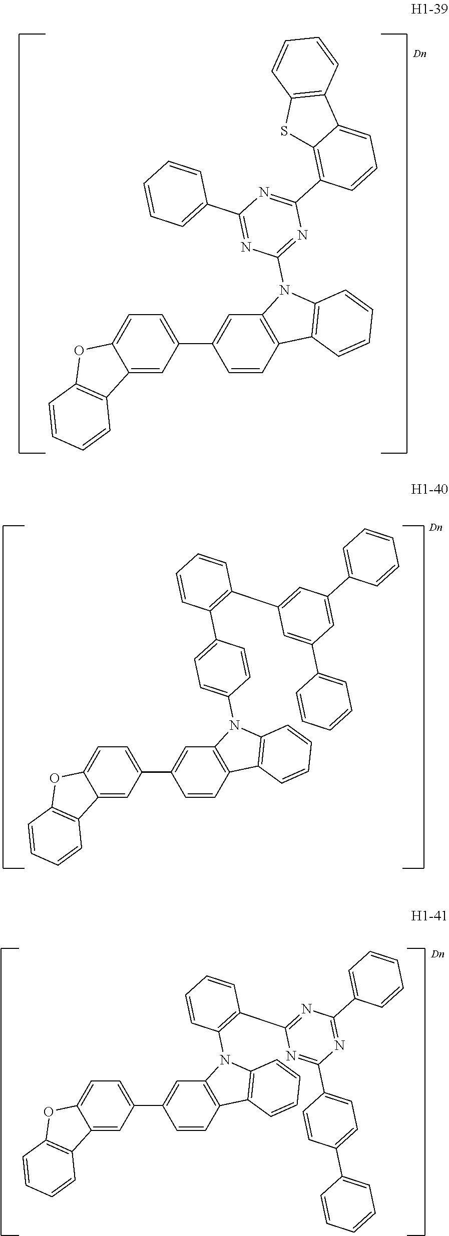 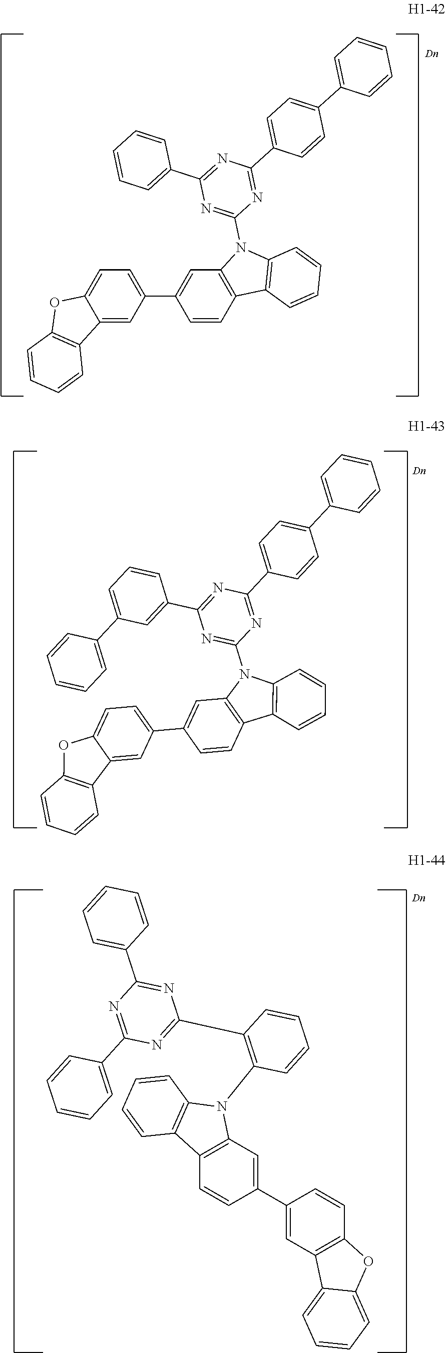 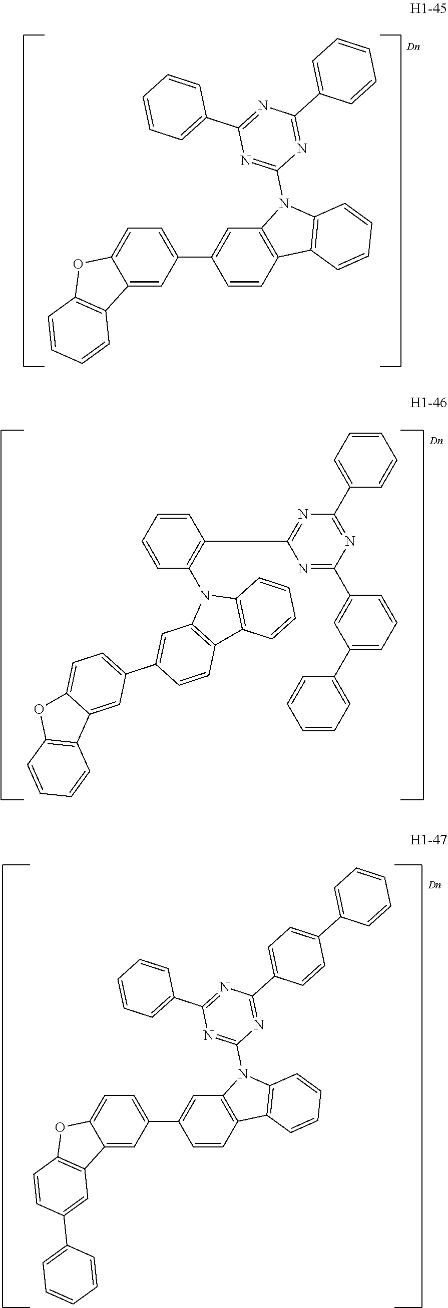 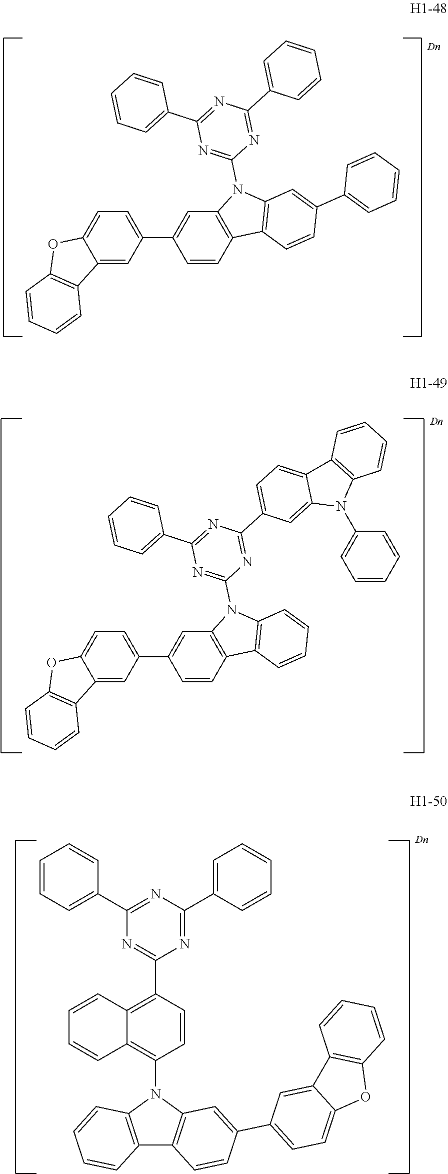 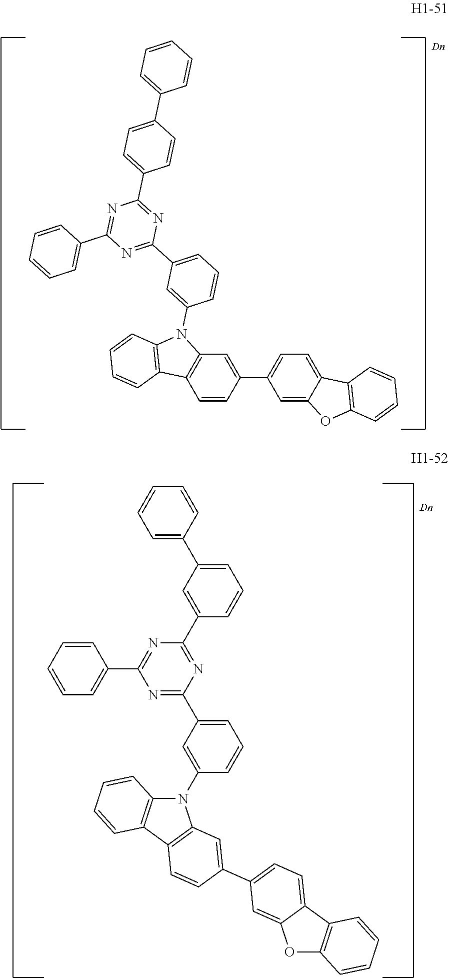 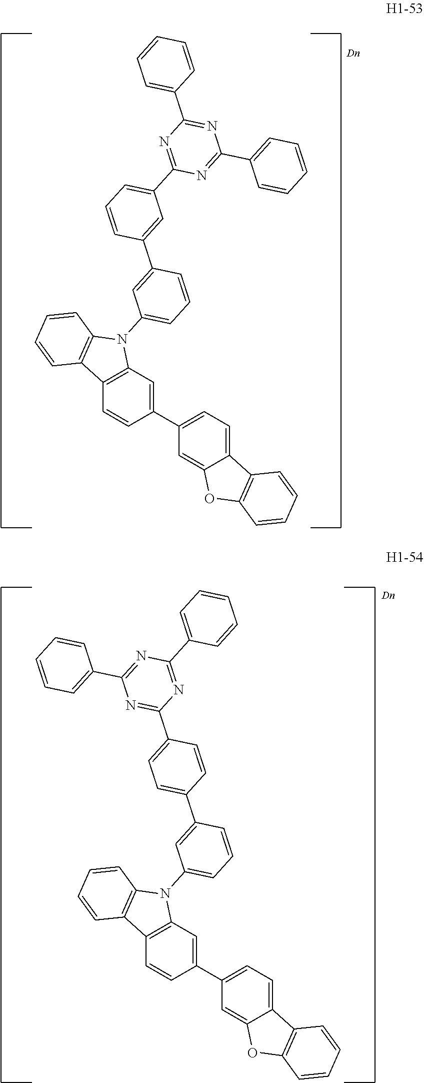 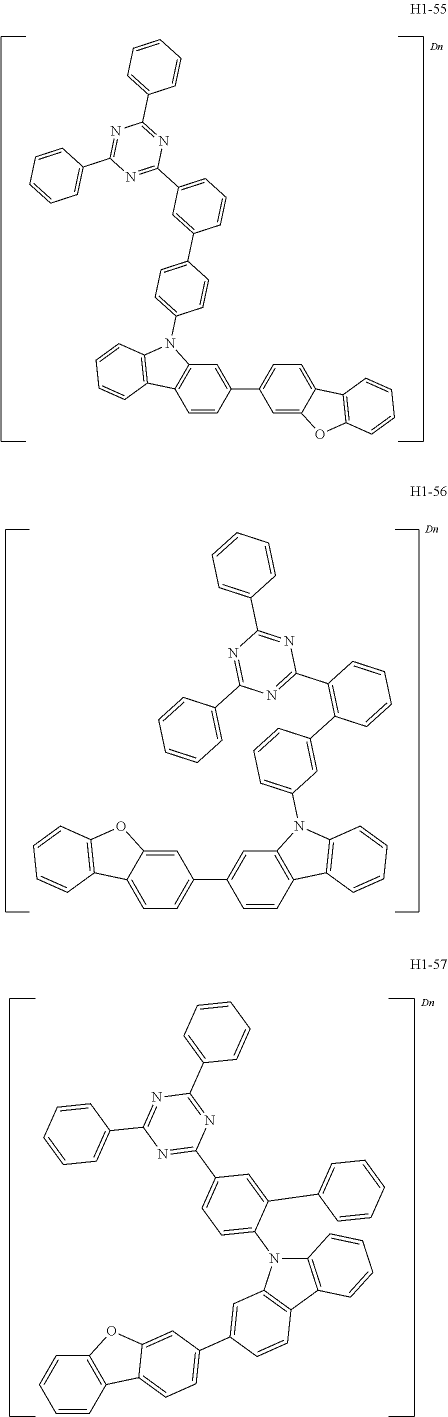 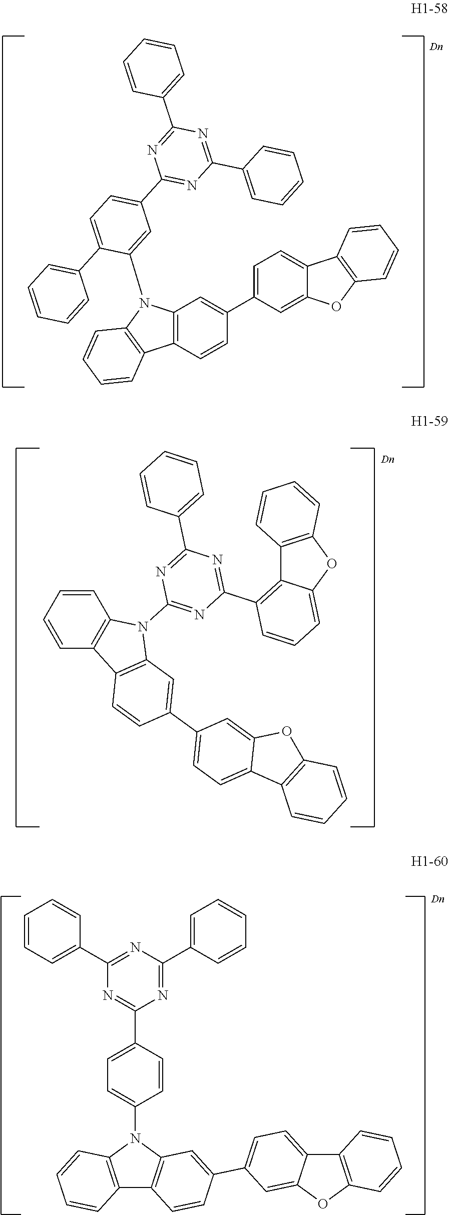 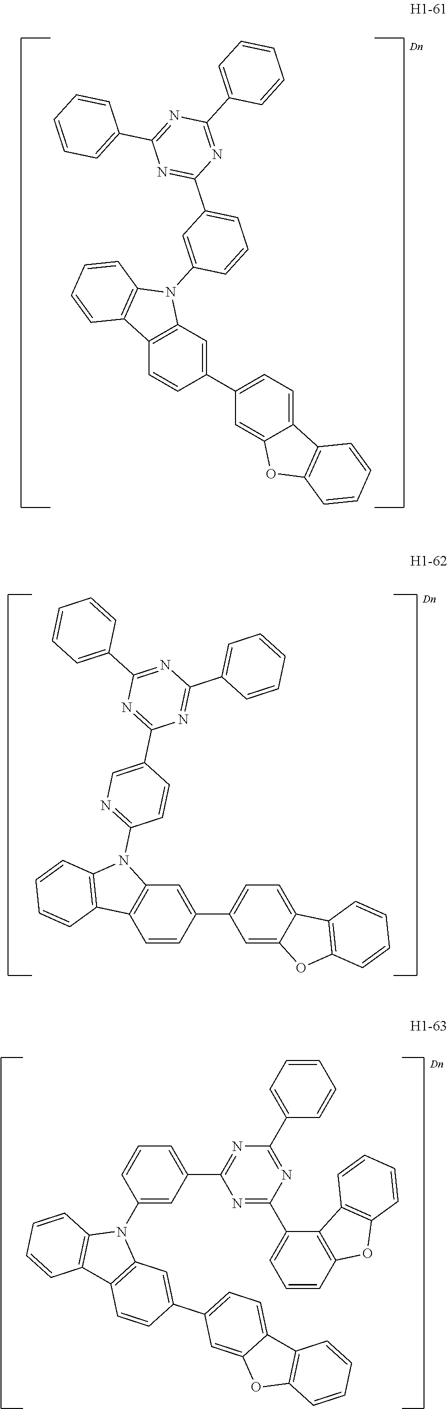 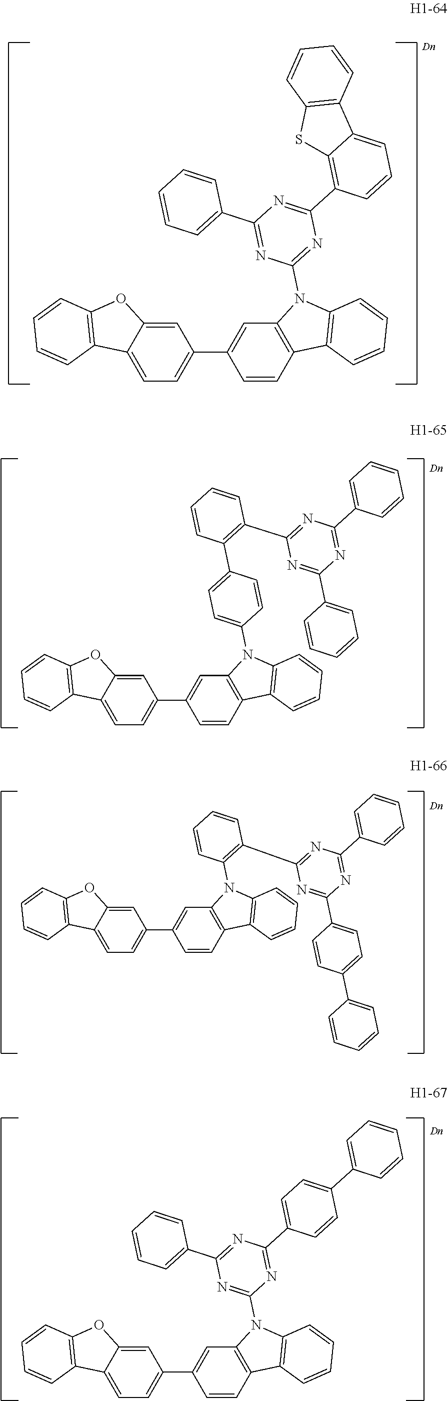 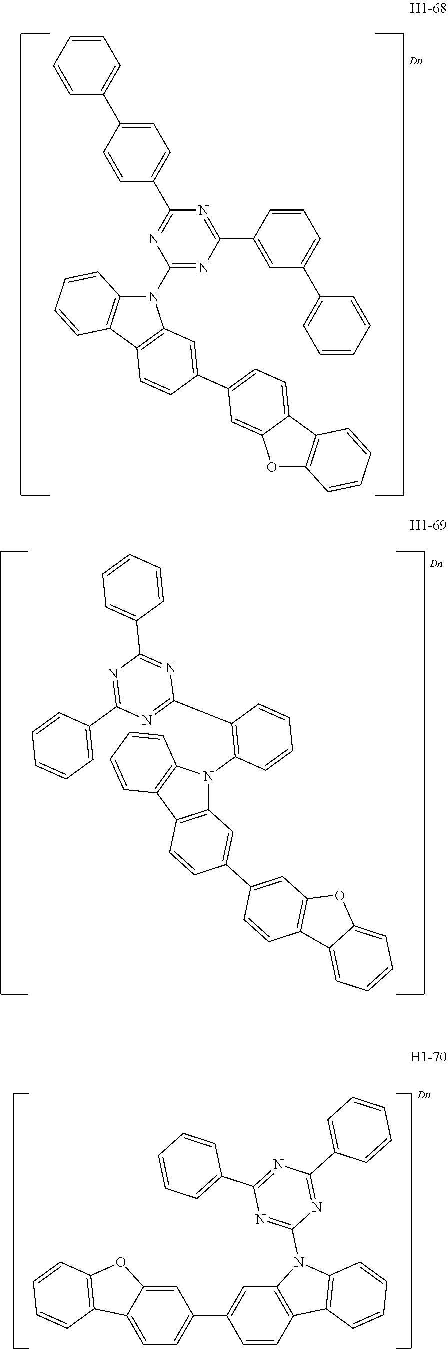 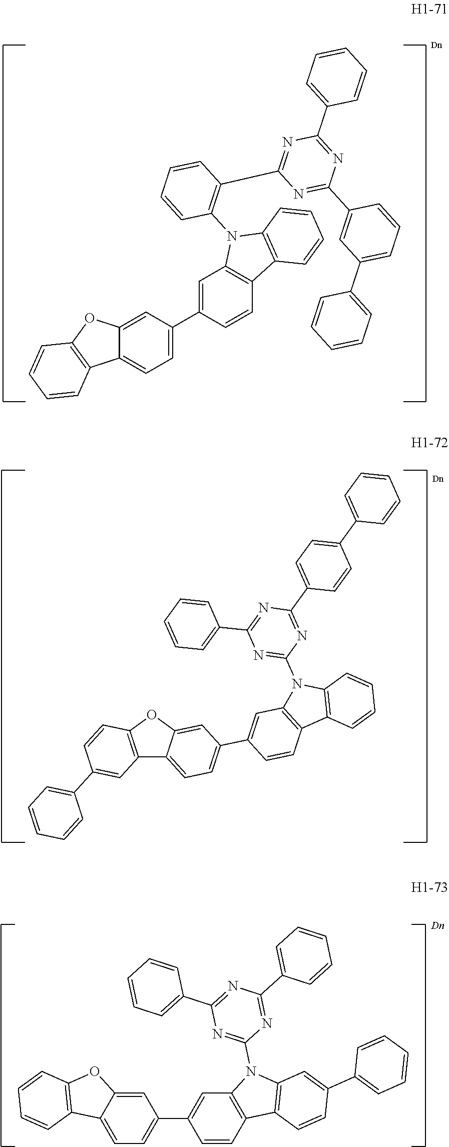 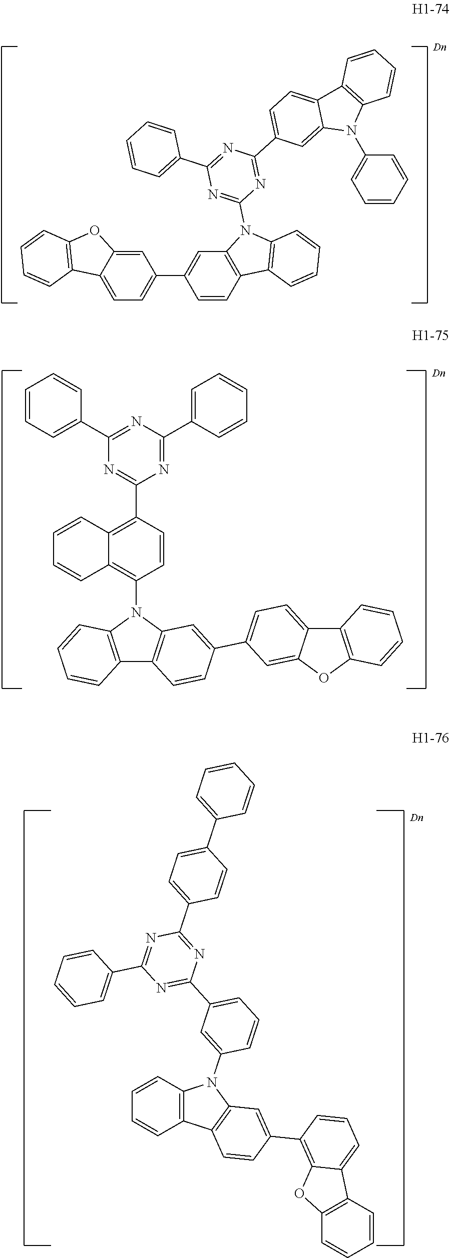 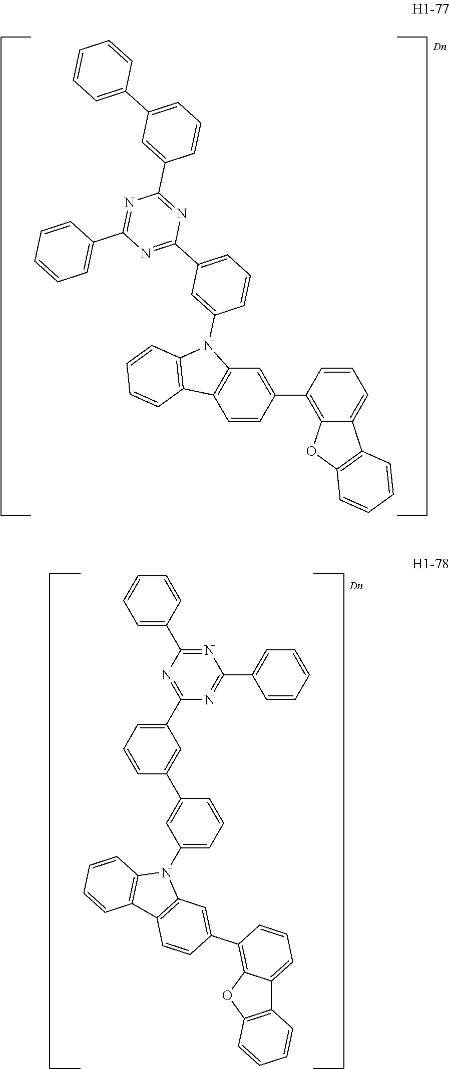 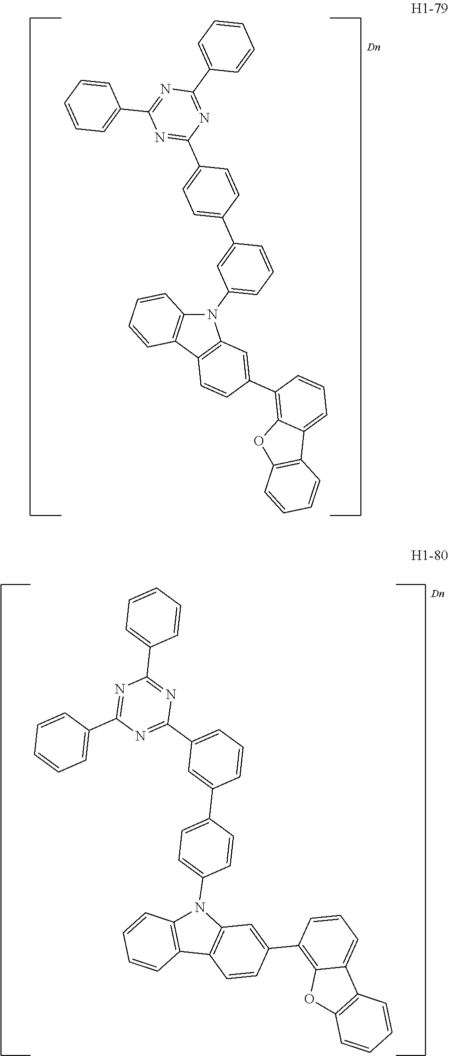 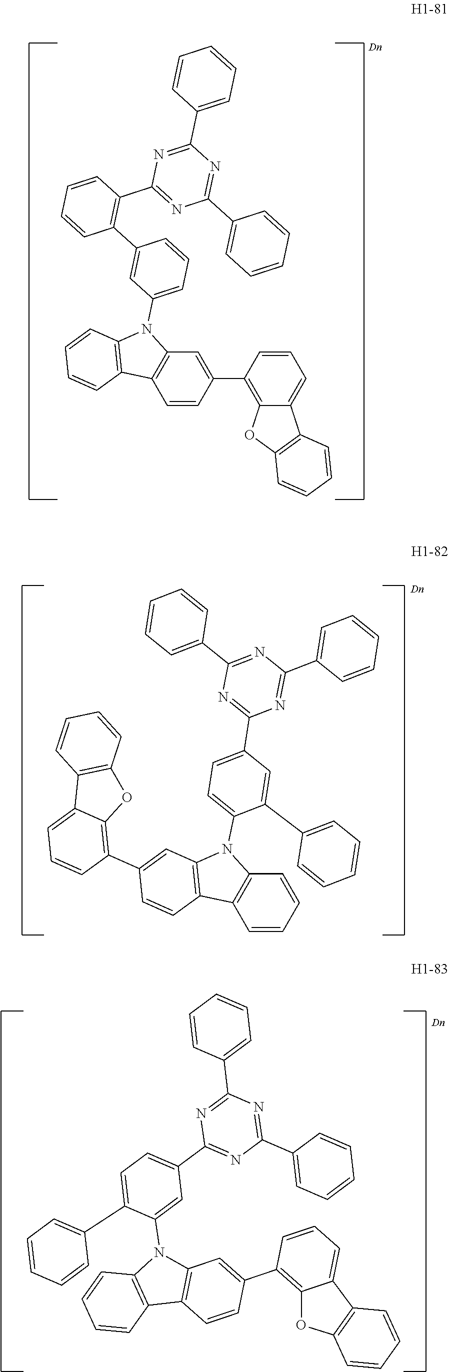 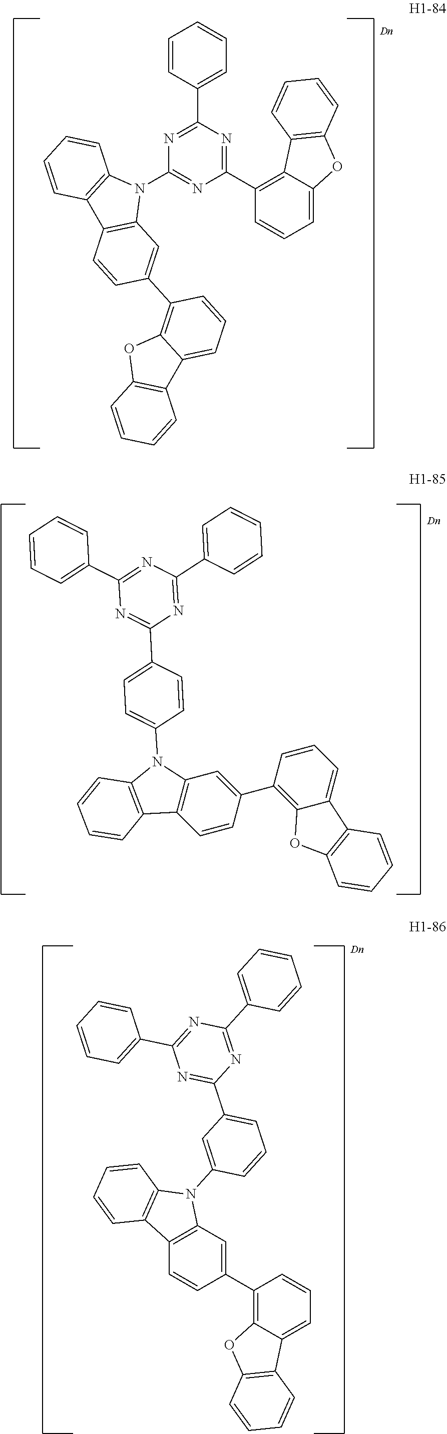 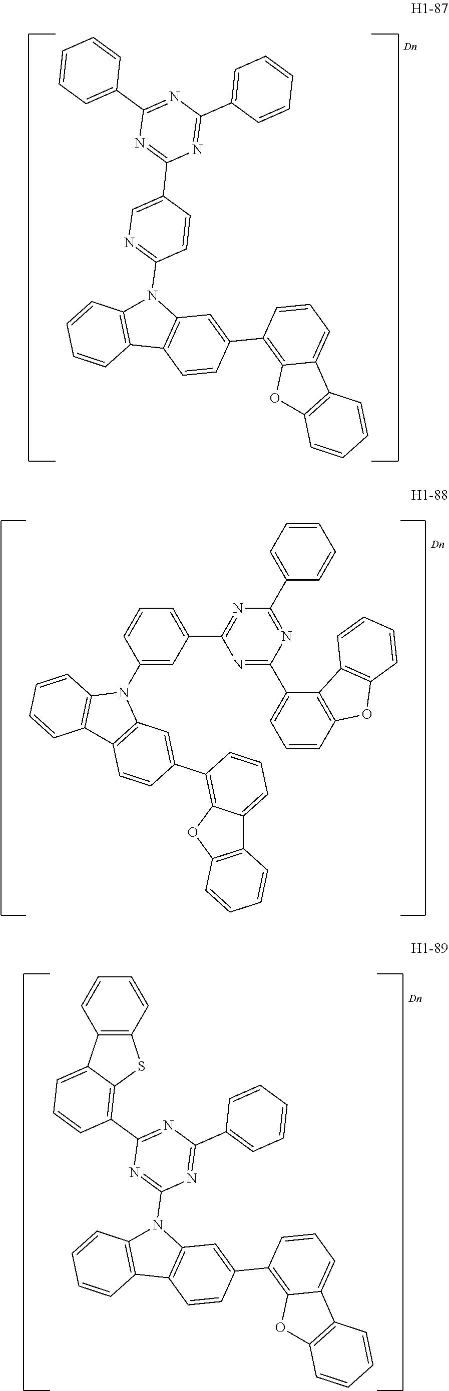 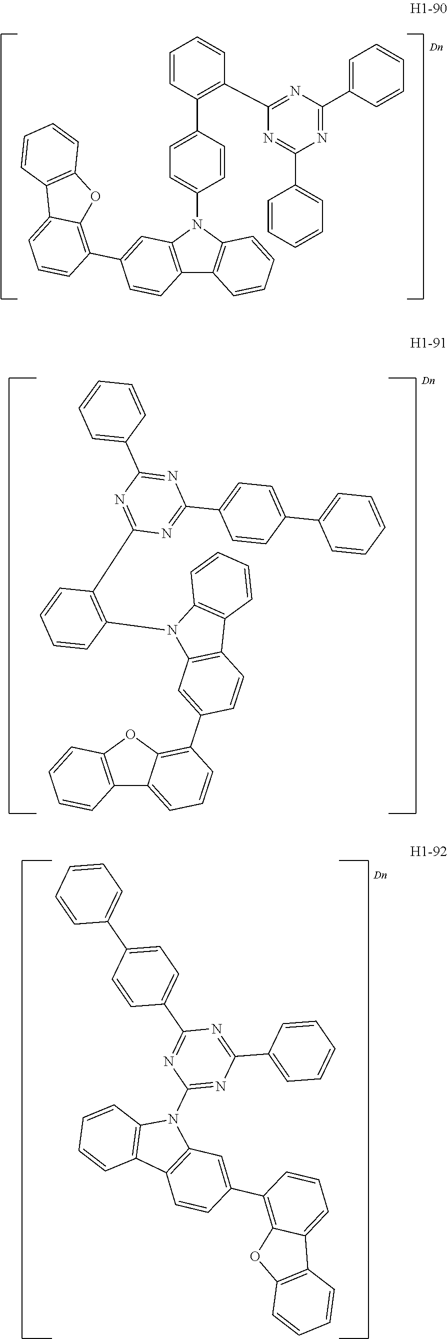 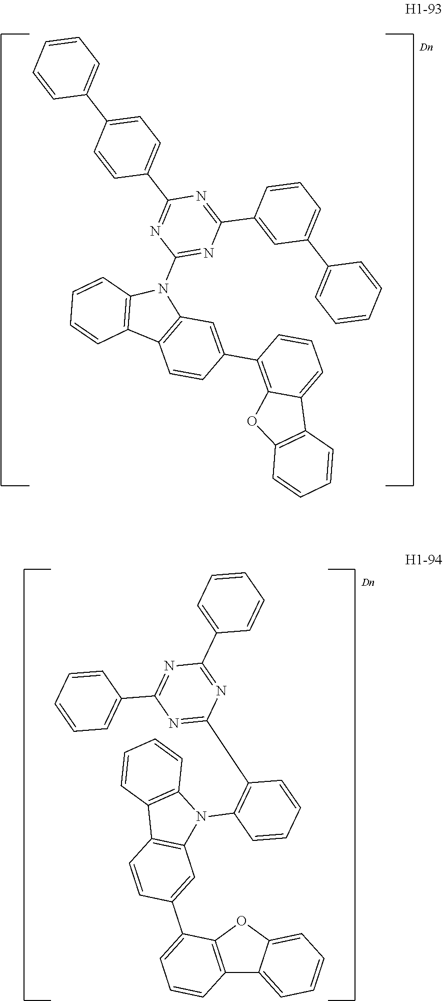 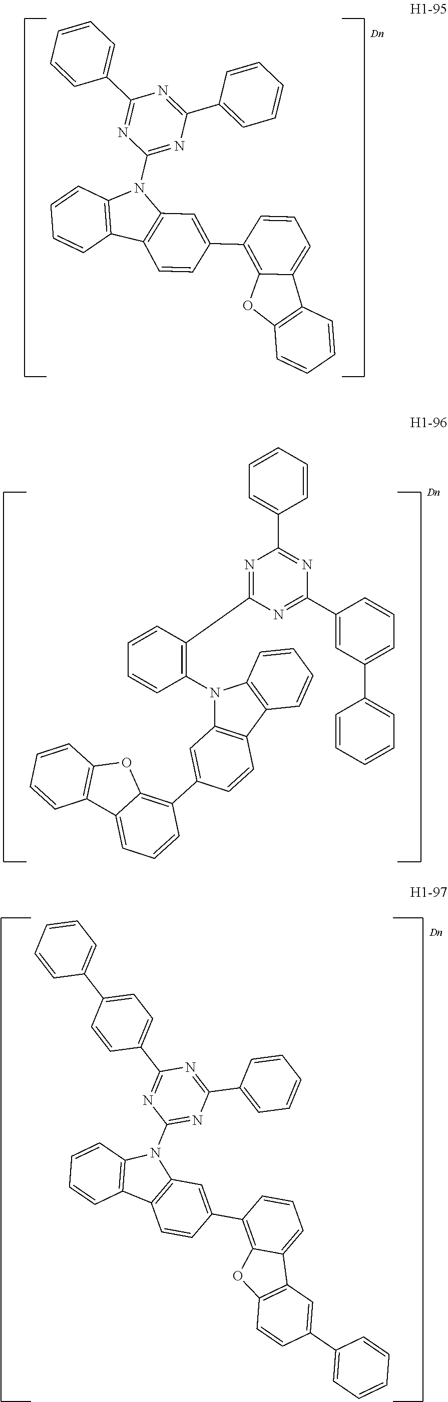 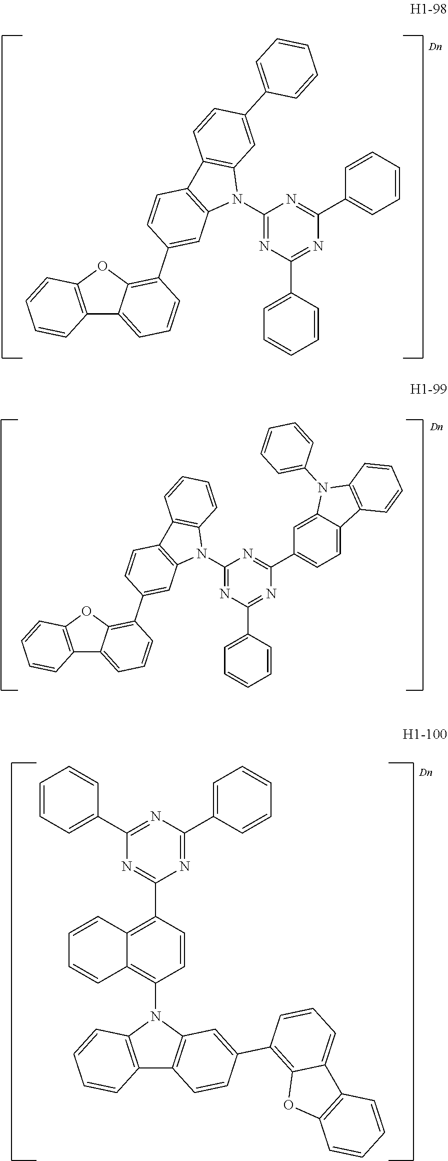 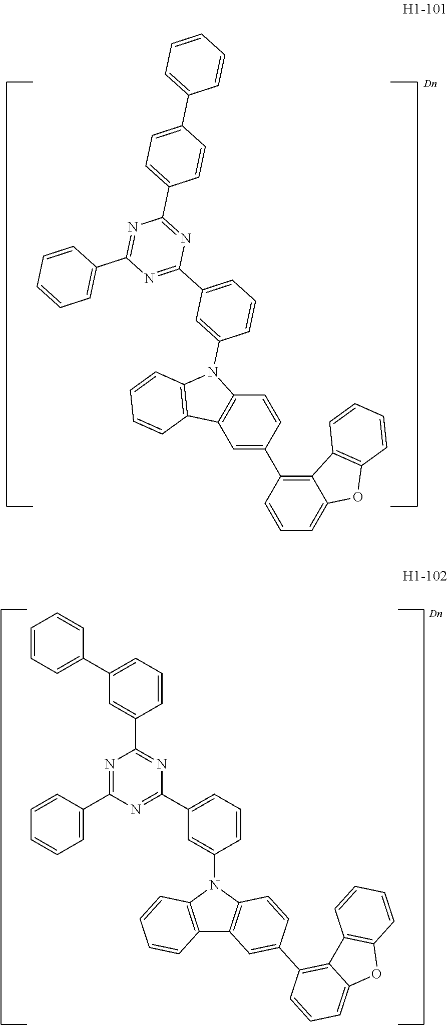 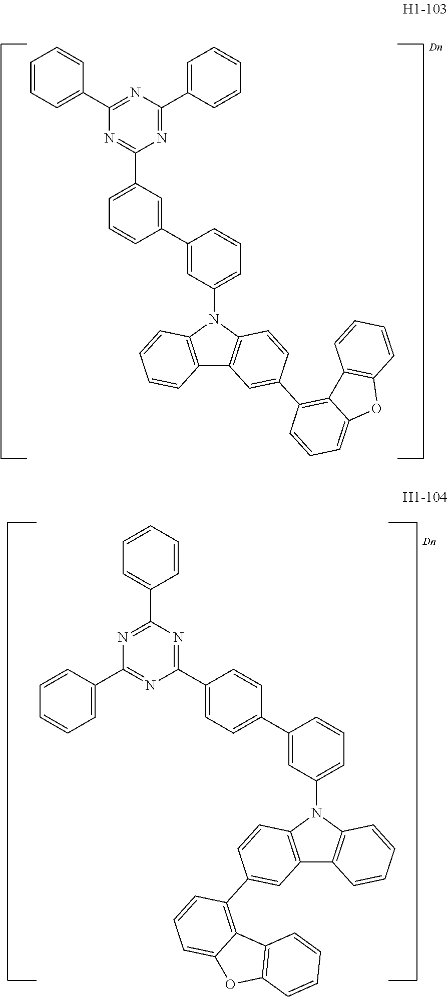 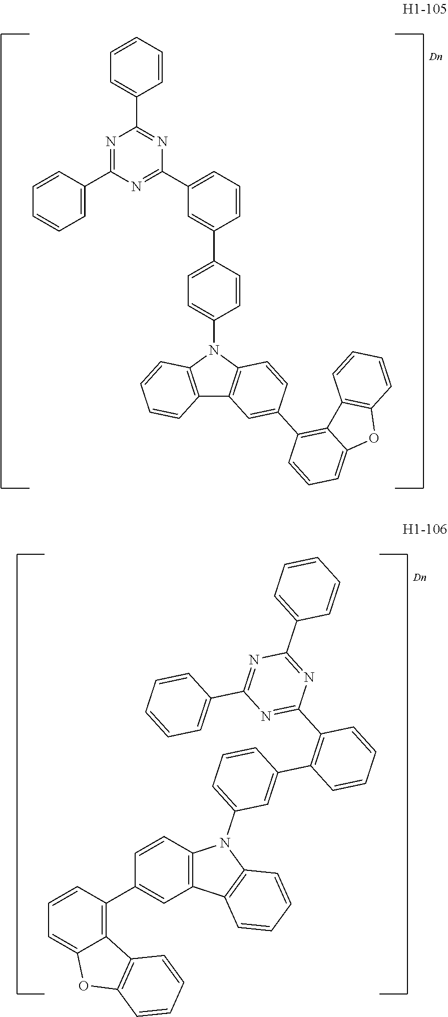 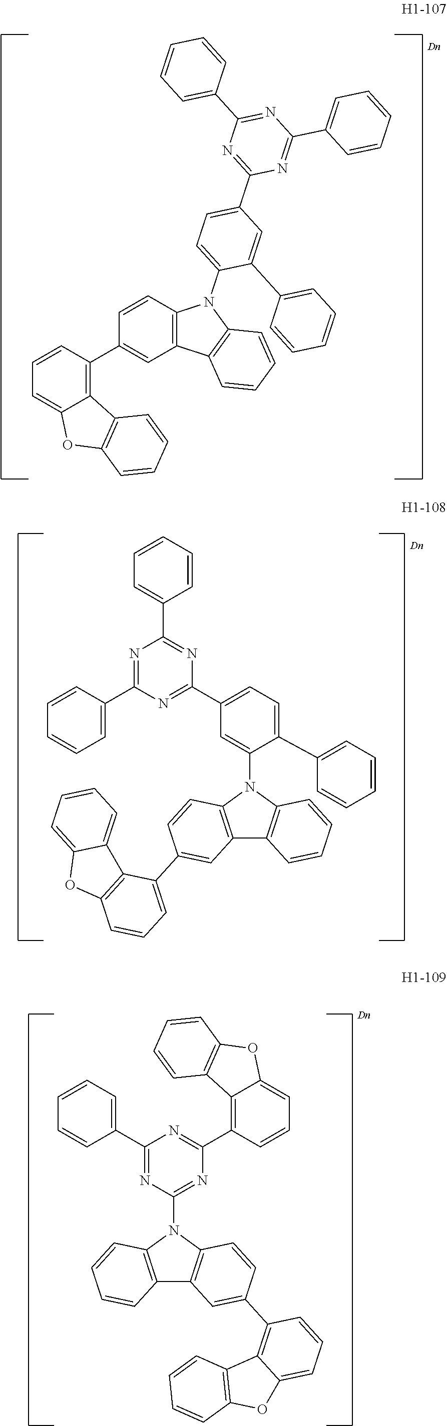 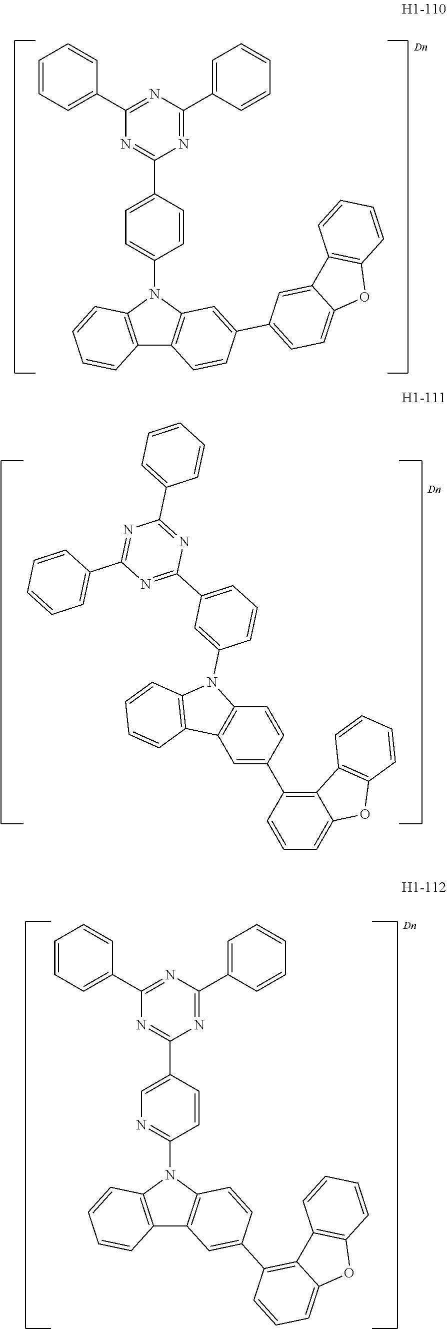 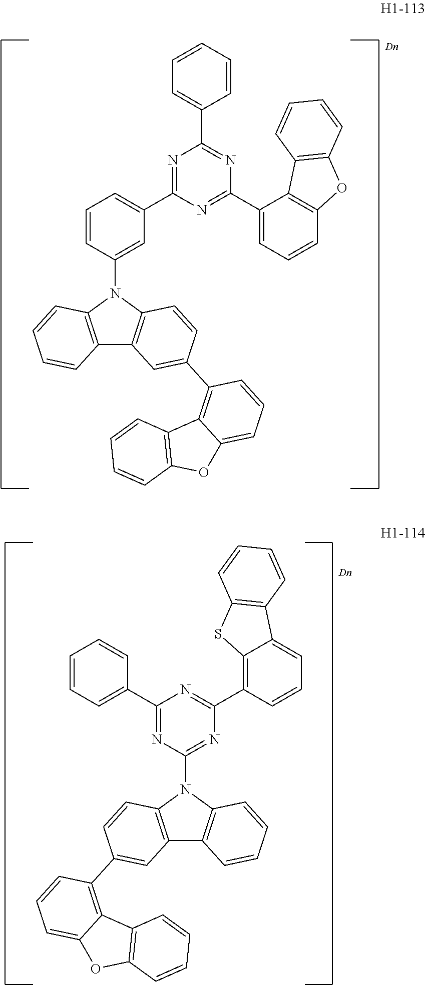 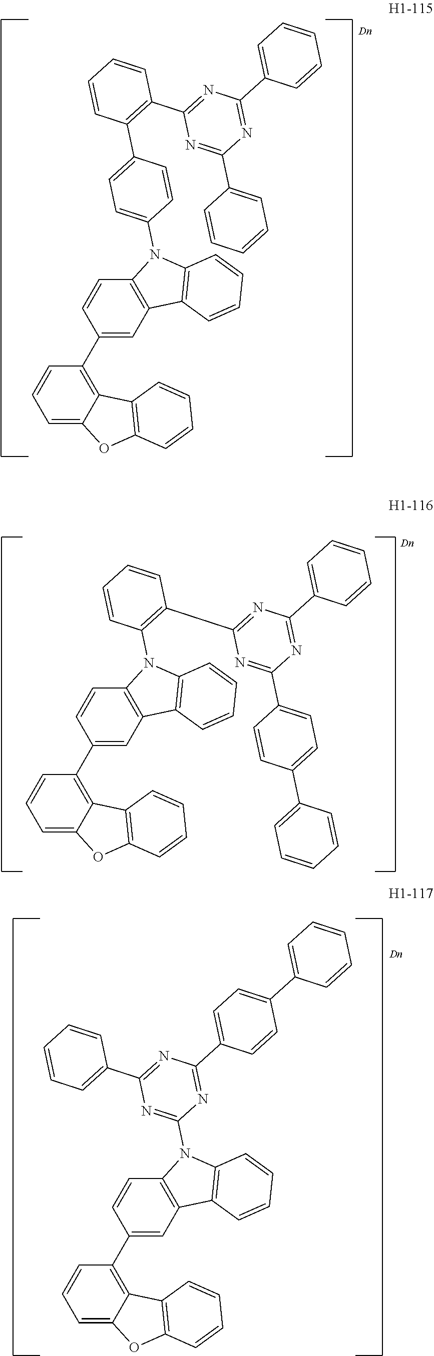 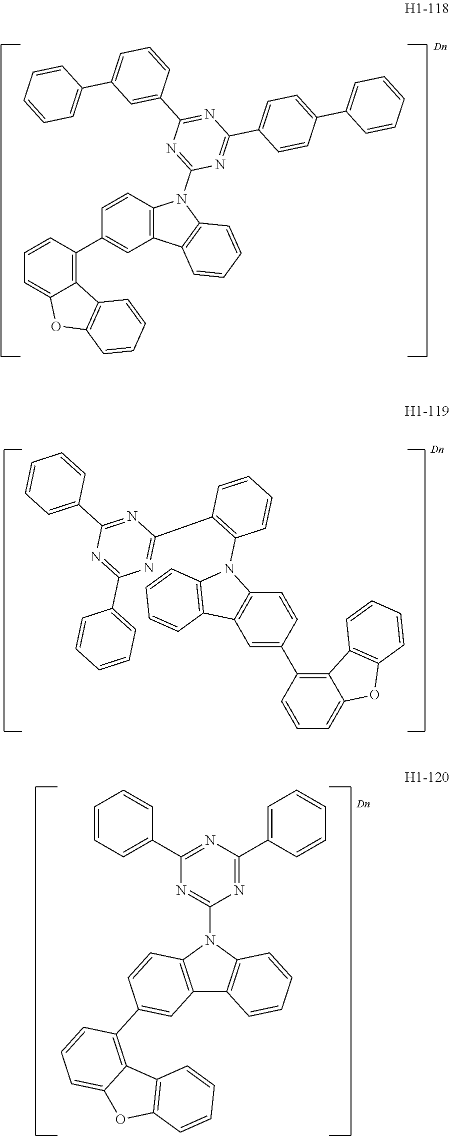 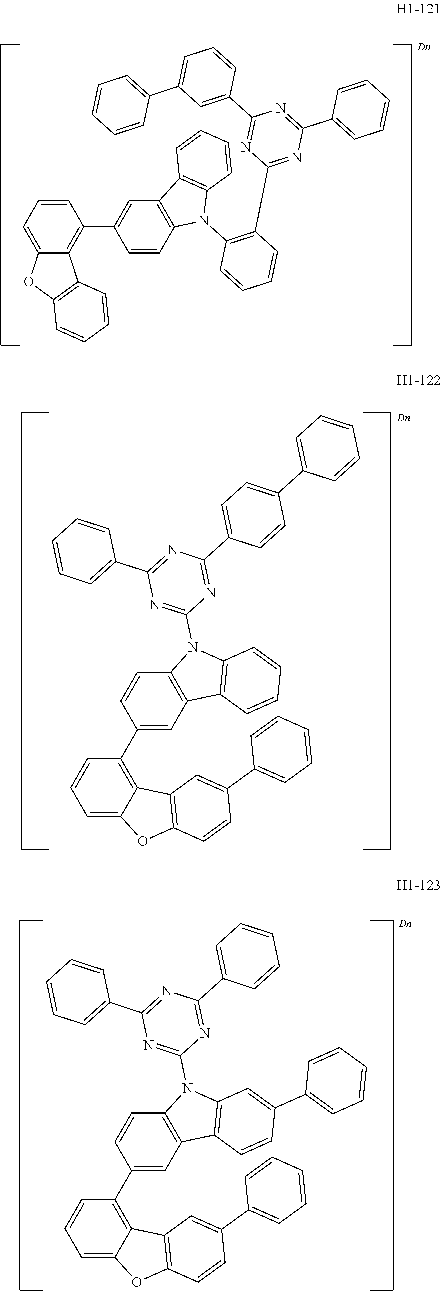 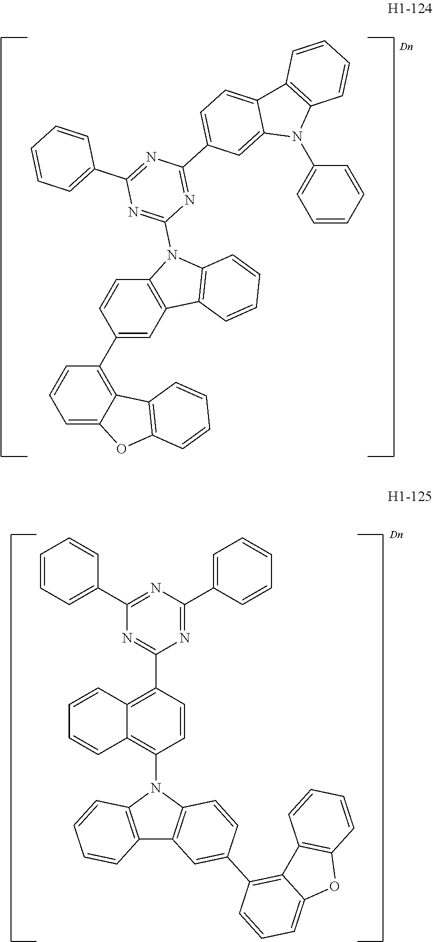 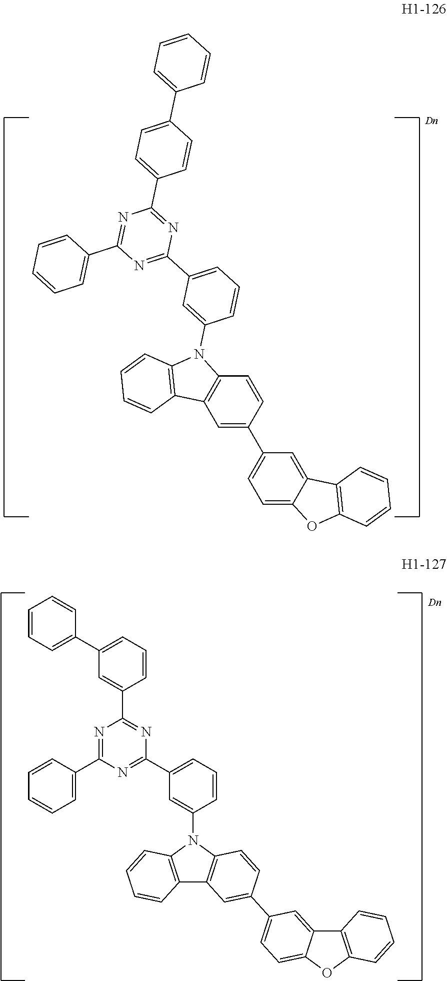 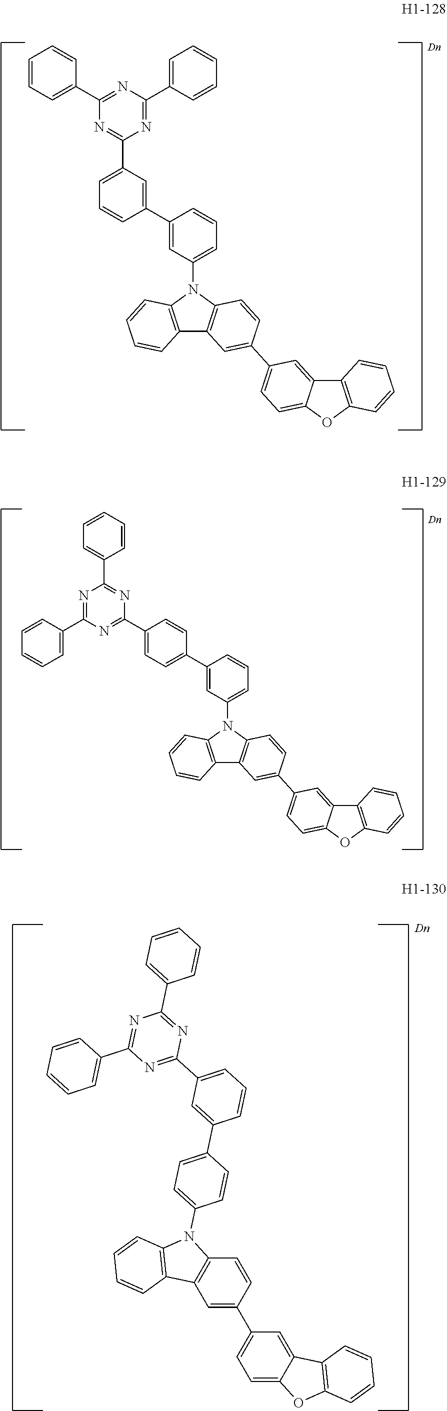 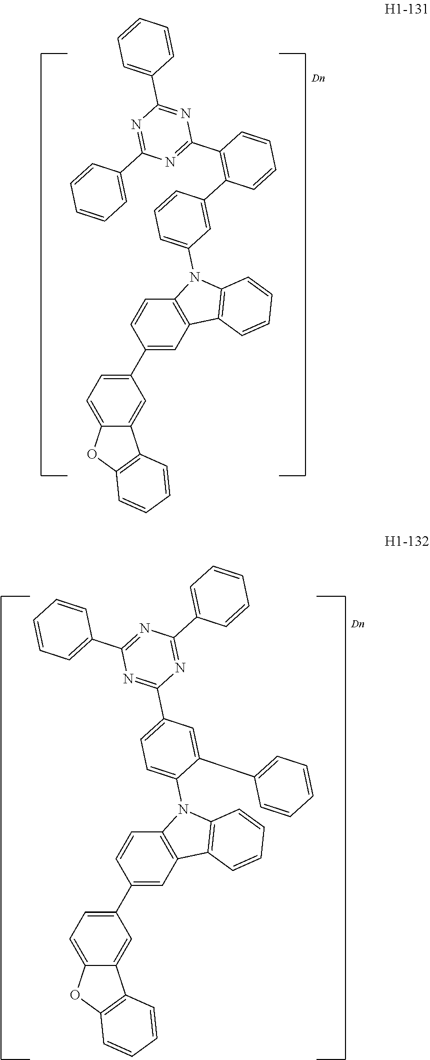 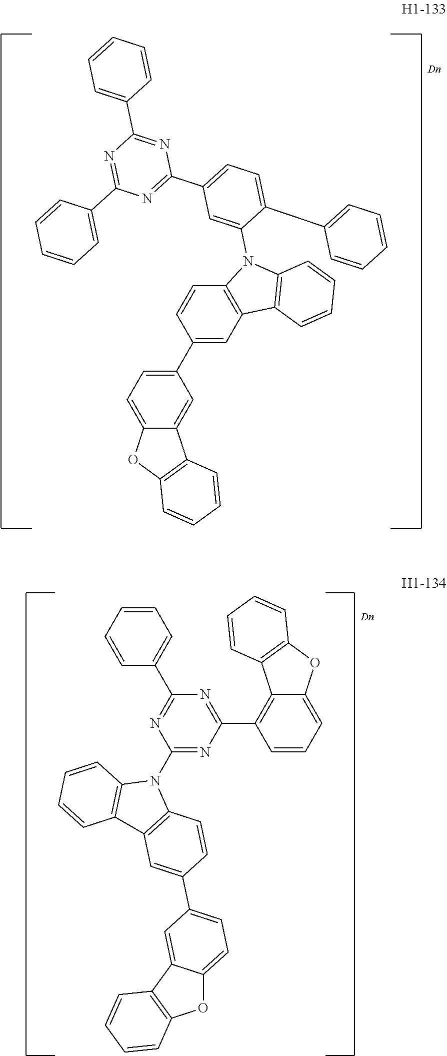 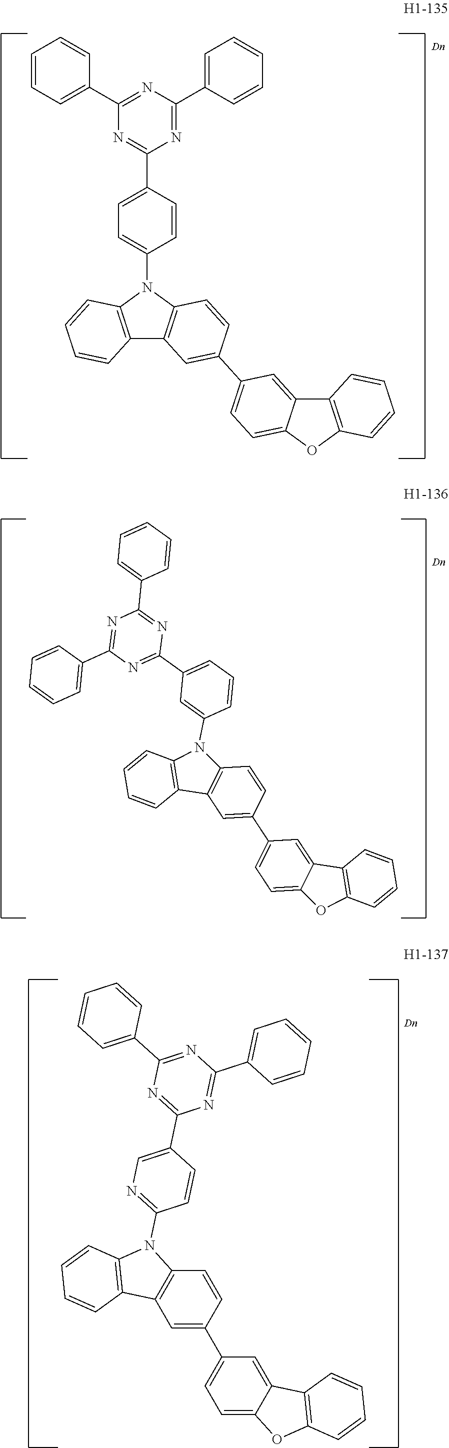 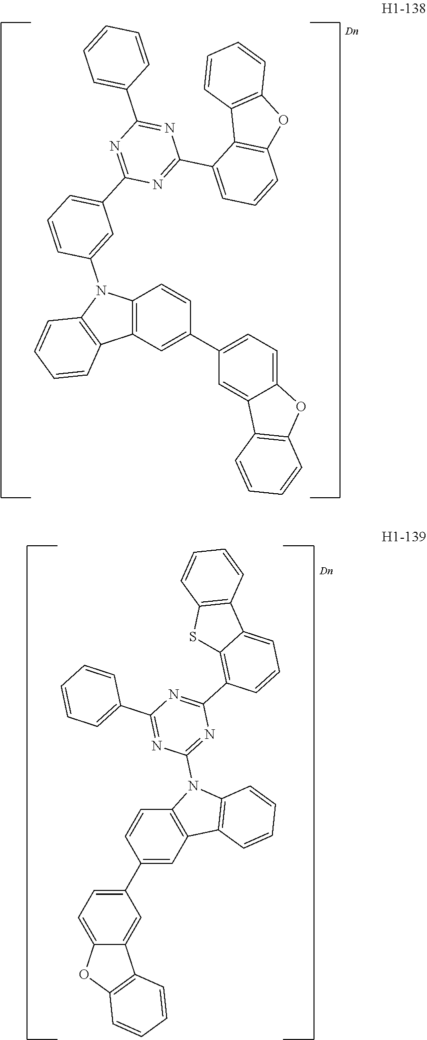 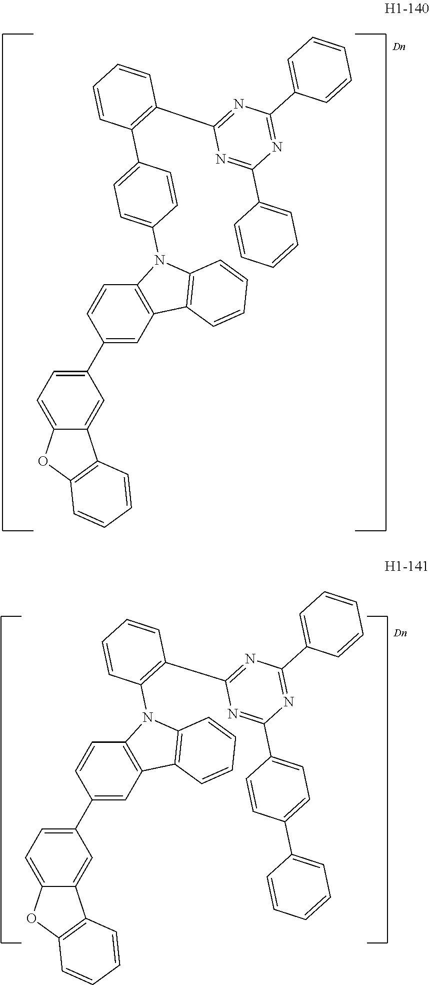 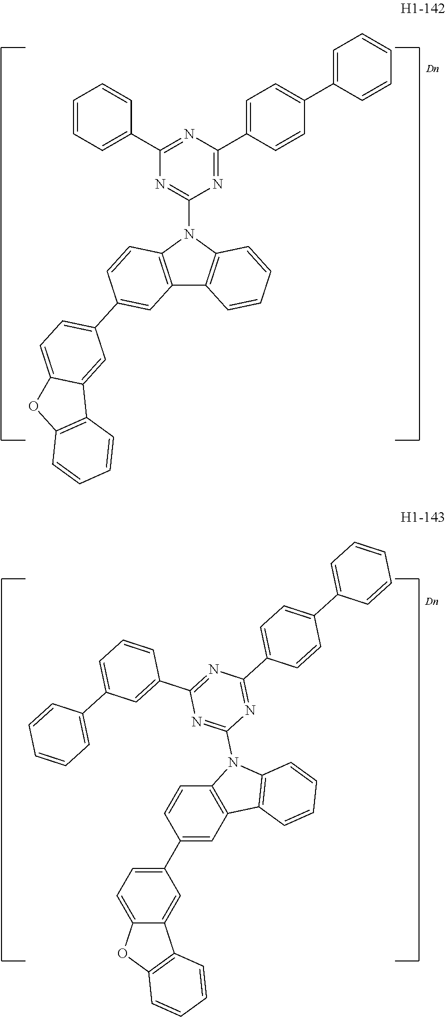 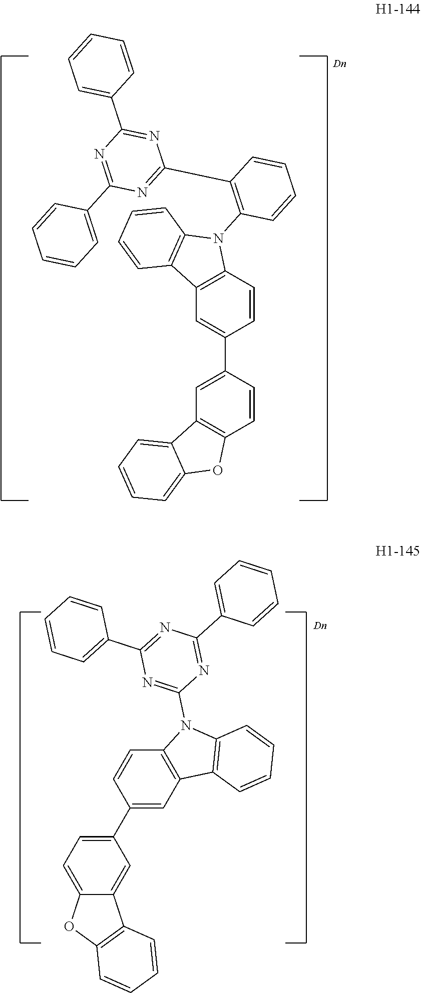 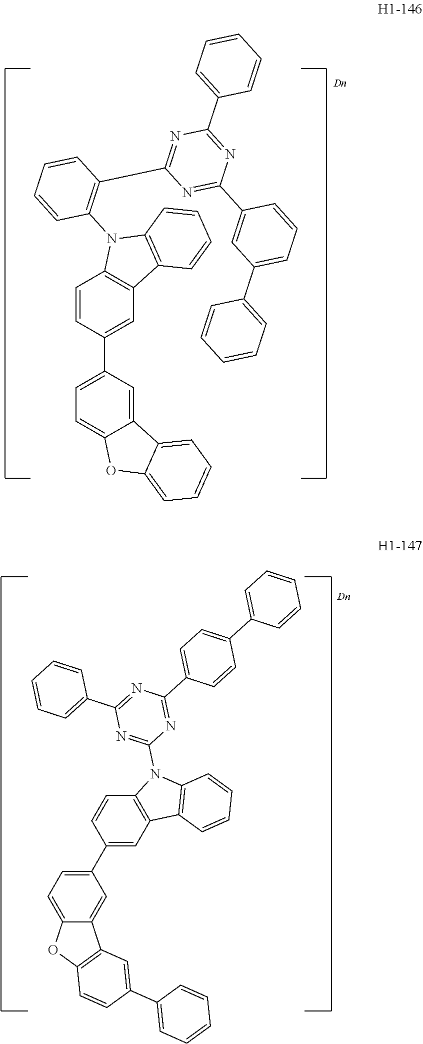 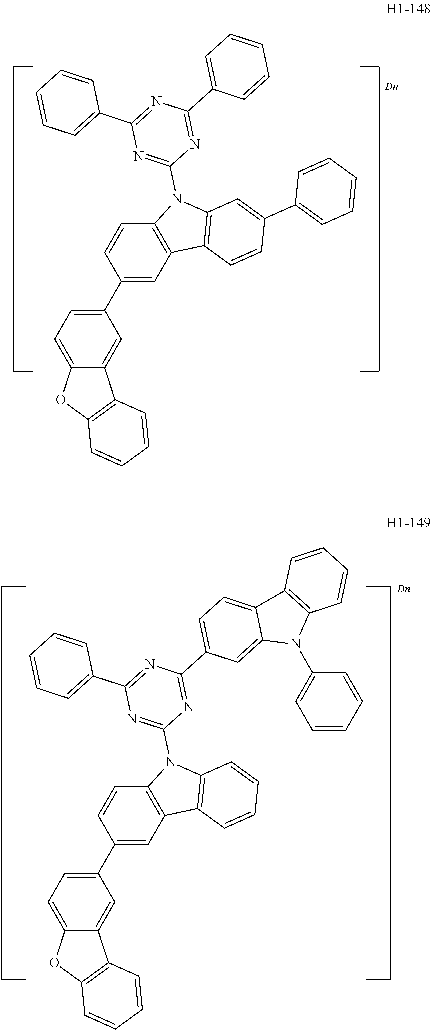 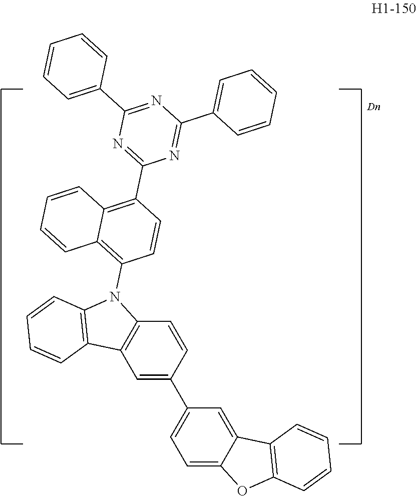 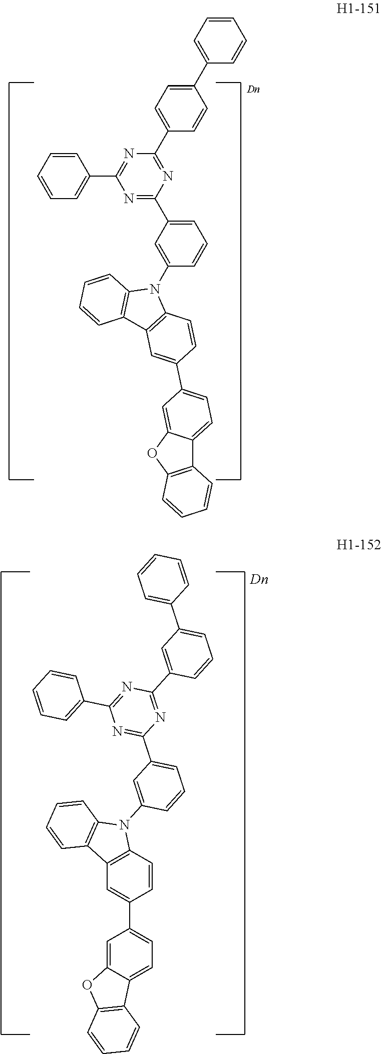 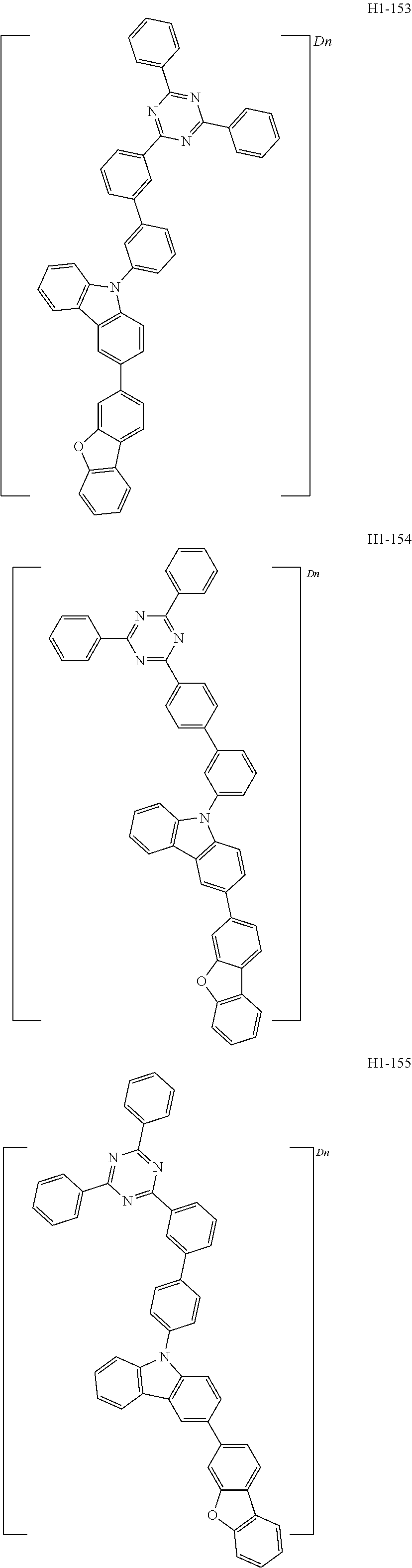 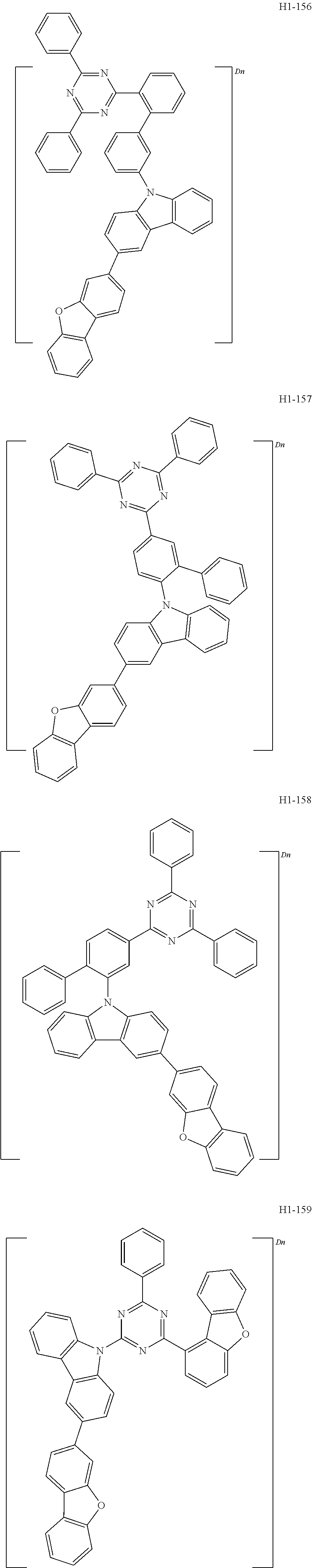 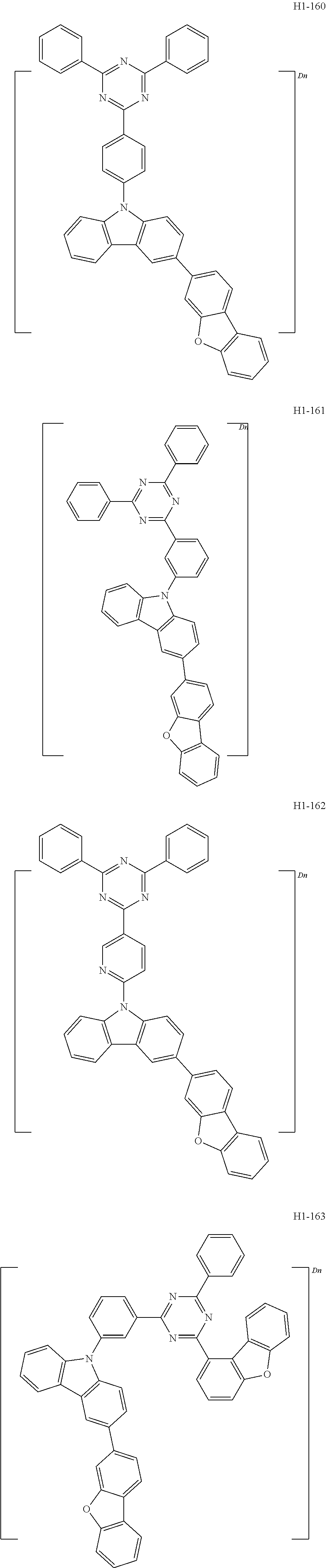 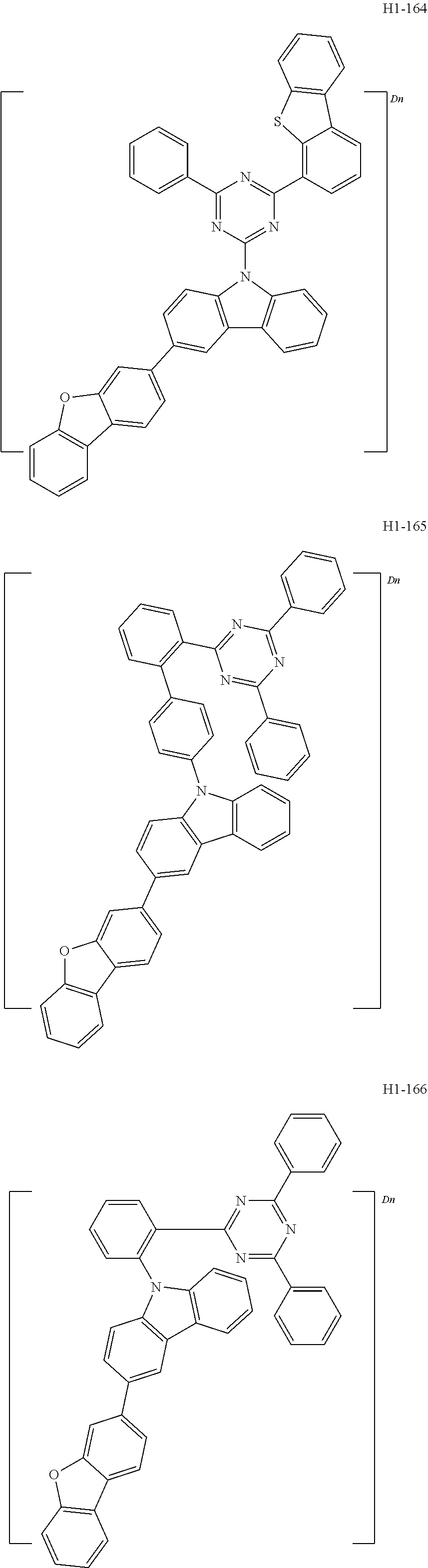 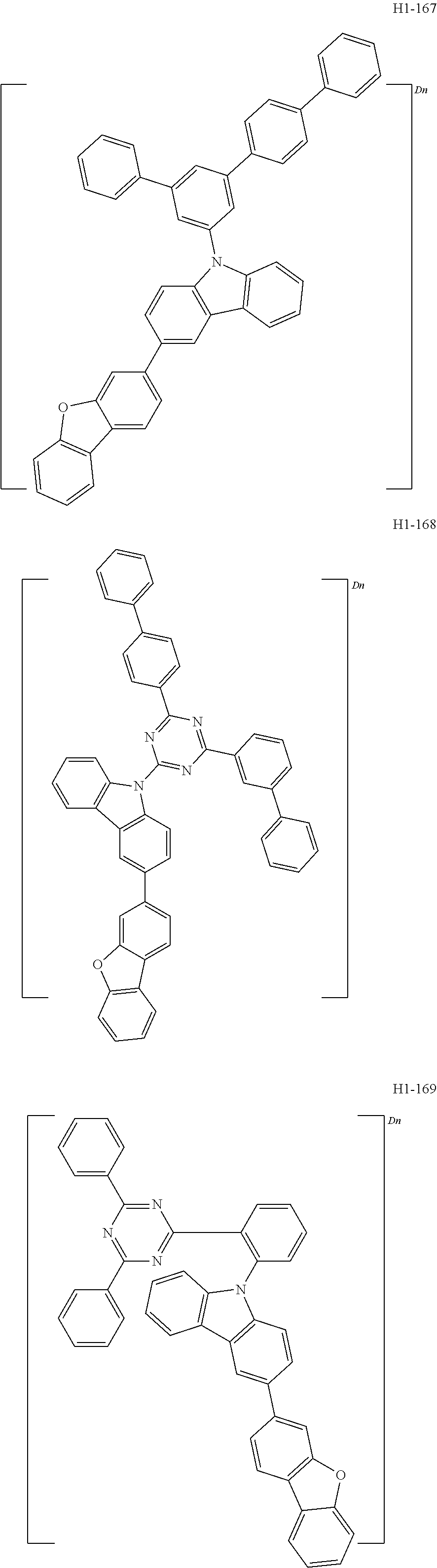 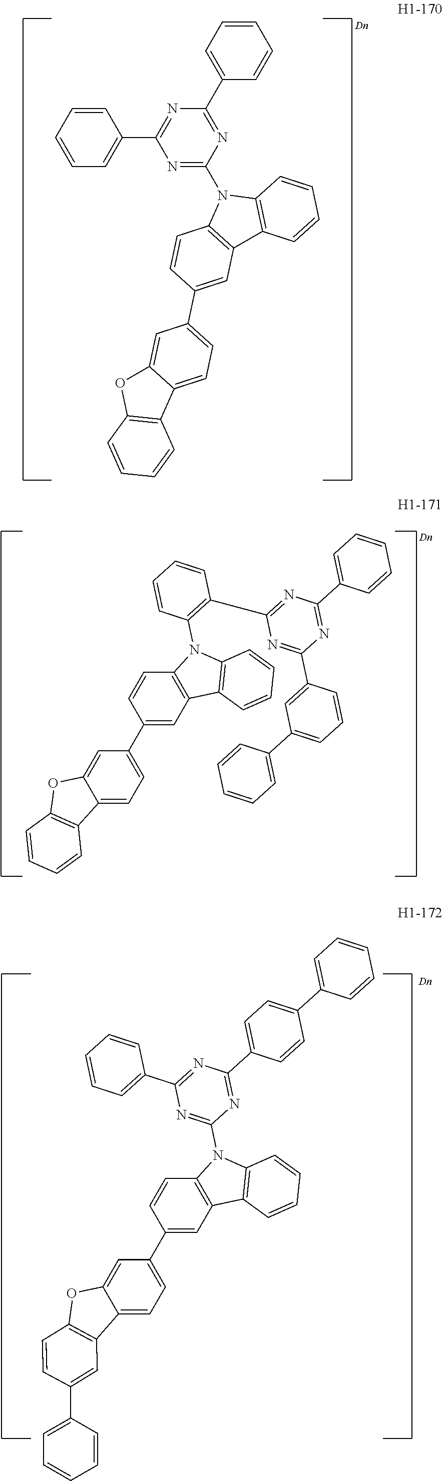 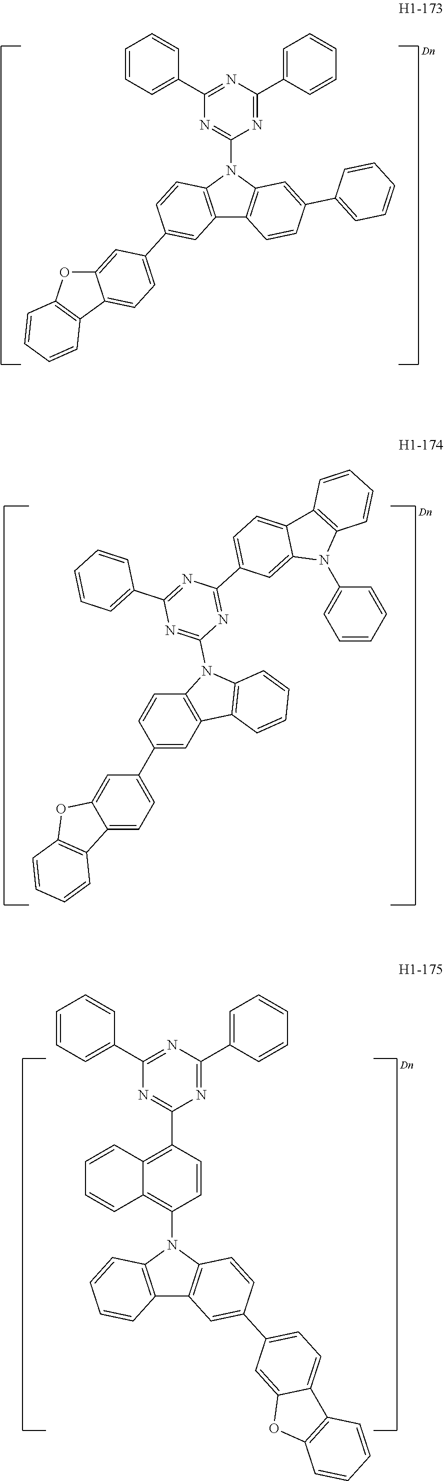 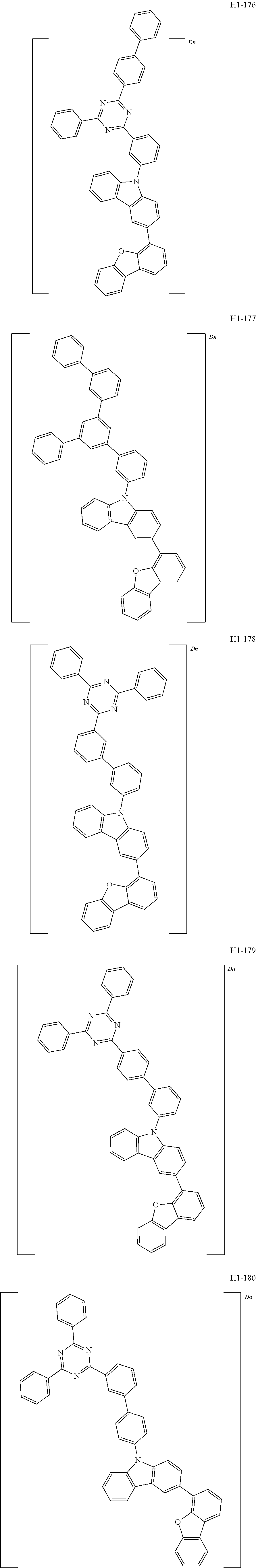 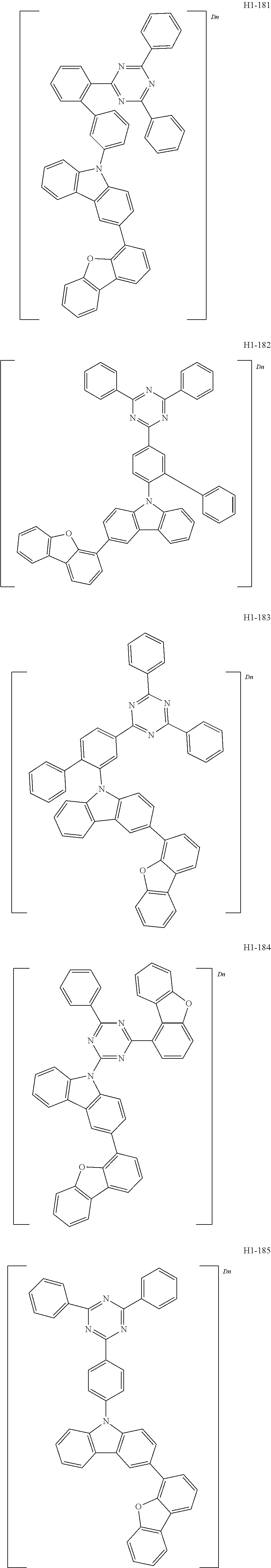 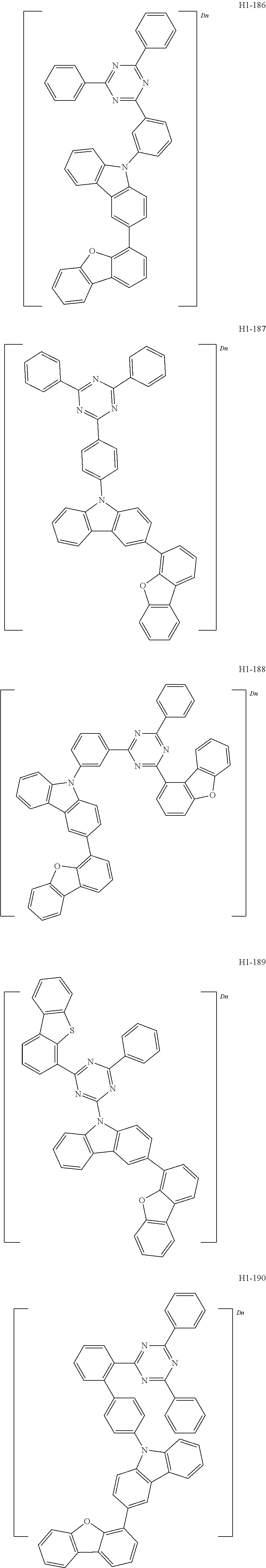 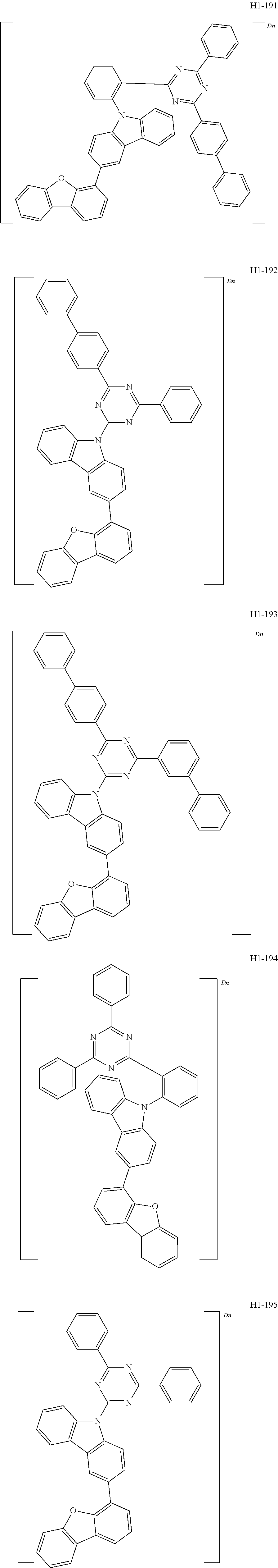 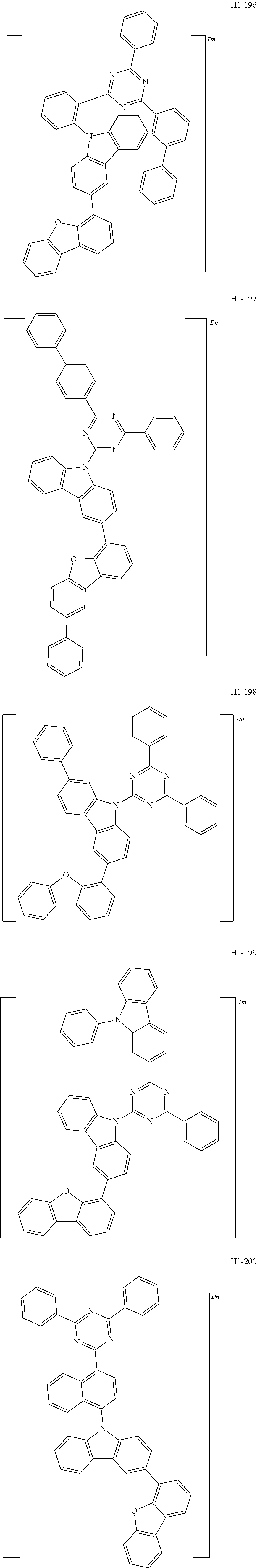 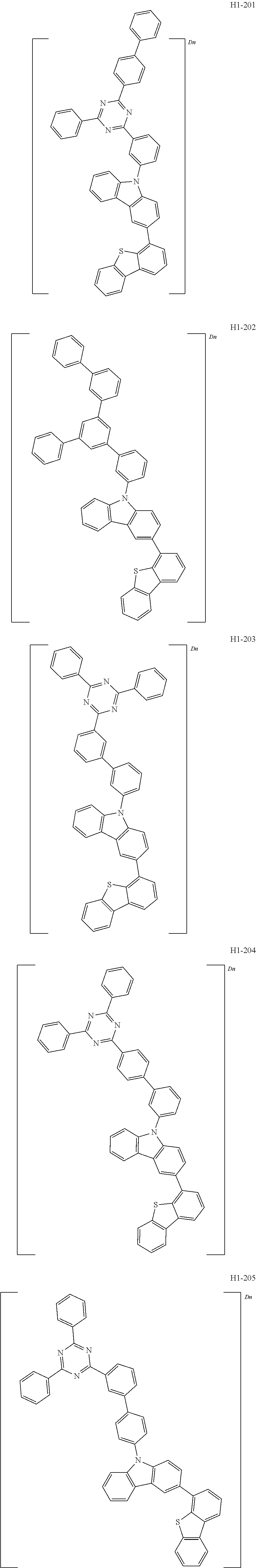 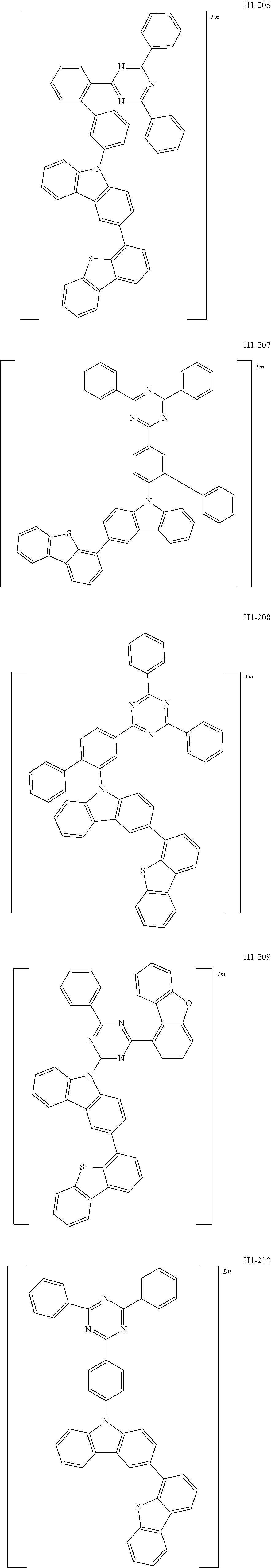 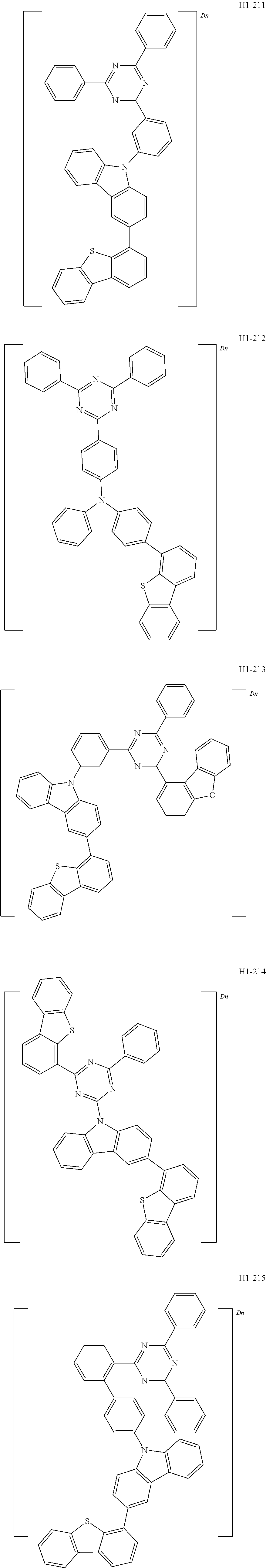 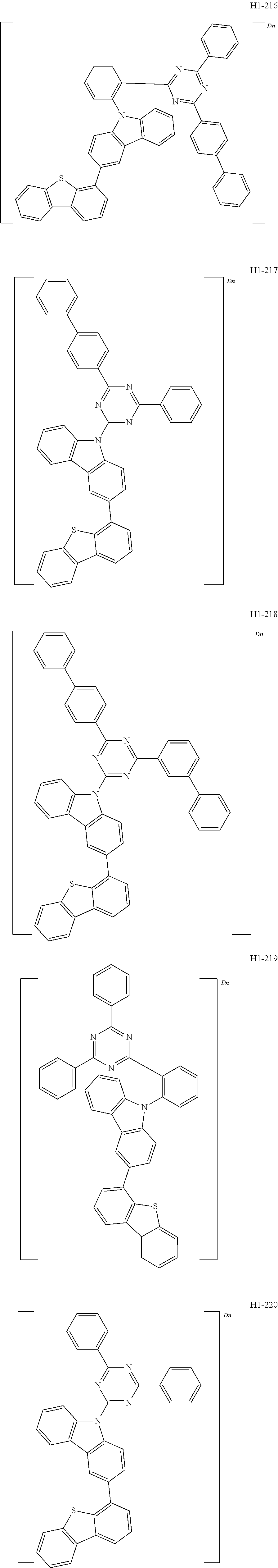 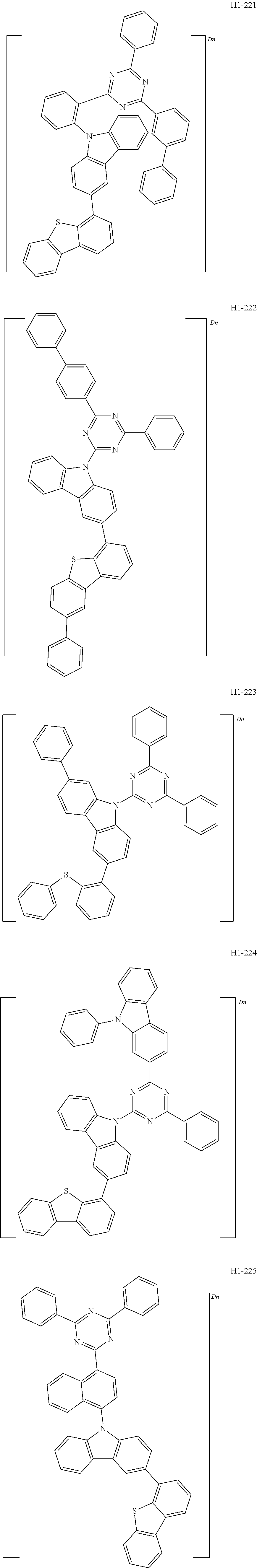 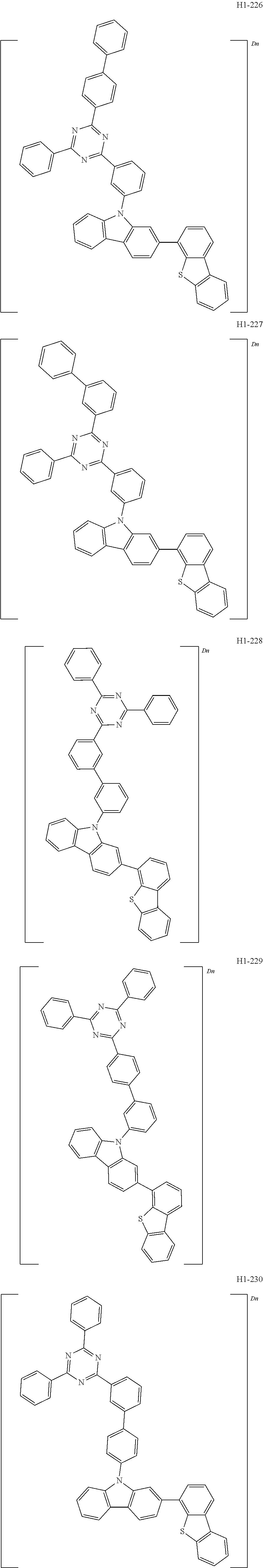 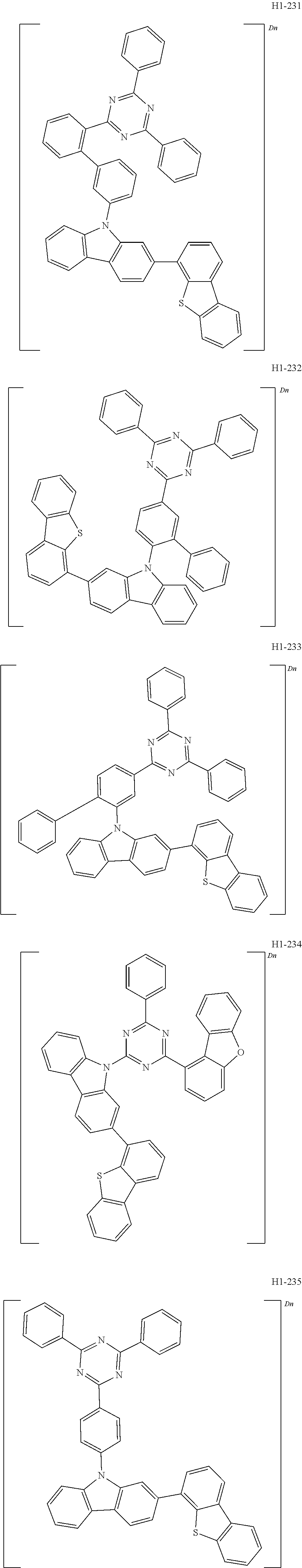 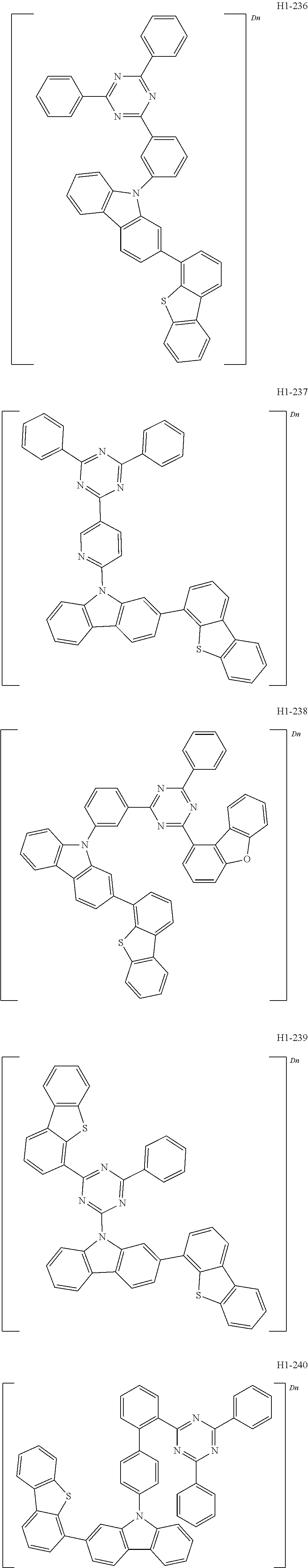 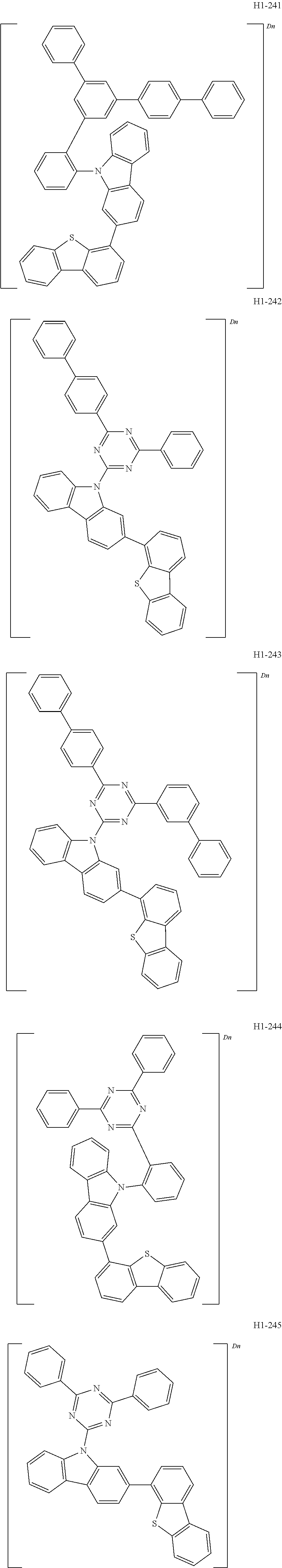 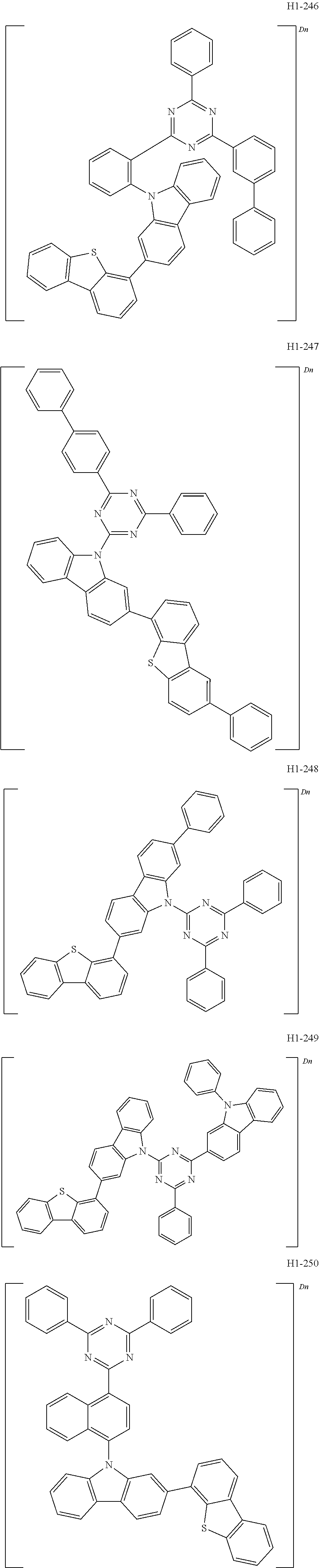 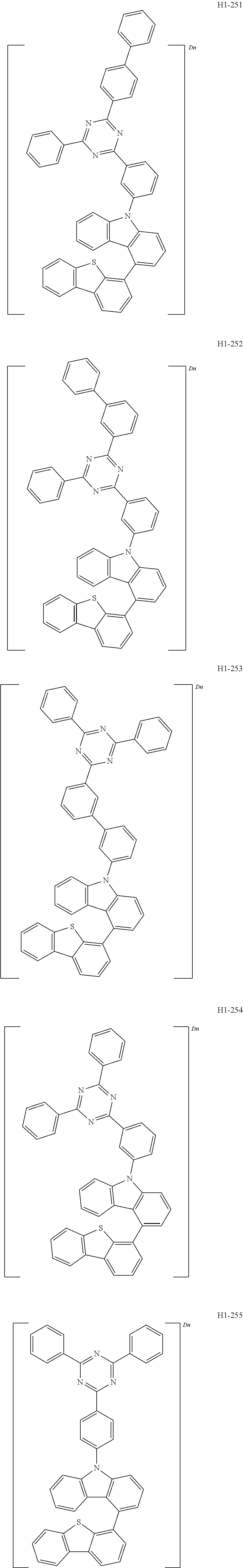 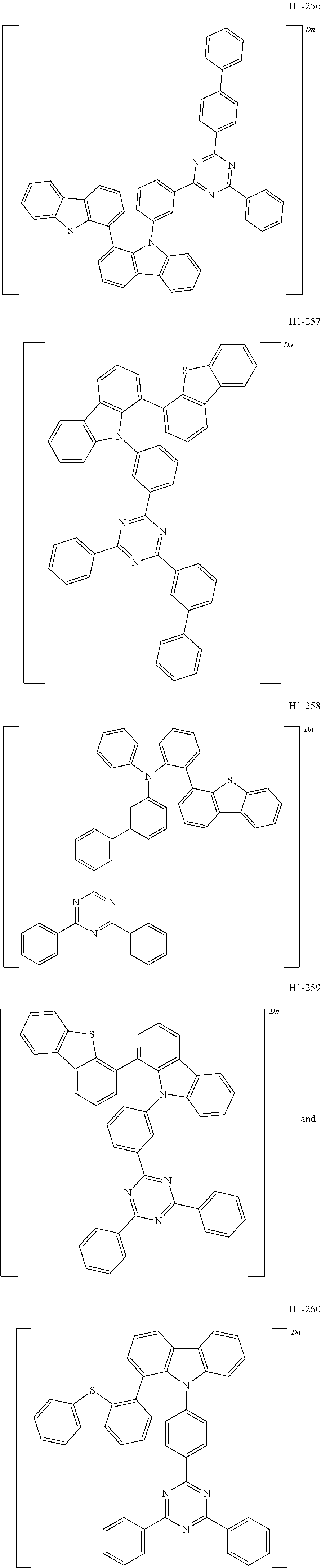
wherein, Dₙ represents that n number of hydrogens are replaced with deuterium; and n represents an integer of 5 to 50.

17. An organic electroluminescent device comprising the organic electroluminescent compound according to claim 13 or 15.
